(19) United States
(12) Patent Application Publication
(10) Pub. No.: US 2025/0179103 A1
(43) Pub. Date: Jun. 5, 2025

(54) 6-OXODECAHYDROPYRROLO[1,2-A][1,5]DIAZOCINE AND 6-OXODECAHYDRO-4H-PYRROLO[2,1-D][1,5]THIAZOCINE DERIVATIVES AS STAT3 AND STAT6 MODULATORS FOR THE TREATMENT OF CANCER AND INFLAMMATORY CONDITIONS

(71) Applicant: Recludix Pharma, Inc., San Diego, CA (US)

(72) Inventors: Neil Bifulco, Sudbury, MA (US); Howard Bregman, Melrose, MA (US); Giovanni Cianchetta, Boxford, MA (US); Brian Hodous, Arlington, MA (US); Samuel K. Reznik, Cambridge, MA (US); Yong Tang, West Roxbury, MA (US); Andrew Tasker, Simi Valley, CA (US); Rishi G. Vaswani, Lexington, MA (US); Ernest Allen Sickmier, Needham, MA (US); John Yeoman, Medford, MA (US); Xia Tian, Cambridge, MA (US)

(21) Appl. No.: 18/833,506

(22) PCT Filed: Feb. 27, 2023
(86) PCT No.: PCT/US2023/063318

§ 371 Date: Jul. 26, 2024

Related U.S. Application Data
(60) Provisional application No. 63/337,428, filed on May 2, 2022.
(60) Provisional application No. 63/313,916, filed on Feb. 25, 2022.

(51) Int. Cl. C07F 9/6561

(52) U.S. Cl. CPC C07F 9/6561

(57) ABSTRACT

Provided are compounds of Formula (I): and pharmaceutically acceptable salts and compositions thereof, which are useful for treating a variety of conditions associated with STAT3 and/or STAT6, such as e.g. cancer and inflammatory conditions. Preferred compounds are 6-oxodecahydropyrrolo[1,2-a][1,5]diazocine and 6-oxodecahydro-4H-pyrrolo[2,1-d][1,5]thiazocine derivatives.

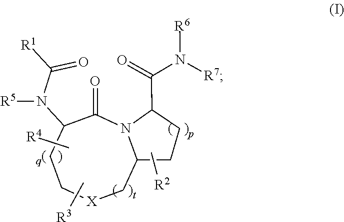

### RELATED APPLICATIONS

This application claims the benefit of priority to U.S. Provisional Application No. 63/313,916, filed Feb. 25, 2022 and U.S. Provisional Application No. 63/337,428, filed May 2, 2022, the entire contents of which are incorporated herein by reference.

### BACKGROUND

The Signal Transducer and Activator of Transcription (STAT) family of proteins consists of transcription factors that play an essential role in the regulation of cell processes, such as proliferation, differentiation, apoptosis and angiogenesis. Seven STAT genes have been identified in the human genome: STAT1, STAT2, STAT3, STAT4, STAT5a, STAT5b, and STAT6.

STAT3 has received particular attention because it is strongly associated with the promotion of tumor growth and immune evasion, and the only STAT family member whose genetic deletion results in embryonic lethality. Indeed, aberrantly elevated STAT3 activity has been estimated to occur in more than 70% of human cancers. Activated STAT3 mediates critical gene expression changes and molecular events that dysregulate cell growth and apoptosis, promote angiogenesis, invasion, metastasis, and the development of resistance to apoptosis, and suppress the host's immune surveillance of the tumor, thereby making constitutively-active STAT3 a critical mediator of carcinogenesis and tumor progression.

Another STAT protein that has gained recent interest is STAT6. Recent studies have shown that STAT6 signaling is essential for IL-4- and IL-13-induced epithelial mesenchymal transition (EMT) and aggressiveness of colorectal cancer cells (CRC) cells. STAT6 is involved in several aspects of inflammatory disease and other related conditions.

Given their role in the regulation of cell processes, modulating the activity of one or more STAT proteins, particularly STAT3 and/or STAT6, represent a pivotal area of investigation for the treatment of cancer, inflammatory conditions, and other therapeutic needs.

### SUMMARY

Provided herein are modulators of STAT3 and/or STAT6. Such modulators include those having the structural Formula I:

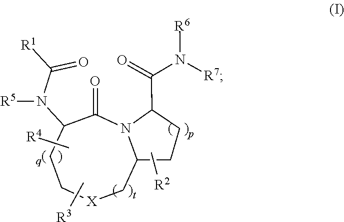

and pharmaceutically acceptable salts and compositions thereof, wherein R¹, R², R³, R⁴, R⁵, R⁶, R⁷, X, q, t and p are as described herein.

In one aspect, the disclosed compounds of Formula I and pharmaceutically acceptable salts thereof inhibit STAT3 and/or STAT6, and are useful in a variety of therapeutic applications such as, for example, in treating cancer and inflammatory conditions.

Pharmaceutical compositions comprising the compounds and pharmaceutically acceptable salts of the disclosed compounds of Formula I, as well as methods for their preparation are also included.

Methods of treating conditions responsive to the modulation of STAT3 and/or STAT6 using the disclosed compounds, pharmaceutically acceptable salts, and compositions thereof are also included.

### DETAILED DESCRIPTION

### 1. General Description of Compounds

In a second embodiment, provided herein is a compound of structural Formula I:

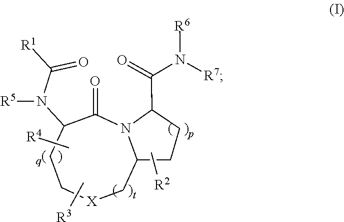

q is 0 or 1 and t is 0, 1, or 2, provided that at least one of q or t is 1;
p is 1 or 2;
X is selected from S, SO₂, —S(═O)═NH, and NR⁸;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}], wherein said aryl may be further optionally substituted with 1 or 2 groups independently selected from cyano, (C₁-C₄)alkoxy, and halo; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], —[P(O)[NH(AA)C(O)OR^{T}][NH(AA)C(O)OR^{T}], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 10-membered heteroaryl, —(C₁-C₄)[5- to 10-membered heteroaryl], phenyl, —(C₁-C₄)alkylphenyl, —C(O)RH, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 10-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, cyano, and 4- to 6-membered heterocyclyl optionally substituted with C₁-C₄alkyl;
R^{O} is selected from halo, (C₁-C₄)alkoxy, OH, phenyl, NH₂, —NH(C₁-C₁₀)alkyl, —N[(C₁-C₁₀)alkyl], (C₃₋₆cycloalkyl), 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano;
R^{Hb} is hydrogen or (C₁-C₄)alkyl;
AA is the residue of an alpha or beta natural or non-natural amino acid;
R^{T} and R^{Ty} are each independently selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} and R^{U} are each independently selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

### 2. Definitions

When used in connection to describe a chemical group that may have multiple points of attachment, a hyphen (-) designates the point of attachment of that group to the variable to which it is defined. For example, —NR^{c}C(O)R^{e} means that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

Unless otherwise specified, the term “alkyl” when used alone or as part of a larger moiety, such as “haloalkyl”, and the like, means saturated straight-chain or branched monovalent hydrocarbon radical.

The term “haloalkyl” includes mono, poly, and perhaloalkyl groups where the halogens are independently selected from fluorine, chlorine, bromine, and iodine.

“Alkoxy” means an alkyl radical attached through an oxygen linking atom, represented by —O-alkyl. For example, “(C₁-C₄)alkoxy” includes methoxy, ethoxy, propoxy, and butoxy.

“Haloalkoxy” is a haloalkyl group which is attached to another moiety via an oxygen atom such as, e.g., —OCHF₂ or —OCF₃.

The term “oxo” means the group ═O.

The term “imino” means the group ═NH.

Unless otherwise specified, the term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzoxadiazolyl, indazolyl, benzimidazolyl, benzthiazolyl, benzothiophenyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

Unless otherwise specified, the term “heterocyclyl” means a 4- to 12-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, tetrahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadiazolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, and the like. It will be understood that when specified, optional substituents on a heterocyclyl group may be present on any substitutable position and, include, e.g., the position at which the heterocyclyl is attached (where valency permits).

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “aryl” refers to an aromatic carbocyclic single ring or two fused ring system containing 6 to 10 carbon atoms. Examples include phenyl, indanyl, tetrahydronaphthalene, and naphthyl. In one aspect, the aryl is phenyl or naphthyl.

The terms “cycloalkyl”, used alone or as part of a larger moiety, refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The “residue of an amino acid” is the moiety remaining after formation of a bond between a reactive group in another compound (e.g., an amino group) and the carboxylic acid in the amino acid, after formation of a bond between a reactive group in another compound (e.g., a carboxylic acid) and the amino group in the amino acid, or both. As a consequence of the bond(s) formation, the carboxylic acid in the amino acid no longer has the OH group and instead has a bond between the carbonyl group and the reactive group in the compound; the amino group has only one hydrogen atom and instead has a bond between the reactive group in the other compound and the nitrogen of the amino group; or both. For example, the “residue of an alpha amino acid” can be depicted structurally as NH₂CR′R—C(O)—, —NHCR′R—C(O)OH or —NHCR′R—C(O)—; and the “residue of an beta amino acid” can be depicted structurally as or NH₂CR′RCH₂—C(O)—, —NHCR′RCH₂—C(O)OH or —NHCR′RCH₂—C(O)—, where R′ is H or C₁-C₆ alkyl and R is H or C₁-C₆ alkyl optionally substituted with 1 to 3 groups selected from halo, (C₁-C₃)alkoxy, OH, NH₂, —NH(C₁-C₄ alkyl), —N[(C₁-C₄ alkyl)]₂, SH, S(C₁-C₄ alkyl), imino, COOH, —COO(C₁-C₄ alkyl), —CO(C₁-C₄ alkyl), —CONH(C₁-C₄ alkyl)phenyl, phenyl, and 5- to 10-membered heteroaryl, wherein said C₁-C₆ alkyl may also be optionally interrupted by a sulfur or nitrogen heteroatom and wherein said phenyl is optionally substituted with 1 to 3 groups selected from OH, cyano, (C₁-C₄ alkyl), and halo(C₁-C₄ alkyl); or R is taken together with the nitrogen atoms from the alpha or beta amino acid residue to form a 4- to 6-membered heterocyclyl. For naturally occurring alpha amino acid (i.e., amino acids that occur in nature), R′ is H and R is selected from hydrogen, methyl, isopropyl, —CH₂CH(CH₃)₂, —(CH₂)₂SCH₃, —CH(CH₃)(CH₂CH₃), CH₂OH, —CH(OH)(CH₃), CH₂SH, —CH₂C(O)NH₂, —(CH₂)₂C(O)NH₂, benzyl, p-hydroxybenzyl, —CH₂(indolyl), —(CH₂)₄NH₂, —(CH₂)₃NHC(═NH₂)NH₂, —CH₂(imidazolyl), —(CH₂)COOH, and —(CH₂)₂COOH; or R taken together with the nitrogen atom of the alpha or beta amino acid residue forms a pyrrolidinyl ring.

Non-natural amino acids are known in the art and include e.g., alpha-alkyl amino acids (e.g., alpha methyl), alpha-alkylalkoxy amino acids (e.g., alpha —CH₂OCH₃), N-methyl amino acids, homo-amino acids, etc.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

In certain instances, compounds were isolated and tested as a 1:1 mixture of diastereomers. In such cases, the relative stereochemistry is denoted by the term “rel-” in the compound name and by the use of flat bonds instead of wedges. For example, ((2-(((5S,8S,10aR)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic acid, having the structure:

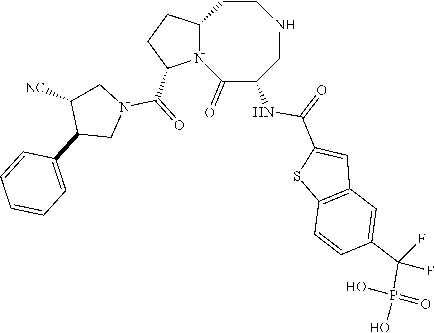 [SMILES: N#C[C@@H]1CN(C(=O)[C@@H]2CC[C@@H]3CCNC[C@H](NC(=O)c4cc5cc(C(F)(F)P(=O)(O)O)ccc5s4)C(=O)N32)C[C@H]1c1ccccc1]

means that the substituents about the pyrrolidine ring are trans and encompass a mixture of both diastereomers

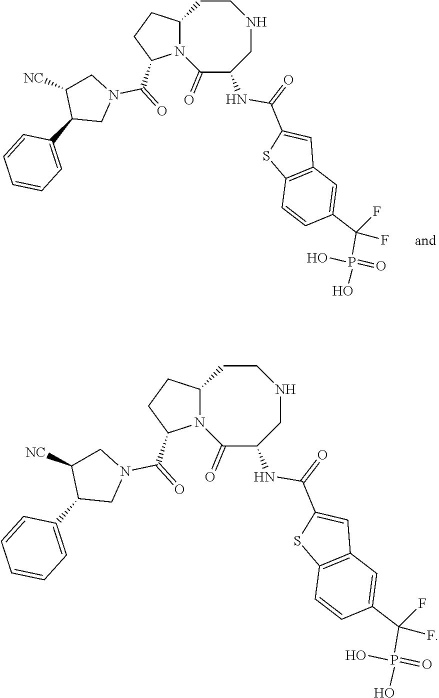 [SMILES: N#C[C@@H]1CN(C(=O)[C@@H]2CC[C@@H]3CCNC[C@H](NC(=O)c4cc5cc(C(F)(F)P(=O)(O)O)ccc5s4)C(=O)N32)C[C@H]1c1ccccc1 ; N#C[C@H]1CN(C(=O)[C@@H]2CC[C@@H]3CCNC[C@H](NC(=O)c4cc5cc(C(F)(F)P(=O)(O)O)ccc5s4)C(=O)N32)C[C@@H]1c1ccccc1]

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that is sufficient to achieve the desired therapeutic effect (such as treatment of a condition recited herein) under the conditions of administration e.g., a dosage of between 0.01-100 mg/kg body weight/day.

### 3. Compounds

In a first embodiment, provided is a compound of structural Formula I:

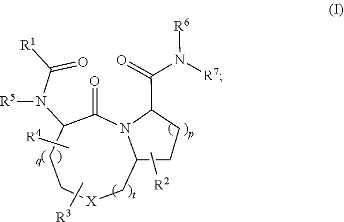

or a pharmaceutically acceptable salt thereof, wherein the variables are as described above.

In a second embodiment, R¹ in the compound of Formula I, or a pharmaceutically acceptable salt thereof, is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 6-membered heteroaryl, —(C₁-C₄)[5- to 6-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 6-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R⁰ and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano; and
R^{O} is selected from halo, (C₁-C₄)alkoxy, OH, phenyl, NH₂, —NH(C₁-C₁₀)alkyl, —N[(C₁-C₁₀)alkyl], 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano, wherein the remaining variable are as described above for Formula I.

In a third embodiment, the compound of Formula I is of the structural Formula II:

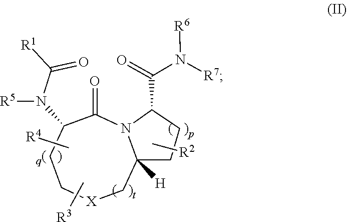

or a pharmaceutically acceptable salt thereof, wherein the variables are as described above for Formula I or the second embodiment.

In a fourth embodiment, p in the compound of Formula I or II, or a pharmaceutically acceptable salt thereof, is 1, wherein the remaining variables are as described above for Formula I or the second embodiment.

In a fifth embodiment, R² in the compound of Formula I or II, or a pharmaceutically acceptable salt thereof, is hydrogen, wherein the remaining variables are as described above for Formula I or any one of the second or fourth embodiments.

In a sixth embodiment, R³ in the compound of Formula I or II, or a pharmaceutically acceptable salt thereof, is hydrogen, wherein the remaining variables are as described above for Formula I or any one of the second, fourth or fifth embodiments.

In a seventh embodiment, R⁴ in the compound of Formula I or II, or a pharmaceutically acceptable salt thereof, is hydrogen, wherein the remaining variables are as described above for Formula I or any one of the second and fourth to sixth embodiments.

In an eighth embodiment, q in the compound of Formula I or II, or a pharmaceutically acceptable salt thereof, is 0 and t is 2, wherein the remaining variables are as described above for Formula I or any one of the second and fourth to seventh embodiments. Alternatively, as part of an eighth embodiment, q in the compound of Formula I or II, or a pharmaceutically acceptable salt thereof, is 1 and t is 1, wherein the remaining variables are as described above for Formula I or any one of the second and fourth to seventh embodiments. In another alternative, as part of an eighth embodiment, q in the compound of Formula I or II, or a pharmaceutically acceptable salt thereof, is 0 and t is 1, wherein the remaining variables are as described above for Formula I or any one of the second and fourth to seventh embodiments.

In a ninth embodiment, the compound of Formula I is of the structural Formula III, IV, V, VI, VII, or VIII:

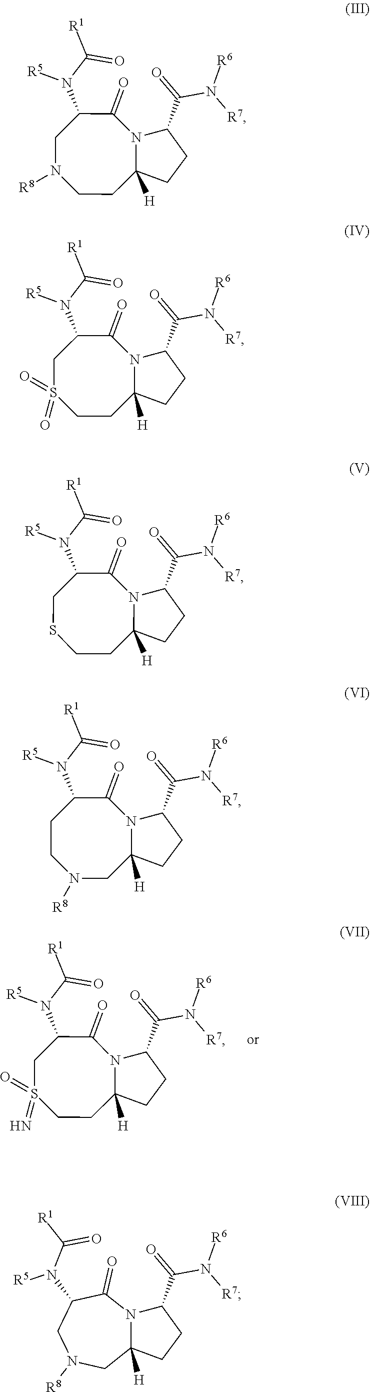

or a pharmaceutically acceptable salt thereof, wherein the variables are as described above for Formula I or the second embodiment.

In a tenth embodiment, R⁵ in the compound of structural Formula I, II, III, IV, V, VI, VII, or VIII, or a pharmaceutically acceptable salt thereof, is hydrogen, wherein the remaining variables are as described above for Formula I or any one of the second and fourth to eighth embodiments.

In an eleventh embodiment, R¹ in the compound of structural Formula I, II, III, IV, V, VI, VII, or VIII, or a pharmaceutically acceptable salt thereof, is selected from 8- to 10-membered fused bicyclic heteroaryl and aryl, each of which are substituted with —CR^{1a}R^{2a}P(O)OR^{1b}OR^{2b}, wherein the remaining variables are as described above for Formula I or any one of the second, fourth to eighth, and tenth embodiments. Alternatively, as part of an eleventh embodiment, R¹ in the compound of structural Formula I, II, III, IV, V, VI, VII, or VIII, or a pharmaceutically acceptable salt thereof, is selected from benzothiophenyl, indolyl, naphthalenyl, thienopyridinyl, benzothiazoyl, quinolinyl, isoquinolinyl, 4,5,6,7-tetrahydropyrrolopyridinyl, 5,6,7,8-tetrahydroimidazopyrazinyl, each of which are substituted with —CR^{1a}R^{2a}P(O)OR^{1b}OR^{2b}, wherein the remaining variables are as described above for Formula I or any one of the second, fourth to eighth, and tenth embodiments. In another alternative, as part of an eleventh embodiment, R¹ in the compound of structural Formula I, II, III, IV, V, VI, VII, or VIII, or a pharmaceutically acceptable salt thereof, is selected from

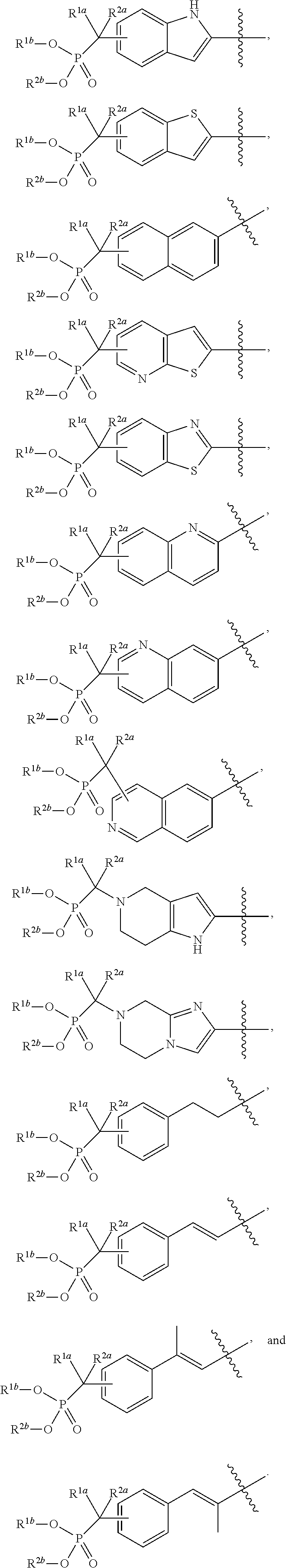

wherein the remaining variables are as described above for Formula I or any one of the second, fourth to eighth, and tenth embodiments. In another alternative, as part of an eleventh embodiment, R¹ in the compound of structural Formula I, II, III, IV, V, VI, VII, or VIII, or a pharmaceutically acceptable salt thereof, is

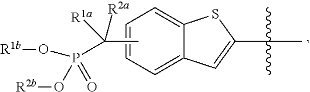

wherein the remaining variables are as described above for Formula I or any one of the second, fourth to eighth, and tenth embodiments.

In a twelfth embodiment, R^{1a} in the compound of structural Formula I, II, III, IV, V, VI, VII, or VIII, or a pharmaceutically acceptable salt thereof, is hydrogen and R^{2a} is fluoro or R^{1a} is fluoro and R^{2a} is fluoro, wherein the remaining variables are as described above for Formula I or any one of the second, fourth to eighth, tenth, and eleventh embodiments. Alternatively, as part of an twelfth embodiment, R^{1a} in the compound of structural Formula I, II, III, IV, V, VI, VII, or VIII, or a pharmaceutically acceptable salt thereof, is fluoro and R^{2a} is fluoro, wherein the remaining variables are as described above for Formula I or any one of the second, fourth to eighth, tenth, and eleventh embodiments.

In a thirteenth embodiment, R^{1b} and R^{2b} in the compound of structural Formula I, II, III, IV, V, VI, VII, or VIII, or a pharmaceutically acceptable salt thereof, are each independently selected from hydrogen, (C₁-C₄)alkyl, —[(C₁-C₄)alkyl]-OC(O)—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-OC(O)NR^{2c}R^{2d}], [(C₁-C₄)alkyl]-OC(O)O-[5- to 7-membered heterocyclyl], and phenyl, wherein the 5- to 7-membered heterocyclyl of [(C₁-C₄)alkyl]-OC(O)O-[5- to 7-membered heterocyclyl] is optionally substituted with C(O)OR^{2c}, wherein any of the (C₁-C₄)alkyl groups are optionally substituted with 1 or 2 (C₁-C₄)alkyl, wherein the remaining variables are as described above for Formula I or any one of the second, fourth to eighth, and tenth to twelfth embodiments. Alternatively, as part of a thirteenth embodiment, R^{1b} and R^{2b} in the compound of structural Formula I, II, III, IV, V, VI, VII, or VIII, or a pharmaceutically acceptable salt thereof, are each —[(C₁-C₄)alkyl]-OC(O)—[(C₁-C₄)alkyl], wherein the remaining variables are as described above for Formula I or any one of the second, fourth to eighth, and tenth to twelfth embodiments. In another alternative, as part as part of a thirteenth embodiment, R^{1b} and R^{2b} in the compound of structural Formula I, II, III, IV, V, VI, VII, or VIII, or a pharmaceutically acceptable salt thereof, are each hydrogen, wherein the remaining variables are as described above for Formula I or any one of the second, fourth to eighth, and tenth to twelfth embodiments.

In a fourteenth embodiment, —CR^{1a}R^{2a}P(O)OR^{1b}OR^{2b} in the compound of structural Formula I, II, III, IV, V, VI, VII, or VIII, or a pharmaceutically acceptable salt thereof, is selected from

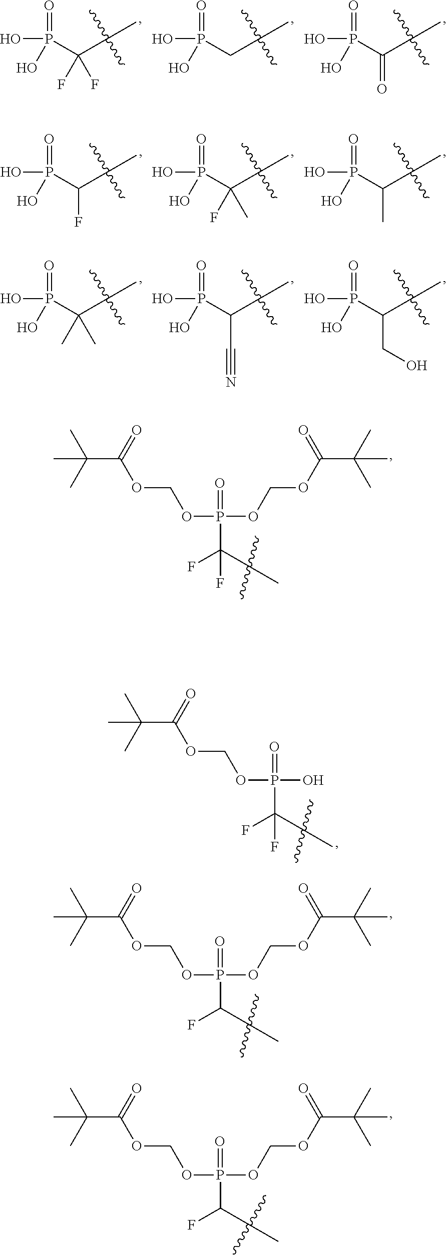

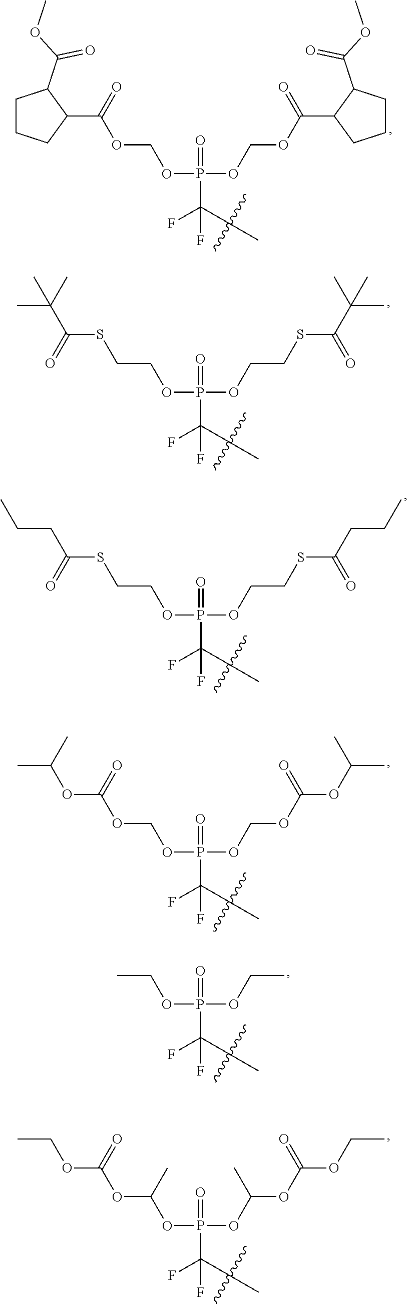

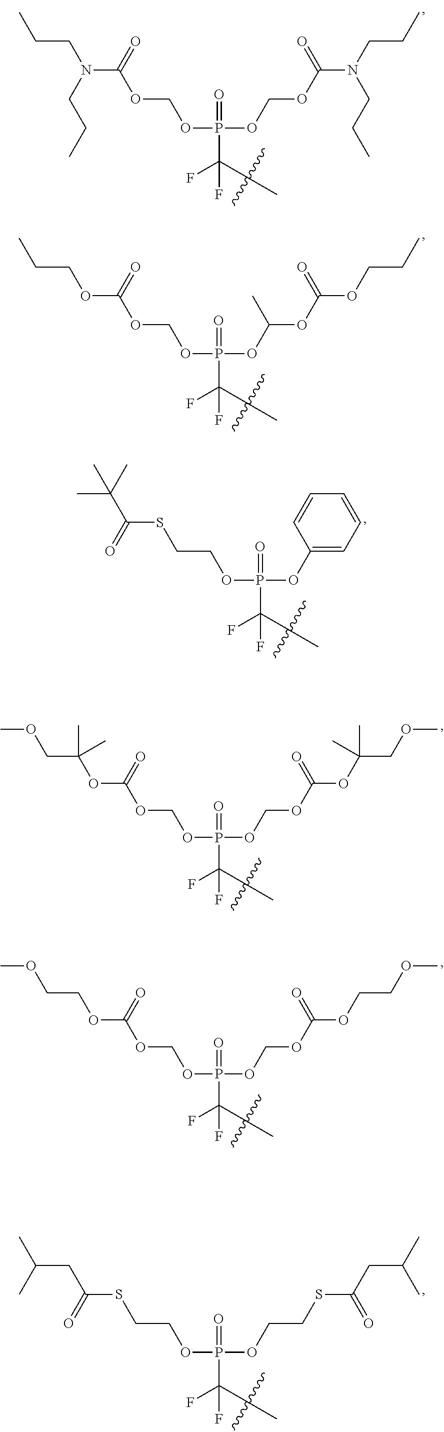

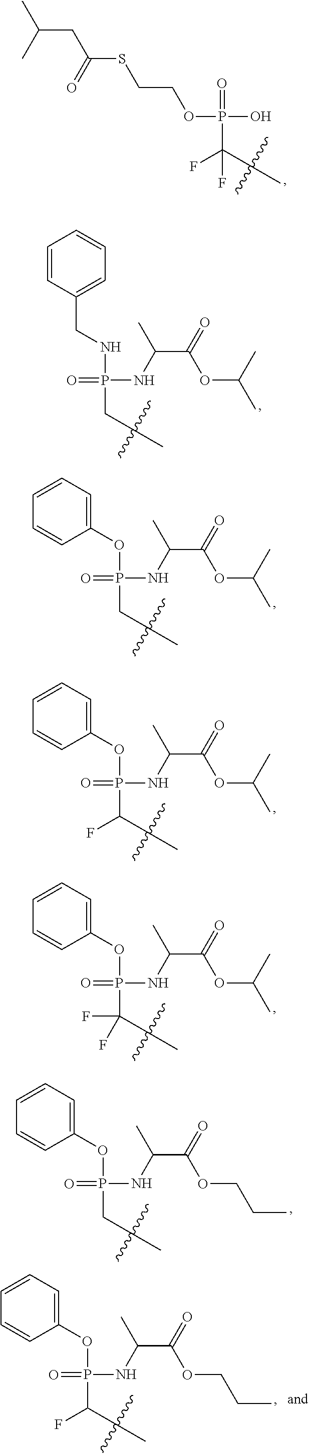

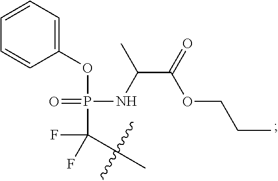 [SMILES: *C(F)(F)P(=O)(NC(C)C(=O)OCCC)Oc1ccccc1]

wherein the remaining variables are as described above for Formula I or any one of the second, fourth to eighth, and tenth to thirteenth embodiments. Alternatively, as part of a fourteenth embodiment, —CR^{1a}R^{2a}P(O)OR^{1b}OR^{2b} in the compound of structural Formula I, II, III, IV, V, VI, VII, or VIII, or a pharmaceutically acceptable salt thereof, is selected from

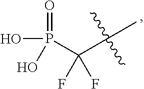 [SMILES: *C(F)(F)P(=O)(O)O]

wherein the remaining variables are as described above for Formula I or any one of the second, fourth to eighth, and tenth to thirteenth embodiments.

In a fifteenth embodiment, R⁷ in the compound of structural Formula I, II, III, IV, V, VI, VII, or VIII, or a pharmaceutically acceptable salt thereof, is selected from (C₁-C₄)alkyl, phenyl, 4- to 6-membered monocyclic heterocyclyl, 9- or 10-membered fused bicyclic heterocyclyl, 5- or 6-membered monocyclic heteroaryl, and 9- or 10-membered fused bicyclic heteroaryl, wherein said (C₁-C₄)alkyl is optionally substituted with, as valency permits, 1 to 3 groups selected from R^{Y} and said phenyl, 5- or 6-membered monocyclic heterocyclyl, 9- or 10-membered fused bicyclic heterocyclyl, 5- or 6-membered monocyclic heteroaryl, and 9- or 10-membered fused bicyclic heteroaryl are each optionally substituted with, as valency permits, 1 to 3 groups selected from R^{Z}, wherein the remaining variables are as described above for Formula I or any one of the second, fourth to eighth, and tenth to fourteenth embodiments. Alternatively, as part of a fifteenth embodiment, R⁷ in the compound of structural Formula I, II, III, IV, V, VI, VII, or VIII, or a pharmaceutically acceptable salt thereof, is selected from (C₁-C₄)alkyl, phenyl, pyridinyl, pyrimidinyl, naphthyl, cinnolinyl, quinoxalinyl, quinazolinyl, quinolinyl, isoquinolinyl, chromanyl, pyrazoyl, indazolyl, benzoisoxazolyl, imidazo[1,2-a]pyridinyl, pyrrolidinyl, cyclopentyl, cyclohexyl, azetidinyl, piperidinyl, and dihydropyridinyl, and wherein said (C₁-C₄)alkyl is optionally substituted with, as valency permits, 1 to 3 groups selected from R^{Y} and said phenyl, pyridinyl, pyrimidinyl, naphthyl, cinnolinyl, quinoxalinyl, quinazolinyl, quinolinyl, isoquinolinyl, chromanyl, pyrazoyl, indazolyl, benzoisoxazolyl, imidazo[1,2-a]pyridinyl, pyrrolidinyl, cyclopentyl, cyclohexyl, azetidinyl, piperidinyl, and dihydropyridinyl, are each optionally substituted with, as valency permits, 1 to 3 groups selected from R^{Z}, wherein the remaining variables are as described above for Formula I or any one of the second, fourth to eighth, and tenth to fourteenth embodiments.

In a sixteenth embodiment, R^{Y} in the compound of structural Formula I, II, III, IV, V, VI, VII, or VIII, or a pharmaceutically acceptable salt thereof, is selected from cyano, (C₁-C₄)alkyl, —NR^{a}R^{b}, and hydroxyl, wherein the remaining variables are as described above for Formula I or any one of the second, fourth to eighth, and tenth to fifteenth embodiments.

In a seventeenth embodiment, R^{Z} in the compound of structural Formula I, II, III, IV, V, VI, VII, or VIII, or a pharmaceutically acceptable salt thereof, is selected from halo, cyano, (C₁-C₄)alkyl, halo(C₁-C₄)alkyl, hydroxy(C₁-C₄)alkyl, —(C₁-C₄)alkylphenyl, —(C₁-C₄)alkylheteroaryl, (C₁-C₄)alkoxy, halo(C₁-C₄)alkoxy, —OR^{e}, oxo, phenyl, 4- to 6-membered heterocyclyl, —S(O)₂R^{f}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, and wherein said phenyl and said 4- to 6-membered heterocyclyl are each optionally and independently substituted with, as valency permits, 1 to 3 groups selected from cyano and oxo, wherein the remaining variables are as described above for Formula I or any one of the second, fourth to eighth, and tenth to sixteenth embodiments. Alternatively, as part of a seventeenth embodiment, R^{Z} in the compound of structural Formula I, II, III, IV, V, VI, VII, or VIII, or a pharmaceutically acceptable salt thereof, is selected halo, cyano, (C₁-C₄)alkyl, halo(C₁-C₄)alkyl, hydroxy(C₁-C₄)alkyl, —(C₁-C₄)alkylphenyl, —(C₁-C₄)alkylheteroaryl, (C₁-C₄)alkoxy, halo(C₁-C₄)alkoxy, —OR^{e}, oxo, —S(O)₂R^{f}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, —NR^{a}R^{b}, phenyl, and dihydropyridinyl, wherein the (C₁-C₄)alkyl is optionally substituted with cyano, and wherein said phenyl and said dihydropyridinyl are each optionally and independently substituted with, as valency permits, 1 to 3 groups selected from cyano and oxo, wherein the remaining variables are as described above for Formula I or any one of the second, fourth to eighth, and tenth to sixteenth embodiments.

In an eighteenth embodiment, R⁶ and R⁷ in the compound of structural Formula I, II, III, IV, V, VI, VII, or VIII, or a pharmaceutically acceptable salt thereof, together with the nitrogen atom to which they are attached form 4- to 6-membered monocyclic heterocyclyl, 7- to 13-membered spiro bicyclic heterocyclyl, or 9- to 10-membered fused bicyclic heterocyclyl, each of which being optionally substituted with, as valency permits, 1 to 3 groups selected from R^{Q}, wherein the remaining variables are as described above for Formula I or any one of the second, fourth to eighth, and tenth to seventeenth embodiments. Alternatively, as part of a eighteenth embodiment, R⁶ and R⁷ in the compound of structural Formula I, II, III, IV, V, VI, VII, or VIII, or a pharmaceutically acceptable salt thereof, together with the nitrogen atom to which they are attached form azetidinyl, spiro[indoline-3,3′-pyrrolidinyl], pyrrolidinyl, 3,4-dihydrobenzooxazinyl, 1,2,3,4-tetrahydroquinolinyl, octahydrothiopyranopyrroyl, octahydro-1H-thieno[3′,4′:3,4]cyclobuta[1,2-c]pyrroyl, 2,6-diazabicyclo[3.2.0]heptanyl, piperazinyl, morpholinyl, 1,4,5,6-tetrahydropyrrolopyrazoyl, 4-azaspiro[2.4]heptanyl, 2-oxa-5-azabicyclo[2.2.1]heptanyl, 7-oxa-4-azaspiro[2.5]octanyl, 8-oxa-3-azabicyclo[3.2.1]octanyl, 4,6-diazaspiro[2.4]heptanyl, 5-oxa-2,6-diazaspiro[3.4]oct-6-enyl, or piperidinyl, each of which being optionally substituted with, as valency permits, 1 to 3 groups selected from R^{Q}, wherein the remaining variables are as described above for Formula I or any one of the second, fourth to eighth, and tenth to seventeenth embodiments. In another alternative, as part of an eighteenth embodiment, R⁶ and R⁷ in the compound of structural Formula I, II, III, IV, V, VI, VII, or VIII or a pharmaceutically acceptable salt thereof, together with the nitrogen atom to which they are attached form pyrrolidinyl or azetidinyl, each of which being optionally substituted with, as valency permits, 1 to 3 groups selected from R^{Q}, wherein the remaining variables are as described above for Formula I or any one of the second, fourth to eighth, and tenth to seventeenth embodiments.

In a nineteenth embodiment, R^{Q} in the compound of structural Formula I, II, III, IV, V, VI, VII, or VIII, or a pharmaceutically acceptable salt thereof, is selected from halo, (C₁-C₄)alkyl, halo(C₁-C₄)alkyl, cyano, phenyl, 4- to 9-membered monocyclic or bicyclic heterocyclyl, 5- to 10-membered monocyclic or bicyclic heteroaryl, (C₃-C₆)cycloalkyl, oxo, —OR^{e}, —C(O)R^{g}, —C(O)NR^{c}R^{d}, —C(O)OR^{e}, —NR^{c}C(O)R^{e}, wherein said (C₁-C₄)alkyl are each optionally and independently substituted with, as valency permits, 1 to 3 groups selected from R^{M}, and wherein said phenyl, 4- to 6-membered heterocyclyl, 5- to 10-membered monocyclic or bicyclic heteroaryl, and (C₃-C₆)cycloalkyl are each optionally and independently substituted with, as valency permits, 1 to 3 groups selected from R^{F}, wherein the remaining variables are as described above for Formula I or any one of the second, fourth to eighth, and tenth to eighteenth embodiments. Alternatively, as part of a nineteenth embodiment, R^{Q} in the compound of structural Formula I, II, III, IV, V, VI, VII, or VIII, or a pharmaceutically acceptable salt thereof, is selected from cyano, (C₁-C₄)alkyl, halo(C₁-C₄)alkyl, oxo, —C(O)R^{g}, —OR^{e}, —C(O)OR^{e}, —C(O)NR^{c}R^{d}, —NR^{c}C(O)R^{e}, cyclobutyl, cyclopropyl, cyclohexyl, phenyl, pyridinyl, pyrimidinyl, dihydropyridinyl, pyrazoyl, piperidinyl, tetrahydroimidazopyridinyl, and isothiazolidinyl, wherein said (C₁-C₄)alkyl is optionally and independently substituted with, as valency permits, 1 to 3 groups selected from R^{M}, and wherein said cyclobutyl, cyclopropyl, cyclohexyl, phenyl, pyridinyl, pyrimidinyl, dihydropyridinyl, pyrazoyl, piperidinyl, tetrahydroimidazopyridinyl, and isothiazolidinyl are each optionally and independently substituted with, as valency permits, 1 to 3 groups selected from R^{F}, wherein the remaining variables are as described above for Formula I or any one of the second, fourth to eighth, and tenth to eighteenth embodiments.

In a twentieth embodiment, R^{M} in the compound of structural Formula I, II, III, IV, V, VI, VII, or VIII, or a pharmaceutically acceptable salt thereof, is selected from phenyl and 5- to 6-membered monocyclic heteroaryl wherein said phenyl and 5- to 6-membered monocyclic heteroaryl, and phenyl are each optionally substituted with, as valency permits, 1 to 3 groups selected from R^{X}, wherein the remaining variables are as described above for Formula I or any one of the second, fourth to eighth, and tenth to nineteenth embodiments. Alternatively, as part of a twentieth embodiment, R^{M} in the compound of structural Formula I, II, III, IV, V, VI, VII, or VIII, or a pharmaceutically acceptable salt thereof, is selected from phenyl and pyridinyl, wherein said phenyl and pyridinyl are each optionally substituted with, as valency permits, 1 to 3 groups selected from R^{X}, wherein the remaining variables are as described above for Formula I or any one of the second, fourth to eighth, and tenth to nineteenth embodiments.

In a twenty-first embodiment, R^{X} in the compound of structural Formula I, II, III, IV, V, VI, VII, or VIII, or a pharmaceutically acceptable salt thereof, is selected from halo and (C₁-C₄)alkoxy, wherein the remaining variables are as described above for Formula I or any one of the second, fourth to eighth, and tenth to twentieth embodiments.

In a twenty-second embodiment, R^{F} in the compound of structural Formula I, II, III, IV, V, VI, VII, or VIII, or a pharmaceutically acceptable salt thereof, is selected from halo, cyano, (C₁-C₄)alkoxy, oxo, and —NR^{c}C(O)R^{e}, wherein the remaining variables are as described above for Formula I or any one of the second, fourth to eighth, and tenth to twenty-first embodiments.

In a twenty-third embodiment, R^{c} and R^{d} in the compound of structural Formula I, II, III, IV, V, VI, VII, or VIII, or a pharmaceutically acceptable salt thereof, are each independently selected from, as valency permits, hydrogen and (C₁-C₄)alkyl, and wherein said (C₁-C₄)alkyl is optionally substituted with, as valency permits, 1 to 3 groups selected from RJ, wherein the remaining variables are as described above for Formula I or any one of the second, fourth to eighth, and tenth to twenty-second embodiments.

In a twenty-fourth embodiment, R^{e} in the compound of structural Formula I, II, III, IV, V, VI, VII, or VIII, or a pharmaceutically acceptable salt thereof, is independently selected from hydrogen, phenyl, 5- to 6-membered heteroaryl, and (C₁-C₄)alkyl, and wherein said (C₁-C₄)alkyl is optionally substituted with, as valency permits, 1 to 3 groups selected from RJ, wherein the remaining variables are as described above for Formula I or any one of the second, fourth to eighth, and tenth to twenty-third embodiments.

In a twenty-fifth embodiment, R^{J} in the compound of structural Formula I, II, III, IV, V, VI, VII, or VIII, or a pharmaceutically acceptable salt thereof, is independently selected from phenyl and 5- to 6-membered heteroaryl, and wherein the 5- to 6-membered heteroaryl is optionally substituted with a (C₃-C₆)cycloalkyl, wherein the remaining variables are as described above for Formula I or any one of the second, fourth to eighth, and tenth to twenty-fourth embodiments. Alternatively, as part of a twenty-fifth embodiment, RJ in the compound of structural Formula I, II, III, IV, V, VI, VII, or VIII, or a pharmaceutically acceptable salt thereof, is phenyl, wherein the remaining variables are as described above for Formula I or any one of the second, fourth to eighth, and tenth to twenty-fourth embodiments.

In a twenty-sixth embodiment, R^{g} in the compound of structural Formula I, II, III, IV, V, VI, VII, or VIII, or a pharmaceutically acceptable salt thereof is selected from piperidinyl, morpholinyl, and piperizinyl, wherein the remaining variables are as described above for Formula I or any one of the second, fourth to eighth, and tenth to twenty-fifth embodiments.

In a twenty-seventh embodiment, R⁸ in the compound of structural Formula I, II, III, VI, or VIII, or a pharmaceutically acceptable salt thereof is hydrogen, —C(O)R^{Ha}, —C(O)OR^{Ha}, (C₁-C₄)alkyl, —SO₂R^{Ha}, —C(O)NR^{Ha}R^{Hb}, halo(C₁-C₄)alkyl, phenyl, —(C₁-C₄)[4- to 10-membered monocyclic or bicyclic heterocyclyl], —(C₁-C₄)[5- to 10-membered monocyclic or bicyclicheteroaryl], —(C₃-C₆)cycloalkyl, or 4- to 10-membered monocyclic or bicyclic heterocyclyl, wherein the phenyl on the —(C₁-C₄)alkylphenyl is optionally substituted with 1 or 2 groups independently selected from —(C₁₋₃alkoxy) and 5- to 6-membered heteroaryl optionally substituted with —(C₁-C₄)alkyl, and wherein the (C₁-C₄)alkyl is optionally substituted with 1 to 3 phenyl; R^{Ha} is (C₁-C₄)alkyl, —(C₁-C₄)[5- to 6-membered heteroaryl], —(C₁-C₄)[phenyl], —(C₁-C₄)[(C₃-C₆)cycloalkyl], phenyl, 4- to 10-membered monocyclic or bicyclic heterocyclyl, wherein the phenyl and 4- to 10-membered monocyclic or bicyclic heterocyclyl are each optionally substituted with 1 to 3 groups independently selected from halo, (C₁-C₄)alkyl, (C₁-C₄)alkoxy, and 4- to 6-membered monocyclic heterocyclyl optionally substituted with C₁-C₄alkyl; and R^{Hb} is H, wherein the remaining variables are as described above for Formula I or any one of the second, fourth to eighth, and tenth to twenty-sixth embodiments. Alternatively, as part of a twenty-seventh embodiment, R⁸ in the compound of structural Formula I, II, III, VI, or VIII, or a pharmaceutically acceptable salt thereof is —H, —CH₂CH₃, —CH₂CH(CH₃)₂, —CH₂CF₂, —CH₂CF₃, cyclobutyl, —CH₂-quinuclidinyl, —CH₂-imidazo[1,5-a]pyridinyl, —CH₂-pyrimidinyl, —CH₂-phenyl, phenyl, pyrimidinyl, pyridinyl, isoquinolinyl, —C(O)R^{Ha}, —C(O)OR^{Ha}, —SO₂R^{Ha}, —C(O)NR^{Ha}R^{Hb}, or R⁸ is represented by the following structure:

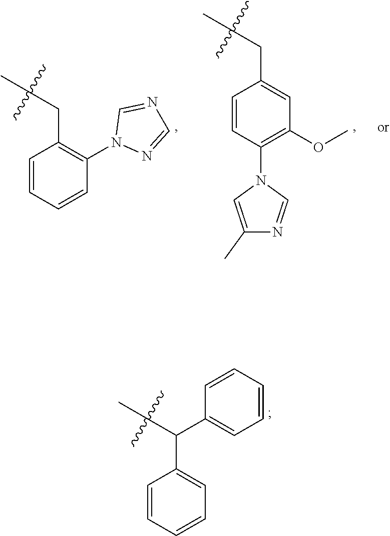

and R^{Ha} is methyl, ethyl, propyl, isopropyl, —CH₂-quinolinyl, —CH₂-phenyl, —CH₂CH₂-phenyl, —CH(CH₃)-phenyl, —CH(CH₃)CH₂-phenyl, —CH₂CH(CH₃)-phenyl, —CH₂CH₂-pyrimidinyl, —CH₂C(CH₃)₂-cyclohexyl, quinolinyl, indazoyl, benzoisoxazolyl, imidazo[1,5-a]pyridinyl, pyrazolo[1,5-a]pyrimidinyl, 5,6,7,8-tetrahydroimidazo[1,5-a]pyridinyl, 4,5,6,7-tetrahydrobenzo[d]isoxazolyl, or 6,7-dihydro-4H-pyrano[3,4-d]isoxazolyl, wherein said quinolinyl, indazoyl, benzoisoxazolyl, imidazo[1,5-a]pyridinyl, pyrazolo[1,5-a]pyrimidinyl, 5,6,7,8-tetrahydroimidazo[1,5-a]pyridinyl, 4,5,6,7-tetrahydrobenzo[d]isoxazolyl, 6,7-dihydro-4H-pyrano[3,4-d]isoxazolyl are each optionally substituted with 1 to 3 groups independently selected from —Cl, —CH₃, —CH₂CH₃, —OCH₃, or N-methylpiperazinyl, wherein the remaining variables are as described above for Formula I or any one of the second, fourth to eighth, and tenth to twenty-sixth embodiments.

Compounds having the Formula I are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of STAT proteins, in particular STAT3 and/or STAT6. In some aspects, the compounds, pharmaceutical acceptable salts, and pharmaceutical compositions described herein inhibit the activity STAT3 and/or STAT6.

In some aspects, the compounds and pharmaceutical compositions described herein are useful in a condition responsive to the modulation of STAT3 and/or STAT6. Thus, provided herein are methods of treating a condition responsive to the modulation (e.g., inhibition) of STAT3 and/or STAT6 in a subject, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a condition responsive to the modulation (e.g., inhibition) of STAT3 and/or STAT6. Also provided is a compound described herein, or a pharmaceutically acceptable salt thereof, or a pharmaceutical composition comprising a disclosed compound or pharmaceutically acceptable salt thereof, for use in treating a condition responsive to the modulation (e.g., inhibition) of STAT3 and/or STAT6.

In one aspect, the condition responsive to the modulation (e.g., inhibition) of STAT3 and/or STAT6 include, but are not limited to, cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In another aspect, the condition responsive to the modulation (e.g., inhibition) of STAT3 and/or STAT6 include, but are not limited to, cancer (see, e.g., Turkson & Jove, Oncogene 2000, 19:6613-6626), diabetes (see. e.g., Gurzov et al., FEBS 2016, 283:3002), cardiovascular disease (see, e.g., Grote et al., Vasc. Pharmacol. 2005, 43:2005), viral disease (see, e.g., Gao et al., J Hepatol. 2012, 57(2):430), autoimmune diseases such as lupus (see, e.g., Goropevsek et al., Clin. Rev. Alleg. & Immun. 2017, 52(2):164), and rheumatoid arthritis (see, e.g., Walker & Smith, J. Rheumat. 2005, 32(9): 1650), autoinflammatory syndromes (see, e.g., Rauch et al., Jak-Stat 2013, 2(1):e23820), atherosclerosis (see, e.g., Ortiz-Munoz et al., Arterio., Thromho., Vase. Bio. 2009, 29:525), psoriasis (see, e.g., Andres et al., Exp. Derm. 2013, 22(5):323), allergic disorders (see, e.g., Oh et al., Eur. Respir. Rev. 2019, 19(115):46), inflammatory bowel disease (see. e.g., Sugimoto, World J Gastroenterol. 2008, 14(33):5110), inflammation (see, e.g., Tamiya et al., Arierio. Thrombo., Vasc. Bio. 2011, 31:980), acute and chronic gout and gouty arthritis, neurological disorders (see, e.g., Campbell, Brain Res. Rev. 2005, 48(2): 166), metabolic syndrome, immunodeficiency disorders such as AIDS and HIV (see, e.g., O'Shea et al., N. Engl. J. Med. 2013, 368:161), destructive bone disorders (see, e.g., Jatiani et al., Genes & Can. 2011, 1(10):979), osteoarthritis, proliferative disorders, Waldenstrom's Macroglobulinemia (see, e.g., Hodge et al., Blood 2014, 123(7):1055) infectious diseases, conditions associated with cell death, pathologic immune conditions involving T cell activation, and CNS disorders.

Proliferative disorders, include, but are not limited to a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-I driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the cancer to be treated is selected from glioma, breast cancer, prostate cancer, head and neck squamous cell carcinoma, skin melanomas, ovarian cancer, malignant peripheral nerve sheath tumors (MPNST), and pancreatic cancer. In other embodiments, the cancer to be treated is cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes. In other embodiments, the cancer is selected from solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia Such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

Compounds, salts, and compositions described herein are also useful in the treatment of inflammatory or obstructive airways diseases, resulting, for example, in reduction of tissue damage, airways inflammation, bronchial hyperreactivity, remodeling or disease progression. Inflammatory or obstructive airways diseases include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds, salts, and compositions described herein are also useful in the treatment of heteroimmune diseases including, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Compounds, salts, and compositions described herein are also useful in the treatment of other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. Compounds, salts, and compositions described herein are also useful in the treatment of bronchitis including, but not limited to, acute, arachidic, catarrhal, croupus, chronic or phthinoid bronchitis. Compounds, salts, and compositions described herein are also useful in the treatment of pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds, salts, and compositions described herein are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, Pemphigus vulgaris, Pemphigus foliaceus, paraneoplastic pemphigus, epidermolysis bullosa acquisita, acne vulgaris, and other inflammatory or allergic conditions of the skin.

Compounds, salts, and compositions described herein are also useful in the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospirosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, cardiovascular diseases which can be treated according to the present methods include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the present methods include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Preparation of Compounds

The compounds claimed herein were prepared following the procedures outlined in the following schemes. Compound names were generated using the software built into ChemDraw. To the extent that there are discrepancies between the name of a compound and its depicted structure, the depicted chemical structure is to be taken as the appropriate compound.

### Synthesis of Cores

### Synthesis of Methyl (5S,8S,10aR)-5-((tert-butoxycarbonyl)amino)-6-oxodecahydropyrrolo[1,2-a][1,4]diazocine-8-carboxylate

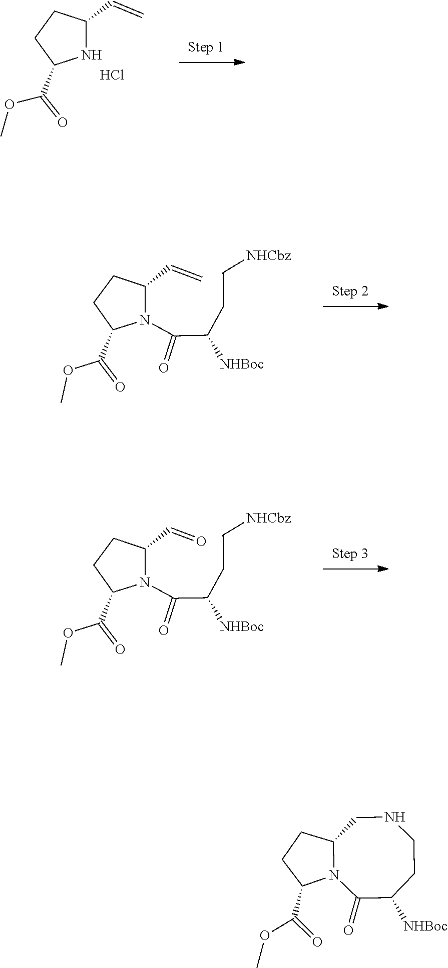 [SMILES: C=C[C@H]1CC[C@@H](C(=O)OC)N1 ; Cl ; C=C[C@H]1CC[C@@H](C(=O)OC)N1C(=O)[C@H](CCNC(=O)OCc1ccccc1)NC(=O)OC(C)(C)C ; COC(=O)[C@@H]1CC[C@H](C=O)N1C(=O)[C@H](CCNC(=O)OCc1ccccc1)NC(=O)OC(C)(C)C ; COC(=O)[C@@H]1CC[C@@H]2CNCC[C@H](NC(=O)OC(C)(C)C)C(=O)N21]

### Step 1: Preparation of Methyl (2S,5R)-1-((S)-4-(((benzyloxy)carbonyl)amino)-2-((tert-butoxycarbonyl)amino)butanoyl)-4-vinylpyrrolidine-2-carboxylate

To a solution of methyl (2S,5R)-5-vinylpyrrolidine-2-carboxylate hydrochloride (12.1 g, 63.1 mmol, 1.00 eq) in DCM (302 mL) was added BOPCl (16.1 g, 63.1 mmol, 1.00 eq), NaHCO₃ (21.2 g, 252 mmol, 9.81 mL, 4.00 eq) and (S)-4-(((benzyloxy)carbonyl)amino)-2-((tert-butoxycarbonyl)amino)butanoic acid (20.0 g, 56.8 mmol, 0.90 eq). The reaction mixture was heated to 40° C. After stirring for 2 h, the reaction mixture was concentrated under reduced pressure. The residue was purified by column chromatography (gradient elution, petroleum ether/EtOAc=1/0 to 0/1) to give methyl (2S,5R)-1-((S)-4-(((benzyloxy)carbonyl)amino)-2-((tert-butoxycarbonyl)amino)butanoyl)-5-vinylpyrrolidine-2-carboxylate (18.3 g, crude) as light-yellow gel. ¹H NMR (400 MHz, CDCl₃) δ 7.43-7.28 (m, 5H), 5.99-5.83 (m, 1H), 5.43 (br d, J=17.0 Hz, 1H), 5.24-5.12 (m, 2H), 5.12-5.03 (m, 2H), 4.77-4.61 (m, 1H), 4.58-4.43 (m, 2H), 3.76-3.69 (m, 3H), 3.56-3.41 (m, 1H), 3.01 (tdd, J=6.0, 9.4, 14.2 Hz, 1H), 2.27-2.13 (m, 2H), 2.02-1.91 (m, 2H), 1.88-1.80 (m, 1H), 1.68-1.55 (m, 2H), 1.42 (s, 9H).

### Step 2: Preparation of Methyl (2S,5R)-1-((S)-4-(((benzyloxy)carbonyl)amino)-2-((tert-butoxycarbonyl)amino)butanoyl)-5-formylpyrrolidine-2-carboxylate

To a cooled (−40° C.) solution of methyl (2S,5R)-1-((S)-4-(((benzyloxy)carbonyl)amino)-2-((tert-butoxycarbonyl)amino)butanoyl)-5-vinylpyrrolidine-2-carboxylate (18.3 g, 37.4 mmol, 1.00 eq) in DCM (183 mL) was bubbled ozone (5 psi). After 1 h, the reaction mixture was sparged N₂ (g) to remove excess ozone, followed by addition of Et₃N (14.1 mL, 101 mmol, 2.71 eq). The reaction mixture was warmed to room temperature and stirred for 1 h. The mixture was concentrated in vacuo and the resulting residue was purified by column chromatography (gradient elution, petroleum ether/EtOAc=0/1 to 1/0) to give methyl (2S,5R)-1-((S)-4-(((benzyloxy)carbonyl)amino)-2-((tert-butoxycarbonyl)amino)butanoyl)-5-formylpyrrolidine-2-carboxylate (13.7 g, 18.6 mmol, 49.7% yield for two steps, 66.6% purity) as light-yellow syrup. LCMS (ESI) m/z=492.2 [M+H]⁺

### Step 3: Preparation of Methyl (5S,8S,10aR)-5-((tert-butoxycarbonyl)amino)-6-oxodecahydropyrrolo[1,2-a][1,4]diazocine-8-carboxylate

To a solution of methyl (2S,5R)-1-((S)-4-(((benzyloxy)carbonyl)amino)-2-((tert-butoxycarbonyl)amino)butanoyl)-5-formylpyrrolidine-2-carboxylate (13.7 g, 18.6 mmol, 66.6% purity, 1.00 eq) in i-PrOH (137 mL) was added Pd/C (6.30 g, 10.0% purity) under N₂ (g). The suspension subjected to three cycles of evacuation and purging with H₂ (g) (50 Psi). The mixture was heated to 40° C. After 12 h, the mixture was purged with N₂ (g). The suspension was filtered and the filtrate was concentrated under reduced pressure. The residue was purified by prep-HPLC (column: Kromasil Eternity XT 250×80 mm×10 um; mobile phase: [water (ammonia hydroxide v/v)-acetonitrile]; B %: 10%-40%, 20 min) and lyophilized to give methyl (5S,8S,10aR)-5-((tert-butoxycarbonyl)amino)-6-oxodecahydropyrrolo[1,2-a][1,4]diazocine-8-carboxylate (3.39 g, 9.38 mmol, 50.5% yield, 94.4% purity) as a white solid. LCMS (ESI) m/z=342.1 [M+H]⁺; ¹H NMR (400 MHz, CDCl₃) δ 5.51 (br d, J=8.0 Hz, 1H), 4.80-4.72 (m, 1H), 4.53 (t, J=8.6 Hz, 1H), 4.30-4.21 (m, 1H), 3.76 (s, 3H), 3.16-3.08 (m, 1H), 2.99-2.84 (m, 3H), 2.34-2.28 (m, 1H), 2.20-2.16 (m, 2H), 2.06-1.89 (m, 2H), 1.77-1.59 (m, 2H), 1.42 (s, 9H).

### Synthesis of (5R,8S,10aR)-5-((tert-butoxycarbonyl)amino)-6-oxooctahydro-4H-pyrrolo[2,1-d][1,5]thiazocine-8-carboxylic Acid

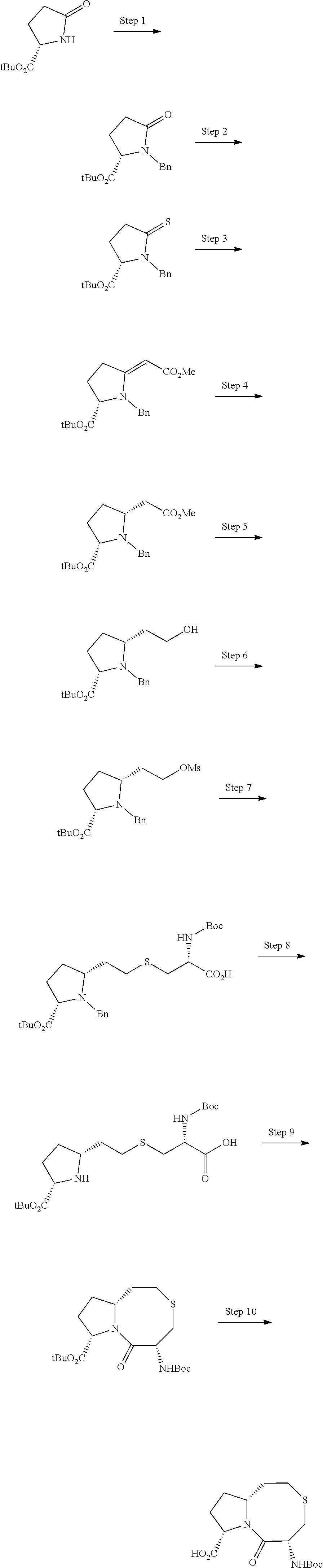

### Step 1: Preparation of Tert-Butyl (S)-1-benzyl-5-oxopyrrolidine-2-carboxylate

To a cooled (0° C.) suspension of NaH (32.4 g, 810 mmol, 60% purity, 1.20 eq) in THF (250 mL) was added a solution of tert-butyl (S)-5-oxopyrrolidine-2-carboxylate (125 g, 675 mmol, 1.00 eq) in THF (1.00 L) in a dropwise manner. The mixture was warmed to 25° C. and stirred for 1 h. Then the mixture was subsequently cooled to 0° C., followed by dropwise addition of benzyl bromide (88.2 mL, 742 mmol, 1.10 eq). After complete addition of benzyl bromide, the reaction mixture was warmed to 25° C. and stirred for 2 h. Four batches of equivalent scale were performed and subsequently combined were combined for workup. The reaction mixture was poured into cooled saturated aqueous NH₄Cl (1.30 L). The biphasic mixture was extracted with EtOAc (500 mL×3) and the organic phases combined. The combined organic layers were washed with brine (800 mL), dried over Na₂SO₄, filtered and concentrated under reduced pressure to give a crude product. The crude product was purified by column chromatography (gradient elution: petroleum ether/EtOAc=1/0 to 50/1) and concentrated under reduced pressure to give tert-butyl (S)-1-benzyl-5-oxopyrrolidine-2-carboxylate (397 g, 1.41 mol, 52.1% yield, 98.0% purity) as a yellow solid. LCMS (ESI) m/z=275.9 [M+H]⁺; ¹H NMR (400 MHz, CDCl₃) δ 7.34-7.28 (m, 3H), 7.23-7.21 (m, 2H), 5.06 (d, J=14.8 Hz, 1H), 3.96 (d, J=14.8 Hz, 1H), 3.85-3.82 (m, 1H), 2.56-2.44 (m, 1H), 2.43-2.41 (m, 1H), 2.24-2.19 (m, 1H), 2.06-2.04 (m, 1H), 1.44 (s, 9H).

### Step 2: Preparation of Tert-Butyl (S)-1-benzyl-5-thioxopyrrolidine-2-carboxylate

To a solution of tert-butyl (S)-1-benzyl-5-oxopyrrolidine-2-carboxylate (179 g, 648 mmol, 1.00 eq) in DCM (1.79 L) was added Lawesson's Reagent (131 g, 324 mmol, 0.50 eq) at 25° C. Two batches of equivalent scale were performed and subsequently combined for workup. After stirring for 4 h, the reaction mixtures were filtered and the filtrate was concentrated under reduced pressure to give a crude product. The crude product was purified by column chromatography (gradient elution: petroleum ether/EtOAc=1/0 to 50/1) to give tert-butyl (S)-1-benzyl-5-thioxopyrrolidine-2-carboxylate (324 g, 1.11 mol, 85.5% yield, 99.7% purity) as a yellow solid. LCMS (ESI) m/z=292.1 [M+H]⁺; ¹H NMR (400 MHz, CDCl₃) δ 7.35-7.30 (m, 5H), 5.81 (d, J=14.4 Hz, 1H), 4.28 (d, J=14.8 Hz, 1H), 4.17-4.14 (m, 1H), 3.16-3.09 (m, 2H), 2.27-2.23 (m, 1H), 2.22-2.10 (m, 1H), 1.45 (s, 9H).

### Step 3: Preparation of Tert-Butyl (S,Z)-1-benzyl-5-(2-methoxy-2-oxoethylidene)pyrrolidine-2-carboxylate

A solution of tert-butyl (S)-1-benzyl-5-thioxopyrrolidine-2-carboxylate (58.0 g, 199 mmol, 1.00 eq) and methyl bromoacetate (37.7 g, 247 mmol, 23.3 mL, 1.24 eq) in acetonitrile (160 mL) was stirred at 25° C. After stirring for 40 h, the reaction mixture was diluted with dichloromethane (1.20 L) and cooled to 0° C. After an additional 10 min, PPh₃ (78.3 g, 298 mmol, 1.50 eq) and Et₃N (83.1 mL, 597 mmol, 3.00 eq) were sequentially added. The reaction mixture was allowed to warm to ambient temperatures and stirred for 16 hr. Six batches of equivalent scale were performed and subsequently combined for workup. The mixtures were poured into aqueous 1 N NaH₂PO₄ (2.00 L). The biphasic mixture was extracted with dichloromethane (800 mL×3). The combined organic layer was washed with brine (800 mL), dried over Na₂SO₄, filtered, and concentrated to give residue. The residue was purified by column chromatography (gradient elution: petroleum ether/EtOAc=80/1 to 15/1) to give tert-butyl (S,Z)-1-benzyl-5-(2-methoxy-2-oxoethylidene)pyrrolidine-2-carboxylate (255 g, 706 mmol, 59.2% yield, 91.8% purity) as yellow oil. LCMS (ESI) m/z=332.1 [M+H]⁺; ¹H NMR (400 MHz, CDCl₃) δ 7.35-7.29 (m, 3H), 7.20-7.18 (m, 2H), 4.76 (s, 1H), 4.54 (d, J=15.6 Hz, 1H), 4.20 (d, J=15.6 Hz, 1H), 3.98-3.95 (m, 1H), 3.75 (s, 3H), 3.62-3.40 (m, 1H), 3.11-3.09 (m, 1H), 2.25-2.20 (m, 1H), 2.10-2.05 (m, 1H), 1.44 (s, 9H).

### Step 4: Preparation of Tert-Butyl (2S,5R)-1-benzyl-5-(2-methoxy-2-oxoethyl)pyrrolidine-2-carboxylate

To a suspension of tert-butyl (S,Z)-1-benzyl-5-(2-methoxy-2-oxoethylidene)pyrrolidine-2-carboxylate (125 g, 377 mmol, 1.00 eq) in EtOAc (1.25 L) was added 10% Pd/C (125 g) under N₂ (g). The suspension was subjected to three cycles of evacuation and purging with H₂ (g) (50 psi). The mixture was stirred at 25° C. under H₂ (g) (50 psi). After stirring fro 12 h, the mixture was subjected to three cycles of evacuation and purging with N₂ (g) and filtered over a pad of Celite®. The filtrate was transferred to another vessel and Pt/C (125 g, 5% purity) was added under a stream of N₂ (g). The suspension was to three cycles of evacuation and purging with H₂ (g) (50 psi). The reaction mixture was heated to 60° C. and stirred at under H₂ (50 Psi) for 16 h. Two batches of equivalent scale were performed and subsequently combined for workup. The combined reaction mixtures were filtered and the filtrate was concentrated in vacuo. The residue was purified by column chromatography (gradient elution: petroleum ether/EtOAc=80/1 to 30/1) to give tert-butyl (2S,5R)-1-benzyl-5-(2-methoxy-2-oxoethyl)pyrrolidine-2-carboxylate (119 g, 358 mmol, 47.5% yield, 100% purity) as yellow oil. LCMS (ESI) m/z=334.1 [M+H]⁺; ¹H NMR (400 MHz, CDCl₃) δ 7.33-7.30 (m, 4H), 7.24-7.22 (m, 1H), 3.89-3.78 (m, 2H), 3.63 (s, 3H), 3.29-3.25 (m, 2H), 2.60-2.55 (m, 1H), 2.37-2.31 (m, 1H), 2.05-2.00 (m, 2H), 1.99-1.88 (m, 1H), 1.68-1.66 (m, 1H), 1.45 (s, 9H).

### Step 5: Preparation of Tert-Butyl (2S,5R)-1-benzyl-5-(2-hydroxyethyl)pyrrolidine-2-carboxylate

To a cooled (0° C.) solution of tert-butyl (2S,5R)-1-benzyl-5-(2-methoxy-2-oxoethyl)pyrrolidine-2-carboxylate (58.5 g, 175 mmol, 1.00 eq) in THF (585 mL) was added LiBH₄ (2.00 M, 175 mL, 2.00 eq) under N₂ (g). The reaction mixture was warmed to 25° C. and stirred for 16 h. Two batches of equivalent scale were performed and subsequently combined for workup. The combined reaction mixture was poured into aqueous 1 N K₂CO₃ (1.20 L) and extracted with EtOAc (800 mL×3). The combined organic layer was washed with brine (1.00 L), dried over Na₂SO₄, filtered, and concentrated to give residue. The residue was purified by column chromatography (gradient elution: petroleum ether/EtOAc=80/1 to 10/1) to give tert-butyl (2S,5R)-1-benzyl-5-(2-hydroxyethyl)pyrrolidine-2-carboxylate (60.0 g, 196 mmol) as yellow oil. LCMS (ESI) m/z=306.1 [M+H]⁺; ¹H NMR (400 MHz, CDCl₃) δ 7.33-7.28 (m, 4H), 7.26-7.24 (m, 1H), 4.09-4.00 (m, 2H), 3.76-3.73 (m, 1H), 3.58-3.55 (m, 1H), 3.30-3.28 (m, 1H), 3.27-3.21 (m, 1H), 2.08-2.04 (m, 2H), 1.91-1.87 (m, 3H), 1.55-1.54 (m, 1H), 1.50 (s, 9H).

### Step 6: Preparation of Tert-Butyl (2S,5R)-1-benzyl-5-(2-((methylsulfonyl)oxy)ethyl)pyrrolidine-2-carboxylate

To a cooled (−10° C.) solution of tert-butyl (2S,5R)-1-benzyl-5-(2-hydroxyethyl)pyrrolidine-2-carboxylate (29.5 g, 96.6 mmol, 1.00 eq) in DCM (295 mL) was added methanesulfonyl chloride (11.7 mL, 151 mmol, 1.57 eq), DMAP (590 mg, 4.83 mmol, 0.05 eq) and Et₃N (40.3 mL, 290 mmol, 3.00 eq). The mixture was warmed to 25° C. and stirred for 1 h. Two batches of equivalent scale were performed and subsequently combined for workup. The reaction mixtures were combined and diluted with dichloromethane (800 mL). The resulting mixture was washed with 0.5 N HCl (1.00 L), washed with aqueous saturated NaHCO₃ (1.00 L) washed with brine (1.00 L), dried over Na₂SO₄, filtered and concentrated under reduced pressure to give tert-butyl (2S,5R)-1-benzyl-5-(2-((methylsulfonyl)oxy)ethyl)pyrrolidine-2-carboxylate (72.0 g, crude) as yellow oil. LCMS (ESI) m/z=384.1 [M+H]⁺

### Step 7: Preparation of S-(2-((2R,5S)-1-benzyl-5-(tert-butoxycarbonyl)pyrrolidin-2-yl)ethyl)-N-(tert-butoxycarbonyl)-L-cysteine

To a cooled (0° C.) solution of tert-butyl (2S,5R)-1-benzyl-5-(2-((methylsulfonyl)oxy)ethyl)pyrrolidine-2-carboxylate (20.8 g, 93.9 mmol, 1.00 eq) in DMF (360 mL) was added NaH (7.51 g, 187 mmol, 60% purity, 2.00 eq) in portions under N₂ (g). The mixture was stirred at 0° C. for 1 h, followed by addition of a solution of (tert-butoxycarbonyl)-L-cysteine (36.0 g, 93.9 mmol, 1.00 eq) in DMF (360 mL). The mixture was warmed to 25° C. and stirred for 1 h. Two batches of equivalent scale were performed and subsequently combined for workup. The reaction mixtures were combined, and the pH of the mixture was adjusted with aqueous 1 N HCl (until pH˜2). The acidic mixture was extracted with EtOAc (2.00 L×2). The combined organic layers were washed with brine (2.00 L), dried over Na₂SO₄, filtered and concentrated under reduced pressure. The residue was purified by column chromatography (gradient elution; petroleum ether/EtOAc=20/1 to 0/1) to give S-(2-((2R,5S)-1-benzyl-5-(tert-butoxycarbonyl)pyrrolidin-2-yl)ethyl)-N-(tert-butoxycarbonyl)-L-cysteine (92.0 g, crude) as yellow oil. LCMS (ESI) m/z=509.2 [M+H]⁺

### Step 8: Preparation of N-(tert-butoxycarbonyl)-5-(2-((2R,5S)-5-(tert-butoxycarbonyl)pyrrolidin-2-yl)ethyl)-L-cysteine

To a solution of S-(2-((2R,5S)-1-benzyl-5-(tert-butoxycarbonyl)pyrrolidin-2-yl)ethyl)-N-(tert-butoxycarbonyl)-L-cysteine (46.0 g, 90.4 mmol, 1.00 eq) in MeOH (460 mL) was added Raney-Ni (46.0 g) under N₂ (g). The suspension was subjected to three cycles of evacuation and purging with H₂ (g). The mixture was stirred at 60° C. under H₂ (g) (50 Psi). After stirring for 16 h, The mixture was purged with N₂ (g) and filtered. The filtrate was transferred to a reaction vessel and Raney-Ni (92.0 g) was added to the filtrate under N₂ (g). The suspension was subjected to three cycles of evacuation and purging with H₂ (g). The mixture was heated to 80° C. and stirred at 80° C. under H₂ (g) (50 Psi) for 40 hr. LCMS analysis showed ˜54.0% of S-(2-((2R,5S)-1-benzyl-5-(tert-butoxycarbonyl)pyrrolidin-2-yl)ethyl)-N-(tert-butoxycarbonyl)-L-cysteine (Rt=0.582 min) was remained and the desired MS (Rt=0.487 min) was detected. The mixture was filtered and Raney-Ni (46.0 g) was added to the filtrate under N₂ (g). The suspension was degassed under vacuum and purged with H₂ several times. The mixture was heated to 80° C. and stirred at 80° C. under H₂ (50 Psi) for 40 hr. LCMS analysis showed 39.0% of S-(2-((2R,5S)-1-benzyl-5-(tert-butoxycarbonyl)pyrrolidin-2-yl)ethyl)-N-(tert-butoxycarbonyl)-L-cysteine (Rt=0.584 min) was remained and desired MS (Rt=0.492 min) was detected. Two batches of equivalent scale were performed and subsequently combined for workup. The reaction mixtures was filtered and the filtrate was combined and concentrated to give residue to give N-(tert-butoxycarbonyl)-S-(2-((2R,5S)-5-(tert-butoxycarbonyl)pyrrolidin-2-yl)ethyl)-L-cysteine (59.0 g, crude) as yellow solid. LCMS (ESI) m/z=419.2 [M+H]⁺.

### Step 9: Preparation of Tert-Butyl (5R,8S,10aR)-5-((tert-butoxycarbonyl)amino)-6-oxooctahydro-4H-pyrrolo[2,1-d][1,5]thiazocine-8-carboxylate

To a cooled (0° C.) solution of N-(tert-butoxycarbonyl)-S-(2-((2R,5S)-5-(tert-butoxycarbonyl)pyrrolidin-2-yl)ethyl)-L-cysteine (14.5 g, 34.6 mmol, 1.00 eq) in DCM (1.50 L) was added PyBOP (54.1 g, 104 mmol, 3.00 eq) and DIPEA (24.9 g, 192 mmol, 33.5 mL, 5.55 eq). The mixture was subsequently warmed to 25° C. and stirred 3 h. Four batches of equivalent scale were performed and subsequently combined for workup. The reaction mixtures were poured into aqueous 0.5 N HCl (4.50 L) and extracted with dichloromethane (2.00 L×2). The combined organic layers were washed with aqueous saturated NaHCO₃ solution (4.50 L). The aqueous layer was extracted with DCM (2.00 L×2). The combined organic layers were washed with brine (1.00 L), dried over Na₂SO₄, filtered and concentrated under reduced pressure. The residue was purified by column chromatography (gradient elution: petroleum ether/EtOAc=1/0˜20/1) and concentrated under reduced pressure to give tert-butyl (5R,8S,10aR)-5-((tert-butoxycarbonyl)amino)-6-oxooctahydro-4H-pyrrolo[2,1-d][1,5]thiazocine-8-carboxylate (12.6 g, 31.1 mmol, 22.4% yield, 98.8% purity) as a white solid. LCMS (ESI) m/z=401.1 [M+H]⁺; ¹H NMR (400 MHz, CDCl₃) δ 5.98-5.96 (m, 1H), 4.69-4.68 (m, 1H), 4.66-4.58 (m, 1H), 4.40-4.35 (m, 1H), 2.94-2.90 (m, 2H), 2.76-2.74 (m, 2H), 2.73-2.74 (m, 1H), 1.99-1.96 (m, 3H), 1.71-1.67 (m, 2H), 1.48-1.42 (m, 18H).

### Step 10: Preparation of (5R,8S,10aR)-5-((tert-butoxycarbonyl)amino)-6-oxooctahydro-4H-pyrrolo[2,1-d][1,5]thiazocine-8-carboxylic Acid

To a cooled (0° C.) solution of tert-butyl (5R,8S,10aR)-5-((tert-butoxycarbonyl)amino)-6-oxooctahydro-4H-pyrrolo[2,1-d][1,5]thiazocine-8-carboxylate (6.30 g, 15.7 mmol, 1.00 eq) in DCM (64.0 mL) was added tri-isopropylsilane (323 uL, 1.57 mmol, 0.10 eq), followed by addition of a solution of TFA (143 mL, 1.93 mol, 123 eq) in DCM (64.0 mL). After stirring for 1 h, the reaction mixture was concentrated to give oil. The resulting amino acid (oil) was diluted in a mixture of acetonitrile (176 mL) and H₂O (8.80 mL), followed by addition of Et₃N (17.7 mL, 127 mmol, 8.10 eq) and Boc₂O (10.3 g, 47.2 mmol, 10.8 mL, 3.00 eq) at 0° C. The mixture was warmed to 25° C. and for 2 h. Two batches of equivalent scale were performed and subsequently combined for workup. The reaction mixtures were combined and concentrated under reduced pressure. The resulting residue was diluted with H₂O (80.0 mL) and extracted with EtOAc (60.0 mL×2). The aqueous layer was adjusted to pH˜2 with aqueous 1 N HCl and extracted with EtOAc (50.0 mL×3). The combined organic layers were washed with brine (40.0 mL×2), dried over Na₂SO₄, filtered, and concentrated under reduced pressure to give a residue. The residue was diluted with H₂O (20.0 mL) and acetonitrile (6.00 mL) and lyophilized to give (5R,8S,10aR)-5-((tert-butoxycarbonyl)amino)-6-oxooctahydro-4H-pyrrolo[2,1-d][1,5]thiazocine-8-carboxylic acid (5.50 g, 15.4 mmol, 48.9% yield, 96.4% purity) as a white solid. LCMS (ESI) m/z=367.1 [M+Na]⁺; ¹H NMR (400 MHz, CDCl₃) δ 6.10 (brs, 1H), 5.87-5.85 (m, 1H), 4.73-4.70 (m, 2H), 4.68-4.58 (m, 1H), 2.98-2.94 (m, 2H), 2.89-2.74 (m, 1H), 2.72-2.71 (m, 1H), 2.54-2.38 (m, 2H), 2.05-2.02 (m, 1H), 1.89-1.86 (m, 2H), 1.75-1.48 (m, 1H), 1.44 (s, 9H).

### (5S,8S,10aR)-3-acetyl-5-((tert-butoxycarbonyl)amino)-6-oxodecahydropyrrolo[1,2-a][1,5]diazocine-8-carboxylic Acid

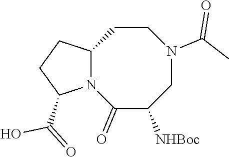 [SMILES: CC(=O)N1CC[C@H]2CC[C@@H](C(=O)O)N2C(=O)[C@@H](NC(=O)OC(C)(C)C)C1]

This is commercially available: CAS 2497590-06-2

### Synthesis of Linkers

### Procedures for Syntheses of Linkers:

The following intermediates in Table 1 were prepared according to the protocol described in WO 2020/205467.

TABLE 1

| Name | Structure |
|---|---|
| 5- ((diethoxyphosphoryl)difluoromethyl)benzo[b] thiophene-2-carboxylic acid | 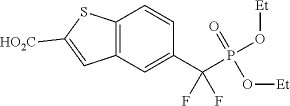 [SMILES: CCOP(=O)(OCC)C(F)(F)c1ccc2sc(C(=O)O)cc2c1] |
| 7-((diethoxyphosphoryl)difluoromethyl)-2- naphthoic acid | 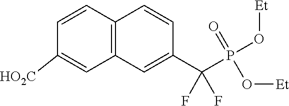 [SMILES: CCOP(=O)(OCC)C(F)(F)c1ccc2ccc(C(=O)O)cc2c1] |
| 5-((diethoxyphosphoryl)difluoromethyl)-1H- indole-2-carboxylic acid | 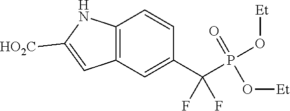 [SMILES: CCOP(=O)(OCC)C(F)(F)c1ccc2[nH]c(C(=O)O)cc2c1] |

### Synthesis of (E)-3-(4-((diethoxyphosphoryl)difluoromethyl)phenyl)acrylic Acid

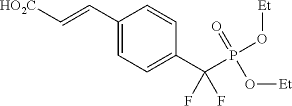 [SMILES: CCOP(=O)(OCC)C(F)(F)c1ccc(/C=C/C(=O)O)cc1]

(E)-3-(4-((diethoxyphosphoryl)difluoromethyl)phenyl)acrylic acid was prepared according to protocol described in US 2004/0225146.

### Representative Procedure for Preparation of Activated Ester Phosphonic Acids

### Synthesis of (difluoro(2-((4-nitrophenoxy)carbonyl)benzo[b]thiophen-5-yl)methyl)phosphonic Acid

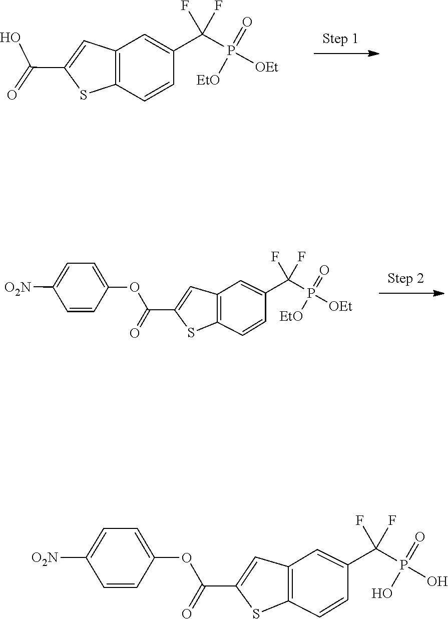 [SMILES: CCOP(=O)(OCC)C(F)(F)c1ccc2sc(C(=O)O)cc2c1 ; CCOP(=O)(OCC)C(F)(F)c1ccc2sc(C(=O)Oc3ccc([N+](=O)[O-])cc3)cc2c1 ; O=C(Oc1ccc([N+](=O)[O-])cc1)c1cc2cc(C(F)(F)P(=O)(O)O)ccc2s1]

### Step 1: Preparation of 4-nitrophenyl 5-((diethoxyphosphoryl)difluoromethyl)benzo[b]thiophene-2-carboxylate

To a mixture of 5-[(diethoxyphosphoryl)difluoromethyl]-1-benzothiophene-2-carboxylic acid (L1) (10.0 g, 27.4 mmol), EDCI (7.85 g, 41.0 mmol) and DMAP (836 mg, 6.85 mmol) in CH₂Cl₂ (80 mL) was stirred at room temperature. After 15 min, 4-nitrophenol (4.75 g, 34.2 mmol) was added and the resulting yellow mixture was stirred at room temperature for 18 h. The reaction was quenched with water (30 mL) and the product was extracted with CH₂Cl₂ (10 mL×2). The combined organic extracts were washed with brine, dried with sodium sulfate, filtered and concentrated in vacuo. The crude residue was purified by reverse phase chromatography [C18 cartridge eluting with a gradient of 5-100% acetonitrile in water] and the appropriate fractions were concentrated to give 4-nitrophenyl 5-((diethoxyphosphoryl)difluoromethyl)benzo[b]thiophene-2-carboxylate (7.80 g, 16.0 mmol, 59.0% yield) as a yellow solid. LCMS m/z=486.2 [M+H]⁺; ¹H NMR (400 MHz, DMSO-d₆) δ 8.32-8.37 (m, 3H), 8.22 (s, 1H), 8.01 (d, J=9.1 Hz, 1H), 7.77 (d, J=7.8 Hz, 1H), 7.51-7.45 (m, 2H), 4.14-4.32 (m, 4H), 1.34 (t, J=7.8 Hz, 6H).

### Step 2: Preparation of (difluoro(2-((4-nitrophenoxy)carbonyl)benzo[b]thiophen-5-yl)methyl)phosphonic Acid

To a cooled (0° C.) solution of 4-nitrophenyl 5-((diethoxyphosphoryl)difluoromethyl)benzo[b]thiophene-2-carboxylate (4.47 g, 9.20 mmol) in CH₂Cl₂ (39 mL) was added N,O-bis(trimethylsilyl)trifluoroacetamide (12.1 mL, 46.0 mmol) and iodotrimethylsilane (5.23 mL, 36.8 mmol) as a solution in CH₂Cl₂ (10 mL). The reaction mixture was gradually allowed to warm to ambient temperatures. To the reaction mixture was added a mixture of 2:1 H₂O/acetonitrile (with 0.1% TFA) (50 mL) and precipitation of product was observed. The volatiles were removed in vacuo and the crude residue was suspended in a mixture of acetonitrile/water solution (1:1 v/v, 100 mL). The suspension was filtered, the solids were washed with a 2:1 mixture acetonitrile/water solution, and the solid were dried under reduced pressure to afford [difluoro({2-[(4-nitrophenoxy)carbonyl]-1-benzothiophen-5-yl})methyl]phosphonic acid (6.5 g, 94%) as a beige solid. The filtrate was concentrated to 50% of solvent volume and the resulting suspension was filtered and washed with 1:2 acetonitrile/water solution. The solid was dried under reduced pressure to afford additional [difluoro({2-[(4-nitrophenoxy)carbonyl]-1-benzothiophen-5-yl})methyl]phosphonic acid (0.4 g) as a beige solid. Both products were lyophilized to give (difluoro(2-((4-nitrophenoxy)carbonyl)benzo[b]thiophen-5-yl)methyl)phosphonic acid (6.90 g, 16.0 mmol, 98.0% yield). ¹H NMR (400 MHz, DMSO-d₆) δ 7.66-7.74 (m, 3H), 8.27 (d, J=8.3 Hz, 1H), 8.30 (s, 1H), 8.36-8.41 (m, 2H), 8.66 (s, 1H).

The following intermediates in Table 2 were prepared using a similar protocol outlined above for synthesis of [difluoro({2-[(4-nitrophenoxy)carbonyl]-1-benzothiophen-5-yl})methyl]phosphonic acid and utilizing the appropriate advanced intermediate(s) as starting material(s).

TABLE 2

| Name | Structure | LC-MS |
|---|---|---|
| (difluoro(7-((4- nitrophenoxy)carbonyl)naphthalen-2- yl)methyl)phosphonic acid | 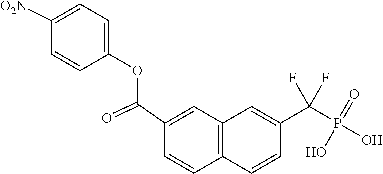 [SMILES: O=C(Oc1ccc([N+](=O)[O-])cc1)c1ccc2ccc(C(F)(F)P(=O)(O)O)cc2c1] | 424.1 [M + H]⁺ |
| (difluoro(2- ((perfluorophenoxy)carbonyl)benzo[b] thiophen-5-yl)methyl)phosphonic acid | 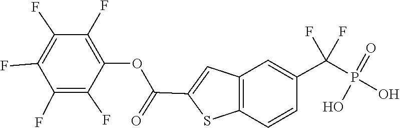 [SMILES: O=C(Oc1c(F)c(F)c(F)c(F)c1F)c1cc2cc(C(F)(F)P(=O)(O)O)ccc2s1] | 475.0 [M + H]⁺ |
| (difluoro(7- ((perfluorophenoxy)carbonyl)naphthalen- 2-yl)methyl)phosphonic acid | 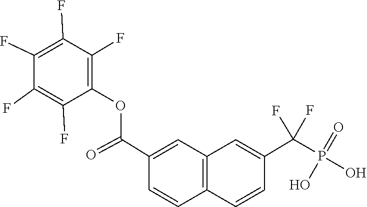 | 467.0 [M − H]⁻ |

### Synthesis of ((2-((perfluorophenoxy)carbonyl)benzo[b]thiophen-5-yl)methyl)phosphonic Acid

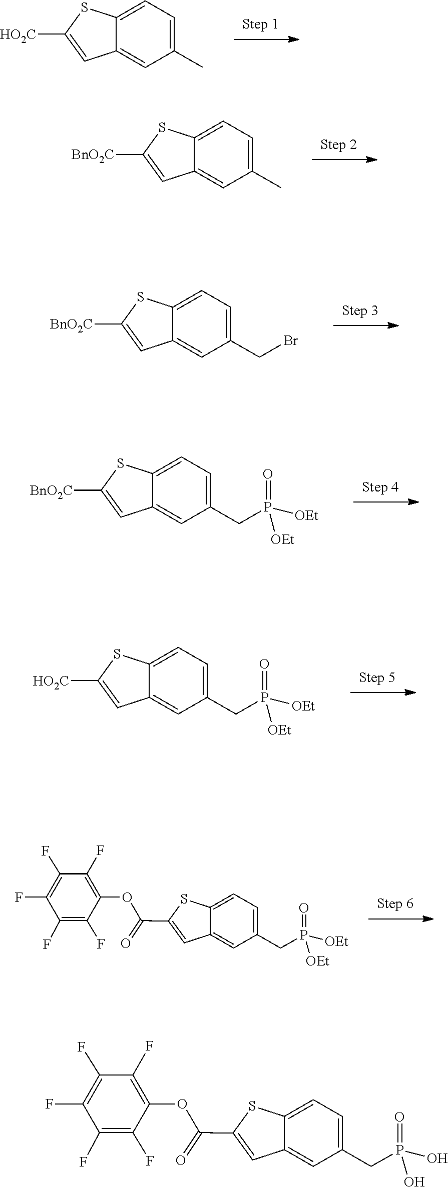

### Preparation of 5-methylbenzo[b]thiophene-2-carboxylic Acid

5-Methylbenzo[b]thiophene-2-carboxylic acid was prepared according to the procedure described in WO 2016/100184 A1.

### Step 1: Preparation of benzyl 5-methylbenzo[b]thiophene-2-carboxylate

To a solution of 5-methylbenzo[b]thiophene-2-carboxylic acid (21.2 g, 110.0 mmol, 1.0 eq) and K₂CO₃ (30.4 g, 220.0 mmol, 2.0 eq) in DMF (200 mL) was added benzyl bromide (20.6 g, 121.0 mmol, 1.1 eq). The mixture was stirred at room temperature for 14 h. The reaction mixture was poured into ice water (400 mL) and stirred for 5 min. The resulting solids were filtered, and the filter cake was washed with water (50 mL), dried in vacuum to give benzyl 5-methylbenzo[b]thiophene-2-carboxylate (30.1 g, 106.0 mmol, 97% yield) as a yellow solid. ¹H NMR (400 MHz, CDCl₃) δ 8.01 (s, 1H), 7.71 (t, J=12.2 Hz, 1H), 7.65 (s, 1H), 7.46 (d, J=6.8 Hz, 2H), 7.42-7.35 (m, 3H), 7.29-7.26 (m, 1H), 5.38 (s, 2H), 2.47 (s, 3H).

### Step 2: Preparation of Benzyl 5-(bromomethyl)benzo[b]thiophene-2-carboxylate

To a solution of benzyl 5-methylbenzo[b]thiophene-2-carboxylate (15.0 g, 53.1 mmol, 1.0 eq) and NBS (10.3 g, 58.4 mmol, 1.1 eq) in CCl₄ (30 mL) was added benzoyl peroxide (1.3 g, 5.31 mmol, 0.1 eq). The reaction flask was subjected to three cycles of evacuation and backfilling with N₂ (g). The mixture was stirred at 80° C. for 16 h under constant atmosphere of N₂ (g). The reaction mixture was concentrated under reduced pressure. The residue was purified by flash column chromatography on silica gel to give benzyl 5-(bromomethyl)benzo[b]thiophene-2-carboxylate (6.80 g, 18.8 mmol, 36% yield) as a yellow solid.

### Step 3: Preparation of Benzyl 5-((diethoxyphosphoryl)methyl)benzo[b]thiophene-2-carboxylate

A solution of benzyl 5-(bromomethyl)benzo[b]thiophene-2-carboxylate (10.3 g, 28.5 mmol, 1.0 eq) dissolved in triethyl phosphite (30.0 g, 180.0 mmol, 6.3 eq) was stirred at 100° C. for 5 h. The reaction mixture was concentrated under reduced pressure directly, the residue was purified by flash column chromatography on silica gel to give benzyl 5-((diethoxyphosphoryl)methyl)benzo[b]thiophene-2-carboxylate (6.5 g, 15.5 mmol, 55% yield) as a colorless oil. LCMS (ESI) m/z=419 [M+H]⁺; ¹H NMR (400 MHz, CDCl₃) δ 8.04 (s, 1H), 7.80 (d, J=8.3 Hz, 2H), 7.49-7.33 (m, 6H), 5.39 (s, 2H), 4.08-3.93 (m, 4H), 3.26 (d, J=21.5 Hz, 2H), 1.24 (t, J=7.1 Hz, 6H).

### Step 4: Preparation of 5-((diethoxyphosphoryl)methyl)benzo[b]thiophene-2-carboxylic Acid

To a solution of benzyl 5-((diethoxyphosphoryl)methyl)benzo[b]thiophene-2-carboxylate (5.6 g, 13.3 mmol, 1.0 eq) in dissolved in a mixture of THF (80 mL) and H₂O (10 mL) was added LiOH (1.10 g, 26.6 mmol, 2.0 eq). The mixture was stirred at room temperature for 3 h and subsequently acidified with aqueous solution of 1 N HCl (adjusted to pH ˜3-4). The product precipitated out of solution upon acidification. The resulting solids were filtered, the filter cake was washed with water (20 mL×2), and the solids were dried under vacuum to give 5-((diethoxyphosphoryl)methyl)benzo[b]thiophene-2-carboxylic acid (3.9 g, 11.8 mmol, 88.9% yield) as a white solid. LCMS (ESI) m/z=329 [M+H]⁺.

### Step 5: Preparation of Perfluorophenyl 5-((diethoxyphosphoryl)methyl)benzo[b]thiophene-2-carboxylate

To a cooled (0° C.) solution of 5-((diethoxyphosphoryl)methyl)benzo[b]thiophene-2-carboxylic acid (3.9 g, 11.8 mmol, 1.0 eq) in CH₂Cl₂ (50 mL) was added oxalyl chloride (2.2 g, 17.7 mmol, 1.5 eq) followed by addition of two drops of DMF. The mixture was stirred at 0° C. for 30 min, followed by evaporation of the reaction mixture to dryness. The resulting solids were dissolved in CH₂Cl₂ (50 mL), followed by addition of Et₃N (3.6 g, 35.4 mmol, 3.0 eq) and pentafluorophenol (2.6 g, 14.1 mmol, 1.2 eq). The resulting mixture was stirred at room temperature for additional 2 h and subsequently, poured over H₂O (30 mL). The bi-phasic solution was extracted with EtOAc (30 mL×3). The combined organic layers were dried over MgSO₄, filtered and concentrated under reduced pressure, the residue was purified by column chromatography on silica gel to give perfluorophenyl 5-((diethoxyphosphoryl)methyl)benzo[b]thiophene-2-carboxylate (4.7 g, 9.5 mmol, 81% yield) as a white solid. LCMS (ESI) m/z=419 [M+H]⁺; ¹H NMR (400 MHz, CDCl₃) δ 8.29 (s, 1H), 7.88 (d, J=9.1 Hz, 2H), 7.51 (d, J=8.4 Hz, 1H), 4.14-3.94 (m, 4H), 3.29 (d, J=21.5 Hz, 2H), 1.26 (t, J=7.0 Hz, 6H).

### Step 6: Preparation of ((2-((perfluorophenoxy)carbonyl)benzo[b]thiophen-5-yl)methyl)phosphonic Acid

To a solution of perfluorophenyl 5-((diethoxyphosphoryl)methyl)benzo[b]thiophene-2-carboxylate (4.7 g, 9.5 mmol, 1.0 eq) in CH₂Cl₂ (60 mL) was added bromotrimethylsilane (12 mL). The mixture was stirred at room temperature for 14 h and subsequently concentrated under reduced pressure. The residue was purified by C18 column chromatography to give ((2-((perfluorophenoxy)carbonyl)benzo[b]thiophen-5-yl)methyl)phosphonic acid (3.7 g, 8.4 mmol, 89% yield) as a white solid. LCMS (ESI) m/z=439 [M+H]⁺.

### Synthesis of (R)- or (S)-5-((diethoxyphosphoryl)fluoromethyl)benzo[b]thiophene-2-carboxylic Acid and (S)-5-((diethoxyphosphoryl)fluoromethyl)benzo[b]thiophene-2-carboxylic Acid

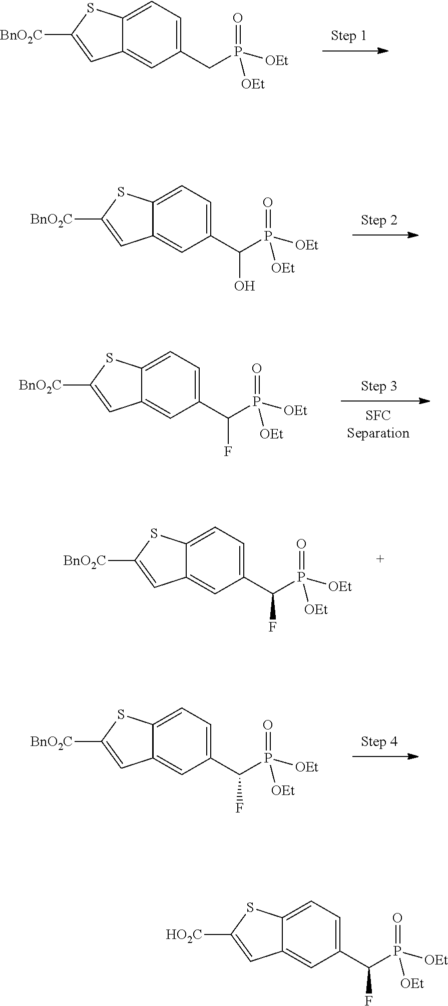

### Step 1: Rac-Benzyl 5-((diethoxyphosphoryl)(hydroxy)methyl)benzo[b]thiophene-2-carboxylate

To a cooled (−78° C.) solution of benzyl 5-((diethoxyphosphoryl)methyl)benzo[b]thiophene-2-carboxylate (2.4 g, 5.73 mmol, 1 eq) in THF (75 mL) and 2-(benzenesulfonyl)-3-phenyloxaziridine (2.97 g, 11.4 mmol, 2 eq) was added a 1 M solution of NaHMDS (11.4 mL, 11.4 mmol, 2 eq) in THF. A deep purple solution was observed upon addition of base that changed to orange after complete addition of the base. The mixture was stirred for an additional 10 min, followed by addition of aqueous saturated NH₄Cl (50 mL). The mixture was warmed to ambient temperatures and EtOAc (75 mL) and water (25 mL) was added. After stirring for an additional 30 min, the phases were separated. The aqueous layer was extracted with EtOAc (125 mL×2). The combined organic extracts were dried with anhydrous sodium sulfate, filtered, concentrated under reduced pressure. Another batch of equal scale was performed and combined for purification. The combined material (6.42 mmol, 12.15 mmol in total) was purified by flash chromatography (20%-100%=EtOAc:heptane) to give rac-benzyl 5-((diethoxyphosphoryl)(hydroxy)methyl)benzo[b]thiophene-2-carboxylate (3.69 g, 8.49 mmol, 70%) as a white sticky solid. LCMS (ESI) m/z=869.4 [2M+H]⁺; ¹H NMR (400 MHz, CDCl₃) δ 8.10 (s, 1H), 8.04-8.00 (m, 1H), 7.88 (d, J=8.6 Hz, 1H), 7.61 (d, J=8.6 Hz, 1H), 7.51-7.47 (m, 2H), 7.46-7.35 (m, 3H), 5.42 (s, 2H), 5.17 (dd, J=10.4, 4.5 Hz, 1H), 4.18-3.95 (m, 4H), 3.10-2.99 (m, 1H), 1.33-1.20 (m, 6H).

### Step 2: Rac-Benzyl 5-((diethoxyphosphoryl)fluoromethyl)benzo[b]thiophene-2-carboxylate

To a cooled (−78° C.) solution (under N₂ (g)) of rac-benzyl 5-((diethoxyphosphoryl)(hydroxy)methyl)benzo[b]thiophene-2-carboxylate (cc) (1.56 g, 3.59 mmol, 1 eq) in CH₂Cl₂ (30 mL) was added (diethylamino)sulfur trifluoride (568 μL, 4.30 mmol, 1.2 eq). The reaction was stirred for 15 min, followed by addition of aqueous saturated NaHCO₃ (50 mL).After warming to room temperature, the product was extracted with CH₂Cl₂ (50 mL×3). The combined organic layers were dried over sodium sulfate, filtered and concentrated under reduced pressure. The crude residue was purified by reverse phase chromatography on C18 cartridge (eluting with 5-80% acetonitrile in water) to give rac-benzyl 5-((diethoxyphosphoryl)fluoromethyl)benzo[b]thiophene-2-carboxylate (650 mg, 1.48 mmol, 41.6%) as a thick clear oil. LCMS (ESI) m/z=873.2 [2M+H]⁺; ¹H NMR (400 MHz, CDCl₃) δ 8.12 (s, 1H), 8.02-7.99 (m, 1H), 7.92 (d, J=8.7 Hz, 1H), 7.61 (d, J=8.7 Hz, 1H), 7.51-7.51 (m, 2H), 7.46-7.36 (m, 3H), 5.82 (dd, J=44.4, 7.5 Hz, 1H), 5.42 (s, 2H), 4.21-4.02 (m, 4H), 1.34-1.26 (m, 6H).

### Step 3: Preparation of Benzyl (R)-5-((diethoxyphosphoryl)fluoromethyl)benzo[b]thiophene-2-carboxylate and benzyl (S)-5-((diethoxyphosphoryl)fluoromethyl)benzo[b]thiophene-2-carboxylate

rac-Benzyl 5-((diethoxyphosphoryl)fluoromethyl)benzo[b]thiophene-2-carboxylate (650 mg, 1.48 mmol) was submitted to chiral SFC separation (Column: Lux i-Amylose 3, 21.2×250 mm 5 um column, 75 mL/min, 40% MeOH) to give benzyl (R)- or (S)-5-((diethoxyphosphoryl)fluoromethyl)benzo[b]thiophene-2-carboxylate (304 mg, 0.70 mmol, 46.8% recovery, 99.9% ee) as a thick clear oil (Peak 1) and benzyl (R)- or (S)-5-((diethoxyphosphoryl)fluoromethyl)benzo[b]thiophene-2-carboxylate (317 mg, 0.73 mmol, 49% recovery, 99.9% ee) as a thick clear oil (Peak 2). Note: Fastest eluting enantiomer by SFC was arbitrarily assigned as (R)-5-(fluoro(phosphono)methyl)benzo[b]thiophene-2-carboxylic acid and slowest eluting enantiomer by SFC as (S)-5-(fluoro(phosphono)methyl)benzo[b]thiophene-2-carboxylic acid.

HPLC method for analysis of enantiomeric excess: Lux Cellulose-3 150 mm 45% H₂O+0.05% TFA/55% MeCN 1 mL/min 8 min.

### Step 4: Preparation of (R)- or (S)-5-((diethoxyphosphoryl)fluoromethyl)benzo[b]thiophene-2-carboxylic Acid

To a mixture of 10% Pd/C (60 mg, 50% wet) and benzyl (R)-5-((diethoxyphosphoryl)fluoromethyl)benzo[b]thiophene-2-carboxylate (Peak 1) (60 mg, 0.1374 mmol, 1 eq) in THF (5 mL) was degassed with N₂ (g) for 5 min. To the mixture was bubbled H₂ (g) for 5 min then the reaction was allowed to stir at room temperature under H₂ (g) (1 atm). The reaction mixture was stirred until consumption of starting material was detected by LCMS. The reaction mixture was subsequently sparged N₂ (g) for 15 min and filtered over a pad of Celite®. The filter cake was washed with 2-MeTHF and the filtrate was concentrated to give (R)- or (S)-5-((diethoxyphosphoryl)fluoromethyl)benzo[b]thiophene-2-carboxylic acid (47.4 mg, 0.0137 mmol, 99%) as a thick clear oil. LCMS (ESI) m/z=347.2 [M+H]⁺.

The following intermediate in Table 3 was prepared using the procedure outlined above (in Step 4) starting from benzyl (S)- or (R)-5-((diethoxyphosphoryl)fluoromethyl)benzo[b]thiophene-2-carboxylate (Peak 2) and using the appropriate reagents.

TABLE 3

| Name | Structure | LCMS |
|---|---|---|
| (S)-or (R)-5- ((diethoxyphosphoryl)fluoromethyl)benzo [b]thiophene-2-carboxylic acid | 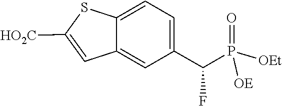 | 347.2 [M + H]⁺ |
| | Or | |
| | 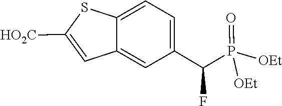 [SMILES: CCOP(=O)(OCC)[C@@H](F)c1ccc2sc(C(=O)O)cc2c1] | |

### Representative Methods for Synthesis of Activated Linkers

### Method 1: Stepwise Acid Chloride Method for Synthesis of Mixed Linkers

### Representative Procedure for the Synthesis of 4-nitrophenyl 5-(((2-(butyrylthio)ethoxy)(pyridin-3-yloxy)phosphoryl)difluoromethyl)benzo[b]thiophene-2-carboxylate

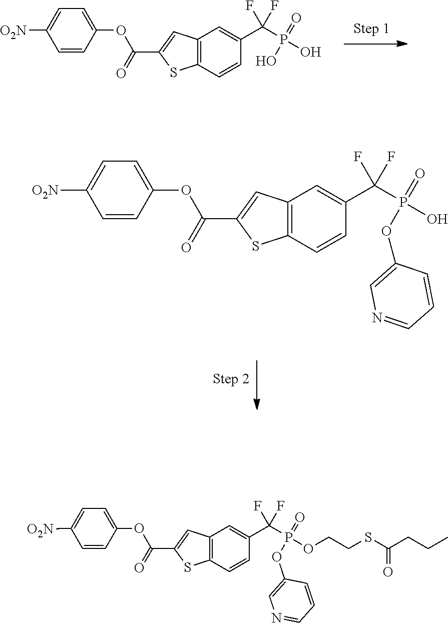

### Step 1: Preparation of 4-nitrophenyl 5-(difluoro(hydroxy(pyridin-3-yloxy)phosphoryl)methyl)benzo[b]thiophene-2-carboxylate

To a cooled (0° C.) heterogeneous solution of (difluoro(2-((4-nitrophenoxy)carbonyl)benzo[b]thiophen-5-yl)methyl)phosphonic acid (100 mg, 0.2329 mmol, 1 eq) in CH₂Cl₂ (4 mL) was added catalytic DMF (2 drops) followed by dropwise addition of oxalyl chloride (198 μL, 2.32 mmol, 10 eq). The homogenous reaction mixture was warmed up to room temperature and stirred for 2 h. The reaction was concentrated in vacuo and further dried under high vacuum for 30 min to give a yellow solid. The yellow solid was diluted in CH₂Cl₂ (4 mL) and cooled down to −78° C. A solution of pyridin-3-ol (22.0 mg, 232 μmol, 1 eq) and triethylamine (64.7 μL, 465 μmol, 2 eq) in CH₂Cl₂ (1 mL) [sonicated for 1 min to allow for solubilization] and was added slowly. The homogeneous reaction mixture was stirred at −78° C. for 2 min, then allowed to warm to ambient temperatures and stirred overnight. After 24 h, the reaction mixture turned heterogeneous and the reaction was concentrated under reduced pressure. The crude product 4-nitrophenyl 5-(difluoro(hydroxy(pyridin-3-yloxy)phosphoryl)methyl)benzo[b]thiophene-2-carboxylate was used directly in the next step without further purification or manipulation. LCMS m/z=507.2 [M+H]⁺.

### Step 2: Preparation of 4-nitrophenyl 5-(((2-(butyrylthio)ethoxy)(pyridin-3-yloxy)phosphoryl)difluoromethyl)benzo[b]thiophene-2-carboxylate

To a cooled (0° C.) solution of nitrophenyl 5-(difluoro(hydroxy(pyridin-3-yloxy)phosphoryl)methyl)benzo[b]thiophene-2-carboxylate (117 mg, 0.2329 mmol, 1 eq) in CH₂Cl₂ (5 mL) was added 2 drops of DMF followed by dropwise addition of oxalyl chloride (198 μL, 2.32 mmol, 10 eq). The reaction was warmed up to room temperature and stirred for 1.5 h. LCMS analysis showed partial conversion to desired activated intermediate. Additional oxalyl chloride (198 μL, 2.32 mmol, 10 eq) was introduced into the reaction mixture and the mixture was stirred for additional 1 h. The reaction was concentrated in vacuo and further dried under high vacuum for 30 min to give a yellow solid. The yellow solid was diluted in CH₂Cl₂ (5 mL) and cooled to −78° C. To the cooled solution was slowly added a solution of 1-[(2-hydroxyethyl)sulfanyl]butan-1-one (103 mg, 698 μmol, 3 eq) diluted in CH₂Cl₂ (1 mL) [previously dried by passing through anhydrous Na₂SO₄] followed by triethylamine (134 μL, 967 μmol, 2 eq). After stirring for 2 min, the resulting mixture was allowed to warm to ambient temperatures and stirred overnight. To the mixture was added Celite® and the mixture was carefully concentrated in vacuo. The crude residue was purified by flash-chromatography (gradient elution 0-60% EtOAc in heptanes) to give 4-nitrophenyl 5-(((2-(butyrylthio)ethoxy)(pyridin-3-yloxy)phosphoryl)difluoromethyl)benzo[b]thiophene-2-carboxylate (22.0 mg, 0.03455 mmol, 14.9% yield) as a clear oil. LCMS m/z=637.2 [M+H]⁺; ¹H NMR: (400 MHz, DMSO-d₆) δ 8.50-8.44 (m, 2H), 8.33-8.38 (m, 3H), 8.25 (s, 1H), 8.04 (d, J=8.0 Hz, 1H), 7.79 (d, J=8.6 Hz, 1H), 7.57-7.53 (m, 1H), 7.50-7.45 (m, 2H), 7.31-7.26 (m, 1H), 4.37-4.24 (m, 2H), 3.20-3.08 (m, 2H), 2.52 (t, J=7.6 Hz, 2H), 1.67 (sextet, J=7.3 Hz, 2H), 0.94 (t, J=7.6 Hz, 3H).

The following intermediates in Table 4 were prepared using a similar protocol described above for synthesis of 4-nitrophenyl 5-(((2-(butyrylthio)ethoxy)(pyridin-3-yloxy)phosphoryl)difluoromethyl)benzo[b]thiophene-2-carboxylate and utilizing the appropriate advanced intermediate(s) as starting material(s).

TABLE 4

| Name | Structure | LCMS | NMR |
|---|---|---|---|
| 4-nitrophenyl 5- (((2- (butyrylthio) ethoxy)(phenoxy) phosphoryl) difluoro methyl)benzo[b] thiophene-2- carboxylate | 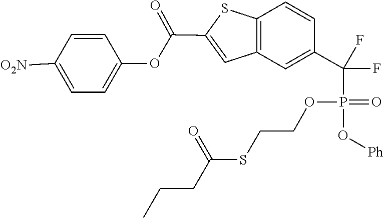 [SMILES: CCCC(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.44 (s, 1H), 8.40-8.35 (m, 3H), 7.79 (d, J = 8.8 Hz, 1H), 7.70 (d, J = 9.0 Hz, 2H), 7.44-7.37 (m, 2H), 7.29-7.23 (m, 1H), 7.18 (d, J = 8.6 Hz, 2H), 4.36- 4.20 (m, 2H), 3.15 (t, J = 6.1Hz, 2H), 2.58-2.52 (m, 2H), 1.58-1.50 (m, 2H), 0.85 (q, J = 7.3 Hz, 3H) |
| 4-nitrophenyl 5- (((2- (butyrylthio) ethoxy) (naphthalen-1- yloxy) phosphoryl) difluoromethyl) benzo[b] thiophene-2- carboxylate | 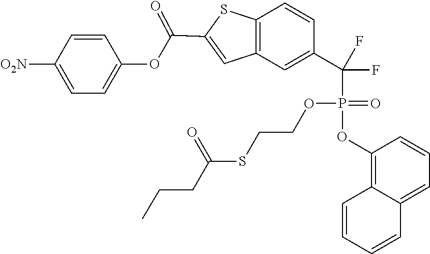 [SMILES: CCCC(=O)SCCOP(=O)(Oc1cccc2ccccc12)C(F)(F)c1ccc2sc(C(=O)Oc3ccc([N+](=O)[O-])cc3)cc2c1] | | ¹H NMR (400 MHz, DMSO-d₆) δ 8.65 (s, 1H), 8.49 (s, 1H), 8.39 (d, J = 9.3 Hz, 3H), 8.00 (d, J = 8.3 Hz, 1H), 7.87-7.80 (m, 3H), 7.70 (d, J = 9.0 Hz, 2H), 7.64-7.58 (m, 1H), 7.58- 7.53 (m, 1H), 7.49 (t, J = 7.9 Hz, 1H), 7.38 (d, J = 7.6 Hz, 1H), 4.35-4.23 (m, 2H), 3.11 (t, J = 5.9 Hz, 2H), 2.45 (t, J = 7.3 Hz, 2H), 1.48 (sextet, J = 7.4 Hz, 2H), 0.81 (t, J = 7.5 Hz, 3H) |
| 4-nitrophenyl 5- (difluoro(phenoxy (2-(pivaloylthio) ethoxy) phosphoryl) methyl)benzo[b] thiophene-2- carboxylate | 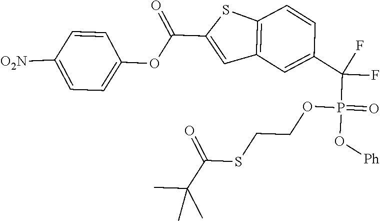 [SMILES: CC(C)(C)C(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.49-8.34 (m, 4H), 7.80 (d, J = 8.8 Hz, 1H), 7.70 (d, J = 9.0 Hz, 2H), 7.41 (t, J = 7.6 Hz, 2H), 7.30-7.15 (m, 3H), 4.33-4.23 (m, 2H), 3.12 (t, J = 5.6 Hz, 2H), 1.12 (s, 9H) |
| 4-nitrophenyl 5- (((4- cyanophenoxy)(2- (pivaloylthio) ethoxy) phosphoryl) difluoromethyl) benzo[b] thiophene-2- carboxylate | 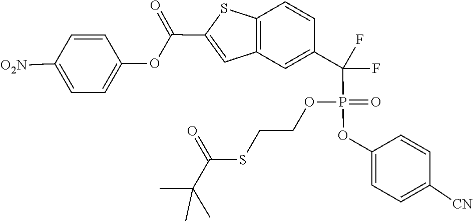 [SMILES: CC(C)(C)C(=O)SCCOP(=O)(Oc1ccc(C#N)cc1)C(F)(F)c1ccc2sc(C(=O)Oc3ccc([N+](=O)[O-])cc3)cc2c1] | | |
| 4-nitrophenyl 5- (((3- cyanophenoxy)(2- (pivaloylthio) ethoxy) phosphoryl) difluoromethyl) benzo[b] thiophene-2- carboxylate | 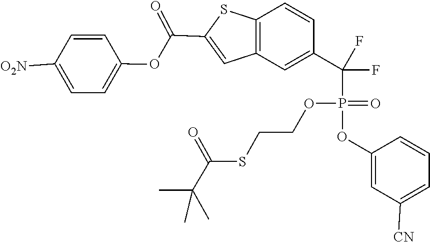 [SMILES: CC(C)(C)C(=O)SCCOP(=O)(Oc1cccc(C#N)c1)C(F)(F)c1ccc2sc(C(=O)Oc3ccc([N+](=O)[O-])cc3)cc2c1] | | ¹H NMR (400 MHz, DMSO-d₆) δ 8.66 (s, 1H), 8.46 (s, 1H), 8.42-8.34 (m, 3H), 7.83-7.67 (m, 5H), 7.66-7.59 (m, 1H), 7.58- 7.53 (m, 1H), 4.40-4.28 (m, 2H), 3.14 (t, J = 6.2 Hz, 2H), 1.12 (s, 9H) |
| 4-nitrophenyl 5- (((2- (butyrylthio) ethoxy)(3- cyanophenoxy) phosphoryl) difluoro methyl)benzo[b] thiophene-2- carboxylate | 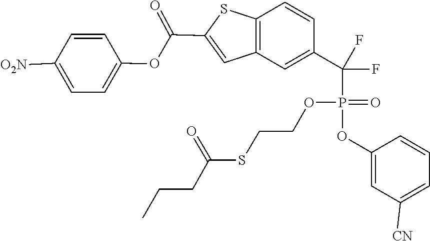 [SMILES: CCCC(=O)SCCOP(=O)(Oc1cccc(C#N)c1)C(F)(F)c1ccc2sc(C(=O)Oc3ccc([N+](=O)[O-])cc3)cc2c1] | 661.1 [M + H]⁺ | |
| 4-nitrophenyl 5- (difluoro((2-((3- methylbutanoyl) thio)ethoxy) (phenoxy) phosphoryl) methyl)benzo[b] thiophene-2- carboxylate | 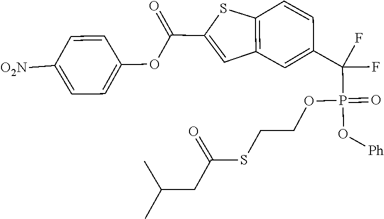 [SMILES: CC(C)CC(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.49-8.34 (m, 4H), 7.80 (d, J = 8.8 Hz, 1H), 7.70 (d, J = 9.0 Hz, 2H), 7.41 (t, J = 7.6 Hz, 2H), 7.30-7.15 (m, 3H), 4.33-4.23 (m, 2H), 3.12 (t, J = 5.6 Hz, 2H), 1.12 (s, 9H) |
| perfluorophenyl 5-(((2-((3-((tert- butyldimethylsilyl) oxy)-2,2- dimethylpropanoyl) thio)ethoxy) (phenoxy) phosphoryl) difluoromethyl) benzo[b]thiophene- 2-carboxylate | 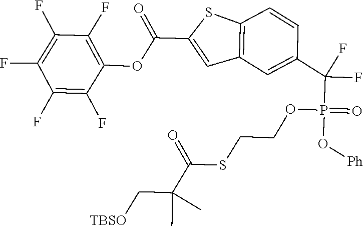 [SMILES: CC(C)(CO[Si](C)(C)C(C)(C)C)C(=O)SCCOP(=O)(Oc1ccccc1)C(F)(F)c1ccc2sc(C(=O)Oc3c(F)c(F)c(F)c(F)c3F)cc2c1] | 847.0 [M + Na]⁺ | |

### Method 2: One-Pot Acid Chloride Method for the Synthesis of Activated Linkers

### Representative Procedure for the Synthesis of Perfluorophenyl 5-((bis(4-((3-methylbutanoyl)thio)butoxy)phosphoryl)difluoromethyl)benzo[b]thiophene-2-carboxylate

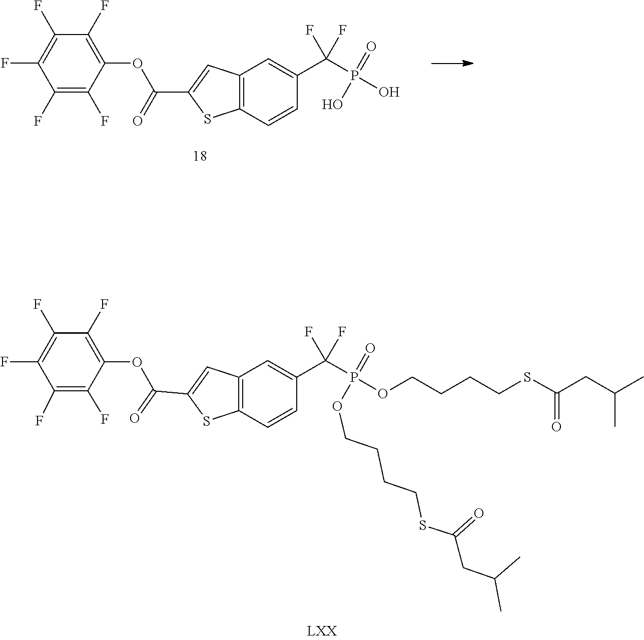 [SMILES: O=C(Oc1c(F)c(F)c(F)c(F)c1F)c1cc2cc(C(F)(F)P(=O)(O)O)ccc2s1 ; CC(C)CC(=O)SCCCCOP(=O)(OCCCCSC(=O)CC(C)C)C(F)(F)c1ccc2sc(C(=O)Oc3c(F)c(F)c(F)c(F)c3F)cc2c1]

To a cooled (0° C.) solution of 3 (200 mg, 0.42 mmol, 1.0 eq) in dry CH₂Cl₂ (15 mL) and catalytic DMF (3.2 μL, 42.1 μmol, 0.1 eq) was added in a dropwise manner oxalyl chloride (266 mg, 2.10 mmol, 5.0 eq). The reaction mixture was allowed to warm to 40° C. After stirring for 2 h, the reaction mixture was concentrated in vacuo and dried (to remove excess oxalyl chloride). The resulting solids were re-dissolved in anhydrous CH₂Cl₂ (5 mL) and cooled to 0° C. To the cooled solution was added S-(4-hydroxybutyl) 3-methylbutanethioate (239 mg, 1.26 mmol, 3.0 eq), DMAP (5.14 mg, 42.1 μmol, 0.1 eq) and a solution of N,N-diisopropylethylamine (217 mg, 1.68 mmol, 4.0 eq) in anhydrous CH₂Cl₂ (10 mL). The reaction mixture was allowed to warm to room temperature and stirred for additional 18 h. The reaction was quenched by adding H₂O (10 mL) and extracted with CH₂Cl₂ (3×10 mL). The organic layers were combined and washed with brine (20 mL), dried over anhydrous Na₂SO₄, and concentrated in vacuo. The residue was purified by flash column chromatography to afford perfluorophenyl 5-((bis(4-((3-methylbutanoyl)thio)butoxy)phosphoryl)difluoromethyl)benzo [b]thiophene-2-carboxylate (15.0 mg, 18.3 μmol, 4.4% yield). LCMS (ESI) m/z=819 [M+H]⁺.

The following intermediates in Table 5 were prepared using a similar protocol described above for synthesis of perfluorophenyl 5-((bis(4-((3-methylbutanoyl)thio)butoxy)phosphoryl)difluoromethyl)benzo [b]thiophene-2-carboxylate and utilizing the appropriate advanced intermediate(s) as starting material(s).

TABLE 5

| Name | Structure | LC-MS | NMR |
|---|---|---|---|
| 4-nitrophenyl 5- ((bis(2- (pivaloylthio)ethoxy) phosphoryl)difluoro methyl)benzo[b] thiophene-2- carboxylate | 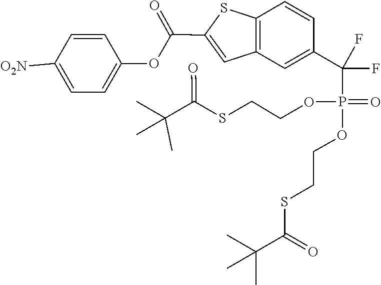 [SMILES: CC(C)(C)C(=O)SCCOP(=O)(OCCSC(=O)C(C)(C)C)C(F)(F)c1ccc2sc(C(=O)Oc3ccc([N+](=O)[O-])cc3)cc2c1] | | ¹H NMR (400 MHz, DMSO-d₆) δ 8.66 (s, 1H), 8.41-8.32 (m, 4H), 7.76- 7.65 (m, 3H), 4.22- 4.11 (m, 4H), 3.12 (t, J = 6.4 Hz, 4H), 1.16 (s, 18H) |
| perfluorophenyl 5- ((bis(2- (pivaloylthio)ethoxy) phosphoryl)methyl) benzo[b]thiophene- 2-carboxylate | 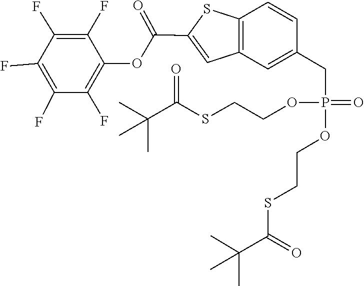 [SMILES: CC(C)(C)C(=O)SCCOP(=O)(Cc1ccc2sc(C(=O)Oc3c(F)c(F)c(F)c(F)c3F)cc2c1)OCCSC(=O)C(C)(C)C] | 749.0 [M + Na]⁺ | |

### Method 3: One-Pot Silver Salt Method for the Synthesis of Activated Linkers

### Representative Procedure for the Synthesis of Perfluorophenyl 5-((bis(((isopropoxycarbonyl)oxy)methoxy)phosphoryl)methyl)benzo[b]thiophene-2-carboxylate

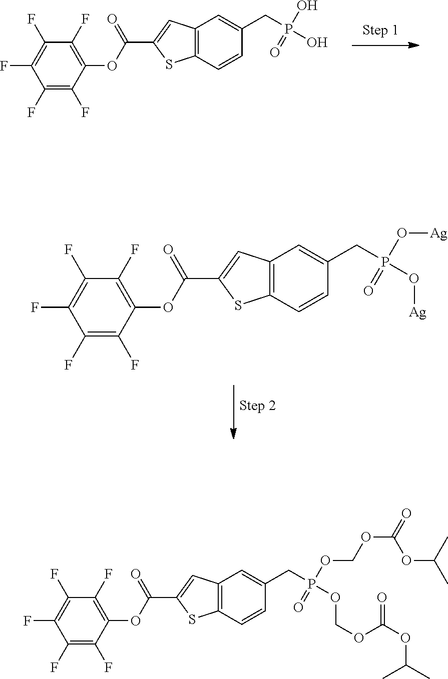

### Step 1: Preparation of silver(I) ((2-((perfluorophenoxy)carbonyl)benzo[b]thiophen-5-yl)methyl)phosphonate

To a solution of ((2-((perfluorophenoxy)carbonyl)benzo[b]thiophen-5-yl)methyl)phosphonic acid (300 mg, 684 μmol, 1.0 eq) in a mixture of deionized H₂O (4 mL) and THF (2 mL) was added Amberlite IR120® resin (Na⁺ form) (1.5 g). The resulting mixture was stirred at room temperature for 1 h and the suspension was subsequently filtered. To the filtrate was added a solution of AgNO₃ (463 mg, 2.73 mmol, 4.0 eq) in deionized H₂O (2 mL) and the resulting mixture was stirred at room temperature for an additional 1 h. Formation of a white precipitate was observed and the solids were collected via filtration. The filter cake was then washed with cold H₂O (3×2 mL), and the solid was dried under reduced pressure to yield silver(I) ((2-((perfluorophenoxy)carbonyl)benzo[b]thiophen-5-yl)methyl)phosphonate as a dry powder. The silver salt was used without further purification.

### Step 2: Preparation of Perfluorophenyl 5-((bis(((isopropoxycarbonyl)oxy)methoxy)phosphoryl)methyl)benzo[b]thiophene-2-carboxylate

To a suspension of silver(I) ((2-((perfluorophenoxy)carbonyl)benzo[b]thiophen-5-yl)methyl)phosphonate was suspended in anhydrous toluene (10 mL) and iodomethyl 2-methylpropanoate (500 mg, 2.05 mmol, 3.0 eq) was added in a dropwise manner. After addition, the resulting mixture was stirred at room temperature for an additional 12 h. The reaction progress was monitored by LCMS, and after completion, the unreacted silver salt was recovered by filtration. The filtrate solution was concentrated in vacuo, and the resulting residue was purified by reverse phase chromatography [C18 column gradient elution water/acetonitrile=90% to 1%] to give perfluorophenyl 5-((bis(((isopropoxycarbonyl)oxy)methoxy)phosphoryl)methyl)benzo[b]thiophene-2-carboxylate (165 mg, 246 μmol, 36% yield) as a white solid. LCMS (ESI) m/z=671 [M+H]⁺; ¹H NMR (400 MHz, CDCl₃) δ 8.28 (s, 1H), 7.96-7.80 (m, 2H), 7.48 (d, J=8.5 Hz, 1H), 5.70-5.53 (m, 4H), 4.90 (dt, J=12.6, 6.2 Hz, 2H), 3.42 (d, J=22.2 Hz, 2H), 1.31 (d, J=6.2 Hz, 12H).

The following intermediates in Table 6 were prepared using a similar protocol described above for synthesis of perfluorophenyl 5-((bis(4-((3-methylbutanoyl)thio)butoxy)phosphoryl)difluoromethyl)benzo[b]thiophene-2-carboxylate and utilizing the appropriate advanced intermediate(s) as starting material(s).

TABLE 6

| Name | Structure | LCMS | NMR |
|---|---|---|---|
| perfluorophenyl 5- ((bis(2- (butyrylthio)ethoxy) phosphoryl)methyl) benzo[b]thiophene- 2-carboxylate | 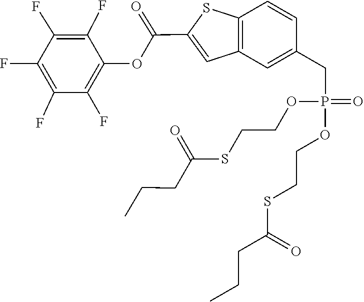 [SMILES: CCCC(=O)SCCOP(=O)(Cc1ccc2sc(C(=O)Oc3c(F)c(F)c(F)c(F)c3F)cc2c1)OCCSC(=O)CCC] | 699 [M + H]⁺ | ¹H NMR (400 MHZ, CDCl₃) δ 8.31 (s, 1H), 7.96-7.80 (m, 2H), 7.50 (d, J = 8.3 Hz, 1H), 4.08- 4.03 (m, 4H), 3.33 (d, J_{HP} = 21.6 Hz, 2H), 3.08 (t, J = 6.5 Hz, 4H), 2.52 (t, J = 7.4 Hz, 4H), 1.72- 1.63 (m, 4H), 0.94 (t, J = 7.4 Hz, 6H) |
| 4-nitrophenyl 5- ((bis(2- (butyrylthio)ethoxy) phosphoryl) difluoromethyl) benzo[b]thiophene- 2-carboxylate | 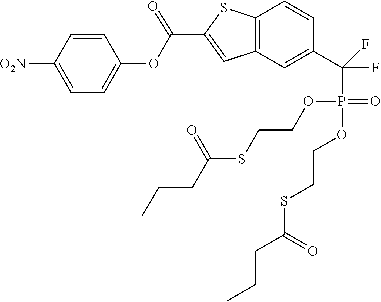 [SMILES: CCCC(=O)SCCOP(=O)(OCCSC(=O)CCC)C(F)(F)c1ccc2sc(C(=O)Oc3ccc([N+](=O)[O-])cc3)cc2c1] | 690 [M + H]⁺ | ¹H NMR (400 MHZ, CDCl₃) δ 8.35-8.34 (m, 3H), 8.23 (s, 1H), 8.03 (d, J = 8.4 Hz, 1H), 7.76 (d, J = 8.7 Hz, 1H), 7.48 (d, J = 9.0 Hz, 2H), 4.29-4.19 (m, 4H), 3.27-3.03 (m, 4H), 2.54 (t, J = 7.4 Hz, 4H), 1.75-1.63 (m, 4H), 0.95 (t, J = 7.4 Hz, 6H) |
| perfluorophenyl 5- ((bis(2-((3- methylbutanoyl) thio)ethoxy) phosphoryl) difluoromethyl) benzo[b]thiophene- 2-carboxylate | 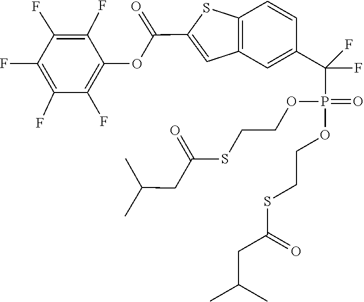 [SMILES: CC(C)CC(=O)SCCOP(=O)(OCCSC(=O)CC(C)C)C(F)(F)c1ccc2sc(C(=O)Oc3c(F)c(F)c(F)c(F)c3F)cc2c1] | | ¹H NMR (400 MHZ, CDCl₃) δ 8.41 (s, 1H), 8.24 (s, 1H), 8.03 (d, J = 8.6 Hz, 1H), 7.78 (d, J = 8.7 Hz, 1H), 4.34-4.08 (m, 4H), 3.25-3.05 (m, 4H), 2.43 (d, J = 7.1 Hz, 4H), 2.14 (dt, J = 13.7, 6.8 Hz, 2H), 0.95 (s, 6H), 0.94 (s, 6H) |
| ((((2- ((perfluorophenoxy) carbonyl)benzo[b] thiophen-5- yl)methyl) phosphoryl)bis(oxy)) bis(methylene)bis(2, 2-dimethylpropanoate | 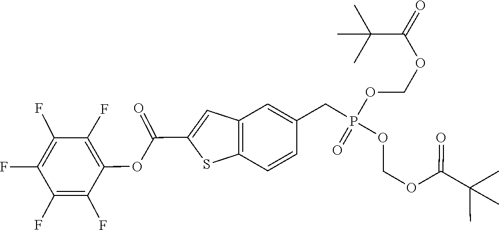 [SMILES: CC(C)(C)C(=O)OCOP(=O)(Cc1ccc2sc(C(=O)Oc3c(F)c(F)c(F)c(F)c3F)cc2c1)OCOC(=O)C(C)(C)C] | 689 [M + Na]⁺ | ¹H NMR (400 MHZ, CDCl₃) δ 8.28 (s, 1H), 7.90-7.86 (m, 2H), 7.50- 7.45 (m, 1H), 5.68-5.56 (m, 4H), 3.38 (m, 2H), 1.19 (s, 18H) |
| (((difluoro(7-((4- nitrophenoxy) carbonyl)naphthalen- 2-yl)methyl) phosphoryl)bis(oxy)) bis(methylene)bis(2, 2-dimethylpropanoate) | 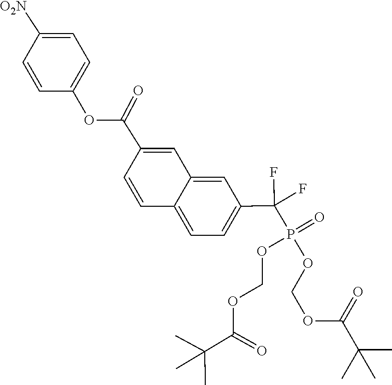 [SMILES: CC(C)(C)C(=O)OCOP(=O)(OCOC(=O)C(C)(C)C)C(F)(F)c1ccc2ccc(C(=O)Oc3ccc([N+](=O)[O-])cc3)cc2c1] | 674.1 [M + Na]⁺ | ¹H NMR (400 MHZ, CDCl₃) δ 8.90 (s, 1H), 8.40-8.34 (m, 2H), 8.34- 8.26 (m, 2H), 8.04 (d, J = 8.4 Hz, 2H), 7.85 (d, J = 8.4 Hz, 1H), 7.52-7.47 (m, 2H), 5.81-5.66 (m, 4H), 1.21 (s, 18H) |
| perfluorophenyl 5- ((bis(2-((3-((tert- butyldimethylsilyl) oxy)-3- methylbutanoyl) thio)ethoxy) phosphoryl) difluoromethyl)benzo [b]thiophene- 2-carboxylate | 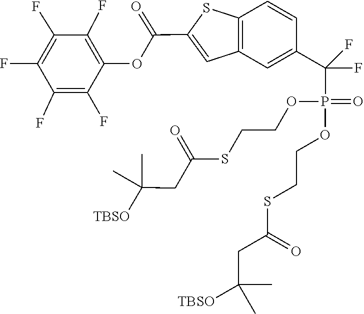 [SMILES: CC(C)(CC(=O)SCCOP(=O)(OCCSC(=O)CC(C)(C)O[Si](C)(C)C(C)(C)C)C(F)(F)c1ccc2sc(C(=O)Oc3c(F)c(F)c(F)c(F)c3F)cc2c1)O[Si](C)(C)C(C)(C)C] | | ¹H NMR (400 MHZ, CDCl₃) δ 8.39-8.30 (m, 3H), 8.22 (s, 1H), 8.01 (d, J = 8.6 Hz, 1H), 7.75 (d, J = 8.5 Hz, 1H), 7.47 (d, J = 8.9 Hz, 2H), 4.25-4.13 (m, 4H), 3.57 (s, 4H), 3.16- 3.05 (m, 4H), 1.18 (s, 12H), 0.84 (s, 18H), 0.00 (s, 12H) |
| perfluorophenyl 5- ((bis(2-((3,3,3- trifluoro-2,2- dimethylpropanoyl) thio)ethoxy) phosphoryl) difluoromethyl)benzo [b]thiophene-2- carboxylate | 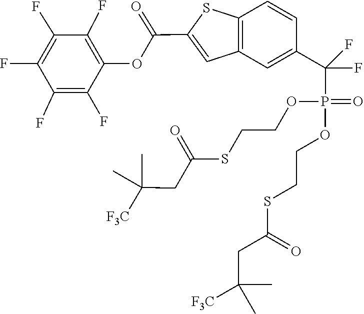 [SMILES: CC(C)(CC(=O)SCCOP(=O)(OCCSC(=O)CC(C)(C)C(F)(F)F)C(F)(F)c1ccc2sc(C(=O)Oc3c(F)c(F)c(F)c(F)c3F)cc2c1)C(F)(F)F] | 893.2 [M + Na]⁺ | |
| perfluorophenyl 7- ((bis(2-((3- methylbutanoyl) thio)ethoxy)phosphor yl)difluoromethyl)- 2-naphthoate | 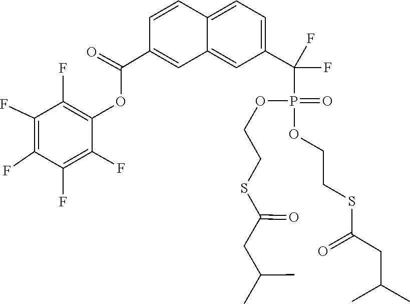 [SMILES: CC(C)CC(=O)SCCOP(=O)(OCCSC(=O)CC(C)C)C(F)(F)c1ccc2ccc(C(=O)Oc3c(F)c(F)c(F)c(F)c3F)cc2c1] | 799 [M + Na]⁺ | |
| perfluorophenyl 5- ((bis (((isopropoxycarbonyl) oxy)methoxy) phosphoryl)methyl) benzo[b]thiophene-2- carboxylate | 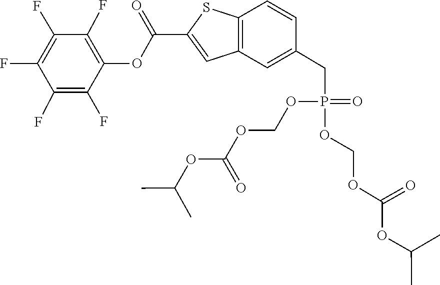 [SMILES: CC(C)OC(=O)OCOP(=O)(Cc1ccc2sc(C(=O)Oc3c(F)c(F)c(F)c(F)c3F)cc2c1)OCOC(=O)OC(C)C] | | ¹H NMR (400 MHz, CDCl₃) δ 8.28 (s, 1H), 7.96-7.80 (m, 2H), 7.48 (d, J = 8.5 Hz, 1H), 5.70- 5.53 (m, 4H), 4.90 (dt, J = 12.6, 6.2 Hz, 2H), 3.42 (d, J = 22.2 Hz, 2H), 1.31 (d, J = 6.2 Hz, 12H) |
| perfluorophenyl 5- ((bis(2-((3-((tert- butyldimethylsilyl) oxy)-3- methylbutanoyl) thio)ethoxy) phosphoryl)methyl) benzo[b]thiophene-2- carboxylate | 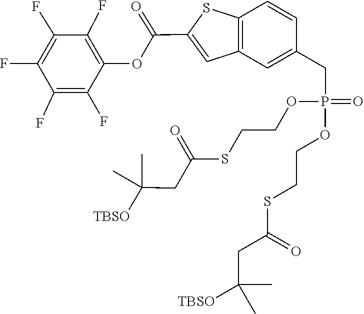 [SMILES: CC(C)(CC(=O)SCCOP(=O)(Cc1ccc2sc(C(=O)Oc3c(F)c(F)c(F)c(F)c3F)cc2c1)OCCSC(=O)CC(C)(C)O[Si](C)(C)C(C)(C)C)O[Si](C)(C)C(C)(C)C] | | ¹H NMR (400 MHZ, CDCl₃) 8 8.30 (s, 1H), 7.95-7.80 (m, 2H), 7.50 (d, J = 8.4 Hz, 1H), 4.01 (dd, J = 14.7, 7.0 Hz, 4H), 3.58 (d, J= 10.6 Hz, 4H), 3.32 (d, J = 21.6 Hz, 2H), 3.04 (t, J = 6.7 Hz, 4H), 1.18 (d, J = 8.5 Hz, 12H), 0.84 (s, 18H), 0.00 (d, J = 3.5 Hz, 12H) |

### (R)- or (S)-5-((bis((pivaloyloxy)methoxy)phosphoryl)fluoromethyl)benzo[b]thiophene-2-carboxylic Acid

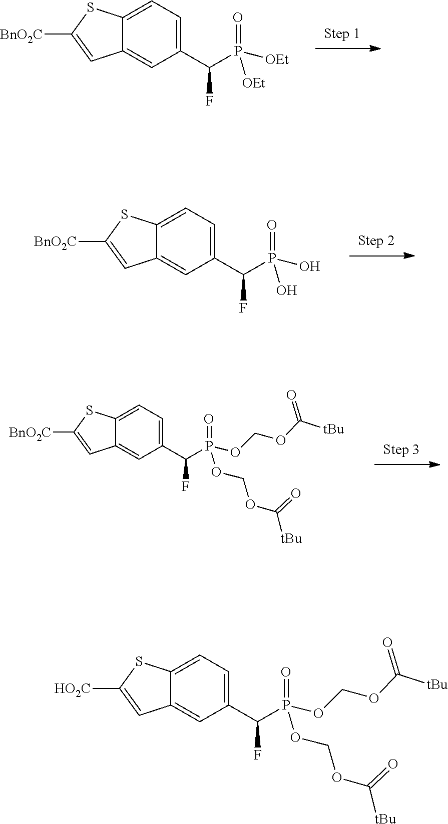

### Step 1: Preparation of (R)- or (S)-((2-((benzyloxy)carbonyl)benzo[b]thiophen-5-yl)fluoromethyl)phosphonic Acid

To a cooled (0° C.) solution of benzyl (R)- or (S)-5-((diethoxyphosphoryl)fluoromethyl)benzo[b]thiophene-2-carboxylate (Peak 1) (205 mg, 0.4697 mmol) in CH₂Cl₂ (8.0 mL) was added BSTFA (746 μL, 2.81 mmol) followed by a 1.0 M solution of trimethylsilyl iodide (1.87 mL, 1.87 mmol) in CH₂Cl₂. After stirring for 1 h, a mixture of acetonitrile (0.66 mL), water (0.33 mL) and 0.1% TFA was added. The solvent was removed under reduced pressure at 0° C. The crude residue was purified by reverse phase chromatography (C18 cartridge eluting with 5-40% acetonitrile in water) to give (R)- or (S)-((2-((benzyloxy)carbonyl)benzo[b]thiophen-5-yl)fluoromethyl)phosphonic acid (163 mg, 0.4285 mmol) as a white solid. ¹H NMR (400 MHz, DMSO-d₆) δ 8.33 (s, 1H), 8.11-8.06 (m, 2H), 7.58 (d, J=8.6 Hz, 1H), 7.51-7.47 (m, 2H), 7.45-7.33 (m, 3H), 5.84 (dd, J=44.6, 8.3 Hz, 1H), 5.40 (s, 2H).

### Step 2: Preparation of (R)- or (S)-((2-((benzyloxy)carbonyl)benzo[b]thiophen-5-yl)fluoromethyl)phosphonic Acid

[The following reaction was conducted in foiled covered vessel and in the absence of ambient light]: To a suspension of (R)- or (S)-((2-((benzyloxy)carbonyl)benzo[b]thiophen-5-yl)fluoromethyl)phosphonic acid (163 mg, 0.4285 mmol, 1 eq) in water (5 mL) was added a solution of aqueous sodium hydroxide (34.2 mg, 857 μmol, 2 eq) in water (2 mL). To the yellow solution was added silver(I) nitrate (181 mg, 1.07 mmol, 2.5 eq) and the resulting off-white suspension was stirred for 1.5 h at room temperature. The suspension was cooled to 0° C., filtered, and dried under high vacuum. The solids were re-suspended in acetonitrile, concentrated under reduced pressure, and further dried under high vacuum (3 h). The resulting dark yellow powder was suspended in toluene (10 mL) and iodomethyl 2,2-dimethylpropanoate (191 μL, 1.28 mmol, 3 eq) was added. After stirring for 20 h, the reaction mixture was stirred for 20 h at room temperature. The reaction mixture was filtered and rinsed with toluene. The filtrate was concentrated under reduced pressure. The crude residue was purified (C18 cartridge eluting with 5-100% acetonitrile in water) to give (R)- or (S)-((((2-((benzyloxy)carbonyl)benzo[b]thiophen-5-yl)fluoromethyl)phosphoryl)bis(oxy))bis(methylene) bis(2,2-dimethylpropanoate) (122 mg, 0.2004 mmol, 46.9%) as a clear oil. ¹H NMR (400 MHz, CDCl₃) δ 8.11 (s, 1H), 8.01-7.99 (m, 1H), 7.92 (d, J=8.3 Hz, 1H), 7.59 (d, J=8.3 Hz, 1H), 7.52-7.47 (m, 2H), 7.46-7.36 (m, 3H), 8.87 (dd, J=44.3, 7.5 Hz, 1H), 5.72-5.62 (m, 4H), 5.43 (s, 2H), 1.21 (s, 18H).

### Step 3: Preparation of (R)- or (S)-5-((bis((pivaloyloxy)methoxy)phosphoryl)fluoromethyl)benzo[b]thiophene-2-carboxylic Acid

To a solution of (R)-((((2-((benzyloxy)carbonyl)benzo[b]thiophen-5-yl)fluoromethyl)phosphoryl)bis(oxy))bis(methylene) bis(2,2-dimethylpropanoate) (122 mg, 0.200 mmol, 1 eq) in THF (10 mL) under N₂ (g) was added 10% Pd/C (50% wet, 120 mg, 0.1127 mmol, 0.56 eq). To the suspension was bubbled H₂ (g) for 5 min. The reaction mixture was stirred under H₂ (g) (1 atm) at room temperature. After stirring for 22 h, the reaction mixture was purged with N₂ (g) and filtered over Celite®. The filter pad was washed with THF and concentrated under reduced pressure to give (R)-5-((bis((pivaloyloxy)methoxy)phosphoryl)fluoromethyl)benzo[b]thiophene-2-carboxylic acid (0.200 mmol, 99.9%) as a thick clear oil. LC-MS (ESI) m/z [M+H]⁺=519.1.

The following intermediates in Table 7 were prepared using a similar protocol described above for synthesis of (R)- or (S)-5-((bis((pivaloyloxy)methoxy)phosphoryl)fluoromethyl)benzo[b]thiophene-2-carboxylic acid and utilizing the appropriate advanced intermediate(s) as starting material(s). The absolute configuration of the starting material, (R)- or (S)-((2-((benzyloxy)carbonyl)benzo[b]thiophen-5-yl)fluoromethyl)phosphonic acid was not determined, but the elution peak (“Peak 1” or “Peak 2”) of the starting material utilized is indicated in the table

TABLE 7

| Name | Structure | LCMS | NMR |
|---|---|---|---|
| (R)-or (S)-5- ((bis((pivaloyloxy) methoxy)phosphoryl) fluoromethyl)benzo[b] thiophene-2-carboxylic acid | 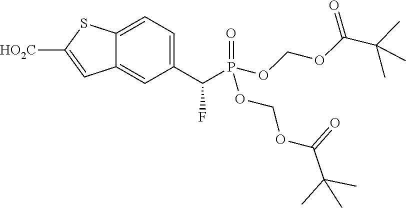 [SMILES: CC(C)(C)C(=O)OCOP(=O)(OCOC(=O)C(C)(C)C)[C@H](F)c1ccc2sc(C(=O)O)cc2c1] Or 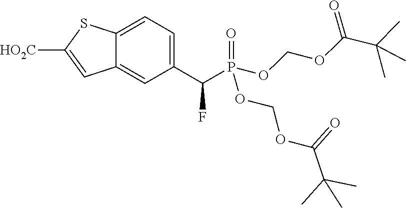 [SMILES: CC(C)(C)C(=O)OCOP(=O)(OCOC(=O)C(C)(C)C)[C@@H](F)c1ccc2sc(C(=O)O)cc2c1] (Peak 2 of phosphonate building block was utilized for synthesis) | 519.1 [M + H]⁺ | |
| (R)-or (S)-5- ((bis (((isopropoxycarbonyl) oxy)methoxy) phosphoryl) fluoromethyl)benzo[b] thiophene-2-carboxylic acid | 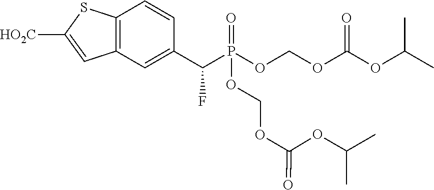 [SMILES: CC(C)OC(=O)OCOP(=O)(OCOC(=O)OC(C)C)[C@H](F)c1ccc2sc(C(=O)O)cc2c1] Or 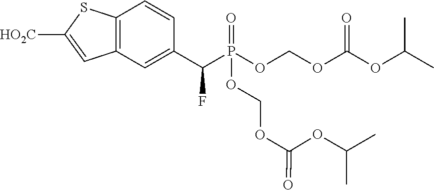 [SMILES: CC(C)OC(=O)OCOP(=O)(OCOC(=O)OC(C)C)[C@@H](F)c1ccc2sc(C(=O)O)cc2c1] (Peak 2 of phosphonate building block was utilized for synthesis) | | ¹H NMR (400 MHZ, CDCl₃) δ 8.35 (s, 1H), 8.09 (s, 1H), 7.97 (d, J = 8.5 Hz, 1H), 7.65 (d, J = 8.6 Hz, 1H), 5.93 (dd, J = 44.2, 7.3 Hz, 1H), 5.73- 5.54 (m, 4H), 4.97- 4.84 (m, 2H), 1.33- 1.29 (m, 12H) |

### Method 4: Stepwise Silver Salt Method for the Synthesis of Mixed Linkers

### Representative Procedure for the Perfluorophenyl 5-(((2-(butyrylthio)ethoxy)(2-(pivaloylthio)ethoxy)phosphoryl)difluoromethyl)benzo[b]thiophene-2-carboxylate

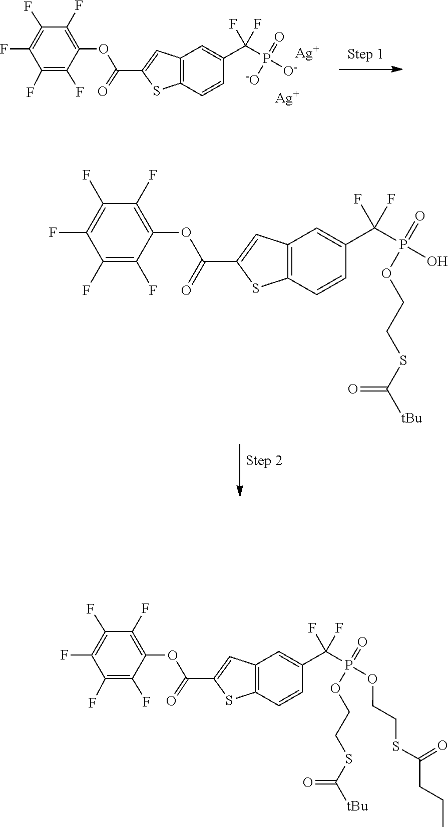

### Step 1: Preparation of Perfluorophenyl 5-(difluoro(hydroxy(2-(pivaloylthio)ethoxy)phosphoryl)methyl)benzo[b]thiophene-2-carboxylate Silver(I) (difluoro(2-((perfluorophenoxy)carbonyl)benzo[b]thiophen-5-yl)methyl)phosphonate was Synthesized Starting from (difluoro(2-((perfluorophenoxy)carbonyl)benzo[b]thiophen-5-yl)methyl)phosphonic Acid Using the Method Described in Step 1, Method 3 for the Synthesis of silver(I) ((2-((perfluorophenoxy)carbonyl)benzo[b]thiophen-5-yl)methyl)phosphonate

To a suspension of silver(I) ((2-((perfluorophenoxy)carbonyl)benzo[b]thiophen-5-yl)methyl)phosphonate (647 mg, 940 μmol, 1.0 eq) in anhydrous toluene (10 mL) was added in a dropwise manner S-(2-iodoethyl) 2,2-dimethylpropanethioate (310 mg, 1.14 mmol, 1.2 eq). After complete addition of the alcohol, the resulting mixture was stirred at room temperature for an additional 12 h. The heterogeneous mixture was filtered, and the filtrate was concentrated in vacuo. The resulting residue was purified by reverse phase chromatography to give perfluorophenyl 5-(difluoro(hydroxy(2-(pivaloylthio)ethoxy)phosphoryl)methyl)benzo[b]thiophene-2-carboxylate (240 mg, 388 μmol, 41% yield). LCMS (ESI) m/z=617 [M−H]⁻.

### Step 2: Preparation of Perfluorophenyl 5-(((2-(butyrylthio)ethoxy)(2-(pivaloylthio)ethoxy)phosphoryl)difluoromethyl)benzo[b]thiophene-2-carboxylate

Perfluorophenyl 5-(((2-(butyrylthio)ethoxy)(2-(pivaloylthio)ethoxy)phosphoryl)difluoromethyl)benzo[b]thiophene-2-carboxylate was synthesized using similar protocol outlined above. Starting with perfluorophenyl 5-(difluoro(hydroxy(2-(pivaloylthio)ethoxy)phosphoryl)methyl)benzo[b]thiophene-2-carboxylate (190 mg, 307 μmol, 1.0 eq), AgNO₃ (207 mg, 1.22 mmol, 4.0 eq), and S-(2-iodoethyl) butanethioate (94.9 mg, 368 μmol, 1.2 eq) produced perfluorophenyl 5-(((2-(butyrylthio)ethoxy)(2-(pivaloylthio)ethoxy)phosphoryl)difluoromethyl)benzo[b]thiophene-2-carboxylate (55.0 mg, 73.4 μmol, 24%) as a white solid. LCMS (ESI) m/z=749 [M+H]⁺.

The following intermediates in Table 8 were prepared using a similar protocol described above for synthesis of perfluorophenyl 5-(((2-(butyrylthio)ethoxy)(2-(pivaloylthio)ethoxy)phosphoryl)difluoromethyl)benzo[b]thiophene-2-carboxylate and utilizing the appropriate advanced intermediate(s) as starting material(s).

TABLE 8

| Name | Structure | LCMS | NMR |
|---|---|---|---|
| perfluorophenyl 5- (difluoro ((((isopropoxycarbonyl) oxy) methoxy)(2-((3- methylbutanoyl) thio)ethoxy) phosphoryl)methyl) benzo[b]thiophene-2- carboxylate | 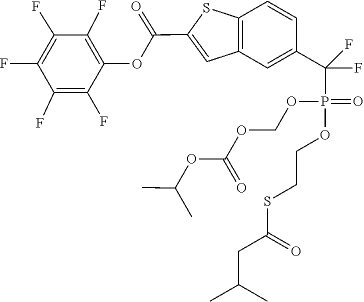 [SMILES: CC(C)CC(=O)SCCOP(=O)(OCOC(=O)OC(C)C)C(F)(F)c1ccc2sc(C(=O)Oc3c(F)c(F)c(F)c(F)c3F)cc2c1] | 757 [M + Na]⁺ | ¹H NMR (400 MHZ, CDCl₃) δ 8.40 (s, 1H), 8.24 (s, 1H), 8.03 (d, J = 8.6 Hz, 1H), 7.77 (d, J = 8.7 Hz, 1H), 5.69 (ddd, J = 17.8, 12.2, 5.2 Hz, 2H), 4.93 (dt, J = 12.5, 6.3 Hz, 1H), 4.34- 4.14 (m, 2H), 3.14 (td, J = 6.6, 3.0 Hz, 2H), 2.42 (d, J = 7.1 Hz, 2H), 2.13 (dt, J = 13.7, 6.8 Hz, 1H), 1.33 (s, 3H), 1.31 (s, 3H), 0.95 (s, 3H), 0.93 (s, 3H) |

### Preparation of 5-(((((S)-1-isopropoxy-1-oxopropan-2-yl)amino)(phenoxy)phosphoryl)methyl)benzo[b]thiophene-2-carboxylic Acid

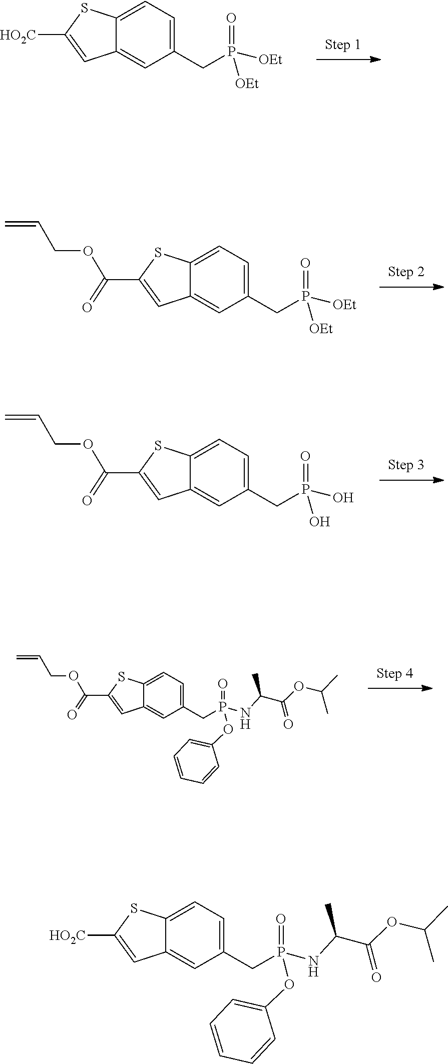 [SMILES: CCOP(=O)(Cc1ccc2sc(C(=O)O)cc2c1)OCC ; C=CCOC(=O)c1cc2cc(CP(=O)(OCC)OCC)ccc2s1 ; C=CCOC(=O)c1cc2cc(CP(=O)(O)O)ccc2s1 ; C=CCOC(=O)c1cc2cc(CP(=O)(N[C@@H](C)C(=O)OC(C)C)Oc3ccccc3)ccc2s1 ; CC(C)OC(=O)[C@H](C)NP(=O)(Cc1ccc2sc(C(=O)O)cc2c1)Oc1ccccc1]

### Step 1: Preparation of Allyl 5-((diethoxyphosphoryl)methyl)benzo[b]thiophene-2-carboxylate

To a suspension of 5-((diethoxyphosphoryl)methyl)benzo[b]thiophene-2-carboxylic acid (1.0 g, 3.0 mmol, 1.0 eq) and K₂CO₃ (839 mg, 6.1 mmol, 2.0 eq) in DMF (20 mL) was added 3-bromoprop-1-ene (440 mg, 3.6 mmol, 1.2 eq). The mixture was stirred at room temperature for 14 h and poured over water (30 mL). The mixture was extracted with EtOAc (25 mL×3). The combined organic layers were dried over Na₂SO₄, filtered, and concentrated under reduced pressure, the residue was purified by column chromatography to give allyl 5-((diethoxyphosphoryl)methyl)benzo[b]thiophene-2-carboxylate (0.980 g, 2.7 mmol, 88% yield) as a light-yellow solid. LCMS (ESI) m/z=369 [M+H]⁺; ¹H NMR (400 MHz, CDCl₃) δ 8.04 (s, 1H), 7.84-7.77 (m, 2H), 7.45-7.37 (m, 1H), 6.18-5.94 (m, 1H), 5.49-5.39 (m, 1H), 5.36-5.28 (m, 1H), 4.87-4.83 (m, 2H), 4.08-3.97 (m, 4H), 3.27 (d, J=21.4 Hz, 2H), 1.25 (t, J=7.1 Hz, 6H).

### Step 2: Preparation of ((2-((allyloxy)carbonyl)benzo[b]thiophen-5-yl)methyl)phosphonic Acid

To a solution of allyl 5-((diethoxyphosphoryl)methyl)benzo[b]thiophene-2-carboxylate (980 mg, 2.7 mmol, 1.0 eq) in CH₂Cl₂ (15 mL) was added bromotrimethylsilane (3 mL). The mixture was stirred at room temperature for 14 h and subsequently concentrated under reduced pressure. The resulting residue was triturated with H₂O (5 mL) and the resulting precipitates were filtered. The filter cake was washed with H₂O (5 mL×2) and dried under reduced pressure to give ((2-((allyloxy)carbonyl)benzo[b]thiophen-5-yl)methyl)phosphonic acid (0.710 g, 2.3 mmol, 86% yield) as a white solid. LCMS (ESI) m/z=313 [M+H]⁺; ¹H NMR (400 MHz, DMSO-d₆) δ 8.20 (s, 1H), 7.96 (d, J=8.4 Hz, 1H), 7.92-7.83 (m, 1H), 7.50-7.38 (m, 1H), 6.12-5.98 (m, 1H), 5.46-5.38 (m, 1H), 5.32-5.27 (m, 1H), 4.85-4.80 (m, 2H), 3.08 (d, J=21.2 Hz, 2H).

### Step 3: Preparation of Allyl 5-(((((S)-1-isopropoxy-1-oxopropan-2-yl)amino)(phenoxy)phosphoryl)methyl)benzo[b]thiophene-2-carboxylate

To a cooled (0° C.) solution (under a constant stream of N₂ (g)) of ((2-((allyloxy)carbonyl)benzo[b]thiophen-5-yl)methyl)phosphonic acid (2.80 g, 8.96 mmol, 1 eq) and catalytic DMF (1 drop) in dry CH₂Cl₂ (50 mL) was added oxalyl chloride (3.40 g, 26.8 mmol, 3 eq). After effervescence of gas ceased, the mixture was warmed at 40° C. After 2 h, the mixture was cooled to room temperature and concentrated in vacuo to give yellow solids. The solids were subsequently diluted CH₂Cl₂ (50 mL) and cooled to 0° C. To the cooled solution was added phenol (0.843 g, 8.96 mmol, 1 eq) and Et₃N (4.53 g, 44.8 mmol, 5 eq). After complete addition, the mixture was warmed to room temperature and stirred for 1 h, followed by introduction of propan-2-yl (2S)-2-aminopropanoate (1.75 g, 13.4 mmol, 1.5 eq) to the mixture. After stirring for an additional 2 h, the mixture was concentrated to dryness.

The residue was purified by C18 column (elution 50%-80% acetonitrile in water) to give allyl 5-(((((S)-1-isopropoxy-1-oxopropan-2-yl)amino)(phenoxy)phosphoryl)methyl)benzo[b]thiophene-2-carboxylate (2.23 g, 4.44 mmol, 49.6% yield) as white solids. LCMS (ESI) m/z=502.0 [M+H]⁺; ¹H NMR (400 MHz, CDCl₃) δ 8.05 (d, J=6.9 Hz, 1H), 7.91-7.80 (m, 2H), 7.53-7.43 (m, 1H), 7.29 (d, J=8.1 Hz, 2H), 7.18-7.09 (m, 3H), 6.05 (ddd, J=16.1, 10.9, 5.6 Hz, 1H), 5.48-5.40 (m, 1H), 5.32 (dd, J=10.4, 1.2 Hz, 1H), 4.98-4.87 (m, 1H), 4.85 (d, J=5.7 Hz, 2H), 4.04-3.85 (m, 1H), 3.44 (dd, J=20.7, 14.1 Hz, 2H), 3.12 (t, J=10.9 Hz, 1H), 1.21-1.10 (m, 9H).

### Step 4: Preparation of 5-(((((S)-1-isopropoxy-1-oxopropan-2-yl)amino)(phenoxy)phosphoryl)methyl)benzo[b]thiophene-2-carboxylic Acid

A solution of allyl 5-(((((S)-1-isopropoxy-1-oxopropan-2-yl)amino)(phenoxy)phosphoryl)methyl)benzo[b]thiophene-2-carboxylate (90 mg, 0.1794 mmol, 1 eq), pyrrolidine (12.7 mg, 179 μmol, 1 eq), Pd(PPh₃)₄ (10.3 mg, 8.97 μmol, 0.05 eq) in CH₂Cl₂ (5 mL) was stirred under N₂ (g). After 2 h, the reaction was concentrated in vacuo. The residue was purified by C18 column (elution 30%-70% acetonitrile in water) to yield 5-(((((S)-1-isopropoxy-1-oxopropan-2-yl)amino)(phenoxy)phosphoryl)methyl)benzo[b]thiophene-2-carboxylic acid (64.0 mg, 0.1386 mmol, 77.4% yield) as white solids. LCMS (ESI) m/z=462.1 [M+H]⁺.

The following intermediates in Table 9 were prepared using the described above for synthesis of 5-(((((S)-1-isopropoxy-1-oxopropan-2-yl)amino)(phenoxy)phosphoryl)methyl)benzo[b]thiophene-2-carboxylic acid and utilizing the appropriate utilizing the appropriate starting materials and modifications.

TABLE 9

| Name | Structure | LCMS | NMR |
|---|---|---|---|
| 5-(fluoro((((S)-1- isopropoxy-1- oxopropan-2- yl)amino)(phenoxy) phosphoryl)methyl) benzo[b]thiophene-2- carboxylic acid | 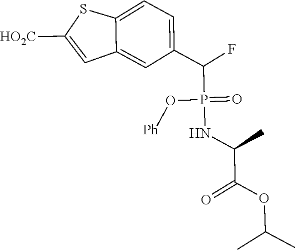 [SMILES: CC(C)OC(=O)[C@H](C)NP(=O)(Oc1ccccc1)C(F)c1ccc2sc(C(=O)O)cc2c1] | 480.1 [M + H]⁺ | |

### Synthetic Scheme: Preparation of 5-[1-(diethoxyphosphoryl)-2-hydroxyethyl]-1-benzothiophene-2-carboxylic Acid

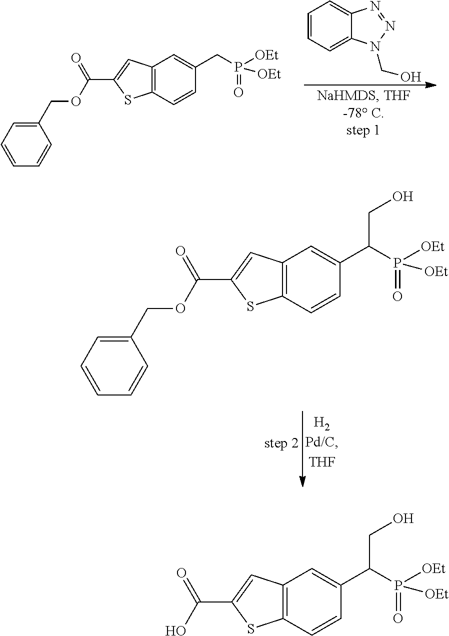 [SMILES: CCOP(=O)(Cc1ccc2sc(C(=O)OCc3ccccc3)cc2c1)OCC ; OCn1nnc2ccccc21 ; CCOP(=O)(OCC)C(CO)c1ccc2sc(C(=O)OCc3ccccc3)cc2c1 ; CCOP(=O)(OCC)C(CO)c1ccc2sc(C(=O)O)cc2c1]

### Step 1: Benzyl 5-[1-(diethoxyphosphoryl)-2-hydroxyethyl]-1-benzothiophene-2-carboxylate

To a solution of benzyl 5-[(diethoxyphosphoryl)methyl]-1-benzothiophene-2-carboxylate (200 mg, 0.4779 mmol, 1 eq) in tetrahydrofuran (10 mL) at −78° C. was added a solution of sodium bis(trimethylsilylamide) (1 M in THF) (1.43 mL, 1.43 mmol, 3.0 eq) dropwise. The reaction was stirred for 5 min. at −78° C. then 1H-benzotriazole-1-methanol (142 mg, 0.955 mmol, 2 eq) was added in one portion. The reaction was stirred at −78° C. for 2 h. The reaction mixture was quenched with saturated ammonium chloride aqueous solution (10 mL) at −78° C. then the ice-bath was removed. The product was extracted with ethyl acetate (3×30 mL). The combined extracts were dried with anhydrous sodium sulfate, filtered, and concentrated under reduced pressure. The crude residue was purified by reverse phase chromatography on a 50 g C₁₈ cartridge eluting with a gradient of 5-80% MeCN in water (with 0.1% formic acid) to give benzyl 5-[1-(diethoxyphosphoryl)-2-hydroxyethyl]-1-benzothiophene-2-carboxylate (24 mg, 0.05351 mmol, 11.2%) as a clear thick oil. LCMS: m/z=449.2 (M+H)+; ¹H NMR (400 MHz, DMSO-d₆) δ 8.27 (s, 1H), 8.02 (d, J=8.5 Hz, 1H), 7.99 (s, 1H), 5.40 (s, 2H), 4.87 (t, J=5.5 Hz, 1H), 4.07-3.94 (m, 3H), 3.93-3.71 (m, 3H), 3.52-3.41 (m, 1H), 1.21 (t, J=7.3 Hz, 3H), 1.03 (t, J=7.3 Hz, 3H).

### Step 2: 5-[1-(Diethoxyphosphoryl)-2-hydroxyethyl]-1-benzothiophene-2-carboxylic Acid

A mixture of 10% palladium on carbon (50% wet) (80 mg, 0.03758 mmol, 0.163 eq) and benzyl 5-[1-(diethoxyphosphoryl)-2-hydroxyethyl]-1-benzothiophene-2-carboxylatebenzyl 5-[1-(diethoxyphosphoryl)-2-hydroxyethyl]-1-benzothiophene-2-carboxylate (38 mg, 0.08473 mmol, 1 eq) in tetrahydrofuran (8 mL) was degassed with nitrogen for 5 min. Hydrogen was bubbled for 5 min. then the reaction was allowed to stir at room temperature under hydrogen (1 atm) for 20 h. The reaction was filtered over Celite and eluted with MeOH. The filtrate was concentrated under reduced pressure. The crude residue was purified by reverse phase chromatography on a 50 g C₁₈ cartridge eluting with 5-80% MeCN in water (containing 0.1% formic acid) then concentrated under reduced pressure and freeze dried to give 5-[1-(diethoxyphosphoryl)-2-hydroxyethyl]-1-benzothiophene-2-carboxylic acid (8.00 mg, 0.02232 mmol, 26.4%) as a white solid. LCMS: m/z=359.0 (M+H)+; ¹H NMR (400 MHz, DMSO-d₆) δ 8.02-7.92 (m, 2H), 7.90 (s, 1H), 7.48-7.42 (m, 1H), 4.92-4.79 (m, 1H), 4.08-3.69 (m, 6H), 3.52-3.39 (m, 1H), 1.21 (t, J=7.2 Hz, 3H), 1.03 (t, J=7.3 Hz, 3H).

### Synthetic scheme: Synthesis of 5-(1-(diethoxyphosphoryl)ethyl)benzo[b]thiophene-2-carboxylic Acid and 5-(2-(diethoxyphosphoryl)propan-2-yl)benzo[b]thiophene-2-carboxylic Acid

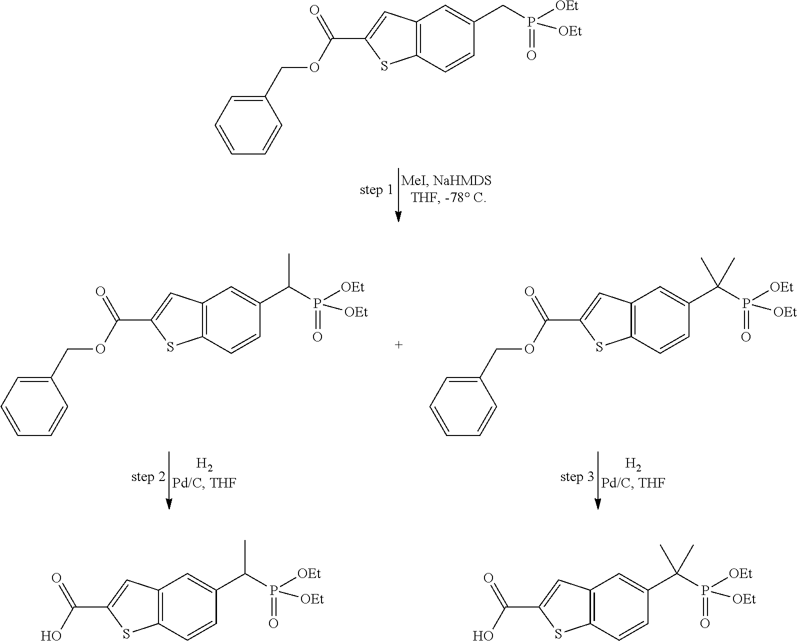 [SMILES: CCOP(=O)(Cc1ccc2sc(C(=O)OCc3ccccc3)cc2c1)OCC ; CCOP(=O)(OCC)C(C)c1ccc2sc(C(=O)OCc3ccccc3)cc2c1 ; CCOP(=O)(OCC)C(C)(C)c1ccc2sc(C(=O)OCc3ccccc3)cc2c1 ; CCOP(=O)(OCC)C(C)c1ccc2sc(C(=O)O)cc2c1 ; CCOP(=O)(OCC)C(C)(C)c1ccc2sc(C(=O)O)cc2c1]

### Step 1: rac-Benzyl 5-[1-(diethoxyphosphoryl)ethyl]-1-benzothiophene-2-carboxylate and benzyl 5-[2-(diethoxyphosphoryl)propan-2-yl]-1-benzothiophene-2-carboxylate

To a solution of benzyl 5-[(diethoxyphosphoryl)methyl]-1-benzothiophene-2-carboxylate (200 mg, 0.4779 mmol, 1 eq) and methyl iodide (88.5 μL, 1.43 mmol, 3 eq) in tetrahydrofuran (5 mL) at −78° C. was added a solution of sodium bis(trimethylsilylamide) (1 M in THF) (1.43 mL, 1.43 mmol, 3 eq) dropwise. The mixture was stirred at −78° C. for 2 h. The ice-bath was removed and the reaction was allowed to stir at room temperature for 1 h. The reaction mixture was quenched with saturated ammonium chloride aqueous solution (10 mL). The product was extracted with ethyl acetate (3×30 mL). The combined extracts were dried with anhydrous sodium sulfate, filtered and concentrated under reduced pressure. The crude residue was purified by reverse phase chromatography on a 50 g C₁₈ cartridge eluting with a gradient of 5-80% MeCN in water (with 0.1% formic acid) to give benzyl rac-5-[1-(diethoxyphosphoryl)ethyl]-1-benzothiophene-2-carboxylate (89.0 mg, 0.1993 mmol, 41.9%) as a clear oil and benzyl 5-[2-(diethoxyphosphoryl)propan-2-yl]-1-benzothiophene-2-carboxylate (22.0 mg, 0.5087 mmol, 10.6%) as a clear oil. rac-Benzyl 5-[1-(diethoxyphosphoryl)ethyl]-1-benzothiophene-2-carboxylate LCMS: m/z=433.0 (M+H)⁺. ¹H NMR (400 MHz, DMSO-d₆) δ 8.08 (s, 1H), 7.88-7.85 (m, 1H), 7.83 (d, J=8.7 Hz, 1H), 7.52-7.47 (m, 3H), 7.45-7.36 (m, 3H), 5.42 (s, 2H), 4.12-4.02 (m, 2H), 4.00-3.91 (m, 1H), 3.89-3.78 (m, 1H), 3.38-3.25 (m, 1H), 1.66 (dt, J=18.3, 7.6 Hz, 3H), 1.30 (t, J=7.3 Hz, 3H), 1.16 (t, J=7.3 Hz, 3H).

benzyl 5-[2-(diethoxyphosphoryl)propan-2-yl]-1-benzothiophene-2-carboxylate: LCMS: m/z=447.0 (M+H)⁺. ¹H NMR (400 MHz, DMSO-d₆) δ 8.01 (s, 1H), 7.93-7.90 (m, 1 J), 7.74 (d, J=8.6 Hz, 1H), 7.69-7.63 (m, 1H), 7.39 (d, J=8.6 Hz, 1H), 7.36-7.25 (m, 3H), 5.32 (s, 2H), 3.92-3.73 (m, 4H), 1.61 (d, J=16.8 Hz, 6H), 1.11 (t, J=7.2 Hz, 6H).

### Step 2: rac-5-[1-(Diethoxyphosphoryl)ethyl]-1-benzothiophene-2-carboxylic Acid

A mixture of 10% palladium on carbon (50% wet) (80 mg, 0.03758 mmol, 0.163 eq) and benzyl 5-[1-(diethoxyphosphoryl)ethyl]-1-benzothiophene-2-carboxylate (100 mg, 0.2312 mmol, 1 eq) in tetrahydrofuran (8 mL) was degassed with nitrogen for 5 min. Hydrogen was bubbled for 5 min. then the reaction was allowed to stir at room temperature under hydrogen (1 atm). The reaction was filtered over Celite and eluted with THF. The filtrate was concentrated under reduced pressure. The crude residue was purified by reverse phase chromatography on a 50 g C₁₈ cartridge eluting with a gradient of 5-80% MeCN in water (with 0.1% formic acid) to give rac-5-[1-(diethoxyphosphoryl)ethyl]-1-benzothiophene-2-carboxylic acid (51 mg, 0.1489 mmol, 65%) as a white solid. LCMS: m/z=343.0 (M+H)⁺.

### Step 3: 5-[2-(Diethoxyphosphoryl)propan-2-yl]-1-benzothiophene-2-carboxylic Acid

A mixture of 10% palladium on carbon (50% wet) (5 mg, 0.002349 mmol, eq) and 5-[2-(diethoxyphosphoryl)propan-2-yl]-1-benzothiophene-2-carboxylate (33 mg, 0.07390 mmol, 1 eq) in tetrahydrofuran (2 mL) was degassed with nitrogen for 5 min. Hydrogen was bubbled for 5 min. then the reaction was allowed to stir at room temperature under hydrogen (1 atm). The reaction was filtered on a syringe filter and eluted with MeOH. The filtrate was concentrated under reduced pressure. The crude residue was diluted in tetrahydrofuran (2 mL). 10% Palladium on carbon (50% wet) (20 mg, 0.009396 mmol, 0.13 eq) was added under nitrogen bubbling, then hydrogen was bubbled for 5 min. The reaction was stirred at room temperature overnight under hydrogen (1 atm). The reaction was filtered on a syringe filter and eluted with MeOH. The filtrate was concentrated under reduced pressure. The crude residue was diluted in tetrahydrofuran (2 mL) then 10% palladium on carbon (50% wet) (20 mg, 0.009396 mmol, 0.13 eq) was added under nitrogen bubbling, then hydrogen was bubbled for 5 min. The reaction was stirred at room temperature under hydrogen (1 atm) for 40 h. The reaction was filtered on a syringe filter and eluted with MeOH. The filtrate was concentrated under reduced pressure to give crude 5-[2-(diethoxyphosphoryl)propan-2-yl]-1-benzothiophene-2-carboxylic acid (26.3 mg, 0.0738 mmol, 99%) as a clear oil. LCMS: m/z=357.2 (M+H)⁺.

### Synthetic Scheme: Preparation of Benzyl 5-{[bis({[(2-methoxyethoxy)carbonyl]oxy}methoxy)phosphoryl]difluoromethyl}-1-benzothiophene-2-carboxylate

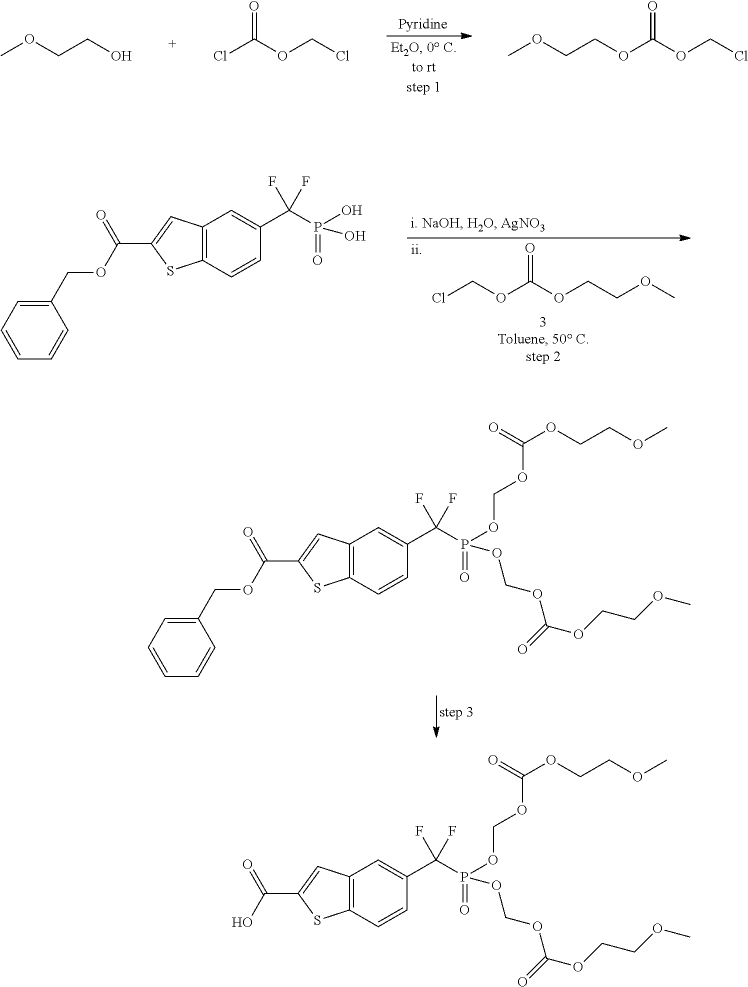

### Step 1: Chloromethyl 2-methoxyethyl Carbonate

A solution of 2-methoxyethan-1-ol (1 g, 13.1 mmol, 1 eq) and chloroacetyl chloride (1.68 g, 13.1 mmol, 1 eq) in diethyl ether (30 mL) was cooled down to 0° C. under nitrogen. Pyridine (1.04 mL, 13.1 mmol, 1.0 eq) was added dropwise and then the reaction was stirred for 15 min at 0° C. followed by 16 h at room temperature. The white suspension was filtered and rinsed with diethyl ether (30 mL). The filtrate was washed with 1 N HCl (20 mL) and water (2×20 mL) then dried over anhydrous sodium sulfate, filtered and concentrated under reduced pressure to give chloromethyl 2-methoxyethyl carbonate (1.63 g, 9.66 mmol, 74.0%) as a clear liquid.: ¹H NMR (400 MHz, CDCl₃) δ 5.76 (s, 2H), 4.42-4.37 (m, 2H), 3.68-3.64 (m, 2H), 3.42 (s, 3H).

### Step 2: Benzyl 5-{[bis({[(2-methoxyethoxy)carbonyl]oxy}methoxy)phosphoryl]difluoromethyl}-1-benzothiophene-2-carboxylate

Sodium hydroxide (59.9 mg, 1.50 mmol, 2 eq) in water (2 mL) was added dropwise to a stirred solution of ({2-[(benzyloxy)carbonyl]-1-benzothiophen-5-yl}difluoromethyl)phosphonic acid (300 mg, 0.7531 mmol, 1 eq) in water (15 mL). When the mixture reached pH˜8, silver nitrate (382 mg, 2.25 mmol, 3 eq) was added in one portion. After 2 h at room temperature, the grey suspension was cooled to 0° C. The precipitate was collected by filtration, washed with water, taken in MeCN, dried under reduced pressure and then dried under high vacuum for 2 h. The solid was suspended in dry toluene (10 mL), and chloromethyl 2-methoxyethyl carbonate (379 mg, 2.25 mmol, 3 eq) was added. The mixture was stirred for 18 h at room temperature then heated to 50° C. for 5 days. The mixture was adsorbed on silica gel and concentrated under reduced pressure. The crude residue was purified by flash-chromatography on a 24 g silica gel cartridge eluting with 0-90% EtOAc in heptane to give benzyl 5-{[bis({[(2-methoxyethoxy)carbonyl]oxy}methoxy)phosphoryl]difluoromethyl}-1-benzothiophene-2-carboxylate (290 mg, 0.4377 mmol, 58.2%) as a clear oil. LCMS: m/z=663.2 (M+H)⁺. ¹H NMR (400 MHz, CDCl₃) δ 8.17 (s, 1H), 8.16-8.14 (m, 1H), 7.98 (d, J=8.4 Hz, 1H), 7.69 (d, J=8.4 Hz, 1H), 7.51-7.48 (m, 2H), 7.46-7.36 (m, 3H), 5.75 (dd, J=12.0, 5.5, Hz, 2H), 5.69 (dd, J=12.0, 5.5 Hz, 2H), 5.43 (s, 2H), 4.36-4.31 (m, 4H), 3.64-3.60 (m, 4H), 3.39 (s, 6H).

### Step 3: Benzyl 5-{[bis({[(2-methoxyethoxy)carbonyl]oxy}methoxy)phosphoryl]difluoromethyl}-1-benzothiophene-2-carboxylate

10% Palladium on carbon (50% wet) (145 mg, 0.1353 mmol, eq) was added to a mixture of benzyl 5-{[bis({[(2-methoxyethoxy)carbonyl]oxy}methoxy)phosphoryl]difluoromethyl}-1-benzothiophene-2-carboxylate (145 mg, 0.2188 mmol, 1 eq) in anhydrous tetrahydrofuran (15 mL). Hydrogen was bubbled into the suspension for 2 min. and then the reaction mixture was stirred under hydrogen (1 atm) for 20 h. Nitrogen was bubbled and the reaction was filtered over Celite, rinsed with 2-MeTHF then concentrated under reduced pressure to give crude benzyl 5-{[bis({[(2-methoxyethoxy)carbonyl]oxy}methoxy)phosphoryl]difluoromethyl}-1-benzothiophene-2-carboxylate (144 mg, 0.2188 mmol, 99%) as a grey solid. LCMS: m/z=573.2 (M+H)⁺.

### Synthetic Scheme: Preparation of 5-{cyano[ethoxy(hydroxy)phosphoryl]methyl}-1-benzothiophene-2-carboxylic Acid

### Step 1: 5-[Cyano(diethoxyphosphoryl)methyl]-1-benzothiophene-2-carboxylate

To a solution of diethyl (cyanomethyl)phosphonate (127 mg, 722 μmol, 1.2 eq) in anhydrous 1,2-dimethoxyethane (5 mL) under nitrogen was added sodium hydride (60% in mineral oil) (50.3 mg, 1.26 mmol, 2.1 eq). The reaction was stirred at r.t. for 10 min. and then tetrakis(triphenylphosphine)palladium (34.7 mg, 30.1 μmol, 0.05 eq) and ethyl 5-iodo-1-benzothiophene-2-carboxylate (200 mg, 602 μmol, 1 eq) were added. The reaction mixture was stirred at 85° C. for 16 h. The reaction mixture was filtered over Celite (eluting with DCM) and the filtrate was concentrated under reduced pressure. The crude residue was purified by reverse phase chromatography on a 50 g C₁₈ cartridge eluting with 5-80% MeCN in water. The combined fractions were concentrated to give ethyl 5-[cyano(diethoxyphosphoryl)methyl]-1-benzothiophene-2-carboxylate (50.0 mg, 131 μmol, 21.8%) as an orange solid. LCMS: m/z=382.2 (M+H)⁺. ¹H NMR (400 MHz, DMSO-d₆) δ 8.33 (s, 1H), 8.18-8.06 (m, 2H), 7.54 (d, J=8.6 Hz, 1H), 5.67-5.56 (m, 1H), 4.37 (q, J=7.1 Hz, 2H), 4.11-3.97 (m, 4H), 1.34 (t, J=7.1 Hz, 3H), 1.20 (dt, J=9.8, 7.1 Hz, 6H).

### Step 2: 5-{Cyano[ethoxy(hydroxy)phosphoryl]methyl}-1-benzothiophene-2-carboxylic Acid

To a solution of ethyl 5-[cyano(diethoxyphosphoryl)methyl]-1-benzothiophene-2-carboxylate (80 mg, 209 μmol, 1 eq) in acetonitrile (4 mL) was added hydrochloric acid (6 mL, 3 N). The reaction mixture was stirred at 70° C. for 7 h. More hydrochloric acid (1.5 mL, 3 N) was added and the reaction mixture was stirred at 70 C for additional 22 h. Hydrochloric acid (1.5 mL, 3 N) was added and the reaction mixture was stirred at 80° C. for additional 20 h. The reaction mixture was concentrated under reduced pressure. The crude was directly purified by reverse phase chromatography using a 50 g C₁₈ cartridge eluting with a gradient of MeCN in water (5% for 3 CV then 5 to 100% in 18 CV) to give 5-{cyano[ethoxy(hydroxy)phosphoryl]methyl}-1-benzothiophene-2-carboxylic acid (52.0 mg, 159 μmol, 76.5%) as a white solid. ¹H NMR (400 MHz, DMSO-d₆) δ 8.19 (s, 1H), 8.12-7.98 (m, 2H), 7.52 (d, J=8.6 Hz, 1H), 5.23-5.12 (m, 1H), 3.97 (quin, J=7.3 Hz, 2H), 1.22-1.13 (m, 3H).

### Synthetic Scheme: Preparation of rac-5-[1-(diethoxyphosphoryl)-1-fluoroethyl]-1-benzothiophene-2-carboxylic Acid

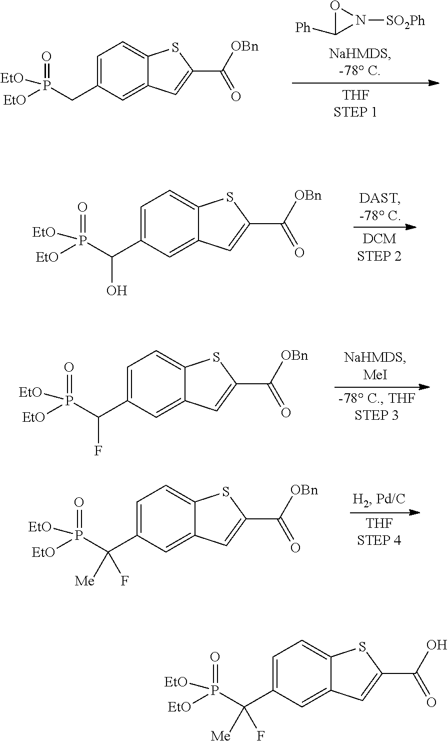

### Step 1: rac-Benzyl 5-[(diethoxyphosphoryl)(hydroxy)methyl]-1-benzothiophene-2-carboxylate

A solution of benzyl 5-[(diethoxyphosphoryl)methyl]-1-benzothiophene-2-carboxylate (2.4 g, 5.73 mmol, 1 eq) in tetrahydrofuran (75 mL) was cooled down to −78° C. then 2-(benzenesulfonyl)-3-phenyloxaziridine (2.97 g, 11.4 mmol, 2 eq) was added followed by the addition of a solution of sodium bis(trimethylsilylamide) (1.0 M in THF) (11.4 mL, 11.4 mmol, 2 eq) dropwise (internal T °=−75° C. to −69° C.). A deep purple solution was observed upon addition of base and changed rapidly to orange. The mixture was stirred for 10 min. at −78° C. The reaction mixture was quenched with saturated NH₄Cl aqueous solution (50 mL) at −78° C. then the dry ice bath was removed. EtOAc (75 mL) and water (25 mL) were added and the mixture was stirred for 30 min. (until internal temperature reached 15° C.). The phases were separated and then the aqueous layer was back-extracted with EtOAc (125 mL). The combined organic extracts were dried with anhydrous sodium sulfate, filtered, adsorbed on silica gel and concentrated under reduced pressure. The crude material was combined with another batch (6.42 mmol, 12.15 mmol in total) and was purified by flash-chromatography on a 330 g silica gel cartridge eluting with 20-100% EtOAc in heptanes to give rac-benzyl 5-[(diethoxyphosphoryl)(hydroxy)methyl]-1-benzothiophene-2-carboxylate (3.69 g, 8.49 mmol, 70%) as a white sticky solid. LCMS: m/z=869.4 (2M+H)⁺. ¹H NMR (400 MHz, CDCl₃) δ 8.10 (s, 1H), 8.04-8.00 (m, 1H), 7.88 (d, J=8.6 Hz, 1H), 7.61 (d, J=8.6 Hz, 1H), 7.51-7.47 (m, 2H), 7.46-7.35 (m, 3H), 5.42 (s, 2H), 5.17 (dd, J=10.4, 4.5 Hz, 1H), 4.18-3.95 (m, 4H), 3.10-2.99 (m, 1H), 1.33-1.20 (m, 6H).

### Step 2: Rac-Benzyl 5-[(diethoxyphosphoryl)(fluoro)methyl]-1-benzothiophene-2-carboxylate

To a solution of rac-benzyl 5-[(diethoxyphosphoryl)(hydroxy)methyl]-1-benzothiophene-2-carboxylate (1.56 g, 3.59 mmol, 1 eq) in methylene chloride (30 mL) at −78° C. under nitrogen was added (diethylamino)sulfur trifluoride (568 μL, 4.30 mmol, 1.2 eq). The reaction was stirred for 15 min. at −78° C. The reaction was quenched with the addition of a saturated sodium bicarbonate aqueous solution (50 mL) and the product was extracted with DCM (3×50 mL). The combined organic layers were dried over anhydrous sodium sulfate, filtered and concentrated under reduced pressure. The crude residue was purified by reverse phase chromatography on a 275 g C₁₈ cartridge eluting with 5-80% MeCN in water to give rac-benzyl 5-[(diethoxyphosphoryl)(fluoro)methyl]-1-benzothiophene-2-carboxylate (650 mg, 1.48 mmol, 41.6%) as a thick clear oil. LCMS: m/z=873.2 (2M+H)⁺. ¹H NMR (400 MHz, CDCl₃) δ 8.12 (s, 1H), 8.02-7.99 (m, 1H), 7.92 (d, J=8.7 Hz, 1H), 7.61 (d, J=8.7 Hz, 1H), 7.51-7.51 (m, 2H), 7.46-7.36 (m, 3H), 5.82 (dd, J=44.4, 7.5 Hz, 1H), 5.42 (s, 2H), 4.21-4.02 (m, 4H), 1.34-1.26 (m, 6H).

### Step 3: rac-Benzyl 5-[1-(diethoxyphosphoryl)-1-fluoroethyl]-1-benzothiophene-2-carboxylate

To a solution of benzyl 5-[(diethoxyphosphoryl)(fluoro)methyl]-1-benzothiophene-2-carboxylate (100 mg, 0.2291 mmol, 1 eq) and methyl iodide (42.7 μL, 687 μmol, 3 eq) in tetrahydrofuran (4 mL) at −78° C. was added a solution of sodium bis(trimethylsilylamide) (1 M in THF) (458 μL, 458 μmol, 2 eq) dropwise. The mixture was stirred at −78° C. for 5 min. The reaction was quenched with 1 M HCl (10 mL), warmed up to room temperature and extracted with DCM (2×25 mL). The combined organic extracts were dried over anhydrous sodium sulfate, filtered and concentrated under reduced pressure. The crude material was purified by reverse phase chromatography on 50 g C₁₈ cartridge eluting with 5-80% MeCN in water (containing 0.1% formic acid). The fractions were combined, concentrated under reduced pressure then freeze dried to give rac-benzyl 5-[1-(diethoxyphosphoryl)-1-fluoroethyl]-1-benzothiophene-2-carboxylate (67.0 mg, 0.1487 mmol, 65.0%) as a yellowish oil. LCMS: m/z=451.2 (M+H)⁺. ¹H NMR (400 MHz, CDCl₃) δ 8.13 (s, 1H), 8.05-8.02 (m, 1H), 7.90 (d, J=8.7 Hz, 1H), 7.65 (d, J=8.7 Hz, 1H), 7.52-7.47 (m, 2H), 7.46-7.36 (m, 3H), 5.42 (s, 2H), 4.27-4.16 (m, 2H), 4.05-3.94 (m, 1H), 3.90-3.79 (m, 1H), 2.03 (dd, J=25.0, 14.0 Hz, 3H), 1.37 (t, J=7.1 Hz, 3H), 1.17 (t, J=7.1 Hz, 3H).

### Step 4: rac-5-[1-(Diethoxyphosphoryl)-1-fluoroethyl]-1-benzothiophene-2-carboxylic Acid

A mixture of 10% palladium on carbon (50% wet) (99 mg, 0.04651 mmol, 0.21 eq) and benzyl 5-[1-(diethoxyphosphoryl)-1-fluoroethyl]-1-benzothiophene-2-carboxylatebenzyl 5-[1-(diethoxyphosphoryl)-1-fluoroethyl]-1-benzothiophene-2-carboxylate (99 mg, 0.2197 mmol, 1 eq) in tetrahydrofuran (6 mL) was degassed with nitrogen for 5 min. Hydrogen was bubbled for 5 min. and then the reaction was allowed to stir at room temperature under hydrogen (1 atm) for 20 h. The reaction was filtered over a syringe filter and rinsed with THF. The filtrate was concentrated under reduced pressure to give crude rac-5-[1-(diethoxyphosphoryl)-1-fluoroethyl]-1-benzothiophene-2-carboxylic acid (78.9 mg, 0.2197 mmol, 100%) as a clear oil. LCMS: m/z=361.2 (M+H)⁺.

### Synthetic Scheme: Preparation of rac-7-[(diethoxyphosphoryl)(fluoro)methyl]naphthalene-2-carboxylic Acid

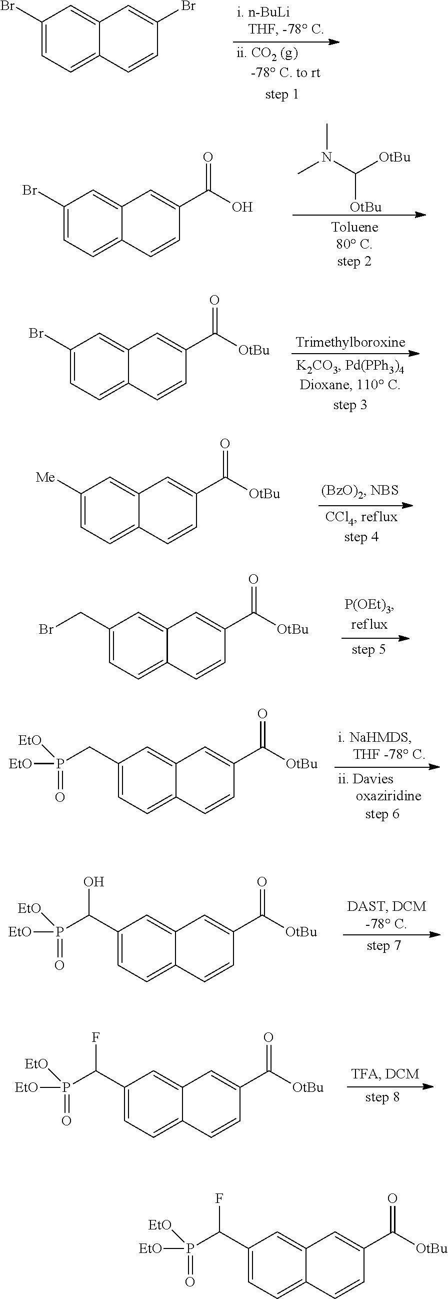

### Step 1: 7-Bromonaphthalene-2-carboxylic Acid

A solution of 2,7-dibromonaphthalene (2 g, 6.99 mmol, 1 eq) in tetrahydrofuran (24 mL) was cooled down to −78° C. under nitrogen. A solution of n-butyllithium (1.6 M in hexanes) (4.58 mL, 7.33 mmol, 1.05 eq) was added dropwise. The reaction was stirred for 15 min. at −78° C. then CO₂ was bubbled into the reaction mixture. The ice-bath was removed and the reaction was stirred for 1 h under CO₂ bubbling. The reaction was quenched with the addition of 1 N HCl (50 mL, pH=2) and then the product was extracted with EtOAc (3×75 mL). The combined organic layers were washed with water (50 mL), brine (50 mL) then dried over anhydrous sodium sulfate, filtered and concentrated under reduced pressure. The crude residue was triturated in heptanes and the resulting solid was filtered and dried to give 7-bromonaphthalene-2-carboxylic acid (1.27 g, 5.05 mmol, 72.5%) as a white solid. ¹H NMR (400 MHz, CDCl₃) δ 13.21 (s, 1H), 8.61 (s, 1H), 8.44 (s, 1H), 8.08-7.96 (m, 3H), 7.81-7.76 (m, 1H).

### Step 2: Tert-Butyl 7-bromonaphthalene-2-carboxylate

To a stirring suspension of 7-bromonaphthalene-2-carboxylic acid (1 g, 3.98 mmol, 1 eq) in toluene (9 mL) at 90° C. was added N,N-dimethylformamide dibutyl acetal (3.80 mL, 15.9 mmol, 4 eq) over 15 min. The reaction was stirred for 2 h at 90° C. then an additional amount of N,N-dimethylformamide dibutyl acetal (0.9 mL, 3.38 mmol, 1 eq) was added dropwise and the reaction was stirred for 30 min. at 90° C. The reaction was cooled down to room temperature and stirred overnight. The reaction was adsorbed on silica gel and concentrated under reduced pressure. The crude residue was purified by flash-chromatography on 40 g silica gel cartridge eluting with a gradient of 0-10% EtOAc in heptanes to give tert-butyl 7-bromonaphthalene-2-carboxylate (1.11 g, 3.61 mmol, 90.9%) as a white solid. ¹H NMR (400 MHz, CDCl₃) δ 8.46 (s, 1H), 8.14 (s, 1H), 8.06 (dd, J=8.6, 1.6 Hz, 1H), 7.86 (d, J=8.6 Hz, 1H), 7.77 (d, J=8.6, Hz, 1H), 7.66 (dd, J=8.6, 1.6 Hz, 1H), 1.67 (s, 9H).

### Step 3: Tert-Butyl 7-methylnaphthalene-2-carboxylate

A mixture of tert-butyl 7-bromonaphthalene-2-carboxylate (1.11 g, 3.61 mmol, 1 eq), trimethylboroxine (906 mg, 7.22 mmol, 2 eq), tetrakis(triphenylphosphine)palladium (834 mg, 722 μmol, 0.2 eq) and potassium carbonate (1.99 g, 14.4 mmol, 4 eq) in 1,4-dioxane (20 mL) was degassed for 5 min. and then sealed and heated to 110° C. for 20 h. The reaction was diluted with EtOAc (20 mL) and adsorbed on silica gel. The crude residue was purified by flash-chromatography eluting with 0-10% EtOAc in heptanes to give tert-butyl 7-methylnaphthalene-2-carboxylate (808 mg, 3.33 mmol, 92.4%) as a white crystalline solid. ¹H NMR (400 MHz, CDCl₃) δ 8.45 (s, 1H), 7.95 (d, J=8.7, Hz, 1H), 7.80 (d, J=8.5, Hz, 1H), 7.77 (d, J=8.5 Hz, 1H), 7.71 (s, 1H), 7.40 (d, J=8.7 Hz, 1H), 2.53 (s, 3H), 1.65 (s, 9H).

### Step 4: Tert-Butyl 7-(bromomethyl)naphthalene-2-carboxylate

To a solution of tert-butyl 7-methylnaphthalene-2-carboxylate (808 mg, 3.33 mmol) in anhydrous carbon tetrachloride (25 mL) under nitrogen atmosphere was added N-bromosuccinimide (621 mg, 3.49 mmol) and benzoyl peroxide (32.2 mg, 133 μmol). The reaction mixture was heated at reflux and stirred at this temperature for 20 h. The precipitate was filtered off, washed with carbon tetrachloride (10 mL) and then the filtrate was adsorbed on silica gel and concentrated under reduced pressure. The crude material was purified by flash-chromatography on a 80 g silica gel cartridge eluting with 0-10% EtOAc in heptanes to give tert-butyl 7-(bromomethyl)naphthalene-2-carboxylate (720 mg, 2.24 mmol, 67.9%) as a white solid which contained residual starting material (roughly 25%). ¹H NMR (400 MHz, CDCl₃) δ 8.50 (s, 1H), 8.04 (dd, J=8.6, 1.6 Hz, 1H), 7.93 (s, 1H), 7.85 (t, J=8.8 Hz, 2H), 7.60 (dd, J=8.6, 1.6 Hz, 1H), 4.67 (s, 2H), 1.64 (s, 9H).

### Step 5: Tert-Butyl 7-[(diethoxyphosphoryl)methyl]naphthalene-2-carboxylate

tert-Butyl 7-(bromomethyl)naphthalene-2-carboxylate (720 mg, 2.24 mmol) was suspended in triethyl phosphite (2 mL, 11.6 mmol) and the reaction mixture was heated for 1.5 h at reflux (became a solution once at 110° C.). The reaction was cooled down to room temperature and directly purified through reverse phase chromatography on a 50 g C₁₈ cartridge using a gradient of 5-80% MeCN in water to give tert-butyl 7-[(diethoxyphosphoryl)methyl]naphthalene-2-carboxylate (580 mg, 1.53 mmol, 68.4%) as a thick yellowish oil. LCMS: m/z=379.3 (M+H)⁺. ¹H NMR (400 MHz, CDCl₃) δ 8.52 (s, 1H), 8.08-8.00 (m, 1H), 7.89-7.83 (m, 3H), 7.58-7.54 (m, 1H), 4.11-3.97 (m, 4H), 3.36 (d, J=21.6 Hz, 2H), 1.67 (s, 9H), 1.26 (t, J=7.0 Hz, 6H).

### Step 6: Rac-Tert-Butyl 7-[(diethoxyphosphoryl)(hydroxy)methyl]naphthalene-2-carboxylate

To a solution of tert-butyl 7-[(diethoxyphosphoryl)methyl]naphthalene-2-carboxylate (200 mg, 0.5285 mmol, 1 eq) in tetrahydrofuran (5 mL) at −78° C. was added a solution of sodium bis(trimethylsilylamide) (1 M in THF) (792 μL, 792 μmol, 1.5 eq) dropwise. The mixture was stirred for 2 min. and then 2-(benzenesulfonyl)-3-phenyloxaziridine (274 mg, 1.05 mmol, 2.0 eq) was added portionwise. The dark red solution was stirred at −78° C. for 20 min. The reaction mixture was quenched with saturated ammonium chloride aqueous solution (50 mL) at −78° C. and then the ice-bath was removed. EtOAc (25 mL) and water (25 mL) were added and the mixture was stirred for 30 min. The phases were separated and then the aqueous layer was back-extracted with EtOAc (2×25 mL). The combined organic extracts were dried with anhydrous sodium sulfate, filtered and concentrated under reduced pressure. The crude material was purified by flash-chromatography on a 24 g silica gel cartridge eluting with 20-100% EtOAc in heptanes to give rac-tert-butyl 7-[(diethoxyphosphoryl)(hydroxy)methyl]naphthalene-2-carboxylate (140 mg, 0.3549 mmol, 67.3%) as a white sticky solid. LCMS: m/z=394.6 (M+H)⁺. ¹H NMR (400 MHz, CDCl₃) δ 8.57 (s, 1H), 8.08 (s, 1H), 8.05 (d, J=8.6, Hz, 1H), 7.90 (d, J=8.3 Hz, 1H), 7.87 (d, J=8.3 Hz, 1H), 7.72 (d, J=8.3 Hz, 1H), 5.24 (dd, J=11.1, 4.7 Hz, 1H), 4.18-3.99 (m, 4H), 3.31 (dd, J=11.1, 4.7 Hz, 1H), 1.66 (s, 9H), 1.32-1.22 (m, 6H).

### Step 7: Rac-Tert-Butyl 7-[(diethoxyphosphoryl)(fluoro)methyl]naphthalene-2-carboxylate

To a solution of tert-butyl 7-[(diethoxyphosphoryl)(hydroxy)methyl]naphthalene-2-carboxylate (140 mg, 0.3549 mmol, 1 eq) in methylene chloride (5 mL) at −78° C. under nitrogen was added a solution of (diethylamino)sulfur trifluoride (56.1 μL, 425 μmol, 1.2 eq) dropwise. The reaction was stirred for 20 min at −78° C. The reaction was quenched with the addition of saturated sodium bicarbonate aqueous solution (50 mL) and extracted with DCM (3×50 mL). The combined organic layers were dried over anhydrous sodium sulfate, filtered and concentrated under reduced pressure. The crude residue was purified by reverse phase chromatography on a 50 g C₁₈ cartridge eluting with a gradient of 5-80% MeCN in water to give rac-tert-butyl 7-[(diethoxyphosphoryl) (fluoro)methyl]naphthalene-2-carboxylate (80.0 mg, 0.2018 mmol, 57.1%) as a clear oil. LCMS: m/z=397.4 (M+H)⁺. ¹H NMR (400 MHz, CDCl₃) δ 8.59 (s, 1H), 8.11-8.04 (m, 2H), 7.94 (d, J=8.3, Hz, 1H), 7.90 (d, J=8.3 Hz, 1H), 7.72 (d, J=8.6, 1H), 5.90 (dd, J=44.6, 8.1 Hz, 1H), 4.24-4.02 (m, 4H), 1.35-1.25 (m, 6H).

### Step 8: rac-7-[(Diethoxyphosphoryl)(fluoro)methyl]naphthalene-2-carboxylic Acid

To a solution of rac-tert-butyl 7-[(diethoxyphosphoryl)(fluoro)methyl]naphthalene-2-carboxylate (80 mg, 0.2018 mmol) in methylene chloride (4 mL) was added trifluoroacetic acid (1 mL). The resulting yellow solution was stirred for 2.5 h at room temperature. The reaction was concentrated under reduced pressure to give crude rac-7-[(diethoxyphosphoryl)(fluoro)methyl]naphthalene-2-carboxylic acid (68.3 mg, 0.2018 mmol, 1 eq) as a clear oil. LCMS: m/z=341.2 (M+H)⁺.

### Synthetic Scheme: Preparation of 5-{[bis({[(2S)-2-(methoxycarbonyl)pyrrolidine-1-carbonyloxy]methoxy})phosphoryl]difluoromethyl}-1-benzothiophene-2-carboxylic Acid

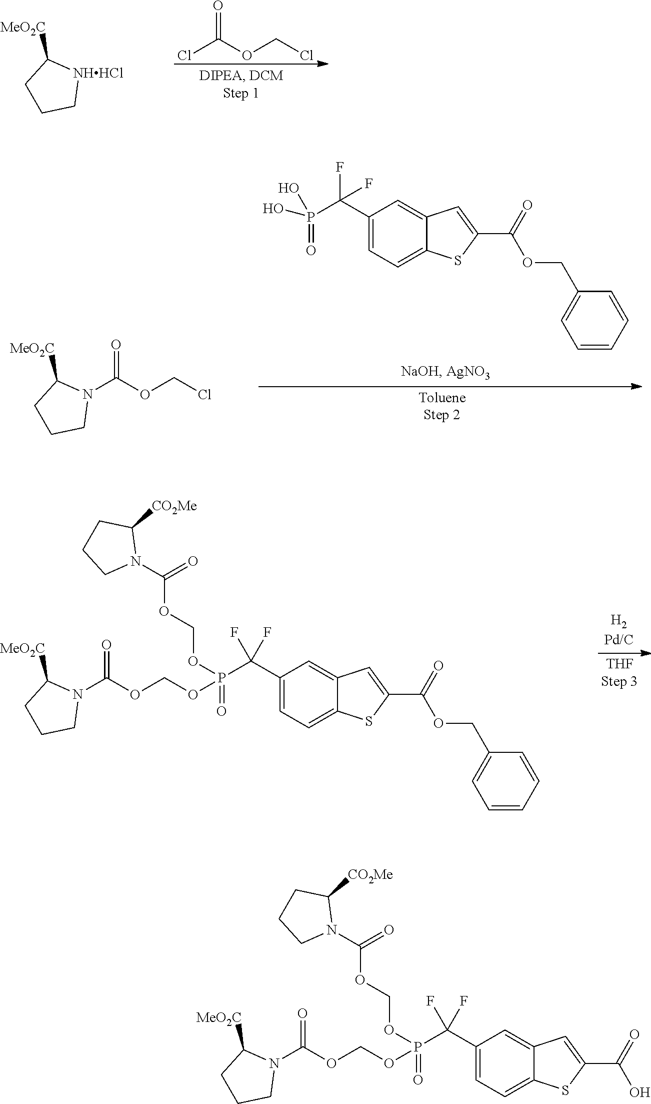

### Step 1: 1-Chloromethyl 2-methyl (2S)-pyrrolidine-1,2-dicarboxylate

Chloroacetyl chloride (777 mg, 6.03 mmol) was added to a solution of methyl (2S)-pyrrolidine-2-carboxylate hydrochloride (1.00 g, 6.03 mmol) and N,N-diisopropylethylamine (818 mg, 6.33 mmol, 1.05 eq) in dichloromethane (15 mL) at 0° C. under nitrogen atmosphere. After stirring for 0.5 h, the reaction mixture was washed successively with 1 N aqueous hydrochloric acid (50 mL), water (50 mL) and brine. The organic layer was dried over anhydrous sodium sulfate, filtered and concentrated under reduced pressure. The product was purified by flash-chromatography using a 25 g silica gel cartridge eluting with a gradient of ethyl acetate in heptanes (0 to 50%). The combined fractions were concentrated under reduced pressure to afford 1-chloromethyl 2-methyl (2S)-pyrrolidine-1,2-dicarboxylate (701 mg, 3.16 mmol, 52.7%). ¹H NMR: (400 MHz, CDCl₃) δ 5.88 (d, J=6.0 Hz, 0.5H), 5.76-5.70 (m, 1H), 5.65 (d, J=6.2 Hz, 0.5H), 4.39 (ddd, J=18.8, 8.5, 3.6 Hz, 1H), 3.74 (d, J=3.4 Hz, 3H), 3.68-3.45 (m, 2H), 2.32-2.16 (m, 1H), 2.10-1.87 (m, 3H).

### Step 2: 1-{[({2-[(Benzyloxy)carbonyl]-1-benzothiophen-5-yl}difluoromethyl)({[(2S)-2-(methoxycarbonyl)pyrrolidine-1-carbonyloxy]methoxy})phosphoryl]oxy}methyl 2-methyl (2S)-pyrrolidine-1,2-dicarboxylate

Sodium hydroxide (59.9 mg, 1.50 mmol) in water (2 mL) was added dropwise to a stirred solution of ({2-[(benzyloxy)carbonyl]-1-benzothiophen-5-yl}difluoromethyl)phosphonic acid (300 mg, 753 μmol, 1 eq) in water (15 mL) then silver nitrate (382 mg, 2.25 mmol) was added. After 2 h at r.t., the suspension was cooled to 0° C. The precipitate was collected by filtration, washed with water, co-evaporated with toluene (2×) and dried under high vacuum. The powder was suspended in dry toluene (10 mL) and 1-chloromethyl 2-methyl (2S)-pyrrolidine-1,2-dicarboxylate (582 mg, 2.63 mmol, 3.5 eq) was added. The mixture was stirred for 18 h at room temperature. The crude was directly purified by normal phase chromatography using a 12 g silica gel cartridge eluting with a gradient of ethyl acetate in heptanes (0 to 100% in 18 CV) to afford 1-{[({2-[(benzyloxy)carbonyl]-1-benzothiophen-5-yl}difluoromethyl)({[(2S)-2-(methoxycarbonyl)pyrrolidine-1-carbonyloxy]methoxy})phosphoryl]oxy}methyl 2-methyl (2S)-pyrrolidine-1,2-dicarboxylate (90.0 mg, 117 μmol, 15.5%) as a white solid. ¹H NMR: (400 MHz, CDCl₃) δ 8.20-8.12 (m, 2H), 7.97-7.91 (m, 1H), 7.69-7.64 (m, 1H), 7.48-7.33 (m, 5H), 5.79-5.66 (m, 2H), 5.66-5.52 (m, 2H), 5.41-5.38 (m, 2H), 4.40-4.31 (m, 2H), 3.75-3.65 (m, 6H), 3.63-3.40 (m, 4H), 2.25-2.11 (m, 2H), 2.04-1.81 (m, 6H).

### Step 3: 5-{[Bis({[(2S)-2-(methoxycarbonyl)pyrrolidine-1-carbonyloxy]methoxy})phosphoryl]difluoromethyl}-1-benzothiophene-2-carboxylic Acid

Palladium on carbon (10% loading, 50% wet support) (62.1 mg, 58.5 μmol, 0.5 eq) was added to a mixture of 1-{[({2-[(benzyloxy)carbonyl]-1-benzothiophen-5-yl}difluoromethyl) ({[(2S)-2-(methoxycarbonyl)pyrrolidine-1-carbonyloxy]methoxy})phosphoryl]oxy}methyl 2-methyl (2S)-pyrrolidine-1,2-dicarboxylate (90 mg, 117 μmol, 1 eq) in anhydrous tetrahydrofuran (10 mL). Hydrogen was bubbled into the suspension for 2 min. and then the reaction mixture was stirred under 1 atm (balloon) of hydrogen for 16 h. The reaction mixture was filtered on Celite and rinsed with Me-THF. The filtrate was concentrated in vacuo to give crude 5-{[bis({[(2S)-2-(methoxycarbonyl)pyrrolidine-1-carbonyloxy]methoxy})phosphoryl]difluoromethyl}-1-benzothiophene-2-carboxylic acid (95.0 mg, 140 μmol, 119%) as a colorless oil. ¹H NMR: (400 MHz, CDCl₃) δ 8.18-8.13 (m, 1H), 8.13-8.07 (m, 1H), 7.97-7.92 (m, 1H), 7.69-7.63 (m, 1H), 5.82-5.58 (m, 4H), 4.43-4.34 (m, 2H), 3.77-3.67 (m, 6H), 3.64-3.42 (m, 4H), 2.27-2.15 (m, 2H), 2.09-1.82 (m, 6H).

### Preparation of 5-(((benzylamino)((S)-1-isopropoxy-1-oxopropan-2-yl)amino)phosphoryl)methyl)benzo[b]thiophene-2-carboxylic Acid

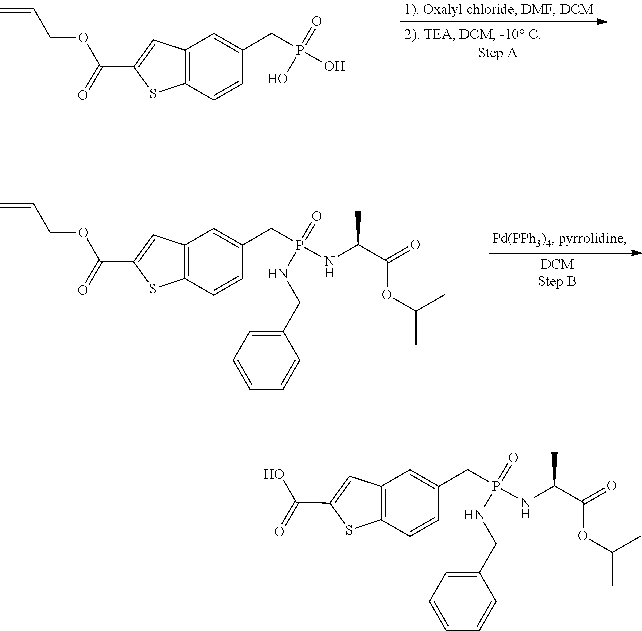 [SMILES: C=CCOC(=O)c1cc2cc(CP(=O)(O)O)ccc2s1 ; C=CCOC(=O)c1cc2cc(CP(=O)(NCc3ccccc3)N[C@@H](C)C(=O)OC(C)C)ccc2s1 ; CC(C)OC(=O)[C@H](C)NP(=O)(Cc1ccc2sc(C(=O)O)cc2c1)NCc1ccccc1]

### Step A: Allyl 5-(((benzylamino)(((S)-1-isopropoxy-1-oxopropan-2-yl)amino)phosphoryl)methyl)benzo[b]thiophene-2-carboxylate

Oxalyl chloride (814.4 mg, 6.4 mmol, 10 eq.) was added dropwise to the solution of ((2-((allyloxy)carbonyl)benzo[b]thiophen-5-yl)methyl)phosphonic acid (200 mg, 0.64 mmol, 1.0 eq.) in dry DCM (6 mL) and DMF (1 drop) at 0° C. The reaction was allowed to warm to 40° C., then stirred for additional 1˜2 hrs. The reaction was monitored by pipetting out a small amount of crude sample and quenching it with MeOH to ensure bis-Cl phosphoryl chloride allyl 5-((dichlorophosphoryl)methyl)benzo[b]thiophene-2-carboxylate had been formed completely (bis-methoxy phosphonate was observed by LCMS). After completion, the excess of oxalyl chloride and solvent were removed under reduced pressure, and the residue was re-dissolved in anhydrous DCM (5 mL). To the solution was then added BnNH₂ (64.8 mg, 0.64 mmol, 1.0 eq.) in anhydrous DCM (2 mL) and Et₃N (194.32 mg, 1.92 mmol, 3.0 eq.) at −40° C. The reaction was monitored by pipetting out a small amount of crude sample and quenching it with MeOH to ensure the most of the product was allyl 5-(((benzylamino)chlorophosphoryl)methyl)benzo[b]thiophene-2-carboxylate (mono-methoxy phosphonate was observed by LCMS). To the solution was then added isopropyl L-alaninate (107.2 mg, 0.64 mmol, 1.0 eq.) in anhydrous DCM (2 mL) at −40° C. The reaction was allowed to warm to room temperature and stirred for additional 2 hrs. After completion, the reaction was quenched by adding H₂O (10 mL), and extracted with DCM (10 mL×3). The organic layers were combined and washed with brine (20 mL), dried over anhydrous Na₂SO₄, and concentrated under reduced pressure. The residue was purified by flash column chromatography on silica gel to afford allyl 5-(((benzylamino)(((S)-1-isopropoxy-1-oxopropan-2-yl)amino)phosphoryl)methyl)benzo[b]thiophene-2-carboxylate (100 mg, 194.33 μmol, 30%). LCMS (ESI): m/z=515.2 [M+H]⁺.

### Step B: 5-(((benzylamino)(((S)-1-isopropoxy-1-oxopropan-2-yl)amino)phosphoryl)methyl)benzo[b]thiophene-2-carboxylic Acid

A solution of allyl 5-(((benzylamino)(((S)-1-isopropoxy-1-oxopropan-2-yl)amino)phosphoryl)methyl)benzo[b]thiophene-2-carboxylate (100 mg, 194 μmol, 1 eq.), Pd(PPh₃)₄(22.47 mg, 19.4 μmol, 0.1 eq.) and pyrrolidine (13.8 mg, 194 μmol, 1 eq.) in DCM (3 mL) was stirred at room temperature for 1 hr. After completion, the reaction mixture was concentrated under reduced pressure. The residue was purified by flash column chromatography on silica gel to afford 5-(((benzylamino)(((S)-1-isopropoxy-1-oxopropan-2-yl)amino)phosphoryl)methyl)benzo[b]thiophene-2-carboxylic acid (70 mg, 147.5 μmol, 76%). LCMS (ESI): m/z=475.1 [M+H]⁺.

### Synthetic Scheme: Preparation of 4-nitrophenyl 5-[(di{[(propoxycarbonyl)oxy]methoxy}phosphoryl)difluoromethyl]-1-benzothiophene-2-carboxylate

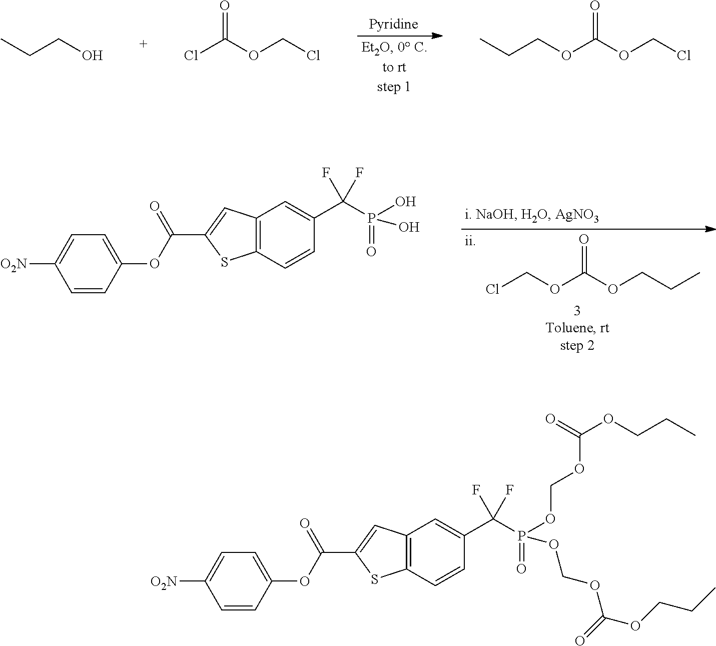

### Step 1: Chloromethyl Propyl Carbonate

A solution of propan-1-ol (1.24 mL, 16.5 mmol, 1 eq) and chloroacetyl chloride (1.46 mL, 16.5 mmol, 1 eq) in diethyl ether (50 mL) was cooled down to 0° C. under nitrogen. Pyridine (1.32 mL, 16.5 mmol, 1.0 eq) was added dropwise and then the reaction mixture was stirred for 15 min. at 0° C. followed by 3 h at room temperature. The white suspension was filtered and rinsed with diethyl ether (20 mL). The filtrate was washed with 1 N HCl (20 mL) and water (2×15 mL) and then dried over anhydrous sodium sulfate, filtered and concentrated under reduced pressure to give chloromethyl propyl carbonate (1.26 g, 8.25 mmol, 50.1%) as a clear oil: ¹H NMR (400 MHz, CDCl₃) δ 5.74 (s, 2H), 4.25-4.17 (m, 2H), 1.74 (sxt, J=7.1 Hz, 2H), 1.02-0.94 (m, 3H).

### Step 2: 4-Nitrophenyl 5-[(di{[(propoxycarbonyl)oxy]methoxy}phosphoryl)difluoromethyl]-1-benzothiophene-2-carboxylate

Sodium hydroxide (37.1 mg, 930 μmol, 2 eq) in H₂O (1 mL) was added dropwise to a stirred suspension of [difluoro({2-[(4-nitrophenoxy)carbonyl]-1-benzothiophen-5-yl})methyl]phosphonic acid (200 mg, 465 μmol, 1 eq) in water (4 mL). When the mixture reached pH˜8, product started to precipitate and silver nitrate (236 mg, 1.39 mmol, 3 eq) was added in one portion. After 2 h at 0° C., the yellow precipitate was collected by filtration, washed with water and dried under vacuum. The powder was suspended in dry toluene (10 mL), and chloromethyl propyl carbonate (282 mg, 1.85 mmol, 4 eq) was added. The mixture was stirred for 18 h at room temperature. The crude was directly purified by normal phase chromatography using a 24 g silica gel cartridge eluting with a gradient of EtOAc in heptanes (0 to 60% in 18 CV) to give 4-nitrophenyl 5-[(di{[(propoxycarbonyl)oxy]methoxy}phosphoryl)difluoromethyl]-1-benzothiophene-2-carboxylate (10 mg, 15.1 μmol, 3.25%) as a white semi-solid: LCMS: m/z=662.2 (M+H)⁺. ¹H NMR (400 MHz, DMSO-d₆) δ 8.64 (s, 1H), 8.43-8.27 (m, 4H), 7.72-7.65 (m, 3H), 5.75-5.65 (m, 4H), 4.12-4.02 (m, 4H), 1.67-1.55 (m, 4H), 0.90-0.84 (m, 6H).

### Synthetic Scheme: 4-nitrophenyl 5-({bis[({[(1-methoxy-2-methylpropan-2-yl)oxy]carbonyl}oxy)methoxy]phosphoryl}difluoromethyl)-1-benzothiophene-2-carboxylate

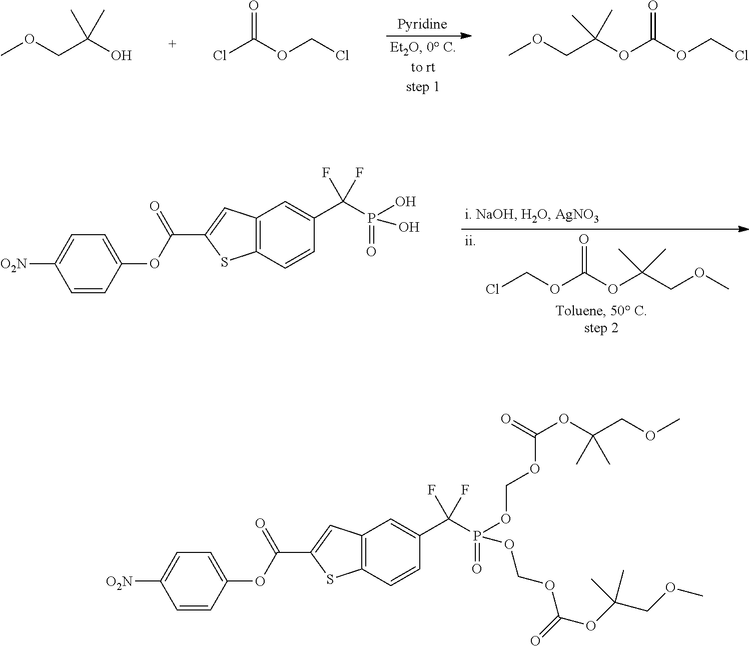

### Step 1: Chloromethyl 1-methoxy-2-methylpropan-2-yl Carbonate

A solution of 1-methoxy-2-methylpropan-2-ol (2 g, 19.2 mmol, 1 eq) and chloroacetyl chloride (1.7 mL, 19.2 mmol, 1 eq) in diethyl ether (60 mL) was cooled down to 0° C. under nitrogen. Pyridine (1.53 mL, 19.2 mmol, 1.0 eq) was added dropwise and then the reaction mixture was stirred for 15 min. at 0° C. followed by 3 h at room temperature. The white suspension was filtered and rinsed with diethyl ether (20 mL). The filtrate was washed with 1 N HCl (30 mL) and water (2×30 mL) and then dried over anhydrous sodium sulfate, filtered and concentrated under reduced pressure to give chloromethyl 1-methoxy-2-methylpropan-2-yl carbonate (860 mg, 4.37 mmol, 22.8%) as a clear oil. ¹H NMR (400 MHz, CDCl₃) δ 7.20-7.17 (m, 2H), 5.04-5.00 (m, 2H), 4.90 (s, 3H), 3.03-2.99 (m, 6H).

### Step 2: 4-Nitrophenyl 5-[(di{[(propoxycarbonyl)oxy]methoxy}phosphoryl)difluoromethyl]-1-benzothiophene-2-carboxylate

Sodium hydroxide (37.1 mg, 930 μmol, 2 eq) in H₂O (1 mL) was added dropwise to a stirred suspension of [difluoro({2-[(4-nitrophenoxy)carbonyl]-1-benzothiophen-5-yl})methyl]phosphonic acid (200 mg, 465 μmol, 1 eq) in water (5 mL). When the mixture reached pH˜8, product started to precipitate and silver nitrate (236 mg, 1.39 mmol, 3 eq) was added in one portion. After 2 h at 0° C., the white precipitate was collected by filtration, washed with water and dried under vacuum. The powder was suspended in dry toluene (10 mL), and chloromethyl 1-methoxy-2-methylpropan-2-yl carbonate (318 mg, 1.62 mmol, 3.5 eq) was added. The mixture was stirred for 18 h at 50° C. The crude mixture was directly purified by normal phase chromatography using a 24 g silica gel cartridge eluting with a gradient of EtOAc in heptanes (0 to 60% in 18 CV) to give 4-nitrophenyl 5-({bis[({[(1-methoxy-2-methylpropan-2-yl)oxy]carbonyl}oxy)methoxy]phosphoryl}difluoromethyl)-1-benzothiophene-2-carboxylate (51.0 mg, 68.0 μmol, 14.6%) as a white semi-solid. ¹H NMR (400 MHz, CDCl₃) δ 8.38-8.33 (m, 3H), 8.24 (s, 1H), 8.03 (d, J=8.8 Hz, 1H), 7.76 (d, J=8.8 Hz, 1H), 7.51-7.47 (m, 2H), 5.74-5.62 (m, 4H), 3.53-3.50 (m, 4H), 3.41-3.38 (m, 6H), 1.51 (s, 12H).

### Synthetic Scheme: Preparation of 4-nitrophenyl 5-[(di{[(dipropylcarbamoyl)oxy]methoxy}phosphoryl)difluoromethyl]-1-benzothiophene-2-carboxylate

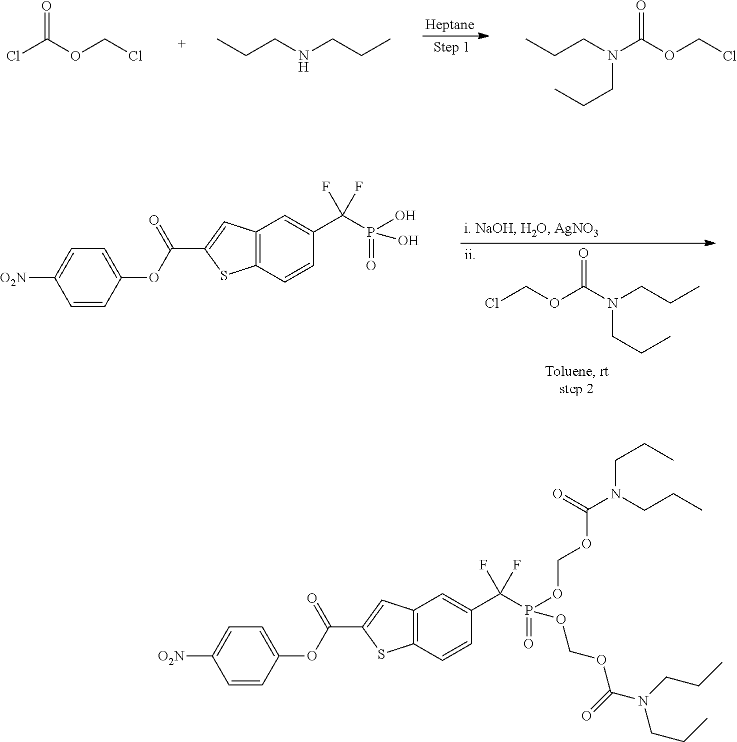

### Step 1: N,N-dipropylcarbamate

Dipropylamine (5.85 g, 57.9 mmol, 2.5 eq) was added to a solution of chloroacetyl chloride (2.06 mL, 23.2 mmol, 1 eq) in hexanes (30 mL) at 0° C. under nitrogen and stirred at the same temperature for 30 min. The reaction mixture was then diluted with ethyl acetate (50 mL). The phases were separated and the organic layer was washed successively with 1 N hydrochloric acid (50 mL), water and brine. The organic layer was dried over anhydrous sodium sulfate, then the solvent was evaporated under reduced pressure. The product was purified by flash-chromatography using a 80 g silica gel cartridge eluting with a gradient of 0-10% ethyl acetate in heptanes. The pure combined fractions were dried under reduced pressure to give chloromethyl N,N-dipropylcarbamate (2.89 g, 14.9 mmol, 64.3%) as a colorless oil. LCMS: m/z=194.2 (M+H)⁺. ¹H NMR (400 MHz, CDCl₃) δ 5.75 (s, 2H), 3.21-3.11 (m, 4H), 1.59-1.47 (m, 4H), 0.87-0.82 (in, 6H).

### Step 2: 4-Nitrophenyl 5-[(di{[(dipropylcarbamoyl)oxy]methoxy}phosphoryl)difluoromethyl]-1-benzothiophene-2-carboxylate

Sodium hydroxide (37.1 mg, 930 μmol, 2 eq) in H₂O (1 mL) was added dropwise to a stirred suspension of [difluoro({2-[(4-nitrophenoxy)carbonyl]-1-benzothiophen-5-yl})methyl]phosphonic acid (200 mg, 465 mol, 1 eq) in H₂O (5 mL). When the mixture reached pH˜8, product started to precipitate and silver nitrate (236 mg, 1.39 mmol, 3 eq) was added in one portion. After 2 h at 0° C., the yellow precipitate was collected by filtration, washed with water, dried under vacuum. The powder was suspended in dry toluene (10 mL), and chloromethyl N,N-dipropylcarbamate (313 mg, 1.62 mmol, 3.5 eq) was added. The mixture was stirred for 18 h at room temperature. The crude was directly purified by normal phase chromatography using a 12 g silica gel cartridge eluting with a gradient of EtOAc in heptanes (0 to 60% in 18 CV) to give 4-nitrophenyl 5-[(di{[(dipropylcarbamoyl)oxy]methoxy}phosphoryl)difluoromethyl]-1-benzothiophene-2-carboxylate (110 mg, 147 μmol, 31.8%) as a clear oil. ¹H NMR (400 MHz, DMSO-d₆) δ 8.65-8.56 (m, 1H), 8.41-8.21 (m, 4H), 7.76-7.59 (m, 3H), 5.77-5.60 (m, 4H), 3.17-2.99 (m, 8H), 1.53-1.38 (m, 8H), 0.85-0.75 (m, 12H).

### Synthetic Scheme: 4-nitrophenyl 5-{[bis({1-[(propoxycarbonyl)oxy]ethoxy})phosphoryl]difluoromethyl}-1-benzothiophene-2-carboxylate

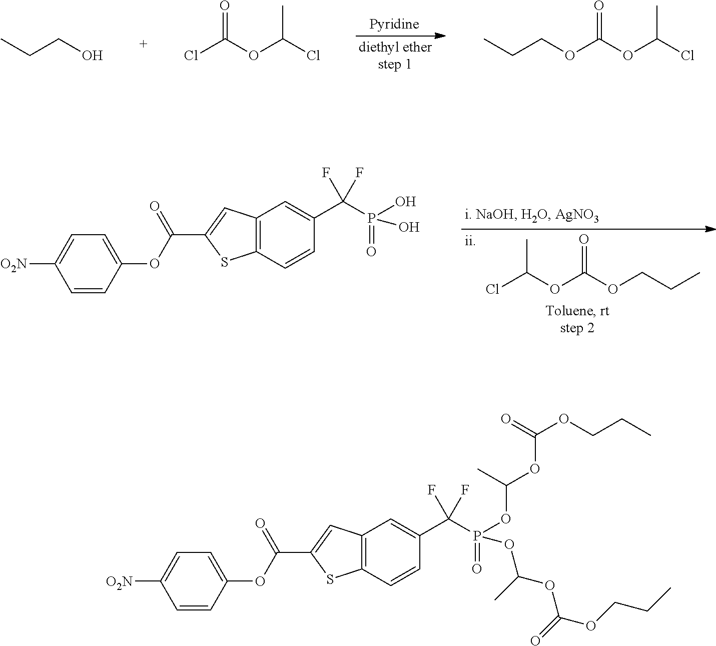

### Step 1: 1-Chloroethyl Propyl Carbonate

A solution of propan-1-ol (2 g, 33.2 mmol, 1 eq) and 1-chloroethyl chloroformate (4.74 g, 33.2 mmol, 1 eq) in diethyl ether (100 mL) was cooled to 0° C. under nitrogen. Pyridine (2.66 mL, 33.2 mmol, 1.0 eq) was added dropwise and then the reaction mixture was stirred for 15 min. at 0° C. followed by 3 h at room temperature. The white suspension was filtered and rinsed with diethyl ether (20 mL). The filtrate was washed with 1 N HCl (20 mL) and water (2×15 mL) and then dried over anhydrous sodium sulfate, filtered and concentrated under reduced pressure to give 1-chloroethyl propyl carbonate (4.62 g, 27.7 mmol, 83.5%) as a clear oil. ¹H NMR (400 MHz, CDCl₃) δ 6.44 (q, J=5.8 Hz, 1H), 4.24-4.10 (m, 2H), 1.84 (d, J=5.9 Hz, 3H), 1.78-1.66 (m, 2H), 1.01-0.94 (m, 3H).

### Step 2: 4-Nitrophenyl 5-{[bis({1-[(propoxycarbonyl)oxy]ethoxy})phosphoryl]difluoromethyl}-1-benzothiophene-2-carboxylate

Sodium hydroxide (37.1 mg, 930 μmol, 2 eq) in H₂O (1 mL) was added dropwise to a stirred suspension of [difluoro({2-[(4-nitrophenoxy)carbonyl]-1-benzothiophen-5-yl})methyl]phosphonic acid (200 mg, 465 μmol, 1 eq) in H₂O (4 mL). When the mixture became clear (pH˜8), silver nitrate (236 mg, 1.39 mmol, 2 eq) was added in one portion. After 2 h at 0° C., the yellow precipitate was collected by filtration and dried under vacuum. The powder was suspended in dry toluene (10 mL) and 1-chloroethyl propyl carbonate (269 mg, 1.62 mmol, 3.5 eq) was added. The mixture was stirred for 18 h at room temperature. The crude was directly purified by normal phase chromatography using a 12 g silica gel cartridge eluting with a gradient of EtOAc in heptanes (0 to 60% in 18 CV) to give 4-nitrophenyl 5-{[bis({1-[(propoxycarbonyl)oxy]ethoxy})phosphoryl]difluoromethyl}-1-benzothiophene-2-carboxylate (113 mg, 163 μmol, 35.3%) as a clear oil. LCMS: m/z=712.2 (M+Na)⁺.

### Preparation of (E)-3-(3-((diethoxyphosphoryl)difluoromethyl)phenyl)-2-methylacrylic Acid

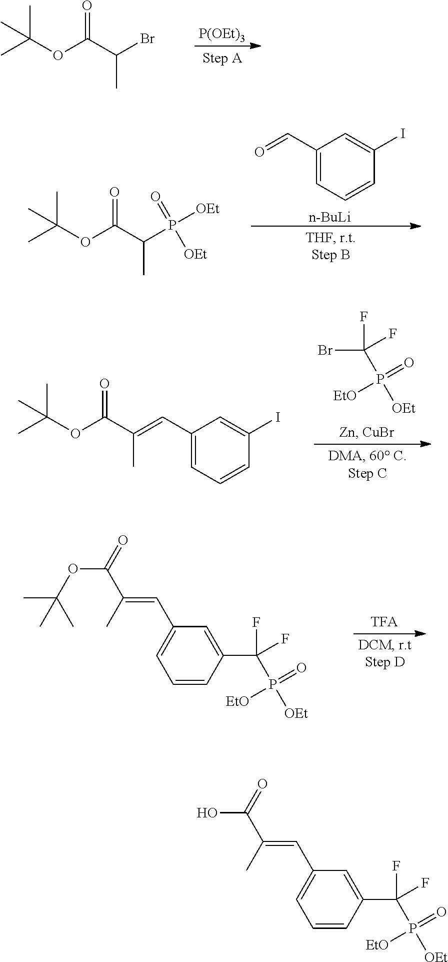

### Step A: Tert-Butyl 2-(diethoxyphosphoryl)propanoate

A mixture of tert-butyl 2-bromopropanoate (3 g, 14.3 mmol, 1 eq.) and ethan-2-ylium-1-yl diethyl phosphite (2.82 g, 17.1 mmol, 1.2 eq.) was stirred at 110° C. for 16 hrs. After completion, the reaction mixture was concentrated under reduced pressure. The residue was purified by flash column chromatography on silica gel to afford tert-butyl 2-(diethoxyphosphoryl)propanoate (3.0 g, 11.3 mmol, 79%) as a white solid. LCMS (ESI): m/z=267 [M+H]⁺.

### Step B: Tert-Butyl (E)-3-(3-iodophenyl)-2-methylacrylate

To a solution of tert-butyl 2-(diethoxyphosphoryl)propanoate (1.14 g, 4.30 mmol, 1 eq.) in THF (10 mL) was added n-BuLi (1.72 mL, 4.30 mmol, 1.0 eq.) at −78° C. under nitrogen and the resulting mixture was stirred at this temperature for 0.5 hr. Then, a solution of 3-iodobenzaldehyde (1.0 g, 4.30 mmol, 1 eq.) in THF (5 mL) was added, after addition, the reaction mixture was allowed to warm to room temperature and stirred for 14 hrs. After completion, the reaction mixture was quenched by adding H₂O (20 mL), then extracted with EtOAc (15 mL×3). The organic layers were combined and washed with brine (15 mL), dried over anhydrous Na₂SO₄, and concentrated under reduced pressure. The residue was purified by flash column chromatography on silica gel to afford tert-butyl (E)-3-(3-iodophenyl)-2-methylacrylate (739.6 mg, 2.15 mmol, 50%) as a white solid. LC-MS (ESI) m/z=345 [M+H]⁺.

### Step C: tert-butyl (E)-3-(3-((diethoxyphosphoryl)difluoromethyl)phenyl)-2-methylacrylate

A solution of diethyl (bromodifluoromethyl)phosphonate (1.08 g, 4.06 mmol, 2 eq.) zinc (265 mg, 4.06 mmol, 2 eq.) in DMAc (7 mL) was stirred at 60° C. for 1 hr under nitrogen atmosphere. Then CuBr (582 mg, 4.06 mmol, 2 eq.) was added and the mixture was stirred at 60° C. for another 1 hr. Tert-butyl (E)-3-(3-iodophenyl)-2-methylacrylate (700 mg, 2.03 mmol, 1 eq.) was added and the mixture was stirred at 60° C. for another 12 hrs. After completion, the suspension was filtered through a pad of Celite®, the filtrate was concentrated under reduced pressure. The residue was purified by flash column chromatography on silica gel to give tert-butyl (E)-3-(3-((diethoxyphosphoryl)difluoromethyl)phenyl)-2-methylacrylate (180 mg, 445 μmol, 22%) as a yellow oil. LCMS (ESI): m/z=405 [M+H]⁺.

### Step D: (E)-3-(3-((diethoxyphosphoryl)difluoromethyl)phenyl)-2-methylacrylic Acid

A solution of tert-butyl (E)-3-(3-((diethoxyphosphoryl)difluoromethyl)phenyl)-2-methylacrylate (180 mg, 445 μmol, 1 eq.) in a mixture of DCM (8 mL) and TFA (4 mL) was stirred at room temperature for 1 hr. After completion, the reaction mixture was concentrated under reduced pressure to give (E)-3-(3-((diethoxyphosphoryl)difluoromethyl)phenyl)-2-methylacrylic acid (154 mg, quant) as a white solid, which was used in next step directly without further purification. LCMS (ESI): m/z=349 [M+H]⁺.

### Preparation of (E)-3-(3-((diethoxyphosphoryl)difluoromethyl)phenyl)but-2-enoic Acid

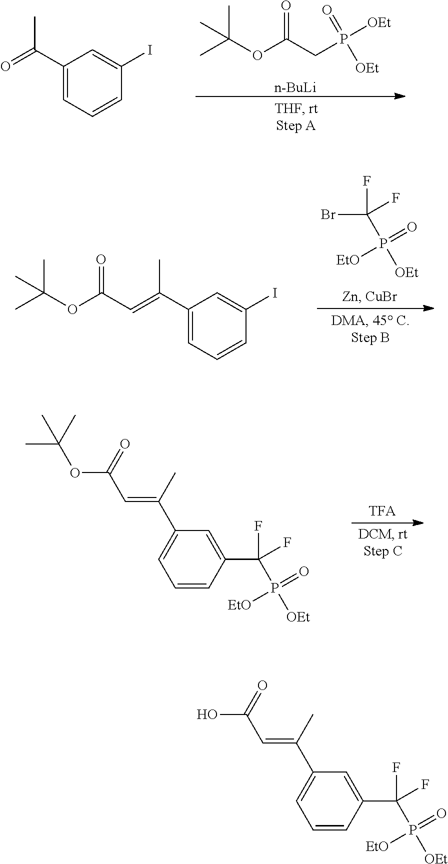

### Step A: tert-butyl (E)-3-(3-iodophenyl)but-2-enoate

n-BuLi (1.72 mL, 4.06 mmol, 1.0 eq.) was added to tert-butyl 2-(diethoxyphosphoryl)acetate (1.02 g, 4.06 mmol, 1 eq.) in THF (20 mL) at −78° C., the mixture was stirred at −78° C. for 0.5 hr. Then a solution of 1-(3-iodophenyl)ethan-1-one (1 g, 4.06 mmol, 1 eq.) in THF (5 mL) was added dropwise to the reaction. After addition, the reaction mixture was allowed to warm to room temperature and stirred for 18 hrs. After completion, the reaction mixture was quenched by adding H₂O (20 mL), then extracted with EtOAc (20 mL×3). The organic layers were combined and washed with brine (15 mL), dried over anhydrous Na₂SO₄, and concentrated under reduced pressure. The residue was purified by flash column chromatography on silica gel to afford tert-butyl (E)-3-(3-iodophenyl)but-2-enoate (800 mg, 2.32 mmol, 58%) as an oil. ¹H NMR (400 MHz, CDCl₃) δ 7.79 (t, J=1.6 Hz, 1H), 7.66 (d, J=7.9 Hz, 1H), 7.40 (d, J=7.9 Hz, 1H), 7.09 (t, J=7.9 Hz, 1H), 6.01 (d, J=1.2 Hz, 1H), 2.49 (d, J=1.2 Hz, 3H), 1.52 (s, 9H).

### Step B: Tert-Butyl (E)-3-(3-((diethoxyphosphoryl)difluoromethyl)phenyl)but-2-enoate

To a stirred suspension of Zn (301 mg, 4.64 mmol, 2 eq.) in dry DMAc (2 mL) was added slowly a solution of diethyl (bromodifluoromethyl)phosphonate (1.23 g, 4.64 mmol, 2 eq.) in DMAc (2 mL) under nitrogen atmosphere. The reaction mixture was stirred at 45° C. for 2 hrs and then CuBr (665 mg, 4.64 mmol, 2 eq.) was added and the resulting mixture was stirred at room temperature for 45 minutes. A suspension of tert-butyl (E)-3-(3-iodophenyl)but-2-enoate (800 mg, 2.32 mmol, 1 eq.) in DMAc (3 mL) was added into the reaction mixture. The mixture was stirred at 45° C. for 24 hrs under nitrogen atmosphere. The reaction mixture was then partitioned between water and ether. The mixture was passed through celite and extracted with ether. The organic extracts were washed with brine and dried over anhydrous Na₂SO₄. The solvent was removed in vacuo, and the residue was purified by flash column chromatography on silica to give tert-butyl (E)-3-(3-((diethoxyphosphoryl)difluoromethyl)phenyl)but-2-enoate (350 mg, 0.87 mmol, 37%) as an oil. ¹H NMR (400 MHz, CDCl₃) δ 7.67 (d, J=13.2 Hz, 1H), 7.63-7.54 (m, 2H), 7.45 (t, J=7.7 Hz, 1H), 6.07 (d, J=1.2 Hz, 1H), 4.27-4.11 (m, 4H), 2.54 (d, J=1.0 Hz, 3H), 1.52 (s, 9H), 1.32 (t, J=7.1 Hz, 6H).

### Step C: (E)-3-(3-((diethoxyphosphoryl)difluoromethyl)phenyl)but-2-enoic Acid

To a solution of tert-butyl (E)-3-(3-((diethoxyphosphoryl)difluoromethyl)phenyl)but-2-enoate (350 mg, 0.87 mmol, 1 eq.) in DCM (4 mL) was added TFA (6.13 g, 53.8 mmol, 62.2 eq.) at room temperature and the resulting mixture was stirred at room temperature for 18 hrs. After completion, the reaction mixture was concentrated under reduced pressure to afford (E)-3-(3-((diethoxyphosphoryl)difluoromethyl)phenyl)but-2-enoic acid (250 mg, 0.72 mmol, 83%) as an oil, which was used in next step without further purification. LC-MS (ESI) m/z=347.2 [M−H]⁻

### Preparation of Perfluorophenyl 5-((ethoxy(hydroxy)phosphoryl)difluoromethyl)-6-fluorobenzo[b]thiophene-2-carboxylate

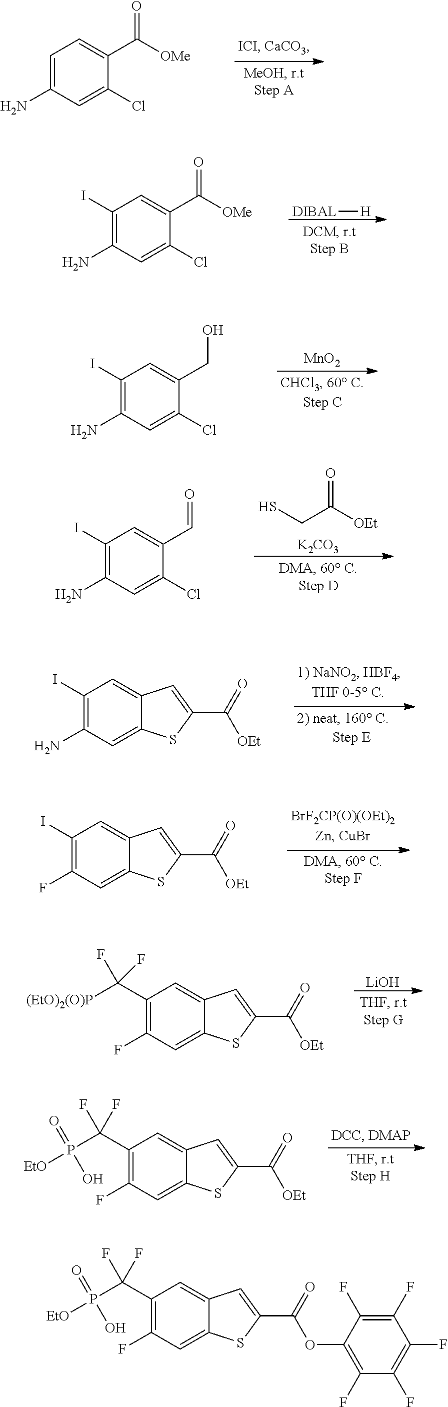

### Step A: Methyl 4-amino-2-chloro-5-iodobenzoate

To a suspension of methyl 4-amino-2-chlorobenzoate (5.0 g, 26.9 mmol, 1 eq.) and CaCO₃ (5.4 g, 53.8 mmol, 2.0 eq.) in MeOH (54 mL) was added a solution of ICl (4.8 g, 29.5 mmol, 1.1 eq.) in DCM (25 mL) at room temperature. The reaction mixture was stirred at room temperature for 16 hrs. After completion, the reaction mixture was filtered through a pad of Celite®. The filtrate was concentrated under reduced pressure to give a residue. The residue was triturated from petroleum ether: ethyl acetate=3:1 (30 mL) to give methyl 4-amino-2-chloro-5-iodobenzoate (6.1 g, 19.5 mmol, 73%) as a yellow solid. LCMS (ESI) m/z=312 [M+H]⁺. ¹H NMR (400 MHz, DMSO-d₆) δ 8.10 (s, 1H), 6.80 (s, 1H), 6.24 (s, 2H), 3.75 (s, 3H).

### Step B: (4-amino-2-chloro-5-iodophenyl)methanol

To a solution of methyl 4-amino-2-chloro-5-iodobenzoate (6.1 g, 19.5 mmol, 1 eq.) in DCM (120 mL) and THF (30 mL) at −78° C. under nitrogen was slowly added DIBAL-H (48.7 mL, 48.7 mmol, 2.5 eq.) over 30 minutes. After addition, the reaction mixture was stirred at room temperature for 30 min. After completion, the reaction mixture was quenched with saturated potassium sodium tartrate solution (100 mL) at 0° C. and stirred at room temperature for 2 hrs. The mixture was extracted with DCM (200 mL). The organic phase was washed with brine (50 mL), dried over anhydrous Na₂SO₄, filtered and concentrated under reduced pressure to afford (4-amino-2-chloro-5-iodophenyl)methanol (5.0 g, 17.6 mmol, 91%) as a yellow solid. LCMS (ESI) m/z=284 [M+H]⁺.

### Step C: 4-amino-2-chloro-5-iodobenzaldehyde

To a solution of (4-amino-2-chloro-5-iodophenyl)methanol (4.0 g, 14.1 mmol, 1 eq.) in CHCl₃ (50 mL) was added MnO₂ (7.4 g, 84.6 mmol, 6.0 eq.) at room temperature. After addition, the mixture was stirred at 60° C. for 5 hrs. After completion, the reaction mixture was cooled to room temperature and filtered. The filtrate was concentrated under reduced pressure to give 4-amino-2-chloro-5-iodobenzaldehyde (3.80 g, 13.5 mmol, 96%) as a yellow solid. LCMS: m/z=282[M+H]⁺.

### Step D: ethyl 6-amino-5-iodobenzo[b]thiophene-2-carboxylate

To a solution of 4-amino-2-chloro-5-iodobenzaldehyde (900 mg, 3.19 mmol, 1 eq.) and K₂CO₃ (881 mg, 6.38 mmol, 2.0 eq.) in DMA (10 mL) was added ethyl 2-sulfanylacetate (420 mg, 3.50 mmol, 1.1 eq.) at room temperature. After addition, the reaction mixture was stirred at 60° C. for 16 hrs. The reaction mixture was cooled to room temperature and poured into water (50 mL). The mixture was extracted with EtOAc (50 mL×3). The combined organic phases were washed with brine (50 mL), dried over anhydrous Na₂SO₄, filtered and concentrated under reduced pressure to give a residue. The residue was purified by flash column chromatography on silica gel to give ethyl 6-amino-5-iodo-1-benzothiophene-2-carboxylate (5,410 mg, 1.18 mmol, 37%) as a yellow solid. LCMS (ESI): m/z=348 [M+H]⁺.

### Step E: Ethyl 6-fluoro-5-iodobenzo[b]thiophene-2-carboxylate

To a solution of ethyl 6-amino-5-iodo-1-benzothiophene-2-carboxylate (410 mg, 1.18 mmol, 1 eq.) in THF (4 mL) at 0° C. was added 48% HBF₄ (2.4 g, 12.9 mmol, 11.0 eq.). A solution of NaNO₂ (73 mg, 1.06 mmol, 0.9 eq.) in water (4 mL) was then added dropwise and the resulting yellow suspension was stirred at 0° C. for 1 hr. The solid was collected by vacuum filtration and dried to give 400 mg of the diazonium salt. Diazobenzene tetrafluoroborate salt was heated to 160° C. for 3 hrs under nitrogen. The remaining material was purified by flash column chromatography on silica gel to give ethyl 6-fluoro-5-iodobenzo[b]thiophene-2-carboxylate (210 mg, 599 μmol, 51%) as a yellow oil. ¹H NMR (400 MHz, CDCl₃) δ 8.26 (d, J=5.8 Hz, 1H), 7.93 (s, 1H), 7.55 (d, J=7.6 Hz, 1H), 4.41 (q, J=7.1 Hz, 2H), 1.41 (t, J=7.1 Hz, 3H).

### Step F: Ethyl 5-((diethoxyphosphoryl)difluoromethyl)-6-fluorobenzo[b]thiophene-2-carboxylate

A mixture of diethyl (bromodifluoromethyl)phosphonate (317 mg, 1.19 mmol, 2.0 eq.) and activated Zn powder (67 mg, 1.19 mmol, 2.0 eq.) in DMA (4 mL) was stirred at 60° C. for 1 hr. Then, CuBr (170 mg, 1.19 mmol, 2.0 eq.) was added. The mixture was stirred 50° C. for 1 hr. A solution of ethyl 6-fluoro-5-iodobenzo[b]thiophene-2-carboxylate (210 mg, 599 μmol, 1 eq.) in DMA (2 mL) was added. After addition, the reaction mixture was stirred at 60° C. overnight under nitrogen. The reaction mixture was cooled to room temperature and filtered. The filtrate was diluted with water (10 mL), extracted with EtOAc (20 mL×3). The combined organic phases were washed with brine (20 mL), dried over Na₂SO₄, filtered and concentrated under reduced pressure to give a residue. The residue was purified by flash column chromatography on silica gel to give ethyl 5-[(diethoxyphosphoryl)difluoromethyl]-6-fluoro-1-benzothiophene-2-carboxylate (94.0 mg, 229 μmol, 38%) as a yellow oil. LCMS (ESI): m/z=411 [M+H]⁺.

### Step G: 5-((ethoxy(hydroxy)phosphoryl)difluoromethyl)-6-fluorobenzo[b]thiophene-2-carboxylic Acid

To a solution of 5-[(diethoxyphosphoryl)difluoromethyl]-6-fluoro-1-benzothiophene-2-carboxylate (7, 94 mg, 229 μmol, 1 eq.) in a mixture of THF (2 mL) and H₂O (0.5 mL) was added LiOH. H₂O (48 mg, 1.14 mmol, 5.0 eq.) at room temperature. After addition, the reaction mixture was stirred room temperature overnight. After completion, the reaction mixture was neutralized carefully with 5 N aqueous HCl solution until the pH was adjusted to pH=3. The mixture was extracted with EtOAc (10 mL×2). The organic phase was washed with brine (10 mL), dried over Na₂SO₄, filtered concentrated under reduced pressure to give 5-{[ethoxy(hydroxy)phosphoryl]difluoromethyl}-6-fluoro-1-benzothiophene-2-carboxylic acid (50.0 mg, 141 μmol, 62%) as a yellow solid. LCMS (ESI): m/z=355 [M+H]⁺.

### Step H: Perfluorophenyl 5-((ethoxy(hydroxy)phosphoryl)difluoromethyl)-6-fluorobenzo[b]thiophene-2-carboxylate

To a solution of 5-{[ethoxy(hydroxy)phosphoryl]difluoromethyl}-6-fluoro-1-benzothiophene-2-carboxylic acid (50 mg, 141 μmol, 1 eq.), pentafluorophenol (26 mg, 141 μmol, 1 eq.) and DMAP (2 mg, 14.1 μmol, 0.1 eq.) in THF (10 mL) was added DCC (41 mg, 197 μmol, 1.4 eq.) at 0° C. After addition, the reaction mixture was stirred at room temperature overnight. After completion, the reaction mixture was diluted with water (10 mL), extracted with EtOAc (10 mL×3). The organic phase was washed with brine (10 mL), dried over anhydrous Na₂SO₄, filtered and concentrated under reduced pressure to give a residue. The residue was purified by flash column chromatography on silica gel to give perfluorophenyl 5-((ethoxy(hydroxy)phosphoryl)difluoromethyl)-6-fluorobenzo[b]thiophene-2-carboxylate (60.0 mg, 115 μmol, 82%) as a white solid. LCMS (ESI): m/z=520 [M+H]⁺.

Bis-SATE and Mono-SATE of diFs Naphthyl PFP Ester:

### Preparation of Perfluorophenyl 7-((bis(2-(butyrylthio)ethoxy)phosphoryl)difluoromethyl)-2-naphthoate and Perfluorophenyl 7-(((2-(butyrylthio)ethoxy)(hydroxy)phosphoryl)difluoromethyl)-2-naphthoate

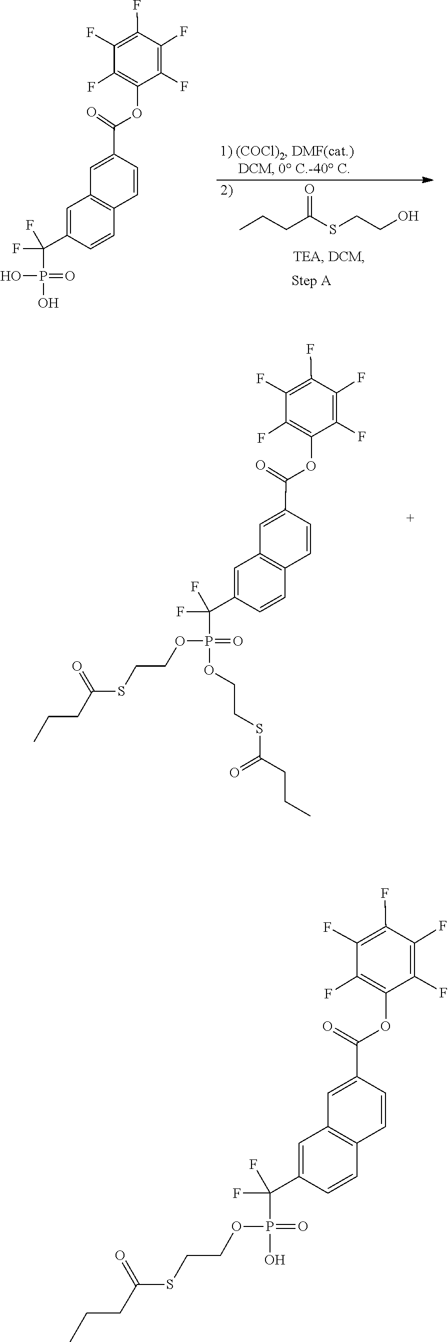

### Step A: Perfluorophenyl 7-((bis(2-(butyrylthio)ethoxy)phosphoryl)difluoromethyl)-2-naphthoate and Perfluorophenyl 7-(((2-(butyrylthio)ethoxy)(hydroxy)phosphoryl)difluoromethyl)-2-naphthoate

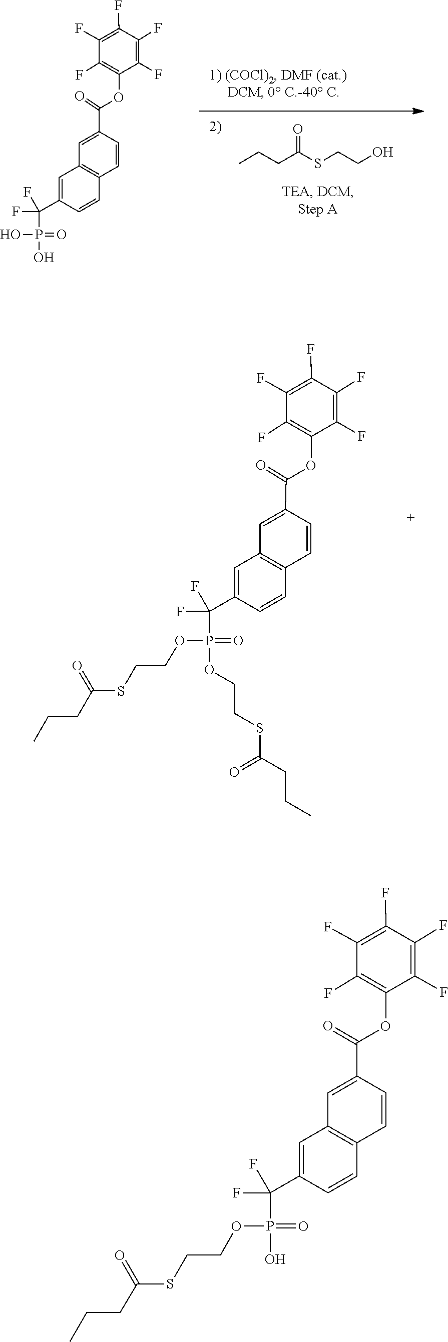

To a solution of (difluoro(7-((perfluorophenoxy)carbonyl)naphthalen-2-yl)methyl)phosphonic acid (1 g, 2.10 mmol, 1 eq.) in dry DCM (18 mL) was added oxalyl chloride (266 mg, 2.13 mmol, 1.0 eq.) dropwise followed with DMF (155 mg, 2.13 mmol, 1.0 eq.) at 25° C. The reaction mixture was warmed to 40° C., then stirred for an additional 1.2 hrs under reflux. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (14 mL) then added to a mixture of S-(2-hydroxyethyl) butanethioate (641 mg, 4.33 mmol, 2.2 eq.) and triethylamine (198 mg, 1.97 mmol, 1.0 eq.) in anhydrous DCM (15 mL) at 0° C. The reaction was allowed to warm to room temperature, and stirred for an additional 12 hrs. The reaction progress was monitored by LCMS, and after completion, the reaction was quenched by adding H₂O (10 mL) and concentrated under reduced pressure. The residue was purified by C18 column chromatography to afford perfluorophenyl 7-((bis(2-(butyrylthio)ethoxy)phosphoryl)difluoromethyl)-2-naphthoate (2A, 680 mg, 1.13 mmol, 58%) as a white solid and perfluorophenyl 7-(((2-(butyrylthio)ethoxy)(hydroxy)phosphoryl)difluoromethyl)-2-naphthoate (2B, 65.0 mg, 89.2 μmol) as a white solid. 2A: LCMS (ESI): m/z=729 [M+H]+ 0.2B: LCMS (ESI): m/z=599 [M+H]⁺.

### Step B: Perfluorophenyl 7-(((2-(butyrylthio)ethoxy)(((isopropoxycarbonyl)oxy)methoxy)phosphoryl)difluoromethyl)-2-naphthoate

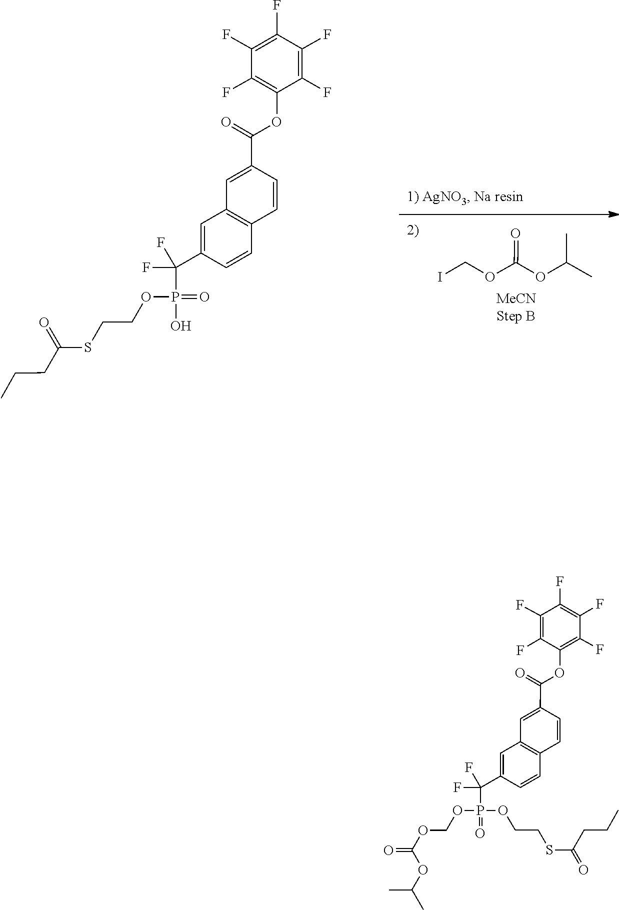

To a solution of perfluorophenyl 7-(((2-(butyrylthio)ethoxy)(hydroxy)phosphoryl)difluoromethyl)-2-naphthoate (300 mg, 501 μmol, 1 eq.) in a mixture of deionized H₂O (8 mL) and THF (2 mL) was added Amberlite IR120® resin (Na⁺ form) (233 mg, 751 μmol, 1.5 eq.). The resulting mixture was stirred at room temperature for 1 hr, then the excess of the resin was removed by filtration. AgNO₃ (127 mg, 751 μmol, 1.5 eq.) in deionized H₂O (2 mL) was then added to the resulting solution. After addition, the resulting mixture was stirred at room temperature for an additional 1 hr. During this period, the silver salt formed as a white precipitate, which was collected via filtration. The filter cake was washed with cool H₂O (2 mL×3), and the silver salt was further dried under reduced pressure to get a dry powder, which was pure enough for next step without further purification.

The isolated mono-Ag salt was suspended in ACN (8 mL), and iodomethyl isopropyl carbonate (244 mg, 1.00 mmol, 2 eq.) was added dropwise. After addition, the resulting mixture was stirred at 40° C. for an additional 12 hrs. The reaction progress was monitored by LCMS, and after completion, the unreacted silver salt was recovered by filtration, and the filtrate was concentrated under reduced pressure. The resulting residue was purified by prep-TLC to give perfluorophenyl 7-(((2-(butyrylthio)ethoxy)(((isopropoxycarbonyl)oxy)methoxy)phosphoryl)difluoromethyl)-2-naphthoate (30 mg, 41.9 μmol, 8%) as colorless oil. LCMS (ESI) m/z=737[M+Na]⁺.

### Preparation of 5-((diethoxyphosphoryl)difluoromethyl)thieno[2,3-b]pyridine-2-carboxylic Acid

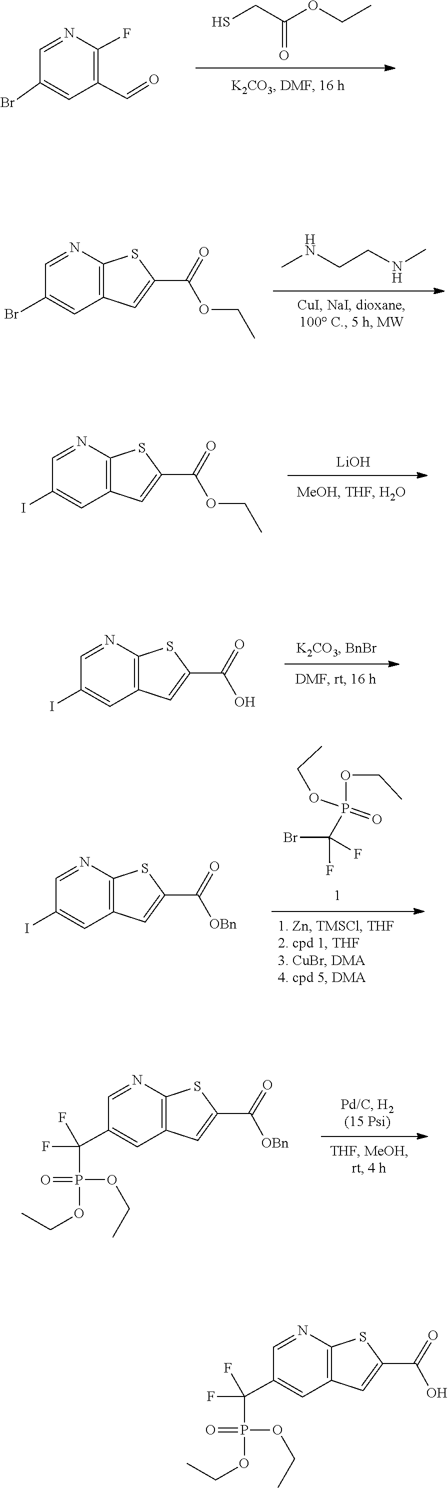

### Step 1: Ethyl 5-bromothieno[2,3-b]pyridine-2-carboxylate

To a solution of 5-bromo-2-fluoropyridine-3-carbaldehyde (10 g, 49 mmol, 1 eq) in dimethylformamide (70 mL) was added potassium carbonate (27 g, 0.2 mol, 4 eq) and ethyl 2-sulfanylacetate (6.5 g, 54 mmol, 1.1 eq). The mixture was stirred at 60° C. for 16 hours. The reaction was diluted with water (200 mL) and extracted with ethyl acetate (200 mL×2), the combined organic layers were washed with saturated brine (200 mL×2), dried over Na₂SO₄, filtered and concentrated under reduced pressure to give a residue. The residue was purified by flash silica gel chromatography to give ethyl 5-bromothieno[2,3-b]pyridine-2-carboxylate (13 g, 96% yield) as a yellow solid. LCMS: m/z (M+H)⁺=287.9

### Step 2: Ethyl 5-iodothieno[2,3-b]pyridine-2-carboxylate

To a solution of ethyl 5-bromothieno[2,3-b]pyridine-2-carboxylate (13 g, 45 mmol, 1 eq) and sodium iodide (14 g, 91 mmol, 2 eq) in dioxane (90 mL) was added N¹,N²-dimethylethane-1,2-diamine (0.4 g, 4.5 mmol, 0.1 eq) and CuI (0.4 g, 2.3 mmol, 0.05 eq), then the mixture was stirred at 100° C. for 5 hours under N₂ in microwave condition. The reaction mixture was concentrated under reduced pressure to give a residue. The residue was purified by flash silica gel chromatography to give ethyl 5-iodothieno[2,3-b]pyridine-2-carboxylate (13 g, 89% yield) as a yellow solid. LCMS: m/z (M+H)⁺=333.8.

### Step 3: 5-iodothieno[2,3-b]pyridine-2-carboxylic Acid

To a solution of ethyl 5-iodothieno[2,3-b]pyridine-2-carboxylate (12 g, 36 mmol, 1 eq) in H₂O:THF:MeOH=1:1:1 (90 mL) was added lithium hydroxide (2.6 g, 0.1 mol, 3 eq). The mixture was stirred at 25° C. for 4 hours. The reaction mixture was concentrated under reduced pressure to give a residue. Then the residue was diluted with water (50 mL) and extracted with ethyl acetate (50 mL). The aqueous phase was used FA (1 M) to adjust the PH=5-6, and extracted with ethyl acetate (50 mL×2), dried over Na₂SO₄, filtered and concentrated under reduced pressure to give crude 5-iodothieno[2,3-b]pyridine-2-carboxylic acid (8 g, crude) as a yellow oil. LCMS: m/z (M+H)⁺=305.9.

### Step 4: Benzyl 5-iodothieno[2,3-b]pyridine-2-carboxylate

To a solution of 5-iodothieno[2,3-b]pyridine-2-carboxylic acid (8 g, 26 mmol, 1 eq) in DMF (50 mL) was added potassium carbonate (7.2 g, 52 mmol, 2 eq) and (bromomethyl)benzene (4.9 g, 29 mmol, 1.1 eq). The mixture was stirred at 25° C. for 16 hours. The reaction mixture was diluted with water (100 mL) and extracted with ethyl acetate (100 mL×2), the combined organic layers were washed with saturated brine (100 mL×2), dried over Na₂SO₄, filtered and concentrated under reduced pressure to give a residue. The residue was purified by flash silica gel chromatography to give benzyl 5-iodothieno[2,3-b]pyridine-2-carboxylate (4.4 g, 43% yield) as a yellow solid. LCMS: m/z (M+H)⁺=396.1. ¹H NMR (400 MHz, DMSO-d₆) δ 9.03-8.63 (m, 1H), 8.12 (s, 1H), 7.62-7.08 (m, 6H), 5.55-5.02 (m, 2H).

### Step 5: Benzyl 5-((diethoxyphosphoryl)difluoromethyl)thieno[2,3-b]pyridine-2-carboxylate

To a suspension of zinc (0.4 g, 5.7 mmol, 2 eq) in degassed, anhydrous THF (10 mL) was added chlorotrimethylsilane (0.1 g, 1.2 mmol, 0.4 eq) and the suspension was heated to 50° C. for 1.5 hours with vigorous stirring. The solution of diethyl (bromodifluoromethyl)phosphonate (1.6 g, 6.1 mmol, 2 eq) in THF (15 mL) was added dropwise (temperature kept between 50-55° C.). The reaction mixture was stirred at 50° C. for 2 hours and lambda1-copper(1+) bromide (0.4 g, 3.0 mmol, 1.0 eq) and dimethylacetamide (6.7 mL, 73 mmol, 24 eq) were added and the mixture was stirred at rt for 30 min. A solution of benzyl 5-iodothieno[2,3-b]pyridine-2-carboxylate (1.2 g, 3.0 mmol, 1 eq) in N,N-dimethylacetamide (10 mL) was slowly added to the reaction mixture, then was heated to 50° C. for 12 hours. The reaction mixture was diluted with water (50 mL) and extracted with ethyl acetate (50 mL×2), the combined organic layers were washed with saturated brine (50 mL×2), dried over Na₂SO₄, filtered and concentrated under reduced pressure to give a residue. The residue was purified by reversed phase (Column: 120 g Flash Column Welch Ultimate XB_C18 20-40 μm; 120 A, Mobile phase: H₂O+ACN; Gradient B %: 10-65% 15 min; 65% 3 min) to lyophilized to give benzyl 5-((diethoxyphosphoryl)difluoromethyl)thieno[2,3-b]pyridine-2-carboxylate (0.6 g, 42% yield) as a yellow oil. LCMS: m/z (M+H)⁺=456.3. ¹H NMR (400 MHz, METHANOL-d₄) δ 8.84 (s, 1H), 8.59 (s, 1H), 8.24 (s, 1H), 7.57-7.31 (m, 5H), 5.43 (s, 2H), 4.38-4.16 (m, 4H), 1.33 (t, J=7.2 Hz, 6H)

### Step 6: 5-((diethoxyphosphoryl)difluoromethyl)thieno[2,3-b]pyridine-2-carboxylic Acid

To a solution of benzyl 5-((diethoxyphosphoryl)difluoromethyl)thieno[2,3-b]pyridine-2-carboxylate (0.3 g, 0.7 mmol, 1 eq) in methanol (1.5 mL) and tetrahydrofuran (1.5 mL) was added dry Pd/C (30 mg) under N₂. The mixture was stirred at 25° C. under H₂ (15 psi) for 4 hours. The reaction mixture was filtered and the filter was concentrated to give crude 5-((diethoxyphosphoryl)difluoromethyl)thieno[2,3-b]pyridine-2-carboxylic acid (0.2 g, crude) as a yellow oil. LCMS: m/z (M+H)⁺=365.9.

### Preparation of 6-(difluoro(phosphono)methyl)-2-naphthoic Acid

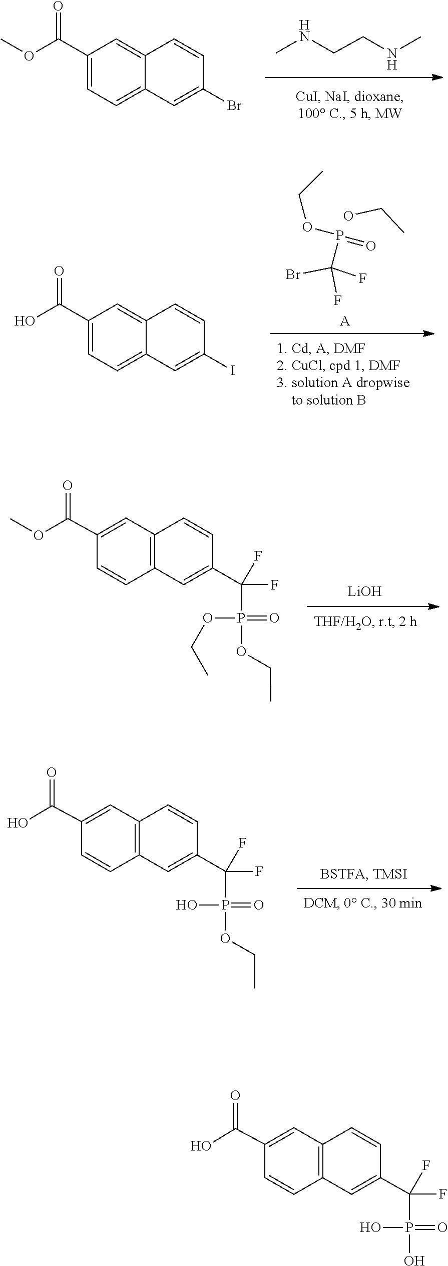

### Step 1: Methyl 6-iodo-2-naphthoate

To a solution of methyl 6-bromonaphthalene-2-carboxylate (5.0 g, 19 mmol, 1 eq) and sodium iodide (5.6 g, 38 mmol, 2 eq) in dioxane (50 mL) was added N¹,N²-dimethylethane-1,2-diamine (0.17 g, 1.9 mmol, 0.1 eq) and CuI (0.18 g, 0.94 mmol, 0.05 eq), then the mixture was stirred at 100° C. for 5 hours under N₂ in microwave condition. The reaction mixture was diluted with water (50 mL) and extracted with ethyl acetate (50 mL×2), the combined organic layers were washed with saturated brine (50 mL×2), dried over Na₂SO₄, filtered and concentrated under reduced pressure to give a residue. The residue was purified by RP-flash (column: 330 g Flash Column, Welch Ultimate XB_C18 20-40 μm; 120 A; mobile phase: 5-80% 35 min; 80% 10 min) to lyophilized to give methyl 6-iodo-2-naphthoate (4.2 g, 70% yield) as a white solid. LCMS: m/z (M+H)⁺=313.0

### Step 2: Methyl 6-((diethoxyphosphoryl)difluoromethyl)-2-naphthoate

To a solution of diethyl (bromodifluoromethyl)phosphonate (5.4 g, 20 mmol, 3.5 eq) in DMF (50 mL) was added cadmium (2.3 g, 21 mmol, 3.6 eq) and stirred at 25° C. for 8 hours under N₂ to give solution A. The solution of methyl 6-iodo-2-naphthoate (1.8 g, 5.8 mmol, 1 eq) in dimethylformamide (20 mL) was added CuCl (1.1 g, 11 mmol, 2.0 eq) and stirred at 25° C. for 2 hours under N₂ to give solution B. Then the solution A was filtered and the filtrate was added to the solution B, and the mixture was stirred at 25° C. for 16 hours under N₂. The reaction mixture was filtered and filtrate was quenched by water 100 mL, and extracted with ethyl acetate 240 mL (80 mL×3). The combined organic layers were washed with brine 240 mL (80 mL×3), dried over Na₂SO₄, filtered and concentrated under reduced pressure to give a residue. The residue was purified by RP-flash (column: 330 g Flash Column, Welch Ultimate XB_C18 20-40 μm; 120 A; mobile phase: 5-60% 30 min; 60% 5 min) to give methyl 6-((diethoxyphosphoryl) difluoromethyl)-2-naphthoate (1.0 g, 47% yield) as a white solid. LCMS: m/z (M+1)⁺=373.0

### Step 3: 6-((ethoxy(hydroxy)phosphoryl)difluoromethyl)-2-naphthoic Acid

To a solution of methyl 6-((diethoxyphosphoryl) difluoromethyl)-2-naphthoate (0.35 g, 0.94 mmol, 1 eq) in tetrahydrofuran (3 mL) and water (1 mL) was added lithium(1+) hydrate hydroxide (79 mg, 1.9 mmol, 2 eq), then the mixture was stirred at 25° C. for 2 hours.

The reaction mixture was concentrated under reduced pressure to give 6-((ethoxy(hydroxy)phosphoryl)difluoromethyl)-2-naphthoic acid (0.29 g, crude) as a white solid. LCMS: m/z (M+H)⁺=331.0. ¹H NMR (400 MHz, DMSO-d₆) δ 8.38 (s, 1H), 8.05-8.00 (m, 2H), 7.91 (d, J=8.8 Hz, 1H), 7.84 (d, J=8.4 Hz, 1H), 7.64 (d, J=8.8 Hz, 1H), 3.80-3.76 (m, 2H), 1.07 (t, J=7.2 Hz, 3H)

### Step 4: 6-(difluoro(phosphono)methyl)-2-naphthoic Acid

To a solution of 6-((ethoxy(hydroxy)phosphoryl)difluoromethyl)-2-naphthoic acid (0.10 g, 0.30 mmol, 1 eq) in methylene chloride (1 mL) was added BSTFA (0.46 g, 1.8 mmol, 6 eq) at 0° C., then the mixture was stirred at 0° C. for 10 min, then TMSI (0.24 g, 1.2 mmol, 4 eq) in methylene chloride (1 mL) was added at 0° C. and stirred at 0° C. for 20 min. The reaction was concentrated under reduced pressure to give a residue. The residue was purified by prep-HPLC (column: Phenomenex Luna C18 150×25×10 um; mobile phase: [water (0.225% FA)-ACN]; B %: 10%-40%, 11 min) to give 6-(difluoro(phosphono) methyl)-2-naphthoic acid (20 mg, 25% yield) as a yellow oil. LCMS: m/z (M+H)⁺=302.9

### Preparation of 6-(dimethoxyphosphoryl)-2-naphthoic Acid

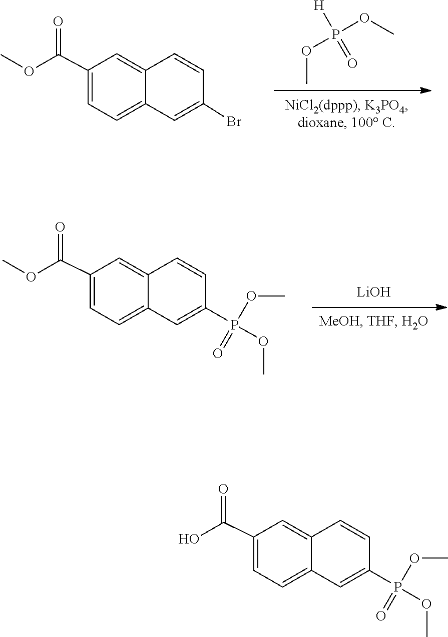

### Step 1: Methyl 6-(dimethoxyphosphoryl)-2-naphthoate

To a solution of methyl 6-bromonaphthalene-2-carboxylate (5.0 g, 19 mmol, 1 eq) in dioxane (50 mL) was added K₃PO₄ (8.0 g, 38 mmol, 2 eq), NiCl₂(dppp) (1.0 g, 1.9 mmol, 0.1 eq) was added under N₂, then the solution of dimethyl phosphonate (4.1 g, 38 mmol, 2 eq) in dioxane (50 mL) was dropwise. The reaction mixture was stirred at 100° C. for 12 hours. The reaction mixture was diluted with water (200 mL) and extracted with ethyl acetate (200 mL×2), the combined organic layers were washed with saturated brine (200 mL×2), dried over Na₂SO₄, filtered and concentrated under reduced pressure to give a residue. The residue was purified by column chromatography to give methyl 6-(dimethoxyphosphoryl)-2-naphthoate (1.5 g, 27% yield) as a white solid. LCMS: m/z (M+H)⁺=295.3. ¹H NMR (400 MHz, CD₃OD) δ 8.66 (s, 1H), 8.45 (d, J=15.6 Hz, 1H), 8.19-8.08 (m, 3H), 7.81 (m, 1H), 3.98 (s, 3H), 3.85-3.76 (m, 6H)

### Step 2: 6-(dimethoxyphosphoryl)-2-naphthoic Acid

To a solution of methyl 6-(dimethoxyphosphoryl)-2-naphthoate (0.50 g, 1.7 mmol, 1 eq) in methanol (13 mL), tetrahydrofuran (13 mL) and water (13 mL) was added lithium(1+) hydrate hydroxide (0.14 g, 3.4 mmol, 2 eq). The mixture was stirred at 25° C. for 2 hours. The reaction mixture was diluted with water (50 mL) and extracted with ethyl acetate (50 mL×2), the aqueous was adjusted to pH=4-6 with the addition of HCl (1 M). Then the solution was extracted with ethyl acetate (50 mL×2), dried over Na₂SO₄, filtered and concentrated under reduced pressure to give crude 6-(dimethoxyphosphoryl)-2-naphthoic acid (0.30 g, crude) as a white solid.

### Preparation of 7-(dimethoxyphosphoryl)-2-naphthoic Acid

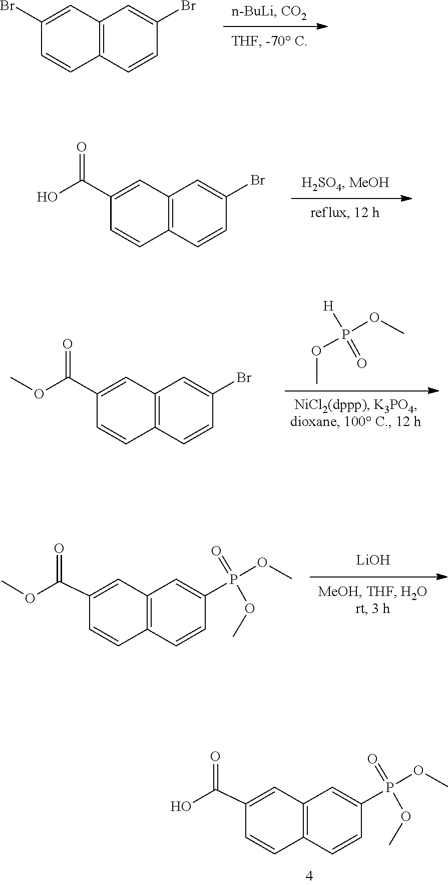

### Step 1: 7-bromo-2-naphthoic Acid

A solution of 2,7-dibromonaphthalene (20 g, 69 mmol, 1 eq) in tetrahydrofuran (200 mL) was cooled to −76° C. and n-BuLi (4.5 g, 70 mmol, 1 eq) was dropwise. Then CO₂ (gas) was bubbled through the reaction mixture. After 15 minutes the cold bath was removed and the reaction was warmed to room temperature with continued CO₂ bubbling and the reaction mixture was stirred at room temperature for 1.5 hours. The reaction mixture was quenched with saturated NH₄Cl (200 mL) and extracted with ethyl acetate (200 mL×2), the combined organic layers were washed with saturated brine (200 mL×2), dried over Na₂SO₄, filtered and concentrated under reduced pressure to give 7-bromo-2-naphthoic acid (14 g, crude), obtained as a yellow solid. LCMS: m/z (M−H)⁻=250.0. ¹H NMR (400 MHz, DMSO-d₆) δ 8.60 (d, J=0.8 Hz, 1H), 8.43 (d, J=2.0 Hz, 1H), 8.07-8.01 (m, 2H), 8.01-7.95 (m, 1H), 7.77 (dd, J=2.2, 8.7 Hz, 1H)

### Step 2: methyl 7-bromo-2-naphthoate

A solution of 7-bromo-2-naphthoic acid (4.3 g, 17 mmol, 1 eq) in methanol (40 mL) and H₂SO₄ (8 mL) was stirred at 70° C. for 12 hours. The reaction mixture was concentrated under reduced pressure to give a residue. The residue was diluted with water (50 mL) and extracted with ethyl acetate (50 mL×2), the combined organic layers were washed with saturated brine (50 mL×2), dried over Na₂SO₄, filtered and concentrated under reduced pressure to give a residue. The residue was purified by column chromatography to give methyl 7-bromo-2-naphthoate (3.6 g, 84% yield) as a yellow solid. ¹H NMR (400 MHz, METHANOL-d₄) δ 8.58-8.53 (m, 1H), 8.22 (d, J=2.0 Hz, 1H), 8.08-8.03 (m, 1H), 7.95 (d, J=8.4 Hz, 1H), 7.87 (d, J=8.4 Hz, 1H), 7.73-7.67 (m, 1H), 3.97 (s, 3H)

### Step 3: Methyl 7-(dimethoxyphosphoryl)-2-naphthoate

To a solution of methyl 7-bromo-2-naphthoate (3.3 g, 12 mmol, 1 eq) in dioxane (30 mL) was added tripotassium phosphate (5.3 g, 24 mmol, 2 eq) and dimethyl phosphonate (2.7 g, 25 mmol, 2 eq), then NiCl₂(dppp) (0.67 g, 1.2 mmol, 0.1 eq) was added under N₂, the mixture was stirred at 100° C. for 12 hours. The reaction mixture was diluted with water (100 mL) and extracted with ethyl acetate (100 mL×2), the combined organic layers were washed with saturated brine (100 mL×2), dried over Na₂SO₄, filtered and concentrated under reduced pressure to give a residue. The residue was purified by column chromatography to give methyl 7-(dimethoxyphosphoryl)-2-naphthoate (0.60 g, 16% yield) as a white solid. LCMS: m/z (M+H)⁺=295.1

### Step 4: 7-(dimethoxyphosphoryl)-2-naphthoic Acid

To a solution of methyl 7-(dimethoxyphosphoryl)-2-naphthoate (0.6 g, 2.0 mmol, 1 eq) in water (2 mL), tetrahydrofuran (2 mL) and methanol (2 mL) was added lithium hydroxide hydrate (85 mg, 2.0 mmol, 1 eq), and the mixture was stirred at 25° C. for 3 hours. The reaction mixture was concentrated under reduced pressure to remove the THF and methanol to give a suspension. The suspension was purified by prep-HPLC (column: Phenomenex Luna C18 150×25 mm×10 um, mobile phase: water (0.225% FA)-ACN; B %: 13%-43%, 11.5 min) to lyophilization to give 7-(dimethoxyphosphoryl)-2-naphthoic acid (0.45 g, 79% yield) as a white solid. ¹H NMR (400 MHz, METHANOL-d₄) δ 8.77 (s, 1H), 8.55 (d, J=15.6 Hz, 1H), 8.27-8.18 (m, 1H), 8.16-8.10 (m, 1H), 8.09-8.04 (m, 1H), 7.93-7.82 (m, 1H), 3.85 (d, J=11.2 Hz, 6H)

### Preparation of ((2-(((5S,8S,10aR)-3-acetyl-8-(methyl(phenyl)carbamoyl)-6-oxodecahydropyrrolo[1,2-a][1,5]diazocin-5-yl)carbamoyl)benzo[d]thiazol-5-yl)difluoromethyl)phosphonic Acid

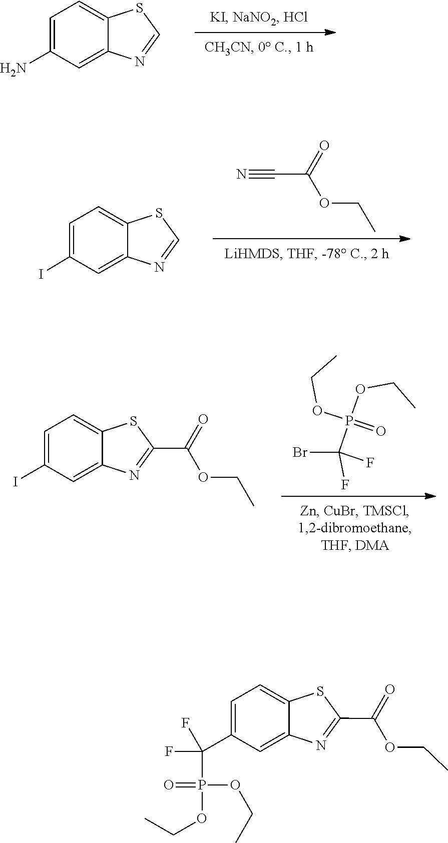

### Step 1: 5-iodobenzo[d]thiazole

To a solution of benzo[d]thiazol-5-amine (10 g, 67 mmol, 1 eq) in acetonitrile (50 mL) was added HCl (7.3 g, 200 mmol, 3 eq) at 0° C., followed by the addition of sodium nitrite (5.5 g, 80 mmol, 1.2 eq) in water (50 mL). The solution was stirred at 0° C. for 30 min. Then potassium iodide (17 g, 0.1 mol, 1.5 eq) in water (50 mL) was added at 0° C. The reaction mixture was stirred at 0° C. for 1 hour to give a black suspension. The reaction mixture was poured into water (100 mL). The aqueous phase was extracted with ethyl acetate (200 mL×2). The combined organic phase was washed with brine (200 mL×2), dried with anhydrous Na₂SO₄, filtered and concentrated under vacuum to give a residue. The residue was purified by silica gel chromatography (column height: 250 mm, diameter: 100 mm, 100-200 mesh silica gel, Petroleum ether/Ethyl acetate=100/1, 5/1) to give 5-iodobenzo[d]thiazole (9.0 g, 34 mmol, 52% yield) as a yellow solid. LCMS: m/z (M+H)⁺=243.9

### Step 2: Ethyl 5-iodobenzo[d]thiazole-2-carboxylate

To a solution of 5-iodobenzo[d]thiazole (1 g, 3.8 mmol, 1 eq) and ethyl cyanoformate (0.76 g, 7.7 mmol, 2 eq) in THF (15 mL) at −78° C. under N₂, then lithium bis(trimethylsilyl)azanide (1.6 g, 9.6 mmol, 2.5 eq) was added at −78° C. and the reaction mixture was stirred at −78° C. for 1 hour to give a yellow solution. The reaction solution was poured into water (45 mL). The aqueous phase was extracted with ethyl acetate (45 mL×2). The combined organic phase was washed with brine (45 mL×2), dried with anhydrous Na₂SO₄, filtered and concentrated in vacuum to give a residue. The residue was purified by silica gel chromatography (column height: 250 mm, diameter: 100 mm, 100-200 mesh silica gel) to give ethyl 5-iodobenzo[d]thiazole-2-carboxylate (1 g, 3.0 mmol, 79% yield) as a yellow oil. LCMS: m/z (M-28+H)+=305.8. ¹H NMR (400 MHz, CHLOROFORM-d) δ 8.97 (s, 1H), 8.52 (d, J=0.8 Hz, 1H), 7.73 (d, J=2.0 Hz, 1H), 4.57 (d, J=7.2 Hz, 2H), 1.50 (t, J=7.2 Hz, 3H).

### Step 3: Ethyl 5-((diethoxyphosphoryl)difluoromethyl)benzo[d]thiazole-2-carboxylate

To a suspension of zinc (0.22 g, 3.4 mmol, 2.5 eq) in THF (5 mL) was added chlorotrimethylsilane (59 mg, 0.54 mmol, 0.4 eq) under N₂, then the reaction mixture was stirred at 55° C. for 1.5 hour. The contents were cooled to 30° C. and a solution of diethyl (bromodifluoromethyl)phosphonate (0.72 g, 2.7 mmol, 2 eq) in THF (5 mL) was added dropwise (temperature kept between 50-55° C.). The reaction mixture was stirred at 30° C. for 2 hours (the clouded suspension became solution) and copper(1⁺) bromide (0.19 g, 1.4 mmol, 1.0 eq) in DMA (2 mL) were added and the mixture was stirred at 20° C. for 10 min. A solution of ethyl 5-iodobenzo[d]thiazole-2-carboxylate (0.45 g, 1.4 mmol, 1 eq) in DMA (2 mL) was added and the reaction mixture was stirred at 55° C. for 16 hours to give a green suspension. The reaction solution was poured into ice-water (w/w=1/1) (35 mL). The aqueous phase was extracted with ethyl acetate (35 mL×2). The combined organic phase was washed with brine (35 mL×2), dried with anhydrous Na₂SO₄, filtered and concentrated in vacuum to give a residue. The residue was purified by silica gel chromatography to give ethyl 5-((diethoxyphosphoryl) difluoromethyl)benzo[d]thiazole-2-carboxylate (0.1 g, 0.25 mmol, 19% yield) as a yellow solid. LCMS: m/z (M+H)⁺=394.4. ¹H NMR (400 MHz, CHLOROFORM-d) δ 8.49 (s, 1H), 8.08 (d, J=8.8 Hz, 1H), 7.83 (d, J=8.8 Hz, 1H), 4.58 (q, J=7.2 Hz, 2H), 4.30-4.19 (m, 4H), 1.51 (t, J=7.2 Hz, 3H), 1.34 (t, J=7.2 Hz, 6H).

### Preparation of ((2-(((5S,8S,10aR)-3-acetyl-8-(cinnolin-6-yl(methyl)carbamoyl)-6-oxodecahydropyrrolo[1,2-a][1,5]diazocin-5-yl)carbamoyl)quinolin-7-yl)difluoromethyl)phosphonic Acid

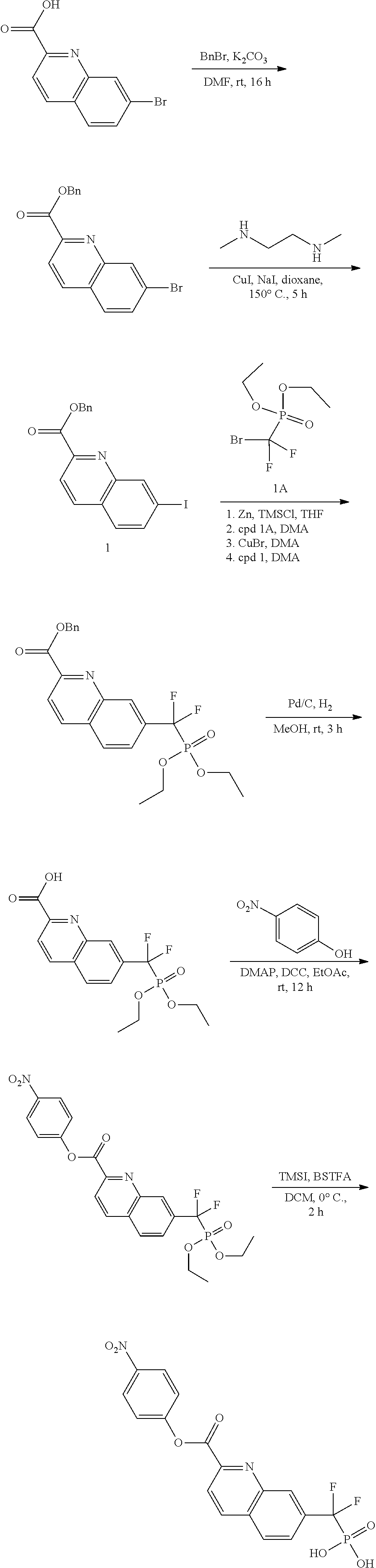

### Step 1: Benzyl 7-bromoquinoline-2-carboxylate

To a solution of 7-bromoquinoline-2-carboxylic acid (2.0 g, 7.9 mmol, 1 eq) in dimethylformamide (10 mL) was added (bromomethyl)benzene (1.3 g, 7.9 mmol, 1 eq) and potassium carbonate (2.2 g, 16 mmol, 2 eq). The mixture was stirred at 25° C. for 16 hours to give a white suspension. The mixture was diluted with water (30 mL) and extracted with ethyl acetate (30 mL×2), the combined organic layers were washed with saturated brine (30 mL×2), dried over Na₂SO₄, filtered and concentrated under reduced pressure to give a residue. The residue was purified by flash silica gel chromatography (40 g SepaFlash® Silica Flash Column, Eluent of 0-25% Petroleum ether/Ethyl acetate gradient @50 mL/min) to give benzyl 7-bromoquinoline-2-carboxylate (2.1 g, 77% yield) as a yellow solid. ¹H NMR (400 MHz, CHLOROFORM-d) δ 8.44 (s, 1H), 8.25-8.04 (m, 2H), 7.68-7.62 (m, 2H), 7.54-7.41 (m, 2H), 7.37-7.24 (m, 3H), 5.45 (s, 2H).

### Step 2: benzyl 7-iodoquinoline-2-carboxylate

To a solution of benzyl 7-bromoquinoline-2-carboxylate (2.0 g, 5.8 mmol, 1 eq) and sodium iodide (1.7 g, 12 mmol, 2 eq) in dioxane (10 mL) was methyl[2-(methylamino)ethyl]amine (51.4 mg, 584 μmol, 0.1 eq) and lambda1-copper(1+) iodide (56 mg, 0.29 mmol, 0.05 eq). The mixture was stirred at 150° C. for 5 hours under N₂ in microwave reactor to give a black suspection. The reaction mixture was concentrated under reduced pressure to give a residue. The residue was purified by flash silica gel chromatography (40 g SepaFlash® Silica Flash Column, Eluent of 0˜25% Petroleum ether/Ethyl acetate gradient @12 mL/min) was collected and concentrated in vacuo to give benzyl 7-iodoquinoline-2-carboxylate (1.3 g, 57% yield) as a white solid. LCMS: m/z (M+H)⁺=390.0. ¹H NMR (400 MHz, CHLOROFORM-d) δ 8.20-8.15 (m, 1H), 8.13-8.08 (m, 1H), 7.85-7.82 (M, 1H), 7.54-7.41 (m, 3H), 7.37-7.27 (m, 4H), 5.45 (s, 2H).

### Step 3: Benzyl 7-((diethoxyphosphoryl)difluoromethyl)quinoline-2-carboxylate

To a suspension of zinc (0.44 g, 6.7 mmol, 2 eq) in degassed, anhydrous tetrahydrofuran (30 mL) was added chlorotrimethylsilane (0.14 mg, 1.3 mmol, 0.4 eq) and the suspension was heated to 60° C. for 1.5 hours with vigorous stirring. The contents were cooled to 30° C. and a solution of diethyl (bromodifluoromethyl)phosphonate (1.1 g, 4.0 mmol, 1.2 eq) in tetrahydrofuran (10 mL) was added dropwise (temperature kept between 50-55° C.). The reaction mixture was stirred at 30° C. for 2 hours (the clouded suspension became solution) and copper (1⁺) bromide (479 mg, 3.34 mmol, 1.0 eq) and dimethylacetamide (7.0 g, 80 mmol, 24 eq) were added and the mixture was stirred at room temperature for 10 min. A solution of benzyl 7-iodoquinoline-2-carboxylate (1.3 g, 3.4 mmol, 1 eq) in tetrahydrofuran (10 mL) was slowly added to the reaction mixture, it was heated to 50° C. for 12 hours. The reaction mixture was diluted with water (50 mL) and extracted with ethyl acetate (50 mL×2), the combined organic layers were washed with saturated brine (50 mL×2), dried over Na₂SO₄, filtered and concentrated under reduced pressure to give a residue. The residue was purified by RP-flash (TFA system), freeze-drying to give benzyl 7-((diethoxyphosphoryl)difluoromethyl)quinoline-2-carboxylate (0.3 g, 19% yield) as a yellow oil. LCMS: m/z (M+H)⁺=450.1. ¹H NMR (400 MHz, CHLOROFORM-d) δ 8.59 (s, 1H), 8.43-8.33 (m, 1H), 8.32-8.22 (m, 1H), 8.04-7.97 (m, 1H), 7.96-7.89 (m, 1H), 7.61-7.53 (m, 2H), 7.47-7.33 (m, 3H), 5.57 (s, 2H), 4.39-4.15 (m, 4H), 1.35 (t, J=7.2, 6H).

### Step 4: Benzyl 7-iodoquinoline-2-carboxylate

To a solution of benzyl 7-((diethoxyphosphoryl)difluoromethyl)quinoline-2-carboxylate (0.32 mg, 0.71 mmol, 1 eq) in methanol (3 mL) was added wet Pd/C (0.1 g, 2.9 mmol) and degassed and purged with H₂ three times. The mixture was stirred at 25° C. for 3 hours under H₂ (15 psi) to give a black suspension. The reaction mixture was filtered and the filtrate was concentrated to give benzyl 7-((diethoxyphosphoryl) difluoromethyl)quinoline-2-carboxylate (0.25 g, crude). LCMS: m/z (M+H)⁺=360.1.

### Step 5: 4-nitrophenyl 7-((diethoxyphosphoryl)difluoromethyl)quinoline-2-carboxylate

To a solution of benzyl 7-((diethoxyphosphoryl)difluoromethyl)quinoline-2-carboxylate (50 mg, 0.14 mmol, 1 eq) in methylene chloride (1 mL) was added 4-dimethylaminopyridine (25 mg, 0.21 mmol, 1.5 eq), 4-nitrophenol (25 mg, 0.18 mmol, 1.3 eq) and dicyclohexylcarbodiimide (43 mg, 0.21 μmol, 1.5 eq). The mixture was stirred at 25° C. for 1 hour to give a yellow suspension. The reaction mixture was concentrated under reduced pressure to give a residue. The residue was purified by flash silica gel chromatography (4 g SepaFlash® Silica Flash Column, Eluent of 0-60% Petroleum ether/Ethyl acetate gradient @30 mL/min) to give 4-nitrophenyl 7-((diethoxyphosphoryl)difluoromethyl)quinoline-2-carboxylate (30 mg, 45% yield)., LCMS: m/z (M+H)⁺=481.1. ¹H NMR (400 MHz, CHLOROFORM-d) δ 8.65 (s, 1H), 8.49-8.45 (m, 1H), 8.42-8.36 (m, 2H), 8.16-8.11 (m, 1H), 8.08-8.03 (m, 1H), 8.00-7.94 (m, 1H), 7.59-7.47 (m, 1H), 6.97-6.84 (m, 1H), 4.40-4.05 (m, 4H), 1.42-1.31 (m, 6H).

### Step 6: (difluoro(2-((4-nitrophenoxy)carbonyl)quinolin-7-yl)methyl)phosphonic Acid

To a solution of 4-nitrophenyl 7-((diethoxyphosphoryl)difluoromethyl)quinoline-2-carboxylate (30 mg, 62 μmol, 1 eq) in methylene chloride (1 mL) was added trimethylsilyl (1E)-2,2,2-trifluoro-N-trimethylsilylethanimidate (96 mg, 0.37 mmol, 6 eq) and trimethylsilyl iodide (50 mg, 0.25 mmol, 4 eq). The mixture was stirred at 0° C. for 2 hours to give a yellow solution. The reaction mixture was concentrated under reduced pressure to give crude (difluoro(2-((4-nitrophenoxy)carbonyl)quinolin-7-yl)methyl)phosphonic acid (30 mg, crude). LCMS: m/z (M+H)⁺=425.1

### Preparation of ((6-(((5S,8S,10aR)-3-acetyl-8-(cinnolin-6-yl(methyl)carbamoyl)-6-oxodecahydropyrrolo[1,2-a][1,5]diazocin-5-yl)carbamoyl)isoquinolin-3-yl)difluoromethyl)phosphonic Acid

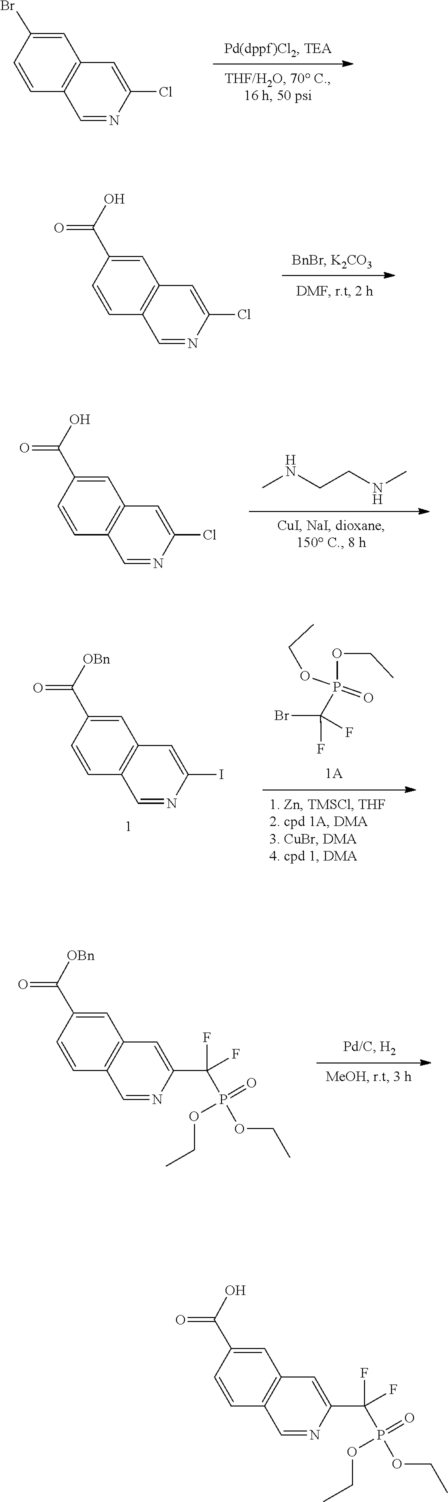

### Step 1: 3-chloroisoquinoline-6-carboxylic Acid

To a solution of 6-bromo-3-chloroisoquinoline (5.0 g, 21 mmol, 1 eq) in THF (45 mL) and H₂O (5 mL) was added iron(2⁺) bis((cyclopenta-2,4-diyn-1-yl)diphenylphosphane) palladium dichloride (1.5 g, 2.1 mmol, 0.1 eq) and triethylamine (4.2 g, 41 mmol, 2 eq). The mixture was stirred at 70° C. for 16 hours under CO (50 psi) to give a black solution. The reaction mixture was quenched by water (50 mL) and extracted with ethyl acetate 150 mL (50 mL×3), then the combined aqueous layer was collected. The combined aqueous layer was adjusted to pH=3-4 by 20 mL 1N formic acid, then extracted with ethyl acetate 150 mL (50 mL×3). The combined organic layer was washed with saturated brine 450 mL (150 mL×3), dried over Na₂SO₄, filtered and concentrated under reduced pressure to give 3-chloroisoquinoline-6-carboxylic acid (3.5 g, 76% yield) as a white solid. LCMS: m/z (M+H)⁺=208.1. ¹H NMR (400 MHz, DMSO-d₆) δ 13.94-13.02 (m, 1H), 9.33 (s, 1H), 8.64 (s, 1H), 8.33-8.22 (m, 2H), 8.13 (dd, J=1.6, 8.4 Hz, 1H).

### Step 2: Benzyl 3-chloroisoquinoline-6-carboxylate

To a solution of 3-chloroisoquinoline-6-carboxylic acid (3.5 g, 17 mmol, 1 eq) and (bromomethyl) benzene (4.3 g, 25 mmol, 1.5 eq) in DMF (40 mL) was added dipotassium carbonate (4.6 g, 34 mmol, 2 eq). The mixture was stirred at 25° C. for 2 hours to give a gray solution. The reaction mixture was quenched by water 50 mL and extracted with ethyl acetate 150 mL (50 mL×3). The combined organic layers were washed with brine (50 mL×3), dried over Na₂SO₄, filtered and concentrated under reduced pressure to give a residue. The residue was purified by flash silica gel chromatography (40 g SepaFlash Silica Flash Column, Eluent of 0˜100% Ethyl acetate/Petroleum ether gradient @40 mL/min) to give benzyl 3-chloroisoquinoline-6-carboxylate (3.6 g, 71% yield) as a white solid. LCMS: m/z (M+H)⁺=297.9. ¹H NMR (400 MHz, CHLOROFORM-d) δ 9.17 (s, 1H), 8.55 (s, 1H), 8.23 (d, J=8.4 Hz, 1H), 8.06 (d, J=8.8 Hz, 1H), 7.85 (s, 1H), 7.59-7.35 (m, 5H), 5.47 (s, 2H).

### Step 3: benzyl 3-iodoisoquinoline-6-carboxylate

To a solution of benzyl 3-chloroisoquinoline-6-carboxylate (1.0 g, 3.4 mmol, 1 eq) and iodosodium (1.0 g, 6.7 mmol, 2 eq) in dioxane (10 mL) was added copper (1⁺) iodide (32 mg, 0.17 mmol, 0.05 eq) and methyl[2-(methylamino)ethyl]amine (30 mg, 0.34 mmol, 0.1 eq), then the mixture was stirred at 150° C. for 8 hours under N₂ in microwave reactor to give a grey suspension. The reaction mixture was filtered and the filtrate was quenched by water 20 mL and extracted with ethyl acetate 60 mL (20 mL×3).The combined organic layer was washed with brine 60 mL (20 mL×3), dried over Na₂SO₄, filtered and concentrated under reduced pressure to give a residue. The residue was purified by flash silica gel chromatography (40 g SepaFlash Silica Flash Column, Eluent of 0˜100% Ethyl acetate/Petroleum ether gradient @40 mL/min) to give benzyl 3-iodoisoquinoline-6-carboxylate (0.80 g, 22% yield) as a white solid. LCMS: m/z (M+H)⁺=389.9

### Step 4: Benzyl 3-((diethoxyphosphoryl)difluoromethyl)isoquinoline-6-carboxylate

To a solution of zinc (0.27 g, 4.1 mmol, 2 eq) in THF (8 mL) was added chlorotrimethylsilane (0.11 g, 1.0 mmol, 0.5 eq), then the mixture was stirred at 55° C. for 1.5 hours under N₂. Then a solution of diethyl (bromodifluoromethyl)phosphonate (1.1 g, 4.1 mmol, 2 eq) in THF (8 mL) was added and stirred at 55° C. for 2 hours under N₂. Then, copper(1+) bromide (0.59 g, 4.1 mmol, 2 eq) and DMA (4 mL) was added and stirred at 55° C. for 10 min. Then, a solution of benzyl 3-iodoisoquinoline-6-carboxylate (0.8 g, 2.1 mmol, 1 eq) in DMA (4 mL) was added and stirred at 55° C. for 16 hours under N₂ to give a light yellow solution. The reaction mixture was filtered and the filtrate was quenched by H₂O (20 mL) and extracted with ethyl acetate 60 mL (20 mL×3). The combined organic layers were washed with brine 60 mL (20 mL×3), dried over Na₂SO₄, filtered and concentrated under reduced pressure to give a residue. The residue was purified by prep-HPLC (column: Phenomenex Luna C18 150×25×10 um; mobile phase: [water (10 mM NH₄HCO₃)-ACN; B %: 50%-75%, 18 min). The solution was lyophilized to give benzyl 3-((diethoxyphosphoryl)difluoromethyl)isoquinoline-6-carboxylate (0.30 g, 32% yield) as a yellow solid. LCMS: m/z (M+H)⁺=450.0. ¹H NMR (400 MHz, CHLOROFORM-d) δ 9.33 (s, 1H), 8.63 (s, 1H), 8.26 (dd, J=1.2, 8.8 Hz, 1H), 8.14 (s, 1H), 8.03 (d, J=8.4 Hz, 1H), 7.46-7.40 (m, 2H), 7.39-7.25 (m, 3H), 5.38 (s, 2H), 4.38-4.10 (m, 4H), 1.31 (t, J=7.2 Hz, 6H).

### Step 5: 3-((diethoxyphosphoryl)difluoromethyl)isoquinoline-6-carboxylic Acid

To a solution of benzyl 3-((diethoxyphosphoryl)difluoromethyl)isoquinoline-6-carboxylate (0.27 g, 0.60 mmol, 1 eq) in MeOH (5 mL) was added wet Pd/C (0.15 g). The mixture was stirred at 25° C. for 3 hours under H₂ (15 psi). The reaction mixture was filtered and concentrated under reduced pressure to give crude 3-((diethoxyphosphoryl)difluoromethyl)isoquinoline-6-carboxylic acid (0.2 g, crude). LCMS: m/z (M+H)⁺=359.9

### Preparation of ((7-(((5S,8S,10aR)-3-acetyl-8-(cinnolin-6-yl(methyl)carbamoyl)-6-oxodecahydropyrrolo[1,2-a][1,5]diazocin-5-yl)carbamoyl)quinolin-2-yl)difluoromethyl)phosphonic Acid

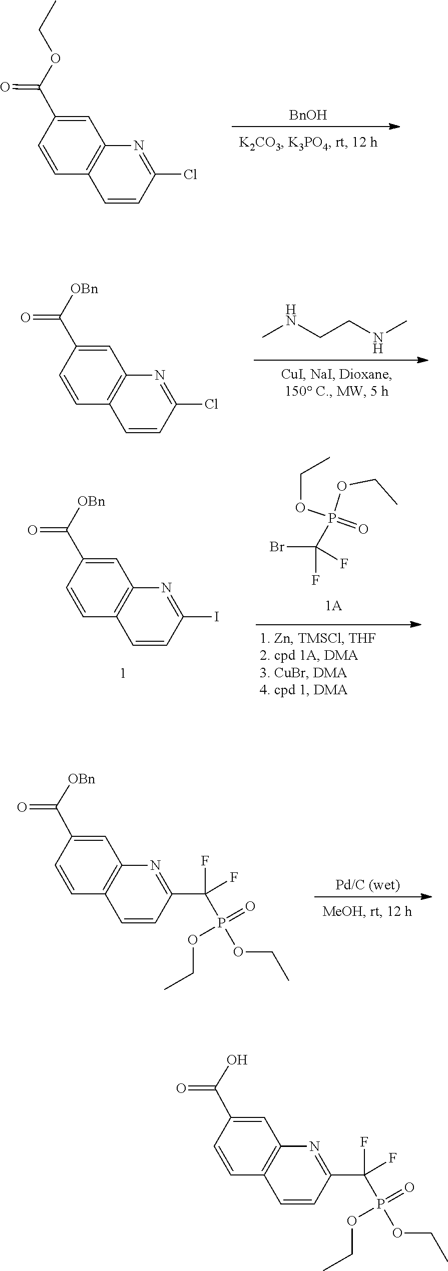

### Step 1: Benzyl 2-chloroquinoline-7-carboxylate

To a solution of ethyl 2-chloroquinoline-7-carboxylate (0.50 g, 2.1 mmol, 1 eq) in benzyl alcohol (5 mL) was added tripotassium phosphate (45 mg, 0.21 mmol, 0.1 eq) and potassium carbonate (0.30 g, 2.1 mmol, 1 eq), then the mixture was stirred at 25° C. for 12 hours. The residue was diluted with water (20 mL) and extracted with ethyl acetate (40 mL×2), the combined organic layers were washed with saturated brine (20 mL×2), dried over Na₂SO₄, filtered and concentrated under reduced pressure to give a residue. The residue was purified by column chromatography (SiO₂, Petroleum ether/Ethyl acetate=100:0 to 90:10) to give benzyl 2-chloroquinoline-7-carboxylate (0.39 g, 62% yield) as a white solid. ¹H NMR (400 MHz, CHLOROFORM-d) δ 8.80 (s, 1H), 8.24-8.12 (m, 2H), 7.89 (d, J=8.4 Hz, 1H), 7.52-7.47 (m, 3H), 7.46-7.35 (m, 3H)

### Step 2: Benzyl 2-iodoquinoline-7-carboxylate

To a solution of benzyl 2-chloroquinoline-7-carboxylate (0.3 g, 0.87 mmol, 1 eq) and sodium iodide (0.26 g, 1.7 mmol, 2 eq) in dioxane (38 mL) was added copper(1⁺) iodide (8.3 mg, 44 μmol, 0.05 eq), then the mixture was stirred at 150° C. for 5 hours under microwave as a grey suspension. The reaction mixture was filtered and the filtrate was concentrated to give a residue. The residue was purified by column chromatography (SiO₂, Petroleum ether/Ethyl acetate=10:1 to 3:1) to give benzyl 2-iodoquinoline-7-carboxylate (0.1 g, 29% yield) as a white solid. LCMS: m/z (M+H)⁺=389.9

### Step 3: Benzyl 2-((diethoxyphosphoryl)difluoromethyl)quinoline-7-carboxylate

To a suspension of zinc (47 mg, 0.72 mmol, 2 eq) in tetrahydrofuran (2 mL) was added chlorotrimethylsilane (16 mg, 0.14 mmol, 0.4 eq) under N₂, then the reaction mixture was stirred at 55° C. for 1.5 hours, then a solution of diethyl (bromodifluoromethyl)phosphonate (0.19 g, 0.72 mmol, 2 eq) in tetrahydrofuran (2 mL) was added dropwise (temperature kept between 50-55° C.). The reaction mixture was stirred at 55° C. for 2 hours (the clouded suspension became a solution) and copper (1+) bromide (51 mg, 0.36 mmol, 1 eq) in dimethylacetamide (2 mL) were added and the mixture was stirred at 55° C. for 10 min. A solution of benzyl 2-iodoquinoline-7-carboxylate (0.1 g, 0.36 mmol, 1 eq) in dimethylacetamide (2 mL) was added and the reaction mixture was stirred at 55° C. for 16 hours to give a green suspension. The reaction mixture was concentrated under reduced pressure to remove the THF. The mixture was purified by prep-HPLC (column: Phenomenex Luna C18 150×25 mm×10 um, mobile phase: water (0.225% FA)-ACN; B %: 54%-84%, 10 min) and lyophilized to give benzyl 2-((diethoxyphosphoryl)difluoromethyl)quinoline-7-carboxylate (40 mg, 30% yield) as a yellow oil. ¹H NMR (400 MHz, CHLOROFORM-d) δ 8.95 (s, 1H), 8.36 (d, J=8.4 Hz, 1H), 8.31-8.22 (m, 1H), 7.95 (d, J=8.8 Hz, 1H), 7.53-7.49 (m, 2H), 7.46-7.35 (m, 4H), 5.46 (s, 2H), 4.40-4.31 (m, 4H), 1.39 (t, J=7.2 Hz, 6H)

### Step 4: 2-((diethoxyphosphoryl)difluoromethyl)quinoline-7-carboxylic Acid

To a solution of benzyl 2-((diethoxyphosphoryl)difluoromethyl)quinoline-7-carboxylate (40 mg, 89 μmol, 1 eq) in methanol (1 mL) and tetrahydrofuran (1 mL) was added wet Pd/C (40 mg) under N₂, the mixture was stirred at 25° C. for 2 hours under H₂ (15 psi). The reaction mixture was filtered and the filtrate was concentrated to give 2-((diethoxyphosphoryl)difluoromethyl)quinoline-7-carboxylic acid (15 mg, crude) as a yellow oil. LCMS: m/z (M+H)⁺=360.1

### Synthesis of Building Blocks

### Synthesis of 6-phenyl-4-azaspiro[2.4]heptane

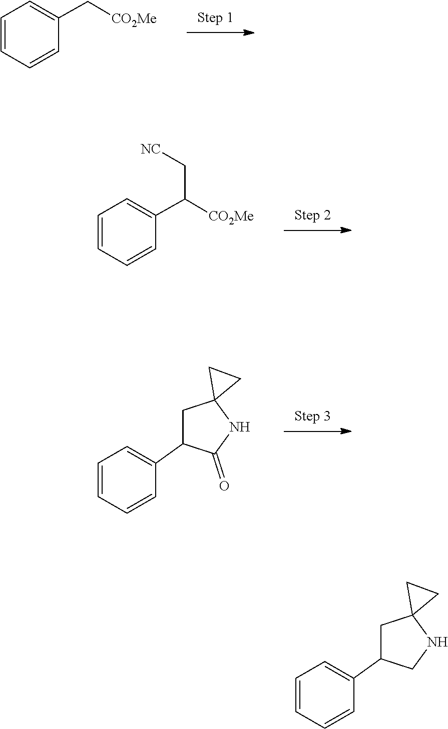 [SMILES: COC(=O)Cc1ccccc1 ; COC(=O)C(CC#N)c1ccccc1 ; O=C1NC2(CC2)CC1c1ccccc1 ; c1ccc(C2CNC3(CC3)C2)cc1]

### Step 1: Preparation of Methyl 3-cyano-2-phenylpropanoate

To a cooled (−78° C.) solution of methyl 2-phenylacetate (5.0 g, 33.3 mmol, 1.0 eq) in dry THF (50 mL) was slowly added a solution of 2 M LDA (20 mL, 40.0 mmol, 1.2 eq) in THF. After 1 h, 2-bromoacetonitrile (4.2 g, 35.0 mmol, 1.1 eq) slowly in a dropwise manner and the reaction was further aged for additional 1 h at −78° C. To the mixture was added saturated aqueous NH₄Cl (5 mL) and the mixture was warmed to room temperature. The mixture was with EtOAc (20 mL×3). The organic layers were combined, washed with brine (150 mL), dried over anhydrous Na₂SO₄, and concentrated under reduced pressure. The residue was purified by flash column chromatography on silica gel to methyl 3-cyano-2-phenylpropanoate (5.0 g, 26.5 mmol, 79%) as a white solid. LCMS (ESI) m/z=190.1 [M+H]⁺.

### Step 2: Preparation of 6-phenyl-4-azaspiro[2.4]heptan-5-one

To a cooled (0° C.) solution of methyl 3-cyano-2-phenylpropanoate (5.0 g, 26.5 mmol, 1.0 eq) and Ti(OiPr)₄ (9.0 g, 31.7 mmol, 1.2 eq) in dry THF (100 mL) was slowly added a 3 M solution of EtMgBr (20 mL, 59.6 mmol, 2.25 eq), while maintaining inner temperature between −5° C. to 0° C. After complete addition of EtMgBr, the mixture was stirred at 0° C. for additional 1 h. The reaction mixture was quenched by addition of aqueous 2 N HCl (60 mL). The resulting acidic solution was extracted with EtOAc (50 mL×3). The combined organic layers were washed with brine (20 mL×2), dried over anhydrous Na₂SO₄, and concentrated under reduced pressure. The residue was purified by flash column chromatography on to afford 6-phenyl-4-azaspiro[2.4]heptan-5-one (2.1 g, 11.2 mmol, 42% yield) as a white solid. LCMS (ESI) m/z=188 [M+H]⁺; ¹H NMR (400 MHz, CDCl₃) δ 7.39-7.24 (m, 6H), 3.86 (dd, J=9.4, 7.6 Hz, 1H), 2.51 (dd, J=12.9, 9.4 Hz, 1H), 2.29 (dd, J=12.9, 7.6 Hz, 1H), 0.85-0.88 (m, 1H), 0.87-0.81 (m, 1H), 0.76-0.64 (m, 2H).

### Step 3: Preparation of 6-phenyl-4-azaspiro[2.4]heptane

To a cooled (10° C.) solution of 6-phenyl-4-azaspiro[2.4]heptan-5-one (2.1 g, 11.2 mmol, 1.0 eq) in dry THF (50 mL) was added NaBH₄ (2.1 g, 56.0 mmol, 5.0 eq) in several portions. To the mixture was added in a dropwise manner BF₃·Et₂O (6.7 mL, 56.0 mmol, 5.0 eq). The reaction mixture was heated at 60° C. for 16 h. The mixture was cooled to ambient temperature and aqueous 2 N HCl (30 mL) was introduced slowly. The acidic mixture was extracted with EtOAc (30 mL×3). The organic layers were combined, washed with brine (20 mL), dried over anhydrous Na₂SO₄, and concentrated under reduced pressure. The residue was purified by Biotage® C18 column to afford 6-phenyl-4-azaspiro[2.4]heptane (1.5 g, 8.7 mmol, 78%) as a colorless oil. LCMS (ESI) m/z=174 [M+H]⁺; ¹H NMR (400 MHz, CDCl₃) 8.59 (s, 1H), δ 7.38-7.27 (m, 5H), 3.85-3.57 (m, 2H), 3.40-3.17 (m, 1H), 2.43-2.09 (m, 2H), 1.46-1.18 (m, 2H), 0.98-0.66 (m, 2H).

### Pyrrolidine Building Block Synthesis:

### Synthesis of rel-(trans)-4-cyclohexylpyrrolidine-3-carbonitrile

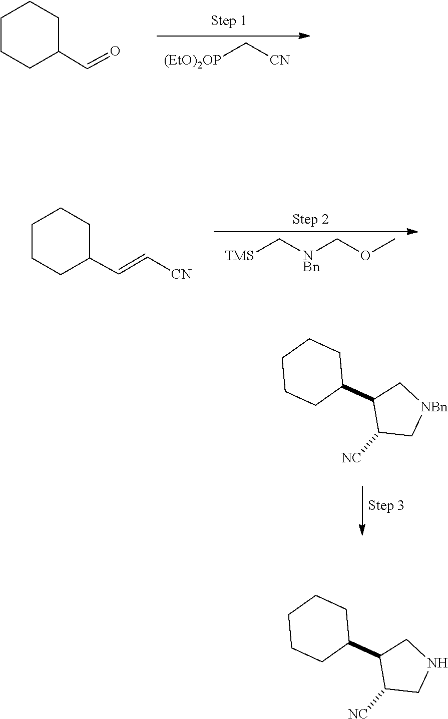

### Step 1: Preparation of (E)-3-cyclohexylacrylonitrile

To a solution of cyclohexanecarbaldehyde (500 mg, 4.45 mmol, 1.0 eq) in THF (15 mL) were added t-BuOK (998 mg, 8.90 mmol, 2.0 eq) and diethyl (cyanomethyl)phosphonate (788 mg, 4.45 mmol, 1.0 eq) at room temperature. The solution was stirred at room temperature for 1 h. After completion, the reaction mixture was quenched by adding H₂O (10 mL), then extracted with EtOAc (10 mL×3). The organic layers were combined and washed with brine (10 mL), dried over anhydrous Na₂SO₄, filtered and concentrated under reduced pressure. The residue was purified by flash column chromatography on silica gel to afford (E)-3-cyclohexylacrylonitrile (300 mg, 2.21 mmol, 50% yield) as a colorless oil. LC-MS (ESI) m/z=136 [M+H]⁺.

### Step 2: Preparation of rel-(trans)-1-benzyl-4-cyclohexylpyrrolidine-3-carbonitrile

To a solution of (E)-3-cyclohexylacrylonitrile (300 mg, 2.21 mmol, 1.0 eq) in CH₂Cl₂ (5 mL) were added N-benzyl-1-methoxy-N-((trimethylsilyl)methyl)methanamine (524 mg, 2.21 mmol, 1.0 eq) and TFA (25.1 mg, 221 μmol, 0.1 eq). The reaction mixture was stirred at room temperature for 12 h. After completion, the reaction mixture was dilute with CH₂Cl₂ (10 mL) and washed with aqueous saturated NaHCO₃ (5 mL), the organic layer was separated and concentrated under reduced pressure to afford crude rel-(trans)-1-benzyl-4-cyclohexylpyrrolidine-3-carbonitrile (700 mg) as a colorless oil. LC-MS (ESI) m/z=269 [M+H]⁺.

### Step 3: Preparation of rel-(trans)-4-cyclohexylpyrrolidine-3-carbonitrile

To a solution of (trans)-1-benzyl-4-cyclohexylpyrrolidine-3-carbonitrile (700 mg) in dry 1,2-dichloroethane (15 mL) was added 1-chloroethyl carbonochloridate (3.71 g, 26.0 mmol, 10.0 eq). The resulting mixture was stirred at 70° C. for 12 h, then concentrated under reduced pressure, the crude product was dissolved in MeOH (5 mL) and stirred at 70° C. for 1 hr. After completion, the reaction solution was cooled to room temperature and concentrated under reduced pressure. The residue was purified by flash column chromatography on silica gel to afford rel-(trans)-4-cyclohexylpyrrolidine-3-carbonitrile (211 mg, 1.18 mmol, 46% yield) as a colorless oil. LC-MS (ESI) m/z=179 [M+H]⁺.

The following intermediates in Table 10 were prepared according to the representative procedures (Step 1 through Step 3) described for rel-(trans)-4-cyclohexylpyrrolidine-3-carbonitrile utilizing appropriate starting materials and modifications. Compounds were prepared as racemates with trans stereochemical configuration relative to the C3 and C4 stereocenters within the pyrrolidine ring.

TABLE 10

| Name | Structure | LCMS |
|---|---|---|
| rel-(trans)-4-(2-oxo- 1,2-dihydropyridin- 4-yl)pyrrolidine-3- carbonitrile | 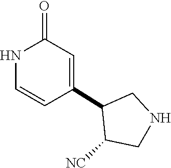 [SMILES: N#C[C@H]1CNC[C@@H]1c1cc[nH]c(=O)c1] | 190 [M + H]⁺ |
| rel-(trans)-3-methyl-4- phenylpyrrolidine-3- carbonitrile | 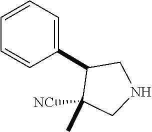 | 187.2 [M + H]⁺ |

tert-Butyl rel-(trans)-3-cyano-4-phenylpyrrolidine-1-carboxylate and tert-butyl (trans)-3-cyano-4-(2-oxo-1,2-dihydropyridin-4-yl)pyrrolidine-1-carboxylate were prepared according to the method describe above for the synthesis of rel-(trans)-4-cyclohexylpyrrolidine-3-carbonitrile. The racemic mixture of trans-isomers were purified under SFC conditions and the absolute stereochemistry was arbitrarily assigned as drawn.

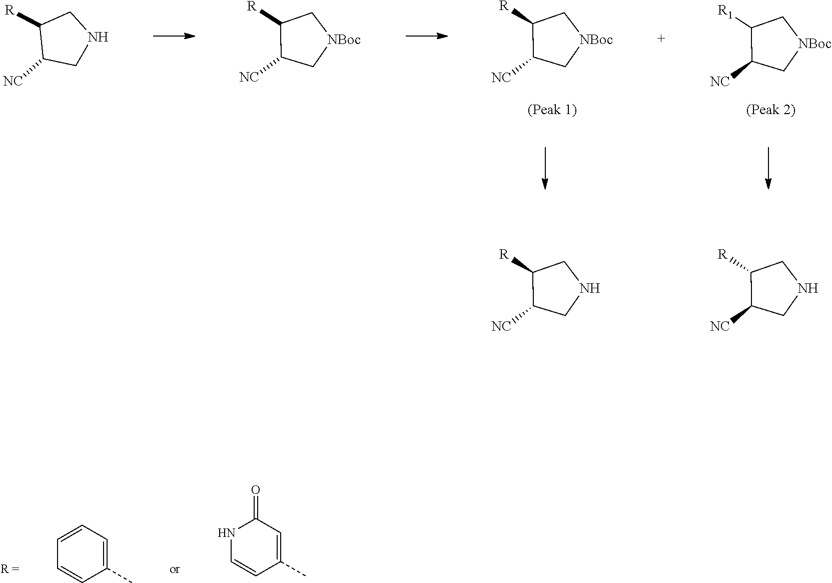

### Preparative Separation Method:

Instrument: Waters Thar 80 preparative SFC; Column: ChiralPak C-IG, 100×4.6 mm I.D., 5 μm; Mobile phase: A for CO₂ and B for methanol (0.05% diethylamine); Gradient: 10% to 40% B in 8 min; Flow rate: 2.5 mL/min; Back pressure: 100 bar, Column temperature: 40° C.; Wavelength: 210 nm; Cycle-time: 2 min

The following intermediates in Table 11 were prepared according to the procedure described for rel-(trans)-4-cyclohexylpyrrolidine-3-carbonitrile utilizing appropriate starting materials and modifications.

TABLE 11

| Name | Structure | LCMS |
|---|---|---|
| tert-butyl rel-(trans)-3- cyano- 4-phenylpyrrolidine-1- carboxylate (Peak 1) | 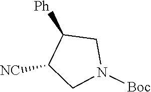 [SMILES: CC(C)(C)OC(=O)N1C[C@H](c2ccccc2)[C@@H](C#N)C1] | 217 [(M − 56) + 1]⁺ |
| tert-butyl rel-(trans)-3- cyano-4- phenylpyrrolidine-1- carboxylate (Peak 2) | 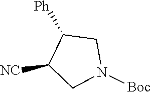 [SMILES: CC(C)(C)OC(=O)N1C[C@@H](C#N)[C@H](c2ccccc2)C1] | 217 [(M − 56) + 1]⁺ |
| tert-butyl rel-(trans)-3- cyano-4-(2-oxo-1,2- dihydropyridin-4-yl) pyrrolidine-1-carboxylate (Peak 1) | 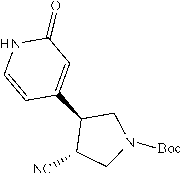 [SMILES: CC(C)(C)OC(=O)N1C[C@H](c2cc[nH]c(=O)c2)[C@@H](C#N)C1] | 190 [M + H]⁺ |
| tert-butyl rel-(trans)-3- cyano-4-(2-oxo-1,2- dihydropyridin-4-yl) pyrrolidine-1-carboxylate (Peak 2) | 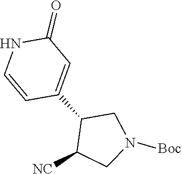 [SMILES: CC(C)(C)OC(=O)N1C[C@@H](C#N)[C@H](c2cc[nH]c(=O)c2)C1] | 190 [M + H]⁺ |

### Synthesis of 2-methyl-2-(4-(methylamino)phenyl)propanenitrile

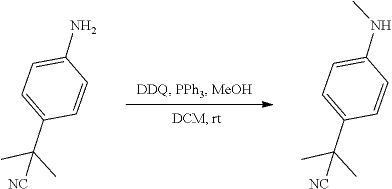

A solution of 2-(4-aminophenyl)-2-methylpropanenitrile (360 mg, 2.24 mmol, 1 eq.), PPh₃ (587 mg, 2.24 mmol, 1 eq)., 4,5-dichloro-3,6-dioxocyclohexa-1,4-diene-1,2-dicarbonitrile (508 mg, 2.24 mmol, 1 eq.) and MeOH (71.6 mg, 2.24 mmol, 1.0 eq.) in DCM (10 mL) was stirred at room temperature for 1 hr under nitrogen. After completion, the reaction was filtered and concentrated, providing a residue, which was purified by flash column chromatography on silica gel to afford 2-methyl-2-(4-(methylamino)phenyl)propanenitrile (90.0 mg, 516 μmol, 23%) as a white solid. LCMS (ESI): m/z=175.3 [M+H]⁺.

The following intermediates in Table 12 were prepared according to the procedure described for 2-methyl-2-(4-(methylamino)phenyl)propanenitrile utilizing appropriate starting materials and modifications.

TABLE 12

| Name | Structure | LCMS |
|---|---|---|
| N-methyl-4- (2-phenylpropan-2-yl) aniline | 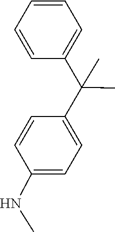 [SMILES: CNc1ccc(C(C)(C)c2ccccc2)cc1] | 226.3 [M + H]⁺ |
| N-methyl-4- (tert-pentyl) aniline | 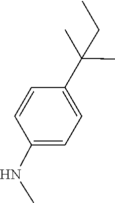 | 178 [M + H]⁺ |
| 4-(tert-butyl)-3- chloro-N- methylaniline | 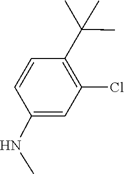 | 198 [M + H]⁺ |

### Synthesis of 2-methyl-2-(4-methylamino)phenyl)propan-1-ol

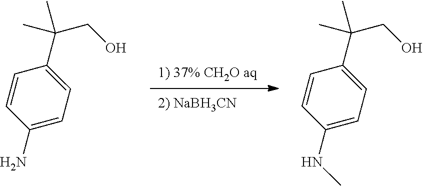

A solution of 2-(4-aminophenyl)-2-methylpropan-1-ol (230 mg, 1.39 mmol, 1 eq.), formaldehyde (112 mg, 1.39 mmol, 1 eq.), sodium cyanoborohydride (87.3 mg, 1.39 mmol, 1 eq.) in MeOH (20 mL) was stirred at room temperature overnight under nitrogen. After completion, the reaction was filtered and concentrate. The residue was purified by C18 column to afford 2-methyl-2-[4-(methylamino)phenyl]propan-1-ol (60.0 mg, 334 μmol, 24%) as a white solid. LCMS (ESI): m/z=180.2 [M+H]⁺.

The following intermediates in Table 13 were prepared according to the procedure described for 2-methyl-2-(4-(methylamino)phenyl)propan-1-ol utilizing appropriate starting materials and modifications.

TABLE 13

| Name | Structure | LCMS |
|---|---|---|
| N-methylbenzo[d] isoxazol-5-amine | 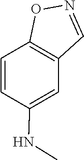 | 149.2 [M + H]⁺ |

### Synthesis of Intermediate: (rel-trans)-5-phenylpiperidine-3-carbonitrile

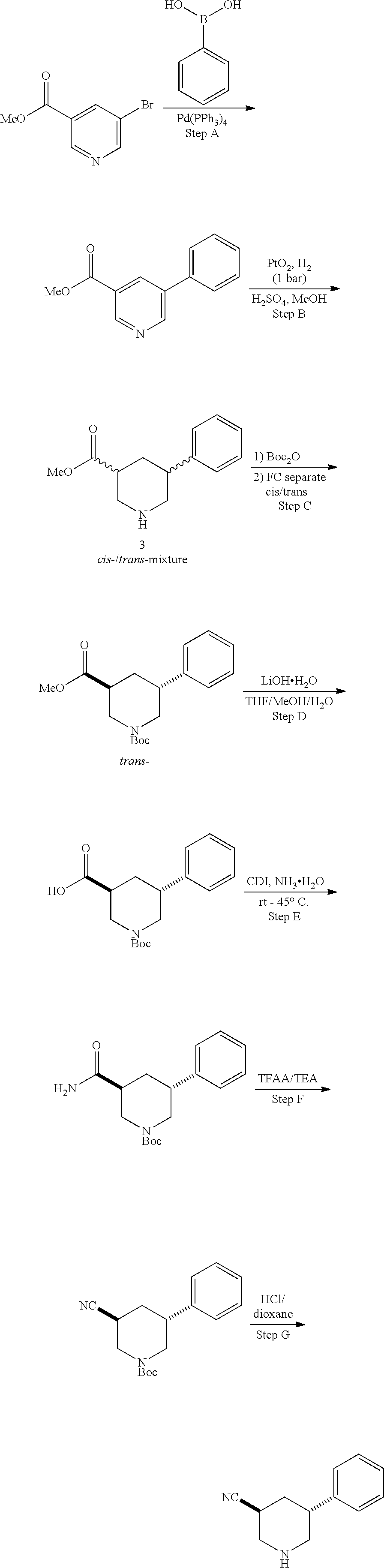

### Step A: Methyl 5-Phenylnicotinate

An aqueous solution of Na₂CO₃ (1.96 g, 18.5 mmol, 2.0 eq.) in H₂O (10 mL) and a solution of phenylboronic acid (1.34 g, 11.0 mmol, 1.2 eq.) in MeOH (5 mL) were added to a stirred solution of methyl 5-bromonicotinate (2.0 g, 9.25 mmol, 1 eq.) and Pd(PPh₃)₄(320 mg, 277 μmol, 0.03 eq.) in toluene (20 mL) under nitrogen. The stirred mixture was stirred at 80° C. for 4 hrs. The reaction mixture was cooled to room temperature and extracted with EtOAc (60 mL). The solvent was removed under reduced pressure. The residue was purified by flash chromatography on silica gel to afford methyl 5-phenylnicotinate (1.30 g, 6.09 mmol, 61%) as a white solid. ¹H NMR (400 MHz, CDCl₃) δ 9.19 (d, J=1.9 Hz, 1H), 9.01 (d, J=2.3 Hz, 1H), 8.49 (t, J=2.1 Hz, 1H), 7.68-7.57 (m, 2H), 7.56-7.35 (m, 3H), 3.99 (s, 3H).

### Step B: Methyl 5-phenylpiperidine-3-carboxylate

To a solution of methyl 5-phenylnicotinate (1.0 g, 4.68 mmol, 1 eq.) and H₂SO₄ (0.05 mL) in MeOH (10 mL) was added PtO₂ (100 mg, 10%). The suspension was degassed under vacuum and purged with H₂ three times. The mixture was stirred under H₂ (balloon) at room temperature for 16 hrs. The reaction mixture was filtered by celite. The filtrate was concentrated under reduced pressure to afford methyl 5-phenylpiperidine-3-carboxylate (1 g, 4.56 mmol, 98%) as a white solid. LCMS (ESI) m/z=220 [M+H]⁺.

### Step C: 1-(tert-butyl) 3-methyl (rel-trans)-5-phenylpiperidine-1,3-dicarboxylate

To a solution of methyl 5-phenylpiperidine-3-carboxylate (1.0 g, 4.56 mmol, 1 eq.) in THF (10 mL) were added TEA (1.37 g, 13.6 mmol, 3.0 eq.) and di-tert-butyl dicarbonate (1.49 g, 6.84 mmol, 1.5 eq.) at room temperature. After addition, the reaction mixture was stirred at room temperature overnight. The reaction mixture was concentrated under reduced pressure and the resulting residue was purified by flash chromatography on silica gel (cis and trans isomers were separable) to afford 1-(tert-butyl) 3-methyl (rel-trans)-5-phenylpiperidine-1,3-dicarboxylate (800 mg, 2.50 mmol, 55%) as a colorless oil. LCMS (ESI) m/z=320 [M+H]⁺. ¹H NMR (400 MHz, CDCl₃) δ 7.34-7.23 (m, 5H), 4.46-3.96 (m, 2H), 3.72 (s, 31H), 3.29-2.89 (m, 3H), 2.81-2.64 (m, 1H), 2.44-2.24 (m, 1H), 1.98-1.83 (m, 1H), 1.45 (s, 9H).

### Step D: (rel-trans)-1-(tert-butoxycarbonyl)-5-phenylpiperidine-3-carboxylic Acid

To a solution of 1-(tert-butyl) 3-methyl (rel-trans)-5-phenylpiperidine-1,3-dicarboxylate (120 mg, 375 μmol, 1 eq.) in a mixture of THF (4 mL), H₂O (1 mL) and MeOH (1 mL) was added LiOH—H₂O (78.4 mg, 1.87 mmol, 5.0 eq.) at room temperature. After addition, the reaction mixture was stirred at room temperature overnight. The reaction mixture was cooled to 0° C. and acidified with 2.0 M aqueous HCl solution to pH=3. The mixture was extracted with EtOAc (30 mL). The organic phase was dried over Na₂SO₄, filtered and concentrated under reduced pressure to afford (rel-trans)-1-(tert-butoxycarbonyl)-5-phenylpiperidine-3-carboxylic acid (110 mg, 360 μmol, 96.4%) as a colorless oil. LCMS (ESI): m/z=304 [M−H]⁻.

### Step E: tert-butyl (rel-trans)-3-carbamoyl-5-phenylpiperidine-1-carboxylate

A solution of (rel-trans)-1-(tert-butoxycarbonyl)-5-phenylpiperidine-3-carboxylic acid (110 mg, 360 μmol, 1 eq.) and CDI (64 mg, 396 μmol, 1.1 eq.) in EtOAc (4 mL) was stirred at room temperature for 2 hrs. Then, NH₃·H₂O (1 mL) was added. The reaction mixture was stirred at 45° C. for 2 hrs. The reaction mixture was cooled to room temperature and extracted with EtOAc (15 mL). The mixture was washed with water (5 mL). The organic phase was concentrated under reduced pressure to give a residue. The residue was purified by C18 column to afford tert-butyl (rel-trans)-3-carbamoyl-5-phenylpiperidine-1-carboxylate (80 mg, 262 μmol, 73%) as a white solid. LCMS (ESI): m/z=305.2[M+H]⁺.

### Step F: tert-butyl (rel-trans)-3-cyano-5-phenylpiperidine-1-carboxylate

To a solution of tert-butyl (rel-trans)-3-carbamoyl-5-phenylpiperidine-1-carboxylate (80 mg, 262 μmol, 1 eq.) in DCM (2 mL) was added TEA (66 mg, 655 μmol, 2.5 eq.) at room temperature. The mixture was cooled to 0° C. and TFAA (71 mg, 340 μmol, 1.3 eq.) was added dropwise. After addition, the reaction mixture was stirred at room temperature for 16 hrs. The reaction mixture was diluted with DCM (15 mL). The mixture was washed with water (5 mL). The organic phase was concentrated under reduced pressure to give a residue. The residue was purified by C18 column to give tert-butyl (rel-trans)-3-cyano-5-phenylpiperidine-1-carboxylate (57 mg, 199 μmol, 76%) as a white solid. LCMS (ESI): m/z=309 [M+Na]⁺.

### Step G: (rel-trans)-5-phenylpiperidine-3-carbonitrile

A solution of tert-butyl (rel-trans)-3-cyano-5-phenylpiperidine-1-carboxylate (57 mg, 199 μmol, 1 eq.) in 4.0 M HCl/dioxane (2 mL) was stirred at room temperature for 1 hr. The reaction mixture was concentrated under reduced pressure to give (rel-trans)-5-phenylpiperidine-3-carbonitrile (35 mg, 187 μmol, 95%) as a white solid which was used in next step without purification. LCMS (ESI): m/z=187 [M+H]⁺.

### Preparation of (3S,5R)-5-benzylpyrrolidine-3-carbonitrile

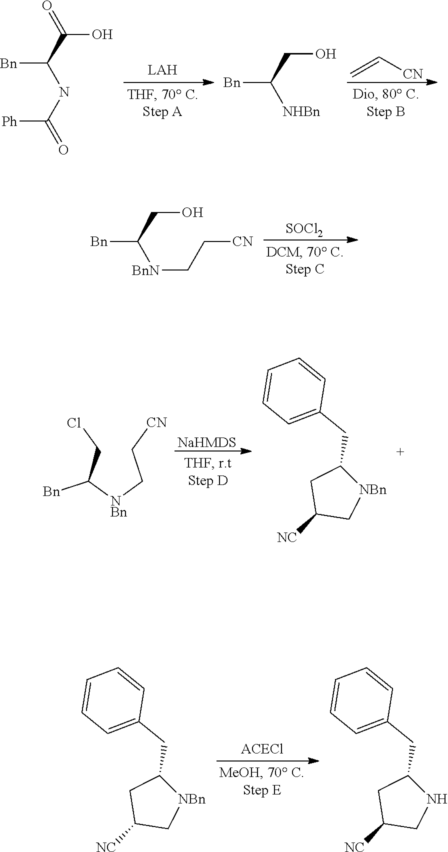

### Step A: (S)-2-(benzylamino)-3-phenylpropan-1-ol

Benzoyl-L-phenylalanine (1 g, 3.71 mmol, 1 eq.) was added drop-wisely to LiAlH₄ (421 mg, 11.1 mmol, 3 eq.) in THF (30 mL) at room temperature and the resulting mixture was stirred at 70° C. for 18 hrs. After completion, the reaction was quenched by Na₂SO₄-10H₂O, filtered and the filter cake was washed by DCM (50 mL). The filtrate was concentrated in vacuo. The residue was purified by flash column chromatography on silica gel to afford (S)-2-(benzylamino)-3-phenylpropan-1-ol (700 mg, 2.90 mmol, 78%) as a white solid. LCMS (ESI): m/z=242.2 [M+H]⁺.

### Step B: (S)-3-(benzyl(1-hydroxy-3-phenylpropan-2-yl)amino)propanenitrile

To (S)-2-(benzylamino)-3-phenylpropan-1-ol (650 mg, 2.69 mmol, 1 eq.) and prop-2-enenitrile (9.97 g, 188 mmol, 70 eq.) in 1,4-dioxane (15 mL) was added AcOH (322 mg, 5.38 mmol, 2 eq.) at room temperature, then the solution was stirred at 80° C. for 48 hrs. After completion, the solvent was removed under reduced pressure and the residue was purified by flash column chromatography on silica gel to provide (S)-3-(benzyl(1-hydroxy-3-phenylpropan-2-yl)amino)propanenitrile (560 mg, 1.90 mmol, 71%) as a white solid. LCMS (ESI): m/z=295.2 [M+H]⁺.

### Step C: (S)-3-(benzyl(1-chloro-3-phenylpropan-2-yl)amino)propanenitrile

SOCl₂ (553 mg, 4.65 mmol, 2.5 eq.) was added to (S)-3-(benzyl(1-hydroxy-3-phenylpropan-2-yl)amino)propanenitrile (550 mg, 1.86 mmol, 1 eq.) in DCM (10 mL) at room temperature, then the solution was stirred at 70° C. for 0.5 hr. After completion, the reaction was cooled to room temperature, quenched with NaHCO₃(aq.) (2 mL), extracted with EtOAc (10 mL×2), The combined organic layers were washed with brine and dried over Na₂SO₄, filtered and concentrated under reduced pressure to give crude (S)-3-(benzyl(1-chloro-3-phenylpropan-2-yl)amino)propanenitrile (550 mg, 1.75 mmol, 95%) as oil. LCMS(ESI): m/z=313.2 [M+H]⁺.

### Step D: (3R,5S)-1,5-dibenzylpyrrolidine-3-carbonitrile

To (S)-3-(benzyl(1-chloro-3-phenylpropan-2-yl)amino)propanenitrile (550 mg, 1.75 mmol, 1 eq.) in THF (20 mL) was added NaHMDS (3 mL, 1.2 eq.) at room temperature under nitrogen, then the solution was stirred at room temperature for 0.5 hr. After completion, the reaction was quenched with NH₄Cl(aq.) (0.5 mL), extracted with EtOAc (5 mL×2). The combined organic layers were washed with brine, dried over Na₂SO₄, filtered and concentrated under reduced pressure. The residue was purified by flash column chromatography on silica gel to give (3S,5R)-1,5-dibenzylpyrrolidine-3-carbonitrile (150 mg, 0.54 mmol, 31%) as oil and (3R,5R)-1,5-dibenzylpyrrolidine-3-carbonitrile (250 mg, 0.90 mmol, 52%). LCMS(ESI): m/z=277.2 [M+H]⁺.

### Step E: (3R,5S)-5-benzylpyrrolidine-3-carbonitrile

To a solution of (3S,5R)-1,5-dibenzylpyrrolidine-3-carbonitrile (150 mg, 0.5427 mmol, 1 eq.) in DCE (10 mL) was added 1-chloroethyl chloroformate (387 mg, 2.71 mmol, 5 eq.) at room temperature, then stirred at 80° C. for 18 hrs. The solution was concentrated, then the residue was dissolved in MeOH (10 mL) and the solution was stirred at 70° C. for 2 hrs. After completion, the solution was concentrated, then the residue was purified by flash column chromatography on silica gel to afford (3S,5R)-5-benzylpyrrolidine-3-carbonitrile (50.0 mg, 0.27 mmol, 50%). LCMS (ESI): m/z=187.2 [M+H]⁺. ¹H NMR (400 MHz, CDCl₃) δ 7.34-7.16 (m, 5H), 3.35-3.24 (m, 2H), 3.19-3.10 (m, 1H), 2.96-2.72 (m, 3H), 2.32-2.20 (m, 1H), 1.79-1.63 (m, 1H).

### Step E (cis): (3R,5R)-5-benzylpyrrolidine-3-carbonitrile

1-Chloroethyl chloroformate (516 mg, 3.61 mmol, 5 eq,) was added to a solution of (3R,5R)-1,5-dibenzylpyrrolidine-3-carbonitrile (200 mg, 0.72 mmol, 1 eq.) in DCM (10 mL) at room temperature., then the solution was stirred at 80° C. for 18 hrs. After completion, the solution was concentrated, then the residue was dissolved in MeOH (10 mL) and the solution was stirred at 70° C. for 2 hrs. The solution was concentrated under reduced pressure and the residue was purified by flash column chromatography on silica gel to give (3R,5R)-5-benzylpyrrolidine-3-carbonitrile (30.0 mg, 0.16 mmol, 22%) LCMS (ESI): m/z=187.2 [M+H]⁺. ¹H NMR (400 MHz, CDCl₃) δ 7.34-7.16 (m, 5H), 3.56-3.43 (m, 1H), 3.42-3.30 (m, 1H), 3.11-3.01 (m, 1H), 2.99-2.88 (m, 1H), 2.76 (d, J=6.7 Hz, 2H), 2.22-2.10 (m, 1H), 1.85-1.73 (m, 1H).

### Preparation of (rel-trans)-4-phenylpiperidine-3-carbonitrile

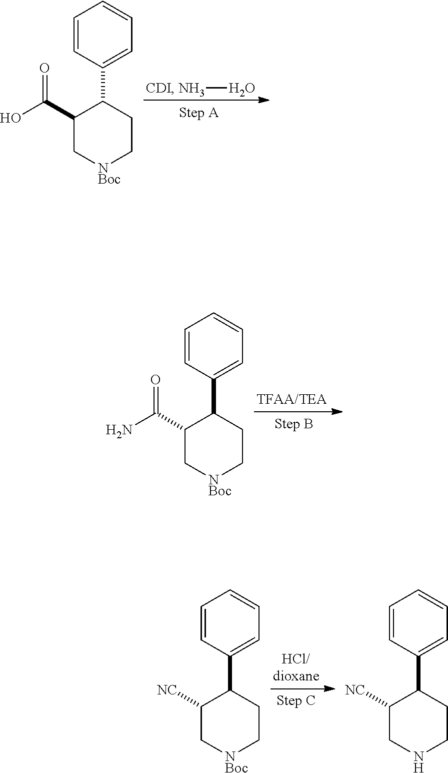

### Step A: tert-butyl (rel-trans)-3-carbamoyl-4-phenylpiperidine-1-carboxylate

A solution of (rel-trans)-1-(tert-butoxycarbonyl)-4-phenylpiperidine-3-carboxylic acid (400 mg, 1.30 mmol, 1 eq.) and CDI (231 mg, 1.43 mmol, 1.1 eq.) in EtOAc (5 mL) was stirred at room temperature for 2 hrs. Then, NH₃—H₂O (2 mL) was added. The reaction mixture was stirred at 45° C. for 2 hrs. After completion, the reaction mixture was cooled to room temperature, then diluted with H₂O (5 mL), extracted with EtOAc (10 mL×3). The organic layers were combined and washed with brine (10 mL), dried over anhydrous Na₂SO₄, and concentrated under reduced pressure. The residue was purified by C18 column to give tert-butyl (rel-trans)-3-carbamoyl-4-phenylpiperidine-1-carboxylate (390 mg, 1.28 mmol, 99%) as a white solid. LCMS (ESI) m/z=305 [M+H]⁺.

### Step B: tert-butyl (rel-trans)-3-cyano-4-phenylpiperidine-1-carboxylate

To a solution of tert-butyl (rel-trans)-3-carbamoyl-4-phenylpiperidine-1-carboxylate (390 mg, 1.28 mmol, 1 eq.) in DCM (5 mL) was added TEA (323 mg, 3.20 mmol, 2.5 eq.) at room temperature. The mixture was cooled to 0° C. and TFAA (348 mg, 1.66 mmol, 1.3 eq.) was added drop-wisely. After addition, the reaction mixture was stirred at room temperature for 16 hrs. After completion, the reaction mixture was diluted with H₂O (5 mL), extracted with DCM (5 mL×3). The organic layers were combined and washed with brine (10 mL), dried over anhydrous Na₂SO₄, and concentrated under reduced pressure. The residue was purified by C18 column to afford tert-butyl (rel-trans)-3-cyano-4-phenylpiperidine-1-carboxylate (160 mg, 558 μmol, 44%) as a white solid. LCMS (ESI) m/z=309 [M+Na].

### Step C: (rel-trans)-4-phenylpiperidine-3-carbonitrile Hydrochloride

A solution of (rel-trans)-3-cyano-4-phenylpiperidine-1-carboxylate (80 mg, 279 μmol, 1 eq.) in 4.0 M HCl/dioxane (2 mL) was stirred at room temperature for 1 hr. After completion, the reaction mixture was concentrated under reduced pressure to give (rel-trans)-4-phenylpiperidine-3-carbonitrile hydrochloride (52 mg, 279 μmol, 100%) as a white solid, which was used in next step directly without purification. LCMS: m/z=187[M+H]⁺.

### Preparation of (rel-trans)-3-(difluoromethyl)-4-phenylpyrrolidine

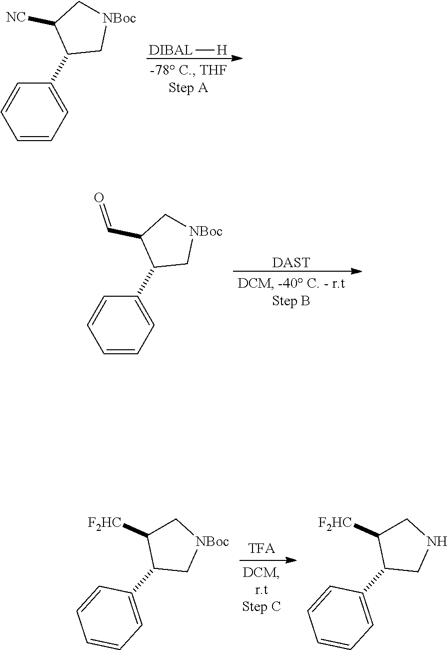

### Step A: tert-butyl (rel-trans)-3-formyl-4-phenylpyrrolidine-1-carboxylate

A solution of DIBAL-H (3.66 mL, 3.66 mmol, 2 eq.) was added into a solution of tert-butyl (rel-trans)-3-cyano-4-phenylpyrrolidine-1-carboxylate (500 mg, 1.83 mmol, 1 eq.) in THF (10 mL) via a syringe over a period of 10 min at −78° C. under N₂. Then the mixture was stirred at −78° C. for 1 hr. After completion, NH₄Cl(1 mL, aq.) solution was added drop-wisely into the reaction mixture once the reaction mixture was allowed to −60 to −20° C. The residue was partitioned between EtOAc (20 mL) and 1 N aq. HCl (10 mL). The separated organic layer was washed with brine (10 mL), dried over Na₂SO₄ and evaporated to dryness. The crude product was purified by flash column chromatography on silica gel to give tert-butyl (rel-trans)-3-formyl-4-phenylpyrrolidine-1-carboxylate (300 mg, 1.08 mmol, 60%) as colorless oil. LCMS (ESI): m/z=221.2 [M+H−56]⁺.

### Step B: tert-butyl (rel-trans)-3-(difluoromethyl)-4-phenylpyrrolidine-1-carboxylate

To a solution of tert-butyl (rel-trans)-3-formyl-4-phenylpyrrolidine-1-carboxylate (100 mg, 363 μmol, 1 eq.) in dry DCM (2 mL) was added DAST (117 mg, 726 μmol, 2 eq.) in several portions at 0° C., and the mixture was stirred at room temperature for 14 hrs. The reaction was then quenched with MeOH (2.0 mL), extracted with dichloromethane (10 mL×3). The organic layers were combined and washed with brine (10 mL×2), dried over anhydrous Na₂SO₄ and concentrated under reduced pressure. The residue was purified by column chromatography on silica gel to afford tert-butyl (rel-trans)-3-(difluoromethyl)-4-phenylpyrrolidine-1-carboxylate (100 mg, 336 μmol, 93%) as a colorless oil.

### Step C: (rel-trans)-3-(difluoromethyl)-4-phenylpyrrolidine

To a solution of tert-butyl (rel-trans)-3-(difluoromethyl)-4-phenylpyrrolidine-1-carboxylate (110 mg, 369 μmol, 1 eq.) in DCM (2 mL) was added TFA (419 mg, 3.68 mmol, 10 eq). The reaction mixture was stirred at room temperature for 1 hr. After completion, the reaction was cooled in an ice-bath, then neutralized carefully with NaHCO₃(aq.) until the pH was adjusted to pH=8-9. The resulting mixture was extracted with DCM (10 mL×3), and the combined organic layers were washed with brine (10 mL×2), dried over with anhydrous Na₂SO₄, then concentrated under reduced pressure to give crude (rel-trans)-3-(difluoromethyl)-4-phenylpyrrolidine (73 mg, 369 μmol, quant.) as a white solid, which was used in next step directly without further purification. LCMS (ESI): m/z=198.2 [M+H]⁺.

### Synthesis of 2-(4-amino-[1,1′-biphenyl]-3-yl)acetonitrile

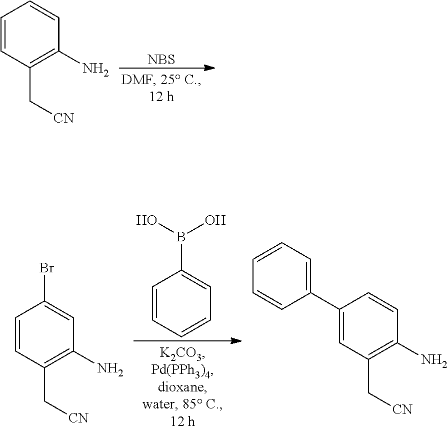

### Step 1: 2-(2-amino-4-bromophenyl)acetonitrile

To a solution of 2-(2-aminophenyl)acetonitrile (2.0 g, 15 mmol, 1 eq) in dimethylformamide (20 mL) was added N-bromosuccinimide (3.0 g, 17 mmol, 1.1 eq). The mixture was stirred at 25° C. for 12 hours. The mixture was diluted with water (200 mL) and extracted with ethyl acetate (200 mL×2), the combined organic layers were washed with saturated brine (200 mL×2), dried over Na₂SO₄, filtered and concentrated under reduced pressure to give 2-(2-amino-4-bromophenyl)acetonitrile (3.2 g, crude) as a white solid. LCMS: m/z (M+H)⁺=210.9.

### Step 2: 2-(4-amino-[1,1′-biphenyl]-3-yl)acetonitrile

To a solution of 2-(2-amino-4-bromophenyl)acetonitrile (1 g, 4.7 mmol, 1 eq) in water (2 mL) and dioxane (8 mL) was added tetrakis(triphenylphosphine) palladium (0.27 g, 0.24 mmol, 0.05 eq), potassium carbonate (1.9 g, 14 mmol, 3 eq) and phenylboronic acid (0.65 g, 5.2 mmol, 1.1 eq). The mixture was degassed and purged with N₂ for 3 times and then stirred at 85° C. for 12 hours. The reaction mixture was filtered and the filtrate was concentrated to give a residue. The residue was extracted with water (100 mL) and ethyl acetate (100 mL×2). The combined organic layers were washed with saturated brine (100 mL×2), dried over Na₂SO₄, filtered and concentrated under reduced pressure to give a residue. The residue was purified by column chromatography (SiO₂, Petroleum ether/Ethyl acetate=1:0 to 2:1) to give 2-(4-amino-[1,1′-biphenyl]-3-yl)acetonitrile (0.50 g, crude) as a white solid. LCMS: m/z (M+1)=209.0.

### Synthesis of 2-(4-amino-[1,1′-biphenyl]-3-yl)acetamide

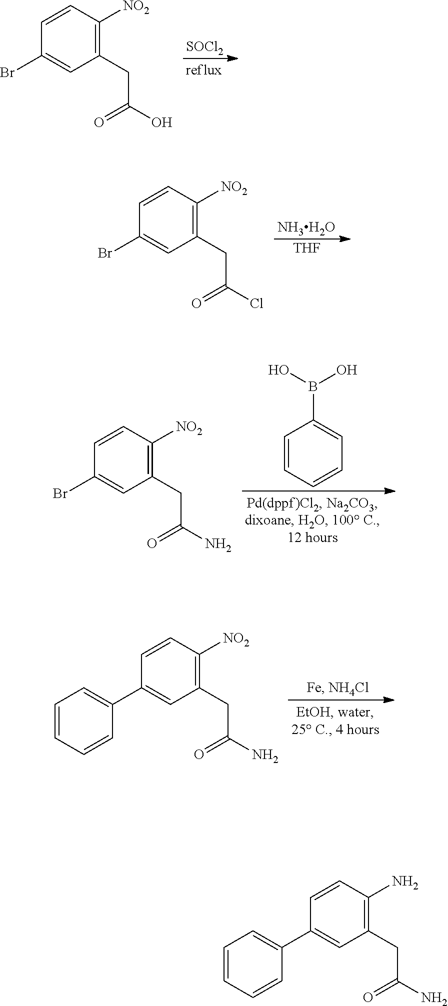

### Step 1: 2-(5-bromo-2-nitrophenyl)acetyl Chloride

A solution of 2-(5-bromo-2-nitrophenyl)acetic acid (0.30 g, 1.2 mmol, 1 eq) in thionyl chloride (2 mL) was stirred at 80° C. for 1 hour. The reaction mixture was concentrated under reduced pressure to give 2-(5-bromo-2-nitrophenyl)acetyl chloride (0.30 g, crude) as a white solid.

### Step 2: 2-(5-bromo-2-nitrophenyl)acetamide

A solution of 2-(5-bromo-2-nitrophenyl)acetyl chloride (0.30 g, 1.1 mmol, 1 eq) in tetrahydrofuran (3 mL) and NH₃·H₂O (4 mL) was stirred at 25° C. for 30 min. The reaction mixture was concentrated under reduced pressure to give 2-(5-bromo-2-nitrophenyl)acetamide (0.30 g, crude) as a white solid. LCMS: m/z (M+H)=258.9.

### Step 3: 2-(4-nitro-[1,1′-biphenyl]-3-yl)acetamide

To a solution of 2-(5-bromo-2-nitrophenyl)acetamide (0.10 g, 0.39 mmol, 1 eq) in dioxane (1 mL) and water (0.2 mL) was added sodium carbonate (0.12 g, 1.2 mmol, 3 eq), phenylboronic acid (52 mg, 0.42 mmol, 1.1 eq) iron(2+) bis(cyclopenta-2,4-diyn-1-yldiphenyl-lambda4-phosphane) palladium dichloride (14 mg, 0.019 mmol, 0.05 eq), the mixture was stirred at 100° C. for 12 hours. The mixture was diluted with water (20 mL) and extracted with ethyl acetate (20 mL×2), the combined organic layers were washed with saturated brine (20 mL×2), dried over Na₂SO₄, filtered and concentrated under reduced pressure to give a residue. The residue was purified by column chromatography to give 2-(4-nitro-[1,1′-biphenyl]-3-yl)acetamide (60 mg, 60% yield) as a white solid. LCMS: m/z (M+23)+=279.0. ¹H NMR (400 MHz, CD₃OD) δ 8.17 (d, J=8.4 Hz, 1H), 7.79-7.69 (m, 4H), 7.52-7.40 (m, 3H), 4.06 (s, 2H)

### Step 4: 2-(4-amino-[1,1′-biphenyl]-3-yl)acetamide

To a solution of 2-(4-nitro-[1,1′-biphenyl]-3-yl)acetamide (60 mg, 0.23 mmol, 1 eq) in ethyl alcohol (0.8 mL) and water (0.2 mL) was added ammonium chloride (0.038 g, 0.70 mmol, 3 eq) and iron (0.13 g, 2.4 mmol, 10 eq). The mixture was stirred at 25° C. for 4 hours. The reaction mixture was filtered and the filtrate was concentrated to give 2-(4-amino-[1,1′-biphenyl]-3-yl)acetamide (0.043 g, crude) as a white solid. LCMS: m/z (M+H)=227.0. ¹H NMR (400 MHz, DMSO) δ 7.53 (d, J=7.6 Hz, 3H), 7.41-7.35 (m, 3H), 7.30-7.20 (m, 2H), 6.98 (s, 1H), 6.73 (d, J=8.4 Hz, 1H), 5.26 (s, 2H), 3.32 (s, 2H).

### Synthesis of 3-(thiazol-2-ylmethyl)-[1,1′-biphenyl]-4-amine

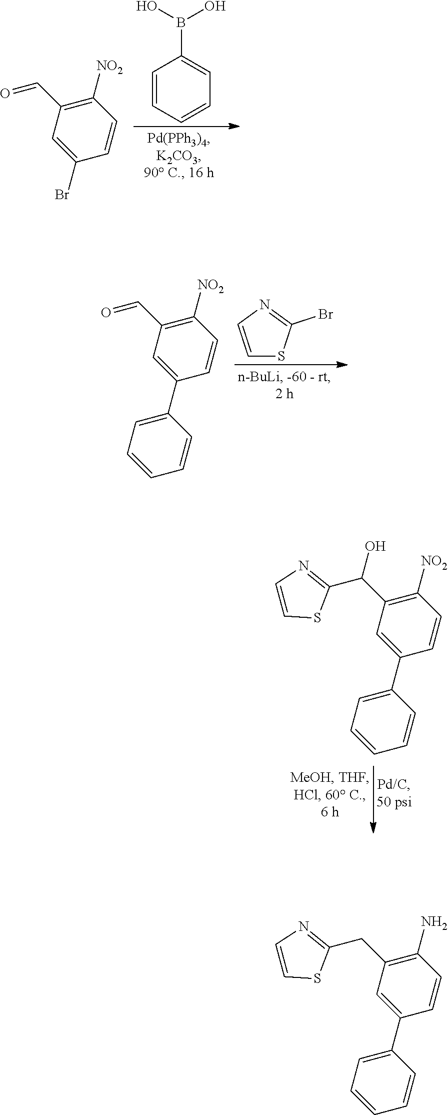

### Step 1: 4-nitro-[1,1′-biphenyl]-3-carbaldehyde

To a solution of 5-bromo-2-nitrobenzaldehyde (5 g, 22 mmol, 1 eq) in dioxane (30 mL) and water (10 mL) was added phenylboronic acid (5.3 g, 43 mmol, 2 eq) and potassium carbonate (12 g, 87 mmol, 4 eq), tetrakis(triphenylphosphine) palladium (1.2 g, 1.1 mmol, 0.05 eq) under N₂. The reaction was stirred at 90° C. for 16 hours to give a black solution. The reaction mixture was diluted with water (50 mL) and extracted with ethyl acetate (50 mL×2), the combined organic layers were washed with saturated brine (50 mL×2), dried over Na₂SO₄, filtered and concentrated under reduced pressure to give a residue. The residue was purified by flash silica gel chromatography to give 4-nitro-[1,1′-biphenyl]-3-carbaldehyde (3.5 g, 71% yield) as a yellow solid. ¹H NMR (400 MHz, CHLOROFORM-d) δ 10.52 (s, 1H), 8.22 (d, J=8.4 Hz, 1H), 8.14 (d, J=2.0 Hz, 1H), 7.94 (d, J=6.4 Hz, 1H), 7.69-7.63 (m, 2H), 7.56-7.46 (m, 3H)

### Step 2: (4-nitro-[1,1′-biphenyl]-3-yl)(thiazol-2-yl)methanol

To a solution of 2-bromo-1,3-thiazole (2.2 g, 13 mmol, 1 eq) in tetrahydrofuran (30 mL) at −60° C. was added dropwise monobutyl lithium hydride (6.3 mL, 16 mmol, 1.2 eq) and stirred for 30 mins at −60° C. Then the solution of 4-nitro-[1,1′-biphenyl]-3-carbaldehyde (3 g, 13 mmol, 1 eq) in THF (10 mL) was added dropwise at −60° C. The reaction mixture was warmed to 25° C. and stirred for 2 hours to give a black solution. The reaction mixture was quenched with saturated ammonium chloride (80 mL). The mixture was diluted with water (150 mL) and extracted with ethyl acetate (150 mL×2), the combined organic layers were washed with saturated brine (150 mL×2), dried over Na₂SO₄, filtered and concentrated under reduced pressure to give a residue. The residue was purified by flash silica gel chromatography to give (4-nitro-[1,1′-biphenyl]-3-yl)(thiazol-2-yl)methanol (1.9 g, 46% yield) as a yellow solid. ¹H NMR (400 MHz, CHLOROFORM-d) δ 8.17 (d, J=8.4 Hz, 1H), 8.00 (d, J=1.6 Hz, 1H), 7.79-7.67 (m, 2H), 7.67-7.57 (m, 2H), 7.53-7.42 (m, 3H), 7.35 (d, J=3.2 Hz, 1H), 6.76 (s, 1H)

### Step 3: 3-(thiazol-2-ylmethyl)-[1,1′-biphenyl]-4-amine

To a solution of (4-nitro-[1,1′-biphenyl]-3-yl)(thiazol-2-yl)methanol (0.1 g, 0.3 mmol, 1 eq) in methanol (1 mL) and tetrahydrofuran (1 mL) was added HCl (0.5 mL, 1 M) and Pd/C (0.1 g) under H₂. The mixture was stirred at 60° C. for 6 hours at 50 Psi. The reaction mixture was filtered and the filtrate was concentrated to give a residue. The residue was purified by flash silica gel chromatography to give 3-(thiazol-2-ylmethyl)-[1,1′-biphenyl]-4-amine (70 mg, 82% yield) as a red oil. LCMS: (ES+) m/z (M+H)⁺=266.9 ¹H NMR (400 MHz, CHLOROFORM-d) δ 7.60 (d, J=3.2 Hz, 1H), 7.47 (dd, J=1.2, 8.4 Hz, 2H), 7.36-7.26 (m, 4H), 7.22-7.17 (m, 1H), 7.12 (d, J=3.2 Hz, 1H), 6.69 (d, J=8.0 Hz, 1H), 4.24 (s, 2H).

### Synthesis of 6-phenyl-1,2,3,4-tetrahydroquinoline-4-carboxamide

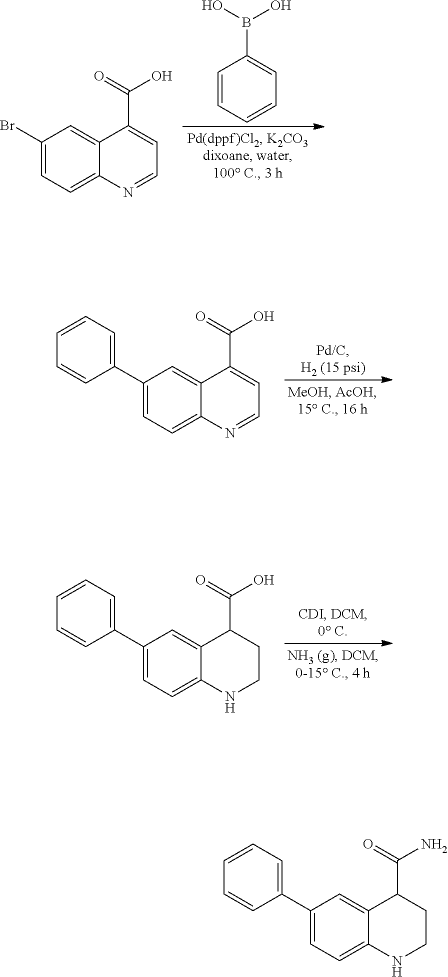 [SMILES: O=C(O)c1ccnc2ccc(Br)cc12 ; OB(O)c1ccccc1 ; O=C(O)c1ccnc2ccc(-c3ccccc3)cc12 ; O=C(O)C1CCNc2ccc(-c3ccccc3)cc21 ; NC(=O)C1CCNc2ccc(-c3ccccc3)cc21]

### Step 1: 6-phenylquinoline-4-carboxylic Acid

To a solution of 6-bromoquinoline-4-carboxylic acid (0.50 g, 2.0 mmol) and phenylboronic acid (0.29 g, 2.4 mmol), palladium(2+) bis(triphenylphosphine) dichloride (0.14 g, 0.20 mmol) in dioxane (6 mL) and water (2 mL) was added potassium carbonate (0.55 g, 4.0 mmol). Then the reaction mixture was stirred at 100° C. for 3 hours to give a black suspension. The reaction mixture was filtered and the filtrate was collected, poured into water (20 mL) and adjust to pH=12 with NaOH (2 M) and washed with EtOAc (30 mL×2), then aqueous phase was adjust to pH=5 with HCl (1 M) to give a white suspension. The suspension was extracted with ethyl acetate (30 mL×2). The combined organic phase was washed with sat. brine (30 mL), dried over anhydrous Na₂SO₄ and then filtered, the filtration was concentrated in vacuum to give 6-phenylquinoline-4-carboxylic acid (0.6 g, crude) as a white solid. LCMS: m/z (M+H)⁺=251.0

### Step 2: ethyl 5-iodobenzo[d]thiazole-2-carboxylate

6-phenylquinoline-4-carboxylic acid (1.2 g, 4.8 mmol) was dissolved in AcOH (5 mL) and MeOH (10 mL) under N₂, then Pd/C (0.15 g, dry) was added at 15° C. under N₂, then the reaction mixture was stirred at 15° C. for 16 hours under H₂ (15 psi) to give a black suspension. The reaction mixture was filtered and the filtrate was concentrated to give a residue. The residue was purified by silica gel chromatography to give 6-phenyl-1,2,3,4-tetrahydroquinoline-4-carboxylic acid (0.45 g, 37% yield) as a white solid. LCMS: m/z (M+H)+=254.0

### Step 3: 6-phenyl-1,2,3,4-tetrahydroquinoline-4-carboxamide

6-phenyl-1,2,3,4-tetrahydroquinoline-4-carboxylic acid (0.3 g, 1.2 mmol) was dissolved in DCM (5 mL) was added carbonyldiimidazole (0.21 g, 1.3 mmol) at 0° C. for 30 min, then NH₃ (15 psi, balloon) was added and then the reaction mixture was stirred at 15° C. for 4 hours to give a yellow solution. The mixture was poured into water (35 mL), the aqueous phase was extracted with ethyl acetate (35 mL×3). The combined organic phase was washed with brine (35 mL×2), dried with anhydrous Na₂SO₄, filtered and concentrated in vacuum to give a residue. The residue was purified by silica gel chromatography to give 6-phenyl-1,2,3,4-tetrahydroquinoline-4-carboxamide (0.15 g, 50% yield) as a yellow oil. ¹H NMR (400 MHz, CHLOROFORM-d) δ 7.55-7.50 (m, 2H), 7.44-7.34 (m, 3H), 7.34-7.28 (m, 2H), 6.64 (d, J=8.4 Hz, 1H), 5.82-5.38 (m, 2H), 3.81-3.69 (m, 1H), 3.44-3.28 (m, 2H), 2.52 (qd, J=3.2, 13.2 Hz, 1H), 2.11-2.00 (m, 1H).

### Synthesis of 2-(4-amino-[1,1′-biphenyl]-3-yl)-N-methylacetamide

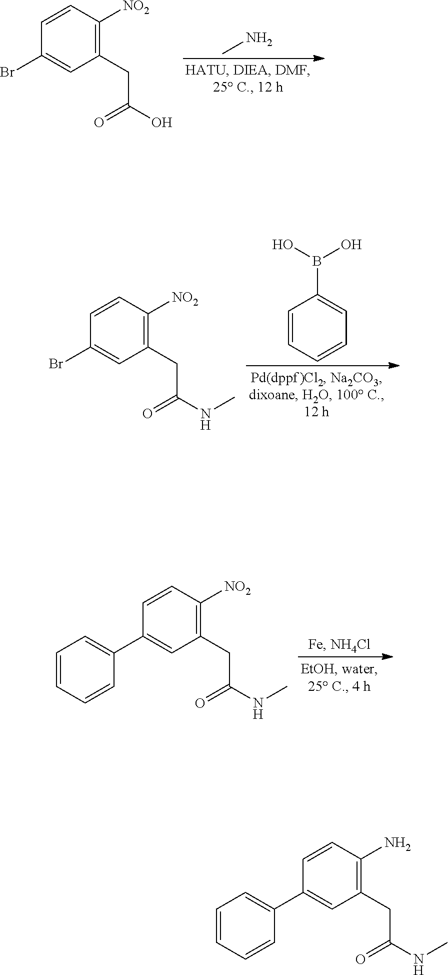

### Step 1: 2-(5-bromo-2-nitrophenyl)-N-methylacetamide

To a solution of 2-(5-bromo-2-nitrophenyl)acetic acid (0.10 g, 0.38 mmol, 1 eq) in methylene chloride (1 mL) was added O-(7-azabenzotriazol-1-yl)-N,N,N,N-tetramethyluroniumhexafluorophosphate (0.062 g, 0.38 mmol, 1 eq) and N,N-diisopropylethylamine (0.13 g, 1.5 mmol, 3 eq), the mixture was stirred at 25° C. for 15 min. Then the mixture was added methanamine (0.014 g, 0.46 mmol, 1.2 eq). The mixture was stirred at 25° C. for 12 hours. The mixture was purified by column chromatography (SiO₂, Petroleum ether/Ethyl acetate=1:0 to 0:1) and concentrated in vacuo to give 2-(5-bromo-2-nitrophenyl)-N-methylacetamide (0.10 g, 96.1% yield) as a white solid. LCMS: m/z (M+H)⁺=272.8. ¹H NMR (400 MHz, METHANOL-d₄) δ7.98 (d, J=9.2 Hz, 1H), 7.73-7.63 (m, 2H), 3.91 (s, 2H), 2.73 (s, 3H).

### Step 2: N-methyl-2-(4-nitro-[1,1′-biphenyl]-3-yl)acetamide

To a solution of 2-(5-bromo-2-nitrophenyl)-N-methylacetamide (0.10 g, 0.37 mmol, 1 eq) in dioxane (1 mL) and water (0.2 mL) was added sodium carbonate (0.12 g, 1.1 mmol, 3 eq), phenylboronic acid (0.054 g, 0.44 mmol, 1.2 eq) and iron(2+) bis(cyclopenta-2,4-diyn-1-yldiphenyllambda4-phosphane) palladium dichloride (0.013 g, 0.018 mmol, 0.05 eq), the mixture was stirred at 100° C. for 12 hours. The reaction mixture was filtered to give a filtrate. The filtrate was diluted with water (20 mL) and extracted with ethyl acetate (20 mL×2), the combined organic layers were washed with saturated brine (20 mL×2), dried over Na₂SO₄, filtered and concentrated under reduced pressure to give a residue. The residue was purified by column chromatography (SiO₂, Petroleum ether/Ethyl acetate=10:1 to 0:1) to give N-methyl-2-(4-nitro-[1,1′-biphenyl]-3-yl)acetamide (0.10 g, 91.0% yield) as a white solid. LCMS: m/z (M+H)⁺=271.0

### Step 3: 2-(4-amino-[1,1′-biphenyl]-3-yl)-N-methylacetamide

To a solution of N-methyl-2-(4-nitro-[1,1′-biphenyl]-3-yl)acetamide (0.10 g, 0.37 mmol, 1 eq) in ethyl alcohol (0.8 mL) and water (0.2 mL) was added ammonium chloride (0.059 g, 1.1 mmol, 3 eq) and iron (0.21 g, 3.7 mmol, 10 eq). The mixture was stirred at 25° C. for 4 hours. The reaction mixture was filtered and the filtrate was concentrated to give 2-(4-amino-[1,1′-biphenyl]-3-yl)-N-methylacetamide (0.10 g, crude) as a white solid. LCMS: m/z (M+H)⁺=241.2.

### Synthesis of (rel-trans)-2-phenylazetidine-3-carbonitrile

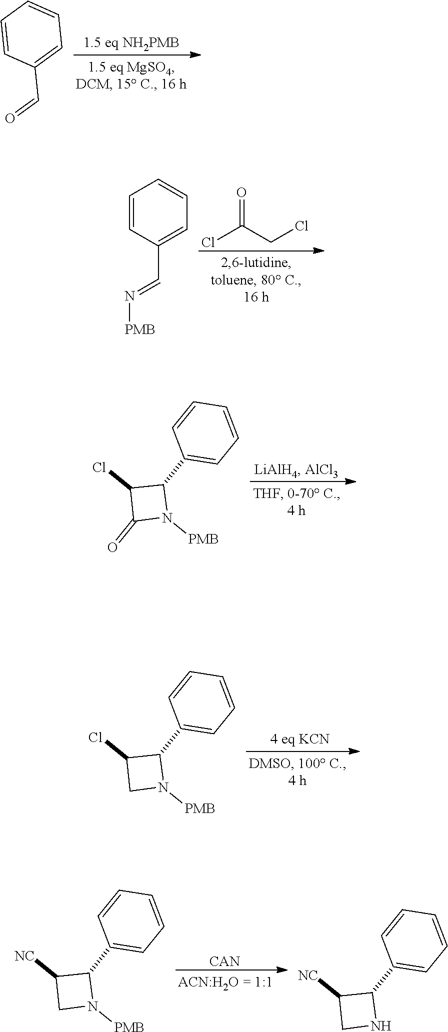

### Step 1: (E)-N-benzylidene-1-(4-methoxyphenyl)methanamine

To a solution of benzaldehyde (5.0 g, 36 mmol, 1.5 eq) and 1-(4-methoxyphenyl)methanamine (9.7 g, 71 mmol, 1.5 eq) in DCM (15 mL) was added magnesium sulfate (4.4 g, 36 mmol, 1.5 eq) at 15° C., then the reaction mixture was stirred at 15° C. for 16 hours to give a white suspension. The mixture was poured into water (50 mL). The aqueous phase was extracted with ethyl acetate (50 mL×2). The combined organic phase was washed with brine (50 mL×2), dried with anhydrous Na₂SO₄, filtered and concentrated in vacuum to give a residue. The residue was purified by silica gel chromatography to afford (E)-N-benzylidene-1-(4-methoxyphenyl)methanamine (5.6 g, 82% yield) as a yellow oil. ¹H NMR (400 MHz, CHLOROFORM-d) δ 8.27 (s, 1H), 7.71-7.63 (m, 2H), 7.46-7.28 (m, 3H), 7.16 (d, J=8.8 Hz, 2H), 6.83-6.75 (m, 2H), 4.67 (s, 2H), 3.69 (s, 3H).

### Step 2: (rel-trans)-3-chloro-1-(4-methoxybenzyl)-4-phenylazetidin-2-one

To a solution of (E)-N-benzylidene-1-(4-methoxyphenyl)methanamine (5.6 g, 25 mmol, 1 eq) and lutidine (6.7 g, 62 mmol, 2.5 eq) in toluene (30 mL) was added 2-chloroacetyl chloride (4.2 g, 37 mmol, 1.5 eq) at 15° C., then the reaction mixture was stirred at 80° C. for 16 hours to give a yellow solution. The mixture was diluted with water (100 mL) and extracted with ethyl acetate (100 mL×2), the combined organic layers were washed with saturated brine (100 mL×2), dried over Na₂SO₄, filtered and concentrated under reduced pressure to give a residue. The residue was purified by silica gel chromatography to afford (rel-trans)-3-chloro-1-(4-methoxybenzyl)-4-phenylazetidin-2-one (4.5 g, 60% yield) as a yellow oil. LCMS: m/z (M+H)⁺=302.1. ¹H NMR (400 MHz, CHLOROFORM-d) δ 7.50-7.39 (m, 3H), 7.27 (s, 2H), 7.11 (s, 2H), 6.89-6.82 (m, 2H), 4.89-4.81 (m, 1H), 4.57 (d, J=0.8 Hz, 1H), 4.38 (d, J=1.2 Hz, 1H), 3.83 (s, 3H), 3.80 (d, J=15.2 Hz, 1H).

### Step 3: (rel-trans)-3-chloro-1-(4-methoxybenzyl)-2-phenylazetidine

To a solution of aluminium chloride (5.9 g, 4.9 mmol, 3 eq) in THF (45 mL) was added aluminium(3+) lithium(1+) tetrahydride (1.7 g, 4.9 mmol, 3 eq) at 0° C., then the reaction mixture was stirred at 0° C. for 10 min, and stirred at 70° C. for 30 min. (rel-trans)-3-chloro-1-(4-methoxybenzyl)-4-phenylazetidin-2-one (4.5 g, 1.7 mmol, 1 eq) in THF (45 mL) was added slowly, the reaction was stirred at 70° C. for 4 hours to give white suspension. 5 mL water was added to the reaction at 0° C. and stirred for 30 min, then the reaction mixture was filtered and the filtrate was collected and concentrated in vacuum to give a yellow solid. The residue was purified by silica gel chromatography to give (rel-trans)-3-chloro-1-(4-methoxybenzyl)-2-phenylazetidine (2.5 g, 59% yield) as a off-yellow oil. LCMS: m/z (M+H)⁺=288.1.

### Step 4: (rel-trans)-1-(4-methoxybenzyl)-2-phenylazetidine-3-carbonitrile

To a solution of (rel-trans)-3-chloro-1-(4-methoxybenzyl)-2-phenylazetidine (1.5 g, 5.2 mmol, 1 eq) in dimethyl sulfoxide (20 mL) was added potassium cyanide (0.67 g, 10 mmol, 2 eq), the mixture was stirred at 100° C. for 4 hours to give a brown solution. The mixture was diluted with water (100 mL) and extracted with ethyl acetate (100 mL×2), the combined organic layers were washed with saturated brine (100 mL×2), dried over Na₂SO₄, filtered and concentrated under reduced pressure to give a residue. The aqueous phase was adjust to pH=14 with 1N NaOH, then NaOCl.aq (200 mL) was added and place for 48 hours, wait for the professional testing personnel pass then discard. The residue was purified by column chromatography (SiO₂, Petroleum ether/Ethyl acetate=100:1 to 90:10) to give (rel-trans)-1-(4-methoxybenzyl)-2-phenylazetidine-3-carbonitrile (0.65 g, 45% yield) as a yellow oil. ¹H NMR (400 MHz, DMSO-d₆) δ 7.49-7.44 (m, 2H), 7.41-7.30 (m, 3H), 7.20-7.15 (m, 2H), 6.86-6.81 (m, 2H), 4.31 (d, J=8.4 Hz, 1H), 3.71 (s, 3H), 3.66 (d, J=12.8 Hz, 1H), 3.49-3.42 (m, 2H), 3.40-3.34 (m, 1H), 3.10-3.04 (m, 1H).

### Step 5: (rel-trans)-2-phenylazetidine-3-carbonitrile

To a solution of (rel-trans)-1-(4-methoxybenzyl)-2-phenylazetidine-3-carbonitrile (0.1 g, 0.36 mmol, 1 eq) in acetonitrile (3 mL) and water (3 mL) was added cerium(4+) bis(nitric acid) diamine tetranitrate (1.2 g, 2.2 mmol, 6 eq) to give a red solution, the mixture was stirred at 60° C. for 12 hours to give a yellow solution. The mixture was diluted with water (50 mL) and extracted with ethyl acetate (50 mL×2), the combined organic layers were washed with saturated brine (50 mL×2), dried over Na₂SO₄, filtered and concentrated under reduced pressure to give a residue. The residue was purified by column chromatography (SiO₂, DCM:MeOH=100:1 to 10:1, UV=220 nm) to give a (rel-trans)-2-phenylazetidine-3-carbonitrile (80 mg, crude) as a yellow oil. LCMS: m/z (M+H)⁺=159.6.

### Synthesis of (3R,5R)-5-phenylpyrrolidine-3-carbonitrile and (3R,5S)-5-phenylpyrrolidine-3-carbonitrile

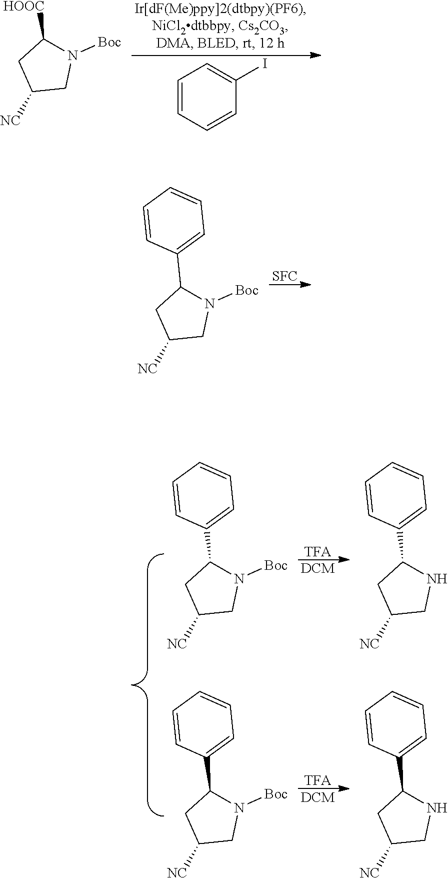

### Step 1: (4R)-tert-butyl 4-cyano-2-phenylpyrrolidine-1-carboxylate

An oven-dried 40 mL vial equipped with magnetic stir bar was charged with iodobenzene (0.20 g, 0.98 mmol, 1 eq), (3R,4R)-1-[(tert-butoxy)carbonyl]-4-cyanopyrrolidine-3-carboxylic acid (0.31 g, 1.3 mmol, 1.3 eq), Ir[dF(Me)ppy]₂(dtbpy)(PF₆) (8.0 mg, 0.01 eq), NiCl₂·dtbbpy (12 mg, 0.05 eq), and caesium carbonate (0.48 g, 1.5 mmol, 1.5 eq) in DMA (10 mL). The vial was placed under nitrogen and sealed. The reaction was stirred and irradiated with a 10 W blue LED lamp (3 cm away), with cooling water to keep the reaction temperature at 25° C. for 12 hours. The mixture was purified by prep-HPLC (column: Phenomenex Luna C18 150×25 mm×10 um, mobile phase: water (0.1% TFA)-ACN; B %: 38%-68%, 11.5 min) to lyophilization to give (4R)-tert-butyl 4-cyano-2-phenylpyrrolidine-1-carboxylate (0.15 g, 55% yield) as a yellow oil. ¹H NMR (400 MHz, CHLOROFORM-d) δ 7.30-7.23 (m, 2H), 7.22-7.13 (m, 2H), 7.06 (d, J=7.2 Hz, 1H), 5.14-4.60 (m, 1H), 3.90-3.80 (m, 1H), 3.15-3.01 (m, 1H), 2.80-2.69 (m, 1H), 2.65-2.48 (m, 1H), 2.21-2.13 (m, 1H), 1.57-0.93 (m, 9H)

### Step 2: (2R,4R)-tert-butyl 4-cyano-2-phenylpyrrolidine-1-carboxylate and (2S,4R)-tert-butyl 4-cyano-2-phenylpyrrolidine-1-carboxylate SFC Separation

SFC showed (4R)-tert-butyl 4-cyano-2-phenylpyrrolidine-1-carboxylate have two peaks (rt=0.848 1.136 min). The residue was purified by SFC (column: REGIS(S,S)WHELK-01 (250 mm×25 mm, 10 um), mobile phase: 0.1% NH₃H₂O MeOH; B %: 15%-15%) and concentrated under reduced pressure to give (2R,4R)-tert-butyl 4-cyano-2-phenylpyrrolidine-1-carboxylate (40 mg, 27% yield, rt=0.848 min) as a white solid and (2S,4R)-tert-butyl 4-cyano-2-phenylpyrrolidine-1-carboxylate (80 mg, 54% yield, rt=1.136 min) as a white solid. (2R,4R)-tert-butyl 4-cyano-2-phenylpyrrolidine-1-carboxylate: ¹H NMR (400 MHz, CHLOROFORM-d) δ 7.38-7.31 (m, 2H), 7.30-7.19 (m, 3H), 4.85-4.52 (m, 1H), 4.20 (s, 1H), 3.78-3.69 (m, 1H), 3.17-3.03 (m, 1H), 2.87-2.76 (m, 1H), 2.28-2.11 (m, 1H), 1.51-1.01 (m, 9H) (2S,4R)-tert-butyl 4-cyano-2-phenylpyrrolidine-1-carboxylate: ¹H NMR (400 MHz, CHLOROFORM-d) δ 7.30-7.22 (m, 2H), 7.22-7.15 (m, 1H), 7.06 (d, J=7.2 Hz, 2H), 5.18-4.84 (m, 1H), 3.91-3.64 (m, 2H), 3.08 (m, 1H), 2.56 (m, 1H), 2.24-2.07 (m, 1H), 1.43-1.03 (m, 9H)

### Step 3: (3R,5R)-5-phenylpyrrolidine-3-carbonitrile and (3R,5S)-5-phenylpyrrolidine-3-carbonitrile

To a solution of tert-butyl (4R)-4-cyano-2-phenylpyrrolidine-1-carboxylate (1 eq) in methylene chloride (0.1 mL) and trifluoroacetic acid (0.3 mL), the mixture was stirred at 25° C. for 1 hour to give a yellow solution. The reaction mixture was concentrated under reduced pressure to give (3R,5R)-5-phenylpyrrolidine-3-carbonitrile (40 mg, crude) and (3R,5S)-5-phenylpyrrolidine-3-carbonitrile (80 mg, crude) as a yellow oil. (3R,5R)-5-phenylpyrrolidine-3-carbonitrile: LCMS: m/z (M+H)⁺=173.0 (3R,5S)-5-phenylpyrrolidine-3-carbonitrile: LCMS: m/z (M+H)⁺=173.0

### Synthesis of (rel-trans)-2-phenylpyrrolidine-3-carbonitrile

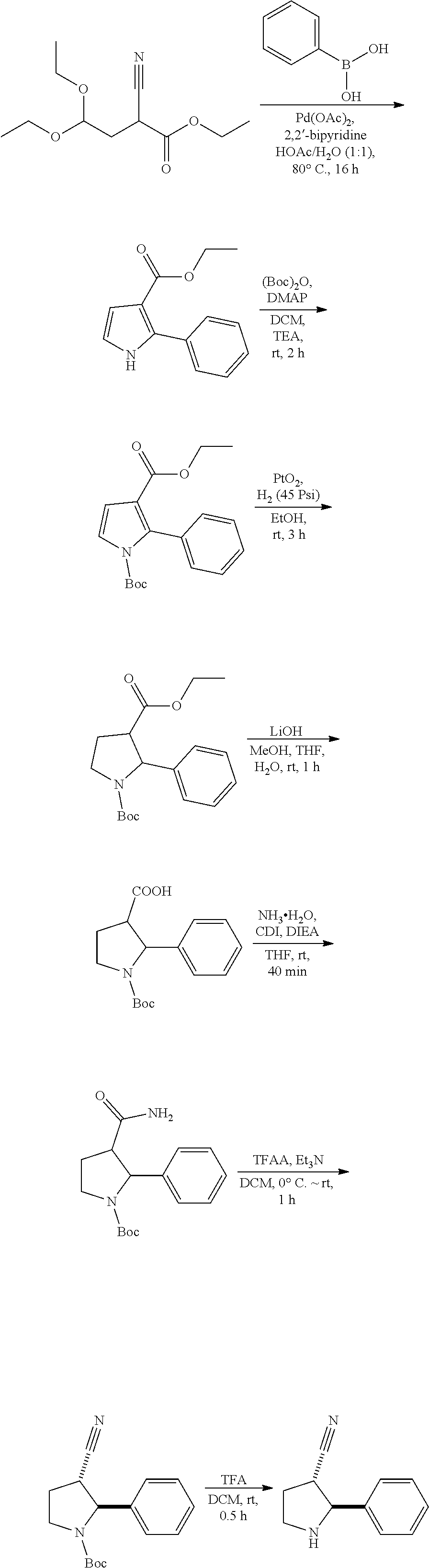

### Step 1: ethyl 2-(cyclohexa-1,5-dien-1-yl)-1H-pyrrole-3-carboxylate

To a solution of ethyl 2-cyano-4,4-diethoxybutanoate (8.0 g, 35 mmol, 1.0 eq) in acetic acid (12 mL) and water (12 mL) was added phenylboronic acid (5.1 g, 42 mmol, 1.2 eq), bipyridyl (0.27 g, 1.7 mmol, 0.05 eq), the mixture was purged with N₂ for 3 times. Then the mixture added palladium(2+) acetate (0.29 g, 1.7 mmol, 0.05 eq) under N₂ atmosphere and was stirred at 80° C. for 16 hours to give a black muddy solution. The reaction was diluted with water (500 mL) and extracted with ethyl acetate (500 mL×2), the combined organic layers were washed with saturated. brine (500 mL×2), dried over Na₂SO₄, filtered and concentrated under reduced pressure to give a residue. The residue was purified by flash silica gel chromatography (80 g SepaFlash® Silica Flash Column, Eluent of 0˜10% Ethyl acetate/Petroleum ether gradient @80 mL/min) to give ethyl 2-(cyclohexa-1,5-dien-1-yl)-1H-pyrrole-3-carboxylate (3.00 g, 40% yield) as a white solid. LCMS: m/z (M+H)⁺=215.9. 1H NMR (400 MHz, CHLOROFORM-d) δ 8.54 (s, 1H), 7.66-7.55 (m, 2H), 7.45-7.32 (m, 3H), 6.75 (d, J=2.4 Hz, 2H), 4.21 (q, J=7.2 Hz, 2H), 1.26 (t, J=7.2 Hz, 3H).

### Step 2: 1-tert-butyl 3-ethyl 2-phenyl-1H-pyrrole-1,3-dicarboxylate

To a solution of ethyl 2-(cyclohexa-1,5-dien-1-yl)-1H-pyrrole-3-carboxylate (3.0 g, 14 mmol, 1.0 eq) in DCM (30 mL) was added di-tert-butyl dicarbonate (9.1 g, 42 mmol, 3.0 eq), 4-dimethylaminopyridine (0.17 g, 1.4 mmol, 0.1 eq) and triethylamine (2.1 g, 21 mmol, 1.5 eq), the mixture was stirred at 25° C. for 1 hour to give a black clean solution. The reaction was diluted with water (200 mL) and extracted with ethyl acetate (200 mL×2), the combined organic layers were washed with saturated brine (200 mL×2), dried over Na₂SO₄, filtered and concentrated under reduced pressure to give a residue. The residue was purified by flash silica gel chromatography to give 1-tert-butyl 3-ethyl 2-phenyl-1H-pyrrole-1,3-dicarboxylate (3.5 g, 80% yield) as a light-yellow oil. LCMS: m/z (M+H)⁺=316.0. ¹H NMR (400 MHz, CHLOROFORM-d) δ 7.32-7.27 (m, 3H), 7.25 (dd, J=1.2, 3.6 Hz, 1H), 7.23-7.17 (m, 2H), 6.60 (dd, J=1.2, 3.6 Hz, 1H), 4.00 (q, J=7.2 Hz, 2H), 1.16 (s, 9H), 1.00 (t, J=7.2 Hz, 3H).

### Step 3: 1-tert-butyl 3-ethyl 2-phenylpyrrolidine-1,3-dicarboxylate

To a solution tert-butyl 3-ethyl 2-phenyl-1H-pyrrole-1,3-dicarboxylate (3.5 g, 11 mmol, 1.0 eq) in EtOH (30 mL) was added dioxoplatinum (7.5 g, 33 mmol, 3.0 eq). The mixture was stirred at 25° C. under H₂ for 3 hours to give a colorless solution. The reaction mixture was filtered, and the filtrate was concentrated to give 1-tert-butyl 3-ethyl 2-phenylpyrrolidine-1,3-dicarboxylate (1.8 g, crude) as a white solid. LCMS: m/z (M-Boc+H)⁺=219.9. ¹H NMR (400 MHz, CHLOROFORM-d) δ=7.26-7.19 (m, 3H), 7.16-7.09 (m, 2H), 5.27-4.99 (m, 1H), 3.95-3.73 (m, 3H), 3.57-3.37 (m, 2H), 2.56-2.35 (m, 1H), 2.13-2.01 (m, 1H), 1.57 (s, 9H), 1.17 (s, 3H).

### Step 4: 1-(tert-butoxycarbonyl)-2-phenylpyrrolidine-3-carboxylic Acid

To a solution of 1-tert-butyl 3-ethyl 2-phenylpyrrolidine-1,3-dicarboxylate (1.8 g, 5.6 mmol, 1.0 eq) in THF (6.0 mL), water (6.0 mL) and MeOH (6.0 mL) was added lithium(1+) hydrate hydroxide (0.47 g, 11 mmol, 2.0 eq), The mixture was stirred at 25° C. for 1 hour to give a yellow clean solution. The reaction mixture was filtered and the filtrate was concentrated to give a residue. The residue was purified by prep-HPLC (basic condition; (column: Welch Xtimate C18 150×25 mm×5 um, mobile phase: water(NH₃H₂O)-ACN; B %:B %:5%-35%, 8 min) to freeze-drying to give 1-(tert-butoxycarbonyl)-2-phenylpyrrolidine-3-carboxylic acid (0.8 g, 2.7 mmol, 49% yield) as a white solid. LCMS: m/z (M−H)⁻=290.1

### Step 5: tert-butyl 3-carbamoyl-2-phenylpyrrolidine-1-carboxylate

To a solution 1-(tert-butoxycarbonyl)-2-phenylpyrrolidine-3-carboxylic acid (0.8 g, 2.7 mmol, 1.0 eq) in THF (10 mL) was added carbonyldiimidazole (1.3 g, 8.2 mmol, 3 eq). The mixture was stirred at 25° C. for 30 minutes to give a colorless clean solution. Then NH₃·H₂O (3.0 mL) was added and the reaction mixture was stirred for 10 minutes to give a colorless clean solution. The reaction mixture was concentrated under reduced pressure to give a residue. The residue was purified by flash silica gel chromatography (12 g SepaFlash® Silica Flash Column, Eluent of 0-10% DCM:MeOH @22 mL/min) to give tert-butyl 3-carbamoyl-2-phenylpyrrolidine-1-carboxylate (0.8 g, 2.1 mmol, 75% yield) as a white solid. LCMS: m/z (M-Boc+H)⁺=191.1. ¹H NMR (400 MHz, CHLOROFORM-d) δ 7.32-7.11 (m, 5H), 5.70 (s, 1H), 5.43 (s, 1H), 4.87 (s, 1H), 3.88 (s, 1H), 3.63-3.52 (m, 1H), 2.78 (d, J=6.8 Hz, 1H), 2.26-2.07 (m, 2H), 1.45-1.05 (m, 9H).

### Step 6: (rel-trans)-tert-butyl 3-cyano-2-phenylpyrrolidine-1-carboxylate

To a solution tert-butyl 3-carbamoyl-2-phenylpyrrolidine-1-carboxylate (0.6 g, 2.1 mmol, 1.0 eq) in DCM (6.0 mL) was added trimethylamine (0.5 g, 5.2 mmol, 2.5 eq), the mixture was cooled to 0° C. and was added trifluoroacetic anhydride (0.5 g, 2.6 mmol, 1.3 eq). The reaction was allowed to warm to 25° C. and was stirred for 1 hour to give a colorless solution. The reaction mixture was concentrated under reduced pressure to give a residue. The residue was purified by prep-HPLC (FA condition; (column: Phenomenex Luna C18 100×30 mm×5 um, mobile phase:water(FA)-ACN; B %:B %: 42%-72%, 8 min) to lyophilization to (2S,3S)-tert-butyl 3-cyano-2-phenylpyrrolidine-1-carboxylate (0.2 g, 0.73 mmol, 36% yield) as a white solid. Rel-trans stereochemistry assigned by 2D NMR. LCMS: m/z (M-t-bu+H)⁺=217.1. 1H NMR (400 MHz, CHLOROFORM-d) δ 7.40-7.28 (m, 3H), 7.20 (d, J=7.2 Hz, 2H), 5.02 (s, 1H), 3.87 (s, 1H), 3.76-3.68 (m, 1H), 3.05-2.93 (m, 1H), 2.36-2.16 (m, 2H), 1.49-1.13 (m, 9H)

### Step 7: (rel-trans)-2-phenylpyrrolidine-3-carbonitrile

(rel-trans)-tert-butyl 3-cyano-2-phenylpyrrolidine-1-carboxylate (50 mg, 0.18 mmol, 1.0 eq) was taken up in a solution of DCM (1.0 mL) and TFA (0.5 mL). The reaction was stirred at 25° C. for 0.5 hours to give a colorless solution. The reaction mixture was concentrated under reduced pressure to give (2S,3S)-2-phenylpyrrolidine-3-carbonitrile (50 mg, crude) as a light-yellow oil and was used in the next step without further purification.

### Synthesis of (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylic Acid

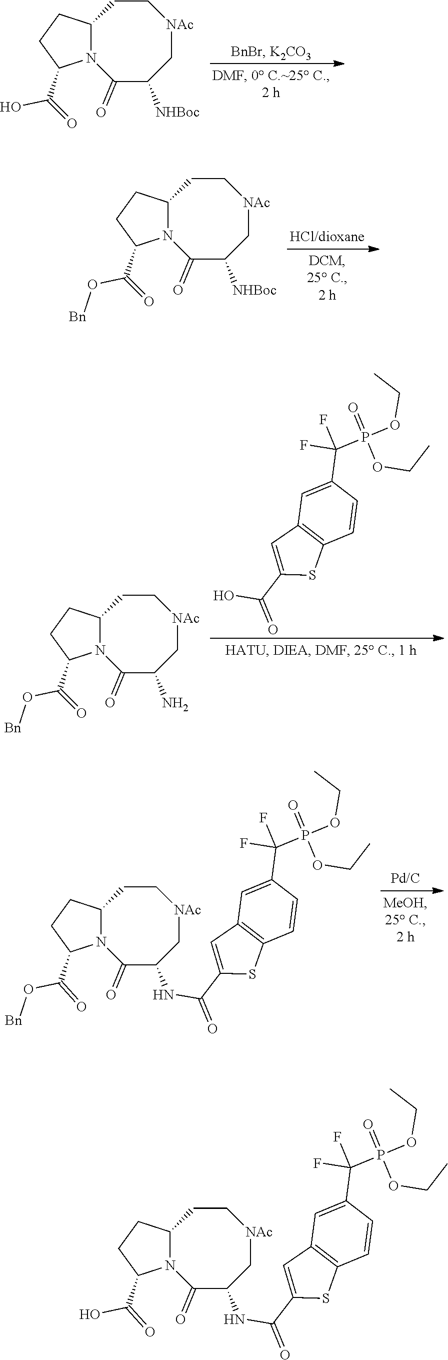

### Step 1: (5S,8S,10aR)-benzyl 3-acetyl-5-((tert-butoxycarbonyl)amino)-6-oxodecahydropyrrolo[1,2-a][1,5]diazocine-8-carboxylate

To a solution of (5S,8S,10aR)-3-acetyl-5-{[(tert-butoxy)carbonyl]amino}-6-oxo-decahydropyrrolo[1,2-a][1,5]diazocine-8-carboxylic acid (1.0 g, 2.7 mmol, 1.0 eq) in dimethylformamide (10 mL) was added K₂CO₃ (0.56 g, 4.1 mmol, 1.5 eq), then (bromomethyl)benzene (0.5 g, 3.0 mmol, 1.1 eq) was added at 0° C. The mixture was stirred at 25° C. for 2 hours. The reaction was diluted with water (100 mL) and extracted with ethyl acetate (100 mL×2), the combined organic layers were washed with saturated brine (100 mL×2), dried over Na₂SO₄, filtered and concentrated under reduced pressure to give a residue. The residue was purified by flash silica gel chromatography to give benzyl (5S,8S,10aR)-benzyl 3-acetyl-5-((tert-butoxycarbonyl)amino)-6-oxodecahydropyrrolo[1,2-a][1,5]diazocine-8-carboxylate (0.5 g, 40% yield) as a white solid. LCMS: m/z (M+H)⁺=460.1.

### Step 2: (5S,8S,10aR)-benzyl 3-acetyl-5-amino-6-oxodecahydropyrrolo[1,2-a][1,5]diazocine-8-carboxylate

A mixture of (5S,8S,10aR)-benzyl 3-acetyl-5-amino-6-oxodecahydropyrrolo[1,2-a][1,5]diazocine-8-carboxylate (0.5 g, 1.1 mmol, 1.0 eq) in methylene chloride (2.5 mL) and HCl/dioxane (2.5 mL) was stirred at 25° C. for 2 hours. The reaction mixture was concentrated under reduced pressure to give (5S,8S,10aR)-benzyl 3-acetyl-5-amino-6-oxodecahydropyrrolo[1,2-a][1,5]diazocine-8-carboxylate (0.5 g, crude) as a white solid. LCMS: m/z (M+H)⁺=360.3.

### Step 3: (5S,8S,10aR)-benzyl 3-acetyl-5-(5-((diethoxyphosphoryl)difluoromethyl)benzo[b]thiophene-2-carboxamido)-6-oxodecahydropyrrolo[1,2-a][1,5]diazocine-8-carboxylate

To a solution of 5-((diethoxyphosphoryl)difluoromethyl)benzo[b]thiophene-2-carboxylic acid (0.34 g, 0.93 mmol, 1.0 eq) in dimethylformamide (1.0 mL) was added N,Ndiisopropylethylamine (0.48 g, 3.7 mmol, 4.0 eq) and HATU (0.53 g, 1.39 mmol, 1.5 eq). The mixture was stirred at 25° C. for 10 minutes. Then the solution of (5S,8S,10aR)-benzyl 3-acetyl-5-amino-6-oxodecahydropyrrolo[1,2-a][1,5]diazocine-8-carboxylate (0.4 g, 1.1 mmol, 1.2 eq) in dimethylformamide (1.0 mL) and N,Ndiisopropylethylamine (0.48 g, 3.7 mmol, 4.0 eq) was added. The reaction mixture was stirred 25° C. for 1 hour. The reaction was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The reaction mixture was concentrated under reduced pressure to give a residue. The residue was purified by flash silica gel chromatography to give (5S,8S,10aR)-benzyl 3-acetyl-5-(5-((diethoxyphosphoryl)difluoromethyl)benzo[b]thiophene-2-carboxamido)-6-oxodecahydropyrrolo[1,2-a][1,5]diazocine-8-carboxylate (0.3 g, 46% yield) as a yellow solid. LCMS: m/z (M+H)⁺=706.4.

### Step 4: (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylic Acid

A solution of (5S,8S,10aR)-benzyl 3-acetyl-5-(5-((diethoxyphosphoryl)difluoromethyl)benzo[b]thiophene-2-carboxamido)-6-oxodecahydropyrrolo[1,2-a][1,5]diazocine-8-carboxylate (0.4 g, 0.56 mmol, 1.0 eq) in methanol (20 mL) was degassed and purged with N₂ for 3 times. To the mixture was carefully added Pd/C (0.4 g), then the system was put under H₂ atmosphere and was stirred at 25° C. for 2 hours. The reaction mixture was filtered and the filter was concentrated to give a filtrate. The filtrate was concentrated under reduced pressure to give (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylic acid (0.3 g, crude) as an off-white oil, which was used in the next step without further purification.

### Representative Protocols:

Representative procedure for General Scheme 1: Synthesis of 1

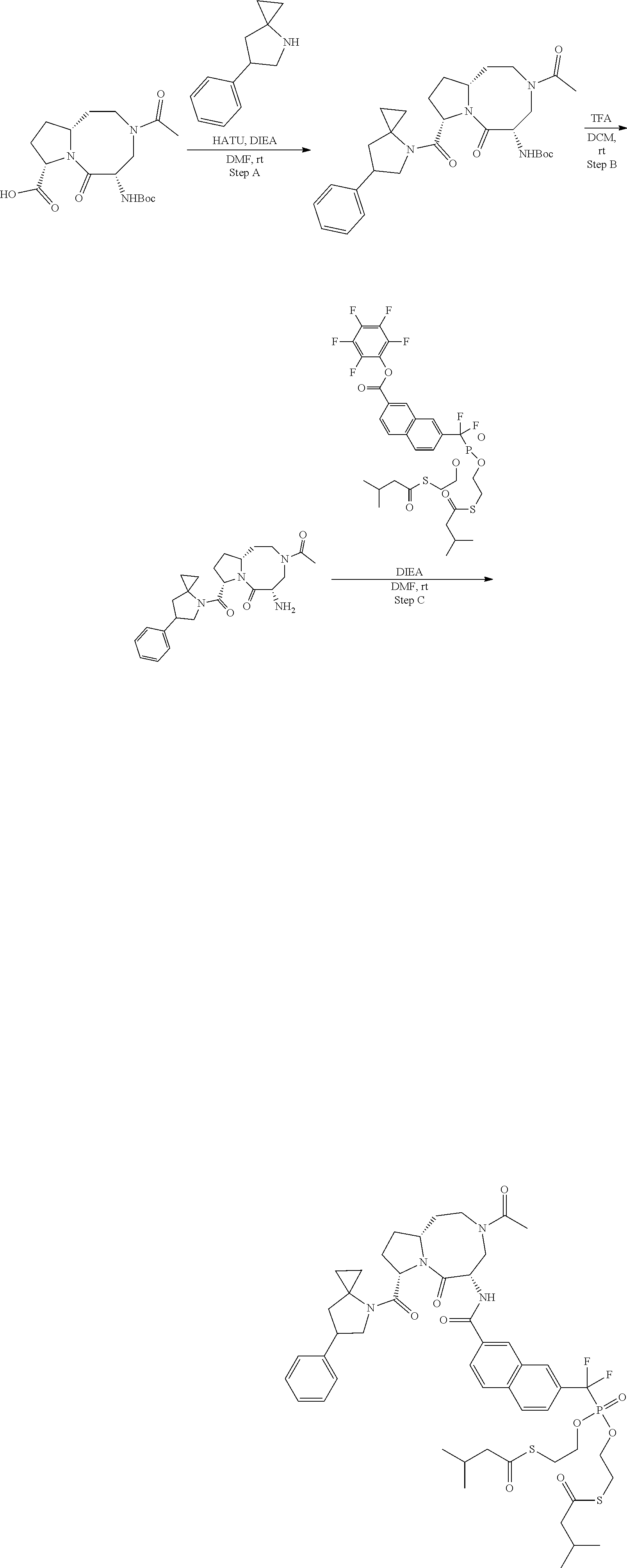

### Step A: tert-butyl ((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamate

A mixture of 6-phenyl-4-azaspiro[2.4]heptane (168 mg, 0.97 mmol, 1.2 eq.), (5S,8S,10aR)-3-acetyl-5-((tert-butoxycarbonyl)amino)-6-oxodecahydropyrrolo[1,2-a][1,5]diazocine-8-carboxylic acid (300 mg, 0.812 mmol, 1.0 eq.), HATU (463 mg, 1.218 mmol, 1.5 eq) and N,N-Diisopropylethylamine (246.8 mg, 2.43 mmol, 3.0 eq.) in DCM (10 mL) was stirred at room temperature for 3 hrs in a sealed tube. After completion, the reaction mixture quenched by adding H₂O (5 mL), extracted with EtOAc (5 mL×3) and the combined organic layers were washed with brine (5 mL×2), dried over with anhydrous Na₂SO₄, and concentrated under reduced pressure. The residue was purified by C18 column to afford tert-butyl ((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamate (80 mg, 152.5 μmol, 19%) as a white solid. LCMS (ESI): m/z=525.4 [M+H]⁺.

### Step B: (5S,8S,10aR)-3-acetyl-5-amino-8-(6-phenyl-4-azaspiro[2.4]heptane-4-carbonyl)octahydropyrrolo[1,2-a][1,5]diazocin-6(1H)-one

A solution of tert-butyl ((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamate (80 mg, 152.5 μmol, 1 eq.) in DCM (3 mL) and TFA (2 mL) was stirred at room temperature for 1 hr. After completion, the reaction was cooled with an ice-bath, then neutralized carefully with NaHCO₃(aq.) until the pH was adjusted to pH=8-9. The resulting mixture was extracted with DCM (10 mL×3), and the combined organic layers were washed with brine (10 mL×2), dried over with anhydrous Na₂SO₄, then concentrated under reduced pressure to give crude tert-butyl ((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamate (60 mg, 141.3 μmol) as a white solid, which was used in next step directly without further purification. LCMS (ESI): m/z=425.3 [M+H]⁺.

### Step C: S,S′-(((((7-(((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate)

To a solution of (5S,8S,10aR)-3-acetyl-5-amino-8-(6-phenyl-4-azaspiro[2.4]heptane-4-carbonyl)octahydropyrrolo[1,2-a][1,5]diazocin-6(1H)-one (30 mg, 70.7 μmol, 1 eq.) and DIEA (27.4 mg, 212 μmol, 3 eq.) in DMF (3 mL) was added perfluorophenyl 7-((bis(2-((3-methylbutanoyl)thio)ethoxy)phosphoryl)difluoromethyl)-2-naphthoate (53.5 mg, 70.7 μmol, 1 eq.). The resulting mixture was stirred at room temperature for 2 hrs, then the reaction was quenched by adding H₂O (10 mL) and extracted with DCM (10 mL×3). The combined organic layers were washed with brine (20 mL×2), dried over with anhydrous Na₂SO₄, then concentrated under reduced pressure. The residue was purified by C18 column to afford S,S′-(((((7-(((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate) (15.4 mg, 15.4 μmol, 22%) as a white solid. ¹H NMR (400 MHz, CDCl₃) δ 8.51-8.45 (m, 1H), 8.29-8.19 (m, 1H), 8.04-7.89 (m, 4H), 7.83-7.76 (m, 1H), 7.43-7.34 (m, 3H), 7.34-7.30 (m, 2H), 4.95-4.80 (m, 1H), 4.66-4.45 (m, 1H), 4.33-4.16 (m, 7H), 4.07-3.86 (m, 1H), 3.72-56 (m, 1H), 3.41-3.24 (m, 2H), 3.22-3.13 (m, 4H), 2.55-2.49 (m, 3H), 2.47-2.41 (m, 5H), 2.40-2.22 (m, 3H), 2.21-2.18 (m, 1H), 2.18-2.12 (m, 2H), 2.09-2.01 (m, 2H), 1.96-1.79 (m, 3H), 1.67 (s, 1H), 0.96 (d, J=6.6 Hz, 12H), 0.83-0.39 (m, 2H). LCMS (ESI): m/z=997.4 [M+H]⁺.

### Representative Procedure for General Scheme 2: Synthesis of 2

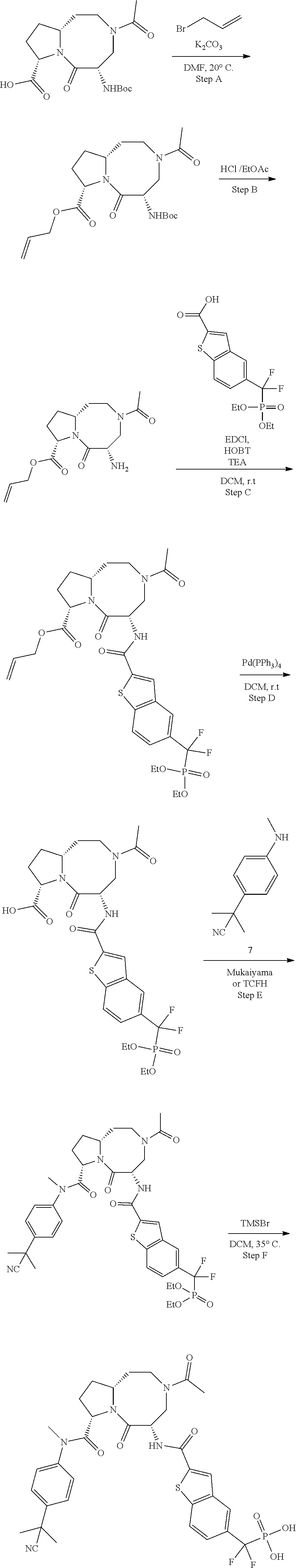

### Step A: allyl (5S,8S,10aR)-3-acetyl-5-((tert-butoxycarbonyl)amino)-6-oxodecahydropyrrolo[1,2-a][1,5]diazocine-8-carboxylate

To a solution of (5S,8S,10aR)-3-acetyl-5-((tert-butoxycarbonyl)amino)-6-oxodecahydropyrrolo[1,2-a][1,5]diazocine-8-carboxylic acid (3 g, 8.12 mmol, 1 eq.) and K₂CO₃ (2.25 g, 16.2 mmol, 2 eq.) in DMF (2 mL) was added 3-bromoprop-1-ene (1.17 g, 9.74 mmol, 1.2 eq.). The reaction mixture was stirred at 20° C. for 16 hrs. After completion, the reaction mixture was poured into water (10 mL), then extracted with EtOAc (20 mL×2). The organic layers were combined and washed with brine (150 mL), dried over anhydrous Na₂SO₄, and concentrated under reduced pressure. The residue was purified by C18 column to afford allyl (5S,8S,10aR)-3-acetyl-5-((tert-butoxycarbonyl)amino)-6-oxodecahydropyrrolo[1,2-a][1,5]diazocine-8-carboxylate (2.10 g, 5.12 mmol, 63%) as a white solid. LCMS (ESI): m/z=410.2 [M+H]⁺.

### Step B: allyl (5S,8S,10aR)-3-acetyl-5-amino-6-oxodecahydropyrrolo[1,2-a][1,5]diazocine-8-carboxylate

A solution of allyl (5S,8S,10aR)-3-acetyl-5-((tert-butoxycarbonyl)amino)-6-oxodecahydropyrrolo[1,2-a][1,5]diazocine-8-carboxylate (2.1 g, 5.12 mmol, 1 eq.) in HCl/EtOAc (15 mL) was stirred at room temperature for 3 hrs. After completion, the reaction mixture was concentrated under reduced pressure to afford allyl (5S,8S,10aR)-3-acetyl-5-amino-6-oxodecahydropyrrolo[1,2-a][1,5]diazocine-8-carboxylate (HCl salt) (1.30 g, 4.20 mmol, 82%) as a white solid, which was used in next step directly without further purification. LCMS (ESI): m/z=310.1 [M+H]⁺.

### Step C: allyl (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylate

To a solution of allyl (5S,8S,10aR)-3-acetyl-5-amino-6-oxodecahydropyrrolo[1,2-a][1,5]diazocine-8-carboxylate (1.3 g, 4.20 mmol, 1 eq.), 5-((diethoxyphosphoryl)difluoromethyl)benzo[b]thiophene-2-carboxylic acid (1.68 g, 4.62 mmol, 1.1 eq.) and TEA (1.27 g, 12.6 mmol, 3 eq.) in DCM (20 mL) was added EDCI (885 mg, 4.62 mmol, 1.1 eq.) and HOBt (574 mg, 4.62 mmol, 1.1 eq.) and the resulting mixture was stirred at room temperature overnight under nitrogen. After completion, the reaction was filtered and concentrated to get a residue, and the residue was purified by C18 column to get allyl (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylate (1.80 g, 2.74 mmol, 65%) as a white solid. LCMS (ESI): m/z=656.2 [M+H]⁺.

### Step D: (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylic Acid

A solution of allyl (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylate (1.8 g, 2.74 mmol, 1 eq.), Pd(PPh₃)₄ (158 mg, 137 μmol, 0.05 eq.) and pyrrolidine (194 mg, 2.74 mmol, 1 eq.) in DCM (20 mL) was stirred at room temperature overnight under nitrogen. The reaction was filtered and concentrated to provide a residue, which was purified by C18 column to get (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylic acid (1.28 g, 2.07 mmol, 76%) as a white solid. LCMS (ESI): m/z=616.1[M+H]⁺.

### Step E: diethyl ((2-(((5S,8S,10aR)-3-acetyl-8-((4-(2-cyanopropan-2-yl)phenyl)(methyl)carbamoyl)-6-oxodecahydropyrrolo[1,2-a][1,5]diazocin-5-yl)carbamoyl)benzo[b]thiophen-5-yl)difluoromethyl)phosphonate

A solution of (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylic acid (60 mg, 97.4 μmol, 1 eq.), 2-chloro-1-methylpyridin-1-ium iodide (29.6 mg, 116 μmol, 1.2 eq.), 2-methyl-2-(4-(methylamino)phenyl)propanenitrile (18.6 mg, 107 μmol, 1.1 eq.) and TEA (29.5 mg, 292 μmol, 3 eq.) in DCM (2 mL) was stirred at 35° C. for 30 hrs. After completion, the reaction was filtered and concentrated to get a residue, and the residue was purified by flash column chromatography on silica gel to afford diethyl ((2-(((5S,8S,10aR)-3-acetyl-8-((4-(2-cyanopropan-2-yl)phenyl)(methyl)carbamoyl)-6-oxodecahydropyrrolo[1,2-a][1,5]diazocin-5-yl)carbamoyl)benzo[b]thiophen-5-yl)difluoromethyl)phosphonate (25.0 mg, 32.3 μmol, 33%) as a white solid. LCMS (ESI): m/z=772 [M+H]⁺.

### Step F: ((2-(((5S,8S,10aR)-3-acetyl-8-((4-(2-cyanopropan-2-yl)phenyl)(methyl)carbamoyl)-6-oxodecahydropyrrolo[1,2-a][1,5]diazocin-5-yl)carbamoyl)benzo[b]thiophen-5-yl)difluoromethyl)phosphonic Acid

To a solution of diethyl ((2-(((5S,8S,10aR)-3-acetyl-8-((4-(2-cyanopropan-2-yl)phenyl)(methyl)carbamoyl)-6-oxodecahydropyrrolo[1,2-a][1,5]diazocin-5-yl)carbamoyl)benzo[b]thiophen-5-yl)difluoromethyl)phosphonate (23 mg, 29.7 μmol, 1 eq.) in DCM (2 mL) was added TMSBr (226 mg, 1.48 mmol, 50 eq.) and the resulting mixture was stirred at 35° C. for 30 hrs. After completion, the reaction was filtered and concentrated to get a residue, and the residue was purified by C18 column to afford ((2-(((5S,8S,10aR)-3-acetyl-8-((4-(2-cyanopropan-2-yl)phenyl)(methyl)carbamoyl)-6-oxodecahydropyrrolo[1,2-a][1,5]diazocin-5-yl)carbamoyl)benzo[b]thiophen-5-yl)difluoromethyl)phosphonic acid (20.0 mg, 27.9 μmol, 94%) as a white solid. ¹H NMR (400 MHz, CD₃OD) δ 8.06-7.85 (m, 3H), 7.70-7.62 (m, 1H), 7.53-7.33 (m, 4H), 5.20-5.10 (m, 1H), 4.42-3.66 (m, 6H), 3.25-3.15 (m, 3H), 2.34-2.21 (m, 3H), 2.12-1.76 (m, 6H), 1.65-1.49 (m, 6H). LCMS (ESI): m/z=716.1 [M+H]⁺.

### Representative Procedure for General Scheme 3: Synthesis of 3

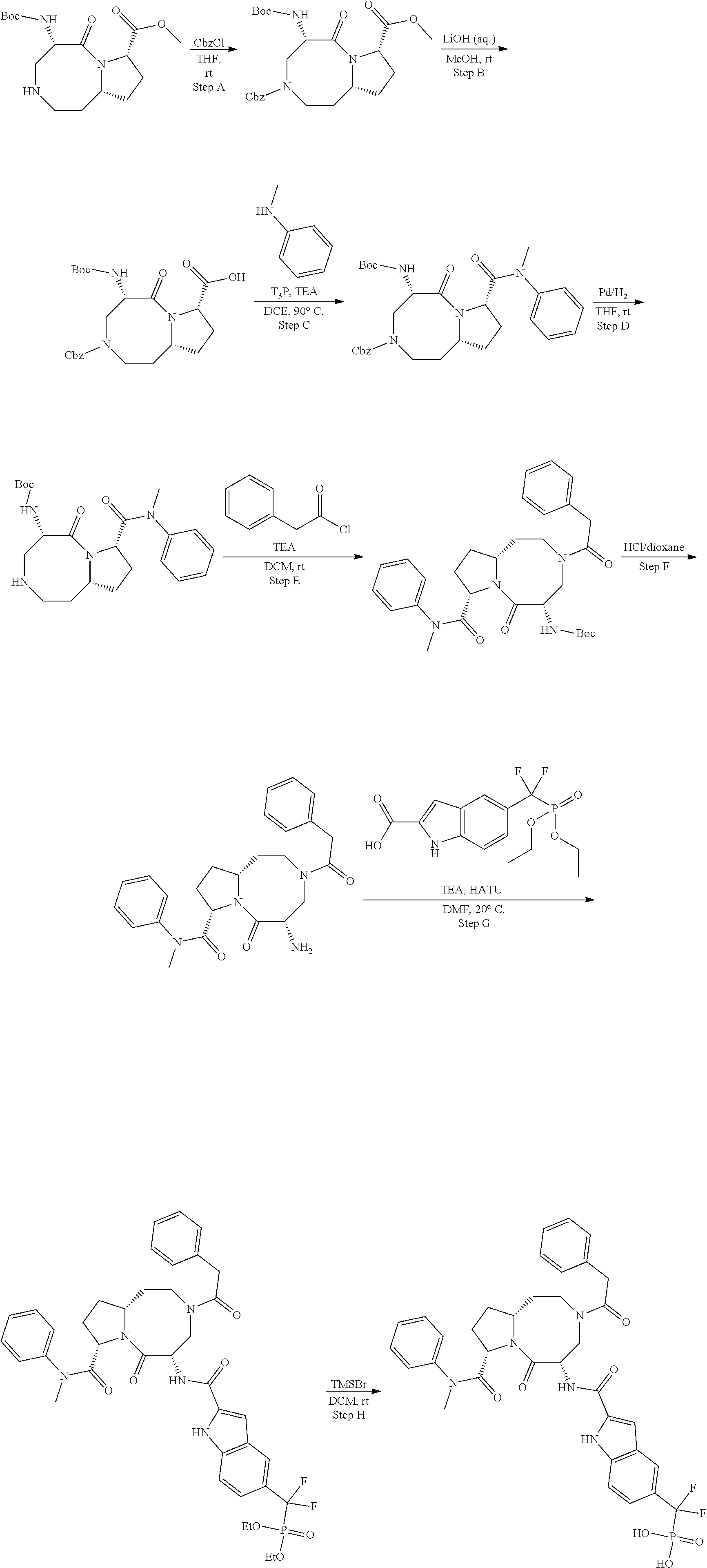

### Step A: 3-benzyl 8-methyl (5S,8S,10aR)-5-((tert-butoxycarbonyl)amino)-6-oxooctahydropyrrolo[1,2-a][1,5]diazocine-3,8(4H)-dicarboxylate

To a solution of methyl (5S,8S,10aR)-5-((tert-butoxycarbonyl)amino)-6-oxodecahydropyrrolo[1,2-a][1,5]diazocine-8-carboxylate (1 g, 2.92 mmol, 1 eq.) and NaHCO₃ (1.21 g, 14.5 mmol, 5 eq.) in a mixture of THF (10 mL) and water (2 mL) was added benzyl carbonochloridate (996 mg, 5.84 mmol, 2 eq.) at room temperature. After addition, the reaction mixture was stirred at room temperature for 3 hrs. After completion, the reaction mixture was poured into water, then extracted with EtOAc (100 mL×3). The organic layers were combined and washed with brine (50 mL), dried over anhydrous Na₂SO₄, and concentrated under reduced pressure to afford crude 3-benzyl 8-methyl (5S,8S,10aR)-5-((tert-butoxycarbonyl)amino)-6-oxooctahydropyrrolo[1,2-a][1,5]diazocine-3,8(4H)-dicarboxylate (1.10 g, 2.31 mmol, 80%) as a yellow oil. LCMS (ESI): m/z=476.2 [M+H]⁺.

### Step B: (5S,8S,10aR)-3-((benzyloxy)carbonyl)-5-((tert-butoxycarbonyl)amino)-6-oxodecahydropyrrolo[1,2-a][1,5]diazocine-8-carboxylic Acid

To a solution of 3-benzyl 8-methyl (5S,8S,10aR)-5-((tert-butoxycarbonyl)amino)-6-oxooctahydropyrrolo[1,2-a][1,5]diazocine-3,8(4H)-dicarboxylate (1.3 g, 2.73 mmol, 1 eq.) in a mixture of MeOH (5 mL) and water (1.5 mL) was added lithium hydroxide (544 mg, 13.6 mmol, 5 eq.) and the resulting mixture was stirred at room temperature for 12 hrs. After completion, the reaction mixture was concentrated under reduced pressure to give a residue. The residue was triturated from Et₂O (30 mL) to give (5S,8S,10aR)-3-((benzyloxy)carbonyl)-5-((tert-butoxycarbonyl)amino)-6-oxodecahydropyrrolo[1,2-a][1,5]diazocine-8-carboxylic acid (1.15 g, 2.49 mmol, 92%) as a white solid. LCMS (ESI): m/z=462.2 [M+H]⁺.

### Step C: benzyl (5S,8S,10aR)-5-((tert-butoxycarbonyl)amino)-8-(methyl(phenyl)carbamoyl)-6-oxooctahydropyrrolo[1,2-a][1,5]diazocine-3(4H)-carboxylate

To a solution of (5S,8S,10aR)-3-((benzyloxy)carbonyl)-5-((tert-butoxycarbonyl)amino)-6-oxodecahydropyrrolo[1,2-a][1,5]diazocine-8-carboxylic acid (400 mg, 866 μmol, 1 eq.), TEA (262 mg, 2.59 mmol, 3 eq.) and N-methylaniline (185 mg, 1.73 mmol, 2 eq.) in DCE (15 mL) was added T₃P (550 mg, 1.73 mmol, 2 eq.) at room temperature and the resulting mixture was heated at 90° C. for 20 hrs. After completion, the reaction mixture was poured into water (50 mL), then extracted with EtOAc (100 mL×3). The organic layers were combined and washed with brine (50 mL), dried over anhydrous Na₂SO₄, and concentrated under reduced pressure. The residue was purified by C18 column to afford benzyl (5S,8S,10aR)-5-((tert-butoxycarbonyl)amino)-8-(methyl(phenyl)carbamoyl)-6-oxooctahydropyrrolo[1,2-a][1,5]diazocine-3(4H)-carboxylate (400 mg, 726 μmol, 84%) as a white solid. LC-MS (ESI) m/z=551.2 [M+H]⁺.

### Step D: tert-butyl ((5S,8S,10aR)-8-(methyl(phenyl)carbamoyl)-6-oxodecahydropyrrolo[1,2-a][1,5]diazocin-5-yl)carbamate

To a solution of benzyl (5S,8S,10aR)-5-((tert-butoxycarbonyl)amino)-8-(methyl(phenyl)carbamoyl)-6-oxooctahydropyrrolo[1,2-a][1,5]diazocine-3(4H)-carboxylate (540 mg, 980 μmol, 1 eq.) in THF (20 mL) was added Pd/C (100 mg) under nitrogen. The suspension was degassed under vacuum and purged with H₂ several times. The resulting mixture was stirred at room temperature overnight. After completion, the suspension was filtered through a pad of Celite®, the filter cake was washed with THF (20 mL). The combined filtrates were concentrated to dryness to give tert-butyl ((5S,8S,10aR)-8-(methyl(phenyl)carbamoyl)-6-oxodecahydropyrrolo[1,2-a][1,5]diazocin-5-yl)carbamate (400 mg, 960 μmol, 98%) as a white solid. LCMS (ESI): m/z=417[M+H]⁺.

### Step E: tert-butyl ((5S,8S,10aR)-8-(methyl(phenyl)carbamoyl)-6-oxo-3-(2-phenylacetyl)decahydropyrrolo[1,2-a][1,5]diazocin-5-yl)carbamate

To a solution of tert-butyl ((5S,8S,10aR)-8-(methyl(phenyl)carbamoyl)-6-oxodecahydropyrrolo[1,2-a][1,5]diazocin-5-yl)carbamate (80 mg, 192 μmol, 1 eq.) and TEA (58.2 mg, 576 μmol, 3 eq.) in DCM (3 mL) was added 2-phenylacetyl chloride (44.5 mg, 288 μmol, 1.5 eq.) at 20° C. and the resulting mixture was stirred at 20° C. for 1 hr. After completion, the reaction mixture was poured into water (10 mL), then extracted with DCM (20 mL×3). The organic layers were combined and washed with brine (20 mL), dried over anhydrous Na₂SO₄, and concentrated under reduced pressure to afford crude tert-butyl ((5S,8S,10aR)-8-(methyl(phenyl)carbamoyl)-6-oxo-3-(2-phenylacetyl)decahydropyrrolo[1,2-a][1,5]diazocin-5-yl)carbamate (90.0 mg, 168 μmol, 88%) as a yellow solid, which was used in next step directly without further purification. LC-MS (ESI) m/z=573 [M+K]⁺.

### Step F: (5S,8S,10aR)-5-amino-N-methyl-6-oxo-N-phenyl-3-(2-phenylacetyl)decahydropyrrolo[1,2-a][1,5]diazocine-8-carboxamide

A solution of tert-butyl ((5S,8S,10aR)-8-(methyl(phenyl)carbamoyl)-6-oxo-3-(2-phenylacetyl)decahydropyrrolo[1,2-a][1,5]diazocin-5-yl)carbamate (90 mg, 168 μmol, 1 eq.) in HCl/dioxane (2 mL, 4 M) was stirred at 20° C. for 1 hr. After completion, the reaction mixture was concentrated under reduced pressure to give a residue. The residue was purified by C18 column to give (5S,8S,10aR)-5-amino-N-methyl-6-oxo-N-phenyl-3-(2-phenylacetyl)decahydropyrrolo[1,2-a][1,5]diazocine-8-carboxamide (70.0 mg, 161 μmol, 96%) as a white solid. LC-MS (ESI) m/z=435.2 [M+H]⁺.

### Step G: diethyl (difluoro(2-(((5S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonate

To a solution of (5S,8S,10aR)-5-amino-N-methyl-6-oxo-N-phenyl-3-(2-phenylacetyl)decahydropyrrolo[1,2-a][1,5]diazocine-8-carboxamide (69.8 mg, 161 μmol, 1 eq.), TEA (81.3 mg, 805 μmol, 5 eq.) and HATU (91.8 mg, 241 μmol, 1.5 eq.) in DMF (1 mL) was added 8 (61.4 mg, 177.1 μmol, 1.1 eq.), then the resulting mixture was stirred at 20° C. for 16 hrs. After completion, the reaction mixture was diluted with water (10 mL), then extracted with EtOAc (20 mL×3). The organic layers were combined and washed with brine (20 mL), dried over anhydrous Na₂SO₄, and concentrated under reduced pressure. The residue was purified by C18 column to afford diethyl (difluoro(2-(((5S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonate (120 mg, 157 μmol, 69%) as a white solid. LC-MS (ESI) m/z=764.1 [M+H]⁺.

### Step H: (difluoro(2-(((5S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonic Acid

To a solution of diethyl (difluoro(2-(((5S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonate (90 mg, 0.12 mmol, 1.0 eq.) in DCM (1 mL) was added TMSBr (179 mg, 1.17 mmol, 10 eq.) at room temperature. After addition, the reaction mixture was stirred at room temperature overnight. After completion, the reaction mixture was concentrated under reduced pressure. The residue was purified by RP-prep HPLC to give (difluoro(2-(((5S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonic acid (3) (50.0 mg, 70.6 μmol, 60%) as a yellow solid. LCMS (ESI): m/z=708.1 [M+H]⁺. ¹H NMR (400 MHz, DMSO-d₆) δ 12.37-11.72 (m, 1H), 8.89-8.29 (m, 1H), 7.98-7.82 (m, 1H), 7.57-7.13 (m, 13H), 5.09-4.91 (m, 1H), 4.32-4.20 (m, 2H), 4.08-4.00 (m, 1H), 3.96-3.89 (m, 2H), 3.81-3.75 (m, 1H), 3.42-3.31 (m, 2H), 3.20 (s, 3H), 2.20-1.53 (m, 6H).

### Representative Procedure for General Scheme 4: Synthesis of 4

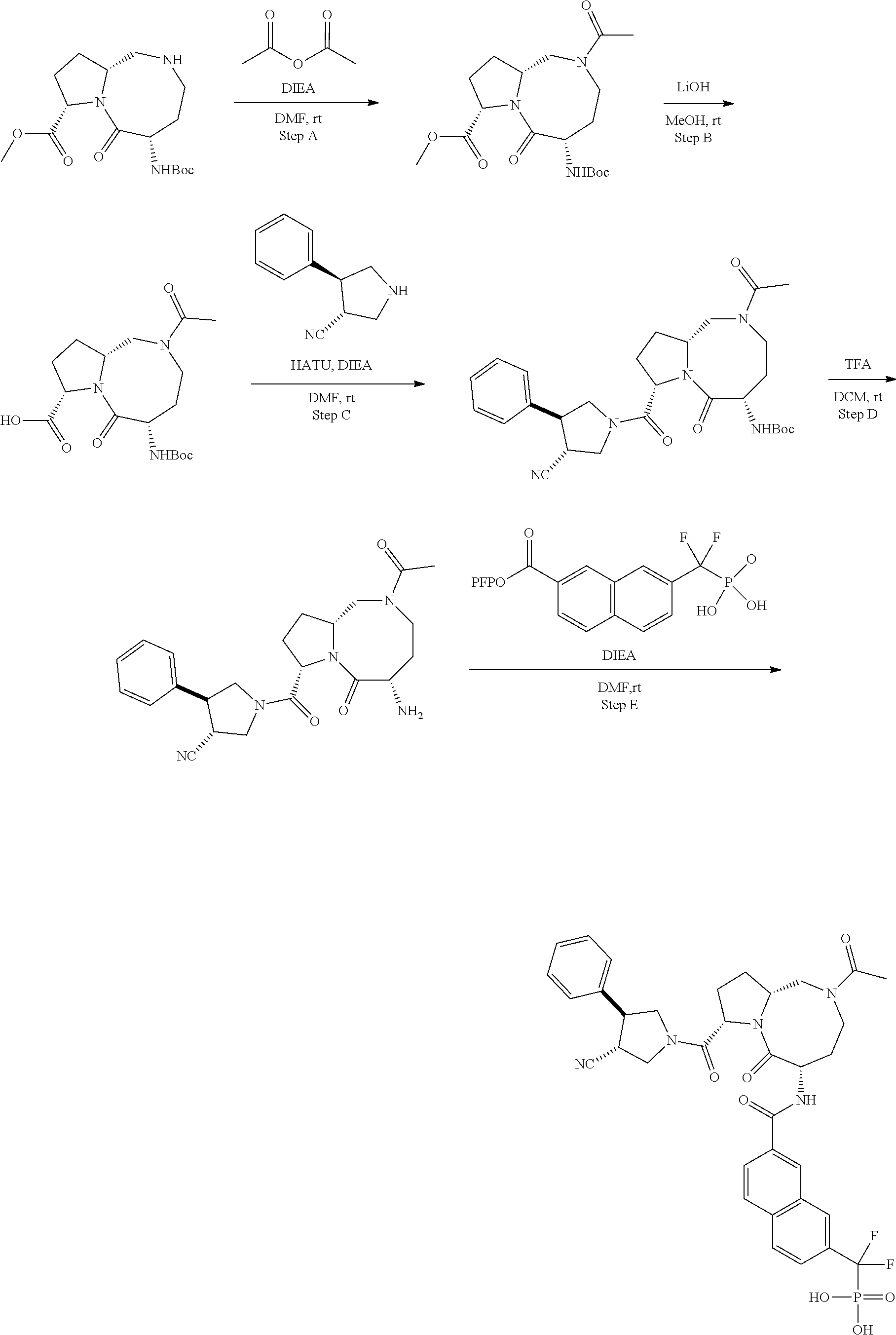

### Step A: methyl (5S,8S,10aR)-2-acetyl-5-((tert-butoxycarbonyl)amino)-6-oxodecahydropyrrolo[1,2-a][1,4]diazocine-8-carboxylate

To a solution of (5S,8S,10aR)-5-((tert-butoxycarbonyl)amino)-6-oxodecahydropyrrolo[1,2-a][1,4]diazocine-8-carboxylate (400 mg, 1.17 mmol, 1 eq.) and DIEA (453 mg, 3.51 mmol, 3 eq.) in DMF (6 mL) was added acetyl acetate (178 mg, 1.75 mmol, 1.5 eq.) and the resulting mixture was stirred at room temperature for 2 hrs. After completion, the reaction mixture was concentrated under reduced pressure. The residue was purified by flash column chromatography on silica gel to afford methyl (5S,8S,10aR)-2-acetyl-5-((tert-butoxycarbonyl)amino)-6-oxodecahydropyrrolo[1,2-a][1,4]diazocine-8-carboxylate (320 mg, 834 μmol, 71%) as a white solid. LCMS (ESI): m/z=384 [M+H]⁺.

### Step B: (5S,8S,10aR)-2-acetyl-5-((tert-butoxycarbonyl)amino)-6-oxodecahydropyrrolo[1,2-a][1,4]diazocine-8-carboxylic Acid

To a solution of (5S,8S,10aR)-2-acetyl-5-((tert-butoxycarbonyl)amino)-6-oxodecahydropyrrolo[1,2-a][1,4]diazocine-8-carboxylate (200 mg, 521 μmol, 1 eq.) in MeOH (8 mL) and water (2 mL) was added LiOH (37.3 mg, 1.56 mmol, 3 eq.) and the resulting mixture was stirred at room temperature for 12 hrs. After completion, the reaction mixture was concentrated under reduced pressure to afford (5S,8S,10aR)-2-acetyl-5-((tert-butoxycarbonyl)amino)-6-oxodecahydropyrrolo[1,2-a][1,4]diazocine-8-carboxylic acid (192 mg, 519 μmol, 100%) as a white solid. LCMS (ESI): m/z=370 [M+H]⁺.

### Step C: tert-butyl ((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamate

To a solution of (5S,8S,10aR)-2-acetyl-5-((tert-butoxycarbonyl)amino)-6-oxodecahydropyrrolo[1,2-a][1,4]diazocine-8-carboxylic acid (100 mg, 270 μmol, 1 eq.) and HATU (133 mg, 351 μmol, 1.3 eq.) in DMF (6 mL) were added DIEA (104 mg, 810 μmol, 3.0 eq.) and (rel-trans)-4-phenylpyrrolidine-3-carbonitrile (46.5 mg, 270 μmol, 1 eq.), then the resulting mixture was stirred at room temperature for 12 hrs. After completion, the reaction mixture was extracted with DCM (5 mL×3), and the combined organic layers were washed with brine (5 mL), dried over with anhydrous Na₂SO₄, then concentrated under reduced pressure, the residue was purified by C18 column to afford tert-butyl ((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamate (130 mg, 248 μmol, 92%) as a white solid. LCMS (ESI): m/z=524 [M+H]⁺.

### Step D: (rel-trans)-1-((5S,8S,10aR)-2-acetyl-5-amino-6-oxodecahydropyrrolo[1,2-a][1,4]diazocine-8-carbonyl)-4-phenylpyrrolidine-3-carbonitrile

To a solution of tert-butyl ((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamate (70 mg, 133 μmol, 1 eq.) in DCM (6 mL) was added TFA (3 mL) and the resulting mixture was stirred at room temperature for 1 hr. After completion, the reaction mixture was concentrated under reduced pressure to afford (rel-trans)-1-((5S,8S,10aR)-2-acetyl-5-amino-6-oxodecahydropyrrolo[1,2-a][1,4]diazocine-8-carbonyl)-4-phenylpyrrolidine-3-carbonitrile (TFA salt) (56.0 mg, 132 μmol, 99%) as a white solid, which was used in next step directly without further purification. LCMS (ESI): m/z=424 [M+H]⁺.

### Step E: ((7-(((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamoyl)naphthalen-2-yl)difluoromethyl)phosphonic Acid

To a solution of (rel-trans)-1-((5S,8S,10aR)-2-acetyl-5-amino-6-oxodecahydropyrrolo[1,2-a][1,4]diazocine-8-carbonyl)-4-phenylpyrrolidine-3-carbonitrile (TFA salt) (55.9 mg, 132 μmol, 1 eq.) and DIEA (51.1 mg, 396 μmol, 3.0 eq.) in DMF (6 mL) were added (difluoro(7-((perfluorophenoxy)carbonyl)naphthalen-2-yl)methyl)phosphonic acid (62 mg, 132 μmol, 1 eq.) and the resulting mixture was stirred at room temperature for 12 hrs. After completion, the reaction mixture was concentrated under reduced pressure. The residue was purified by C18 column to afford ((7-(((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamoyl)naphthalen-2-yl)difluoromethyl)phosphonic acid (14.9 mg, 21.0 μmol, 16%) as a white solid. ¹H NMR (400 MHz, DMSO-d₆) δ 8.89-8.72 (m, 1H), 8.69-8.56 (m, 1H), 8.19 (s, 1H), 8.12-7.98 (m, 3H), 7.72 (d, J=8.3 Hz, 1H), 7.51-7.24 (m, 5H), 4.82-4.65 (m, 2H), 4.53-3.69 (m, 11H), 2.36-1.75 (m, 9H). LCMS (ESI): m/z=708 [M+H]⁺.

### Alternative Chemistry Utilized in General Scheme 3 or 4: Core Amine Modification: Reductive Amination: General Procedure

### Preparation of Intermediate: Methyl (5S,8S,10aR)-5-((tert-butoxycarbonyl)amino)-3-(imidazo[1,2-a]pyridin-7-ylmethyl)-6-oxodecahydropyrrolo[1,2-a][1,5]diazocine-8-carboxylate

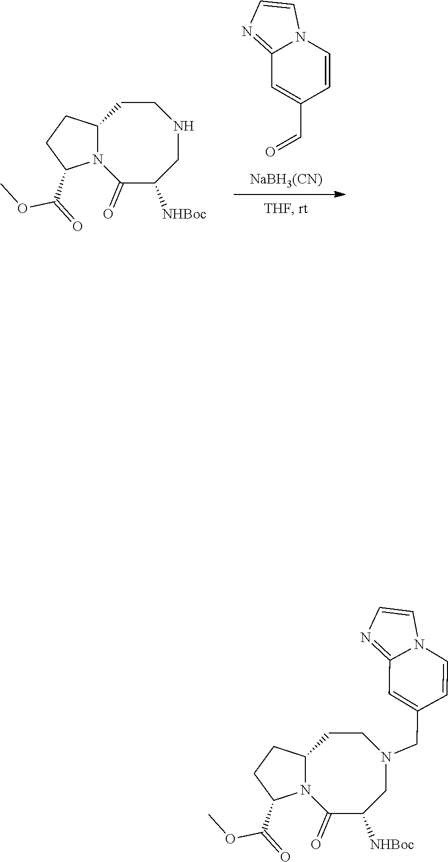 [SMILES: COC(=O)[C@@H]1CC[C@@H]2CCNC[C@H](NC(=O)OC(C)(C)C)C(=O)N21 ; "NaBH3(CN)" ; O=Cc1ccn2ccnc2c1 ; COC(=O)[C@@H]1CC[C@@H]2CCN(Cc3ccn4ccnc4c3)C[C@H](NC(=O)OC(C)(C)C)C(=O)N21]

A solution of methyl (5S,8S,10aR)-5-((tert-butoxycarbonyl)amino)-6-oxodecahydropyrrolo[1,2-a][1,5]diazocine-8-carboxylate (500 mg, 1.47 mmol, 1.0 eq.) and imidazo[1,2-a]pyridine-7-carbaldehyde (214 mg, 1.47 mmol, 1.0 eq.) in THF (15 mL) was stirred at room temperature for 30 min. NaBH₃(CN) (276.5 mg, 4.39 mmol, 3.0 eq.) was added in several portions, the resulting mixture was stirred for additional 12 hrs at room temperature. After completion, the reaction mixture was quenched with NH₄Cl (aq, 1N). The resulting mixture was extracted with DCM (10 mL×3), and the organic layers were combined and washed with brine (20 mL), dried over anhydrous Na₂SO₄, and concentrated under reduced pressure. The residue was purified by flash column chromatography on silica gel to afford methyl (5S,8S,10aR)-5-((tert-butoxycarbonyl)amino)-3-(imidazo[1,2-a]pyridin-7-ylmethyl)-6-oxodecahydropyrrolo[1,2-a][1,5]diazocine-8-carboxylate (212 mg, 0.45 mmol, 31%) as a white solid. LCMS (ESI) m/z=472.3 [M+H]⁺.

### Core Amine Modification: Alkylation: General Procedure

### Preparation of Intermediate: Methyl (5S,8S,10aR)-5-((tert-butoxycarbonyl)amino)-6-oxodecahydropyrrolo[1,2-a][1,5]diazocine-8-carboxylate

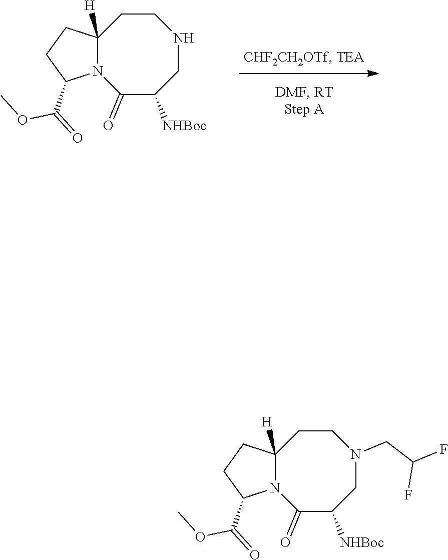 [SMILES: COC(=O)[C@@H]1CC[C@@H]2CCNC[C@H](NC(=O)OC(C)(C)C)C(=O)N21 ; COC(=O)[C@@H]1CC[C@@H]2CCN(CC(F)F)C[C@H](NC(=O)OC(C)(C)C)C(=O)N21]

A solution of methyl (5S,8S,10aR)-5-((tert-butoxycarbonyl)amino)-6-oxodecahydropyrrolo[1,2-a][1,5]diazocine-8-carboxylate (2 g, 5.85 mmol, 1 eq.), 2,2-difluoroethyl trifluoromethanesulfonate (2.50 g, 11.7 mmol, 2 eq.) and TEA (1.18 g, 11.7 mmol, 2 eq.) in DMF (20 mL) was stirred at room temperature for 12 hrs. After completion, the reaction mixture was concentrated under reduced pressure. The residue was purified by flash column chromatography on silica gel to afford methyl (5S,8S,10aR)-5-((tert-butoxycarbonyl)amino)-3-(2,2-difluoroethyl)-6-oxodecahydropyrrolo[1,2-a][1,5]diazocine-8-carboxylate (2.00 g, 4.93 mmol, 84%) as a white solid. LCMS (ESI): m/z=406 [M+H]⁺.

### Preparation of ((7-(((5S,8S,10aR)-3-acetyl-8-((3R,4R)-3-hydroxy-4-(1H-1,2,3-triazol-1-yl)pyrrolidine-1-carbonyl)-6-oxodecahydropyrrolo[1,2-a][1,5]diazocin-5-yl)carbamoyl)naphthalen-2-yl)difluoromethyl)phosphonic Acid (5)

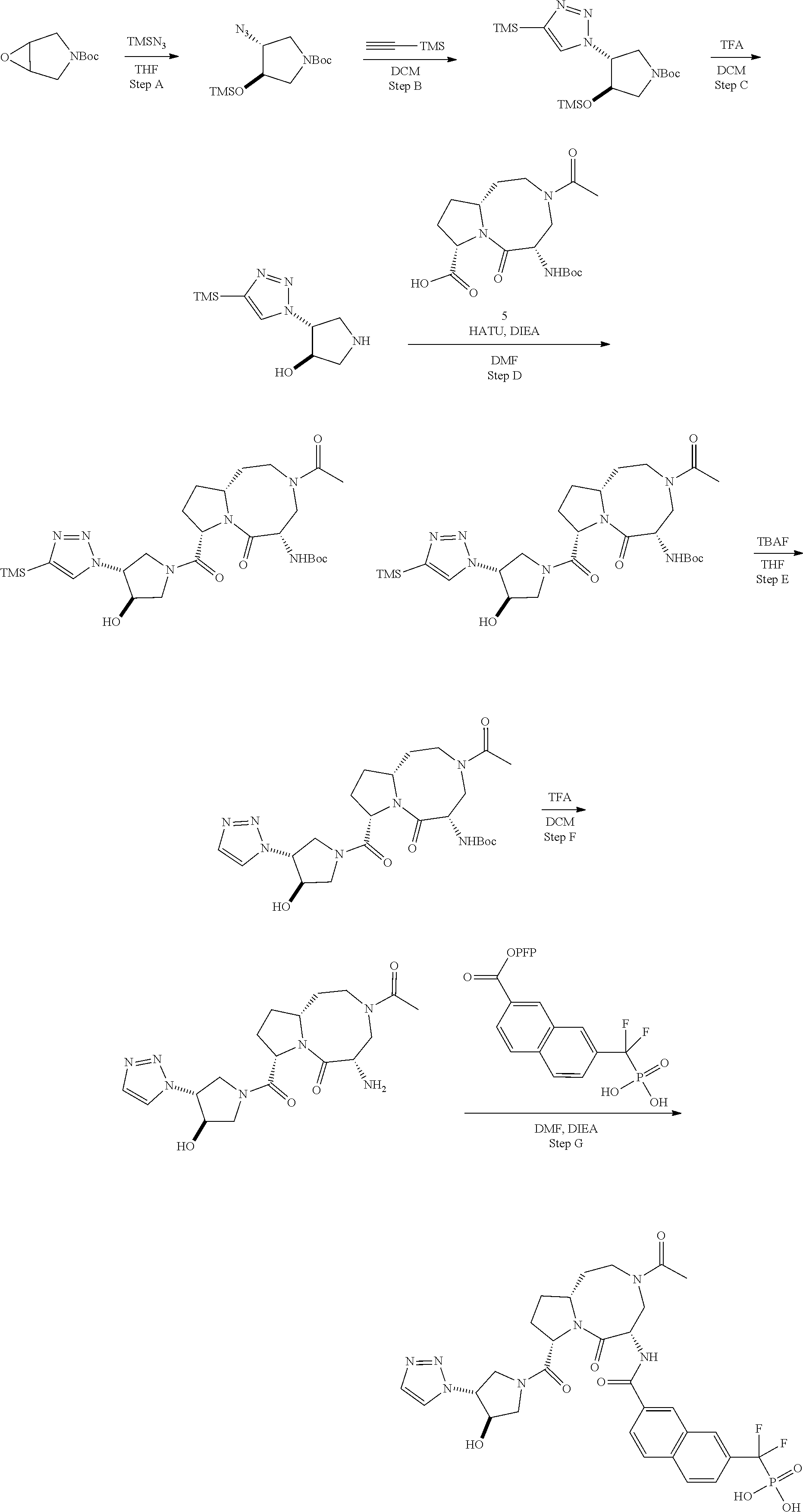

### Step A: tert-butyl (rel-trans)-3-azido-4-((trimethylsilyl)oxy)pyrrolidine-1-carboxylate

To a solution of tert-butyl 6-oxa-3-azabicyclo[3.1.0]hexane-3-carboxylate (3 g, 16.1 mmol, 1 eq.) and ammonium chloride (3.87 g, 72.4 mmol, 4.5 eq.) in MeOH (96 mL) and H₂O (12 mL) were added sodium TMSN₃ (5.23 g, 80.5 mmol, 5 eq.) at room temperature. The resulting mixture was stirred at 60° C. for an additional 18 hrs. After completion, the reaction mixture was diluted with water and extracted with EtOAc (2×100 mL). The combined organic layers were washed with NaHCO₃ solution, followed by water and then brine, dried over Na₂SO₄, filtered and concentrated to afford tert-butyl (rel-trans)-3-azido-4-((trimethylsilyl)oxy)pyrrolidine-1-carboxylate (3.60 g, 15.7 mmol, 98%) as colorless oil, which was used in next step directly without further purification. LCMS (ESI): m/z=173.2 [M+H−56]⁺.

### Step B: tert-butyl (rel-trans)-3-(4-(trimethylsilyl)-1H-1,2,3-triazol-1-yl)-4-((trimethylsilyl)oxy)pyrrolidine-1-carboxylate

To a solution of tert-butyl (rel-trans)-3-azido-4-((trimethylsilyl)oxy)pyrrolidine-1-carboxylate (3.6 g, 15.7 mmol, 1 eq.) and ethynyltrimethylsilane (3.85 g, 39.2 mmol, 2.5 eq.) in DCM (40 mL) was added [Ir(COD)Cl]₂ (105 mg, 157 μmol, 0.01 eq.) under N₂. The reaction mixture was stirred at room temperature for 24 hrs. After completion, the reaction was purified by flash column chromatography on silica gel to give tert-butyl (rel-trans)-3-(4-(trimethylsilyl)-1H-1,2,3-triazol-1-yl)-4-((trimethylsilyl)oxy)pyrrolidine-1-carboxylate (500 mg, 1.53 mmol, 10%) as brown oil. LCMS (ESI): m/z=327.2 [M+H]⁺.

### Step C: (rel-trans)-4-(4-(trimethylsilyl)-1H-1,2,3-triazol-1-yl)pyrrolidin-3-ol

To a solution of give tert-butyl (rel-trans)-3-(4-(trimethylsilyl)-1H-1,2,3-triazol-1-yl)-4-((trimethylsilyl)oxy)pyrrolidine-1-carboxylate (500 mg, 1.53 mmol, 1 eq.) in DCM (20 mL) was added TFA (1.74 g, 15.3 mmol, 10 eq.). The reaction mixture was stirred at room temperature for 1 hr. After completion, the reaction was cooled with an ice-bath, then neutralized carefully with NaHCO₃(aq.) until the pH was adjusted to pH=8-9. The resulting mixture was extracted with DCM (10 mL×3), and the combined organic layers were washed with brine (10 mL×2), dried over with anhydrous Na₂SO₄, then concentrated under reduced pressure to give crude (rel-trans)-4-(4-(trimethylsilyl)-1H-1,2,3-triazol-1-yl)pyrrolidin-3-ol (346 mg, 1.52 mmol, quant.) as a white solid, which was used in next step directly without further purification. LCMS (ESI): m/z=227.1 [M+H]⁺.

### Step D: tert-butyl ((5S,8S,10aR)-3-acetyl-8-((rel-trans)-3-hydroxy-4-(4-(trimethylsilyl)-1H-1,2,3-triazol-1-yl)pyrrolidine-1-carbonyl)-6-oxodecahydropyrrolo[1,2-a][1,5]diazocin-5-yl)carbamate

A solution of (5S,8S,10aR)-3-acetyl-5-((tert-butoxycarbonyl)amino)-6-oxodecahydropyrrolo[1,2-a][1,5]diazocine-8-carboxylic acid (296 mg, 802 μmol, 1.05 eq.) and HATU (319 mg, 840 μmol, 1.1 eq.) in DMF (5 mL) was stirred for 15 min, then DIEA (491 mg, 3.81 mmol, 5.0 eq.) and (rel-trans)-4-(4-(trimethylsilyl)-1H-1,2,3-triazol-1-yl)pyrrolidin-3-ol (173 mg, 764 μmol, 1 eq.) were added, and the resulting mixture was stirred at room temperature for additional 2 hrs. After completion, the reaction was purified by flash column chromatography on silica gel to afford tert-butyl ((5S,8S,10aR)-3-acetyl-8-((rel-trans)-3-hydroxy-4-(4-(trimethylsilyl)-1H-1,2,3-triazol-1-yl)pyrrolidine-1-carbonyl)-6-oxodecahydropyrrolo[1,2-a][1,5]diazocin-5-yl)carbamate (380 mg, 657 μmol, 86%) as a white solid. LCMS (ESI): m/z=578.2 [M+H]⁺.

### Step E: tert-butyl ((5S,8S,10aR)-3-acetyl-8-((rel-trans)-3-hydroxy-4-(1H-1,2,3-triazol-1-yl)pyrrolidine-1-carbonyl)-6-oxodecahydropyrrolo[1,2-a][1,5]diazocin-5-yl)carbamate

To a solution of tert-butyl ((5S,8S,10aR)-3-acetyl-8-((rel-trans)-3-hydroxy-4-(4-(trimethylsilyl)-1H-1,2,3-triazol-1-yl)pyrrolidine-1-carbonyl)-6-oxodecahydropyrrolo[1,2-a][1,5]diazocin-5-yl)carbamate (380 mg, 657 μmol, 1 eq.) in THF (4 mL) was added TBAF (515 mg, 1.97 mmol, 3 eq) dropwise at 0° C. The reaction mixture was stirred at room temperature overnight. After completion, the resulting mixture was poured into water. The aqueous layer was then extracted with EtOAc (10 mL×3), the combined organic layers were washed with brine (10 mL), dried over Na₂SO₄, filtered and concentrated under reduced pressure. The residue was purified by flash column chromatography on silica gel to afford tert-butyl ((5S,8S,10aR)-3-acetyl-8-((rel-trans)-3-hydroxy-4-(1H-1,2,3-triazol-1-yl)pyrrolidine-1-carbonyl)-6-oxodecahydropyrrolo[1,2-a][1,5]diazocin-5-yl)carbamate (300 mg, 593 μmol, 90%) as colorless solid. LCMS (ESI): m/z=506.2 [M+H]⁺.

### Step F: (5S,8S,10aR)-3-acetyl-5-amino-8-((rel-trans)-3-hydroxy-4-(1H-1,2,3-triazol-1-yl)pyrrolidine-1-carbonyl)octahydropyrrolo[1,2-a][1,5]diazocin-6(1H)-one

To a solution of tert-butyl ((5S,8S,10aR)-3-acetyl-8-((rel-trans)-3-hydroxy-4-(1H-1,2,3-triazol-1-yl)pyrrolidine-1-carbonyl)-6-oxodecahydropyrrolo[1,2-a][1,5]diazocin-5-yl)carbamate (300 mg, 593 μmol, 1.0 eq.) in DCM (3 mL) was added TFA (676 mg, 5.93 mmol, 10 eq.), and the resulting mixture was stirred at room temperature for 2 hrs. After completion, the reaction mixture was cooled down in an ice-bath, then neutralized carefully with NaHCO₃(aq.) until the pH was adjusted to pH=8-9. The resulting mixture was extracted with DCM (10 mL×3), and the combined organic layers were washed with brine (10 mL×2), dried over with anhydrous Na₂SO₄, then concentrated under reduced pressure to give crude (5S,8S,10aR)-3-acetyl-5-amino-8-((rel-trans)-3-hydroxy-4-(1H-1,2,3-triazol-1-yl)pyrrolidine-1-carbonyl)octahydropyrrolo[1,2-a][1,5]diazocin-6(1H)-one (180 mg, 443 μmol, 75%) as a white solid, which was used in next step directly without further purification. LCMS (ESI): m/z=406.2 [M+H]⁺.

### Step G: ((7-(((5S,8S,10aR)-3-acetyl-8-((rel-trans)-3-hydroxy-4-(1H-1,2,3-triazol-1-yl)pyrrolidine-1-carbonyl)-6-oxodecahydropyrrolo[1,2-a][1,5]diazocin-5-yl)carbamoyl)naphthalen-2-yl)difluoromethyl)phosphonic Acid

To a solution of (difluoro(7-((perfluorophenoxy)carbonyl)naphthalen-2-yl)methyl)phosphonic acid (50 mg, 123 μmol, 1.0 eq.) and (5S,8S,10aR)-3-acetyl-5-amino-8-((rel-trans)-3-hydroxy-4-(1H-1,2,3-triazol-1-yl)pyrrolidine-1-carbonyl)octahydropyrrolo[1,2-a][1,5]diazocin-6(1H)-one (57.1 mg, 122 μmol, 1.0 eq.) in DMF (1.5 mL) was added DIEA (47.6 mg, 369 μmol, 3.0 eq.) and the mixture was stirred at room temperature for an additional 18 hrs. After completion, the reaction mixture was concentrated under reduced pressure. The residue was purified by RP-prep-HPLC to give ((7-(((5S,8S,10aR)-3-acetyl-8-((rel-trans)-3-hydroxy-4-(1H-1,2,3-triazol-1-yl)pyrrolidine-1-carbonyl)-6-oxodecahydropyrrolo[1,2-a][1,5]diazocin-5-yl)carbamoyl)naphthalen-2-yl)difluoromethyl)phosphonic acid (11.0 mg, 15.9 μmol, 13%) as a white solid. ¹H NMR (400 MHz, DMSO-d₆) δ 8.92-8.48 (m, 2H), 8.29-7.79 (m, 5H), 7.76-7.64 (m, 1H), 5.16-5.00 (m, 1H), 4.67-4.52 (m, 1H), 4.37-4.26 (m, 1H), 4.21-4.02 (m, 1H), 4.02-3.81 (m, 2H), 3.80-3.46 (m, 4H), 3.43-3.22 (m, 2H), 3.16-3.00 (m, 1H), 2.31-2.15 (m, 2H), 2.13-2.08 (m, 1H), 2.02-1.90 (m, 1H), 1.86-1.69 (m, 2H), 1.25-1.21 (m, 3H). LCMS (ESI): m/z=690.3 [M+H]⁺.

### Preparation of ((2-(((5S,8S,10aR)-3-acetyl-8-(((3S,4S)-4-hydroxy-1-(methylsulfonyl)pyrrolidin-3-yl)carbamoyl)-6-oxodecahydropyrrolo[1,2-a][1,5]diazocin-5-yl)carbamoyl)benzo[b]thiophen-5-yl)difluoromethyl)phosphonic Acid (6)

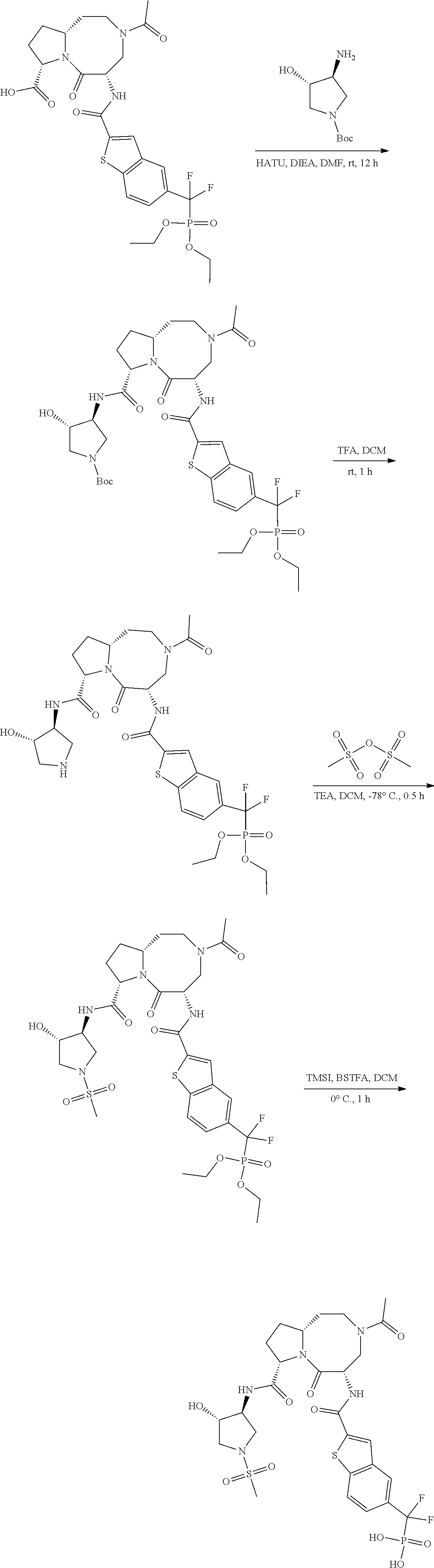

### Step 1: (3S,4S)-ter-butyl 3-((5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxamido)-4-hydroxypyrrolidine-1-carboxylate

To a solution of (5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxylic acid (0.2 g, 0.32 mmol, 1 eq) in DMF (1 mL) was added HATU (0.16 g, 0.42 mmol, 1.3 eq) and DIEA (0.17 g, 1.3 mmol, 4 eq). The mixture was stirred at 20° C. for 1 hour. The reaction solution turned brown. To the above mixture was added tert-butyl (3S,4S)-3-amino-4-hydroxypyrrolidine-1-carboxylate (78 mg, 0.39 mmol, 1.2 eq). The mixture was stirred at 20° C. for 12 hours. The reaction mixture was filtered and the filtrate was purified by prep-HPLC (column: Phenomenex Gemini 150×25 mm×10 um, mobile phase: water (0.2% FA)-ACN; B %: 34%-64%, 10 min) followed by lyophilization to give tert-butyl (3S,4S)-3-((5S,8S,10aR)-3-acetyl-5-(5-((diethoxyphosphoryl)difluoromethyl)benzo[b]thiophene-2-carboxamido)-6-oxodecahydropyrrolo[1,2-a][1,5]diazocine-8-carboxamido)-4-hydroxypyrrolidine-1-carboxylate (0.15 g, 57% yield) as a red solid. LCMS: m/z (M+H)⁺=800.5. ¹H NMR (400 MHz, CD₃OD) δ 8.20-7.98 (m, 3H), 7.76-7.59 (m, 1H), 5.49 (s, 2H), 5.13 (m, 1H), 4.40 (m, 2H), 4.28-4.08 (m, 6H), 4.02-3.90 (m, 1H), 3.84-3.50 (m, 5H), 2.28 (s, 3H), 2.23-1.77 (m, 6H), 1.52-1.41 (m, 9H), 1.32 (t, J=7.2 Hz, 6H).

### Step 2: diethyl ((2-(((5S,8S,10aR)-3-acetyl-8-(((3S,4S)-4-hydroxypyrrolidin-3-yl)carbamoyl)-6-oxodecahydropyrrolo[1,2-a][1,5]diazocin-5-yl)carbamoyl)benzo[b]thiophen-5-yl)difluoromethyl) phosphonate

To a solution of (3S,4S)-tert-butyl 3-((5S,8S,10aR)-3-acetyl-5-(5-((diethoxyphosphoryl)difluoromethyl) benzo[b]thiophene-2-carboxamido)-6-oxodecahydropyrrolo[1,2-a][1,5]diazocine-8-carboxamido)-4-hydroxypyrrolidine-1-carboxylate (0.15 g, 187 μmol, 1 eq) in methylene chloride (1 mL) was added trifluoroacetic acid (0.2 mL). The mixture was stirred at 20° C. for 1 hour. The reaction mixture was concentrated under reduced pressure to give diethyl ((2-(((5S,8S,10aR)-3-acetyl-8-(((3S,4S)-4-hydroxypyrrolidin-3-yl)carbamoyl)-6-oxodecahydropyrrolo[1,2-a][1,5]diazocin-5-yl)carbamoyl) benzo[b]thiophen-5-yl)difluoromethyl) phosphonate (0.1 mg, crude) as a yellow oil and was used into the next step without further purification

### Step 3: diethyl ((2-(((5S,8S,10aR)-3-acetyl-8-(((3S,4S)-4-hydroxy-1-(methylsulfonyl)pyrrolidin-3-yl)carbamoyl)-6-oxodecahydropyrrolo[1,2-a][1,5]diazocin-5-yl)carbamoyl)benzo[b]thiophen-5-yl)difluoromethyl)phosphonate

To a solution of diethyl ((2-(((5S,8S,10aR)-3-acetyl-8-(((3S,4S)-4-hydroxypyrrolidin-3-yl)carbamoyl)-6-oxodecahydropyrrolo[1,2-a][1,5]diazocin-5-yl)carbamoyl)benzo[b]thiophen-5-yl)difluoromethyl) phosphonate (0.1 g, 0.14 mmol, 1 eq) in methylene chloride (0.5 mL) and triethylamine (14 mg, 0.14 mmol, 1.0 eq) was added methanesulfonic anhydride (24.7 mg, 142 μmol, 1.0 eq) at 0° C. The mixture was stirred at 0° C. for 30 minutes to give a transparent solution. The reaction mixture was filtered, and the filtrate was concentrated to give a residue. The residue was purified by flash silica gel chromatography to give diethyl ((2-(((5S,8S,10aR)-3-acetyl-8-(((3S,4S)-4-hydroxy-1-(methylsulfonyl)pyrrolidin-3-yl) carbamoyl)-6-oxodecahydropyrrolo[1,2-a][1,5]diazocin-5-yl)carbamoyl)benzo[b]thiophen-5-yl) difluoromethyl)phosphonate (0.1 g, 90% yield) as a white solid. LCMS: m/z (M+H)⁺=778.4

### Step 4: ((2-(((5S,8S,10aR)-3-acetyl-8-(((3S,4S)-4-hydroxy-1-(methylsulfonyl)pyrrolidin-3-yl)carbamoyl)-6-oxodecahydropyrrolo[1,2-a][1,5]diazocin-5-yl)carbamoyl)benzo[b]thiophen-5-yl)difluoromethyl)phosphonic Acid

To a solution of diethyl ((2-(((5S,8S,10aR)-3-acetyl-8-(((3S,4S)-4-hydroxy-1-(methylsulfonyl)pyrrolidin-3-yl)carbamoyl)-6-oxodecahydropyrrolo[1,2-a][1,5]diazocin-5-yl)carbamoyl)benzo[b]thiophen-5-yl)difluoromethyl)phosphonate (0.1 g, 0.13 mmol, 1 eq) in methylene chloride (1 mL) was added bstfa (0.19 g, 0.77 mmol, 6 eq) and trimethylsilyl iodide (0.10 g, 0.51 mmol, 4 eq) at 0° C. The mixture was stirred at 0° C. for 1 hour to give a bronzing solution. The reaction mixture was filtered and the filtrate was concentrated to give a residue. The residue was purified by prep-HPLC (column: Phenomenex Luna C18 150×25 mm×10 um, mobile phase: water (0.1% TFA)-ACN; B %:5%-35%, 10 min) to lyophilization to give ((2-(((5S,8S,10aR)-3-acetyl-8-(((3S,4S)-4-hydroxy-1-(methylsulfonyl) pyrrolidin-3-yl)carbamoyl)-6-oxodecahydropyrrolo[1,2-a][1,5]diazocin-5-yl)carbamoyl) benzo[b]thiophen-5-yl)difluoromethyl)phosphonic acid (9.9 mg, 10% yield) as a white solid. LCMS: m/z (M+H)⁺=722.4. 1H NMR (400 MHz, CD₃OD-d₄) δ 8.17 (s, 1H), 8.15-8.11 (m, 1H), 8.05-7.96 (m, 1H), 7.68 (d, J=8.4 Hz, 1H), 5.14 (dd, J=4.0, 10.8 Hz, 1H), 4.41 (m, 2H), 4.23-4.05 (m, 2H), 4.01-3.73 (m, 2H), 3.72-3.52 (m, 4H), 3.34 (d, J=2.0 Hz, 1H), 3.28 (s, 1H), 2.97-2.86 (m, 3H), 2.35-2.21 (m, 4H), 2.19-2.08 (m, 1H), 2.07-1.88 (m, 3H), 1.87-1.77 (m, 1H).

### Synthesis of ((2-(((5R,8S,10aR)-8-((3S,4R)-3-cyano-4-phenylpyrrolidine-1-carbonyl)-3-imino-3-oxido-6-oxodecahydro-3l4-pyrrolo[2,1-d][1,5]thiazocin-5-yl)carbamoyl)benzo[b]thiophen-5-yl)difluoromethyl)phosphonic Acid (7)

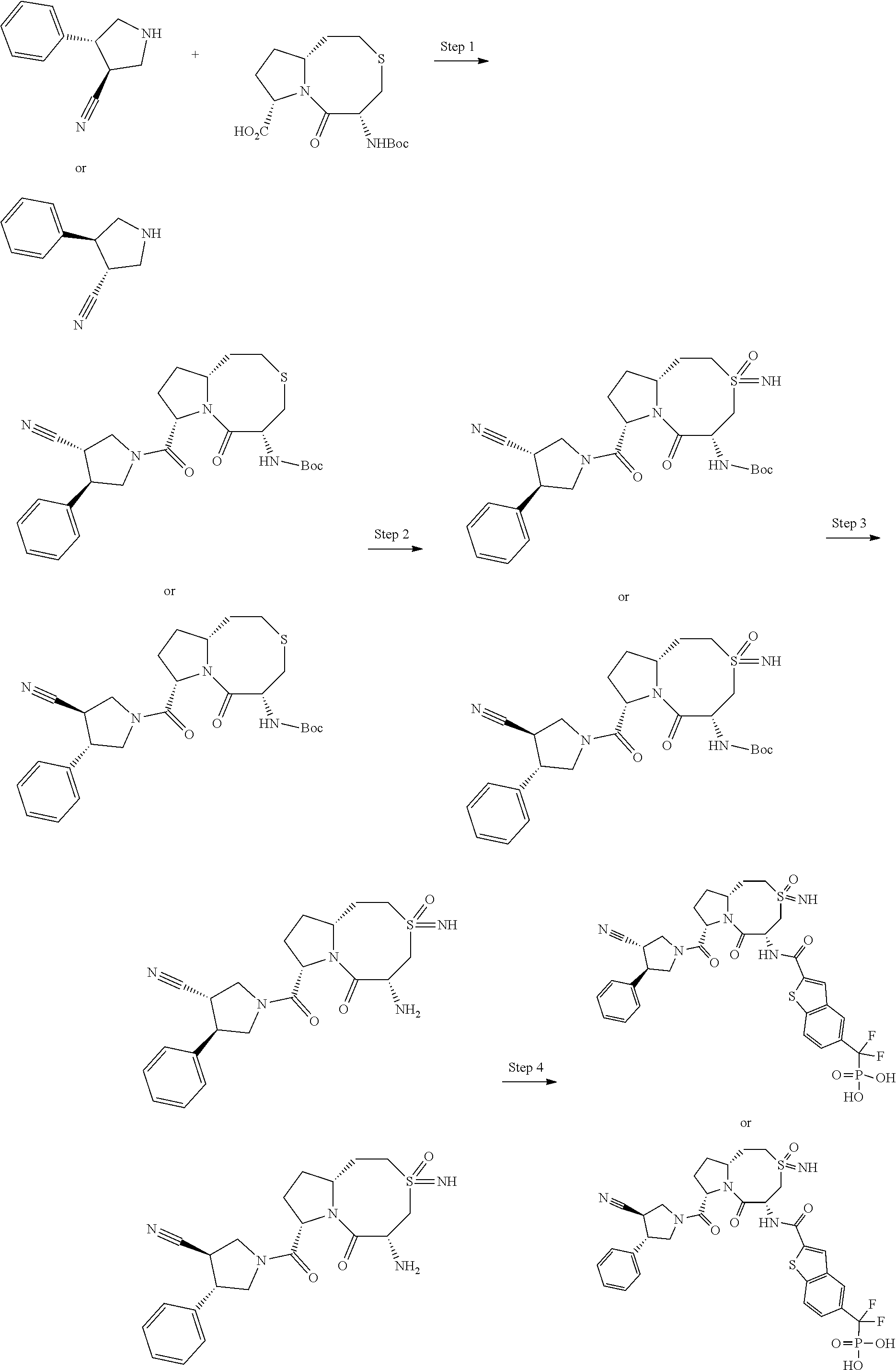

### Step 1: Preparation of tert-butyl ((5R,8S,10aR)-8-((3S,4R or 3R,4S)-3-cyano-4-phenylpyrrolidine-1-carbonyl)-6-oxooctahydro-4H-pyrrolo[2,1-d][1,5]thiazocin-5-yl)carbamate

To a mixture of (3S,4R or 3R,4S)-4-phenylpyrrolidine-3-carbonitrile hydrochloride (0.05 g, 239 μmol, 1 eq) (pyrrolidine SFC peak 2 used), Et₃N (72.5 mg, 0.717 mmol, 3_eq) and tert-butyl (5R,8S,10aR)-5-((tert-butoxycarbonyl)amino)-6-oxooctahydro-4H-pyrrolo[2,1-d][1,5]thiazocine-8-carboxylate (82.3 mg, 0.239 mmol, 1 eq) in DMF (2 mL) was treated with tripropyl-1,3,5,2λ⁵,4λ⁵,6λ⁵-trioxatriphosphinane-2,4,6-trione (304 mg, 0.478 mmol, 2 eq). The resulting mixture was stirred at ambient temperature for 12 h and purified by HPLC, to afford tert-butyl ((5R,8S,10aR)-8-((3S,4R or 3R,4S)-3-cyano-4-phenylpyrrolidine-1-carbonyl)-6-oxooctahydro-4H-pyrrolo[2,1-d][1,5]thiazocin-5-yl)carbamate (47.5 mg, 0.0952 mmol, 39.9% yield).

### Step 2: Preparation of tert-butyl ((5R,8S,10aR)-8-((3S,4R or 3R,4S)-3-cyano-4-phenylpyrrolidine-1-carbonyl)-3-imino-3-oxido-6-oxodecahydro-3l4-pyrrolo[2,1-d][1,5]thiazocin-5-yl)carbamate

A mixture of tert-butyl ((5R,8S,10aR)-8-((3S,4R or 3R,4S)-3-cyano-4-phenylpyrrolidine-1-carbonyl)-6-oxooctahydro-4H-pyrrolo[2,1-d][1,5]thiazocin-5-yl)carbamate (0.05 g, 0.100 mmol, 1 eq) and ammonium acetate (12.3 mg, 0.160 mmol, 1.6 eq) in MeOH (1 mL) was stirred at ambient temperature for 5 minutes, and then added (acetyloxy)(phenyl)-λ³-iodanyl acetate (80.5 mg, 0.250 mmol, 2.5 eq). The resulting mixture was stirred at ambient temperature for 12 h and purified by HPLC to afford tert-butyl ((5R,8S,10aR)-8-((3S,4R or 3R,4S)-3-cyano-4-phenylpyrrolidine-1-carbonyl)-3-imino-3-oxido-6-oxodecahydro-3l4-pyrrolo[2,1-d][1,5]thiazocin-5-yl)carbamate (34.2 mg, 0.0645 mmol, 64.6% yield).

### Step 3: Preparation of (3S,4R or 3R,4S)-1-((5R,8S,10aR)-5-amino-3-imino-3-oxido-6-oxodecahydro-3l4-pyrrolo[2,1-d][1,5]thiazocine-8-carbonyl)-4-phenylpyrrolidine-3-carbonitrile

To a solution of tert-butyl ((5R,8S,10aR)-8-((3S,4R or 3R,4S)-3-cyano-4-phenylpyrrolidine-1-carbonyl)-3-imino-3-oxido-6-oxodecahydro-3l4-pyrrolo[2,1-d][1,5]thiazocin-5-yl)carbamate (0.030 g, 0.0566 mmol, 1 eq) in DCM (5 mL) was added TFA (25.7 mg, 0.226 mmol, 4 eq. After stirring for 10 h, the reaction mixture was concentrated in vacuo and dried on an oil pump to afford (3S,4R or 3R,4S)-1-((5R,8S,10aR)-5-amino-3-imino-3-oxido-6-oxodecahydro-3l4-pyrrolo[2,1-d][1,5]thiazocine-8-carbonyl)-4-phenylpyrrolidine-3-carbonitrile (27.0 mg, 0.0496 mmol, 87.9% yield, TFA salt).

### Step 4: Preparation of ((2-(((5R,8S,10aR)-8-((3S,4R)-3-cyano-4-phenylpyrrolidine-1-carbonyl)-3-imino-3-oxido-6-oxodecahydro-3l4-pyrrolo[2,1-d][1,5]thiazocin-5-yl)carbamoyl)benzo[b]thiophen-5-yl)difluoromethyl)phosphonic Acid

To a solution of (3S,4R or 3R,4S)-1-((5R,8S,10aR)-5-amino-3-imino-3-oxido-6-oxodecahydro-3l4-pyrrolo[2,1-d][1,5]thiazocine-8-carbonyl)-4-phenylpyrrolidine-3-carbonitrile (0.03 g, 0.0551 mmol, 1 eq, TFA salt) and triethylamine (27.8 mg, 0.275 mmol, 5 eq) in DMF (0.5 mL) was added 4-nitrophenyl 5-((bis((trimethylsilyl)oxy)phosphoryl)difluoromethyl)benzo[b]thiophene-2-carboxylate (34.7 mg, 0.0606 mmol, 1.1 eq). After stirring for 24 h, the reaction mixture was purified by HPLC to yield ((2-(((5R,8S,10aR)-8-((3S,4R or 3R,4S)-3-cyano-4-phenylpyrrolidine-1-carbonyl)-3-imino-3-oxido-6-oxodecahydro-3l4-pyrrolo[2,1-d][1,5]thiazocin-5-yl)carbamoyl)benzo[b]thiophen-5-yl)difluoromethyl)phosphonic acid (9.00 mg, 0.0125 mmol, 22.7% yield). LCMS (ESI): m/z=718.0 [M−H]⁻.

### Synthesis of ((2-(((5R,8S,10aR)-8-((3S,4R)-3-cyano-4-phenylpyrrolidine-1-carbonyl)-3,3-dioxido-6-oxooctahydro-4H-pyrrolo[2,1-d][1,5]thiazocin-5-yl)carbamoyl)benzo[b]thiophen-5-yl)difluoromethyl)phosphonic Acid (8)

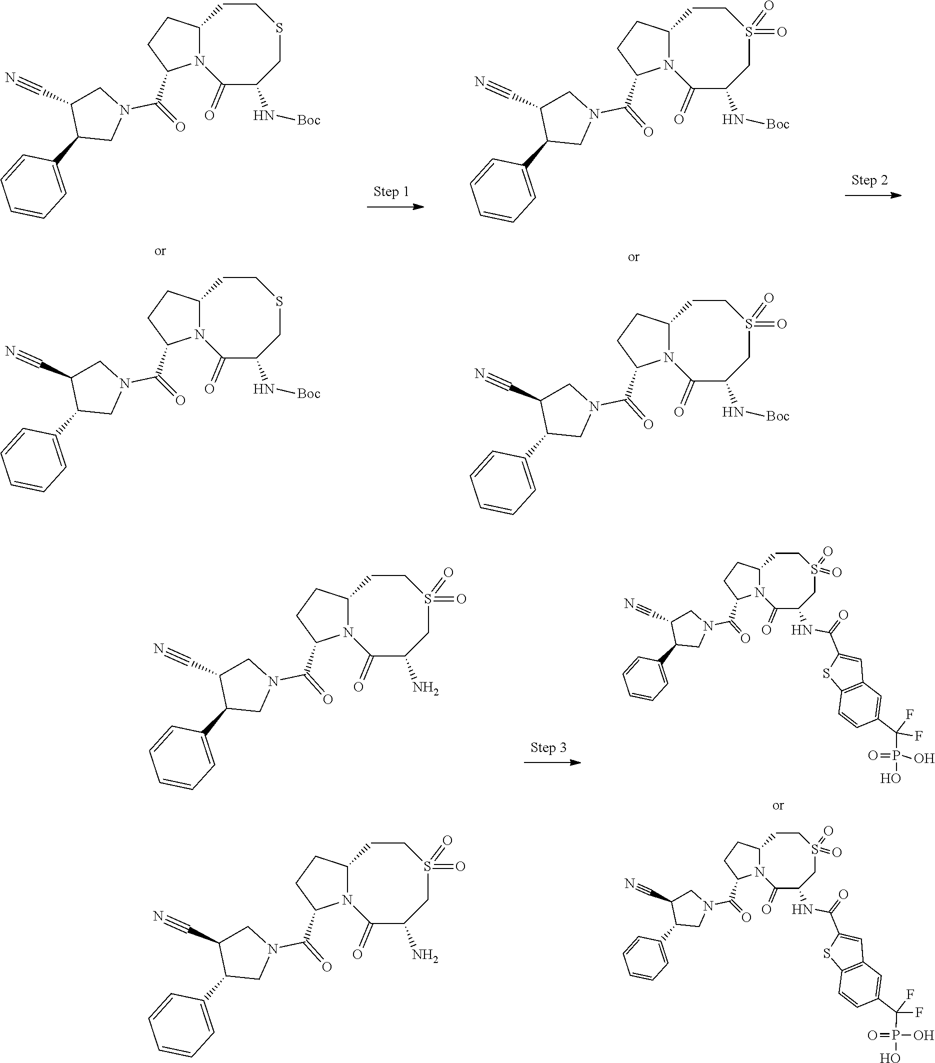

### Step 1: Preparation of tert-butyl ((5R,8S,10aR)-8-((3S,4R or 3R,4S)-3-cyano-4-phenylpyrrolidine-1-carbonyl)-3,3-dioxido-6-oxooctahydro-4H-pyrrolo[2,1-d][1,5]thiazocin-5-yl)carbamate

To a cooled (0° C.) solution of tert-butyl ((5R,8S,10aR)-8-((3S,4R or 3R,4S)-3-cyano-4-phenylpyrrolidine-1-carbonyl)-6-oxooctahydro-4H-pyrrolo[2,1-d][1,5]thiazocin-5-yl)carbamate (0.07 g, 0.140 mmol, 1 eq) in DCM (3 mL) was added mCPBA (85.1 mg, 0.0420 mmol, 3 eq) in one portion. The reaction mixture was stirred and allowed to warm to room temperature. After stirring for 24 h, the product was purified by HPLC to yield tert-butyl ((5R,8S,10aR)-8-((3S,4R or 3R,4S)-3-cyano-4-phenylpyrrolidine-1-carbonyl)-3,3-dioxido-6-oxooctahydro-4H-pyrrolo[2,1-d][1,5]thiazocin-5-yl)carbamate (0.249 g, 0.555 mmol, 39.6% yield).

### Step 2: Preparation of (3S,4R or 3R,4S)-1-((5R,8S,10aR)-5-amino-3,3-dioxido-6-oxooctahydro-4H-pyrrolo[2,1-d][1,5]thiazocine-8-carbonyl)-4-phenylpyrrolidine-3-carbonitrile

To a solution of tert-butyl ((5R,8S,10aR)-8-((3S,4R or 3R,4S)-3-cyano-4-phenylpyrrolidine-1-carbonyl)-3,3-dioxido-6-oxooctahydro-4H-pyrrolo[2,1-d][1,5]thiazocin-5-yl)carbamate (0.045, 0.0848 mmol, 1 eq) in DCM (2 mL) was added TFA (0.0965 mg, 0.847 mmol, 4 eq). After stirring for 12 h, the reaction mixture was concentrated in vacuo and dried on an oil pump to afford (3S,4R or 3R,4S)-1-((5R,8S,10aR)-5-amino-3,3-dioxido-6-oxooctahydro-4H-pyrrolo[2,1-d][1,5]thiazocine-8-carbonyl)-4-phenylpyrrolidine-3-carbonitrile (TFA salt). The product was subjected to the next reaction without further purification.

### Step 4: Preparation of ((2-(((5R,8S,10aR)-8-((3S,4R or 3R,4S)-3-cyano-4-phenylpyrrolidine-1-carbonyl)-3,3-dioxido-6-oxooctahydro-4H-pyrrolo[2,1-d][1,5]thiazocin-5-yl)carbamoyl)benzo[b]thiophen-5-yl)difluoromethyl)phosphonic Acid

To a solution of (3S,4R or 3R,4S)-1-((5R,8S,10aR)-5-amino-3,3-dioxido-6-oxooctahydro-4H-pyrrolo[2,1-d][1,5]thiazocine-8-carbonyl)-4-phenylpyrrolidine-3-carbonitrile (TFA salt) and triethylamine (128 mg, 1.27 mmol, 15 eq) in DMF (0.5 mL) was added 4-nitrophenyl 5-((bis((trimethylsilyl)oxy)phosphoryl)difluoromethyl)benzo[b]thiophene-2-carboxylate (48.6 mg, 0.0848 mmol, 1 eq). After stirring for 24 h, the reaction mixture was purified by HPLC to yield ((2-(((5R,8S,10aR)-8-((3S,4R)-3-cyano-4-phenylpyrrolidine-1-carbonyl)-3,3-dioxido-6-oxooctahydro-4H-pyrrolo[2,1-d][1,5]thiazocin-5-yl)carbamoyl)benzo[b]thiophen-5-yl)difluoromethyl)phosphonic acid (28.5 mg, 39.5 μmol, 46.6% yield). LCMS (ESI): m/z=719.2 [M−H]⁻.

### Synthesis of ((2-(((5S,8S,10aR)-3-((benzyloxy)carbonyl)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic acid (9) and ((2-(((5S,8S,10aR)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic Acid (10)

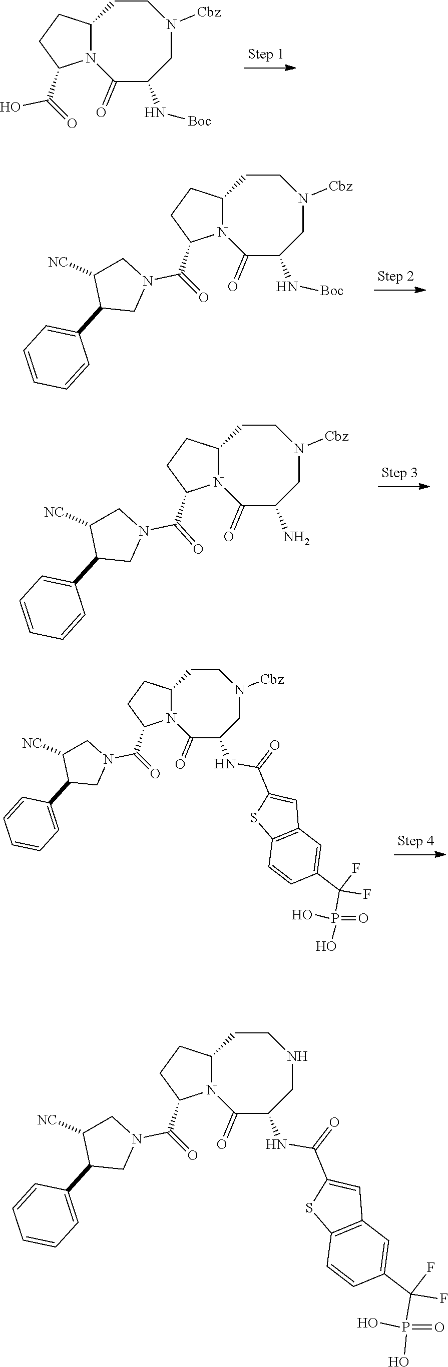 [SMILES: CC(C)(C)OC(=O)N[C@H]1CN(C(=O)OCc2ccccc2)CC[C@H]2CC[C@@H](C(=O)O)N2C1=O ; CC(C)(C)OC(=O)N[C@H]1CN(C(=O)OCc2ccccc2)CC[C@H]2CC[C@@H](C(=O)N3C[C@@H](C#N)[C@H](c4ccccc4)C3)N2C1=O ; N#C[C@@H]1CN(C(=O)[C@@H]2CC[C@@H]3CCN(C(=O)OCc4ccccc4)C[C@H](N)C(=O)N32)C[C@H]1c1ccccc1 ; N#C[C@@H]1CN(C(=O)[C@@H]2CC[C@@H]3CCN(C(=O)OCc4ccccc4)C[C@H](NC(=O)c4cc5cc(C(F)(F)P(=O)(O)O)ccc5s4)C(=O)N32)C[C@H]1c1ccccc1 ; N#C[C@@H]1CN(C(=O)[C@@H]2CC[C@@H]3CCNC[C@H](NC(=O)c4cc5cc(C(F)(F)P(=O)(O)O)ccc5s4)C(=O)N32)C[C@H]1c1ccccc1]

### Step 1: Preparation of benzyl (5S,8S,10aR)-5-((tert-butoxycarbonyl)amino)-8-((rel-trans)-3-cyano-4-phenylpyrrolidine-1-carbonyl)-6-oxooctahydropyrrolo[1,2-a][1,5]diazocine-3(4H)-carboxylate

To a mixture of (rel-trans)-4-phenylpyrrolidine-3-carbonitrile hydrochloride (67.8 mg, 0.325 mmol, 1 eq), triethylamine (1.29 mmol, 4 eq) and (5S,8S,10aR)-3-[(benzyloxy)carbonyl]-5-{[(tert-butoxy)carbonyl]amino}-6-oxo-decahydropyrrolo[1,2-a][1,5]diazocine-8-carboxylic acid (0.15 g, 0.325 mmol, 1 eq) in dichloromethane (5 mL) was treated with tripropyl-1,3,5,2λ⁵,4λ⁵,6λ⁵-trioxatriphosphinane-2,4,6-trione (412 mg, 0.650 mmol, 2 eq). After stirring for 18 h, the mixture partitioned with H2O. The organic layer was washed twice with H₂O (2×5 mL), dried over Na₂SO₄, filtered, and concentrated to afford benzyl (5S,8S,10aR)-5-((tert-butoxycarbonyl)amino)-8-((rel-trans)-3-cyano-4-phenylpyrrolidine-1-carbonyl)-6-oxooctahydropyrrolo[1,2-a][1,5]diazocine-3(4H)-carboxylate (168 mg, 0.272 mmol, 84.0% yield).

### Step 2: Preparation of benzyl (5S,8S,10aR)-5-amino-8-((rel-trans)-3-cyano-4-phenylpyrrolidine-1-carbonyl)-6-oxooctahydropyrrolo[1,2-a][1,5]diazocine-3(4H)-carboxylate

To benzyl (5S,8S,10aR)-5-((tert-butoxycarbonyl)amino)-8-((rel-trans)-3-cyano-4-phenylpyrrolidine-1-carbonyl)-6-oxooctahydropyrrolo[1,2-a][1,5]diazocine-3(4H)-carboxylate (77.7 mg, 0.682 mmol, 2 eq) in DCM (5 mL) was added TFA. After stirring for 10 h, the reaction mixture was concentrated in vacuo to yield benzyl (5S,8S,10aR)-5-amino-8-((rel-trans)-3-cyano-4-phenylpyrrolidine-1-carbonyl)-6-oxooctahydropyrrolo[1,2-a][1,5]diazocine-3(4H)-carboxylate (160 mg, 0.254 mmol, 74.7% yield, TFA salt).

### Step 3: Preparation of ((2-(((5S,8S,10aR)-3-((benzyloxy)carbonyl)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic Acid (9)

To a solution of benzyl (5S,8S,10aR)-5-amino-8-((rel-trans)-3-cyano-4-phenylpyrrolidine-1-carbonyl)-6-oxooctahydropyrrolo[1,2-a][1,5]diazocine-3(4H)-carboxylate (0.2 g, 0.317 mmol, 1 eq) and triethylamine (127 mg, 1.26 mmol, 4 eq) in DMF (0.5 mL) was added 4-nitrophenyl 5-((bis((trimethylsilyl)oxy)phosphoryl)difluoromethyl)benzo[b]thiophene-2-carboxylate (181 mg, 0.317 mmol, 1 eq). After stirring for 24 h, the mixture was purified on HPLC to yield ((2-(((5S,8S,10aR)-3-((benzyloxy)carbonyl)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic acid (50.9 mg, 0.0632 mmol, 19.9% yield). LCMS (ESI): m/z=806 [M+H]⁺; ¹H NMR (400 MHz, DMSO-d₆) δ 9.05-8.90 (m, 1H), 8.38-8.25 (m, 1H), 8.18-8.05 (m, 2H), 7.60 (d, J=8.4 Hz, 1H), 7.48-7.23 (m, 10H), 5.21-4.85 (m, 3H), 4.68-4.51 (m, 1H), 4.41-4.15 (m, 2H), 4.12-3.74 (m, 4H), 3.72-3.53 (m, 4H), 3.34-3.11 (m, 1H), 2.40-2.22 (m, 1H), 2.10-1.70 (m, 5H).

### Step 3: Preparation of ((2-(((5S,8S,10aR)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic Acid (10)

To a suspension of ((2-(((5S,8S,10aR)-3-((benzyloxy)carbonyl)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic acid (0.03 g, 0.0372 mmol, 1 eq) and 10% Pd\C (30 mg) in 1:1 mixture of MeOH/THF (3.3 mL) was subjected to three cycles of evacuation and purging with H₂ (g). The reaction suspension was subsequently stirred under H₂ (g) (1 atm). After stirring for 6 h, the mixture was purged under N₂ (g) and filtered over a pad of Celite®. The filtrate was concentrated to give ((2-(((5S,8S,10aR)-8-((rel-trans)-3-cyano-4-phenylpyrrolidine-1-carbonyl)-6-oxodecahydropyrrolo[1,2-a][1,5]diazocin-5-yl)carbamoyl)benzo[b]thiophen-5-yl)difluoromethyl)phosphonic acid (3.60 mg, 0.005 mmol, 14.4% yield). ¹H NMR (600 MHz, DMSO-d₆) δ 8.22 (s, 1H), 8.12 (s, 1H), 8.03 (s, 1H), 7.94 (s, 1H), 7.62 (d, J=8.1 Hz, 1H), 7.41 (m, 4H), 7.32 (s, 2H), 4.34 (m, 2H), 4.23-4.06 (m, 2H), 3.97 (m, 2H), 3.81 (s, 2H), 3.69 (d, J=9.9 Hz, 2H), 2.06 (s, 3H), 1.84 (s, 4H), 1.04 (t, J=7.0 Hz, 2H).

### Phosphonate Acids Data Tables

### Prepared According to General Scheme 1: Phosphonic Acids

The following compounds in Table 14 were prepared according to the representative procedure described above for the synthesis of S,S′-(((((7-(((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate) (1) and utilizing the appropriate starting materials and modifications.

TABLE 14

| Com- | | | | |
|---|---|---|---|---|
| pound | Name | Structure | LCMS | NMR |
| 11 | ((2- (((5S,8S,10aR)- 3-acetyl- 6-oxo-8-(6- phenyl-2,6- diazabicyclo [3.2.0]heptane- 2- carbonyl)deca- hydropyrrolo [1,2- a][1,5]diazocin- 5- yl)carbamoyl) benzo[b]thio- phen-5- yl)difluorometh- yl)phosphon- ic acid | 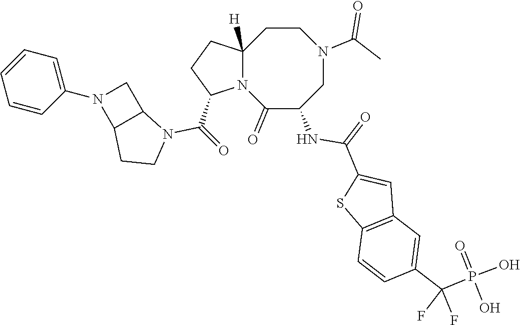 [SMILES: CC(=O)N1CC[C@H]2CC[C@@H](C(=O)N3CCC4C3CN4c3ccccc3)N2C(=O)[C@@H](NC(=O)c2cc3cc(C(F)(F)P(=O)(O)O)ccc3s2)C1] | 716.3 [M + H]⁺ | ¹H NMR (400 MHz, METHANOL-d₄) δ 8.19- 8.09 (m, 2H), 8.06- 7.97 (m, 1H), 7.70-7.62 (m, 1H), 7.22-7.05 (m, 2H), 6.76-6.64 (m, 1H), 6.56-6.42 (m, 2H), 5.17- 4.97 (m, 1H), 4.82- 4.72 (m, 1H), 4.71-4.55 (m, 2H), 4.50-3.93 (m, 4H), 3.92-3.47 (m, 5H), 2.55-2.37 (m, 1H), 2.36- 2.15 (m, 5H), 2.15- 1.82 (m, 5H). |
| 12 | ((2- (((5S,8S,10aR)- 3-acetyl- 8-((3S,4′S or 3R,4′R)-4′- cyano-2- oxospiro[indo- line-3,3′- pyrrolidine]- 1′-carbonyl)- 6- oxodecahydro- pyrrolo[1,2- a][1,5]diazocin- 5- yl)carbamoyl) benzo[b]thio- phen-5- yl)difluorometh- yl)phosphon- | 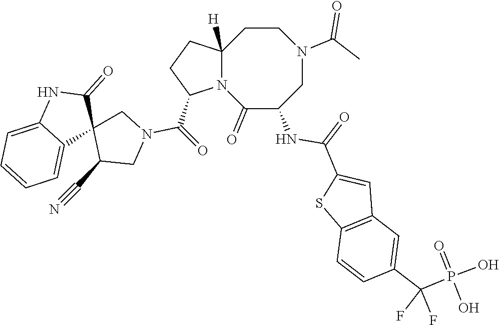 [SMILES: CC(=O)N1CC[C@H]2CC[C@@H](C(=O)N3C[C@@H](C#N)[C@]4(C3)C(=O)Nc3ccccc34)N2C(=O)[C@@H](NC(=O)c2cc3cc(C(F)(F)P(=O)(O)O)ccc3s2)C1] | 755.2 [M + H]⁺ | ¹H NMR (400 MHz, METHANOL-d₄) δ 8.20- 8.09 (m, 2H), 8.07- 7.97 (m, 1H), 7.73-7.64 (m, 1H), 7.52-7.26 (m, 2H), 7.19-6.92 (m, 2H), 5.19-5.02 (m, 1H), 4.83- 4.65 (m, 1H), 4.54- 4.08 (m, 3H), 4.05-3.52 (m, 7H), 2.60-2.34 (m, 1H), 2.33-2.19 (m, 4H), 2.18-1.83 (m, 4H). |
| | ic acid | or | | |
| | | 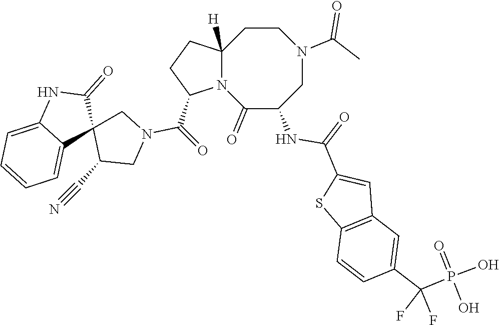 [SMILES: CC(=O)N1CC[C@H]2CC[C@@H](C(=O)N3C[C@H](C#N)[C@@]4(C3)C(=O)Nc3ccccc34)N2C(=O)[C@@H](NC(=O)c2cc3cc(C(F)(F)P(=O)(O)O)ccc3s2)C1] | | |
| | | SFC peak 2 was used for | | |
| | | biological testing | | |
| 13 | ((2- (((5S,8S,10aR)- 3-acetyl- 8-((3R,4′R or 3S,4′S)-4′- cyano-2- oxospiro[indo- line-3,3′- pyrrolidine]- 1′-carbonyl)- 6- oxodecahydro- pyrrolo[1,2- a][1,5]diazocin- 5- yl)carbamoyl) benzo[b]thio- phen-5- yl)difluorometh- yl)phosphon- | 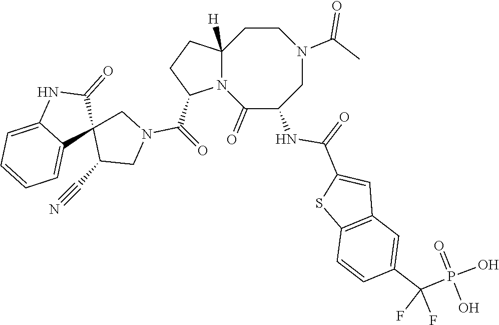 [SMILES: CC(=O)N1CC[C@H]2CC[C@@H](C(=O)N3C[C@H](C#N)[C@@]4(C3)C(=O)Nc3ccccc34)N2C(=O)[C@@H](NC(=O)c2cc3cc(C(F)(F)P(=O)(O)O)ccc3s2)C1] | 755.2 [M + H]⁺ | ¹H NMR (400 MHz, METHANOL-d₄) δ 8.24- 8.12 (m, 2H), 8.08- 8.00 (m, 1H), 7.76-7.56 (m, 2H), 7.39-7.23 (m, 1H), 7.22-7.09 (m, 1H), 7.05-6.96 (m, 1H), 5.21- 5.06 (m, 1H), 4.84- 4.59 (m, 1H), 4.49-4.10 (m, 3H), 4.03-3.58 (m, 7H), 2.50-2.37 (m, 1H), 2.30-2.21 (m, 4H), 2.16- 1.97 (m, 3H), 1.95- 1.81 (m, 1H). |
| | ic acid | or | | |
| | | 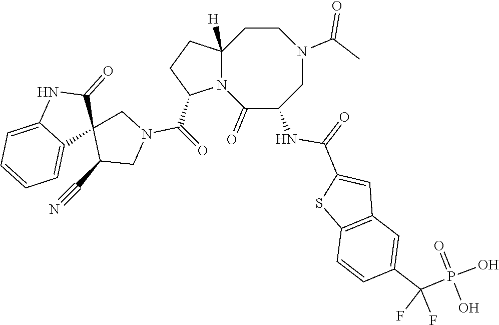 [SMILES: CC(=O)N1CC[C@H]2CC[C@@H](C(=O)N3C[C@@H](C#N)[C@]4(C3)C(=O)Nc3ccccc34)N2C(=O)[C@@H](NC(=O)c2cc3cc(C(F)(F)P(=O)(O)O)ccc3s2)C1] | | |
| | | SFC peak 1 was used for | | |
| | | biological testing | | |
| 14 | ((7- (((5S,8S,10aR)- 3-acetyl- 8- (methyl(phe- nyl)carbamoyl)- 6- oxodecahydro- pyrrolo[1,2- a][1,5]diazocin- 5- yl)carbamoyl) naphthalen- 2- yl)fluorometh- yl)phosphon- ic acid | 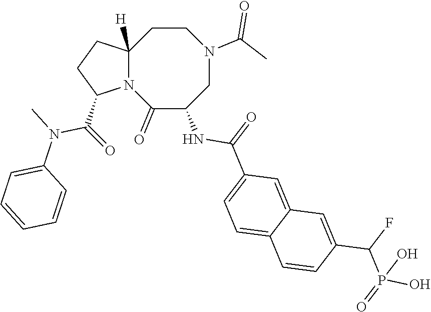 [SMILES: CC(=O)N1CC[C@H]2CC[C@@H](C(=O)N(C)c3ccccc3)N2C(=O)[C@@H](NC(=O)c2ccc3ccc(C(F)P(=O)(O)O)cc3c2)C1] | 625.4 [M + H]⁺ | ¹H NMR (400 MHz, METHANOL-d₄) δ 8.60- 8.35 (m, 1H), 8.19- 8.04 (m, 1H), 8.02-7.84 (m, 3H), 7.75 (d, J = 8.1 Hz, 1H), 7.55-7.24 (m, 5H), 6.08-5.76 (m, 1H), 5.14-4.97 (m, 1H), 4.49- 4.25 (m, 2H), 4.24- 3.99 (m, 1H), 3.93-3.81 (m, 1H), 3.77-3.44 (m, 2H), 3.30-3.20 (m, 3H), 2.39-2.25 (m, 3H), 2.23- 1.72 (m, 6H). |
| 15 | ((7- (((5S,8S,10aR)- 3-acetyl- 8- (methyl(phe- nyl)carbamoyl)- 6- oxodecahydro- pyrrolo[1,2- a][1,5]diazocin- 5- yl)carbamoyl) naphthalen- 2- yl)methyl)phos- phonic acid | 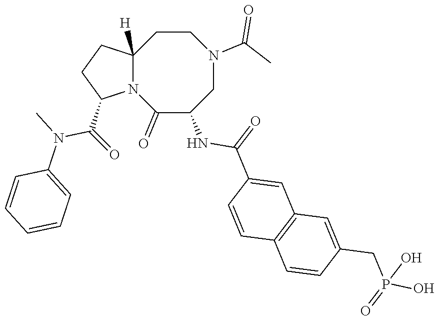 [SMILES: CC(=O)N1CC[C@H]2CC[C@@H](C(=O)N(C)c3ccccc3)N2C(=O)[C@@H](NC(=O)c2ccc3ccc(CP(=O)(O)O)cc3c2)C1] | 607.3 [M + H]⁺ | ¹H NMR (400 MHz, METHANOL-d₄) δ 8.49- 8.36 (m, 1H), 7.99- 7.83 (m, 4H), 7.67-7.55 (m, 1H), 7.53-7.26 (m, 5H), 5.15-4.97 (m, 1H), 4.47-4.24 (m, 2H), 4.22- 3.99 (m, 1H), 3.94- 3.80 (m, 1H), 3.78-3.50 (m, 2H), 3.43-3.32 (m, 2H), 3.30-3.20 (m, 3H), 2.43-2.23 (m, 3H), 2.23- 1.67 (m, 6H). |
| 16 | ((2- (((5S,8S,10aR)- 3-acetyl- 8- (methyl(phe- nyl)carbamoyl)- 6- oxodecahydro- pyrrolo[1,2- a][1,5]diazocin- 5- yl)carbamoyl) benzo[b]thio- phen-5- yl)(cyano)meth- yl)phosphon- ic acid | 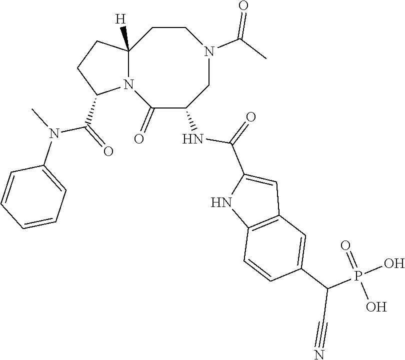 [SMILES: CC(=O)N1CC[C@H]2CC[C@@H](C(=O)N(C)c3ccccc3)N2C(=O)[C@@H](NC(=O)c2cc3cc(C(C#N)P(=O)(O)O)ccc3[nH]2)C1] | 638.1 [M + H]⁺ | ¹H NMR (400 MHz, METHANOL-d₄) δ 8.11- 7.89 (m, 3H), 7.61- 7.55 (m, 1H), 7.51-7.33 (m, 5H), 5.07-4.90 (m, 1H), 4.70-4.59 (m, 1H), 4.44-4.36 (m, 1H), 4.31- 4.12 (m, 1H), 4.01 (dd, J = 14.2, 3.4 Hz, 1H), 3.89-3.78 (m, 1H), 3.76- 3.50 (m, 2H), 3.28- 3.18 (m, 3H), 2.34-2.23 (m, 3H), 2.13-1.67 (m, 6H). |
| 17 | ((2- (((5S,8S,10aR)- 8-([1,1′- biphenyl]-4- ylcarbamoyl)- 3-acetyl-6- oxodecahydro- pyrrolo[1,2- a][1,5]diazocin- 5- yl)carbamoyl) benzo[b]thio- phen-5- yl)fluorometh- yl)phosphon- ic acid | 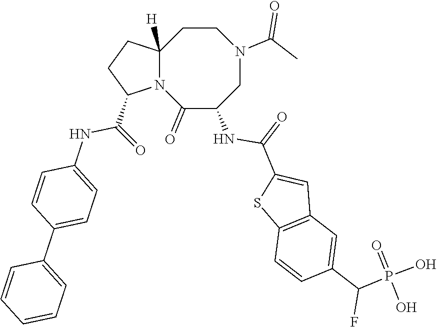 [SMILES: CC(=O)N1CC[C@H]2CC[C@@H](C(=O)Nc3ccc(-c4ccccc4)cc3)N2C(=O)[C@@H](NC(=O)c2cc3cc(C(F)P(=O)(O)O)ccc3s2)C1] | 693.3 [M + H]⁺ | ¹H NMR (400 MHz, METHANOL-d₄) δ 8.08- 7.94 (m, 2H), 7.90- 7.80 (m, 1H), 7.74-7.51 (m, 7H), 7.47-7.37 (m, 2H), 7.33-7.24 (m, 1H), 5.80-5.56 (m, 1H), 5.25- 5.06 (m, 1H), 4.69- 4.56 (m, 1H), 4.51-4.36 (m, 1H), 4.01-3.54 (m, 4H), 2.43-2.32 (m, 1H), 2.27-1.84 (m, 8H). |
| 18 | (1-(2- (((5S,8S,10aR)- 3-acetyl- 8- (methyl(phe- nyl)carbamoyl)- 6- oxodecahydro- pyrrolo[1,2- a][1,5]diazocin- 5- yl)carbamoyl) benzo[b]thio- phen-5-yl)-1- fluoro- ethyl)phos- phonic acid | 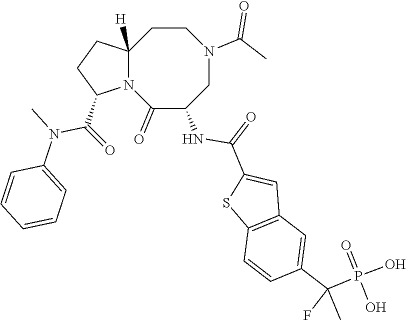 [SMILES: CC(=O)N1CC[C@H]2CC[C@@H](C(=O)N(C)c3ccccc3)N2C(=O)[C@@H](NC(=O)c2cc3cc(C(C)(F)P(=O)(O)O)ccc3s2)C1] | 645.3 [M + H]⁺ | ¹H NMR (400 MHz, METHANOL-d₄) δ 8.13- 7.99 (m, 2H), 7.96- 7.85 (m, 1H), 7.70-7.58 (m, 1H), 7.52-7.34 (m, 5H), 5.08-4.91 (m, 1H), 4.47-4.22 (m, 2H), 4.21- 3.97 (m, 1H), 3.91- 3.78 (m, 1H), 3.74-3.48 (m, 2H), 3.29-3.20 (m, 3H), 2.37-2.23 (m, 3H), 2.20-1.66 (m, 9H). |
| 19 | (1-(2- (((5S,8S,10aR)- 3-acetyl- 8- (methyl(phe- nyl)carbamoyl)- 6- oxodecahydro- pyrrolo[1,2- a][1,5]diazocin- 5- yl)carbamoyl) benzo[b]thio- phen-5-yl)-2- hydroxyethyl) phosphonic acid | 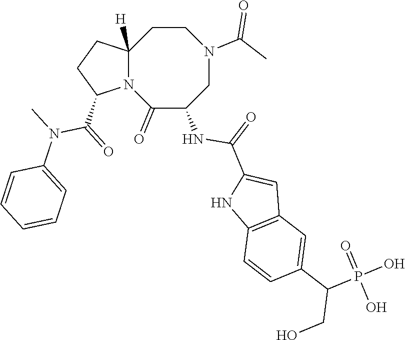 [SMILES: CC(=O)N1CC[C@H]2CC[C@@H](C(=O)N(C)c3ccccc3)N2C(=O)[C@@H](NC(=O)c2cc3cc(C(CO)P(=O)(O)O)ccc3[nH]2)C1] | 643.2 [M + H]⁺ | ¹H NMR (400 MHz, METHANOL-d₄) δ 8.11- 8.00 (m, 1H), 7.98- 7.81 (m, 2H), 7.69-7.20 (m, 6H), 5.08-4.90 (m, 1H), 4.47-4.36 (m, 1H), 4.32-4.23 (m, 1H), 4.21- 4.07 (m, 1H), 4.06- 3.95 (m, 1H), 3.89-3.78 (m, 1H), 3.76-3.59 (m, 1H), 3.58-3.49 (m, 1H), 3.28-3.11 (m, 3H), 2.40- 2.24 (m, 3H), 2.11- 1.67 (m, 6H), 1.45-1.31 (m, 2H). |
| 20 | (1-(2- (((5S,8S,10aR)- 3-acetyl- 8- (methyl(phe- nyl)carbamoyl)- 6- oxodecahydro- pyrrolo[1,2- a][1,5]diazocin- 5- yl)carbamoyl) benzo[b]thio- phen-5- yl)ethyl)phos- phonic acid | 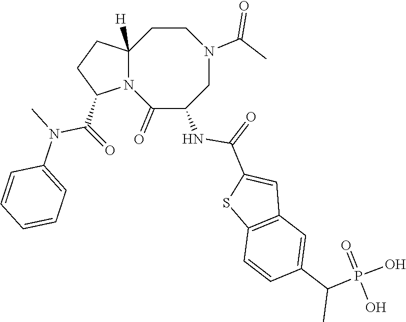 [SMILES: CC(=O)N1CC[C@H]2CC[C@@H](C(=O)N(C)c3ccccc3)N2C(=O)[C@@H](NC(=O)c2cc3cc(C(C)P(=O)(O)O)ccc3s2)C1] | 627.2 [M + H]⁺ | ¹H NMR (400 MHz, METHANOL-d₄) δ 8.09- 8.00 (m, 1H), 7.96- 7.78 (m, 2H), 7.56-7.22 (m, 6H), 5.07-4.91 (m, 1H), 4.49-4.22 (m, 2H), 4.19-3.98 (m, 1H), 3.89- 3.78 (m, 1H), 3.70- 3.50 (m, 2H), 3.29-3.21 (m, 4H), 2.38-2.24 (m, 3H), 2.04-1.72 (m, 6H), 1.70-1.60 (m, 3H). |
| 21 | (2-(2- (((5S,8S,10aR)- 3-acetyl- 8- (methyl(phe- nyl)carbamoyl)- 6- oxodecahydro- pyrrolo[1,2- a] [1,5]diazocin- 5- yl)carbamoyl) benzo[b]thio- phen-5- yl)propan-2- yl)phospnoic aicd | 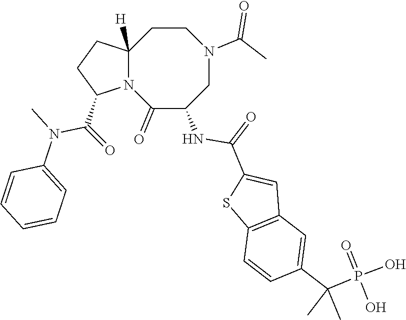 [SMILES: CC(=O)N1CC[C@H]2CC[C@@H](C(=O)N(C)c3ccccc3)N2C(=O)[C@@H](NC(=O)c2cc3cc(C(C)(C)P(=O)(O)O)ccc3s2)C1] | 641.2 [M + H]⁺ | ¹H NMR (400 MHz, METHANOL-d₄) δ 8.14- 7.59 (m, 4H), 7.52- 7.33 (m, 5H), 5.10-4.93 (m, 1H), 4.44-4.31 (m, 1H), 4.30-4.12 (m,1H), 4.09-3.97 (m, 1H), 3.89- 3.79 (m, 1H), 3.76- 3.59 (m, 1H), 3.58-3.46 (m, 1H), 3.30-3.22 (m, 3H), 2.41-2.22 (m, 3H), 2.08-1.87 (m, 5H), 1.82- 1.53 (m, 7H). |
| 22 | ((2- (((5S,8S,10aR)- 3-acetyl- 8- (methyl(phe- nyl)carbamoyl)- 6- oxodecahydro- pyrrolo[1,2- a][1,5]diazocin- 5- yl)carbamoyl) benzo[b]thio- phen-5- yl)fluorometh- yl)phosphon- ic acid | 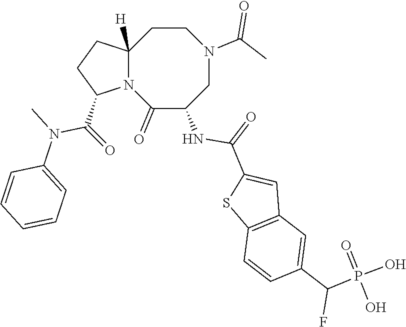 [SMILES: CC(=O)N1CC[C@H]2CC[C@@H](C(=O)N(C)c3ccccc3)N2C(=O)[C@@H](NC(=O)c2cc3cc(C(F)P(=O)(O)O)ccc3s2)C1] | 631.2 [M + H]⁺ | ¹H NMR (400 MHz, METHANOL-d₄) δ 8.13- 7.89 (m, 3H), 7.67- 7.55 (m, 1H), 7.53-7.32 (m, 5H), 5.93-5.74 (m, 1H), 5.08-4.91 (m, 1H), 4.48-4.22 (m, 2H), 4.21- 3.97 (m, 1H), 3.93- 3.78 (m, 1H), 3.76-3.49 (m, 2H), 3.29-3.21 (m, 3H), 2.37-2.24 (m, 3H), 2.23-1.69 (m, 6H). |
| 23 | ((2- (((5S,8S,10aR)- 3-acetyl- 8-((R)-3-(N- methylpropion- amido)pyrro- lidine-1- carbonyl)-6- oxodecahydro- pyrrolo[1,2- a][1,5]diazocin- 5- yl)carbamoyl)- 1H-indol-5- yl)difluorometh- yl)phosphon- ic acid | 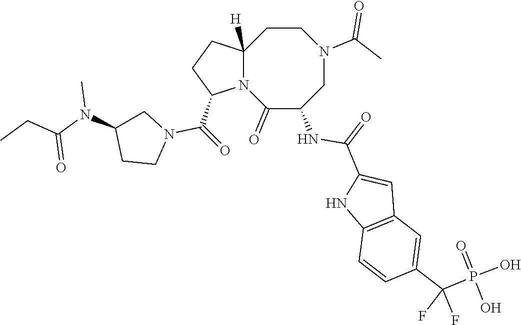 | 631.2 [M + H]⁺ | ¹H NMR (400 MHz, METHANOL-d₄) δ 7.93- 7.90 (m, 1H), 7.55- 7.46 (m, 2H), 7.30-7.20 (m, 1H), 5.30-4.96 (m, 2H), 4.79-4.63 (m, 1H), 4.49-4.31 (m, 1H), 4.19- 3.94 (m, 1H), 3.89- 3.32 (m, 7H), 3.03-2.80 (m, 3H), 2.57-2.33 (m, 3H), 2.31 (s, 2H), 2.26- 2.12 (m, 3H), 2.10-1.81 (m, 5H), 1.36-0.98 (m, 3H). |
| 24 | ((2- (((5S,8S,10aR)- 3-acetyl- 8-((1- acetylazetidin- 3- yl)carbamoyl)- 6- oxodecahydro- pyrrolo[1,2- a][1,5]diazocin- 5- yl)carbamoyl)- 1H-indol-5- yl)difluorometh- yl)phosphon- ic acid | 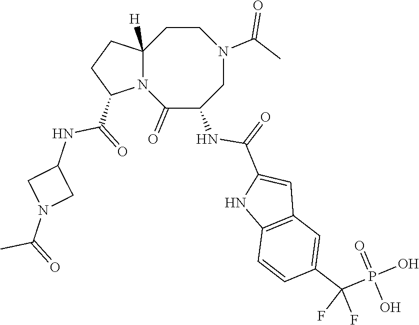 [SMILES: CC(=O)N1CC(NC(=O)[C@@H]2CC[C@@H]3CCN(C(C)=O)C[C@H](NC(=O)c4cc5cc(C(F)(F)P(=O)(O)O)ccc5[nH]4)C(=O)N32)C1] | 639.3 [M + H]⁺ | ¹H NMR (400 MHz, METHANOL-d₄) δ 7.93 (br. s., 1H), 7.61-7.42 (m, 2H), 7.32-7.16 (m, 1H), 5.24-5.05 (m, 1H), 4.91-4.87 (m, 1H), 4.60- 4.01 (m, 6H), 3.99- 3.53 (m, 5H), 2.42-1.94 (m, 7H), 1.93-1.70 (m, 4H). |
| 25 | ((4-((E)-4- (((5S,8S,10aR)- 3-acetyl- 8-(methyl(3- propionamido- propyl)carba- moyl)-6- oxodecahydro- pyrrolo[1,2- a][1,5]diazocin- 5- yl)amino)-4- oxobut-2-en- 2- yl)phenyl)diflu- oromethyl) phosphonic acid | 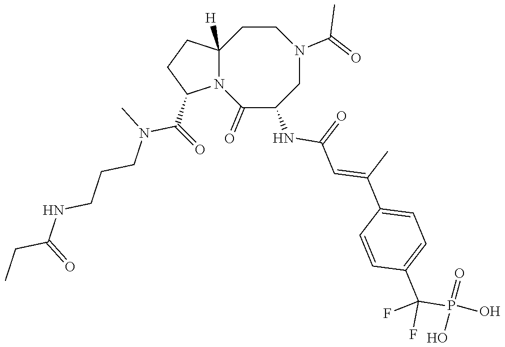 [SMILES: CCC(=O)NCCCN(C)C(=O)[C@@H]1CC[C@@H]2CCN(C(C)=O)C[C@H](NC(=O)/C=C(\C)c3ccc(C(F)(F)P(=O)(O)O)cc3)C(=O)N21] | 670.3 [M + H]⁺ | ¹H NMR (400 MHz, METHANOL-d₄) δ 7.67 (d, J = 8.1 Hz, 2H), 7.54 (d, J = 8.3 Hz, 2H), 6.39- 6.25 (m, 1H), 5.02- 4.94 (m, 1H), 4.92-4.87 (m, 1H), 4.50-4.29 (m, 1H), 4.08-3.34 (m, 6H), 3.25-2.82 (m, 5H), 2.55- 2.50 (m, 3H), 2.46- 2.34 (m, 1H), 2.31-2.10 (m, 6H), 2.08-1.61 (m, 6H), 1.21-1.02 (m, 3H). |
| 26 | ((4-((E)-3- (((5S,8S,10aR)- 3-acetyl- 8-(methyl(3- propionamido- propyl)carba- moyl)-6- oxodecahydro- pyrrolo[1,2- a][1,5]diazocin- 5- yl)amino)-3- oxoprop-1- en-1- yl)phenyl)diflu- oromethyl) phosphonic acid | 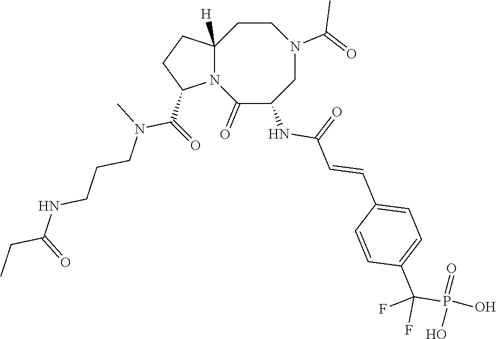 [SMILES: CCC(=O)NCCCN(C)C(=O)[C@@H]1CC[C@@H]2CCN(C(C)=O)C[C@H](NC(=O)/C=C/c3ccc(C(F)(F)P(=O)(O)O)cc3)C(=O)N21] | 656.3 [M + H]⁺ | ¹H NMR (400 MHz, METHANOL-d₄) δ 7.73- 7.54 (m, 5H), 6.89- 6.74 (m, 1H), 5.05-4.91 (m, 1H), 4.84-4.82 (m, 1H), 4.48-4.28 (m, 1H), 4.10-3.33 (m, 6H), 3.27- 3.04 (m, 4H), 2.94- 2.89 (m, 1H), 2.45-2.34 (m, 1H), 2.33-2.13 (m, 6H), 2.12-2.00 (m, 1H), 1.99-1.66 (m, 5H), 1.16- 1.05 (m, 3H). |
| 27 | (2- (((5S,8S,10aR)- 3-acetyl- 8-(methyl(3- propionamido- propyl)carba- moyl)-6- oxodecahydro- pyrrolo[1,2- a][1,5]diazocin- 5- yl)carbamoyl)- 1H-indole- 5- carbonyl)phos- phonic acid | 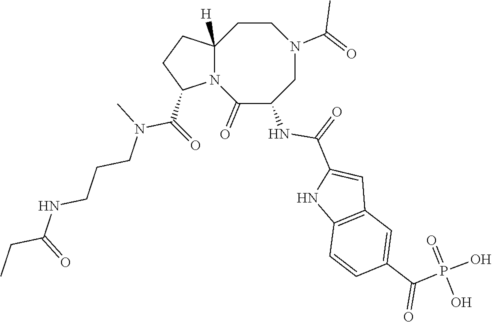 [SMILES: CCC(=O)NCCCN(C)C(=O)[C@@H]1CC[C@@H]2CCN(C(C)=O)C[C@H](NC(=O)c3cc4cc(C(=O)P(=O)(O)O)ccc4[nH]3)C(=O)N21] | 647.2 [M + H]⁺ | ¹H NMR (400 MHz, METHANOL-d₄) δ 8.94- 8.74 (m, 1H), 8.13- 8.00 (m, 1H), 7.61-7.48 (m, 1H), 7.45-7.31 (m, 1H), 5.19-5.06 (m, 1H), 4.95-4.88 (m, 1H), 4.53- 4.32 (m, 1H), 4.28- 3.70 (m, 3H), 3.69-3.36 (m, 3H), 3.27-2.87 (m, 5H), 2.50-1.64 (m, 13H), 1.22-0.92 (m, 3H). |
| 28 | ((2- (((5S,8S,10aR)- 3-acetyl- 8-(methyl(3- propionamido- propyl)carba- moyl)-6- oxodecahydro- pyrrolo[1,2- a][1,5]diazocin- 5- yl)carbamoyl)- 1H-indol-5- yl)difluorometh- yl)phosphon- ic acid | 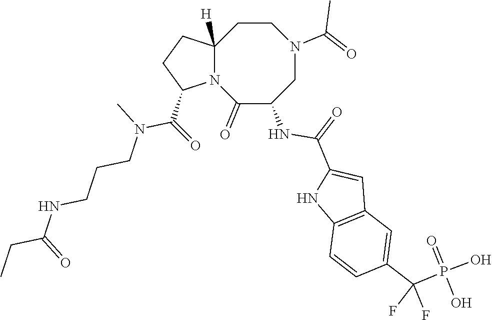 [SMILES: CCC(=O)NCCCN(C)C(=O)[C@@H]1CC[C@@H]2CCN(C(C)=O)C[C@H](NC(=O)c3cc4cc(C(F)(F)P(=O)(O)O)ccc4[nH]3)C(=O)N21] | 669.3 [M + H]⁺ | ¹H NMR (400 MHz, METHANOL-d₄) δ 7.92 (s, 1H), 7.54-7.45 (m, 2H), 7.30-7.19 (m, 1H), 5.16-4.98 (m, 1H), 4.93- 4.87 (m, 1H), 4.49- 4.33 (m, 1H), 4.06-3.37 (m, 5H), 3.26-3.07 (m, 4H), 2.96-2.89 (m, 1H), 2.45-1.69 (m, 14H), 1.15-0.98 (m, 3H). |
| 29 | ((2- (((5S,8S,10aR)- 3-acetyl- 8- (dimethylcarba- moyl)-6- oxodecahydro- pyrrolo[1,2- a][1,5]diazocin- 5- yl)carbamoyl)- 1H-indole-5- yl)difluorometh- yl)phosphon- ic acid | 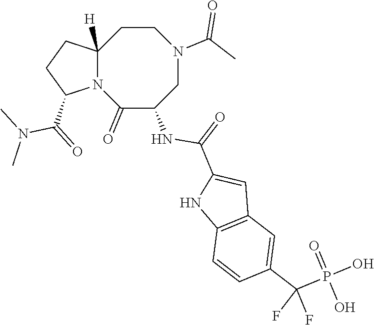 [SMILES: CC(=O)N1CC[C@H]2CC[C@@H](C(=O)N(C)C)N2C(=O)[C@@H](NC(=O)c2cc3cc(C(F)(F)P(=O)(O)O)ccc3[nH]2)C1] | 570.2 [M + H]⁺ | ¹H NMR (400 MHz, METHANOL-d₄) δ 7.92 (s, 1H), 7.60-7.41 (m, 2H), 7.33-7.15 (m, 1H), 5.14-4.98 (m, 1H), 4.95- 4.89 (m, 1H), 4.46- 4.30 (m, 1H), 4.29-3.97 (m, 1H), 3.95-3.79 (m, 1H), 3.65-3.41 (m, 2H), 3.21-3.11 (m, 3H), 3.02- 2.89 (m, 3H), 2.51- 2.03 (m, 6H), 2.01-1.79 (m, 3H). |
| 30 | ((2- (((5S,8S,10aR)- 3-acetyl- 8- (methyl(phenyl carbamoyl)- 6- oxodecahydro- pyrrolo[1,2- a][1,5]diazocin- 5- yl)carbamoyl)- 1H-indol-5- yl)difluorometh- yl)phosphon- ic acid | 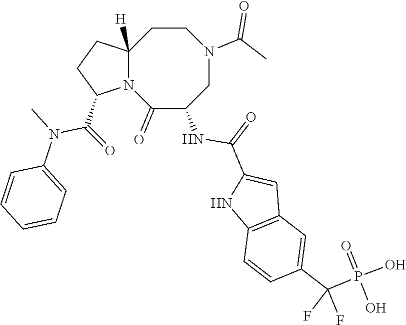 [SMILES: CC(=O)N1CC[C@H]2CC[C@@H](C(=O)N(C)c3ccccc3)N2C(=O)[C@@H](NC(=O)c2cc3cc(C(F)(F)P(=O)(O)O)ccc3[nH]2)C1] | 632.2 [M + H]⁺ | ¹H NMR (400 MHz, METHANOL-d₄) δ 7.96- 7.89 (m, 1H), 7.53- 7.34 (m, 7H), 7.31-7.23 (m, 1H), 5.12-4.95 (m, 1H), 4.46-4.14 (m, 2H), 4.05-3.78 (m, 2H), 3.72- 3.45 (m, 2H), 3.28- 3.21 (m, 3H), 2.35-2.23 (m, 3H), 2.20-1.69 (m, 6H). |
| 31 | ((2- (((5S,8S,10aR)- 3-acetyl- 8-(((1S,2R)- 2- aminocyclopent- yl)carbamoyl)- 6- oxodecahydro- pyrrolo[1,2- a][1,5]diazocin- 5- yl)carbamoyl)- 1H-indol-5- yl)difluorometh- yl)phosphon- ic acid | 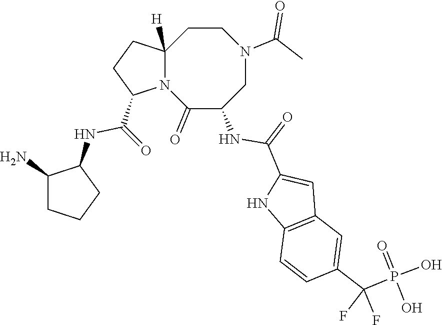 [SMILES: CC(=O)N1CC[C@H]2CC[C@@H](C(=O)N[C@H]3CCC[C@H]3N)N2C(=O)[C@@H](NC(=O)c2cc3cc(C(F)(F)P(=O)(O)O)ccc3[nH]2)C1] | 625.2 [M + H]⁺ | ¹H NMR (400 MHz, CD₃CN:D₂O 1:1) δ 7.87 (s, 1H), 7.51-7.44 (m, 2H), 7.27-7.21 (m, 1H), 5.14-5.10 (m, 1H), 4.33- 4.29 (m, 2H), 4.17- 4.01 (m, 1H), 3.80-3.39 (m, 5H), 2.25-1.98 (m, 9H), 1.85-1.71 (m, 3H), 1.70-1.49 (m, 3H). |
| 32 | ((2- (((5S,8S,10aR)- 8- (((1S,2S)-2- acetamidocy- clopentyl)car- bamoyl)-3- acetyl-6- oxodecahydro- pyrrolo[1,2- a][1,5]diazocin- 5- yl)carbamoyl)- 1H-indol-5- yl)difluorometh- yl)phosphon- ic acid | 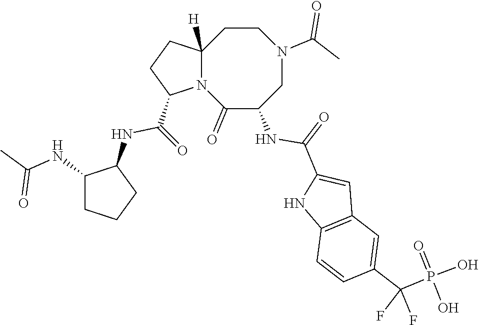 [SMILES: CC(=O)N[C@H]1CCC[C@@H]1NC(=O)[C@@H]1CC[C@@H]2CCN(C(C)=O)C[C@H](NC(=O)c3cc4cc(C(F)(F)P(=O)(O)O)ccc4[nH]3)C(=O)N21] | 667.3 [M + H]⁺ | ¹H NMR (400 MHz, METHANOL-d₄) δ 7.92 (s, 1H), 7.54-7.46 (m, 2H), 7.29-7.22 (m, 1H), 5.16-5.04 (m, 1H), 4.44- 4.30 (m, 2H), 4.10- 3.93 (m, 3H), 3.83-3.50 (m, 3H), 2.32-2.21 (m, 4H), 2.17-1.86 (m, 9H), 1.84-1.69 (m, 3H), 1.63- 1.44 (m, 2H). |
| 33 | ((2- (((5S,8S,10aR)- 3-acetyl- 8-((3- (ethyl(methyl) amino)propyl) (methyl)car- bamoyl)-6- oxodecahydro- pyrrolo[1,2- a][1,5]diazocin- 5- yl)carbamoyl)- 1H-indol-5- yl)difluorometh- yl)phosphon- ic acid | 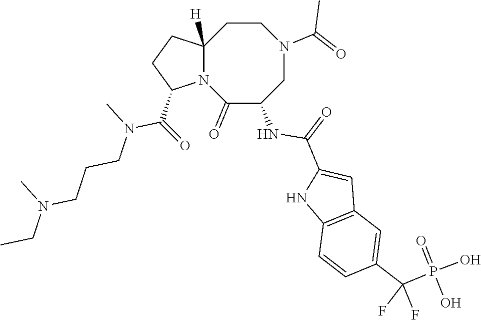 [SMILES: CCN(C)CCCN(C)C(=O)[C@@H]1CC[C@@H]2CCN(C(C)=O)C[C@H](NC(=O)c3cc4cc(C(F)(F)P(=O)(O)O)ccc4[nH]3)C(=O)N21] | 655.3 [M + H]⁺ | ¹H NMR (400 MHz, CD₃CN:D₂O 1:1) δ 7.87 (s, 1H), 7.53-7.43 (m, 2H), 7.23-7.16 (m, 1H), 5.03-5.01 (m, 1H), 4.76- 4.64 (m, 1H), 4.41- 4.28 (m, 1H), 3.88-3.85 (m, 1H), 3.77-3.53 (m, 3H), 3.47-3.42 (m, 1H), 3.33-2.92 (m, 9H), 2.86- 2.60 (m, 4H), 2.34- 2.28 (m, 1H), 2.19-2.07 (m, 4H), 1.84-1.71 (m, 3H), 1.26-1.15 (m, 3H), 1.12-0.99 (m, 1H). |
| 34 | ((2- (((5S,8S,10aR)- 3-aceytl- 6-oxo-8- (((R)-1- propionylpyr- rolidin-3- yl)carbamoyl) decahydropyr- rolo[1,2- a][1,5]diazocin- 5- yl)carbamoyl)- 1H-indol-5- yl)difluorometh- yl)phosphon- ic acid | 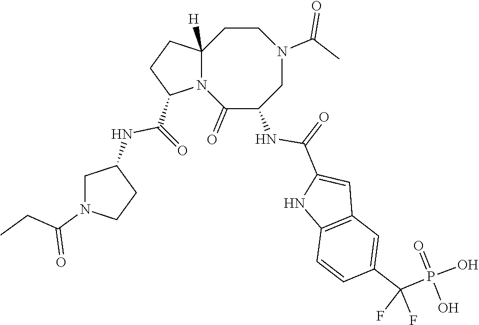 [SMILES: CCC(=O)N1CC[C@@H](NC(=O)[C@@H]2CC[C@@H]3CCN(C(C)=O)C[C@H](NC(=O)c4cc5cc(C(F)(F)P(=O)(O)O)ccc5[nH]4)C(=O)N32)C1] | 667.3 [M + H]⁺ | ¹H NMR (400 MHz, 1:1 CD₃CN:D₂O) δ 7.87 (s, 1H), 7.56-7.39 (m, 2H), 7.26-7.16 (m, 1H), 5.13- 4.98 (m, 1H), 4.37- 4.22 (m, 3H), 3.86-3.22 (m, 8H), 2.32-2.02 (m, 8H), 1.87-1.69 (m, 4H), 1.29-0.93 (m, 4H). |
| 35 | ((2- (((5S,8S,10aR)- 3-acetyl- 8-(4- ((benzyloxy) carbonyl)piper- azine-1- carbonyl)-6- oxodecahydro- pyrrolo[1,2- a][1,5]diazocin- 5- yl)carbamoyl)- 1H-indol-5- yl)difluorometh- yl)phosphon- ic acid | 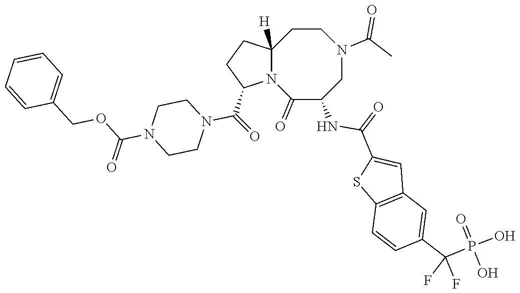 | 745.3 [M + H]⁺ | ¹H NMR (400 MHz, 1:1 CD₃CN:D₂O) δ 7.85 (s, 1H), 7.53-7.40 (m, 2H), 7.37-7.25 (m, 5H), 7.20 (s, 1H), 5.09-5.04 (m, 2H), 5.04-4.92 (m, 1H), 4.82-4.70 (m, 1H), 4.36- 4.22 (m, 1H), 4.04- 3.83 (m, 1H), 3.75-3.64 (m, 1H), 3.62-3.31 (m, 10H), 2.33-2.21 (m, 1H), 2.19-1.98 (m, 4H), 1.90-1.63 (m, 4H). |
| 36 | ((2- (((5S,8S,10aR)- 3-acetyl- 8- (methylcarba- moyl)-6- oxodecahydro- pyrrolo[1,2- a][1,5]diazocin- 5- yl)carbamoyl)- 1H-indol-5- yl)difluorometh- yl)phosphon- ic acid | 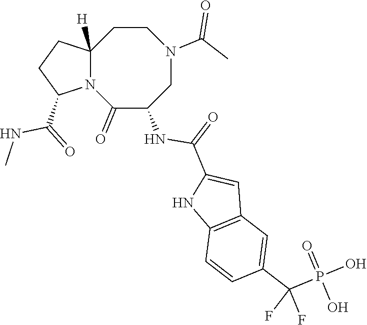 [SMILES: CNC(=O)[C@@H]1CC[C@@H]2CCN(C(C)=O)C[C@H](NC(=O)c3cc4cc(C(F)(F)P(=O)(O)O)ccc4[nH]3)C(=O)N21] | 556.2 [M + H]⁺ | ¹H NMR (400 MHz, 1:1 CD₃CN:D₂O) δ 7.87 (s, 1H), 7.55-7.38 (m, 2H), 7.23 (s, 1H), 5.15-4.99 (m, 1H), 4.38-4.24 (m, 2H), 3.88-3.77 (m, 1H), 3.75-3.41 (m, 3H), 2.70- 2.52 (m, 3H), 2.26- 1.95 (m, 6H), 1.89-1.66 (m, 3H). |
| 37 | ((7- (((5S,8S,10aR)- 3-acetyl- 6-oxo-8-(6- (2-oxo-1,2- dihydropyridin- 4-yl)-4- azaspiro[2.4] heptane-4- carbonyl)deca- hydropyrrolo [1,2- a][1,5]diazocin- 5- yl)carbamoyl) naphthalen- 2- yl)difluorometh- yl)phosphon- | 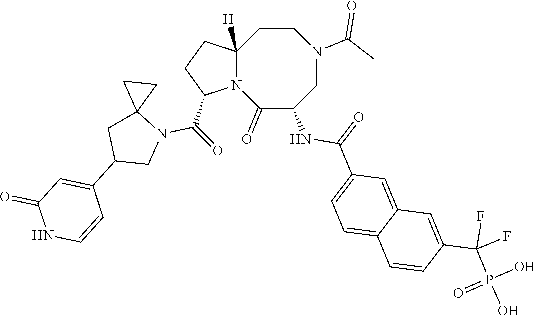 [SMILES: CC(=O)N1CC[C@H]2CC[C@@H](C(=O)N3CC(c4cc[nH]c(=O)c4)CC34CC4)N2C(=O)[C@@H](NC(=O)c2ccc3ccc(C(F)(F)P(=O)(O)O)cc3c2)C1] | 726.3 [M + H]⁺ | ¹H NMR (400 MHz, DMSO-d₆) δ 8.97-8.72 (m, 1H), 8.64-8.53 (m, 2H), 8.35-8.12 (m, 1H), 8.03-7.88 (m, 3H), 7.79-7.72 (m, 1H), 7.33-7.24 (m, 1H), 6.40-6.13 (m, 2H), 5.12-4.86 (m, 1H), 4.66-4.50 (m, 1H), 4.32-4.20 (m, 1H), 4.05-3.95 (m, 1H), 3.94-3.83 (m, 1H), 3.72-3.62 (m, 1H), 3.59-3.50 (m, 2H), 3.12-2.96 (m, 2H), 2.33-2.23 (m, 1H), 2.22-2.12 (m, 3H), 2.10-2.06 (m, |
| | ic acid | | | 1H), 2.02-1.92 (m, |
| | | | | 3H), 1.78-1.68 (m, |
| | | | | 2H), 1.64-1.56 (m, |
| | | | | 1H), 1.17-1.15 (m, |
| | | | | 2H), 0.53-0.38 (m, |
| | | | | 2H). |
| 38 | ((7- (((5S,8S,10aR)- 3-acetyl- 8-((3R,4S or 3S,4R)-3- cyano-4-(2- oxo-1,2- dihydropyridin- 4- yl)pyrrolidine- 1- carbonyl)-6- oxodecahydro- pyrrolo[1,2- a][1,5]diazocin- 5- yl)carbamoyl) naphthalen- 2- yl)difluorometh- yl)phosphon- | 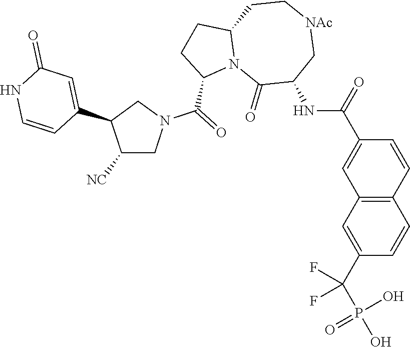 | 725.0 [M + H]⁺ | ¹H NMR (400 MHz, DMSO-d₆) δ 8.90-8.48 (m, 2H), 8.25-8.17 (m, 1H), 8.10-8.01 (m, 4H), 7.74-7.70 (m, 1H), 7.45-7.32 (m, 1H), 6.42-6.33 (m, 1H), 6.30-6.20 (m, 1H), 5.10-4.95 (m, 1H), 4.67-4.57 (m, 1H), 4.33-4.23 (m, 2H), 4.02-3.93 (m, 1H), 3.93-3.81 (m, 2H), 3.79-3.71 (m, 2H), 3.67-3.51 (m, 4H), 3.50-3.38 (m, 2H), 2.34-2.25 (m, 1H), 2.22-2.15 (m, 2H), 2.12-2.05 (m, 3H), 1.26-1.23 (m, 3H). |
| | ic acid | or | | |
| | | 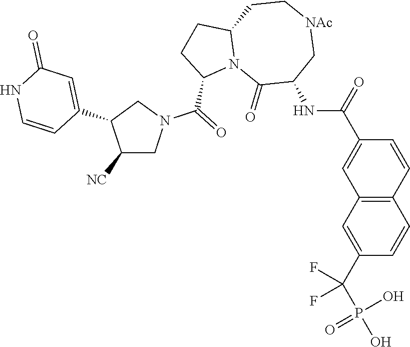 | | |
| | | SFC peak 1 was used for | | |
| | | biological testing | | |
| 39 | ((7- (((5S,8S,10aR)- 3-acetyl- 8-((3S,4R or 3R,4S)-3- cyano-4-(2- oxo-1,2- dihydropyridin- 4- yl)pyrrolidine- 1- carbonyl)-6- oxodecahydro- pyrrolo[1,2- a][1,5]diazocin- 5- yl)carbamoyl) naphthalen- 2- yl)difluorometh- yl)phosphon- | 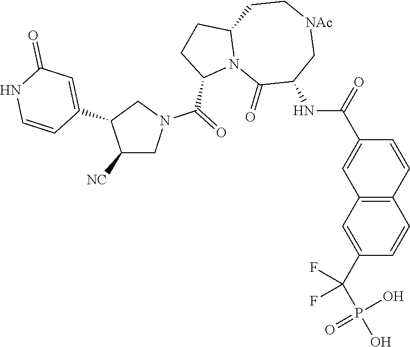 | 725 [M + H]⁺ | ¹H NMR (400 MHz, DMSO-d₆) δ 8.86-8.45 (m, 2H), 8.30-7.97 (m, 4H), 7.78-7.32 (m, 2H), 6.46-6.34 (m, 1H), 6.30-6.18 (m, 1H), 5.10-4.89 (m, 1H), 4.63-4.54 (m, 1H), 4.39-4.28 (m, 1H), 4.21-4.05 (m, 1H), 4.00-3.85 (m, 2H), 3.74-3.60 (m, 2H), 3.54-3.19 (m, 4H), 3.10-3.00 (m, 1H), 2.42-2.26 (m, 1H), 2.25-1.90 (m, 5H), 1.88-1.66 (m, 3H). |
| | ic acid | or | | |
| | | 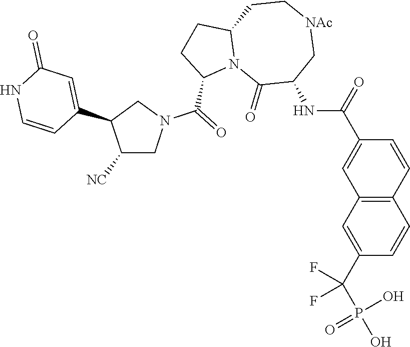 | | |
| | | SFC peak 2 was used for | | |
| | | biological testing | | |
| 40 | ((2- (((5S,8S,10aR)- 2-acetyl- 6-oxo-8-(3- (pyridin-3- yl)azetidine- 1- carbonyl)deca- hydropyrrolo [1,2- a][1,4]diazocin- 5- yl)carbamoyl) benzo[b]thio- phen-5- yl)difluorometh- yl)phosphon- ic acid | 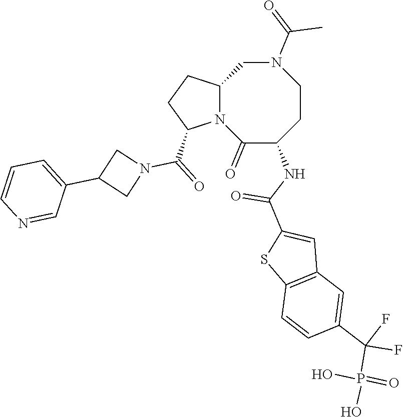 [SMILES: CC(=O)N1CC[C@H](NC(=O)c2cc3cc(C(F)(F)P(=O)(O)O)ccc3s2)C(=O)N2[C@H](CC[C@H]2C(=O)N2CC(c3cccnc3)C2)C1] | 676 [M + H]⁺ | ¹H NMR (400 MHz, DMSO-d₆) δ 9.19-8.97 (m, 1H), 8.64-8.41 (m, 2H), 8.39-8.25 (m, 1H), 8.16-8.06 (m, 2H), 7.97-7.81 (m, 1H), 7.59 (d, J = 8.5 Hz, 1H), 7.51-7.33 (m, 1H), 4.89-4.55 (m, 3H), 4.50-4.21 (m, 5H), 3.95-3.78 (m, 3H), 3.46-3.37 (m, 1H), 2.30-1.75 (m, 9H) |
| 41 | ((2- (((5S,8S,10aR)- 3-acetyl- 8-((rel- trans)-3- cyano-3- methyl-4- phenylpyrroli- dine-1- carbonyl)-6- oxodecahydro- pyrrolo[1,2- a][1,5]diazocin- 5- yl)carbamoyl) benzo[b]thio- | 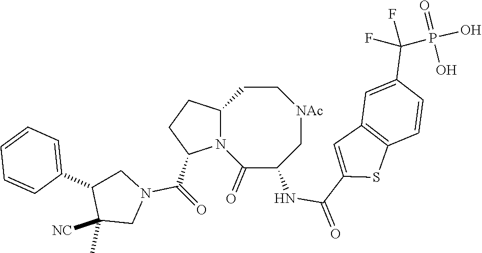 | 769 [M + H]⁺ | ¹H NMR (400 MHz, DMSO-d₆) δ 8.66-8.58 (m, 1H), 8.34-8.28 (m, 1H), 8.16-8.10 (m, 1H), 8.08 (s, 1H), 7.61- 7.56 (m, 1H), 7.34 (s, 3H), 7.29-7.23 (m, 1H), 6.27-5.94 (m, 1H), 4.92-4.85 (m, 1H), 4.62-4.53 (m, 1H), 4.36-4.26 (m, 1H), 4.11-4.04 (m, 1H), 4.01-3.91 (m, 1H), 3.90-3.83 (m, 1H), 3.73-3.63 (m, 2H), 3.56-3.53 (m, |
| | phen-5- | | | 3H), 3.50-3.45(m, |
| | yl)difluorometh- | | | 2H), 3.31-3.25 (m, |
| | yl)phosphon- | | | 2H), 3.16-3.10 (m, |
| | ic acid | | | 3H), 2.35-2.29 (m, |
| | | | | 1H), 2.09-2.00 (m, |
| | | | | 1H), 1.86-1.66 (m, |
| | | | | 4H). |
| 42 | ((2- (((5S,8S,10aR)- 3-acetyl- 8-((2R,4S)-2- benzyl-4- cyanopyrroli- dine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 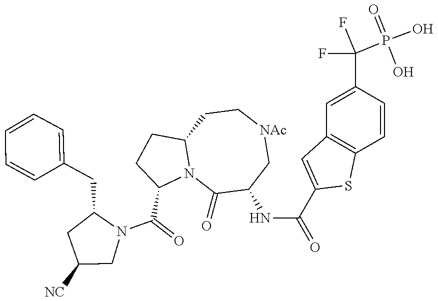 | 728.2 [M + H]⁺ | ¹H NMR (400 MHz, DMSO-d₆) δ 9.20-8.74 (m, 1H), 8.39-8.26 (m, 1H), 8.17-8.03 (m, 2H), 7.59 (d, J = 8.5 Hz, 1H), 7.40-7.15 (m, 5H), 5.09-4.77 (m, 2H), 4.62-4.43 (m, 1H), 4.40-4.15 (m, 2H), 3.99-3.38 (m, 7H), 3.15-2.89 (m, 1H), 2.78-2.55 (m, 1H), 2.47-2.19 (m, 1H), 2.16-2.00 (m, 5H), 1.94-1.49 (m, 4H). |
| 43 | ((2- (((5S,8S,10aR)- 3-acetyl- 8-((2R,4R)- 2-benzyl-4- cyanopyrroli- dine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 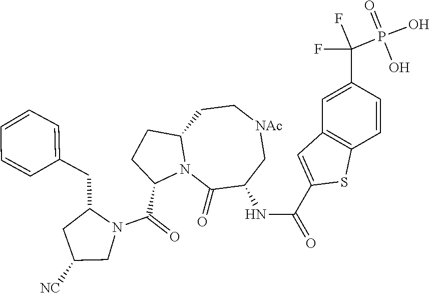 | 728.2 [M + H]⁺ | ¹H NMR (400 MHz, DMSO-d₆) δ 9.24-8.71 (m, 1H), 8.48-8.21 (m, 1H), 8.19-7.95 (m, 2H), 7.74-7.53 (m, 1H), 7.41-7.17 (m, 5H), 5.12-4.72 (m, 2H), 4.62-4.45 (m, 1H), 4.40-4.10 (m, 2H), 4.01-3.71 (m, 3H), 3.63-3.52 (m, 2H), 3.47-3.21 (m, 2H), 3.13-2.59 (m, 2H), 2.39-2.23 (m, 1H), 2.18-1.98 (m, 5H), 1.96-1.72 (m, 4H). |
| 44 | ((2- (((5S,8S,10aR)- 3-acetyl- 8-((3,5- dimethyl- [1,1′- biphenyl]-4- yl)carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 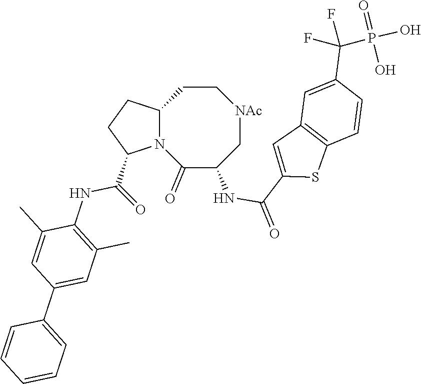 | 739 [M + H]⁺ | ¹H NMR (400 MHz, DMSO-d₆) δ 9.49-9.38 (m, 1H), 9.21-8.72 (m, 1H), 8.41-8.30 (m, 1H), 8.23-8.05 (m, 2H), 7.69-7.25 (m, 8H), 5.12-4.88 (m, 1H), 4.66-4.56 (m, 1H), 4.42-4.28 (m, 1H), 3.89-3.56 (m, 3H), 3.53-3.41 (m, 1H), 2.44-2.30 (m, 1H), 2.29-2.17 (m, 6H), 2.16-2.03 (m, 4H), 2.02-1.71 (m, 4H). |
| 45 | ((3-((E)-3- (((5S,8S,10aR)- 3-acetyl- 8- (methyl(phenyl) carbamoyl)- 6- oxodecahydro- pyrrolo[1,2- a][1,5]diazocin- 5- yl)amino)-2- methyl-3- oxoprop-1- en-1- yl)phenyl)diflu- oromethyl) phosphonic acid | 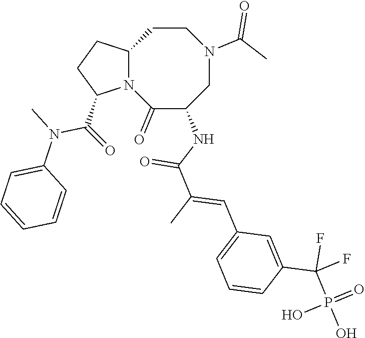 [SMILES: CC(=O)N1CC[C@H]2CC[C@@H](C(=O)N(C)c3ccccc3)N2C(=O)[C@@H](NC(=O)/C(C)=C/c2cccc(C(F)(F)P(=O)(O)O)c2)C1] | 633 [M + H]⁺ | ¹H NMR (400 MHz, DMSO-d₆) δ 8.23-7.84 (m, 1H), 7.60-7.34 (m, 10H), 4.84-4.67 (m, 1H), 4.26-4.15 (m, 2H), 3.83-3.72 (m, 2H), 3.38-3.29 (m, 1H), 3.25-3.10 (m, 4H), 2.22-2.08 (m, 3H), 2.06-1.97 (m, 3H), 1.94-1.51 (m, 6H). |
| 46 | ((2- (((5S,8S,10aR)- 8-([1,1′- biphenyl]-4- ylcarbamoyl)- 3-acetyl-6- oxodecahydro- pyrrolo[1,2- a][1,5]diazocin- 5- yl)carbamoyl)- 6- fluorobenzo[b] thiophen- 5- yl)difluorometh- yl)phosphon- ic acid | 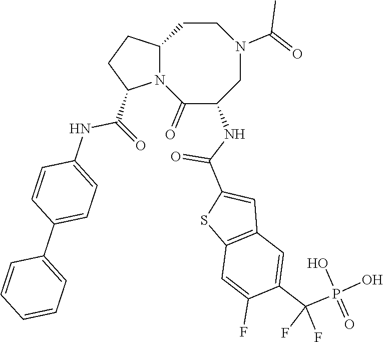 [SMILES: CC(=O)N1CC[C@H]2CC[C@@H](C(=O)Nc3ccc(-c4ccccc4)cc3)N2C(=O)[C@@H](NC(=O)c2cc3cc(C(F)(F)P(=O)(O)O)c(F)cc3s2)C1] | 729.3 [M + H]⁺ | ¹H NMR (400 MHz, DMSO-d₆) δ 10.28- 10.20 (m, 1H), 9.06- 8.67 (m, 1H), 8.31-8.26 (m, 1H), 8.10-8.02 (m, 2H), 7.73-7.60 (m, 6H), 7.47-7.40 (m, 2H), 7.37-7.29 (m, 1H), 5.08-4.82 (m, 1H), 4.58-4.47 (m, 1H), 4.39-4.27 (m, 1H), 3.95-3.82 (m, 1H), 3.76-3.64 (m, 2H), 3.52-3.48 (m, 1H), 2.34-2.25 (m, 1H), 2.16-1.72 (m, 8H). (TFA salt) |
| 47 | ((2- (((5S,8S,10aR)- 3-acetyl- 8-(methyl(4- (3- methyloxetan- 3- yl)phenyl)car- bamoyl)-6- oxodecahydro- pyrrolo[1,2- a][1,5]diazocin- 5- yl)carbamoyl) benzo[b]thio- phen-5- yl)difluorometh- yl)phosphon- ic acid | 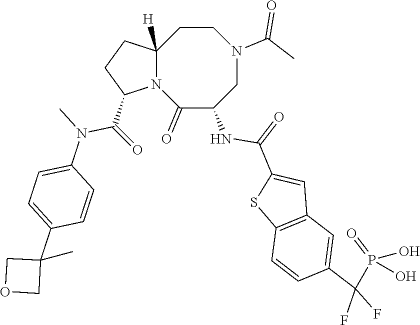 [SMILES: CC(=O)N1CC[C@H]2CC[C@@H](C(=O)N(C)c3ccc(C4(C)COC4)cc3)N2C(=O)[C@@H](NC(=O)c2cc3cc(C(F)(F)P(=O)(O)O)ccc3s2)C1] | 719 [M + H]⁺ | ¹H NMR (400 MHz, DMSO-d₆) δ 9.07-8.59 (m, 1H), 8.38-8.06 (m, 3H), 7.64-7.56 (m, 1H), 7.45-7.21 (m, 4H), 4.99-4.74 (m, 3H), 4.60-4.47 (m, 2H), 4.30-4.14 (m, 2H), 3.89-3.82 (m, 1H), 3.76-3.70 (m, 1H), 3.40-3.28 (m, 2H), 3.18-3.11 (m, 3H), 2.22-1.78 (m, 9H), 1.65-1.61 (m, 3H). |
| 48 | ((2- (((5S,8S,10aR)- 8-([1,1′- biphenyl]-4- ylcarbamoyl)- 3-acetyl-6- oxodecahydro- pyrrolo[1,2- a][1,5]diazocin- 5- yl)carbamoyl)- 4- fluorobeno[b] thiophen- 5- yl)difluorometh- yl)phosphon- ic acid | 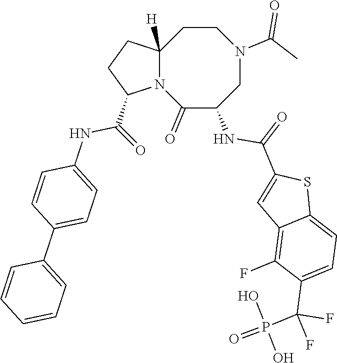 [SMILES: CC(=O)N1CC[C@H]2CC[C@@H](C(=O)Nc3ccc(-c4ccccc4)cc3)N2C(=O)[C@@H](NC(=O)c2cc3c(F)c(C(F)(F)P(=O)(O)O)ccc3s2)C1] | 729.3 [M + H]⁺ | ¹H NMR (400 MHz, DMSO-d₆) δ 10.29- 10.19 (m, 1H), 9.22- 8.94 (m, 1H), 8.48-8.39 (m, 1H), 8.01-7.93 (m, 1H), 7.75-7.59 (m, 6H), 7.55-7.40 (m, 3H), 7.36-7.28 (m, 1H), 5.04-4.90 (m, 1H), 4.54-4.49 (m, 1H), 4.38-4.33 (m, 1H), 3.96-3.75 (m, 2H), 3.69-3.60 (m, 1H), 3.56-3.46 (m, 1H), 2.35-2.22 (m, 1H), 2.14-1.76 (m, 8H). (TFA salt) |
| 49 | ((2- (((5S,8S,10aR)- 3-acetyl- 8- (benzo[d]iso- xazol-5- yl(methyl)car- bamoyl)-6- oxodecahydro- pyrrolo[1,2- a][1,5]diazocin- 5- yl)carbamoyl) benzo[b]thio- phen-5- yl)difluorometh- yl)phosphon- ic acid | 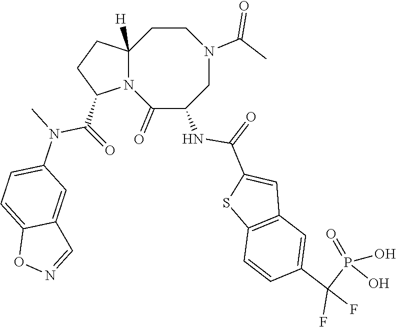 [SMILES: CC(=O)N1CC[C@H]2CC[C@@H](C(=O)N(C)c3ccc4oncc4c3)N2C(=O)[C@@H](NC(=O)c2cc3cc(C(F)(F)P(=O)(O)O)ccc3s2)C1] | 690 [M + H]⁺ | ¹H NMR (400 MHz, DMSO-d₆) δ 9.23-8.88 (m, 1H), 8.42-8.32 (m, 1H), 8.18-8.07 (m, 2H), 7.64-7.57 (m, 1H), 7.01-6.82 (m, 3H), 5.23-5.08 (m, 1H), 4.67-4.56 (m, 1H), 4.49-4.40 (m, 1H), 3.72-3.66 (m, 2H), 3.53-3.48 (m, 2H), 2.69 (s, 3H), 2.25- 1.77 (m, 9H). |
| 50 | ((3-((E)-4- (((5S,8S,10aR)- 3-acetyl- 8- (methyl(phenyl) carbamoyl)- 6- oxodecahydro- pyrrolo[1,2- a][1,5] diazocin-5- yl)amino)-4- oxobut-2-en- 2- yl)phenyl)diflu- oromethyl) phosphonic acid | 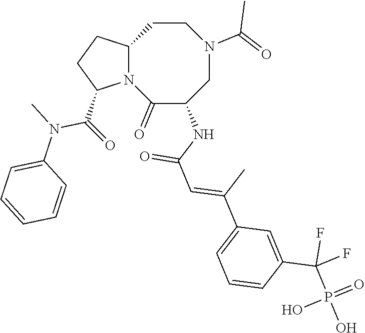 [SMILES: CC(=O)N1CC[C@H]2CC[C@@H](C(=O)N(C)c3ccccc3)N2C(=O)[C@@H](NC(=O)/C=C(\C)c2cccc(C(F)(F)P(=O)(O)O)c2)C1] | 633.3 [M + H]⁺ | ¹H NMR (400 MHz, DMSO-d₆) δ 8.39-8.20 (m, 1H), 7.72-7.33 (m, 9H), 6.61-6.38 (m, 1H), 4.80-4.67 (m, 1H), 4.21-4.17 (m, 1H), 4.12-4.08 (m, 1H), 3.83-3.76 (m, 2H), 3.34-3.27 (m, 1H), 3.21-3.05 (m, 4H), 2.49-2.45 (m, 3H), 2.24-2.08 (m, 3H), 2.04-1.60 (m, 6H). |
| 51 | ((3-((E)-3- (((5S,8S,10aR)- 3-acetyl- 8- (methyl(phenyl) carbamoyl)- 6- oxodecahydro- pyrrolo[1,2- a][1,5]diazocin- 5- yl)amino)-3- oxoprop-1- en-1- yl)phenyl)diflu- oromethyl) phosphonic acid | 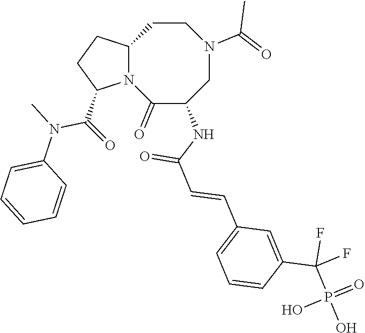 | 633.3 [M + H]⁺ | ¹H NMR (400 MHz, DMSO-d₆) δ 8.45-8.12 (m, 1H), 7.77-7.56 (m, 2H), 7.55-7.37 (m, 7H), 7.16-6.85 (m, 1H), 4.82-4.72 (m, 1H), 4.24-4.18 (m, 1H), 4.17-4.07 (m, 1H), 3.86-3.71 (m, 2H), 3.33-3.22 (m, 1H), 3.22-3.12 (m, 3H), 3.10-3.00 (m, 1H), 2.23-1.94 (m, 4H), 1.91-1.73 (m, 3H), 1.72-1.49 (m, 2H). |
| 52 | ((2- (((5S,8S,10aR)- 3-acetyl- 8-((3S,4R or 3R,4S)-3- cyano-4- phenylpyrroli- dine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 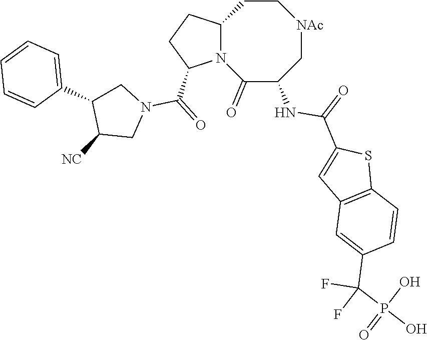 | 714.0 [M + H]⁺ | ¹H NMR (400 MHz, DMSO-d₆) δ 8.37-8.27 (m, 1H), 8.19-8.05 (m, 2H), 7.65-7.55 (m, 1H), 7.50-7.28 (m, 5H), 5.03-4.83 (m, 1H), 4.71-4.58 (m, 1H), 4.37-4.23 (m, 2H), 3.87-3.82 (m, 3H), 3.73-3.69 (m, 2H), 3.58-3.48 (m, 2H), 3.41-3.23 (m, 2H), 2.36-2.23 (m, 1H), 2.21-2.07 (m, 3H), 2.06-1.89 (m, 2H), 1.87-1.64 (m, 3H). |
| | | or | | |
| | | 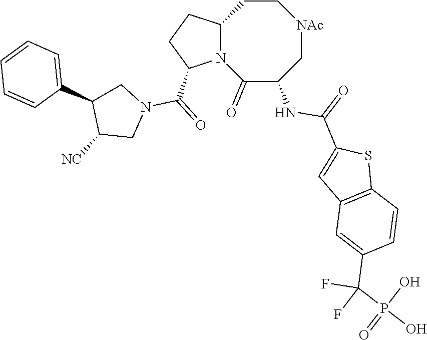 | | |
| | | SFC peak 2 was used for | | |
| | | biological testing | | |
| 53 | ((2- (((5S,8S,10aR)- 8-([1,1′- biphenyl]-4- yl(methyl)car- bamoyl)-3- acetyl-6- oxodecahydro- pyrrolo[1,2- a][1,5]diazocin- 5- yl)carbamoyl)- 1H-indol-5- yl)difluorometh- yl)phosphon- ic acid | 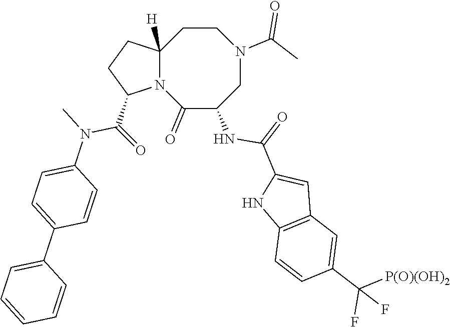 | 708 [M + H]⁺ | ¹H NMR (400 MHz, DMSO-d₆) δ 12.22- 11.65 (m, 1H), 8.93- 8.31 (m, 1H), 7.99-7.27 (m, 13H), 5.04-4.81 (m, 1H), 4.40-4.29 (m, 1H), 4.24-4.15 (m, 1H), 4.07-3.98 (m, 1H), 3.91-3.85 (m, 1H), 3.82-3.74 (m, 2H), 3.19 (s, 3H), 2.25- 2.08 (m, 3H), 1.98-1.62 (m, 6H). |
| 54 | ((2- (((5S,8S,10aR)- 3-acetyl- 8- (benzyl(meth- yl)carbamoyl)- 6- oxodecahydro- pyrrolo[1,2- a][1,5]diazocin- 5- yl)carbamoyl)- 1H-indol-5- yl)difluorometh- yl)phosphon- ic acid | 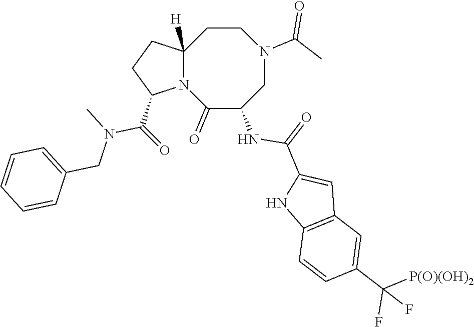 | 646.1 [M + H]⁺ | ¹H NMR (400 MHz, DMSO-d₆) δ 11.95- 11.62 (m, 1H), 8.89- 8.30 (m, 1H), 8.02-7.77 (m, 1H), 7.43-7.24 (m, 8H), 5.12-4.98 (m, 1H), 4.92-4.73 (m, 2H), 4.62-4.38 (m, 2H), 4.33-4.24 (m, 1H), 4.00-3.74 (m, 3H), 3.03-2.96 (m, 2H), 2.79-2.75 (m, 1H), 2.24-1.71 (m, 9H). |
| 55 | ((2- (((5S,8S,10aR)- 3-acetyl- 8- (diphenylcar- bamoyl)-6- oxodecahydro- pyrrolo[1,2- a][1,5]diazocin- 5- yl)carbamoyl)- 1H-indol-5- yl)difluorometh- yl)phosphon- ic acid | 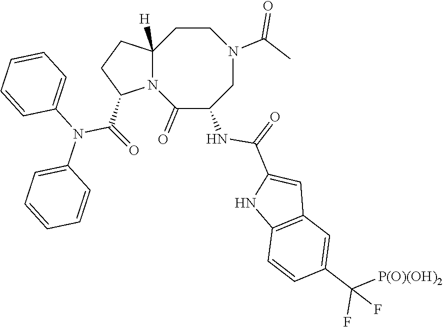 | 694 [M + H]⁺ | ¹H NMR (400 MHz, DMSO-d₆) δ 11.98- 11.72 (m, 1H), 8.83- 8.41 (m, 1H), 7.98-7.80 (m, 1H), 7.52-7.19 (m, 13H), 5.07-4.87 (m, 1H), 4.37-4.29 (m, 1H), 4.26-4.19 (m, 1H), 3.87-3.81 (m, 2H), 3.67-3.65 (m, 2H), 2.18-1.75 (m, 9H). |
| 56 | ((2- (((5S,8S,10aR)- 3-acetyl- 8- (benzyl(phenyl) carbamoyl)- 6- oxodecahydro- pyrrolo[1,2- a][1,5]diazocin- 5- yl)carbamoyl)- 1H-indol-5- yl)difluorometh- yl)phosphon- ic acid | 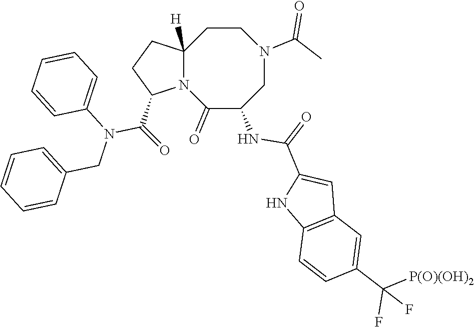 | 706.3 [M − H]⁻ | ¹H NMR (400 MHz, DMSO-d₆) δ 12.02- 11.74 (m, 1H), 8.87- 8.41 (m, 1H), 7.80 (s, 1H), 7.52-7.16 (m, 13H), 5.06-4.73 (m, 3H), 4.30-4.20 (m, 2H), 3.88-3.86 (m, 1H), 3.52-3.35 (m, 3H), 2.21-2.10 (m, 3H), 1.97-1.63 (m, 6H). |
| 57 | ((2- (((5S,8S,10aR)- 3-acetyl- 6-oxo-8- (1,2,3,4- tetrahydroquin- oline-1- carbonyl)deca- hydropyrrolo [1,2- a][1,5]diazocin- 5- yl)carbamoyl)- 1H-indol-5- yl)difluorometh- yl)phosphon- ic acid | 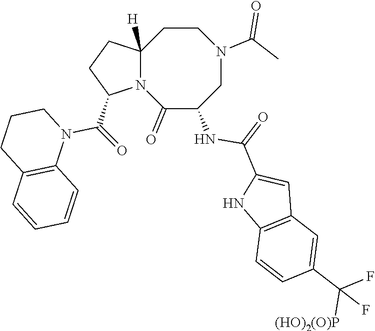 | 658.2 [M + H]⁺ | ¹H NMR (400 MHz, DMSO-d₆) δ 11.99- 11.68 (m, 1H), 8.81- 8.33 (m, 1H), 7.83 (s, 1H), 7.60-7.00 (m, 7H), 5.07-4.79 (m, 2H), 4.35-4.29 (m, 1H), 3.86-3.82 (m, 2H), 3.75-3.66 (m, 2H), 3.47-3.30 (m, 2H), 2.86-2.62 (m, 2H), 2.37-1.55 (m, 11H). |
| 58 | ((2- (((5S,8S,10aR)- 8-([1,1′- biphenyl]-4- ylcarbamoyl)- 3-acetyl-6- oxodecahydro- pyrrolo[1,2- a][1,5]diazocin- 5- yl)carbamoyl)- 1H-indol-5- yl)difluorometh- yl)phosphon- ic acid | 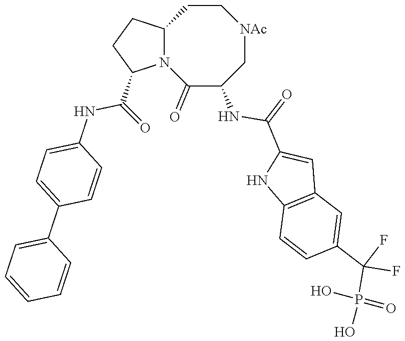 | 694.3 [M + H]⁺ | ¹H NMR (400 MHz, DMSO-d₆) δ 12.22- 11.49 (m, 1H), 10.48- 10.13 (m, 1H), 8.77- 8.34 (m, 1H), 7.97-7.79 (m, 1H), 7.77-7.68 (m, 2H), 7.67-7.59 (m, 4H), 7.46-7.39 (m, 3H), 7.36-7.28 (m, 2H), 5.13-4.91 (m, 1H), 4.60-4.49 (m, 1H), 4.41-4.30 (m, 1H), 3.91-3.84 (m, 1H), 3.78-3.73 (m, 1H), 3.65-3.59 (m, 1H), 3.38-3.33 (m, 1H), 2.34-2.07 (m, 5H), 2.03-1.89 (m, 2H), 1.87-1.69 (m, 2H). |
| 59 | ((2- (((5S,8S,10aR)- 3-acetyl- 8-((3R,4S or 3S,4R)-3- carbamoyl-4- phenylpyrroli- dine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 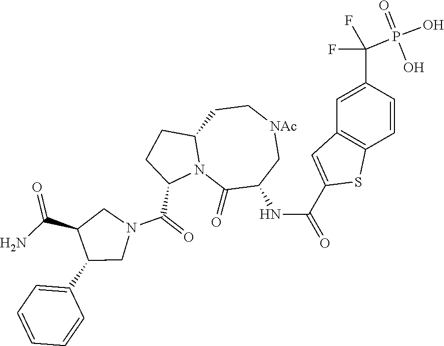 | 730.0 [M − H]⁻ | ¹H NMR (600 MHz, DMSO-d₆) δ 8.35-8.29 (m, 1H), 8.08 (q, J = 8.5 Hz, 3H), 7.59 (d, J = 8.5 Hz, 1H), 7.42-7.26 (m, 5H), 7.24 (ddt, J = 9.9, 7.2, 4.2 Hz, 1H), 7.01- 6.93 (m, 1H), 4.97 (d, J = 7.2 Hz, 1H), 4.65- 4.55 (m, 1H), 4.25 (s, 1H), 3.94 (dd, J = 10.9, 7.7 Hz, 1H), 3.90-3.69 (m, 4H), 3.65-3.59 (m, 1H), 3.53-3.44 (m, 2H), 3.06-2.99 (m, 1H), 2.25-2.14 (m, 3H), 2.12-2.07 (m, 1H), 1.94 (s, 2H), 1.86- 1.76 (m, 2H), 1.72 (dd, J = 11.8, 6.0 Hz, 2H). |
| | | or | | |
| | | 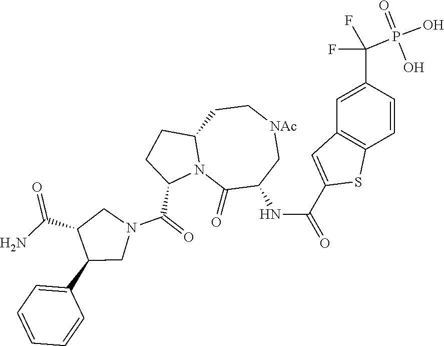 | | |
| | | SFC peak 1 was used for | | |
| | | biological testing | | |
| 60 | ((2- (((5S,8S,10aR)- 3-acetyl- 8-((rel- trans)-3- hydroxy-4- phenylpyrroli- dine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 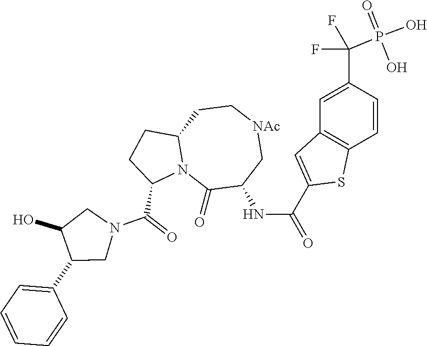 | 703.0 [M − H]⁻ | ¹H NMR (400 MHz, DMSO-d₆) δ 9.05-8.60 (m, 1H), 8.33 (d, J = 4.3 Hz, 1H), 8.14 (d, J = 8.9 Hz, 1H), 8.09 (s, 1H), 7.59 (d, J = 8.8 Hz, 1H), 7.37-7.21 (m, 5H), 4.91 (m, 2H), 2.71-4.48 (m, 2H), 4.31-4.20 (m, 2H), 4.00 (d, J = 9.6 Hz, 1H), 3.92-3.83 (m, 2H), 3.75 (d, J = 16.4 Hz, 2H), 3.62 (dd, J = 12.2, 6.2 Hz, 2H), 3.22-3.10 (m, 2H), 2.24-2.08 (m, 3H), 1.95 (s, 2H), 1.82 (s, 1H), 1.74 (d, J = 12.7 Hz, 1H). |
| 61 | ((2- (((5S,8S,10aR)- 3-acetyl- 8-(4′-cyano- 2- oxospiro[indo- line-3,3′- pyrrolidine]- 1′-carbonyl)- 6- oxodecahydro- pyrrolo[1,2- a][1,5]diazocin- 5- yl)carbamoyl) benzo[b]thio- phen-5- yl)difluorometh- yl)phosphon- ic acid | 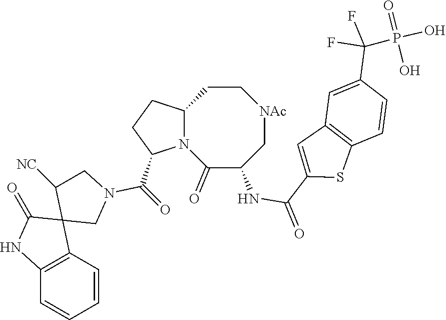 | 753.0 [M − H]⁻ | ¹H NMR (500 MHz, DMSO-d₆) δ 11.11- 10.81 (m, 2H), 9.15- 8.95 (m, 1H), 8.71 (m, 1H), 8.35-8.26 (m, 1H), 8.13-8.03 (m, 2H), 7.58 (m, 1H), 7.35- 7.22 (m, 2H), 7.08 (m, 1H), 6.95 (m, 1H), 4.99 (s, 1H), 4.84 (m, 1H), 4.73-4.63 (m, 1H), 4.57 (m, 1H), 4.28 (m, 2H), 4.13 (m, 2H), 4.00-3.91 (m, 2H), 3.83 (m, 2H), 2.17-2.06 (m, 3H), 2.00- 1.57 (m, 6H). |
| 62 | ((2- (((5S,8S,10aR)- 3-acetyl- 8-(4-benzyl- 3-cyano- piperazine- 1- carbonyl)-6- oxodecahydro- pyrrolo[1,2- a] [1,5]diazocin- 5- yl)carbamoyl) benzo[b]thio- phen-5- yl)difluorometh- yl)phosphon- ic acid | 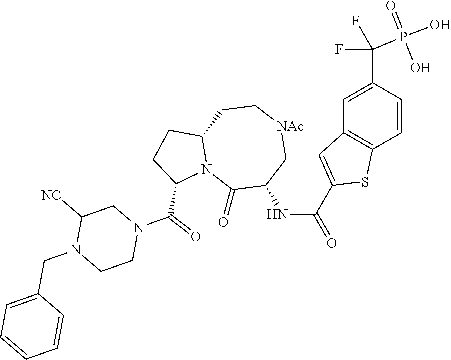 | 741.4 [M − H]⁻ | ¹H NMR (600 MHz, DMSO-d₆) δ 8.54 (m, 1H), 8.35 (m, 1H), 8.28 (m, 1H), 8.10 (d, J = 9.2 Hz, 1H), 8.07 (s, 1H), 7.58 (m, 1H), 7.40-7.18 (m, 4H), 4.96 (s, 1H), 4.88-4.76 (m, 1H), 4.57 (m, 1H), 4.38 (m, 1H), 4.27 (s, 1H), 4.06 (m, 2H), 3.86 (m, 1H), 3.78- 3.55 (m, 4H), 2.88 (s, 1H), 2.74 (m, 2H), 2.35 (m, 2H), 2.23-2.05 (m, 3H), 1.97 (m, 2H), 1.82 (s, 1H), 1.77-1.57 (m, 2H). |
| 63 | ((2- (((5S,8S,10aR)- 3-acetyl- 8-(3-cyano- 4- cyclopropylpi- perazine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 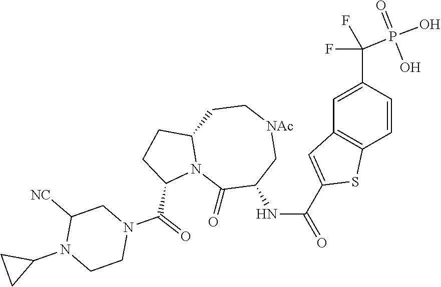 | 691.2 [M − H]⁻ | ¹H NMR (600 MHz, DMSO-d₆) δ 8.72-8.50 (m, 1H), 8.34-8.23 (m, 1H), 8.15-8.01 (m, 2H), 7.61-7.52 (m, 1H), 4.96 (s, 1H), 4.90- 4.77 (m, 1H), 4.52 (t, J = 14.5 Hz, 1H), 4.41- 4.22 (m, 2H), 4.22-4.13 (m, 1H), 3.86 (m, 1H), 3.68 (s, 2H), 3.03 (m, 2H), 2.91-2.74 (m, 2H), 2.21-2.02 (m, 3H), 1.92 (s, 1H), 1.81 (s, 2H), 1.76-1.53 (m, 2H), 1.37-1.17 (m, 1H), 1.13 (m, 2H), 0.53 (m, 2H), 0.47-0.25 (m, |
| | | | | 2H). |
| 64 | ((2- (((5S,8S,10aR)- 3-acetyl- 8-(8-cyano- 6- azaspiro[3.4] octane-6- carbonyl)-6- oxodecahydro- pyrrolo[1,2- a][1,5]diazocin- 5- yl)carbamoyl) benzo[b]thio- phen-5- yl)difluorometh- | 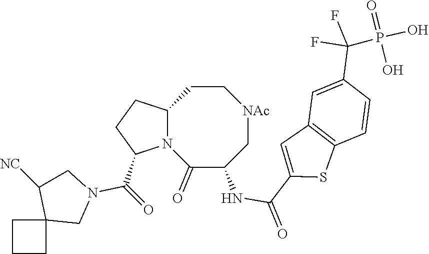 | 676.2 [M − H]⁻ | ¹H NMR (400 MHz, DMSO-d₆) δ 9.14-8.43 (m, 1H), 8.32 (d, J = 8.4 Hz, 1H), 8.13 (d, J = 7.1 Hz, 1H), 8.08 (s, 1H), 7.59 (d, J = 8.7 Hz, 1H), 4.91 (m, 1H), 4.57 (d, J = 9.9 Hz, 1H), 4.26 (s, 1H), 3.84 (d, J = 11.7 Hz, 3H), 3.72-3.61 (m, 2H), 3.58 (m, 2H), 3.41- 3.30 (m, 2H), 2.32 (s, 1H), 2.21-2.12 (m, 3H), 2.10-2.08 (m, 1H), 2.02 (s, 3H), 1.96- 1.87 (m, 4H), 1.74 (s, |
| | yl)phosphon- | | | 3H). |
| | ic acid | | | |
| 65 | ((2- (((5S,8S,10aR)- 3-acetyl- 6-oxo-8-((S)- 3-(pyridin-3- ylmethyl)pyr- rolidine-1- carbonyl)deca- hydropyrrolo [1,2- a][1,5]diazocin- 5- yl)carbamoyl) benzo[b]thio- phen-5- yl)difluorometh- yl)phosphon- | 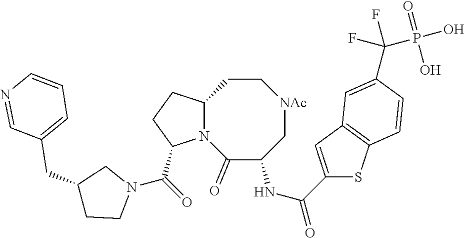 or | 702.2 [M − H]⁻ | |
| | ic acid | | | |
| | or | | | |
| | ((2- (((5S,8S,10aR)- 3-acetyl- 6-oxo-8- ((R)-3- (pyridin-3- ylmethyl)pyr- rolidine-1- carbonyl)deca- hydropyrrolo [1,2- a][1,5]diazocin- 5- yl)carbamoyl) benzo[b]thio- | 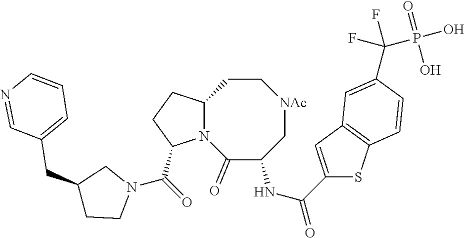 | | |
| | phen-5- | | | |
| | yl)difluorometh- | | | |
| | yl)phosphon- | | | |
| | ic acid | | | |
| 66 | ((2- (((5S,8S,10aR)- 3-acetyl- 6-oxo-8-((S)- 3-(pyridin-4- ylmethyl)pyr- rolidine-1- carbonyl)deca- hydropyrrolo [1,2- a][1,5]diazocin- 5- yl)carbamoyl) benzo[b]thio- phen-5- yl)difluorometh- yl)phosphon- | 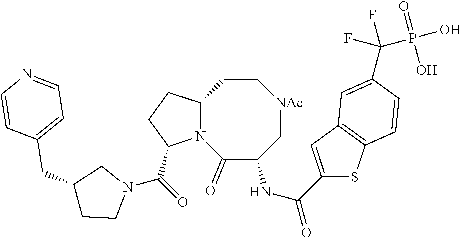 Or | 702.2 [M − H]⁻ | |
| | ic acid | | | |
| | or | | | |
| | ((2- (((5S,8S,10aR)- 3-acetyl- 6-oxo-8- ((R)-3- (pyridin-4- ylmethyl)pyr- rolidine-1- carbonyl)deca- hydropyrrolo [1,2- a][1,5]diazocin- 5- yl)carbamoyl) benzo[b]thio- | 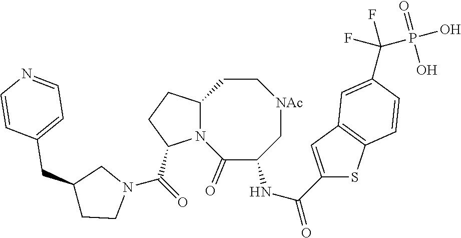 | | |
| | phen-5- | | | |
| | yl)difluorometh- | | | |
| | yl)phosphon- | | | |
| | ic acid | | | |
| 67 | ((2- (((5S,8S,10aR)- 3-acetyl- 8-((7-methyl- 1H-indazol- 5- yl)carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 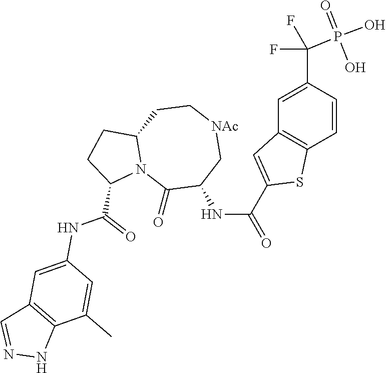 | 687.2 [M − H]⁻ | ¹H NMR (600 MHz, DMSO-d₆) δ 9.92 (m, 1H), 8.98 (d, J = 7.5 Hz, 1H), 8.62 (d, J = 7.0 Hz, 1H), 8.28 (s, 1H), 8.24- 8.07 (m, 2H), 8.07-8.04 (m, 1H), 7.57 (m, 1H), 7.36 (m, 1H), 6.37 (dd, J = 12.6, 9.6 Hz, 1H), 5.01-4.82 (m, 1H), 4.42 (d, J = 8.6 Hz, 1H), 4.33- 4.25 (m, 1H), 3.94 (d, J = 13.2 Hz, 1H), 3.84 (dd, J = 14.4, 3.7 Hz, 1H), 3.72 (m, 2H), 3.40 (s, 3H), 2.21 (m, 1H), 2.11 (m, 3H), 2.07-2.01 (m, 1H), 1.97-1.87 (m, 2H), 1.87-1.76 (m, 1H), 1.74 (m, 1H). |
| 68 | ((2- (((5S,8S,10aR)- 3-acetyl- 8-((3- methylbenzo [d]isoxazol- 5- yl)carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 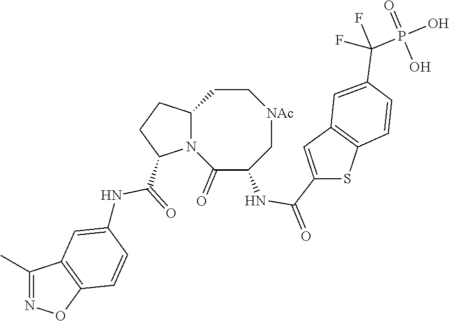 | 688 [M − H]⁻ | ¹H NMR (400 MHz, DMSO-d₆) δ 10.40 (d, J = 16.0 Hz, 1H), 8.84 (dd, J = 139.3, 7.2 Hz, 1H), 8.29 (s, 1H), 8.19 (d, J = 23.8 Hz, 1H), 8.13 (dd, J = 8.9, 2.8 Hz, 1H), 8.07 (s, 1H), 7.62 (dq, J = 15.1, 8.8 Hz, 3H), 4.93 (d, J = 69.5 Hz, 2H), 4.52 (q, J = 8.2 Hz, 1H), 4.32 (s, 2H), 4.00 (d, J = 12.7 Hz, 1H), 3.88-3.82 (m, 2H), 3.39 (d, J = 12.8 Hz, 3H), 2.28 (s, 1H), 2.14 (d, J = 16.7 Hz, 3H), 2.08 (d, J = 8.5 Hz, 1H), 1.95 (d, J = |
| | | | | 10.8 Hz, 2H), 1.87- |
| | | | | 1.71 (m, 2H). |
| 69 | ((2- (((5S,8S,10aR)- 3-aceytl- 6-oxo-8- (quinoxalin- 6- ylcarbamoyl) decahydropyr- rolo[1,2- a][1,5]diazocin- 5- yl)carbamoyl) benzo[b]thio- phen-5- yl)difluorometh- yl)phosphon- ic acid | 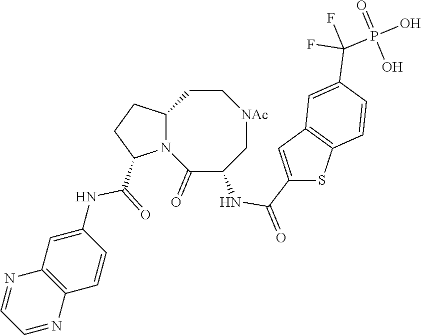 | 685.2 [M − H]⁻ | ¹H NMR (600 MHz, DMSO-d₆) δ 10.67 (m, 1H), 8.86 (m, 1H), 8.80 (m, 1H), 8.49 (m, 1H), 8.28 (m, 1H), 8.23-8.15 (m, 1H), 8.13-8.09 (m, 1H), 8.05 (m, 1H), 8.02 (m, 1H), 7.91 (m, 1H), 7.57 (d, J = 8.7 Hz, 1H), 5.10-4.88 (m, 1H), 4.56 (m, 1H), 4.34 (m, 1H), 3.80 (m, 1H), 3.68 (s, 1H), 3.48 (s, 2H), 2.30 (m, 1H), 2.14 (s, 3H), 2.07-1.97 (m, 2H), 1.86 (m, 2H), 1.78 (m, 1H). |
| 70 | ((2- (((5S,8S,10aR)- 3-acetyl- 8-((4- (methylsulfo- nyl)phenyl)car- bamoyl)-6- oxodecahydro- pyrrolo[1,2- a][1,5]diazocin- 5- yl)carbamoyl) benzo[b]thio- phen-5- yl)difluorometh- yl)phosphon- ic acid | 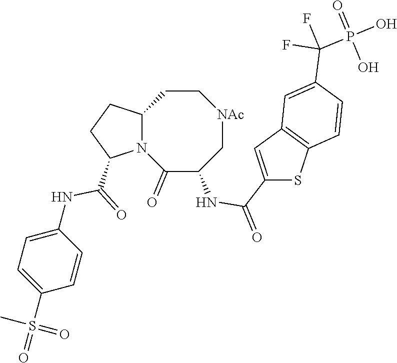 | 711.0 [M − H]⁻ | ¹H NMR (600 MHz, DMSO-d₆) δ 10.61 (m, 1H), 9.00 (m, 1H), 8.78- 8.59 (m, 1H), 8.27 (m, 1H), 8.15-7.97 (m, 2H), 7.88-7.75 (m, 3H), 7.57 (m, 1H), 5.06- 4.86 (m, 1H), 4.50 (m, 1H), 4.31 (m, 1H), 3.88 (m, 1H), 3.84-3.74 (m, 1H), 3.47-3.40 (m, 2H), 3.13 (s, 3H), 3.03 (m, 1H), 2.28 (m, 1H), 2.15-2.04 (s, 3H), 2.03- 1.86 (m, 2H), 1.87- 1.69 (m, 2H). |
| 71 | ((2- (((5S,8S,10aR)- 3-acetyl- 8-((1-methyl- 6-oxo-1,6- dihydropyridin- 3- yl)carbamoyl)- 6- oxodecahydro- pyrrolo[1,2- a] [1,5]diazocin- 5- yl)carbamoyl) benzo[b]thio- phen-5- yl)difluorometh- yl)phosphon- ic acid | 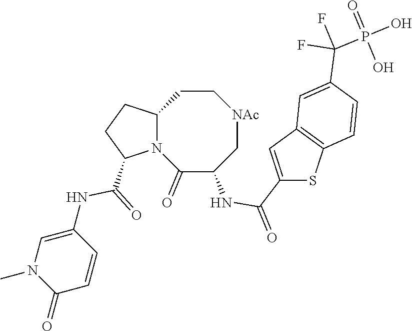 | 664.2 [M − H]⁻ | ¹H NMR (600 MHz, DMSO-d₆) δ 9.92 (m 1H), 8.62 (m, 1H), 8.28 (s, 1H), 8.16-8.00 (m, 3H), 7.57 (m, 1H), 7.36 (m, 1H), 6.37 (m, 1H), 5.01-4.82 (m, 1H), 4.42 (m, 1H), 4.33-4.24 (m, 1H), 3.94 (m, 1H), 3.84 (m, 1H), 3.72 (m, 2H), 3.40 (s, 3H), 2.21 (m, 1H), 2.11 (s, 3H), 2.08- 1.99 (m, 1H), 1.98-1.85 (m, 2H), 1.74 (m, 2H). |
| 72 | ((2- (((5S,8S,10aR)- 3-acetyl- 8- (imidazo[1,2- a]pyridin-6- ylcarbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 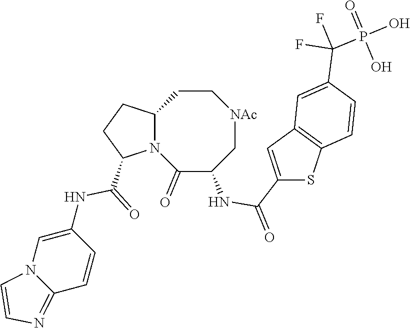 | 674.0 [M − H]⁻ | ¹H NMR (500 MHz, DMSO-d₆) δ 10.56 (m, 1H), 9.32 (m, 1H), 8.70 (m, 1H), 8.24-8.10 (m, 2H), 8.03-7.88 (m, 2H), 7.81 (s, 1H), 7.63 (m, 1H), 7.55 (m, 1H), 7.43 (m, 1H), 5.00 (s, 1H), 4.54-4.47 (m, 1H), 4.31 (s, 1H), 3.97 (s, 1H), 3.87 (m, 1H), 3.74 (m, 2H), 3.36 (m, 2H), 2.33-2.04 (s, 3H), 2.10-1.71 (m, 4H). |
| 73 | ((2- (((5S,8S,10aR)- 3-acetyl- 8-((4-(3- (dimethylami- no)propoxy) phenyl)carba- moyl)-6- oxodecahydro- pyrrolo[1,2- a][1,5]diazocin- 5- yl)carbamoyl) benzo[b]thio- phen-5- yl)difluorometh- yl)phosphon- ic acid | 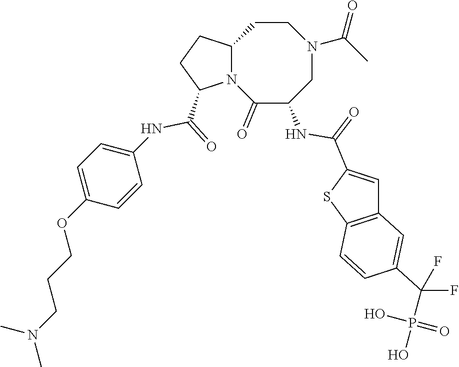 [SMILES: CC(=O)N1CC[C@H]2CC[C@@H](C(=O)Nc3ccc(OCCCN(C)C)cc3)N2C(=O)[C@@H](NC(=O)c2cc3cc(C(F)(F)P(=O)(O)O)ccc3s2)C1] | 734.4 [M − H]⁻ | ¹H NMR (600 MHz, DMSO-d₆) δ 11.12 (s, 2H), 10.05 (m, 1H), 8.63 (m, 1H), 8.26 (m, 1H), 8.03 (m, 1H), 7.99 (m, 1H), 7.61 (m, 1H), 7.48 (m, 2H), 6.81 (m, 2H), 4.92 (m, 1H), 4.51-4.42 (m, 1H), 4.25 (m, 1H), 3.89 (m, 3H), 3.82 (m, 1H), 3.70 (m, 2H), 3.56 (m, 2H), 3.03 (m, 2H), 2.63 (s, 6H), 2.22 (s, 1H), 2.16-2.05 (s, 3H), 2.06-1.83 (m, 5H), 1.73 (m, 2H). |
| 74 | ((2- (((5S,8S,10aR)- 3-acetyl- 8-((4-(2- methoxyethoxy) phenyl)car- bamoyl)-6- oxodecahydro- pyrrolo[1,2- a][1,5]diazocin- 5- yl)carbamoyl) benzo[b]thio- phen-5- yl)difluorometh- yl)phosphon- ic acid | 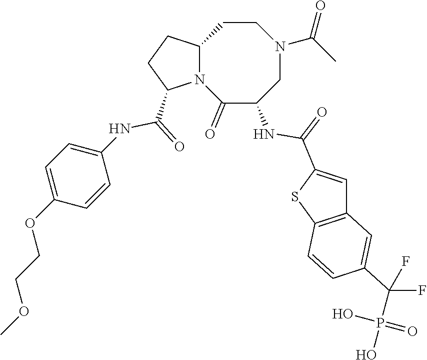 [SMILES: COCCOc1ccc(NC(=O)[C@@H]2CC[C@@H]3CCN(C(C)=O)C[C@H](NC(=O)c4cc5cc(C(F)(F)P(=O)(O)O)ccc5s4)C(=O)N32)cc1] | 707.2 [M − H]⁻ | |
| 75 | ((2- (((5S,8S,10aR)- 3-acetyl- 8-((4-(2- (dimethylami- no)ethoxy)phe- nyl)carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 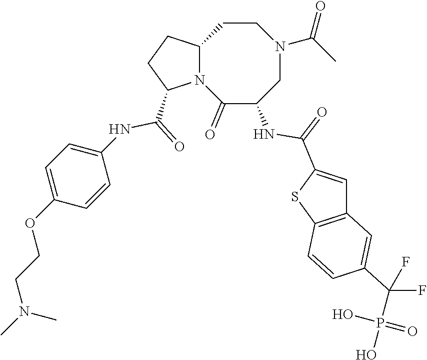 [SMILES: CC(=O)N1CC[C@H]2CC[C@@H](C(=O)Nc3ccc(OCCN(C)C)cc3)N2C(=O)[C@@H](NC(=O)c2cc3cc(C(F)(F)P(=O)(O)O)ccc3s2)C1] | 720.2 [M − H]⁻ | ¹H NMR (600 MHz, DMSO-d₆) δ 10.84 (s, 2H), 10.03 (m, 1H), 8.63 (m, 1H), 8.21 (m, 1H), 8.07-7.94 (m, 2H), 7.60 (m, 1H), 7.49 (m, 2H), 6.88 (m, 2H), 5.00 (s, 1H), 4.44 (m, 1H), 4.30 (m, 1H), 4.21 (m, 2H), 3.88 (s, 1H), 3.82 (m, 1H), 3.67 (m, 2H), 3.36 (s, 2H), 2.73 (s, 6H), 2.23 (s, 1H), 2.11 (s, 3H), 2.07-2.00 (m, 1H), 1.89 (m, 2H), 1.84- 1.69 (m, 2H). |
| 76 | ((2- (((5S,8S,10aR)- 3-acetyl- 6-oxo-8-(2- oxospiro[indo- line-3,3′- pyrrolidine]- 1′- carbonyl)deca- hydropyrrolo [1,2- a][1,5]diazocin- 5- yl)carbamoyl) benzo[b]thio- phen-5- yl)difluorometh- yl)phosphon- ic acid | 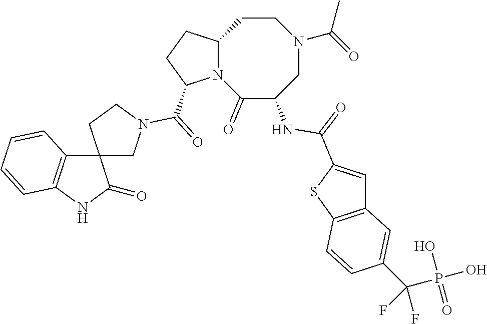 [SMILES: CC(=O)N1CC[C@H]2CC[C@@H](C(=O)N3CCC4(C3)C(=O)Nc3ccccc34)N2C(=O)[C@@H](NC(=O)c2cc3cc(C(F)(F)P(=O)(O)O)ccc3s2)C1] | 728.0 [M − H]⁻ | ¹H NMR (600 MHz, DMSO-d₆) δ 10.73- 10.35 (m, 1H), 9.33- 8.50 (m, 1H), 8.36-8.27 (m, 1H), 8.12 (m, 1H), 8.08 (m, 1H), 7.58 (m, 1H), 7.25-7.13 (m, 2H), 6.99 (m, 1H), 6.89- 6.83 (m, 1H), 5.04- 4.80 (m, 1H), 4.76-4.51 (m, 1H), 4.27 (m, 1H), 4.17-3.97 (m, 1H), 3.85 (m, 2H), 3.73 (m, 2H), 3.56-3.51 (m, 1H), 2.32 (m, 2H), 2.21-2.14 (s, 3H), 2.12-2.05 (m, 2H), 1.94 (s, 2H), 1.72 (m, 4H). |
| 77 | ((2- (((5S,8S,10aR)- 3-acetyl- 8-(3- (morpholine- 4- carbonyl)azeti- dine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 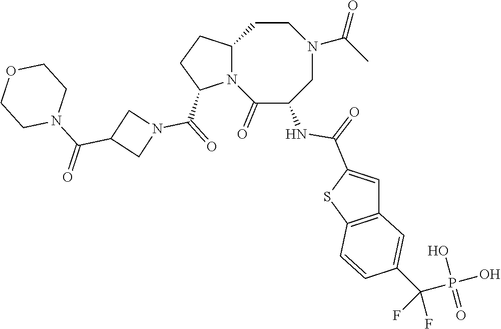 [SMILES: CC(=O)N1CC[C@H]2CC[C@@H](C(=O)N3CC(C(=O)N4CCOCC4)C3)N2C(=O)[C@@H](NC(=O)c2cc3cc(C(F)(F)P(=O)(O)O)ccc3s2)C1] | 710.2 [M − H]⁻ | ¹H NMR (400 MHz, DMSO-d₆) δ 9.23-8.63 (m, 1H), 8.30 (m, 1H), 8.05 (m, 2H), 7.67-7.52 (m, 1H), 4.92 (m, 1H), 4.71-4.42 (m, 1H), 4.38- 4.17 (m, 3H), 4.12- 3.87 (m, 3H), 3.80 (s, 2H), 3.38-3.32 (m, 2H), 3.26 (m, 2H), 2.98 (m, 6H), 2.17 (s, 1H), 2.10 (s, 3H), 2.00 (s, 1H), 1.74 (m, 4H). |
| 78 | ((2- (((5S,8S,10aR)- 3-acetyl- 8-(3-(1,1- dioxidoisothi- azolidin-2- yl)pyrrolidine- 1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 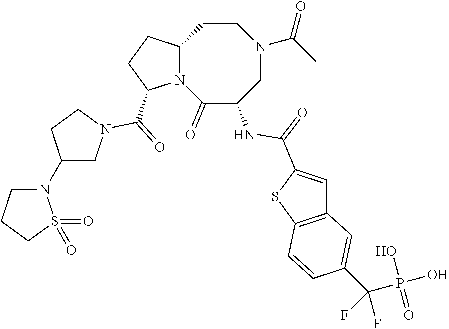 [SMILES: CC(=O)N1CC[C@H]2CC[C@@H](C(=O)N3CCC(N4CCCS4(=O)=O)C3)N2C(=O)[C@@H](NC(=O)c2cc3cc(C(F)(F)P(=O)(O)O)ccc3s2)C1] | 732.2 [M + H]⁺ | |
| 79 | ((2- (((5S,8S,10aR)- 3-acetyl- 8- ((3aR,3bS,6aR, 6bS)-2,2- dioxidooctahy- dro-1H- thieno[3′,4′:3,4] cyclobuta[1,2- c]pyrrole-5- carbonyl)-6- oxodecahydro- pyrrolo[1,2- a][1,5]diazocin- 5- yl)carbamoyl) benzo[b]thio- phen-5- yl)difluorometh- | 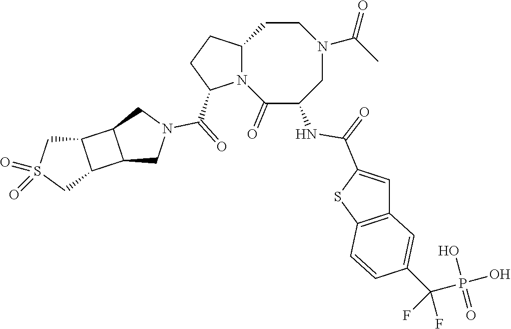 [SMILES: CC(=O)N1CC[C@H]2CC[C@@H](C(=O)N3C[C@@H]4[C@H](C3)[C@H]3CS(=O)(=O)C[C@@H]43)N2C(=O)[C@@H](NC(=O)c2cc3cc(C(F)(F)P(=O)(O)O)ccc3s2)C1] | 727.8 [M − H]⁻ | ¹H NMR (400 MHz, DMSO-d₆) δ 9.13-8.51 (m, 2H), 8.29 (s, 1H), 8.07 (m, 2H), 7.59 (m, 1H), 4.99 (s, 1H), 4.81 (m, 1H), 4.64 (s, 1H), 4.28 (s, 2H), 3.96 (m, 2H), 3.88 (s, 2H), 3.81 (s, 6H), 3.11-3.05 (m, 1H), 2.91 (s, 1H), 2.80 (s, 1H), 2.17 (s, 2H), 2.07 (s, 3H), 1.98-1.53 (m, 5H). |
| | yl)phosphon- | | | |
| | ic acid | | | |
| 80 | ((2- (((5S,8S,10aR)- 3-acetyl- 8-(5,5- dioxidooctahy- drothiopyrano [3,4- c]pyrrole-2- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 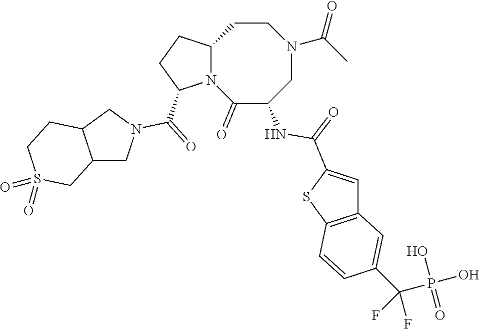 [SMILES: CC(=O)N1CC[C@H]2CC[C@@H](C(=O)N3CC4CCS(=O)(=O)CC4C3)N2C(=O)[C@@H](NC(=O)c2cc3cc(C(F)(F)P(=O)(O)O)ccc3s2)C1] | 715.0 [M − H]⁻ | ¹H NMR (400 MHz, DMSO-d₆) δ 8.77 (m, 1H), 8.30 (m, 1H), 8.08 (m, 2H), 7.59 (m, 1H), 4.90 (m, 1H), 4.61-4.45 (m, 1H), 4.24 (s, 2H), 3.89 (s, 9H), 3.13 (s, 2H), 2.89-2.78 (m, 1H), 2.25 (s, 1H), 2.15 (s, 3H), 2.07 (m, 2H), 2.01-1.67 (m, 6H). |
| 81 | ((2- (((5S,8S,10aR)- 3-acetyl- 6-oxo-8-(3- (5,6,7,8- tetrahydroimi- dazo[1,2- a]pyridin-3- yl)pyrrolidine- 1- carbonyl)deca- hydropyrrolo [1,2- a][1,5]diazocin- 5- yl)carbamoyl) benzo[b]thio- phen-5- yl)difluorometh- yl)phosphon- | 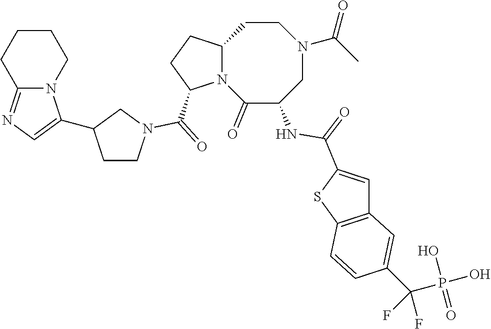 [SMILES: CC(=O)N1CC[C@H]2CC[C@@H](C(=O)N3CCC(c4cnc5n4CCCC5)C3)N2C(=O)[C@@H](NC(=O)c2cc3cc(C(F)(F)P(=O)(O)O)ccc3s2)C1] | 731.0 [M − H]⁻ | ¹H NMR (600 MHz, DMSO-d₆) δ 9.05 (m, 1H), 8.75-8.33 (m, 1H), 8.19 (m, 1H), 7.96 (m, 1H), 7.65-7.53 (m, 1H), 7.45-6.98 (m, 1H), 5.01 (m, 1H), 4.50 (s, 1H), 4.24 (s, 1H), 4.09-3.78 (m, 4H), 3.69 (s, 1H), 3.59 (s, 1H), 3.52 (s, 4H), 2.97 (s, 1H), 2.77 (s, 3H), 2.22 (s, 1H), 2.17-2.03 (m, 3H), 2.00 (s, 1H), 1.93 (m, 3H), 1.76 (m, 4H), 1.73-1.14 (m, 2H). |
| | ic acid | | | |
| 82 | ((2- (((5S,8S,10aR)- 3-acetyl- 6-oxo-8-(3- (thiazol-5- ylmethoxy)pyr- rolidine-1- carbonyl)deca- hydropyrrolo [1,2- a][1,5]diazocin- 5- yl)carbamoyl) benzo[b]thio- phen-5- yl)difluorometh- yl)phosphon- ic acid | 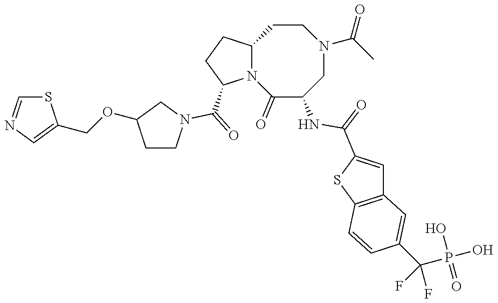 [SMILES: CC(=O)N1CC[C@H]2CC[C@@H](C(=O)N3CCC(OCc4cncs4)C3)N2C(=O)[C@@H](NC(=O)c2cc3cc(C(F)(F)P(=O)(O)O)ccc3s2)C1] | 724.0 [M − H]⁻ | ¹H NMR (600 MHz, DMSO-d₆) δ 9.11-9.03 (m, 1H), 8.57 (m, 1H), 8.33-8.25 (m, 1H), 8.14- 8.09 (m, 1H), 8.06 (m, 1H), 7.85 (m, 1H), 7.57 (m, 1H), 4.95 (s, 1H), 4.76 (m, 2H), 4.64-4.49 (m, 1H), 4.30-4.14 (m, 2H), 3.87 (m, 1H), 3.73 m, 1H), 3.65-3.53 (m, 2H), 3.51-3.39 (m, 3H), 2.26 (m, 2H), 2.23- 2.09 (m, 3H), 2.07 (m, 1H), 2.04-1.85 (m, 4H), 1.84-1.63 (m, 4H). |
| 83 | ((2- (((5S,8S,10aR)- 3-acetyl- 8-(3-(4- methoxypiper- idin-1- yl)pyrrolidine- 1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 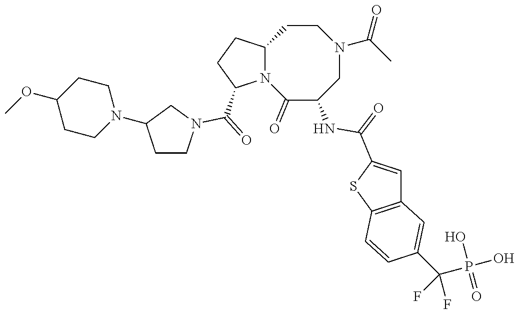 [SMILES: COC1CCN(C2CCN(C(=O)[C@@H]3CC[C@@H]4CCN(C(C)=O)C[C@H](NC(=O)c5cc6cc(C(F)(F)P(=O)(O)O)ccc6s5)C(=O)N43)C2)CC1] | 724.0 [M − H]⁻ | ¹H NMR (600 MHz, DMSO-d₆) δ 8.74 (m, 1H), 8.67-8.48 (m, 1H), 8.25 (m, 1H), 8.04- 7.96 (m, 1H), 7.60 (s, 1H), 4.89 (m, 1H), 4.63- 4.47 (m, 1H), 4.24 (s, 1H), 3.85 (m, 2H), 3.63 (m, 2H), 3.43 (s, 6H), 3.24-3.15 (m, 3H), 2.97 (s, 4H), 2.52 (s, 2H), 2.19-2.04 (m, 3H), 1.96 (m, 3H), 1.83-1.61 (m, 3H), 1.53 (s, 2H). |
| 84 | ((2- (((5S,8S,10aR)- 8-(3-(3- acetamido- 1H-pyrazol- 1- yl)pyrrolidine- 1- carbonyl)-3- acetyl-6- oxodecahydro- pyrrolo[1,2- a][1,5]diazocin- 5- yl)carbamoyl) benzo[b]thio- phen-5- yl)difluorometh- | 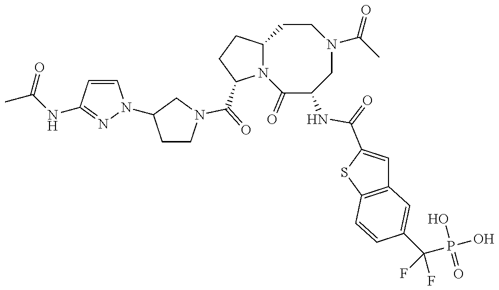 [SMILES: CC(=O)Nc1ccn(C2CCN(C(=O)[C@@H]3CC[C@@H]4CCN(C(C)=O)C[C@H](NC(=O)c5cc6cc(C(F)(F)P(=O)(O)O)ccc6s5)C(=O)N43)C2)n1] | 734.0 [M − H]⁻ | ¹H NMR (500 MHz, DMSO-d₆) δ 10.39 (m, 1H), 8.80 (m, 1H), 8.30 (m, 1H), 8.21-8.12 (m, 1H), 8.10 (m, 1H), 7.73- 7.62 (m, 1H), 7.58 (m, 1H), 6.50-6.39 (m, 1H), 4.96 (s, 1H), 4.88- 4.78 (m, 1H), 4.58 (m, 1H), 4.19 (m, 2H), 3.94 (s, 1H), 3.85 (m, 2H), 3.72 (s, 2H), 2.25 (m, 2H), 2.15 (m, 3H), 2.08 (m, 1H), 2.06-1.88 (m, 6H), 1.87-1.62 (m, 4H). |
| | yl)phosphon- | | | |
| | ic acid | | | |
| 85 | ((2- (((5S,8S,10aR)- 3-acetyl- 8-(3-(N-((5- cyclopropyl- 1,2,4- oxadiazol-3- yl)methyl)ace- tamido)pyrro- lidine-1- carbonyl)-6- oxodecahydro- pyrrolo[1,2- a][1,5]diazocin- 5- yl)carbamoyl) benzo[b]thio- phen-5- yl)difluorometh- yl)phosphon- | 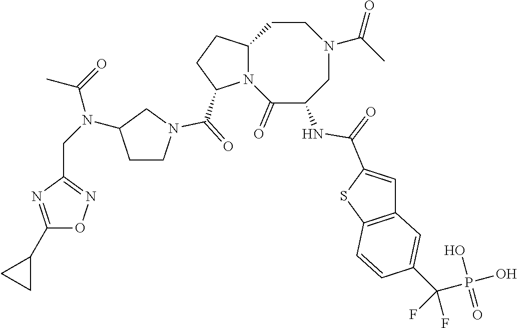 [SMILES: CC(=O)N1CC[C@H]2CC[C@@H](C(=O)N3CCC(N(Cc4noc(C5CC5)n4)C(C)=O)C3)N2C(=O)[C@@H](NC(=O)c2cc3cc(C(F)(F)P(=O)(O)O)ccc3s2)C1] | 790.2 [M − H]⁻ | ¹H NMR (500 MHz, DMSO-d₆) δ 8.97-8.52 (m, 1H), 8.34-8.24 (m, 1H), 8.17-8.09 (m, 1H), 8.07 (m, 1H), 7.57 (m, 1H), 4.99-4.79 (m, 1H), 4.68 (m, 2H), 4.50 (m, 2H), 4.23 (s, 2H), 3.94 (s, 1H), 3.54 (m, 2H), 3.27 (m, 2H), 3.22- 3.14 (m, 1H), 3.06 (m, 1H), 2.32-2.18 (m, 2H), 2.18-2.13 (m, 3H), 2.12-1.99 (m, 5H), 1.91 (m, 2H), 1.83- 1.65 (m, 3H), 1.24- 1.02 (m, 4H). |
| | ic acid | | | |
| 86 | ((2- (((5S,8S,10aR)- 3-acetyl- 6-oxo-8-(1- phenyl- 1,4,5,6- tetrahydropyr- rolo[3,4- c]pyrazole-5- carbonyl)deca- hydropyrrolo [1,2- a][1,5]diazocin- 5- yl)carbamoyl) benzo[b]thio- phen-5- yl)difluorometh- yl)phosphon- ic acid | 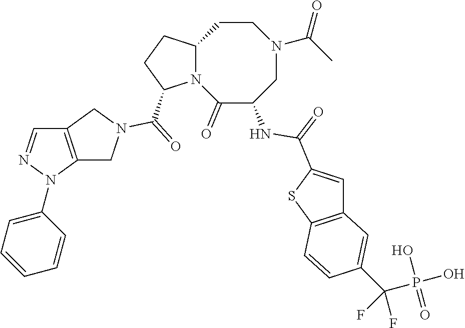 [SMILES: CC(=O)N1CC[C@H]2CC[C@@H](C(=O)N3Cc4cnn(-c5ccccc5)c4C3)N2C(=O)[C@@H](NC(=O)c2cc3cc(C(F)(F)P(=O)(O)O)ccc3s2)C1] | 725.0 [M − H]⁻ | ¹H NMR (600 MHz, DMSO-d₆) δ 8.64 (m, 1H), 8.28 (m, 1H), 8.11 (m, 1H), 8.05 (m, 1H), 7.71-7.55 (m, 4H), 7.48 m, 2H), 7.30 (m, 1H), 5.35-5.17 (m, 1H), 5.02 (s, 1H), 4.90 (m, 1H), 4.73 (m, 1H), 4.70-4.64 (m, 1H), 4.49-4.37 (m, 1H), 4.35-4.27 (m, 1H), 3.88-3.81 (m, 1H), 3.70 (m, 1H), 2.45- 2.38 (m, 2H), 2.18- 2.10 (s, 3H), 2.05 (m, 2H), 1.95-1.75 (m, 4H). |
| 87 | ((2- (((5S,8S,10aR)- 3-acetyl- 6-oxo-8-(1- (pyrimidin-2- yl)-1,4,5,6- tetrahydropyr- rolo[3,4- c]pyrazole-5- carbonyl)deca- hydropyrrolo [1,2- a][1,5]diazocin- 5- yl)carbamoyl) benzo[b]thio- phen-5- yl)difluorometh- yl)phosphon- ic acid | 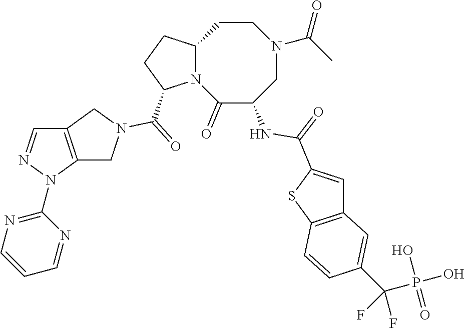 [SMILES: CC(=O)N1CC[C@H]2CC[C@@H](C(=O)N3Cc4cnn(-c5ncccn5)c4C3)N2C(=O)[C@@H](NC(=O)c2cc3cc(C(F)(F)P(=O)(O)O)ccc3s2)C1] | 727.0 [M − H]⁻ | ¹H NMR (600 MHz, DMSO-d₆) δ 9.00-8.64 (m, 3H), 8.31-8.23 (m, 1H), 8.10 (m, 1H), 8.03 (m, 1H), 7.72 (m, 1H), 7.55 (m, 1H), 7.47-7.37 (m, 1H), 5.27 (m, 1H), 5.13-4.98 (m, 1H), 4.94 (m, 1H), 4.84-4.73 (m, 1H), 4.73-4.46 (m, 2H), 4.45-4.21 (m, 2H), 3.82 (m, 1H), 2.12 (m, 3H), 2.06 (m, 1H), 1.90 (m, 2H), 1.87-1.60 (m, 3H). |
| 88 | ((2- (((5S,8S,10aR)- 3-acetyl- 8-(3-cyano- 3-(pyridin-2- yl)azetidine- 1-carbonyl)- 6- oxodecahydro- pyrrolo[1,2- a][1,5]diazocin- 5- yl)carbamoyl) benzo[b]thio- phen-5- yl)difluorometh- yl)phosphon- ic acid | 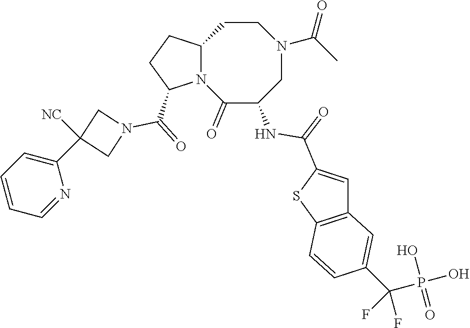 [SMILES: CC(=O)N1CC[C@H]2CC[C@@H](C(=O)N3CC(C#N)(c4ccccn4)C3)N2C(=O)[C@@H](NC(=O)c2cc3cc(C(F)(F)P(=O)(O)O)ccc3s2)C1] | 699.0 [M − H]⁻ | ¹H NMR (500 MHz, DMSO-d₆) δ 8.86-8.59 (m, 2H), 8.30 (m, 1H), 8.14-8.02 (m, 2H), 7.95 (m, 1H), 7.70 (m, 1H), 7.58 (s, 1H), 7.46 (m, 1H), 5.06 (m, 1H), 4.90 (m, 1H), 4.79-4.65 (m, 1H), 4.47 (m, 1H), 4.41- 4.32 (m, 2H), 4.27 (s, 1H), 3.76 (s, 1H), 3.65 (s, 1H), 3.47 (s, 1H), 3.07-2.99 (m, 1H), 2.08 (m, 3H), 2.01 (s, 2H), 1.78 (m, 4H). |
| 89 | ((2- (((5S,8S,10aR)- 3-acetyl- 8-((2- cyanopropyl) (methyl)carba- moyl)-6- oxodecahydro- pyrrolo[1,2- a][1,5]diazocin- 5- yl)carbamoyl) benzo[b]thio- phen-5- yl)difluorometh- yl)phosphon- ic acid | 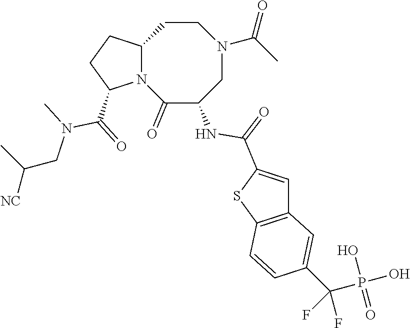 [SMILES: CC(=O)N1CC[C@H]2CC[C@@H](C(=O)N(C)CC(C)C#N)N2C(=O)[C@@H](NC(=O)c2cc3cc(C(F)(F)P(=O)(O)O)ccc3s2)C1] | 638.2 [M − H]⁻ | ¹H NMR (500 MHz, DMSO-d₆) δ 9.10-8.50 (m, 1H), 8.30 (m, 1H), 8.13-8.03 (m, 2H), 7.58 (m, 1H), 4.99 (s, 1H), 4.78 (m, 1H), 4.26 (s, 1H), 3.83 (m, 1H), 3.77- 3.62 (m, 3H), 3.59 (m, 2H), 3.14 (m, 3H), 3.08- 3.03 (m, 1H), 2.88 (m, 1H), 2.18-2.08 (m, 3H), 2.07-1.97 (m, 1H), 1.89 (s, 1H), 1.74 (m, 3H), 1.21-1.12 (m, 3H). |
| 90 | ((2- (((5S,8S,10aR)- 3-acetyl- 8-((2- cyanoethyl) (methyl)carba- moyl)-6- oxodecahydro- pyrrolo[1,2- a] [1,5]diazocin- 5- yl)carbamoyl) benzo[b]thio- phen-5- yl)difluorometh- yl)phosphon- ic acid | 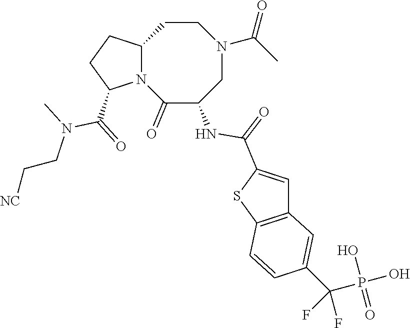 [SMILES: CC(=O)N1CC[C@H]2CC[C@@H](C(=O)N(C)CCC#N)N2C(=O)[C@@H](NC(=O)c2cc3cc(C(F)(F)P(=O)(O)O)ccc3s2)C1] | 624.2 [M − H]⁻ | ¹H NMR (400 MHz, DMSO-d₆) δ 9.09-8.57 (m, 1H), 8.31 (m, 1H), 8.15-8.04 (m, 2H), 7.60 (m, 1H), 5.00 (s, 1H), 4.79 (m, 1H), 4.28 (s, 1H), 3.91 (m, 1H), 3.84 (s, 1H), 3.69 (m, 2H), 3.44-3.31 (m, 3H), 3.13 (m, 2H), 3.09-3.01 (m, 1H), 2.88 (s, 1H), 2.69 (m, 1H), 2.13 (d, J = 25.2 Hz, 3H), 2.05 (dd, J = 15.7, 7.1 Hz, 1H), 1.93 (s, 1H), 1.86- 1.65 (m, 3H). |
| 91 | ((2- (((5S,8S,10aR)- 3-acetyl- 8-((2- hydroxy-2- methylpropyl) (methyl)car- bamoyl)-6- oxodecahydro- pyrrolo[1,2- a][1,5]diazocin- 5- yl)carbamoyl) benzo[b]thio- phen-5- yl)difluorometh- yl)phosphon- ic acid | 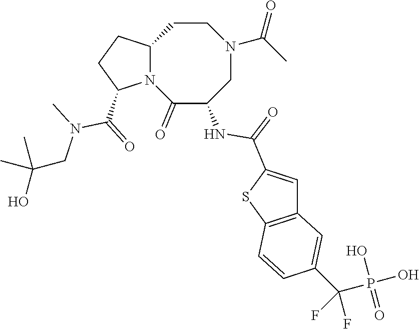 [SMILES: CC(=O)N1CC[C@H]2CC[C@@H](C(=O)N(C)CC(C)(C)O)N2C(=O)[C@@H](NC(=O)c2cc3cc(C(F)(F)P(=O)(O)O)ccc3s2)C1] | 643.0 [M − H]⁻ | ¹H NMR (400 MHz, DMSO-d₆) δ 9.06-8.58 (m, 1H), 8.36-8.26 (m, 1H), 8.09 (m, 2H), 7.58 (m, 1H), 4.94 (m, 1H), 4.80 (m, 1H), 4.47 (s, 1H), 4.27 (m, 1H), 3.83 (m, 1H), 3.73-3.61 (m, 2H), 3.16 (m, 3H), 3.08- 3.00 (m, 2H), 2.89 (m, 1H), 2.12 (m, 3H), 2.03 (m, 1H), 1.94-1.60 (m, 4H), 1.49 (m, 1H), 1.14 (m, 3H), 1.04 (m, 3H). |
| 92 | ((2- (((5S,8S,10aR)- 3-acetyl- 8-((2-cyano- 2- methylpropyl) (methyl)car- bamoyl)-6- oxodecahydro- pyrrolo[1,2- a][1,5]diazocin- 5- yl)carbamoyl) benzo[b]thio- phen-5- yl)difluorometh- yl)phosphon- ic acid | 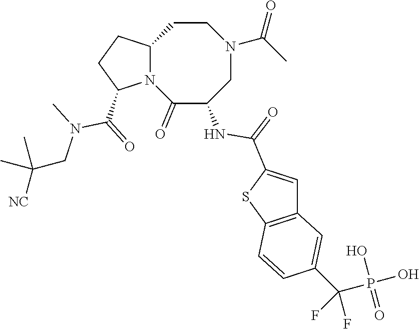 [SMILES: CC(=O)N1CC[C@H]2CC[C@@H](C(=O)N(C)CC(C)(C)C#N)N2C(=O)[C@@H](NC(=O)c2cc3cc(C(F)(F)P(=O)(O)O)ccc3s2)C1] | 652.0 [M − H]⁻ | ¹H NMR (400 MHz, DMSO-d₆) δ 8.81-8.60 (m, 1H), 8.32 (s, 1H), 8.18-8.02 (m, 2H), 7.58 (m, 1H), 5.02 (s, 1H), 4.81 (m, 1H), 4.29 (s, 1H), 3.87 (m, 2H), 3.54 (m, 2H), 3.24 (m, 3H), 3.13-2.90 (m, 2H), 2.15- 2.08 (m, 3H), 2.06 (m, 2H), 1.83-1.62 (m, 4H), 1.41-1.15 (m, 6H). |
| 93 | ((2- (((5S,8S,10aR)- 3-acetyl- 8-(methyl(1- (methylsulfo- nyl)piperidin- 4- yl)carbamoyl)- 6- oxodecahydro- pyrrolo[1,2- a][1,5]diazocin- 5-yl)carbamoyl) benzo[b]thio- phen-5- yl)difluorometh- yl)phosphon- ic acid | 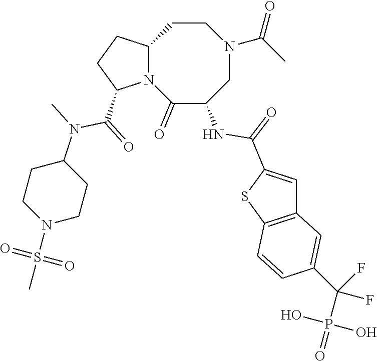 [SMILES: CC(=O)N1CC[C@H]2CC[C@@H](C(=O)N(C)C3CCN(S(C)(=O)=O)CC3)N2C(=O)[C@@H](NC(=O)c2cc3cc(C(F)(F)P(=O)(O)O)ccc3s2)C1] | 734.0 [M − H]⁻ | ¹H NMR (600 MHz, DMSO-d₆) δ 9.02-8.54 (m, 1H), 8.36-8.27 (m, 1H), 8.11 (m, 1H), 8.07 (m, 1H), 7.58 (m, 1H), 5.02-4.84 (m, 1H), 4.81- 4.71 (m, 1H), 4.38- 4.22 (m, 2H), 3.98-3.93 (m, 1H), 3.89-3.86 (m, 1H), 3.70 (s, 1H), 3.63 (s, 1H), 2.91 (m, 2H), 2.87-2.83 (m, 3H), 2.80- 2.74 (m, 1H), 2.69 (m, 1H), 2.37-2.27 (m, 2H), 2.17 (d, J = 6.7 Hz, 3H), 2.08 (s, 1H), 2.06- 1.92 (m, 2H), 1.89-1.79 (m, 2H), 1.77-1.61 (m, 5H), 1.56-1.50 (m, 1H). |
| 94 | ((2- (((5S,8S,10aR)- 3-acetyl- 8-((2- (methylsulfo- nyl)pyrimidin- 5- yl)carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 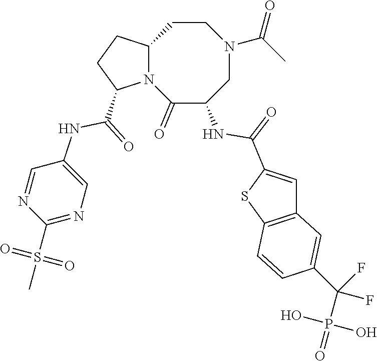 [SMILES: CC(=O)N1CC[C@H]2CC[C@@H](C(=O)Nc3cnc(S(C)(=O)=O)nc3)N2C(=O)[C@@H](NC(=O)c2cc3cc(C(F)(F)P(=O)(O)O)ccc3s2)C1] | 715.0 [M + H]⁺ | ¹H NMR (500 MHz, DMSO-d₆) δ 11.08 (m, 1H), 9.29-9.04 (m, 2H), 8.38-8.25 (m, 1H), 8.25-8.01 (m, 3H), 7.83-7.51 (m, 2H), 5.01 (m, 1H), 4.52 (m, 1H), 4.34 (m, 1H), 3.80-3.69 (m, 2H), 3.55- 3.47 (m, 2H), 3.07 (m, 2H), 2.28 (m, 1H), 2.18- 2.03 (m, 3H), 1.96 (m, 1H), 1.88-1.72 (m, 2H), 1.15 (m, 2H). |
| 95 | ((1- (((5S,8S,10aR)- 3-acetyl- 6-oxo-8-(3- (pyridin-4- yloxy)pyrroli- dine-1- carbonyl)deca- hydropyrrolo [1,2- a][1,5]diazocin- 5- yl)carbamoyl) benzo[b]thio- phen-5- yl)difluorometh- yl)phosphon- ic acid | 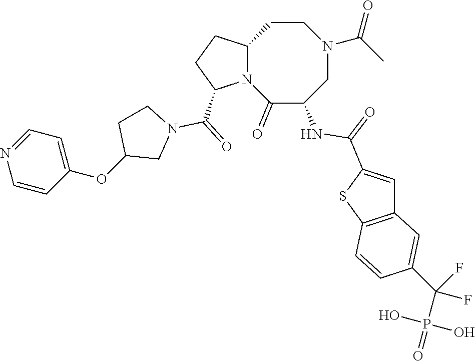 [SMILES: CC(=O)N1CC[C@H]2CC[C@@H](C(=O)N3CCC(Oc4ccncc4)C3)N2C(=O)[C@@H](NC(=O)c2cc3cc(C(F)(F)P(=O)(O)O)ccc3s2)C1] | 706.0 [M + H]⁺ | ¹H NMR (500 MHz, DMSO-d₆) δ 8.64-8.44 (m, 2H), 8.39-8.20 (m, 1H), 8.08 m, 3H), 7.77- 7.06 (m, 3H), 5.42-5.20 (m, 1H), 4.93 (m, 1H), 4.71-4.47 (m, 2H), 4.24 (s, 1H), 4.08-3.96 (m, 2H), 3.89-3.81 (m, 2H), 3.70 (s, 2H), 3.59 (m, 2H), 3.09 (m, 1H), 2.33-2.16 (m, 2H), 2.16- 2.07 (m, 3H), 2.05 (m, 1H), 1.94 (s, 1H), 1.87- 1.61 (m, 3H), 1.14 (m, 1H). |
| 96 | ((2- (((5S,8S,10aR)- 8-([1,1′- biphenyl]-4- yl(methyl)car- bamoyl)-3- acetyl-6- oxodecahydro- pyrrolo[1,2- a][1,5]diazocin- 5- yl)carbamoyl) benzo[b]thio- phen-5- yl)difluorometh- yl)phosphon- ic acid | 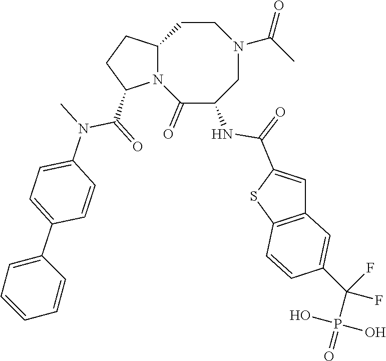 [SMILES: CC(=O)N1CC[C@H]2CC[C@@H](C(=O)N(C)c3ccc(-c4ccccc4)cc3)N2C(=O)[C@@H](NC(=O)c2cc3cc(C(F)(F)P(=O)(O)O)ccc3s2)C1] | 725.0 [M + H]⁺ | ¹H NMR (500 MHz, DSO-d₆) δ 9.05-8.42 (m, 1H), 8.33 (m, 1H), 8.11 (m, 2H), 7.78-7.63 (m, 4H), 7.58 (m, 1H), 75.1-7.31 (m, 5H), 4.86 (m, 1H), 4.31 (s, 1H), 4.16 (s, 1H), 3.92-3.80 (m, 2H), 3.17 (m, 3H), 3.06 (m, 1H), 2.14 (m, 3H), 1.91 (m, 4H), 1.73 (s, 1H), 1.62 (s, 1H). |
| 97 | ((2- (((5S,8S,10aR)- 3-acetyl- 6-oxo-8-((S)- 3- phenylpyrroli- dine-1- carbonyl)deca- hydropyrrolo [1,2- a][1,5]diazocin- 5- yl)carbamoyl) benzo[b]thio- phen-5- yl)difluorometh- yl)phosphon- ic acid | 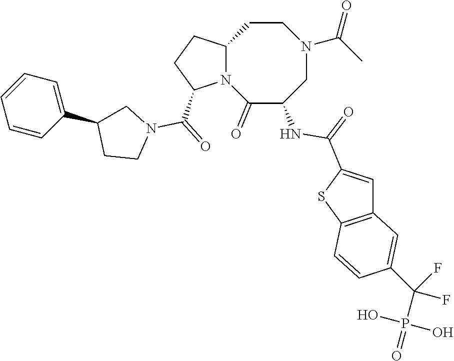 [SMILES: CC(=O)N1CC[C@H]2CC[C@@H](C(=O)N3CC[C@@H](c4ccccc4)C3)N2C(=O)[C@@H](NC(=O)c2cc3cc(C(F)(F)P(=O)(O)O)ccc3s2)C1] | 689.2 [M + H]⁺ | ¹H NMR (400 MHz, DMSO-d₆) δ 8.35 (m, 1H), 8.18-8.09 (m, 2H), 7.61 (m, 1H), 7.42- 6.96 (m, 6H), 5.06- 4.84 (m, 1H), 4.64 (m, 1H), 4.29 (s, 1H), 4.03 (m, 1H), 3.91 (m, 1H), 3.77 (m, 2H), 3.68 (m, 1H), 3.59 (m, 1H), 3.50 (m, 1H), 2.36 (m, 2H), 2.17 (m, 4H), 2.11-1.93 (m, 3H), 1.93-1.73 (m, 4H). |
| 98 | ((4-((E)-3- (((5S,8S,10aR)- 3-acetyl- 8- (methyl(phenyl) carbamoyl)- 6- oxodecahydro- pyrrolo[1,2- a][1,5]diazocin- 5- yl)amino)-3- oxoprop-1- en-1- yl)phenyl)diflu- oromethyl) phosphonic acid | 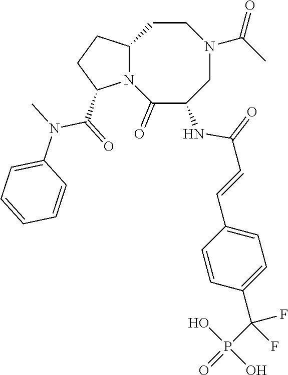 [SMILES: CC(=O)N1CC[C@H]2CC[C@@H](C(=O)N(C)c3ccccc3)N2C(=O)[C@@H](NC(=O)/C=C/c2ccc(C(F)(F)P(=O)(O)O)cc2)C1] | 619.0 [M + H]⁺ | ¹H NMR (500 MHz, DMSO-d₆) δ 8.18 (s, 1H), 7.65 (m, 2H), 7.54 (m, 2H), 7.50-7.36 (m, 6H), 7.07 (m, 1H), 4.75 (s, 1H), 4.20 (s, 1H), 4.10 (s, 1H), 3.81 (d, J = 15.2 Hz, 2H), 3.16 (s, 3H), 3.10-3.00 (m, 2H), 2.20 (s, 3H), 1.97 (s, 1H), 1.82 (s, 3H), 1.69-1.52 (m, 2H). |
| 99 | ((2- (((5S,8S,10aR)- 3-acetyl- 6-oxo-8-(3- phenoxyazeti- dine-1- carbonyl)deca- hydropyrrolo [1,2- a][1,5]diazocin- 5- yl)carbamoyl)- 1H-indol-5- yl)difluorometh- yl)phosphon- ic acid | 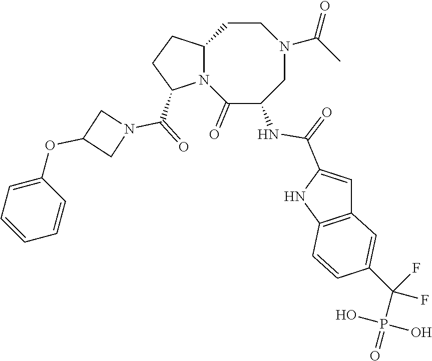 [SMILES: CC(=O)N1CC[C@H]2CC[C@@H](C(=O)N3CC(Oc4ccccc4)C3)N2C(=O)[C@@H](NC(=O)c2cc3cc(C(F)(F)P(=O)(O)O)ccc3[nH]2)C1] | 674.0 [M + H]⁺ | ¹H NMR (600 MHz, DMSO-d₆) δ 11.82 (m, 1H), 8.72-8.33 (m, 1H), 7.82 (m, 1H), 7.47 (m, 1H), 7.36 (m, 1H), 7.30 (m, 3H), 7.04-6.92 (m, 1H), 6.90-6.79 (m, 2H), 5.18-4.90 (m, 2H), 4.87-4.56 (m, 1H), 4.41-4.13 (m, 4H), 3.87-3.73 (m, 2H), 3.67 (s, 1H), 3.56 (s, 1H), 2.24-2.15 (m, 3H), 2.11-2.06 (m, 1H), 2.05-1.88 (m, 2H), 1.88-1.68 (m, 3H). |
| 100 | ((2- (((5S,8S,10aR)- 3-acetyl- 6-oxo-8-(3- phenylazetidine- 1- carbonyl)deca- hydropyrrolo [1,2- a][1,5]diazocin- 5- yl)carbamoyl)- 1H-indol-5- yl)difluorometh- yl)phosphon- ic acid | 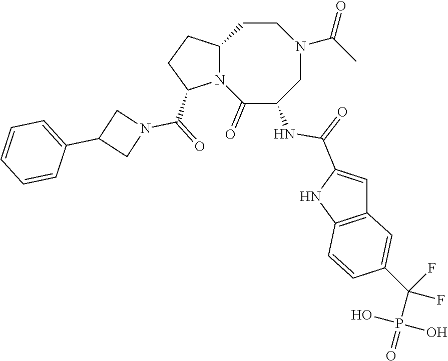 [SMILES: CC(=O)N1CC[C@H]2CC[C@@H](C(=O)N3CC(c4ccccc4)C3)N2C(=O)[C@@H](NC(=O)c2cc3cc(C(F)(F)P(=O)(O)O)ccc3[nH]2)C1] | 658.0 [M + H]⁺ | ¹H NMR (400 MHz, DMSO-d₆) δ 11.94 (m, 1H), 8.41 (m, 1H), 7.84 (s, 1H), 7.50 (m, 1H), 7.43-7.26 (m, 8H), 4.99 (m, 1H), 4.90-4.49 (m, 1H), 4.47-4.21 (m, 4H), 3.87 (m, 3H), 3.70 (s, 1H), 3.62 (s, 1H), 2.19 (m, 3H), 2.10 (m, 1H), 1.99 (s, 2H), 1.79 (m, 3H). |
| 101 | ((2- (((5S,8S,10aR)- 3-acetyl- 8-((4- ethoxyphenyl) (methyl)car- bamoyl)-6- oxodecahydro- pyrrolo[1,2- a][1,5]diazocin- 5- yl)carbamoyl)- 1H-indol-5- yl)difluorometh- yl)phosphon- ic acid | 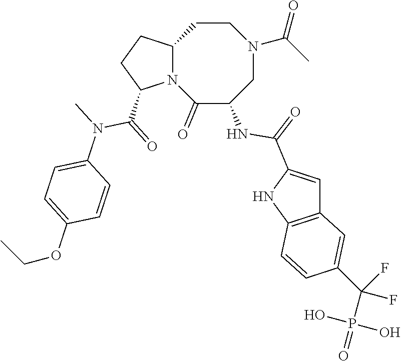 [SMILES: CCOc1ccc(N(C)C(=O)[C@@H]2CC[C@@H]3CCN(C(C)=O)C[C@H](NC(=O)c4cc5cc(C(F)(F)P(=O)(O)O)ccc5[nH]4)C(=O)N32)cc1] | 676.4 [M + H]⁺ | ¹H NMR (400 MHz, DMSO-d₆) δ 11.86 (m, 1H), 7.84 (s, 1H), 7.49 (m, 1H), 7.32 (m, 5H), 6.97 (m, 2H), 4.94 (s, 1H), 4.24 (m, 1H), 4.17 (s, 1H), 4.04-3.99 (m, 2 H), 3.87 (m, 1H), 3.78 (m, 1H), 3.11 (m, 3H), 2.21 (s, 3H), 2.11 (s, 1H), 1.95 (s, 1H), 1.82 (m, 4H), 1.64 (m, 2H), 1.30 (m, 3H). |
| 102 | ((2- (((5S,8S,10aR)- 3-acetyl- 8-((4-(2- (dimethylami- no)ethoxy)phe- nyl)(methyl) carbamoyl)- 6- oxodecahydro- pyrrolo[1,2- a][1,5]diazocin- 5- yl)carbamoyl)- 1H-indol-5- yl)difluorometh- yl)phosphon- ic acid | 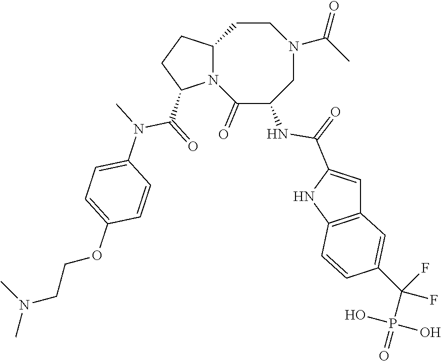 [SMILES: CC(=O)N1CC[C@H]2CC[C@@H](C(=O)N(C)c3ccc(OCCN(C)C)cc3)N2C(=O)[C@@H](NC(=O)c2cc3cc(C(F)(F)P(=O)(O)O)ccc3[nH]2)C1] | 719.6 [M + H]⁺ | ¹H NMR (400 MHz, DMSO-d₆) δ 11.90 (m, 1H), 7.76 (s, 1H), 7.43 (m, 3H), 7.23 (m, 3H), 6.84 (m, 1H), 4.94 (m, 1H), 4.29-4.00 (m, 4H), 3.82 (m, 2H), 3.59 (m, 1H), 3.39 (s, 2H), 3.08 (m, 3H), 2.77 (s, 6H), 2.21 (s, 3H), 2.11 (s, 1H), 1.98 (s, 1H), 1.90-1.73 (m, 4H), 1.60 (s, 1H). |
| 103 | ((2- (((5S,8S,10aR)- 3-acetyl- 6-oxo-8-((4- (2- oxopyridin- 1(2H)- yl)phenyl)carba- moyl)deca- hydropyrrolo [1,2- a][1,5]diazocin- 5- yl)carbamoyl)- 1H-indol-5- yl)difluorometh- yl)phosphon- ic acid | 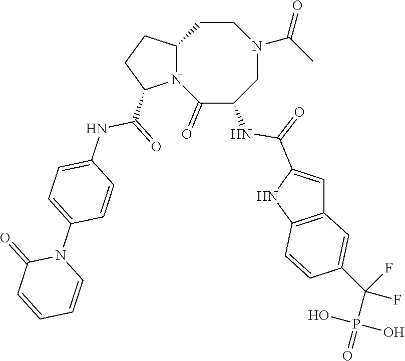 [SMILES: CC(=O)N1CC[C@H]2CC[C@@H](C(=O)Nc3ccc(-n4ccccc4=O)cc3)N2C(=O)[C@@H](NC(=O)c2cc3cc(C(F)(F)P(=O)(O)O)ccc3[nH]2)C1] | 711.2 [M + H]⁺ | ¹H NMR (600 MHz, DMSO-d₆) δ 12.01- 11.74 (m, 1H), 10.32 (m, 1H), 7.81 (s, 1H), 7.72- 7.65 (m, 2H), 7.58 (m, 1H), 7.48 (m, 2H), 7.38- 7.34 (m, 1H), 7.34- 7.06 (m, 4H), 6.45 (m, 1H), 6.31-6.26 (m, 1H), 5.09-4.90 (m, 1H), 4.51 (m, 1H), 4.33 (m, 2H), 3.85 (m, 1H), 2.31-2.27 (m, 1H), 2.19 (s, 3H), 2.11 (s, 1H), 2.10-1.93 (m, 4H), 1.81- 1.73 (m, 2H). |
| 104 | ((2- (((5S,8S,10aR)- 3-acetyl- 8-(2- benzylazetidine- 1- carbonyl)-6- oxodecahydro- pyrrolo[1,2- a][1,5]diazocin- 5- yl)carbamoyl)- 1H-indol-5- yl)difluorometh- yl)phosphon- ic acid | 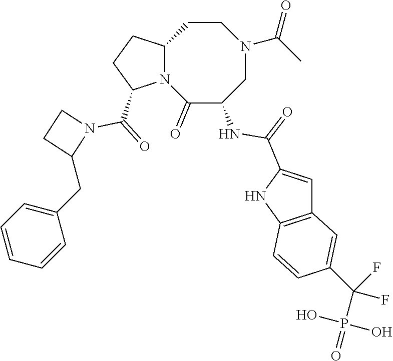 [SMILES: CC(=O)N1CC[C@H]2CC[C@@H](C(=O)N3CCC3Cc3ccccc3)N2C(=O)[C@@H](NC(=O)c2cc3cc(C(F)(F)P(=O)(O)O)ccc3[nH]2)C1] | 672.2 [M + H]⁺ | ¹H NMR (600 MHz, DMSO-d₆) δ 12.06 (m, 1H), 8.45-8.26 (m, 1H), 7.86 (m, 1H), 7.71- 7.52 (m, 1H), 7.44 (m, 2H), 7.33-6.89 (m, 5H), 4.46 (m, 1H), 4.35- 4.07 (m, 2H), 4.01- 3.81 (m, 1H), 3.79-3.44 (m, 5H), 3.15-2.87 (m, 5H), 2.25-2.07 (m, 3H), 2.05-1.67 (m, 4H), 1.13 (m, 2H). |
| 105 | ((2- (((5S,8S,10aR)- 3-acetyl- 8-((3-cyano- 2-oxo-1- phenyl-1,2- dihydropyridin- 4- yl)carbamoyl)- 6- oxodecahydro- pyrrolo[1,2- a][1,5]diazocin- 5- yl)carbamoyl)- 1H-indol-5- yl)difluorometh- yl)phosphon- ic acid | 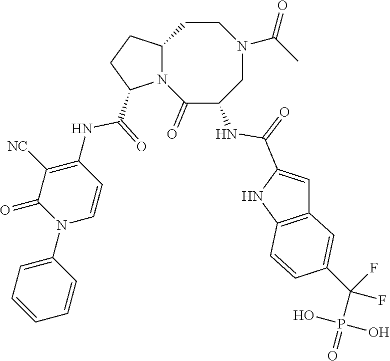 [SMILES: CC(=O)N1CC[C@H]2CC[C@@H](C(=O)Nc3ccn(-c4ccccc4)c(=O)c3C#N)N2C(=O)[C@@H](NC(=O)c2cc3cc(C(F)(F)P(=O)(O)O)ccc3[nH]2)C1] | 736.2 [M + H]⁺ | ¹H NMR (400 MHz, DMSO-d₆) δ 11.76 (m, 1H), 10.68 (s, 1H), 8.44 (m, 1H), 7.97 (m, 1H), 7.82 (s, 1H), 7.62-7.21 (m, 8H), 7.03 (m, 1H), 5.06 (m, 1H), 4.77 (m, 1H), 4.36 (s, 2H), 3.82 (m, 1H), 2.15 (s, 3H), 2.10 (s, 1H), 1.93 (s, 2H), 1.77 (s, 3H). |
| 106 | ((2- (((5S,8S,10aR)- 3-acetyl- 8-((S)-2- benzylpiperi- dine-1- carbonyl)-6- oxodecahydro- pyrrolo[1,2- a][1,5]diazocin- 5- yl)carbamoyl)- 1H-indol-5- yl)difluorometh- yl)phosphon- ic acid | 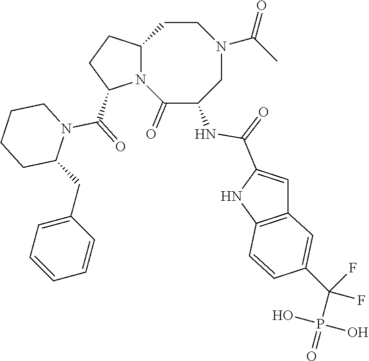 [SMILES: CC(=O)N1CC[C@H]2CC[C@@H](C(=O)N3CCCC[C@H]3Cc3ccccc3)N2C(=O)[C@@H](NC(=O)c2cc3cc(C(F)(F)P(=O)(O)O)ccc3[nH]2)C1] | 700.4 [M + H]⁺ | ¹H NMR (600 MHz, DMSO-d₆) δ 11.81 (m, 1H), 8.62-8.19 (m, 1H), 7.80 (m, 1H), 7.44 (m, 1H), 7.36 (m, 1H), 7.28 (m, 1H), 7.27-7.18 (m, 4H), 7.15 (m, 1H), 5.05-4.72 (m, 3H), 4.39- 4.23 (m, 1H), 4.17 (m, 1H), 3.92-3.88 (m, 1H), 3.80 (m, 1H), 3.74 (m, 1H), 3.22-3.17 (m, 2H), 2.88 (m, 1H), 2.80 (m, 1H), 2.24 (m, 3H), 2.12-2.04 (m, 2H), 2.04- 1.95 (m, 1H), 1.88 (s, 1H), 1.77 (m, 1H), 1.66 (m, 1H), 1.58 (m, 1H), 1.54-1.43 (m, 3H), 1.35- 1.12 (m, 2H). |
| 107 | ((2- (((5S,8S,10aR)- 3-acetyl- 6-oxo-8-((S)- 3- phenylpiperi- dine-1- carbonyl)deca- hydropyrrolo [1,2- a][1,5]diazocin- 5- yl)carbamoyl)- 1H-indol-5- yl)difluorometh- yl)phosphon- ic acid | 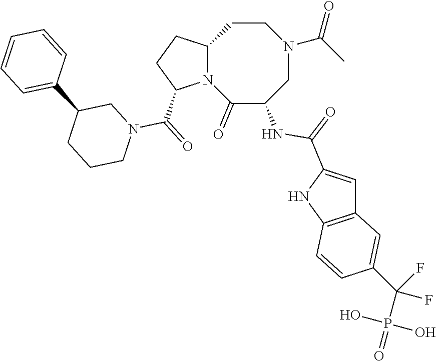 [SMILES: CC(=O)N1CC[C@H]2CC[C@@H](C(=O)N3CCC[C@@H](c4ccccc4)C3)N2C(=O)[C@@H](NC(=O)c2cc3cc(C(F)(F)P(=O)(O)O)ccc3[nH]2)C1] | 686.2 [M + H]⁺ | ¹H NMR (600 MHz, DMSO-d₆) δ 11.99- 10.97 (m, 1H), 8.11- 8.08 (m, 1H), 7.81 (m, 1H), 7.41 (m, 2H), 7.33- 7.19 (m, 5H), 6.93- 6.90 (m, 1H), 5.00-4.83 (m, 1H), 4.40 (m, 1H), 4.26 (s, 1H), 4.05 (m, 1H), 3.89 (m, 1H), 3.74 (s, 1H), 3.42 (m, 2H), 3.22-3.04 (m, 4H), 2.89 (s, 1H), 2.35-2.24 (m, 1H), 2.23-2.07 (m, 3H), 2.05-1.80 (m, 4H), 1.77-1.61 (m, 3H), 1.44 (m, 1H). |
| 108 | ((2- (((5S,8S,10aR)- 3-acetyl- 8-((2- cyanophenyl) (methyl)carba- moyl)-6- oxodecahydro- pyrrolo[1,2- a][1,5]diazocin- 5- yl)carbamoyl)- 1H-indol-5- yl)difluorometh- yl)phosphon- ic acid | 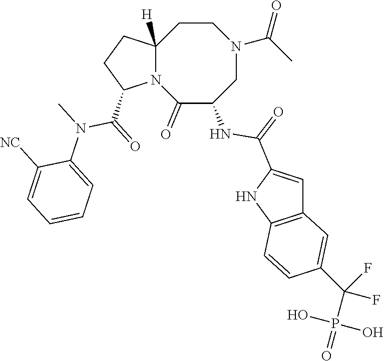 [SMILES: CC(=O)N1CC[C@H]2CC[C@@H](C(=O)N(C)c3ccccc3C#N)N2C(=O)[C@@H](NC(=O)c2cc3cc(C(F)(F)P(=O)(O)O)ccc3[nH]2)C1] | 657.2 [M + H]⁺ | ¹HNMR (400 MHz, METHANOL-d₄) δ 7.93-7.82 (m, 2H), 7.80-7.70 (m, 1H), 7.63-7.39 (m, 4H), 7.24 (s, 1H), 5.18-4.98 (m, 1H), 4.34- 4.26 (m, 1H), 4.07- 3.89 (m, 1H), 3.88-3.68 (m, 2H), 3.67-3.60 (m, 1H), 3.60-3.44 (m, 1H), 3.30-3.23 (m, 3H), 2.32- 2.24 (m, 3H), 2.18- 1.92 (m, 5H), 1.87-1.73 (m, 1H) |
| 109 | ((2- (((5S,8S,10aR)- 3-acetyl- 8- (methyl(phenyl) carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 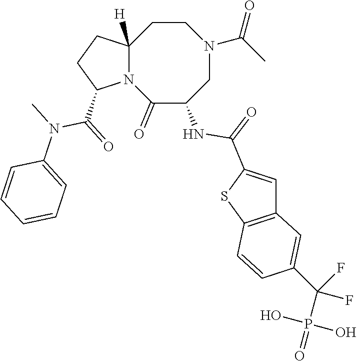 [SMILES: CC(=O)N1CC[C@H]2CC[C@@H](C(=O)N(C)c3ccccc3)N2C(=O)[C@@H](NC(=O)c2cc3cc(C(F)(F)P(=O)(O)O)ccc3s2)C1] | 649.3 [M + H]⁺ | ¹HNMR (400 MHz, METHANOL-d₄) δ 8.18-8.12 (m, 1H), 8.10-7.91 (m, 2H), 7.72-7.64 (m, 1H), 7.50-7.26 (m, 5H), 5.07-4.92 (m, 1H), 4.31-4.19 (m, 1H), 4.01 (dd, J = 3.6, 14 Hz, 1H), 3.91-3.78 (m, 1H), 3.69- 3.48 (m, 2H), 3.28- 3.21 (m, 3H), 2.34-2.23 (m, 3H), 2.21-1.89 (m, 6H), 1.84-1.70 (m, 1H) |
| 110 | ((2- (((5S,8S,10aR)- 3-acetyl- 8- (methyl(phenyl) carbamoyl)- 6- oxodecahydro- pyrrolo[1,2- a][1,5]diazocin- 5- yl)carbamoyl) thieno[2,3- b]pyridin-5- yl)difluorometh- yl)phosphon- ic acid | 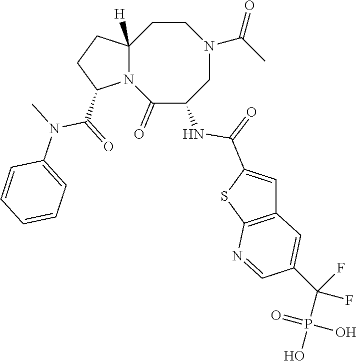 [SMILES: CC(=O)N1CC[C@H]2CC[C@@H](C(=O)N(C)c3ccccc3)N2C(=O)[C@@H](NC(=O)c2cc3cc(C(F)(F)P(=O)(O)O)cnc3s2)C1] | 650.3 [M + H]⁺ | ¹H NMR (400 MHz, METHANOL-d₄) δ 8.88-8.67 (m, 1H), 8.56-8.37 (m, 1H), 8.18-7.98 (m, 1H), 7.56-7.25 (m, 5H), 5.13-5.01 (m, 1H), 4.54-4.15 (m, 2H), 4.07- 3.95 (m, 1H), 3.91- 3.74 (m, 1H), 3.73-3.44 (m, 2H), 3.28-3.22 (m, 3H), 2.43-2.23 (m, 3H), 2.09-1.86 (m, 5H), 1.84- 1.64 (m, 1H) |
| 111 | ((7- (((5S,8S,10aR)- 3-acetyl- 8- (methyl(phenyl) carbamoyl)- 6- oxodecahydro- pyrrolo[1,2- a][1,5]diazocin- 5- yl)carbamoyl) naphthalen-2- yl)difluorometh- yl)phosphon- ic acid | 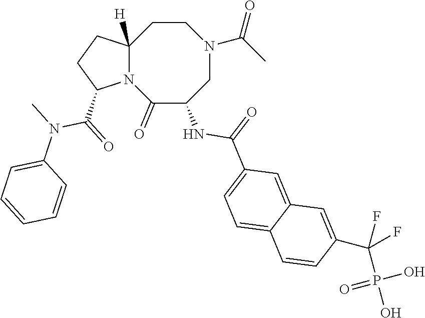 [SMILES: CC(=O)N1CC[C@H]2CC[C@@H](C(=O)N(C)c3ccccc3)N2C(=O)[C@@H](NC(=O)c2ccc3ccc(C(F)(F)P(=O)(O)O)cc3c2)C1] | 643.5 [M + H]⁺ | ¹H NMR (400 MHz, METHANOL-d₄) δ 8.53 (s, 1H), 8.28 (s, 1H), 8.07-7.89 (m, 3H), 7.86- 7.79 (m, 1H), 7.53- 7.36 (m, 5H), 5.16-4.95 (m, 1H), 4.49-4.26 (m, 2H), 4.12-3.78 (m, 2H), 3.76-3.43 (m, 2H), 3.29- 3.21 (m, 3H), 2.35 (s, 2H), 2.27 (s, 1H), 2.12- 1.86 (m, 5H), 1.82-1.69 (m, 1H) |
| 112 | ((6- (((5S,8S,10aR)- 3-acetyl- 8-(methyl (phenyl)- carbamoyl)- 6- oxodecahydro- pyrrolo[1,2- a][1,5]diazocin- 5- yl)carbamoyl) naphthalen- 2- yl)difluorometh- yl)phosphon- ic acid | 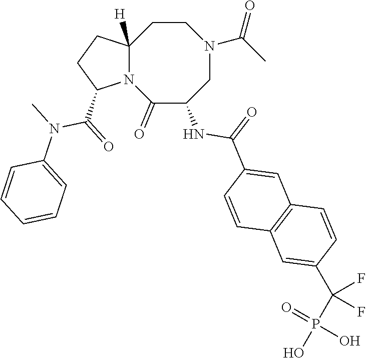 [SMILES: CC(=O)N1CC[C@H]2CC[C@@H](C(=O)N(C)c3ccccc3)N2C(=O)[C@@H](NC(=O)c2ccc3cc(C(F)(F)P(=O)(O)O)ccc3c2)C1] | 643 [M + H]⁺ | ¹H NMR (400 MHz, METHANOL-d₄) δ 8.54-8.45 (m, 1H), 8.25-8.17 (m, 1H), 8.13-7.93 (m, 3H), 7.82 (d, J = 9.4 Hz, 1H), 7.54-7.23 (m, 5H), 5.11-4.98 (m, 1H), 4.47-4.25 (m, 2H), 4.10- 3.99 (m, 1H), 3.91- 3.82 (m, 1H), 3.74-3.51 (m, 2H), 3.29-3.22 (m, 3H), 2.37-2.24 (m, 3H), 2.09-1.72 (m, 6H) |
| 113 | (6- (((5S,8S,10aR)- 3-acetyl- 8- (methyl(phenyl) carbamoyl)- 6- oxodecahydro- pyrrolo[1,2- a][1,5]diazocin- 5- yl)carbamoyl) naphthalen- 2- yl)phosphonic acid | 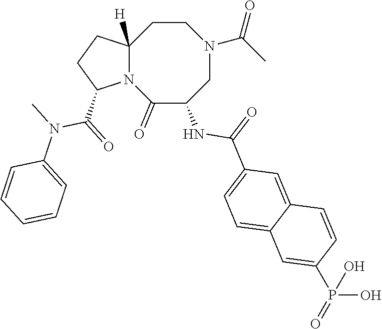 [SMILES: CC(=O)N1CC[C@H]2CC[C@@H](C(=O)N(C)c3ccccc3)N2C(=O)[C@@H](NC(=O)c2ccc3cc(P(=O)(O)O)ccc3c2)C1] | 593.4 [M + H]⁺ | ¹H NMR (400 MHz, METHANOL-d₄) δ 8.51-8.32 (m, 2H), 8.12- 7.81 (m, 4H), 7.53-7.26 (m, 5H), 5.13-4.99 (m, 1H), 4.48-4.23 (m, 2H), 4.04 (dd, J = 3.6, 14.4 Hz, 1H), 3.93-3.81 (m, 1H), 3.71-3.49 (m, 2H), 3.29-3.17 (m, 3H), 2.37- 2.24 (m, 3H), 2.21- 1.88 (m, 5H), 1.86-1.71 (m, 1H), 1.35-1.16 (m, 1H) |
| 114 | (7- (((5S,8S,10aR)- 3-acetyl- 8- (methyl(phenyl) carbamoyl)- 6- oxodecahydro- pyrrolo[1,2- a][1,5]diazocin- 5- yl)carbamoyl) naphthalen- 2- yl)phosphonic acid | 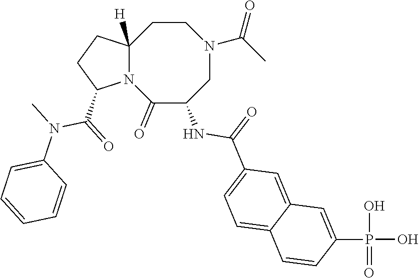 [SMILES: CC(=O)N1CC[C@H]2CC[C@@H](C(=O)N(C)c3ccccc3)N2C(=O)[C@@H](NC(=O)c2ccc3ccc(P(=O)(O)O)cc3c2)C1] | 593.4 [M + H]⁺ | ¹H NMR (400 MHz, METHANOL-d₄) δ 8.59-8.43 (m, 2H), 8.09- 7.97 (m, 3H), 7.96-7.88 (m, 1H), 7.52-7.35 (m, 5H), 5.15-4.97 (m, 1H), 4.47-4.36 (m, 1H), 4.36 (s, 1H), 4.10-3.99 (m, 1H), 3.92-3.80 (m, 1H), 3.77-3.63 (m, 1H), 3.62- 3.52 (m, 1H), 3.28- 3.24 (m, 3H), 2.36-2.25 (m, 3H), 2.06-1.89 (m, 5H), 1.87-1.70 (m, 1H) |
| 115 | ((2- (((5S,8S,10aR)- 3-acetyl- 8- (methyl(phenyl) carbamoyl)- 6- oxodecahydro- pyrrolo[1,2- a][1,5]diazocin- 5- yl)carbamoyl) benzo[d]thia- zol-5- yl)difluorometh- yl)phosphon- ic acid | 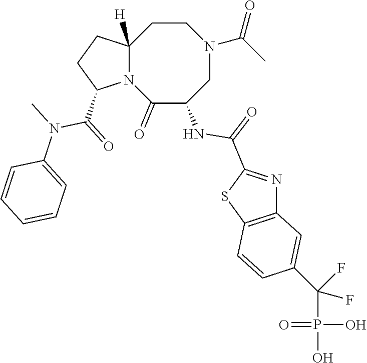 [SMILES: CC(=O)N1CC[C@H]2CC[C@@H](C(=O)N(C)c3ccccc3)N2C(=O)[C@@H](NC(=O)c2nc3cc(C(F)(F)P(=O)(O)O)ccc3s2)C1] | 650.1 [M + H]⁺ | ¹H NMR (400 MHz, METHANOL-d₄) δ 8.41 (s, 1H), 8.25-8.15 (m, 1H), 7.81 (d, J = 8.8 Hz, 1H), 7.59-7.40 (m, 5H), 5.04-4.93 (m, 1H), 4.50- 4.40 (m, 1H), 4.32- 4.15 (m, 2H), 4.03-3.92 (m, 1H), 3.61-3.40 (m, 2H), 3.27 (s, 3H), 2.39 (s, 3H), 2.19-2.11 (m, 1H), 2.04-1.88 (m, 4H), 1.81-1.68 (m, 1H) |
| 116 | ((2- (((5S,8S,10aR)- 3-acetyl- 8-(4- carbamoyl-6- phenyl- 1,2,3,4- tetrahydroquino- line-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 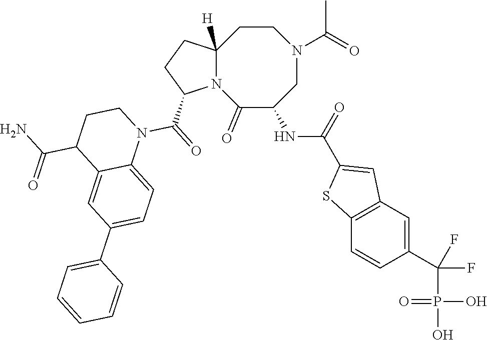 [SMILES: CC(=O)N1CC[C@H]2CC[C@@H](C(=O)N3CCC(C(N)=O)c4cc(-c5ccccc5)ccc43)N2C(=O)[C@@H](NC(=O)c2cc3cc(C(F)(F)P(=O)(O)O)ccc3s2)C1] | 794.1 [M + H]⁺ | ¹H NMR (400 MHz, METHANOL-d₄) δ 8.23-8.16 (m, 1H), 8.15- 8.08 (m, 1H), 8.00-7.91 (m, 1H), 7.76-7.68 (m, 1H), 7.64-7.50 (m, 5H), 7.42 (t, J = 7.6 Hz, 2H), 7.37-7.28 (m, 1H), 5.17- 5.08 (m, 2H), 4.54- 4.31 (m, 1H), 4.01-3.84 (m, 3H), 3.80-3.63 (m, 3H), 3.52-3.44 (m, 1H), 2.29 (s, 4H), 2.18 (d, J = 8.0 Hz, 3H), 2.05-1.87 (m, 2H), 1.32-1.28 (m, 2H) |
| 117 | ((7- (((5S,8S,10aR)- 3-acetyl- 8-((4-(tert- butyl)phenyl) (methyl)carba- moyl)-6- oxodecahydro- pyrrolo[1,2- a][1,5]diazocin- 5- yl)carbamoyl) naphthalen- 2- yl)difluorometh- yl)phosphon- ic acid | 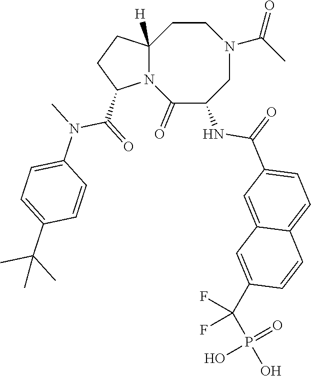 [SMILES: CC(=O)N1CC[C@H]2CC[C@@H](C(=O)N(C)c3ccc(C(C)(C)C)cc3)N2C(=O)[C@@H](NC(=O)c2ccc3ccc(C(F)(F)P(=O)(O)O)cc3c2)C1] | 699.5 [M + H]⁺ | ¹H NMR (400 MHz, METHANOL-d₄) δ 8.58-8.50 (m, 1H), 8.22 (s, 1H), 8.08- 7.96 (m, 3H), 7.84-7.78 (m, 1H), 7.53- 7.43 (m, 2H), 7.41- 7.30 (m, 2H), 5.12-5.07 (m, 1H), 4.47-4.25 (m, 2H), 4.24-4.01 (m, 1H), 3.90-3.84 (m, 1H), 3.60- 3.58 (m, 2H), 3.27- 3.21 (m, 3H), 2.37-2.24 (m, 3H), 2.07-1.89 (m, 5H), 1.82-1.67 (m, 1H), 1.33-1.28 (m, 9H) |
| 118 | ((2- (((5S,8S,10aR)- 3-acetyl- 8-(cinnolin- 6- yl(methyl)car- bamoyl)-6- oxodecahydro- pyrrolo[1,2- a][1,5]diazocin- 5- yl)carbamoyl) quinolin-7- yl)difluorometh- yl)phosphon- ic acid | 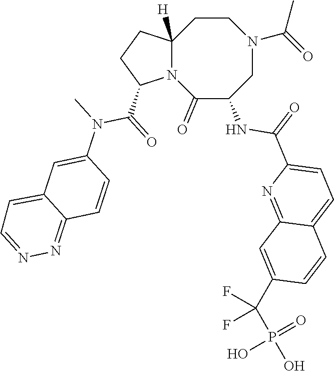 [SMILES: CC(=O)N1CC[C@H]2CC[C@@H](C(=O)N(C)c3ccc4nnccc4c3)N2C(=O)[C@@H](NC(=O)c2ccc3ccc(C(F)(F)P(=O)(O)O)cc3n2)C1] | 696.2 [M + H]⁺ | ¹H NMR (400 MHz, METHANOL-d₄) δ 8.61-8.15 (m, 6H), 7.23- 7.12 (m, 1H), 7.05-6.89 (m, 3H), 5.35-5.32 (m, 1H), 4.10-4.07 (m, 1H), 3.73-3.53 (m, 2H), 3.50-3.39 (m, 5H), 2.10-1.99 (m, 2H), 1.35-1.25 (m, 7H) |
| 119 | ((6- (((5S,8S,10aR)- 3-acetyl- 8-(cinnolin- 6- yl(methyl)car- bamoyl)-6- oxodecahydro- pyrrolo[1,2- a][1,5]diazocin- 5- yl)carbamoyl) isoquinolin- 3- yl)difluorometh- yl)phosphon- ic acid | 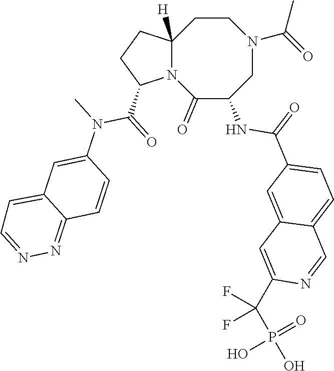 [SMILES: CC(=O)N1CC[C@H]2CC[C@@H](C(=O)N(C)c3ccc4nnccc4c3)N2C(=O)[C@@H](NC(=O)c2ccc3cnc(C(F)(F)P(=O)(O)O)cc3c2)C1] | 696.1 [M + H]⁺ | ¹H NMR (400 MHz, DMSO-d₆) δ 9.50 (s, 1H), 9.44-9.33 (m, 1H), 8.76-8.64 (m, 2H), 8.55 (d, J = 8.8 Hz, 1H), 8.36- 8.31 (m, 1H), 8.30-8.22 (m, 2H), 8.19 (d, J = 8.8 Hz, 1H), 8.11-8.03 (m, 1H), 8.02-7.93 (m, 1H), 5.07-4.90 (m, 1H), 4.27-4.24 (m, 1H), 3.75 (d, J = 12.4 Hz, 2H), 3.60 (d, J = 15.6 Hz, 1H), 3.49-3.40 (m, 2H), 3.38- 3.27 (m, 3H), 2.19 (s, 3H), 2.13 (s, 1H), 2.00- 1.89 (m, 3H), 1.84-1.76 (m, 1H), 1.73-1.63 (m, 1H) |
| 120 | ((7- (((5S,8S,10aR)- 3-acetyl- 8-(cinnolin- 6- yl(methyl)car- bamoyl)-6- oxodecahydro- pyrrolo[1,2- a][1,5]diazocin- 5- yl)carbamoyl) quinolin-2- yl)difluorometh- yl)phosphon- ic acid | 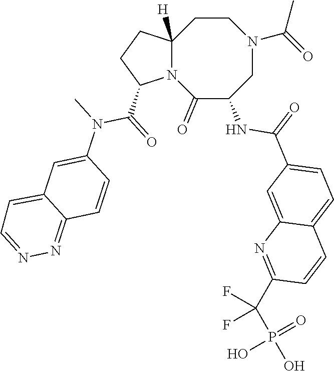 [SMILES: CC(=O)N1CC[C@H]2CC[C@@H](C(=O)N(C)c3ccc4nnccc4c3)N2C(=O)[C@@H](NC(=O)c2ccc3ccc(C(F)(F)P(=O)(O)O)nc3c2)C1] | 696.3 [M + H]⁺ | ¹H NMR (400 MHz, METHANOL-d₄) δ 9.33-9.21 (m, 1H), 8.64 (s, 1H), 8.52 (d, J = 9.2 Hz, 1H), 8.48-8.34 (m, 2H), 8.21 (d, J = 6.0 Hz, 1H), 8.12 (s, 1H), 8.09-8.05 (m, 1H), 8.04-8.01 (m, 2H), 8.01-7.96 (m, 1H), 7.98-7.88 (m, 1H), 5.19- 5.05 (m, 1H), 4.63- 4.47 (m, 1H), 4.45-4.30 (m, 1H), 4.08-3.98 (m, 1H), 3.95-3.83 (m, 1H), 3.81-3.63 (m, 3H), 3.43 (s, 3H), 2.33-2.27 (m, 3H), 2.13-2.03 (m, 3H), 2.02-1.91 (m, 2H), 1.83- 1.74 (m, 1H) |
| 121 | ((4-((E)-3- (((5S,8S,10aR)- 8-([1,1′- biphenyl]-4- ylcarbamoyl)- 3-acetyl-6- oxodecahydro- pyrrolo[1,2- a][1,5]diazocin- 5- yl)amino)-3- oxoprop-1- en-1- yl)phenyl)diflu- oromethyl) phosphonic acid | 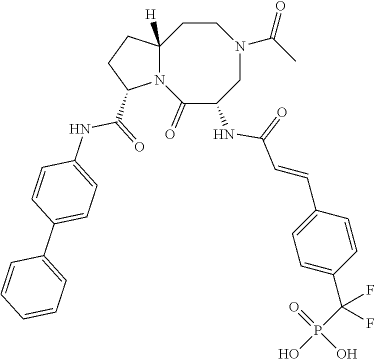 [SMILES: CC(=O)N1CC[C@H]2CC[C@@H](C(=O)Nc3ccc(-c4ccccc4)cc3)N2C(=O)[C@@H](NC(=O)/C=C/c2ccc(C(F)(F)P(=O)(O)O)cc2)C1] | | ¹H NMR (400 MHz, METHANOLd₄) δ 7.64- 7.70 (m, 4H) 7.54- 7.63 (m, 7H) 7.41 (t, J = 8.0 Hz, 2H) 7.26- 7.33 (m, 1H) 6.79 (d, J = 16.0 Hz, 1H) 5.11 (m, 1H), 4.56-4.65 (m, 1H) 4.35-4.45 (m, 1H) 3.82-3.88 (m, 1H) 3.63- 3.79 (m, 2H) 3.50 (m, 1H) 2.34-2.43 (m, 1H) 2.20-2.28 (m, 4H) 2.10- 2.20 (m, 1H) 1.95- 2.20 (m, 2H) 1.83-1.93 (m, 1H) |
| 122 | ((2- (((5S,8S,10aR)- 3-acetyl- 6-oxo-8-(3- (pyridin-3- yl)azetidine- 1- carbonyl)deca- hydropyrrolo [1,2- a][1,5]diazocin- 5- yl)carbamoyl) benzo[b]thio- phen-5- yl)difluorometh- yl)phosphon- ic acid | 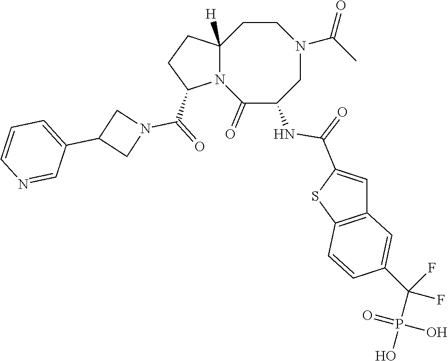 [SMILES: CC(=O)N1CC[C@H]2CC[C@@H](C(=O)N3CC(c4cccnc4)C3)N2C(=O)[C@@H](NC(=O)c2cc3cc(C(F)(F)P(=O)(O)O)ccc3s2)C1] | | ¹H NMR (400 MHz, METHANOL-d₄) δ 8.92-8.82 (m, 1H), 8.82- 8.66 (m, 2H), 8.20 (s, 1H), 8.12 (s, 1H), 8.10- 8.03 (m, 1H), 7.98 (d, J = 9.6 Hz, 1H), 7.73 (d, J = 8.0 Hz, 1H), 5.17-5.01 (m, 1H), 5.07-4.99 (m, 1H), 4.85-4.74 (m, 1H), 4.55-4.32 (m, 3H), 4.22- 4.08 (m, 2H), 3.99 (s, 1H), 3.92-3.70 (m, 2H), 3.69 (s, 1H), 2.32 (d, J = 2.8 Hz, 1H), 2.29-2.26 (m, 3H), 2.24 (s, 1H), 2.15-2.02 (m, 2H), 2.02- 1.83 (m, 2H) |
| 123 | ((2- (((5S,8S,10aR)- 3-acetyl- 8-((4- cyanophenyl) (methyl)carba- moyl)-6- oxodecahydro- pyrrolo[1,2- a][1,5]diazocin- 5- yl)carbamoyl) benzo[b]thio- phen-5- yl)difluorometh- yl)phosphon- ic acid | 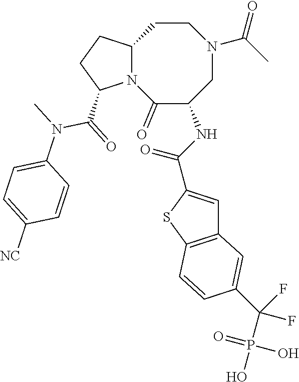 [SMILES: CC(=O)N1CC[C@H]2CC[C@@H](C(=O)N(C)c3ccc(C#N)cc3)N2C(=O)[C@@H](NC(=O)c2cc3cc(C(F)(F)P(=O)(O)O)ccc3s2)C1] | 674.0 [M + H]⁺ | ¹H NMR (500 MHz, DMSO-d₆) δ 8.71 (s, 1H), 8.35 (m, 1H), 8.08- 7.90 (m, 4H), 7.65 (s, 3H), 5.22 (s, 1H), 4.38 (m, 1H), 4.20 (s, 2H), 3.69 (s, 1H), 3.62 (s, 2H), 3.15 (s, 3H), 2.19 (m, 1H), 1.97 (s, 3H), 1.78 (m, 2H), 1.58 (s, 2H). |
| 124 | ((2- (((5S,8S,10aR)- 3-acetyl- 8-((3R,4S)-3- cyano-4- phenylpyrroli- dine-1- carbonyl)-6- oxodecahydro- pyrrolo[1,2- a][1,5]diazocin- 5- yl)carbamoyl) benzo[b]thio- phen-5- yl)difluorometh- yl)phosphon- ic acid | 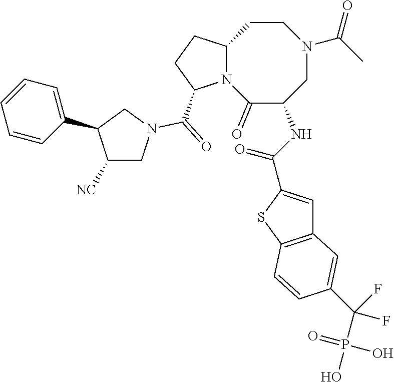 [SMILES: CC(=O)N1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(O)O)ccc3s2)C1] | 714.0 [M + H]⁺ | ¹H NMR (600 MHz, DMSO-d₆) δ 9.06-8.54 (m, 1H), 8.33-8.26 (m, 1H), 8.15-8.06 (m, 2H), 7.58 (m, 1H), 7.47- 7.28 (m, 5H), 4.92 (m, 1H), 4.63 (m, 1H), 4.38- 4.20 (m, 2H), 3.95 (m, 1H), 3.87-3.80 (m, 1H), 3.72-3.67 (m, 2H), 3.51 (m, 2H), 3.43 (m, 1H), 3.13-2.97 (m, 2H), 2.23 (s, 1H), 2.12 (m, 3H), 1.93 (s, 1H), 1.80 (m, 1H), 1.71 (m, 1H), 1.26-1.11 (m, 2H). |
| 125 | ((2- (((5S,8S,10aR)- 3-acetyl- 8-((4-(2- (dimethylami- no)ethoxy)phe- nyl)(methyl) carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 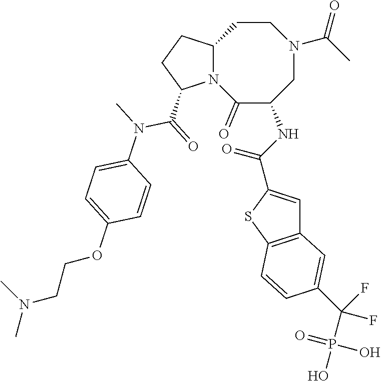 [SMILES: CC(=O)N1CC[C@H]2CC[C@@H](C(=O)N(C)c3ccc(OCCN(C)C)cc3)N2C(=O)[C@@H](NC(=O)c2cc3cc(C(F)(F)P(=O)(O)O)ccc3s2)C1] | 736.2 [M + H]⁺ | ¹H NMR (400 MHz, DMSO-d₆) δ 9.19 (m, 1H), 8.27-7.84 (m, 2H), 7.82 (s, 1H), 7.76- 7.38 (m, 2H), 7.15 (s, 2H), 6.42 (s, 1H), 4.95 (m, 1H), 4.43 (m, 1H), 4.30-4.14 (m, 2H), 3.99 (m, 1H), 3.82 (m, 2H), 3.72 (s, 1H), 3.51 (m, 2H), 3.05 (m, 3H), 2.93 (s, 1H), 2.66 (s, 6H), 2.17 (m, 3H), 2.04 (m, 2H), 1.82 (m, 2H), 1.65 (m, 1H). |
| 126 | ((2- (((5S,8S,10aR)- 3-acetyl- 6-oxo-8-((S)- 2-(pyridin-3- ylmethyl)pyr- rolidine-1- carbonyl)deca- hydropyrrolo [1,2- a][1,5]diazocin- 5- yl)carbamoyl) benzo[b]thio- phen-5- yl)difluorometh- yl)phosphon- ic acid | 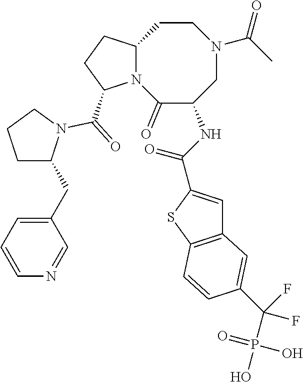 [SMILES: CC(=O)N1CC[C@H]2CC[C@@H](C(=O)N3CCC[C@H]3Cc3cccnc3)N2C(=O)[C@@H](NC(=O)c2cc3cc(C(F)(F)P(=O)(O)O)ccc3s2)C1] | 704.2 [M + H]⁺ | ¹H NMR (500 MHz, DMSO-d₆) δ 9.22-8.64 (m, 1H), 8.48 (m, 2H), 8.30 (s, 1H), 8.07 (m, 2H), 7.78 (m, 1H), 7.58 (m, 1H), 7.38 (m, 1H), 4.98 (m, 1H), 4.57 (m, 2H), 4.27 (s, 1H), 4.14 (s, 1H), 3.79 (m, 2H), 3.22 (s, 1H), 2.95 (m, 1H), 2.73 (s, 2H), 2.30 (s, 1H), 2.11 (m, 3H), 2.08-1.96 (m, 2H), 1.76 (s, 7H), 1.60 (s, 2H). |
| 127 | ((2- (((5S,8S,10aR)- 3-acetyl- 8-((6- chlorochroman- 3- yl)carbamoyl)- 6- oxodecahydro- pyrrolo[1,2- a][1,5]diazocin- 5- yl)carbamoyl) benzo[b]thio- phen-5- yl)difluorometh- yl)phosphon- ic acid | 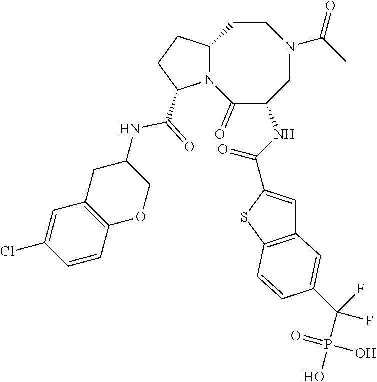 [SMILES: CC(=O)N1CC[C@H]2CC[C@@H](C(=O)NC3COc4ccc(Cl)cc4C3)N2C(=O)[C@@H](NC(=O)c2cc3cc(C(F)(F)P(=O)(O)O)ccc3s2)C1] | 725.0 [M + H]⁺ | ¹H NMR (500 MHz, DMSO-d₆) δ 8.66 (m, 1H), 8.30 (m, 1H), 8.18 (m, 1H), 8.12 (m, 1H), 8.08 (m, 1H), 7.57 (m, 1H), 7.23-7.14 (m, 1H), 7.10 (m, 1H), 6.86- 6.76 (m, 1H), 4.97- 4.80 (m, 1H), 4.34 (m, 1H), 4.29-4.20 (m, 1H), 4.08 (m, 2H), 3.84 (m, 2H), 3.68 (m, 1H), 3.62-3.54 (m, 1H), 3.42 (m, 1H), 3.03-2.96 (m, 1H), 2.68 (m, 1H), 2.14 (m, 3H), 2.08 (s, 1H), 1.97 (m, 1H), 1.89 (s, 1H), 1.81 (m, 1H), 1.76- 1.62 (m, 2H). |
| 128 | ((2- (((5S,8S,10aR)- 3-acetyl- 6-oxo-8-(3- (piperidine- 1- carbonyl)azeti- dine-1- carbonyl)deca- hydropyrrolo [1,2- a][1,5]diazocin- 5- yl)carbamoyl)- 1H-indol-5- yl)difluorometh- yl)phosphon- ic acid | 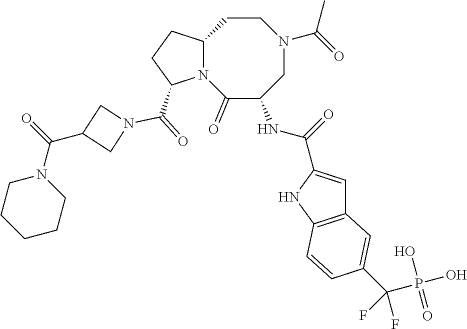 [SMILES: CC(=O)N1CC[C@H]2CC[C@@H](C(=O)N3CC(C(=O)N4CCCCC4)C3)N2C(=O)[C@@H](NC(=O)c2cc3cc(C(F)(F)P(=O)(O)O)ccc3[nH]2)C1] | 691.4 [M − H]⁻ | ¹H NMR (500 MHz, DMSO-d₆) δ 11.84 (m, 1H), 8.37 (m, 1H), 7.82 (s, 1H), 7.51-7.42 (m, 1H), 7.39-7.24 (m, 2H), 5.05-4.88 (m, 1H), 4.48 (m, 1H), 4.36- 4.19 (m, 3H), 3.99- 3.77 (m, 3H), 3.75-3.64 (m, 2H), 3.55 (m, 1H), 3.43 (m, 3H), 3.26 (m, 1H), 3.23-3.11 (m, 3H), 2.12 (m, 3H), 1.96 (m, 2H), 1.82-1.65 (m, 3H), 1.58-1.37 (m, 5H). |
| 129 | ((2- (((5S,8S,10aR)- 3-acetyl- 8-((R)-2- benzylpyrroli- dine-1- carbonyl)-6- oxodecahydro- pyrrolo[1,2- a][1,5]diazocin- 5- yl)carbamoyl)- 1H-indol-5- yl)difluorometh- yl)phosphon- ic acid | 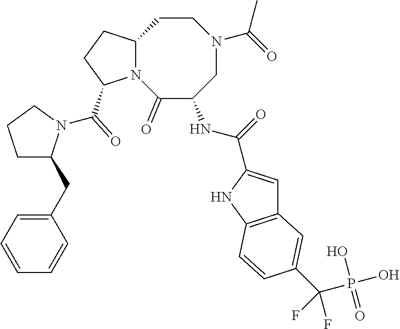 [SMILES: CC(=O)N1CC[C@H]2CC[C@@H](C(=O)N3CCC[C@@H]3Cc3ccccc3)N2C(=O)[C@@H](NC(=O)c2cc3cc(C(F)(F)P(=O)(O)O)ccc3[nH]2)C1] | 684.2 [M − H]⁻ | ¹H NMR (600 MHz, DMSO-d₆) δ 11.97- 11.71 (m, 1H), 8.47 (m, 7.4 Hz, 1H), 8.29-7.78 (m, 1H), 7.51-7.41 (m, 1H), 7.38-7.26 (m, 4H), 7.26-7.14 (m, 3H), 5.00-4.82 (m, 1H), 4.62-4.51 (m, 1H), 4.31-4.22 (m, 1H), 4.20-4.10 (m, 1H), 3.93-3.78 (m, 2H), 3.69-3.60 (m, 1H), 3.49-3.33 (m, 2H), 3.33-3.19 (m, 2H), 2.91 (m, 1H), 2.60- 2.54 (m, 1H), 2.25- 2.09 (m, 3H), 2.02 (m, 2H), 1.87-1.60 (m, 7H). |
| 130 | ((2- (((5S,8S,10aR)- 3-acetyl- 8-(2-(4- fluorobenzyl) pyrrolidine- 1-carbonyl)- 6- oxodecahydro- pyrrolo[1,2- a][1,5]diazocin- 5- yl)carbamoyl)- 1H-indol-5- yl)difluorometh- yl)phosphon- ic acid | 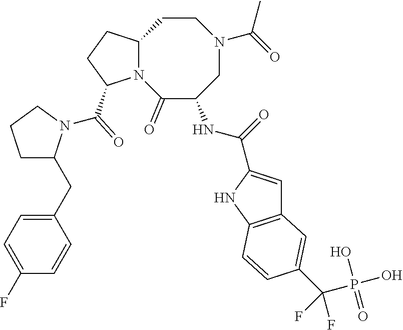 [SMILES: CC(=O)N1CC[C@H]2CC[C@@H](C(=O)N3CCCC3Cc3ccc(F)cc3)N2C(=O)[C@@H](NC(=O)c2cc3cc(C(F)(F)P(=O)(O)O)ccc3[nH]2)C1] | 702.2 [M − H]⁻ | ¹H NMR (600 MHz, DMSO-d₆) δ 12.03- 11.70 (m, 1H), 8.43- 8.22 (m, 1H), 7.81 (m, 1H), 7.53-7.44 (m, 1H), 7.39-7.23 (m, 3H), 7.23-7.06 (m, 3H), 5.12-4.92 (m, 1H), 4.62-4.50 (m, 1H), 4.26 (m, 1H), 4.20- 4.04 (m, 1H), 3.94- 3.56 (m, 4H), 3.53-3.19 (m, 4H), 2.99-2.84 (m, 1H), 2.23-2.09 (m, 3H), 2.02 (m, 1H), 1.95- 1.87 (m, 1H), 1.86- 1.62 (m, 7H). |
| 131 | ((2- (((5S,8S,10aR)- 3-acetyl- 8-(2-(4- methoxybenz- yl)pyrrolidine- 1- carbonyl)-6- oxodecahydro- pyrrolo[1,2- a][1,5]diazocin- 5- yl)carbamoyl)- 1H-indol-5- yl)difluorometh- yl)phosphon- ic acid | 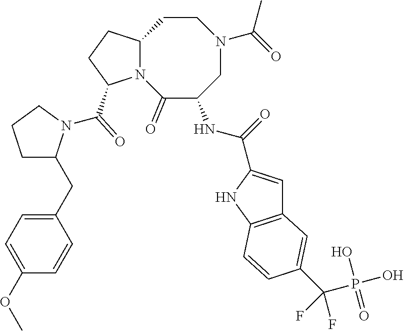 [SMILES: COc1ccc(CC2CCCN2C(=O)[C@@H]2CC[C@@H]3CCN(C(C)=O)C[C@H](NC(=O)c4cc5cc(C(F)(F)P(=O)(O)O)ccc5[nH]4)C(=O)N32)cc1] | 714.0 [M − H]⁻ | ¹H NMR (600 MHz, DMSO-d₆) δ 11.98- 11.70 (m, 1H), 8.80- 8.28 (m, 1H), 7.81 (m, 1H), 7.46 (m, 1H), 7.38- 7.32 (m, 1H), 7.28 (m, 1H), 7.22-7.05 (m, 2H), 6.87-6.79 (m, 2H), 5.11-4.81 (m, 1H), 4.67-4.52 (m, 1H), 4.28 (m, 1H), 4.16- 4.00 (m, 1H), 3.94- 3.73 (m, 2H), 3.69 (m, 3H), 3.67-3.54 (m, 2H), 3.50 (s, 1H), 3.46- 3.39 (m, 2H), 2.97-2.80 (m, 1H), 2.25-2.10 (m, 3H), 2.03 (m, 1H), 1.89- 1.55 (m, 7H). |
| 132 | ((2- (((5S,8S,10aR)- 3-acetyl- 8-(2-(3- chlorobenzyl) pyrrolidine- 1-carbonyl)- 6- oxodecahydro- pyrrolo[1,2- a][1,5]diazocin- 5- yl)carbamoyl)- 1H-indol-5- yl)difluorometh- yl)phosphon- ic acid | 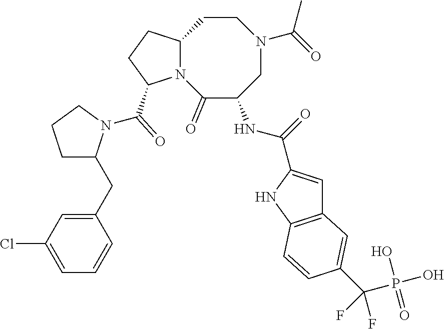 [SMILES: CC(=O)N1CC[C@H]2CC[C@@H](C(=O)N3CCCC3Cc3cccc(Cl)c3)N2C(=O)[C@@H](NC(=O)c2cc3cc(C(F)(F)P(=O)(O)O)ccc3[nH]2)C1] | 718.2 [M − H]⁻ | ¹H NMR (600 MHz, DMSO-d₆) δ 11.94- 11.66 (m, 1H), 8.36 (m, 1H), 7.79 (m, 1H), 7.48- 7.41 (m, 1H), 7.37- 7.21 (m, 5H), 7.16 (m, 1H), 5.09-4.82 (m, 1H), 4.57 (m, 1H), 4.38- 4.22 (m, 1H), 4.21- 4.02 (m, 1H), 3.98-3.76 (m, 2H), 3.64 (m, 2H), 3.42 (m, 2H), 3.04-2.84 (m, 1H), 2.74-2.61 (m, 1H), 2.24-2.09 (m, 3H), 2.07-1.94 (m, 2H), 1.87-1.61 (m, 8H). |
| 133 | ((2- (((5S,8S,10aR)- 3-acetyl- 8-(2-(2- fluorobenzyl) pyrrolidine- 1-carbonyl)- 6- oxodecahydro- pyrrolo[1,2- a][1,5]diazocin- 5- yl)carbamoyl)- 1H-indol-5- yl)difluorometh- yl)phosphon- ic acid | 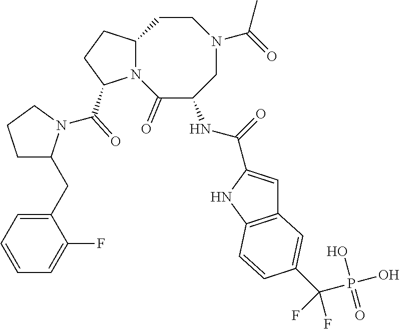 [SMILES: CC(=O)N1CC[C@H]2CC[C@@H](C(=O)N3CCCC3Cc3ccccc3F)N2C(=O)[C@@H](NC(=O)c2cc3cc(C(F)(F)P(=O)(O)O)ccc3[nH]2)C1] | 704.2 [M − H]⁻ | ¹H NMR (400 MHz, DMSO-d₆) δ 11.93- 11.74 (m, 1H), 7.97- 7.79 (m, 1H), 7.49 (m, 1H), 7.39-7.21 (m, 4H), 7.15 (m, 3H), 5.01 (m, 1H), 4.58 (m, 2H), 4.22 (m, 2H), 3.86-3.64 (m, 4H), 3.54-3.36 (m, 2H), 3.15 (m, 2H), 2.82 (m, 2H), 2.27-1.95 (m, 3H), 1.94-1.61 (m, 7H). |
| 134 | ((2- (((5S,8S,10aR)- 3-acetyl- 8- (cyclohexyl) (methyl)carba- moyl)-6- oxodecahydro- pyrrolo[1,2- a][1,5]diazocin- 5- yl)carbamoyl)- 1H-indol-5- yl)difluorometh- yl)phosphon- ic acid | 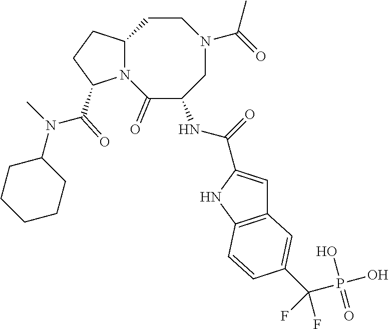 [SMILES: CC(=O)N1CC[C@H]2CC[C@@H](C(=O)N(C)C3CCCCC3)N2C(=O)[C@@H](NC(=O)c2cc3cc(C(F)(F)P(=O)(O)O)ccc3[nH]2)C1] | 636.2 [M − H]⁻ | ¹H NMR (500 MHz, DMSO-d₆) δ 11.84 (m, 1H), 8.63-8.26 (m, 1H), 7.81 (s, 1H), 7.46 (m, 1H), 7.36 (m, 1H), 7.32-7.24 (m, 1H), 4.97 (s, 1H), 4.84-4.71 (m, 1H), 4.29-4.18 (m, 2H), 3.90 (m, 2H), 3.40 (m, 2H), 3.24-3.15 (m, 1H), 2.78 (m, 3H), 2.26- 2.08 (m, 3H), 2.05- 1.93 (m, 1H), 1.87-1.64 (m, 5H), 1.56 (s, 3H), 1.50-1.22 (m, 5H), 1.07 (m, 1H). |
| 135 | ((2- (((5S,8S,10aR)- 3-acetyl- 8-(methyl(4- (trifluorometh- oxy)phenyl) carbamoyl)- 6- oxodecahydro- pyrrolo[1,2- a][1,5]diazocin- 5- yl)carbamoyl)- 1H-indol-5- yl)difluorometh- yl)phosphon- ic acid | 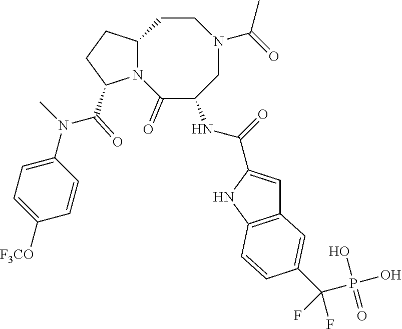 [SMILES: CC(=O)N1CC[C@H]2CC[C@@H](C(=O)N(C)c3ccc(OC(F)(F)F)cc3)N2C(=O)[C@@H](NC(=O)c2cc3cc(C(F)(F)P(=O)(O)O)ccc3[nH]2)C1] | 714.2 [M − H]⁻ | ¹H NMR (600 MHz, DMSO-d₆) δ 11.84 (m, 1H), 8.35 (m, 1H), 8.10 (m, 2H), 7.83 (s, 1H), 7.55 (m, 1H), 7.47 (m, 2H), 7.36 (m, 1H), 7.32 (m, 1H), 6.91 (m, 2H), 4.99-4.81 (m, 1H), 4.19 (s, 1H), 3.91-3.83 (m, 1H), 3.78-3.65 (m, 2H), 3.27 (m, 2H), 3.14 (m, 3H), 2.15 (m, 3H), 1.98-1.79 (m, 4H), 1.75- 1.60 (m, 2H). |
| 136 | ((2- (((5S,8S,10aR)- 3-acetyl- 8-((4-(tert- butyl)phenyl) (methyl)carba- moyl)-6- oxodecahydro- pyrrolo[1,2- a][1,5]diazocin- 5- yl)carbamoyl)- 1H-indol-5- yl)difluorometh- yl)phosphon- ic acid | 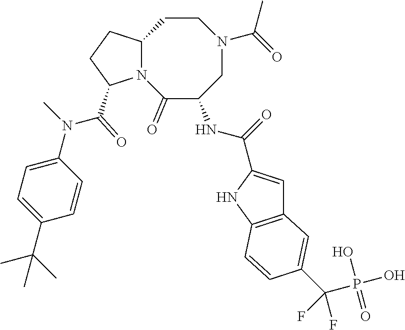 [SMILES: CC(=O)N1CC[C@H]2CC[C@@H](C(=O)N(C)c3ccc(C(C)(C)C)cc3)N2C(=O)[C@@H](NC(=O)c2cc3cc(C(F)(F)P(=O)(O)O)ccc3[nH]2)C1] | 686.0 [M − H]⁻ | ¹H NMR (500 MHz, DMSO-d₆) δ 11.83 (m, 1H), 8.49 (m, 1H), 7.83 (s, 1H), 7.47 (m, 3H), 7.36 (m, 1H), 7.29 (m, 3H), 4.86 (m, 1H), 4.21 (s, 1H), 4.14 (s, 1H), 3.86 (s, 2H), 3.75 (s, 2H), 3.13 (s, 3H), 2.15 (m, 3H), 1.95 (s, 1H), 1.84 (s, 3H), 1.65 (m, 2H), 1.30-1.22 (m, 9H). |
| 137 | ((2- (((5S,8S,10aR)- 3-acetyl- 8-((4-(2- methoxyetho- xy)phenyl) (methyl)carba- moyl)-6- oxodecahydro- pyrrolo[1,2- a][1,5]diazocin- 5- yl)carbamoyl)- 1H-indol-5- yl)difluorometh- yl)phosphon- ic acid | 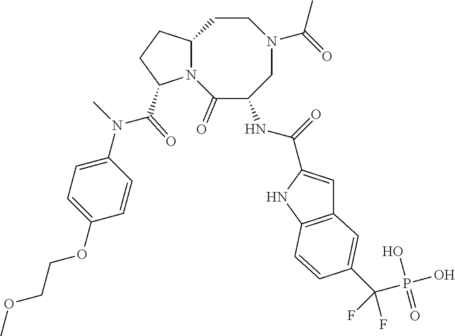 [SMILES: COCCOc1ccc(N(C)C(=O)[C@@H]2CC[C@@H]3CCN(C(C)=O)C[C@H](NC(=O)c4cc5cc(C(F)(F)P(=O)(O)O)ccc5[nH]4)C(=O)N32)cc1] | 704.0 [M − H]⁻ | ¹H NMR (500 MHz, DMSO-d₆) δ 11.94- 11.69 (m, 1H), 8.46 (m, 1H), 7.82 (s, 1H), 7.46 (m, 1H), 7.37 (m, 1H), 7.34-7.23 (m, 3H), 6.99 (m, 2H), 4.86 (m, 1H), 4.23 (s, 1H), 4.14 (s, 1H), 4.07 (m, 2H), 3.62 (m, 2H), 3.35-3.23 (m, 6H), 3.10 (m, 4H), 2.15 (m, 3H), 1.99-1.77 (m, 4H), 1.77-1.58 (m, 2H). |
| 138 | ((2- (((5S,8S,10aR)- 3-acetyl- 8-((3R,4S or 3S,4R)-3- cyano-4- phenylpyrroli- dine-1- carbonyl)-6- oxodecahydro- pyrrolo[1,2- a][1,5]diazocin- 5- yl)carbamoyl)- 1H-indol-5- yl)difluorometh- yl)phosphon- ic acid | 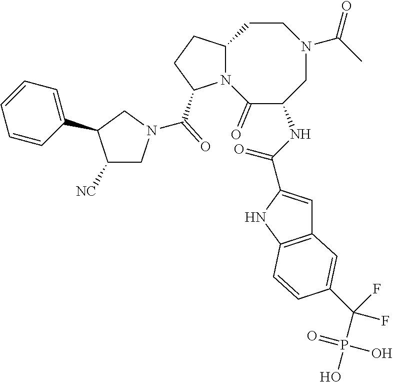 [SMILES: CC(=O)N1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(O)O)ccc3[nH]2)C1] | 695.4 [M − H]⁺ | ¹H NMR (600 MHz, DMSO-d₆) δ 11.84 (m, 1H), 8.49 (m, 1H), 7.83 (m, 1H), 7.52-7.25 (m, 8H), 5.04-4.86 (m, 1H), 4.58 (m, 1H), 4.26 (m, 1H), 4.20 (m, 1H), 4.09 (m, 1H), 4.02-3.80 (m, 4H), 3.66-3.55 (m, 2H), 3.52-3.38 (m, 1H), 3.34-3.24 (m, 2H), 2.21-2.08 (m, 3H), 1.96 (m, 2H), 1.89- 1.68 (m, 3H). |
| | | or | | |
| | | 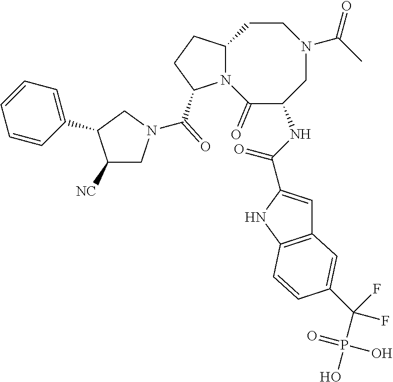 [SMILES: CC(=O)N1CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3[nH]2)C1] | | |
| | | SFC peak 1 was used for | | |
| | | biological testing | | |
| 139 | ((2- (((5S,8S,10aR)- 3-acetyl- 8-((3S,4R or 3R,4S)-3- cyano-4- phenylpyrroli- dine-1- carbonyl)-6- oxodecahydro- pyrrolo[1,2- a][1,5]diazocin- 5- yl)carbamoyl)- 1H-indol-5- yl)difluorometh- yl)phosphon- ic acid | 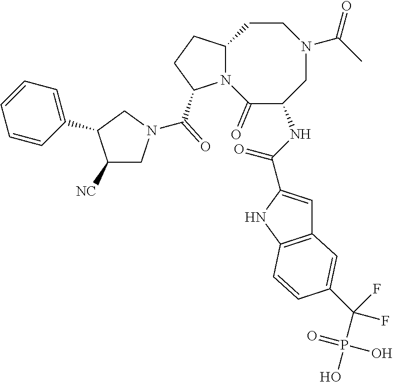 [SMILES: CC(=O)N1CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3[nH]2)C1] | 695.2 [M − H]⁻ | ¹H NMR (600 MHz, DMSO-d₆) δ 11.93- 11.70 (m, 1H), 8.69- 8.28 (m, 1H), 7.82 (m, 1H), 7.50-7.25 (m, 8H), 4.95 (m, 1H), 4.62 (m, 1H), 4.41-4.24 (m, 2H), 4.02-3.85 (m, 2H), 3.75 (m, 4H), 3.57- 3.40 (m, 2H), 3.24 (s, 2H), 2.34-2.26 (m, 1H), 2.22-2.07 (m, 3H), 1.97 (m, 1H), 1.88- 1.65 (m, 3H). |
| | | or | | |
| | | 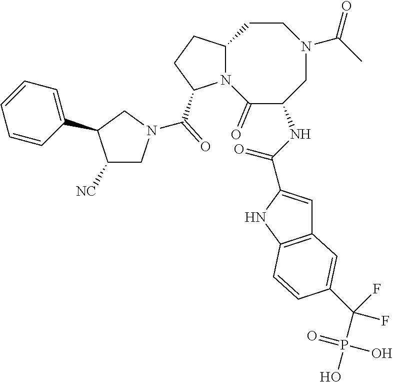 [SMILES: CC(=O)N1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(O)O)ccc3[nH]2)C1] | | |
| | | SFC peak 2 was used for | | |
| | | biological testing | | |
| 140 | ((2- (((5S,8S,10aR)- 3-acetyl- 6-oxo-8-((2- oxo-1- phenyl-1,2- dihydropyridin- 4- yl)carbamoyl) decahydropyr- rolo[1,2- a][1,5]diazocin- 5- yl)carbamoyl)- 1H-indol-5- yl)difluorometh- yl)phosphon- ic acid | 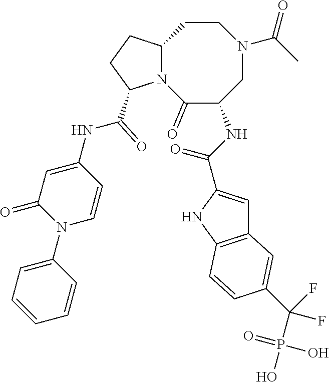 [SMILES: CC(=O)N1CC[C@H]2CC[C@@H](C(=O)Nc3ccn(-c4ccccc4)c(=O)c3)N2C(=O)[C@@H](NC(=O)c2cc3cc(C(F)(F)P(=O)(O)O)ccc3[nH]2)C1] | 711.2 [M + H]⁺ | ¹H NMR (600 MHz, DMSO-d₆) δ 11.83 (m, 1H), 10.39 (m, 1H), 8.56 (m, 1H), 7.82 (s, 1H), 7.57 (m, 1H), 7.48 (m, 3H), 7.41 (m, 1H), 7.35 (m, 3H), 7.29 (m, 1H), 6.84-6.79 (m, 1H), 6.45 (m, 1H), 5.10-4.93 (m, 1H), 4.46 (m, 1H), 4.39- 4.29 (m, 1H), 3.85- 3.79 (m, 1H), 3.66 (m, 2H), 3.45-3.39 (m, 2H), 2.29-2.24 (m, 1H), 2.17-2.08 (m, 3H), 1.92 (m, 2H), 1.83- 1.73 (m, 2H). |

### Prepared According to General Scheme 2: Phosphonic Acids

The following compounds in Table 15 were prepared according to the representative procedure described above for the synthesis of ((2-(((5S,8S,10aR)-3-acetyl-8-((4-(2-cyanopropan-2-yl)phenyl)(methyl)carbamoyl)-6-oxodecahydropyrrolo[1,2-a][1,5]diazocin-5-yl)carbamoyl)benzo[b]thiophen-5-yl)difluoromethyl)phosphonic acid (2) utilizing the appropriate starting materials and modifications.

TABLE 15

| Comp- | | | | |
|---|---|---|---|---|
| ound | Name | Structure | LCMS | NMR |
| 141 | ((2- (((5S,8S,10a R)-3-acetyl- 8-((rel- trans)-3- cyano-4-(1H- pyrazol-4- yl)pyrrolidine- 1- carbonyl)-6- oxodeca- hydropyrrolo[1,2- a][1,5] diazocin-5- yl)carbamoyl) benzo[b]thio- phen-5-yl) difluoromethyl) phosphonic acid | 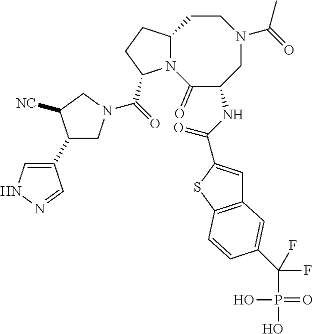 | 704 M + H]⁺ | ¹H NMR (400 MHz, DMSO-d₆) δ 9.06- 8.59 (m, 2H), 8.36- 8.27 (m, 1H), 8.17- 8.05 (m, 2H), 7.74- 7.53 (m, 3H), 5.05- 4.88 (m, 1H), 4.64- 4.55 (m, 1H), 4.33- 4.23 (m, 2H), 4.14- 4.08 (m, 1H), 4.04- 3.97 (m, 2H), 3.90- 3.86 (m, 2H), 3.40- 3.36 (m, 2H), 3.32- 3.24 (m, 2H), 2.19- 1.97 (m, 6H), 1.85- 1.72 (m, 3H). |
| 142 | ((2- (((5S,8S,10a R)-3-acetyl- 8-((rel- trans)-3- cyano-4-(3,5- difluorophenyl) pyrrolidine- 1- carbonyl)-6- oxodeca- hydropyrrolo[1,2- a][1,5] diazocin-5- yl)carbamoyl) benzo[b]thio- phen-5-yl) difluoromethyl) phosphonic acid | 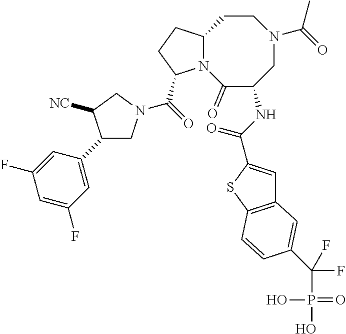 | 750 [M + H]⁺ | ¹H NMR (400 MHz, DMSO-d₆) δ 9.07- 8.49 (m, 1H), 8.45- 8.22 (m, 1H), 8.19- 8.02 (m, 2H), 7.60 (d, J = 8.4 Hz, 1H), 7.40- 7.01 (m, 3H), 5.00- 4.82 (m, 1H), 4.65- 4.55 (m, 1H), 4.33- 4.27 (m, 1H), 4.22- 4.10 (m, 1H), 3.99- 3.84 (m, 3H), 3.77- 3.58 (m, 3H), 3.57- 3.45 (m, 1H), 3.42- 3.21 (m, 2H), 2.42- 2.24 (m, 1H), 2.20- 2.08 (m, 3H), 2.07- 1.88 (m, 2H), 1.88- 1.59 (m, 3H). |
| 143 | ((2- (((5S,8S,10a R)-3-acetyl- 8-((rel- trans)-3,4- diphenyl- pyrrolidine-1- carbonyl)-6- oxodeca- hydropyrrolo[1,2- a][1,5]diazocin- 5- yl)carbamoyl) benzo[b]thio- phen-5-yl) difluoromethyl) phosphonic acid | 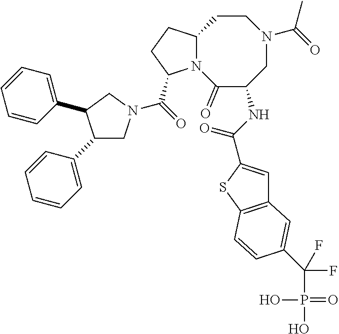 | 765 [M + H]⁺ | ¹H NMR (400 MHz, DMSO-d₆) δ 9.05- 8.60 (m, 1H), 8.33- 8.29 (m, 1H), 8.14- 8.03 (m, 2H), 7.68- 7.62 (m, 1H), 7.34- 7.22 (m, 8H), 7.19- 7.12 (m, 2H), 5.04- 4.83 (m, 1H), 4.73- 4.58 (m, 1H), 4.41- 4.22 (m, 2H), 4.19- 4.06 (m, 1H), 4.04- 3.98 (m, 1H), 3.94- 3.85 (m, 1H), 3.77- 3.69 (m, 2H), 3.62- 3.45 (m, 2H), 3.37- 3.28 (m, 2H), 2.43- 2.29 (m, 1H), 2.22- 2.09 (m, 3H), 2.04- 1.90 (m, 2H), 1.90- 1.67 (m, 3H). |
| 144 | ((2- (((5S,8S,10a R)-3-acetyl- 8-((rel- trans)-3- cyano-4-(2- methoxyphenyl) pyrrolidine- 1-carbonyl)-6- oxodeca- hydropyrrolo[1,2- a][1,5]diazocin- 5-yl) carbamoyl) benzo[b]thio- phen-5- yl)difluoromethyl) phosphonic acid | 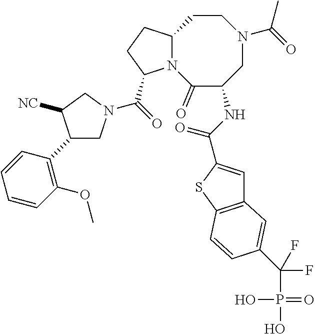 | 373 [M/2 + H]⁺ | ¹H NMR (400 MHz, DMSO-d₆) δ 9.11- 8.60 (m, 1H), 8.38- 8.29 (m, 1H), 8.19- 8.06 (m, 2H), 7.64- 7.56 (m, 1H), 7.41- 7.18 (m, 2H), 7.11- 6.90 (m, 2H), 5.10- 4.95 (m, 1H), 4.94- 4.74 (m, 1H), 4.66- 4.57 (m, 1H), 4.37- 4.23 (m, 3H), 4.02- 3.98 (m, 2H), 3.95- 3.91 (m, 2H), 3.86- 3.84 (m, 3H), 3.73- 3.70 (m, 1H), 3.57- 3.50 (m, 1H), 3.38- 3.30 (m, 1H), 2.37- 2.24 (m, 1H), 2.20- 2.09 (m, 3H), 2.02- 1.66 (m, 5H). |
| 145 | ((2- (((5S,8S,10a R)-3-acetyl- 8-((rel- trans)-3- cyano-4-(2- oxo-1,2- dihydropyridin- 4- yl)pyrrolidine- 1- carbonyl)-6- oxodeca- hydropyrrolo[1,2- a][1,5]diazocin- 5- yl)carbamoyl) benzo[b]thio- phen-5-yl) difluoromethyl) | 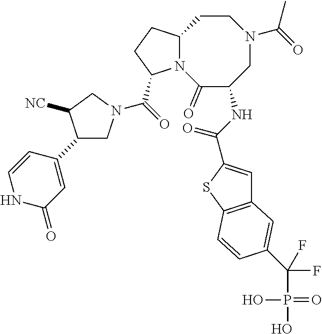 | 731 [M + H]⁺ | ¹H NMR (400 MHz, DMSO-d₆) δ 9.12- 8.58 (m, 1H), 8.38- 8.26 (m, 1H), 8.20- 8.03 (m, 2H), 7.60- (d, J = 8.0 Hz, 1H), 7.45- 7.28 (m, 1H), 6.46- 6.12 (m, 2H), 5.05- 4.85 (m, 1H), 4.74- 4.59 (m, 1H), 4.38- 4.23 (m, 2H), 4.11- 3.88 (m, 3H), 3.81- 3.58 (m, 4H), 3.45- 3.19 (m, 2H), 2.44- 2.24 (m, 1H), 2.22- 2.09 (m, 3H), 2.07- 1.65 (m, 5H). |
| | phosphonic | | | |
| | acid | | | |
| 146 | ((2- (((5S,8S,10a R)-3-acetyl- 8-((rel- trans)-3- cyano-4-(6- oxo-1,6- dihydropyridin- 3- yl)pyrrolidine- 1- carbonyl)-6- oxodeca- hydropyrrolo [1,2- a][1,5]diazocin- 5- yl)carbamoyl) benzo[b]thio- phen-5-yl) | 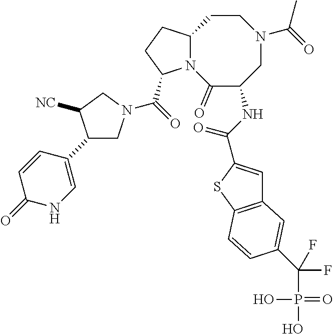 | 731 [M + H]⁺ | ¹H NMR (400 MHz, DMSO-d₆) δ 11.71 (s, 1H), 9.15-8.52 (m, 1H), 8.41-8.23 (m, 1H), 8.17-7.98 (m, 2H), 7.67-6.90 (m, 3H), 6.46-6.27 (m, 1H), 5.03-4.84 (m, 1H), 4.70-4.56 (m, 1H), 4.32-4.14 (m, 2H), 3.98-3.82 (m, 3H), 3.74-3.54 (m, 4H), 3.42-3.22 (m, 2H), 2.45-2.23 (m, 1H), 2.21-2.08 (m, 3H), 2.10-1.60 (m, 5H). |
| | difluoromethyl) | | | |
| | phosphonic | | | |
| | acid | | | |
| 147 | ((2- (((5S,8S,10a R)-3-acetyl- 8-((rel- trans)-3- cyano-4-(6- methoxypyridin- 3- yl)pyrrolidine- 1- carbonyl)-6- oxodeca- hydropyrrolo[1,2- a][1,5]diazocin- 5- yl)carbamoyl) benzo[b]thio- phen-5-yl) difluoromethyl) phosphonic acid | 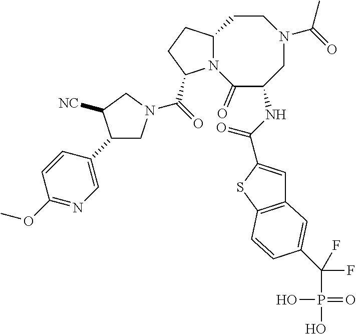 | 745 [M + H]⁺ | ¹H NMR (400 MHz, DMSO-d₆) δ 9.17- 8.55 (m, 1H), 8.37- 8.29 (m, 1H), 8.27- 8.04 (m, 3H), 7.88- 7.65 (m, 1H), 7.60 (d, J = 8.1 Hz, 1H), 6.92- 6.78 (m, 1H), 5.03- 4.84 (m, 1H), 4.69- 4.55 (m, 1H), 4.37- 4.19 (m, 2H), 4.13- 3.89 (m, 3H), 3.87- 3.82 (m, 3H), 3.75- 3.50 (m, 4H), 3.46- 3.25 (m, 2H), 2.44- 2.23 (m, 1H), 2.21- 2.08 (m, 3H), 2.08- 1.64 (m, 5H). |
| 148 | ((2- (((5S,8S,10a R)-3-acetyl- 8-((rel- trans)-3- cyano-4-(2- methoxypyridin- 4- yl)pyrrolidine- 1- carbonyl)-6- oxodeca- hydropyrrolo[1,2- a][1,5]diazocin- 5- yl)carbamoyl) benzo[b]thio- phen-5-yl) difluoromethyl) phosphonic acid | 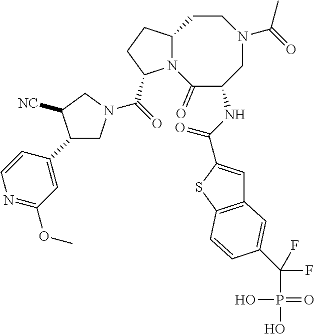 | 745 [M + H]⁺ | ¹H NMR (400 MHz, DMSO-d₆) δ 9.10- 8.55 (m, 1H), 8.38- 8.04 (m, 4H), 7.67- 7.53 (m, 1H), 7.13- 6.70 (m, 2H), 5.08- 4.82 (m, 1H), 4.75- 4.45 (m, 2H), 4.40- 4.11 (m, 4H), 4.01- 3.94 (m, 3H), 3.85- 3.84 (m, 3H), 3.72- 3.71 (m, 1H), 3.42- 3.31 (m, 2H), 2.38- 2.26 (m, 1H), 2.20- 2.00 (m, 4H), 1.98- 1.71 (m, 4H). |
| 149 | ((2- (((5S,8S,10a R)-3-acetyl- 8-((rel- trans)-3- cyano-4-(3- methoxyphenyl) pyrrolidine- 1- carbonyl)-6- oxodeca- hydropyrrolo[1,2- | 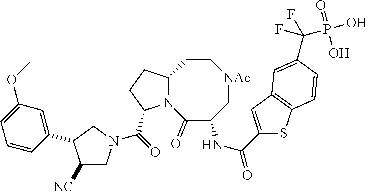 | 744.3 [M + H]⁺ | ¹H NMR (400 MHz, DMSO-d₆) δ 9.09- 8.56 (m, 1H), 8.38- 8.24 (m, 1H), 8.21- 8.03 (m, 2H), 7.60 (d, J = 8.4 Hz, 1H), 7.35- 7.26 (m, 1H), 7.09- 6.85 (m, 3H), 5.04- 4.80 (m, 1H), 4.67- 4.52 (m, 1H), 4.36- 4.18 (m, 2H), 4.01- 3.84 (m, 3H), 3.79- |
| | a][1,5]diazocin- | | | 3.73 (m, 4H), 3.64- |
| | 5- | | | 3.50 (m, 3H), 3.37- |
| | yl)carbamoyl) | | | 3.29 (m, 2H), 2.38- |
| | benzo[b]thio- | | | 2.26 (m, 1H), 2.19- |
| | phen-5-yl) | | | 1.94 (m, 5H), 1.86- |
| | difluoromethyl) | | | 1.67 (m, 3H). |
| | phosphonic | | | |
| | acid | | | |
| 150 | ((2- (((5S,8S,10a R)-3-acetyl- 8-((rel- trans)-3- cyano-4- (pyridin-3- yl) pyrrolidine-1- carbonyl)-6- oxodeca- hydropyrrolo[1,2- | 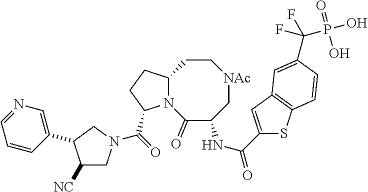 | 715.3 [M + H]⁺ | ¹H NMR (400 MHz, METHANOL-d₄) δ 8.89-8.56 (m, 2H), 8.57-8.28 (m, 1H), 8.27-8.01 (m, 2H), 7.98-7.79 (m, 2H), 7.78-7.59 (m, 1H), 5.21-5.04 (m, 1H), 4.76-4.65 (m, 1H), 4.47-4.31 (m, 1H), 4.18-4.04 (m, 3H), 4.03-3.82 (m, 4H), |
| | a][1,5]diazocin- | | | 3.72-3.54 (m, 3H), |
| | 5- | | | 2.29-2.26 (m, 1H), |
| | yl)carbamoyl) | | | 1.99-1.85 (m, 2H), |
| | benzo[b]thio- | | | 1.30-1.25 (m, 4H), |
| | phen-5-yl) | | | 0.85-0.76 (m, 2H). |
| | difluoromethyl) | | | |
| | phosphonic | | | |
| | acid | | | |
| 151 | ((2- (((5S,8S,10a R)-3-acetyl- 8-((rel- trans)-3- cyano-4- cyclohexyl- pyrrolidine-1- carbonyl)-6- | 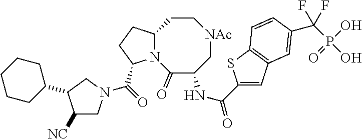 | 720.3 [M + H]⁺ | ¹H NMR (400 MHz, DMSO) δ 9.14-8.56 (m, 1H), 8.39-8.26 (m, 1H), 8.21-8.03 (m, 2H), 7.60 (d, J = 8.3 Hz, 1H), 5.02- 4.82 (m, 1H), 4.65- 4.52 (m, 1H), 4.33- 4.17 (m, 2H), 4.09- |
| | oxodeca- | | | 3.98 (m, 1H), 3.86- |
| | hydropyrrolo[1,2- | | | 3.66 (m, 5H), 3.21- |
| | a][1,5] | | | 2.89 (m, 2H), 2.19- |
| | diazocin-5- | | | 1.13 (m, 21H). |
| | yl)carbamoyl) | | | |
| | benzo[b]thio- | | | |
| | phen-5-yl) | | | |
| | difluoromethyl) | | | |
| | phosphonic | | | |
| | acid | | | |
| 152 | ((2- (((5S,8S,10a R)-3-acetyl- 8-((rel- trans)-4- cyano-3- methyl-3- phenyl- pyrrolidine-1- carbonyl)-6- oxodeca- | 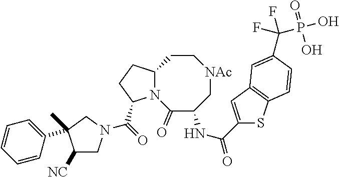 | 728.3 [M + H]⁺ | ¹H NMR (400 MHz, DMSO-d₆) δ 9.18- 8.58 (m, 1H), 8.38- 8.26 (m, 1H), 8.20- 8.06 (m, 2H), 7.65- 7.55 (m, 1H), 7.53- 7.24 (m, 5H), 5.07- 5.00 (m, 1H), 4.66- 4.60 (m, 1H), 4.27- 4.15 (m, 1H), 4.07- 3.99 (m, 1H), 3.93- |
| | hydropyrrolo[1,2- | | | 3.53 (m, 6H), 3.47- |
| | a][1,5]diazocin- | | | 3.29 (m, 2H), 2.46- |
| | 5- | | | 2.26 (m, 1H), 2.21- |
| | yl)carbamoyl) | | | 2.08 (m, 3H), 2.06- |
| | benzo[b]thio- | | | 1.87 (m, 2H), 1.85- |
| | phen-5-yl) | | | 1.68 (m, 3H), 1.55- |
| | difluoromethyl) | | | 1.41 (m, 3H). |
| | phosphonic | | | |
| | acid | | | |
| 153 | ((2- (((5S,8S,10a R)-3-acetyl- 8-((rel- trans)-3- cyano-5- phenyl- piperidine-1- carbonyl)-6- oxodeca- hydropyrrolo[1,2- a][1,5] | 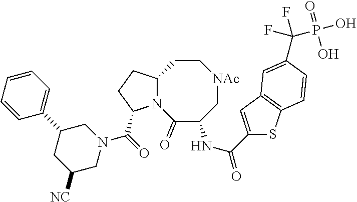 | 728.3 [M + H]⁺ | ¹H NMR (400 MHz, DMSO-d₆) δ 9.19 8.45 (m, 1H), 8.39- 8.24 (m, 1H), 8.19- 8.04 (m, 2H), 7.59 (d, J = 7.6 Hz, 1H), 7.43- 7.20 (m, 5H), 5.12- 4.83 (m, 2H), 4.76- 4.43 (m, 2H), 4.14- 3.67 (m, 5H), 3.46- 3.14 (m, 4H), 2.94- 2.61 (m, 2H), 2.44- |
| | diazocin-5- | | | 2.19 (m, 2H), 2.16- |
| | yl)carbamoyl) | | | 2.09 (m, 3H), 2.03- |
| | benzo[b]thio- | | | 1.56 (m, 4H). |
| | phen-5-yl) | | | |
| | difluoromethyl) | | | |
| | phosphonic | | | |
| | acid | | | |
| 154 | ((2- (((5S,8S,10a R)-3-acetyl- 8-((rel- trans)-3- cyano-4- phenyl- piperidine-1- carbonyl)-6- oxodeca- hydropyrrolo[1,2- | 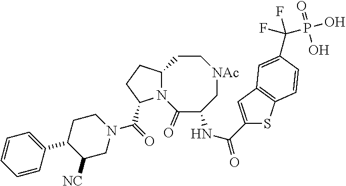 | 728.3 [M + H]⁺ | ¹H NMR (400 MHz, DMSO-d₆) δ 9.20- 8.55 (m, 1H), 8.40- 8.27 (m, 1H), 8.22- 8.05 (m, 2H), 7.60 (d, J = 8.1 Hz, 1H), 7.43- 7.19 (m, 5H), 5.19- 4.64 (m, 3H), 4.63- 4.06 (m, 3H), 4.05- 3.61 (m, 3H), 3.56- 3.35 (m, 2H), 3.12- |
| | a][1,5]diazocin- | | | 2.94 (m, 2H), 2.41- |
| | 5- | | | 2.23 (m, 1H), 2.23- |
| | yl)carbamoyl) | | | 2.09 (m, 3H), 2.08- |
| | benzo[b]thio- | | | 1.47 (m, 7H). |
| | phen-5-yl) | | | |
| | difluoromethyl) | | | |
| | phosphonic | | | |
| | acid | | | |
| 155 | ((2- (((5S,8S,10a R)-3-acetyl- 8-(methyl(4- (2- phenylpropan- 2- yl)phenyl) carbamoyl)-6- oxodeca- hydropyrrolo[1,2- a][1,5]diazocin-5- yl)carbamoyl) benzo[b]thio- phen-5-yl) difluoromethyl) | 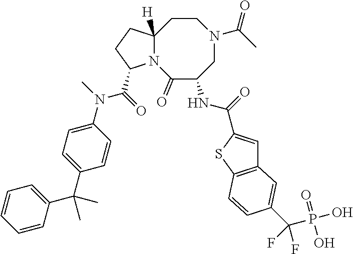 [SMILES: CC(=O)N1CC[C@H]2CC[C@@H](C(=O)N(C)c3ccc(C(C)(C)c4ccccc4)cc3)N2C(=O)[C@@H](NC(=O)c2cc3cc(C(F)(F)P(=O)(O)O)ccc3s2)C1] | 767.4 [M + H]⁺ | ¹H NMR (400 MHz, DMSO-d₆) δ 9.01- 8.59 (m, 1H), 8.39- 8.30 (m, 1H), 8.19- 8.07 (m, 2H), 7.66- 7.57 (m, 1H), 7.35- 7.19 (m, 8H), 7.19- 7.11 (m, 1H), 5.00- 4.75 (m, 1H), 4.28- 3.96 (m, 3H), 3.91- 3.64 (m, 3H), 3.36- 3.28 (m, 1H), 3.20- 3.07 (m, 3H), 2.20- 2.07 (m, 3H), 2.00- 1.71 (m, 5H), 1.65- 1.60 (m, 6H). |
| | phosphonic | | | |
| | acid | | | |
| 156 | ((2- (((5S,8S,10a R)-3-acetyl- 8-((4-(1- hydroxy-2- methylpropan- 2- yl)phenyl) (methyl) carbamoyl)-6- oxodeca- hydropyrrolo[1,2- a][1,5]diazocin- 5- yl)carbamoyl) | 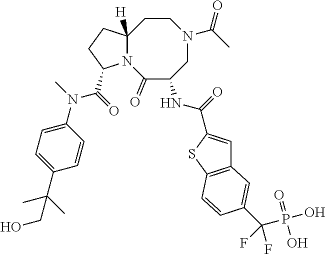 [SMILES: CC(=O)N1CC[C@H]2CC[C@@H](C(=O)N(C)c3ccc(C(C)(C)CO)cc3)N2C(=O)[C@@H](NC(=O)c2cc3cc(C(F)(F)P(=O)(O)O)ccc3s2)C1] | 721.4 [M + H]⁺ | ¹H NMR (400 MHz, DMSO-d₆) δ 9.01- 8.59 (m, 1H), 8.39- 8.30 (m, 1H), 8.19- 8.06 (m, 2H), 7.63- 7.56 (m, 1H), 7.51- 7.37 (m, 2H), 7.35- 7.16 (m, 2H), 4.96- 4.75 (m, 1H), 4.29- 4.14 (m, 2H), 3.92- 3.76 (m, 2H), 3.41- 3.31 (m, 4H), 3.16- 3.11 (m, 3H), 2.22- 2.03 (m, 4H), 1.93- 1.65 (m, 5H), 1.20 (s, |
| | benzo[b]thio- | | | 6H). |
| | phen-5-yl) | | | |
| | difluoromethyl) | | | |
| | phosphonic | | | |
| | acid | | | |
| 157 | ((2- (((5S,8S,10a R)-3-acetyl- 8-((2-methyl- 2H-indazol- 6- yl)carbamoyl)- 6- oxodeca- hydropyrrolo[1,2- a][1,5]diazocin- 5- yl)carbamoyl) benzo[b]thio- phen-5-yl) difluoromethyl) | 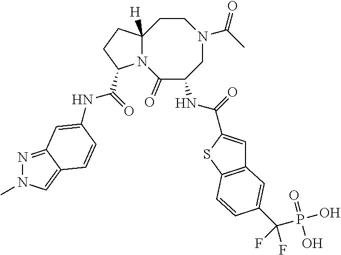 [SMILES: CC(=O)N1CC[C@H]2CC[C@@H](C(=O)Nc3ccc4cn(C)nc4c3)N2C(=O)[C@@H](NC(=O)c2cc3cc(C(F)(F)P(=O)(O)O)ccc3s2)C1] | 689.3 [M + H]⁺ | ¹H NMR (400 MHz, DMSO-d₆) δ 10.38- 10.25 (m, 1H), 9.08- 8.67 (m, 1H), 8.37- 8.31 (m, 1H), 8.20- 8.11 (m, 2H), 8.10- 8.05 (m, 1H), 8.00- 7.95 (m, 1H), 7.62- 7.45 (m, 3H), 5.06- 4.79 (m, 1H), 4.62- 4.56 (m, 1H), 4.03- 4.01 (m, 3H), 3.91- 3.68 (m, 3H), 3.57- 3.37 (m, 2H), 2.36- 2.27 (m, 1H), 2.20- 2.12 (m, 3H), 2.11- |
| | phosphonic | | | 1.71 (m, 5H). |
| | acid | | | |
| 158 | ((2- (((5S,8S,10a R)-3-acetyl- 8- (isoquinolin- 6- ylcarbamoyl)- 6-oxodeca- hydropyrrolo[1,2- a][1,5] diazocin- 5- yl)carbamoyl) benzo[b]thio- phen-5-yl) difluoromethyl) | 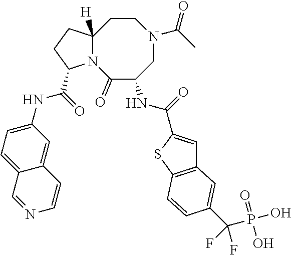 [SMILES: CC(=O)N1CC[C@H]2CC[C@@H](C(=O)Nc3ccc4cnccc4c3)N2C(=O)[C@@H](NC(=O)c2cc3cc(C(F)(F)P(=O)(O)O)ccc3s2)C1] | 686 [M + H]⁺ | ¹H NMR (400 MHz, DMSO-d₆) δ 11.12- 11.03 (m, 1H), 9.67- 9.59 (m, 1H), 9.05- 8.67 (m, 2H), 8.62- 8.52 (m, 1H), 8.45- 8.38 (m, 1H), 8.36- 8.27 (m, 2H), 8.18- 8.11 (m, 1H), 8.09- 8.04 (m, 1H), 7.96- 7.91 (m, 1H), 7.63- 7.55 (m, 1H), 5.08- 4.88 (m, 1H), 4.66- 4.60 (m, 1H), 4.42- 4.35 (m, 1H), 3.98- 3.91 (m, 1H), 3.83- |
| | phosphonic | | | 3.76 (m, 1H), 3.68- |
| | acid | | | 3.59 (m, 1H), 3.46- |
| | | | | 3.40 (m, 1H), 2.39- |
| | | | | 2.29 (m, 1H), 2.19- |
| | | | | 2.11 (m, 4H), 2.04- |
| | | | | 1.76 (m, 4H). (TFA |
| | | | | salt) |
| 159 | ((2- (((5S,8S,10a R)-3-acetyl- 8-(methyl(4- (tert- pentyl)phenyl) carbamoyl) -6- oxodeca- hydropyrrolo[1,2- a][1,5]diazocin- 5- yl)carbamoyl) benzo[b]thio- phen-5-yl) difluoromethyl) | 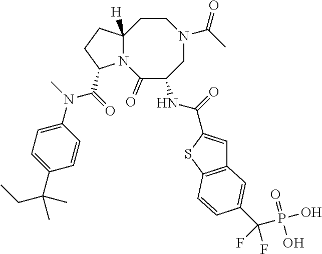 [SMILES: CCC(C)(C)c1ccc(N(C)C(=O)[C@@H]2CC[C@@H]3CCN(C(C)=O)C[C@H](NC(=O)c4cc5cc(C(F)(F)P(=O)(O)O)ccc5s4)C(=O)N32)cc1] | 719 [M + H]⁺ | ¹H NMR (400 MHz, DMSO-d₆) δ 8.99- 8.59 (m, 1H), 8.39- 8.30 (m, 1H), 8.18- 8.08 (m, 2H), 7.63- 7.58 (m, 1H), 7.46- 7.26 (m, 4H), 4.98- 4.72 (m, 1H), 4.30- 4.12 (m, 2H), 3.99- 3.77 (m, 2H), 3.39- 3.30 (m, 2H), 3.18- 3.10 (m, 3H), 2.21- 2.03 (m, 3H), 1.96- 1.55 (m, 8H), 1.31- 1.15 (m, 6H), 0.68- 0.54 (m, 3H). |
| | phosphonic | | | |
| | acid | | | |
| 160 | ((2- (((5S,8S,10a R)-3-acetyl- 8-((4-(tert- butyl)-3- chlorophenyl) (methyl) carbamoyl)-6- oxodeca- hydropyrrolo[1,2- a][1,5]diazocin- 5- yl)carbamoyl )benzo[b]thio- phen-5-yl) | 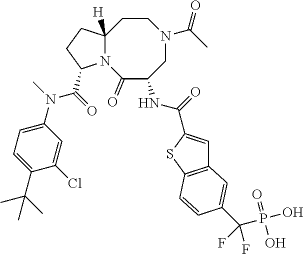 [SMILES: CC(=O)N1CC[C@H]2CC[C@@H](C(=O)N(C)c3ccc(C(C)(C)C)c(Cl)c3)N2C(=O)[C@@H](NC(=O)c2cc3cc(C(F)(F)P(=O)(O)O)ccc3s2)C1] | 739 [M + H]⁺ | ¹H NMR (400 MHz, DMSO-d₆) δ 9.07- 8.64 (m, 1H), 8.39- 8.32 (m, 1H), 8.18- 8.08 (m, 2H), 7.65- 7.27 (m, 4H), 4.98- 4.78 (m, 1H), 4.29- 4.12 (m, 2H), 3.97- 3.66 (m, 3H), 3.40- 3.31 (m, 1H), 3.22- 3.03 (m, 3H), 2.22- 2.07 (m, 3H), 1.99- 1.60 (m, 6H), 1.53- 1.37 (m, 9H). |
| | difluoromethyl) | | | |
| | phosphonic | | | |
| | acid | | | |
| 161 | ((2- (((5S,8S,10a R)-3-acetyl- 8-((1-methyl- 1H-indazol- 5- yl)carbamoyl)- 6- oxodeca- hydropyrrolo[1,2- a][1,5]diazocin- 5-yl) carbamoyl) benzo[b]thio- phen-5-yl) difluoromethyl) phosphonic acid | 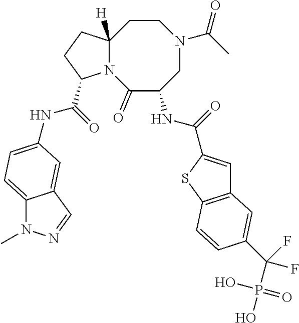 [SMILES: CC(=O)N1CC[C@H]2CC[C@@H](C(=O)Nc3ccc4c(cnn4C)c3)N2C(=O)[C@@H](NC(=O)c2cc3cc(C(F)(F)P(=O)(O)O)ccc3s2)C1] | 689 [M + H]⁺ | ¹H NMR (400 MHz, DMSO-d₆) δ 10.34- 10.22 (m, 1H), 9.08- 8.66 (m, 1H), 8.36- 8.26 (m, 2H), 8.21- 8.06 (m, 3H), 7.62- 7.51 (m, 2H), 7.37- 7.29 (m, 1H), 5.02- 4.90 (m, 1H), 4.60- 4.53 (m, 1H), 4.39- 4.25 (m, 1H), 4.14 (s, 3H), 4.07-3.37 (m, 4H), 2.38-2.24 (m, 1H), 2.20-2.10 (m, 3H), 2.10-1.72 (m, 5H). |
| 162 | ((2- (((5S,8S,10a R)-3-acetyl- 6-oxo-8-((S)- 2- phenyl- pyrrolidine-1- carbonyl) decahydropyrrolo [1,2- a][1,5]diazocin- 5- yl)carbamoyl)- 1H-indol-5-yl) difluoromethyl) phosphonic acid | 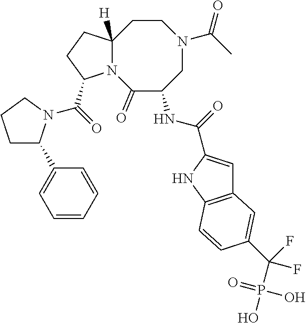 [SMILES: CC(=O)N1CC[C@H]2CC[C@@H](C(=O)N3CCC[C@H]3c3ccccc3)N2C(=O)[C@@H](NC(=O)c2cc3cc(C(F)(F)P(=O)(O)O)ccc3[nH]2)C1] | 672.4 [M + H]⁺ | ¹H NMR (400 MHz, METHANOL-d₄) δ 7.60-7.25 (m, 5H), 7.23-7.17 (m, 2H), 7.16-7.11 (m, 2H), 5.28-4.93 (m, 2H), 4.85-4.79 (m, 1H), 4.45-4.21 (m, 2H), 4.07-3.81 (m, 3H), 3.76 (s, 1H), 3.47-3.34 (m, 1H), 3.11-2.84 (m, 1H), 2.57-2.46 (m, 1H), 2.37-2.36 (m, 1H), 2.38-2.21 (m, 4H), 2.11-1.90 (m, 4H), 2.19-1.89 (m, 1H), 1.88-1.74 (m, 3H) |
| 163 | ((2- (((5S,8S,10a R)-3-acetyl- 8-((S)-2- benzylpyrrolidine- 1- carbonyl)-6- oxodeca- hydropyrrolo[1,2- a][1,5] diazocin-5- yl)carbamoyl)- 1H-indol-5-yl) difluoromethyl) phosphonic acid | 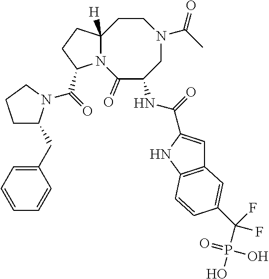 [SMILES: CC(=O)N1CC[C@H]2CC[C@@H](C(=O)N3CCC[C@H]3Cc3ccccc3)N2C(=O)[C@@H](NC(=O)c2cc3cc(C(F)(F)P(=O)(O)O)ccc3[nH]2)C1] | 686.4 [M + H]⁺ | ¹H NMR (400 MHz, METHANOL-d₄) δ 7.60-7.25 (m, 5H), 7.23-7.17 (m, 2H), 7.16-7.11 (m, 2H), 5.28-4.93 (m, 2H), 4.85-4.79 (m, 1H), 4.45-4.21 (m, 2H), 4.07-3.81 (m, 3H), 3.76 (s, 1H), 3.47-3.34 (m, 1H), 3.11-2.84 (m, 1H), 2.57-2.46 (m, 1H), 2.37-2.36 (m, 1H), 2.38-2.21 (m, 4H), 2.11-1.90 (m, 4H), 2.19-1.89 (m, 1H), 1.88-1.74 (m, |
| | | | | 4H) |
| 164 | ((2- (((5S,8S,10a R)-3-acetyl- 8-((2- methoxyphenyl) (methyl) carbamoyl)-6- oxodeca- hydropyrrolo[1, 2-a][1,5]diazoc in-5-yl) carbamoyl)- 1H-indol-5-yl) difluoromethyl) phosphonic acid | 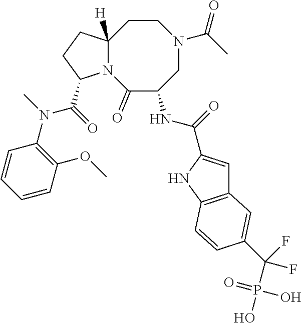 [SMILES: COc1ccccc1N(C)C(=O)[C@@H]1CC[C@@H]2CCN(C(C)=O)C[C@H](NC(=O)c3cc4cc(C(F)(F)P(=O)(O)O)ccc4[nH]3)C(=O)N21] | 662.5 [M + H]⁺ | ¹H NMR (400 MHz, METHANOL-d₄) δ 7.97-7.91 (m, 1H), 7.58-7.46 (m, 3H), 7.45-7.36 (m, 1H), 7.32-7.22 (m, 1H), 7.18-7.11 (m, 1H), 7.06-6.94 (m, 1H), 5.06-5.03 (m, 1H), 4.49-4.44 (m, 1H), 4.34-4.23 (m, 2H), 3.89 (s, 3H), 3.69-3.50 (m, 3H), 3.19-3.14 (m, 3H), 2.45-2.30 (m, 3H), 2.05-1.89 (m, 4H), 1.81-1.70 (m, 2H) |
| 165 | ((2- (((5S,8S,10a R)-3-acetyl- 8-((3- methoxyphenyl) (methyl) carbamoyl)-6- oxodeca- hydropyrrolo [1,2- a][1,5]diazocin- 5- yl)carbamoyl)- 1H-indol-5-yl) difluoromethyl) phosphonic acid | 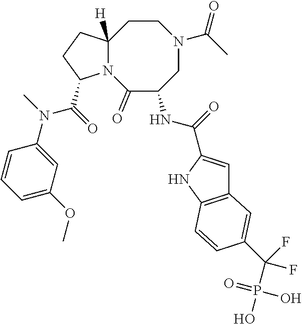 [SMILES: COc1cccc(N(C)C(=O)[C@@H]2CC[C@@H]3CCN(C(C)=O)C[C@H](NC(=O)c4cc5cc(C(F)(F)P(=O)(O)O)ccc5[nH]4)C(=O)N32)c1] | 662.3 [M + H]⁺ | ¹H NMR (400 MHz, METHANOL-d₄) δ 7.96-7.85 (m, 1H), 7.56-7.43 (m, 2H), 7.41-7.22 (m, 2H), 7.09-6.92 (m, 3H), 5.10-5.04 (m, 1H), 4.49-4.40 (m, 1H), 4.38-4.22 (m, 1H), 4.05-3.95 (m, 1H), 3.89-3.83 (m, 1H), 3.83-3.76 (m, 3H), 3.74 (d, J = 8.0 Hz, 1H), 3.59-3.51 (m, 1H), 3.27-3.20 (m, 3H), 2.36-2.25 (m, 3H), 2.07-1.92 (m, 5H), 1.84-1.73 (m, 1H) |
| 166 | ((2- (((5S,8S,10a R)-3-acetyl- 8-((4- methoxyphenyl) (methyl) carbamoyl)-6- oxodeca- hydropyrrolo [1,2-a][1,5] diazocin- 5-yl) carbamoyl)- 1H-indol-5- yl) difluoromethyl) phosphonic | 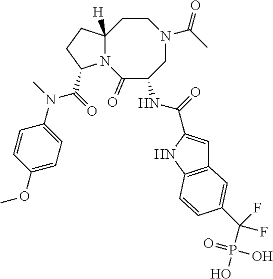 [SMILES: COc1ccc(N(C)C(=O)[C@@H]2CC[C@@H]3CCN(C(C)=O)C[C@H](NC(=O)c4cc5cc(C(F)(F)P(=O)(O)O)ccc5[nH]4)C(=O)N32)cc1] | 662.3 [M + H]⁺ | ¹H NMR (400 MHz, METHANOL-d₄) δ 8.05-7.77 (m, 1H), 7.59-7.38 (m, 2H), 7.35-7.13 (m, 3H), 6.94 (d, J = 8.8 Hz, 1H), 6.89-6.79 (m, 1H), 5.13-4.94 (m, 1H), 4.53-4.19 (m, 2H), 4.05-3.79 (m, 2H), 3.79-3.68 (m, 3H), 3.66-3.43 (m, 2H), 3.26-3.14 (m, 3H), 2.35-2.16 (m, 3H), 2.07-1.69 (m, 6H) |
| | acid | | | |
| 167 | ((2- (((5S,8S,10a R)-3-acetyl- 8-((3- cyanophenyl) (methyl) carbamoyl)-6- oxodeca- hydropyrrolo [1,2-a][1,5] diazocin-5- yl)carbamoyl)- 1H-indol-5-yl) difluoromethyl) phosphonic acid | 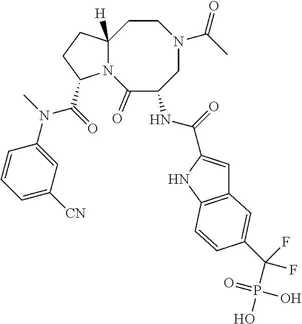 [SMILES: CC(=O)N1CC[C@H]2CC[C@@H](C(=O)N(C)c3cccc(C#N)c3)N2C(=O)[C@@H](NC(=O)c2cc3cc(C(F)(F)P(=O)(O)O)ccc3[nH]2)C1] | 657.4 [M + H]⁺ | ¹H NMR (400 MHz, METHANOL-d₄) δ 7.97-7.92 (m, 1H), 7.91-7.71 (m, 3H), 7.69-7.60 (m, 1H), 7.53-7.48 (m, 2H), 7.33-7.21 (m, 1H), 5.16-5.05 (m, 1H), 4.23 (m, 2H), 4.02- 3.91 (m, 1H), 3.90- 3.64 (m, 2H), 3.63- 3.53 (m, 1H), 3.29- 3.23 (m, 3H), 2.32- 2.25 (m, 3H), 2.09- 1.75 (m, 6H). |
| 168 | ((2- (((5S,8S,10a R)-3-acetyl- 8-((4- cyanophenyl) (methyl) carbamoyl)-6- oxodeca- hydropyrrolo [1,2-a][1,5] diazocin-5- yl)carbamoyl)- 1H-indol-5-yl) difluoromethyl) phosphonic acid | 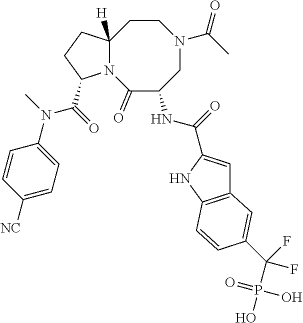 [SMILES: CC(=O)N1CC[C@H]2CC[C@@H](C(=O)N(C)c3ccc(C#N)cc3)N2C(=O)[C@@H](NC(=O)c2cc3cc(C(F)(F)P(=O)(O)O)ccc3[nH]2)C1] | 657.2 [M + H]⁺ | ¹H NMR (400 MHz, METHANOL-d₄) δ 7.98-7.90 (m, 1H), 7.87-7.75 (m, 2H), 7.72-7.60 (m, 2H), 7.54-7.45 (m, 2H), 7.33-7.24 (m, 1H), 5.16-4.97 (m, 1H), 4.46-4.25 (m, 2H), 4.06-3.82 (m, 2H), 3.81-3.71 (m, 1H), 3.64-3.54 (m, 1H), 3.32 (s, 3H), 2.31-2.25 (m, 3H), 2.09-1.89 (m, 5H), 1.84-1.75 (m, 1H) |
| 169 | ((2- (((5S,8S,10a R)-3-acetyl- 8- (ethyl(phenyl) carbamoyl)- 6-oxodeca- hydropyrrolo [1,2- a][1,5] diazocin-5- yl)carbamoyl)- 1H-indol-5-yl) difluoromethyl) phosphonic acid | 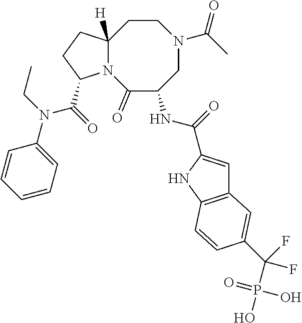 [SMILES: CCN(C(=O)[C@@H]1CC[C@@H]2CCN(C(C)=O)C[C@H](NC(=O)c3cc4cc(C(F)(F)P(=O)(O)O)ccc4[nH]3)C(=O)N21)c1ccccc1] | 646.4 [M + H]⁺ | ¹H NMR (400 MHz, METHANOL-d₄) δ 7.92-7.81 (m, 1H), 7.50-7.41 (m, 7H), 7.25 (s, 1H), 5.08-5.05 (m, 1H), 4.33-4.28 (m, 2H), 3.96-3.83 (m, 1H), 3.79-3.66 (m, 4H), 3.64-3.53 (m, 1H), 2.32-2.55 (m, 3H), 2.10- 1.91 (m, 4H), 1.75-1.67 (m, 2H), 1.16-1.11 (m, 3H) |
| 170 | ((2- (((5S,8S,10a R)-3-acetyl- 8-((1- acetylazetidin- 3- yl)(methyl) carbamoyl)-6- oxodeca- hydropyrrolo[1,2- a][1,5] diazocin-5- yl)carbamoyl)- 1H-indol-5-yl) difluoromethyl) phosphonic acid | 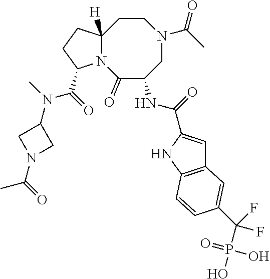 [SMILES: CC(=O)N1CC(N(C)C(=O)[C@@H]2CC[C@@H]3CCN(C(C)=O)C[C@H](NC(=O)c4cc5cc(C(F)(F)P(=O)(O)O)ccc5[nH]4)C(=O)N32)C1] | 653.2 [M + H]⁺ | ¹H NMR (400 MHz, METHANOL-d₄) δ 7.96 (s, 1H), 7.60-7.54 (m, 1H), 7.50-7.41 (m, 1H), 7.28-7.16 (m, 1H), 5.21-5.11 (m, 1H), 5.09-4.97 (m, 1H), 4.48-4.29 (m, 3H), 4.21-4.04 (m, 2H), 3.93 (dd, J = 2.4, 14 Hz, 1H), 3.85-3.55 (m, 3H), 3.53-3.39 (m, 1H), 3.22-3.15 (m, 2H), 3.08-3.00 (m, 1H), 2.21 (s, 6H), 1.99- 1.85 (m, 6H) |
| 171 | ((2- (((5S,8S,10a R)-3-acetyl- 8-((1- acetylazetidin- 3- yl)(methyl) carbamoyl)-6- oxodecahydro- pyrrolo[1,2- a][1,5] diazocin-5- yl)carbamoyl) benzo[b]thio- phen-5- yl) difluoromethyl) | 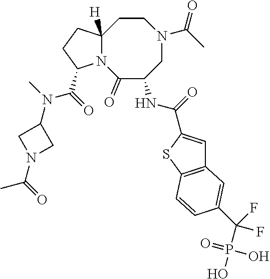 [SMILES: CC(=O)N1CC(N(C)C(=O)[C@@H]2CC[C@@H]3CCN(C(C)=O)C[C@H](NC(=O)c4cc5cc(C(F)(F)P(=O)(O)O)ccc5s4)C(=O)N32)C1] | 670 [M + H]⁺ | ¹H NMR (400 MHz, METHANOL-d₄) δ 8.13-8.03 (m, 2H), 7.99-7.92 (m, 1H), 7.66-7.58 (m, 1H), 5.20-4.91 (m, 2H), 4.90 (s, 1H), 4.43-4.21 (m, 3H), 4.20-3.98 (m, 2H), 3.96-3.84 (m, 1H), 3.82-3.68 (m, 1H), 3.66-3.42 (m, 2H), 3.16-3.10 (m, 2H), 3.03-2.93 (m, 1H), 2.40-2.26 (m, 1H), 2.24-2.11 (m, 4H), 1.93 (s, 1H), 1.91- 1.71 (m, 6H) |
| | phosphonic | | | |
| | acid | | | |
| 172 | ((2- (((5S,8S,10a R)-3-acetyl- 6-oxo-8- (quinazolin- 6-ylcarbamoyl) decahydropyrrolo [1,2-a][1,5] diazocin- 5-yl) carbamoyl) benzo[b]thio- phen-5-yl) difluoromethyl) phosphonic acid | 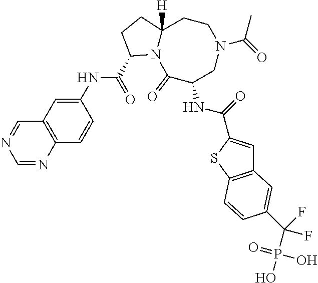 [SMILES: CC(=O)N1CC[C@H]2CC[C@@H](C(=O)Nc3ccc4ncncc4c3)N2C(=O)[C@@H](NC(=O)c2cc3cc(C(F)(F)P(=O)(O)O)ccc3s2)C1] | 687.2 [M + H]⁺ | ¹H NMR (400 MHz, METHANOL-d₄) δ 8.52-8.35 (m, 1H), 8.18-8.11 (m, 1H), 8.08-7.89 (m, 2H), 7.76-7.53 (m, 2H), 7.25-6.92 (m, 1H), 6.17-5.90 (m, 1H), 5.35-5.16 (m, 1H), 4.63-4.39 (m, 2H), 3.88-3.45 (m, 3H), 2.52-2.35 (m, 1H), 2.34-2.16 (m, 6H), 2.15-2.01 (m, 2H), 2.00-1.80 (m, 2H) |
| 173 | ((2- (((5S,8S,10a R)-3-acetyl- 8- (naphthalen- 2- ylcarbamoyl)- 6-oxodeca- hydropyrrolo [1,2-a][1,5] diazocin-5- yl)carbamoyl) benzo[b]thio- phen-5-yl) difluoromethyl) phosphonic acid | 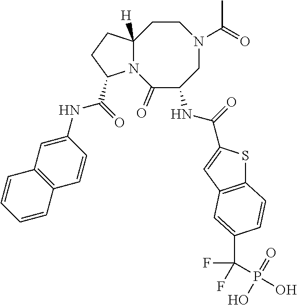 [SMILES: CC(=O)N1CC[C@H]2CC[C@@H](C(=O)Nc3ccc4ccccc4c3)N2C(=O)[C@@H](NC(=O)c2cc3cc(C(F)(F)P(=O)(O)O)ccc3s2)C1] | 685.0 [M + H]⁺ | ¹H NMR (400 MHz, METHANOL-d₄) δ 8.36-8.20 (m, 1H), 8.14-8.04 (m, 1H), 8.03-7.81 (m, 2H), 7.80-7.70 (m, 3H), 7.69-7.59 (m, 1H), 7.57-7.49 (m, 1H), 7.47-7.33 (m, 2H), 5.29-4.95 (m, 1H), 4.79-4.58 (m, 1H), 4.54-4.29 (m, 1H), 4.08-3.54 (m, 4H), 2.46-2.31 (m, 1H), 2.30-2.05 (m, 5H), 2.05-1.72 (m, 3H) |
| 174 | ((2- (((5S,8S,10a R)-3-acetyl- 8-(cinnolin- 6- ylcarbamoyl)- 6-oxodeca- hydropyrrolo [1,2-a][1,5] diazocin-5-yl) carbamoyl) benzo[b]thio- phen-5-yl) difluoromethyl) phosphonic acid | 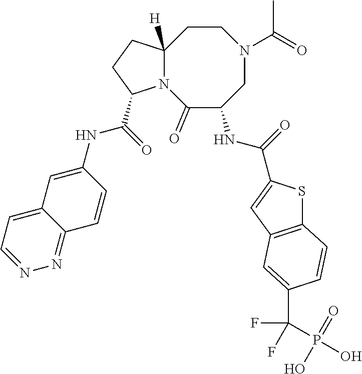 [SMILES: CC(=O)N1CC[C@H]2CC[C@@H](C(=O)Nc3ccc4nnccc4c3)N2C(=O)[C@@H](NC(=O)c2cc3cc(C(F)(F)P(=O)(O)O)ccc3s2)C1] | 687.0 [M + H]⁺ | ¹H NMR (400 MHz, METHANOL-d₄) δ 9.30-9.09 (m, 1H), 8.77 (d, J = 2.0 Hz, 1H), 8.66-8.50 (m, 1H), 8.48-8.33 (m, 1H), 8.14-8.03 (m, 2H), 8.03-7.87(m, 2H), 7.75-7.56 (m, 1H), 5.42-5.15 (m, 1H), 4.71-4.62 (m, 1H), 4.61-4.47 (m, 1H), 4.08-3.51 (m, 4H), 2.53-2.36 (m, 1H), 2.35-2.19 (m, 5H), 2.19-2.06 (m, 1H), 1.97-1.72 (m, 2H) |
| 175 | ((2- (((5S,8S,10a R)-8-((1H- indazol-5-yl) carbamoyl)- 3-acetyl-6- oxodecahydro- pyrrolo[1,2- a][1,5] diazocin-5- yl)carbamoyl) benzo[b]thio- phen-5-yl) difluoromethyl) phosphonic acid | 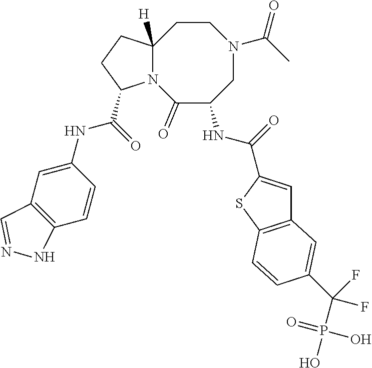 [SMILES: CC(=O)N1CC[C@H]2CC[C@@H](C(=O)Nc3ccc4[nH]ncc4c3)N2C(=O)[C@@H](NC(=O)c2cc3cc(C(F)(F)P(=O)(O)O)ccc3s2)C1] | 675 [M + H]⁺ | ¹H NMR (400 MHz, METHANOL-d₄) δ 8.09-8.12 (d, J = 10.8 Hz, 2H), 8.07-7.97 (m, 2H), 7.95 (d, J = 8.4 Hz, 1H), 7.69-7.61 (m, 1H), 7.51-7.39 (m, 2H), 5.33-5.00 (m, 1H), 4.74-4.56 (m, 1H), 4.41 (dd, J = 4.4, 7.6 Hz, 1H), 3.97-3.56 (m, 4H), 2.42-2.27 (m, 1H), 2.26-2.04 (m, 6H), 2.02-1.81 (m, 2H) |
| 176 | ((2- (((5S,8S,10a R)-3-acetyl- 8-((3- (cyanomethy 1)-[1,1′- biphenyl]-4- yl)carbamoyl)- 6-oxodeca- hydropyrrolo [1,2- a][1,5] diazocin-5-yl) carbamoyl) benzo[b]thio- phen-5-yl) difluoromethyl) | 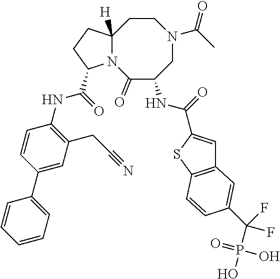 [SMILES: CC(=O)N1CC[C@H]2CC[C@@H](C(=O)Nc3ccc(-c4ccccc4)cc3CC#N)N2C(=O)[C@@H](NC(=O)c2cc3cc(C(F)(F)P(=O)(O)O)ccc3s2)C1] | 750.3 [M + H]⁺ | ¹H NMR (400 MHz, METHANOL-d₄) δ 8.19-8.15 (m, 2H), 8.04-7.99 (m, 1H), 7.73 (s, 1H), 7.71-7.66 (m, 1H), 7.65-7.56 (m, 3H), 7.45 (m, 2H), 7.40 (d, J = 8.4 Hz, 1H), 7.38-7.35 (m, 1H), 5.20 (dd, J = 3.6, 10.8 Hz, 1H), 4.66 (m, 1H), 4.56-4.40 (m, 1H), 3.99 (s, 1H), 3.97-3.83 (m, 2H), 3.75-3.64 (m, 2H), 2.50-2.38 (m, 1H), 2.33-2.12 (m, 6H), 2.09-1.82 (m, |
| | phosphonic | | | 3H) |
| | acid | | | |
| 177 | ((2- (((5S,8S,10a R)-3-acetyl- 8-((3-(2- amino-2- oxoethyl)- [1,1′- biphenyl]-4- yl)carbamoyl)- 6-oxodeca- hydropyrrolo [1,2- a][1,5]diazocin- 5- yl)carbamoyl) benzo[b]thio- phen-5-yl) difluoromethyl) phosphonic acid | 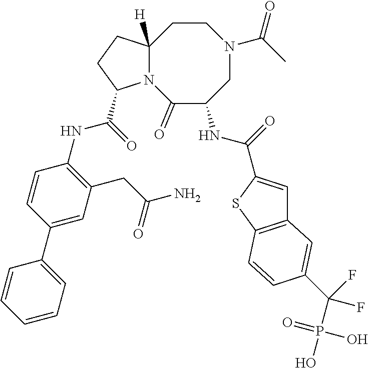 [SMILES: CC(=O)N1CC[C@H]2CC[C@@H](C(=O)Nc3ccc(-c4ccccc4)cc3CC(N)=O)N2C(=O)[C@@H](NC(=O)c2cc3cc(C(F)(F)P(=O)(O)O)ccc3s2)C1] | 768.2 [M + H]⁺ | ¹H NMR (400 MHz, METHANOL-d₄) δ 8.18-8.10 (m, 2H), 8.03-7.95 (m, 1H), 7.71-7.64 (m, 1H), 7.64-7.46 (m, 5H), 7.45-7.38 (m, 2H), 7.35-7.28 (m, 1H), 5.21-5.10 (m, 1H), 4.71-4.64 (m, 1H), 4.51-4.35 (m, 1H), 4.05-3.85 (m, 1H), 3.81-3.70 (m, 2H), 3.69-3.53 (m, 3H), 2.45-2.45 (m, 1H), 2.49-2.37 (m, 1H), 2.32-2.24 (m, 1H), 2.22 (s, 3H), 2.15 (s, 1H), 2.10-1.94 (m, 2H), 1.94-1.81 (m, 1H) |
| 178 | ((2- (((5S,8S,10a R)-3-acetyl- 8-((3-nitro- [1,1′- biphenyl]-4- yl)carbamoyl)- 6-oxodeca- hydropyrrolo a][1,5] diazocin-5-yl) carbamoyl) benzo[b]thio- phen-5-yl) difluoromethyl) phosphonic acid | 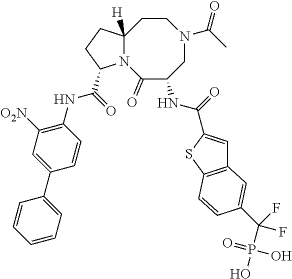 [SMILES: CC(=O)N1CC[C@H]2CC[C@@H](C(=O)Nc3ccc(-c4ccccc4)cc3[N+](=O)[O-])N2C(=O)[C@@H](NC(=O)c2cc3cc(C(F)(F)P(=O)(O)O)ccc3s2)C1] | 756.3 [M + H]⁺ | ¹H NMR (400 MHz, METHANOL-d₄) δ 8.33 (d, J = 2.4 Hz, 1H), 8.29-8.25 (m, 1H), 8.17-8.11 (m, 2H), 8.00-7.85 (m, 2H), 7.71-7.64 (m, 3H), 7.52-7.37 (m, 3H), 5.27-5.12 (m, 1H), 4.79-4.73 (m, 1H), 4.53-4.41 (m, 1H), 3.90-3.80 (m, 1H), 3.78-3.59 (m, 3H), 2.44-2.32 (m, 1H), 2.31-2.20 (m, 2H), 2.20-2.15 (m, 3H), 2.13-2.00 (m, |
| | | | | 1H), 1.98-1.82 (m, |
| | | | | 2H) |
| 179 | ((2- (((5S,8S,10a R)-3-acetyl- 8-((2-nitro- [1,1′- biphenyl]-4- yl)carbamoyl)- 6-oxodeca- hydropyrrolo [1,2- a][1,5]diazocin- 5- yl)carbamoyl) benzo[b]thio- phen-5-yl) difluoromethyl) phosphonic | 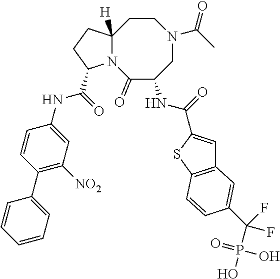 [SMILES: CC(=O)N1CC[C@H]2CC[C@@H](C(=O)Nc3ccc(-c4ccccc4)c([N+](=O)[O-])c3)N2C(=O)[C@@H](NC(=O)c2cc3cc(C(F)(F)P(=O)(O)O)ccc3s2)C1] | 756.1 [M + H]⁺ | ¹H NMR (400 MHz, METHANOL-d₄) δ 8.37 (d, J = 2.0 Hz, 1H), 8.16 (s, 1H), 8.13 (s, 1H), 8.03-7.96 (m, 1H), 7.76 (dd, J = 2.4, 8.4 Hz, 1H), 7.73-7.67 (m, 1H), 7.45-7.38 (m, 4H), 7.33-7.28 (m, 2H), 5.25 (dd, J = 4.4, 11.2 Hz, 1H), 4.67- 4.57 (m, 1H), 4.56- 4.42 (m, 1H), 4.00- 3.84 (m, 2H), 3.83- 3.69 (m, 2H), 2.45- 2.36 (m, 1H), 2.29- 2.25 (m, 3H), 2.25- |
| | acid | | | 2.07 (m, 3H), 2.00- |
| | | | | 1.89 (m, 2H) |
| 180 | ((2- (((5S,8S,10a R)-3-acetyl- 6-oxo-8-((2- oxo-6- phenyl-1,2- dihydropyridin- 3-yl) carbamoyl) decahydropyrrolo [1,2- a][1,5] diazocin-5- yl)carbamoyl)- 1H-indol-5-yl) difluoromethyl) phosphonic | 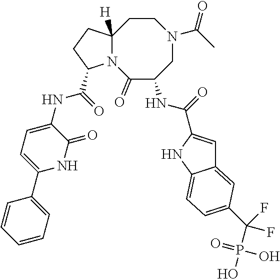 [SMILES: CC(=O)N1CC[C@H]2CC[C@@H](C(=O)Nc3ccc(-c4ccccc4)[nH]c3=O)N2C(=O)[C@@H](NC(=O)c2cc3cc(C(F)(F)P(=O)(O)O)ccc3[nH]2)C1] | 711.4 [M + H]⁺ | ¹H NMR (400 MHz, METHANOL-d₄) δ 8.50-8.38 (m, 1H), 7.91-7.80 (m, 1H), 7.63 (d, J = 7.2 Hz, 2H), 7.52-7.33 (m, 5H), 7.18 (s, 1H), 6.58- 6.49 (m, 1H), 5.31- 5.21 (m, 1H), 4.84- 4.77 (m, 1H), 4.39 (d, J = 4.0 Hz, 1H), 3.90- 3.56 (m, 4H), 2.38- 2.23 (m, 1H), 2.22- 2.09 (m, 5H), 2.07- 1.97 (m, 1H), 1.96- 1.78 (m, 2H) |
| | acid | | | |
| 181 | ((2- (((5S,8S,10a R)-3-acetyl- 6-oxo-8- (phenyl- carbamoyl) decahydropyrrolo [1,2-a][1,5] diazocin- 5-yl) carbamoyl) benzo[b]thio- phen-5-yl) difluoromethyl) phosphonic acid | 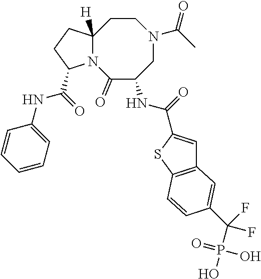 [SMILES: CC(=O)N1CC[C@H]2CC[C@@H](C(=O)Nc3ccccc3)N2C(=O)[C@@H](NC(=O)c2cc3cc(C(F)(F)P(=O)(O)O)ccc3s2)C1] | 635.0 [M + H]⁺ | ¹H NMR (400 MHz, METHANOL-d₄) δ 8.17-8.08 (m, 2H), 8.05-7.98 (m, 1H), 7.68 (d, J = 8.4 Hz, 1H), 7.62-7.53 (m, 2H), 7.34-7.23 (m, 2H), 7.13-7.01 (m, 1H), 5.23-5.12 (m, 1H), 4.65-4.55 (m, 1H), 4.49-4.37 (m, 1H), 4.06-3.80 (m, 2H), 3.79-3.64 (m, 2H), 2.45-2.34 (m, 1H), 2.26-2.22 (m, 3H), 2.20-2.01 (m, 3H), 2.00-1.79 (m, |
| | | | | 2H) |
| 182 | ((2- (((5S,8S,10a R)-3-acetyl- 8-(((3S,4S)- 1-acetyl-4- hydroxy- pyrrolidin-3- yl)carbamoyl)- 6- oxodecahydro- pyrrolo[1,2- a][1,5] diazocin-5- yl)carbamoyl) benzo[b]thio- phen-5-yl) difluoromethyl) | 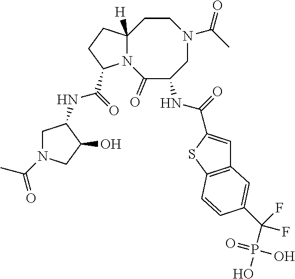 [SMILES: CC(=O)N1CC[C@H]2CC[C@@H](C(=O)N[C@H]3CN(C(C)=O)C[C@@H]3O)N2C(=O)[C@@H](NC(=O)c2cc3cc(C(F)(F)P(=O)(O)O)ccc3s2)C1] | 686.4 [M + H]⁺ | ¹H NMR (400 MHz, METHANOL-d₄) δ 8.21-8.09 (m, 2H), 8.05-7.96 (m, 1H), 7.70 (d, J = 8.0 Hz, 1H), 5.17-5.02 (m, 1H), 4.49-4.34 (m, 2H), 4.25-4.11 (m, 2H), 4.02-3.86 (m, 2H), 3.84-3.70 (m, 2H), 3.68-3.56 (m, 2H), 3.54-3.41 (m, 2H), 2.33-2.21 (m, 4H), 2.21-2.14 (m, 2H), 2.12-1.95 (m, 5H), 1.95-1.79 (m, 2H) |
| | phosphonic | | | |
| | acid | | | |
| 183 | ((2- (((5S,8S,10a R)-3-acetyl- 8-(((3S,4S)- 1-acetyl-4- hydroxypyrolidin- 3- yl)(methyl) carbamoyl)-6- oxodeca- ohydrpyrrolo[1,2- a][1,5]diazocin- 5- yl)carbamoyl) benzo[b]thio- phen-5-yl) difluoromethyl) | 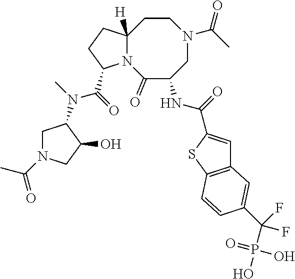 [SMILES: CC(=O)N1CC[C@H]2CC[C@@H](C(=O)N(C)[C@H]3CN(C(C)=O)C[C@@H]3O)N2C(=O)[C@@H](NC(=O)c2cc3cc(C(F)(F)P(=O)(O)O)ccc3s2)C1] | 700.1 [M + H]⁺ | ¹H NMR (400 MHz, METHANOL-d₄) δ 8.19-8.08 (m, 2H), 8.06-7.96 (m, 1H), 7.72-7.63 (m, 1H), 5.16-5.02 (m, 1H), 4.80-4.67 (m, 1H), 4.66-4.52 (m, 1H), 4.51-4.33 (m, 2H), 4.08-3.93 (m, 1H), 3.91-3.47 (m, 6H), 3.45-3.33 (m, 1H), 3.26-3.07 (m, 3H), 2.93-2.80 (m, 1H), 2.52-2.38 (m, 1H), 2.32-2.17 (m, 4H), 2.08-1.83 (m, 7H). |
| | phosphonic | | | |
| | acid | | | |
| 184 | ((2- (((5S,8S,10a R)-3-acetyl- 8-(((3S,4S)- 1-acetyl-4- methoxy- pyrrolidin-3- yl)carbamoyl)- 6-oxodeca- hydropyrrolo [1,2-a][1,5] diazocin-5- yl)carbamoyl) benzo[b]thio- phen-5-yl) difluoromethyl) phosphonic acid | 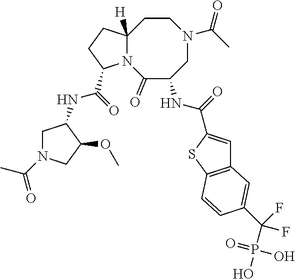 [SMILES: CO[C@H]1CN(C(C)=O)C[C@@H]1NC(=O)[C@@H]1CC[C@@H]2CCN(C(C)=O)C[C@H](NC(=O)c3cc4cc(C(F)(F)P(=O)(O)O)ccc4s3)C(=O)N21] | 700.0 [M + H]⁺ | ¹H NMR (400 MHz, METHANOL-d₄) δ 8.21-8.09 (m, 2H), 8.08-7.98 (m, 1H), 7.75-7.63 (m, 1H), 5.16-5.04 (m, 1H), 4.48-4.27 (m, 3H), 4.02-3.89 (m, 1H), 3.88-3.67 (m, 4H), 3.66-3.46 (m, 5H), 3.45-3.39 (m, 3H), 2.32-2.26 (m, 3H), 2.24 (m, 1H), 2.21- 2.11 (m, 1H), 2.09- 2.05 (m, 3H), 2.03- 1.96 (m, 2H), 1.91- 1.80 (m, 1H) |
| 185 | ((2- (((5S,8S,10a R)-3-acetyl- 8-((4-(tert- butyl)phenyl) (methyl) carbamoyl)-6- oxodecahydro- pyrrolo[1,2- a][1,5] diazocin-5- yl)carbamoyl) benzo[b]thio- phen-5- yl)difluoromethyl) phosphonic acid | 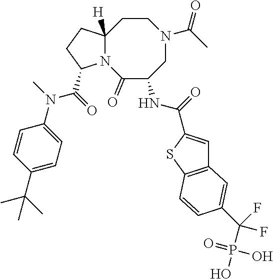 [SMILES: CC(=O)N1CC[C@H]2CC[C@@H](C(=O)N(C)c3ccc(C(C)(C)C)cc3)N2C(=O)[C@@H](NC(=O)c2cc3cc(C(F)(F)P(=O)(O)O)ccc3s2)C1] | 705.4 [M + H]⁺ | ¹H NMR (400 MHz, METHANOL-d₄) δ 8.21-8.08 (m, 2H), 8.05-7.94 (m, 1H), 7.74-7.63 (m, 1H), 7.52-7.41 (m, 2H), 7.40-7.30 (m, 2H), 5.07-4.99 (m, 1H), 4.47-4.25 (m, 2H), 4.02 (dd, J = 3.6, 14.4 Hz, 1H), 3.89-3.81 (m, 1H), 3.68-3.47 (m, 2H), 3.27-3.20 (m, 3H), 2.34-2.25 (m, 3H), 2.09-1.87 (m, 5H), 1.84-1.70 (m, 1H), 1.33-1.27 (m, 9H) |
| 186 | ((2- (((5S,8S,10a R)-3-acetyl- 8-((S)-2- benzyl- pyrrolidine-1- carbonyl)-6- oxodeca- hydropyrrolo[1,2- a][1,5]diazoc in-5- yl)carbamoyl) benzo[b]thio- phen-5-yl) difluoromethyl) phosphonic acid | 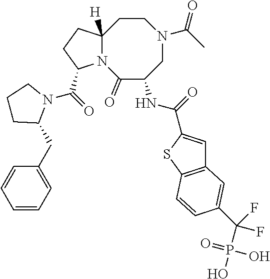 [SMILES: CC(=O)N1CC[C@H]2CC[C@@H](C(=O)N3CCC[C@H]3Cc3ccccc3)N2C(=O)[C@@H](NC(=O)c2cc3cc(C(F)(F)P(=O)(O)O)ccc3s2)C1] | 703.3 [M + H]⁺ | ¹H NMR (400 MHz, METHANOL-d₄) δ 8.20-7.96 (m, 3H), 7.73-7.61 (m, 1H), 7.33-7.20 (m, 5H), 5.09 (dd, J = 3.2, 10.8 Hz, 1H), 4.79-4.72 (m, 1H), 4.43-4.27 (m, 2H), 4.16-4.04 (m, 1H), 4.02-3.91 (m, 1H), 3.87-3.75 (m, 1H), 3.63-3.44 (m, 3H), 3.12-3.02 (m, 1H), 2.74-2.61 (m, 1H), 2.47-2.34 (m, 3H), 2.27 (s, 1H), 2.23- 2.11 (m, 2H), 2.09- 2.01 (m, 1H), 1.98- 1.86 (m, 4H), 1.85- 1.76 (m, 2H) |
| 187 | ((2- (((5S,8S,10a R)-3-acetyl- 8-((4- chlorophenyl) (methyl) carbamoyl)-6- oxodecahydro- pyrrolo[1,2- a][1,5] diazocin-5- yl)carbamoyl) benzo[b]thio- phen-5-yl) difluoromethyl) phosphonic acid | 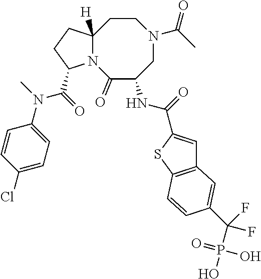 [SMILES: CC(=O)N1CC[C@H]2CC[C@@H](C(=O)N(C)c3ccc(Cl)cc3)N2C(=O)[C@@H](NC(=O)c2cc3cc(C(F)(F)P(=O)(O)O)ccc3s2)C1] | 683.3 [M + H]⁺ | ¹H NMR (400 MHz, METHANOL-d₄) δ 8.22-8.13 (m, 2H), 8.03 (d, J = 8.4 Hz, 1H), 7.71 (d, J = 8.0 Hz, 1H), 7.52-7.40 (m, 4H), 5.06 (s, 1H), 4.42-4.33 (m, 1H), 4.31-4.25 (m, 1H), 4.05-3.95 (m, 1H), 3.89-3.73 (m, 2H), 3.48 (d, J = 1.6 Hz, 2H), 3.28-3.20 (m, 3H), 2.35-2.22 (m, 3H), 2.04-1.92 (m,4H), 1.37-1.26 (m, 2H) |
| 188 | ((2- (((5S,8S,10a R)-3-acetyl- 8-((3,4- dichlorophenyl) (methyl) carbamoyl)-6- oxodeca- hydropyrrolo[1,2- a][1,5] diazocin-5- yl)carbamoyl) benzo[b]thio- phen-5- yl)difluoromethyl) phosphonic acid | 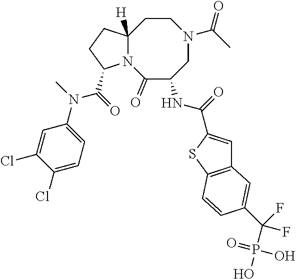 [SMILES: CC(=O)N1CC[C@H]2CC[C@@H](C(=O)N(C)c3ccc(Cl)c(Cl)c3)N2C(=O)[C@@H](NC(=O)c2cc3cc(C(F)(F)P(=O)(O)O)ccc3s2)C1] | 717.4 [M + H]⁺ | ¹H NMR (400 MHz, METHANOL-d₄) δ 8.23-8.10 (m, 2H), 8.06-7.93 (m, 1H), 7.78-7.54 (m, 3H), 7.51-7.30 (m, 1H), 5.17-5.02 (m, 1H), 4.52-4.20 (m, 2H), 4.10-3.94 (m, 1H), 3.90-3.54 (m, 3H), 3.28-3.19 (m, 3H), 2.37-2.20 (m, 3H), 2.10-1.91 (m, 4H), 1.86-1.69 (m, 1H), 1.37-1.22 (m, 1H) |
| 189 | ((2- (((5S,8S,10a R)-3-acetyl- 8-(cinnolin- 7- ylcarbamoyl)- 6-oxodeca- hydropyrrolo [1,2- a][1,5] diazocin-5- yl)carbamoyl) benzo[b]thio- phen-5-yl) difluoromethyl) phosphonic acid | 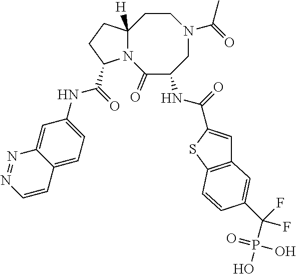 [SMILES: CC(=O)N1CC[C@H]2CC[C@@H](C(=O)Nc3ccc4ccnnc4c3)N2C(=O)[C@@H](NC(=O)c2cc3cc(C(F)(F)P(=O)(O)O)ccc3s2)C1] | 687.2 [M + H]⁺ | ¹H NMR (400 MHz, DMSO-d₆) δ 9.29- 9.27 (m, 1H), 8.87 (s, 1H), 8.76 (d, J = 7.2 Hz, 1H), 8.30 (s, 1H), 8.16-8.12 (m, 2H), 8.08-8.02 (m, 2H), 7.92 (dd, J = 1.6, 8.8 Hz, 1H), 7.59 (d, J = 8.4 Hz, 1H), 5.22-4.78 (m, 2H), 4.66-4.48 (m, 2H), 4.44-4.31 (m, 2H), 3.99-3.84 (m, 4H), 2.33 (s, 2H), 2.16 (s, 4H), 1.95-1.87 (m, 2H) |
| 190 | ((2- (((5S,8S,10a R)-3-acetyl- 8-(methyl(4- morpholino- phenyl) carbamoyl)-6- oxodeca- hydropyrrolo[1,2- a][1,5]diazocin- 5- yl)carbamoyl) benzo[b]thio- phen-5-yl) difluoromethyl) phosphonic acid | 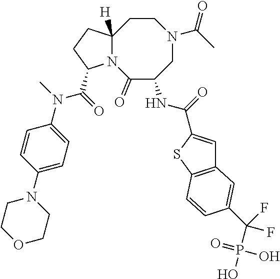 [SMILES: CC(=O)N1CC[C@H]2CC[C@@H](C(=O)N(C)c3ccc(N4CCOCC4)cc3)N2C(=O)[C@@H](NC(=O)c2cc3cc(C(F)(F)P(=O)(O)O)ccc3s2)C1] | 734.4 [M + H]⁺ | ¹H NMR (400 MHz, METHANOL-d₄) δ 8.15-8.06 (m, 1H), 7.96-7.85 (m, 1H), 7.82-7.56 (m, 2H), 7.45-7.01 (m, 4H), 5.14-4.88 (m, 1H), 4.56-4.25 (m, 3H), 4.01 (dd, J = 3.2, 14.4 Hz, 1H), 3.91 (d, J = 17.2 Hz, 5H), 3.61- 3.49 (m, 1H), 3.29- 3.10 (m, 6H), 2.35- 2.25 (m, 3H), 2.16- 1.72 (m, 6H), 1.34- 1.28 (m, 1H) |
| 191 | ((2- (((5S,8S,10a R)-3-acetyl- 8-(cinnolin- 6- yl(methyl) carbamoyl)-6- oxodeca- hydropyrrolo[1,2- a][1,5]diazocin- 5-yl) carbamoyl) benzo[b]thio- phen-5-yl) difluoromethyl) phosphonic acid | 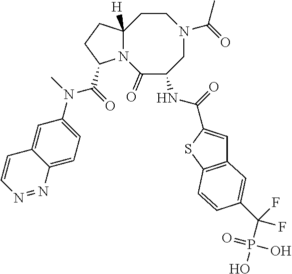 [SMILES: CC(=O)N1CC[C@H]2CC[C@@H](C(=O)N(C)c3ccc4nnccc4c3)N2C(=O)[C@@H](NC(=O)c2cc3cc(C(F)(F)P(=O)(O)O)ccc3s2)C1] | 701 [M + H]⁺ | ¹H NMR (400 MHz, METHANOL-d₄) δ 9.38-9.23 (m, 1H), 8.60-8.46 (m, 1H), 8.39-8.28 (m, 1H), 8.20-8.09 (m, 3H), 8.05-7.94 (m, 1H), 7.74-7.65 (m, 1H), 7.51-7.32 (m, 1H), 5.15-5.00 (m, 1H), 4.68-4.56 (m, 1H), 4.47-4.29 (m, 1H), 3.97 (dd, J = 2.8, 13.2 Hz, 1H), 3.84 (d, J = 6.4 Hz, 1H), 3.78-3.62 (m, 2H), 3.52-3.39 (m, 3H), 2.33-2.21 (m, |
| | | | | 3H), 2.19-1.69 (m, |
| | | | | 6H) |
| 192 | ((2- (((5S,8S,10a R)-3-acetyl- 6-oxo-8-(4- (piperidine- 1- carbonyl) piperidine-1- carbonyl) decahydropyrrolo [1,2- a][1,5] diazocin-5- yl)carbamoyl) benzo[b]thio- phen-5-yl) difluoromethyl) | 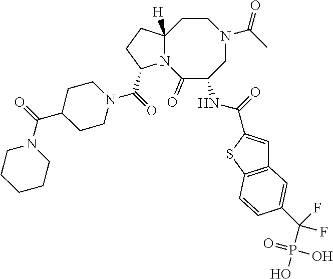 [SMILES: CC(=O)N1CC[C@H]2CC[C@@H](C(=O)N3CCC(C(=O)N4CCCCC4)CC3)N2C(=O)[C@@H](NC(=O)c2cc3cc(C(F)(F)P(=O)(O)O)ccc3s2)C1] | 738.2 [M + H]⁺ | ¹H NMR (400 MHz, METHANOL-d₄) δ 8.20-8.10 (m, 2H), 8.04 (d, J = 8.4 Hz, 1H), 7.69 (d, J = 9.2 Hz, 1H), 5.14-4.91 (m, 2H), 4.49 (d, J = 10.4 Hz,2H), 4.40- 4.24 (m, 2H), 4.13 (s, 1H), 3.90-3.84 (m, 1H), 3.66-3.50 (m, 6H), 3.01 (td, J = 7.2, 10.4 Hz, 2H), 2.89- 2.72 (m, 2H), 2.34 (d, J = 4.4 Hz, 3H),2.27- 2.01 (m, 4H), 1.99- 1.81 (m, 4H), 1.70- |
| | phosphonic | | | 1.55 (m, 6H) |
| | acid | | | |
| 193 | ((2- (((5S,8S,10a R)-3-acetyl- 6-oxo-8-((S)- 3- (piperidine- 1- carbonyl) pyrrolidine-1- carbonyl) decahydropyrrolo [1,2- a][1,5]diazocin- 5-yl) carbamoyl) benzo[b]thio- phen-5-yl) | 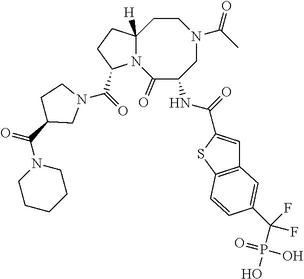 [SMILES: CC(=O)N1CC[C@H]2CC[C@@H](C(=O)N3CC[C@H](C(=O)N4CCCCC4)C3)N2C(=O)[C@@H](NC(=O)c2cc3cc(C(F)(F)P(=O)(O)O)ccc3s2)C1] | 724.1 [M + H]⁺ | ¹H NMR (400 MHz, METHANOL-d₄) δ 8.09-7.98 (m, 2H), 7.92 (d, J = 8.4 Hz, 1H), 7.58 (d, J = 8.4 Hz, 1H), 5.00-4.83 (m, 1H), 4.68-4.59 (m, 1H), 4.37-4.17 (m, 1H), 4.03-3.82 (m, 2H), 3.79-3.70 (m, 1H), 3.65-3.57 (m, 1H), 3.52-3.31 (m, 9H), 2.37-2.23 (m, 1H), 2.21 (d, J = 2.0 Hz, 2H), 2.13 (s, 3H), 2.03-1.70 (m, 5H), 1.62-1.39 (m, 6H) |
| | difluoromethyl) | | | |
| | phosphonic | | | |
| | acid | | | |
| 194 | ((2- (((5S,8S,10a R)-3-acetyl- 6-oxo-8- ((R)-3- (piperidine- 1- carbonyl) pyrrolidine-1- carbonyl) decahydropyrrolo [1,2- a][1,5]diazocin- 5- yl)carbamoyl) benzo[b]thio- phen-5-yl) difluoromethyl) phosphonic acid | 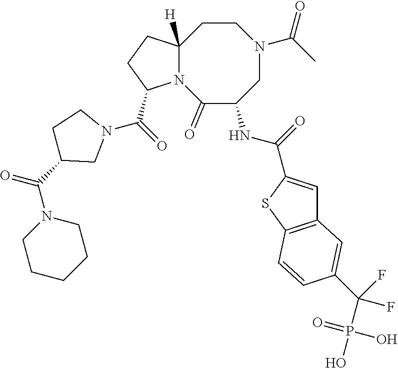 [SMILES: CC(=O)N1CC[C@H]2CC[C@@H](C(=O)N3CC[C@@H](C(=O)N4CCCCC4)C3)N2C(=O)[C@@H](NC(=O)c2cc3cc(C(F)(F)P(=O)(O)O)ccc3s2)C1] | 724.1 [M + H]⁺ | ¹H NMR (400 MHz, METHANOL-d₄) δ 8.21-8.09 (m, 2H), 8.08-8.00 (m, 1H), 7.72-7.65 (m, 1H), 5.09-4.94 (m, 1H), 4.76-4.67 (m, 1H), 4.48-4.29 (m, 1H), 4.08-3.97 (m, 1H), 3.87-3.64 (m, 4H), 3.62-3.38 (m, 8H), 2.46-2.36 (m, 1H), 2.32 (s, 2H), 2.28-2.16 (m, 3H), 2.14-1.81 (m, 5H), 1.74-1.52 (m, 6H) |
| 195 | ((2- (((5S,8S,10a R)-3-acetyl- 8-(3-(4- methylpiperazine- 1- carbonyl) azetidine-1- carbonyl)-6- oxodecahydro- pyrrolo[1,2- a][1,5]diazocin- 5-yl) carbamoyl) benzo[b]thio- phen-5-yl) difluoromethyl) | 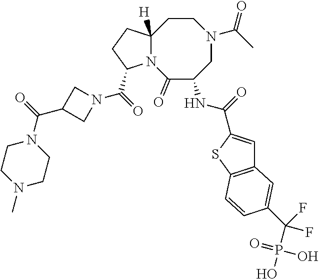 [SMILES: CC(=O)N1CC[C@H]2CC[C@@H](C(=O)N3CC(C(=O)N4CCN(C)CC4)C3)N2C(=O)[C@@H](NC(=O)c2cc3cc(C(F)(F)P(=O)(O)O)ccc3s2)C1] | 725.1 [M + H]⁺ | ¹H NMR (400 MHz, METHANOL-d₄) δ 8.19-8.11 (m, 1H), 8.10-8.00 (m, 1H), 8.00-7.84 (m, 1H), 7.75-7.62 (m, 1H), 5.17-5.06 (m, 1H), 4.73-4.58 (m, 1H), 4.56-4.42 (m, 3H), 4.41-4.30 (m, 2H), 4.23-4.13 (m, 1H), 4.09-3.97 (m, 2H), 3.95-3.74 (m, 2H), 3.73-3.60 (m, 2H), 3.60-3.44 (m, 3H), 2.98-2.88 (m, 1H), 2.88-2.81 (m, 2H), |
| | phosphonic | | | 2.80-2.67 (m, 2H), |
| | acid | | | 2.35-2.20 (m, 6H), |
| | | | | 2.03-1.81 (m, 4H) |
| 196 | ((2- (((5S,8S,10a R)-3-acetyl- 6-oxo-8-((3- (thiazol-2- ylmethyl)- [1,1′- biphenyl]-4- yl)carbamoyl) decahydropyrrolo [1,2- a][1,5]diazocin- 5- yl)carbamoyl) benzo[b]thio- phen-5-yl) difluoromethyl) | 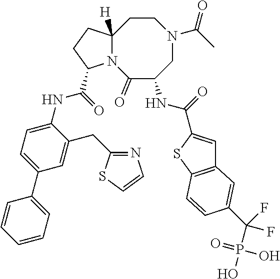 [SMILES: CC(=O)N1CC[C@H]2CC[C@@H](C(=O)Nc3ccc(-c4ccccc4)cc3Cc3nccs3)N2C(=O)[C@@H](NC(=O)c2cc3cc(C(F)(F)P(=O)(O)O)ccc3s2)C1] | 808.5 [M + H]⁺ | ¹H NMR (400 MHz, METHANOL-d₄) δ 8.19-8.09 (m, 2H), 8.02-7.95 (m, 1H), 7.91-7.56 (m, 1H), 7.72-7.55 (m, 7H), 7.47-7.39 (m, 2H), 7.37-7.31 (m, 1H), 5.26-5.12 (m, 1H), 4.71-4.60 (m, 2H), 4.57-4.48 (m, 1H), 4.23-4.11 (m, 1H), 3.99-3.90 (m, 1H), 3.88-3.67 (m, 2H), 3.63-3.49 (m, 1H), 2.39-2.35 (m, 1H), 2.30-2.16 (m, 4H), |
| | phosphonic | | | 2.15-1.78 (m, 4H) |
| | acid | | | |
| 197 | ((2- (((5S,8S,10a R)-3-acetyl- 6-oxo-8-(7- phenyl-3,4- dihydro-2H- benzo[b][1,4] oxazine-4- carbonyl) decahydro- pyrrolo[1,2- a][1,5] diazocin-5- yl)carbamoyl) benzo[b]thio- phen-5-yl) | 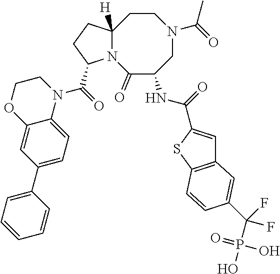 [SMILES: CC(=O)N1CC[C@H]2CC[C@@H](C(=O)N3CCOc4cc(-c5ccccc5)ccc43)N2C(=O)[C@@H](NC(=O)c2cc3cc(C(F)(F)P(=O)(O)O)ccc3s2)C1] | 753.5 [M + H]⁺ | ¹H NMR (400 MHz, DMSO-d₆) δ 8.71 (d, J =6.4 Hz, 1H), 8.31 (d, J = 3.6 Hz, 1H), 8.15- 8.03 (m, 2H), 7.67- 7.56 (m, 3H), 7.48- 7.38 (m, 2H), 7.38- 7.28(m, 1H), 7.25- 7.12 (m, 2H), 5.07- 4.87 (m, 2H), 4.44- 4.24 (m, 4H), 3.80- 3.64 (m, 3H), 3.52- 3.38 (m, 4H), 2.45- 2.35 (m, 2H), 2.13 (s, 3H), 2.07-1.75 (m, |
| | difluoromethyl) | | | 4H) |
| | phosphonic | | | |
| | acid | | | |
| 198 | ((2- (((5S,8S,10a R)-3-acetyl- 8-((3-(2- (methylamino)- 2- oxoethyl)- [1,1′- biphenyl]-4- yl)carbamoyl)- 6- oxodeca- hydrpyrrolo[1,2- a][1,5] diazocin-5- yl)carbamoyl) benzo[b]thio- | 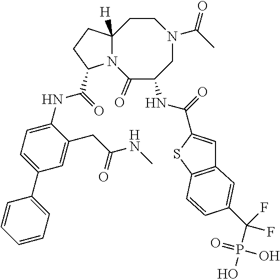 [SMILES: CNC(=O)Cc1cc(-c2ccccc2)ccc1NC(=O)[C@@H]1CC[C@@H]2CCN(C(C)=O)C[C@H](NC(=O)c3cc4cc(C(F)(F)P(=O)(O)O)ccc4s3)C(=O)N21] | 782.6 [M + H]⁺ | ¹H NMR (400 MHz, METHANOL-d₄) δ 8.19-8.11 (m, 2H), 8.05-7.98 (m, 1H), 7.71-7.65 (m, 1H), 7.64-7.58 (m, 3H), 7.58-7.50 (m, 2H), 7.46-7.40 (m, 2H), 7.36-7.29 (m, 1H), 5.23-5.10 (m, 1H), 4.70-4.63 (m, 1H), 4.53-4.38 (m, 1H), 4.01-3.90 (m, 1H), 3.84-3.73 (m, 2H), 3.71-3.58 (m, 2H), 3.57-3.49 (m, 1H), 2.74-2.66 (m, 3H), |
| | phen-5-yl) | | | 2.51-2.40 (m, 1H), |
| | difluoromethyl) | | | 2.29-2.14 (m, 5H), |
| | phosphonic | | | 2.11-1.98 (m, 2H), |
| | acid | | | 1.97-1.87 (m, 1H) |
| 199 | ((7- (((5S,8S,10a R)-3-acetyl- 8-(cinnolin- 6- ylcarbamoyl)- 6-oxodeca- hydropyrrolo [1,2- a][1,5]diazocin- 5- yl)carbamoyl) naphthalen- 2- yl)difluorom ethyl) phosphonic acid | 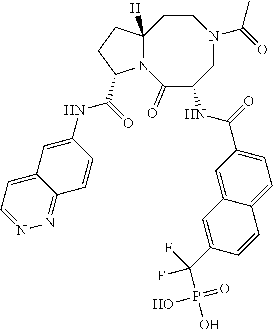 [SMILES: CC(=O)N1CC[C@H]2CC[C@@H](C(=O)Nc3ccc4nnccc4c3)N2C(=O)[C@@H](NC(=O)c2ccc3ccc(C(F)(F)P(=O)(O)O)cc3c2)C1] | 681.2 [M + H]⁺ | ¹H NMR (400 MHz, DMSO-d₆) δ 10.86- 10.78 (m, 1H), 9.28- 9.19 (m, 1H), 8.67- 8.64 (m, 1H), 8.58 (d, J = 7.2 Hz, 1H), 8.53- 8.46 (m, 1H), 8.41 (d, J = 9.2 Hz, 1H), 8.21 (s, 1H), 8.18 (d, J = 6.0 Hz, 1H), 8.11-8.03 (m, 3H), 7.91 (dd, J = 2.4, 9.2 Hz, 1H), 7.72 (d, J = 8.4 Hz, 1H), 5.17-5.05 (m, 1H), 5.03-4.86 (m, 1H), 4.62-4.56 (m, 1H), 4.46-4.25 (m, 2H), 3.95-3.86 (m, 2H), 2.20 (s, 3H), 2.15 (s, |
| | | | | 2H), 2.01-1.92 (m, |
| | | | | 2H), 1.89-1.76 (m, |
| | | | | 2H) |
| 200 | ((7- (((5S,8S,10a R)-3-acetyl- 8-((S)-2- benzyl- pyrrolidine-1- carbonyl)-6- oxodeca- hydropyrrolo[1,2- a][1,5] diazocin-5- yl) carbamoyl) naphthalen- 2-yl) difluoromethyl) phosphonic acid | 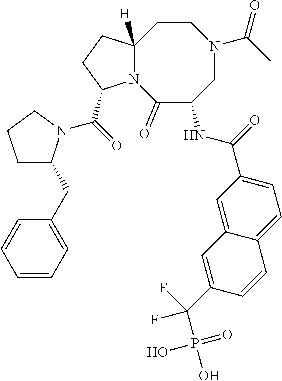 [SMILES: CC(=O)N1CC[C@H]2CC[C@@H](C(=O)N3CCC[C@H]3Cc3ccccc3)N2C(=O)[C@@H](NC(=O)c2ccc3ccc(C(F)(F)P(=O)(O)O)cc3c2)C1] | 697.4 [M + H]⁺ | ¹H NMR (400 MHz, METHANOL-d₄) δ 8.54 (s, 1H), 8.33-8.17 (m, 1H), 8.10-7.96 (m, 3H), 7.81 (d, J = 8.0 Hz, 1H), 7.36-7.14 (m, 5H), 5.12 (m, 1H), 4.74 (s, 1H), 4.40-4.28 (m, 2H), 4.17-4.04 (m, 1H), 4.00-3.90 (m, 1H), 3.87-3.79 (m, 1H), 3.59-3.46 (m, 3H), 3.11-3.01 (m, 1H), 2.71-2.60 (m, 1H), 2.40-2.37 (m, 2H), 2.32-2.15 (m, 3H), 2.10-1.77 (m, 8H) |
| 201 | ((2- (((5S,8S,10a R)-3-acetyl- 8-((3- (difluoromethyl) phenyl) (methyl) carbamoyl)-6- oxodeca- hydropyrrolo[1,2- a][1,5] diazocin-5- yl)carbamoyl) benzo[b]thio- phen-5- yl)difluoromethyl) phosphonic acid | 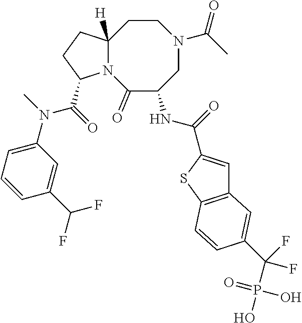 [SMILES: CC(=O)N1CC[C@H]2CC[C@@H](C(=O)N(C)c3cccc(C(F)F)c3)N2C(=O)[C@@H](NC(=O)c2cc3cc(C(F)(F)P(=O)(O)O)ccc3s2)C1] | 699.3 [M + H]⁺ | ¹H NMR (400 MHz, METHANOL-d₄) δ 8.23-8.07 (m, 2H), 7.97-7.86 (m, 1H), 7.80-7.71 (m, 1H), 7.68-7.49 (m, 4H), 6.98-6.54 (m, 1H), 5.10-4.92 (m, 1H), 4.84-4.75 (m, 3H), 4.41-4.23 (m, 2H), 3.99 (dd, J = 3.6, 14.2 Hz, 1H), 3.30-3.19 (m, 3H), 2.33-2.22 (m, 3H), 2.08-1.69 (m, 6H) |
| 202 | ((2- (((5S,8S,10a R)-3-acetyl- 8-((4- (difluoromethyl) phenyl) (methyl) carbamoyl)-6- oxodeca- hydropyrrolo [1,2-a][1,5] diazocin-5- yl)carbamoyl) benzo[b]thio- phen-5-yl) difluoromethyl) phosphonic acid | 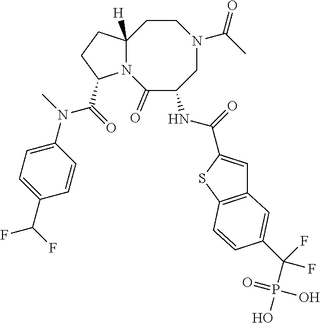 [SMILES: CC(=O)N1CC[C@H]2CC[C@@H](C(=O)N(C)c3ccc(C(F)F)cc3)N2C(=O)[C@@H](NC(=O)c2cc3cc(C(F)(F)P(=O)(O)O)ccc3s2)C1] | 699.3 [M + H]⁺ | ¹H NMR (400 MHz, METHANOL-d₄) δ 8.20 (s, 1H), 8.16-8.08 (m, 1H), 7.99-7.89 (m, 1H), 7.78-7.70 (m, 1H), 7.69-7.52 (m, 4H), 7.00-6.58 (m, 1H), 5.16-4.99 (m, 1H), 4.99-4.90 (m, 1H), 4.47-4.20 (m, 2H), 3.99-3.91 (m, 1H), 3.90-3.53 (m, 3H), 3.29-3.24 (m, 2H), 2.43-2.16 (m, 3H), 2.07-1.89 (m, 5H), 1.82-1.66 (m, 1H) |
| 203 | ((7- (((5S,8S,10a R)-3-acetyl- 8-(rel-trans)- 3-cyano-4- phenyl- pyrrolidine- 1- carbonyl)-6- oxodeca- hydropyrrolo [1,2- a][1,5] diazocin-5- yl)carbamoyl) naphthalen-2-yl) difluoromethyl) | 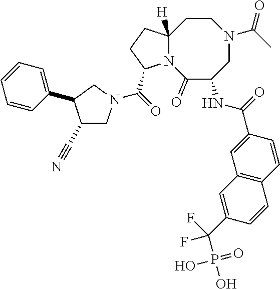 [SMILES: CC(=O)N1CC[C@H]2CC[C@@H](C(=O)N3C[C@H](c4ccccc4)[C@@H](C#N)C3)N2C(=O)[C@@H](NC(=O)c2ccc3ccc(C(F)(F)P(=O)(O)O)cc3c2)C1] | 708.3 [M + H]⁺ | ¹H NMR (400 MHz, METHANOL-d₄) δ 8.67-8.48 (m, 1H), 8.28 (s, 1H), 8.12-7.94 (m, 3H), 7.83 (d, J = 8.8 Hz, 1H), 7.55-7.29 (m, 5H), 5.20-5.12 (m, 1H), 4.60-4.32 (m, 2H), 4.26-3.99 (m, 3H), 3.92-3.76 (m, 3H), 3.71-3.55 (m, 4H), 2.39-2.28 (m, 3H), 2.26 (d, J = 7.2 Hz, 2H), 2.11-1.88 (m, 4H) |
| | phosphonic | | | |
| | acid | | | |
| 204 | ((2- (((5S,8S,10a R)-3-acetyl- 8-((rel- trans)-3- cyano-2- phenyl- azetidine-1- carbonyl)-6- oxodeca- hydropyrrolo [1,2-a][1,5] diazocin-5- yl)carbamoyl) benzo[b]thio- phen-5-yl) difluoromethyl) | 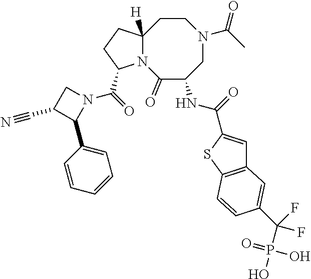 [SMILES: CC(=O)N1CC[C@H]2CC[C@@H](C(=O)N3C[C@@H](C#N)[C@@H]3c3ccccc3)N2C(=O)[C@@H](NC(=O)c2cc3cc(C(F)(F)P(=O)(O)O)ccc3s2)C1] | 700.04 [M + H]⁺ | ¹H NMR (400 MHz, METHANOL-d₄) δ 8.26-8.19 (m, 1H), 8.18-8.08 (m, 1H), 7.97-7.89 (m, 1H), 7.83-7.78 (m, 1H), 7.55-7.32 (m, 5H), 5.16-5.02 (m, 1H), 4.58-4.46 (m, 1H), 4.45-4.32 (m, 1H), 4.32-4.17 (m, 1H), 4.17-4.03 (m, 1H), 4.01-3.85 (m, 1H), 3.83-3.70 (m, 1H), 3.68-3.43 (m, 3H), 2.41-2.29 (m, 1H), 2.28-2.20 (m, 3H), |
| | phosphonic | | | 2.19-2.06 (m, 2H), |
| | acid | | | 2.07-1.93 (m, 2H), |
| | | | | 1.93-1.71 (m, 2H) |
| 205 | ((2- (((5S,8S,10a R)-3-acetyl- 8-((rel-cis)- 4-cyano-2- phenyl- pyrrolidine-1- carbonyl)-6- oxodeca- hydropyrrolo [1,2-a][1,5] diazocin-5- yl)carbamoyl) benzo[b]thio- phen-5-yl) difluoromethyl) phosphonic | 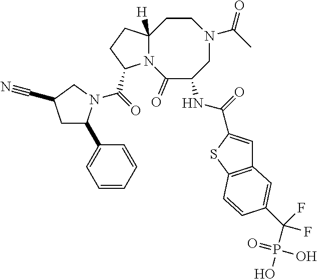 [SMILES: CC(=O)N1CC[C@H]2CC[C@@H](C(=O)N3C[C@H](C#N)C[C@@H]3c3ccccc3)N2C(=O)[C@@H](NC(=O)c2cc3cc(C(F)(F)P(=O)(O)O)ccc3s2)C1] | 714.3 [M + H]⁺ | ¹H NMR (400 MHz, METHANOL-d₄) δ 8.21-8.11 (m, 2H), 8.05-7.97 (m, 1H), 7.73 (d, J = 8.8 Hz, 1H), 7.42-7.30 (m, 5H), 5.49-5.42 (m, 1H), 5.17-4.97 (m, 1H), 4.81-4.77 (m, 1H), 4.40-4.30 (m, 2H), 4.28-4.18 (m, 1H), 4.00-3.64 (m, 4H), 3.61-3.48 (m, 2H), 2.25-2.21 (m, 3H), 2.20-2.11 (m, 1H), 2.09-1.98 (m, 1H), 1.94-1.82 (m, |
| | acid | | | 2H), 1.82-1.57 (m, |
| | | | | 3H) |
| 206 | ((2- (((5S,8S,10a R)-3-acetyl- 8-((rel- trans)-4- cyano-2- phenyl- pyrrolidine-1- carbonyl)-6- oxodeca- hydropyrrolo[1,2- a][1,5]diazocin- 5- yl)carbamoyl) benzo[b]thio- phen-5-yl) difluoromethyl) | 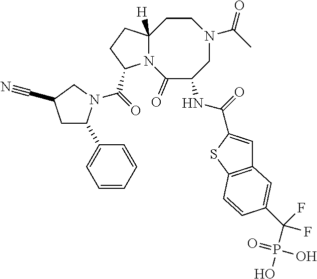 [SMILES: CC(=O)N1CC[C@H]2CC[C@@H](C(=O)N3C[C@H](C#N)C[C@H]3c3ccccc3)N2C(=O)[C@@H](NC(=O)c2cc3cc(C(F)(F)P(=O)(O)O)ccc3s2)C1] | 714.3 [M + H]⁺ | ¹H NMR (400 MHz, METHANOL-d₄) δ 8.21-7.88 (m, 3H), 7.73-7.64 (m, 1H), 7.44-7.28 (m, 3H), 7.26-7.15 (m, 2H), 5.38-5.24 (m, 1H), 5.14-4.93 (m, 1H), 4.83-4.78 (m, 1H), 4.46-4.19 (m, 3H), 4.03-3.95 (m, 1H), 3.95-3.79 (m, 1H), 3.80-3.61 (m, 1H), 3.55-3.37 (m, 2H), 2.70-2.44 (m, 2H), 2.40-2.31 (m, 1H), 2.30-2.21 (m, 3H), |
| | phosphonic | | | 2.19 (s, 1H), 2.07-1.94 |
| | acid | | | (m, 2H), 1.93-1.78 (m, |
| | | | | 2H) |
| 207 | ((2- (((5S,8S,10a R)-3-acetyl- 8-((rel- trans)-3- cyano-2- phenyl- pyrrolidine-1- carbonyl)-6- oxodeca- hydropyrrolo[1,2- a][1,5] diazocin-5- yl)carbamoyl) benzo[b]thio- phen-5-yl) difluoromethyl) | 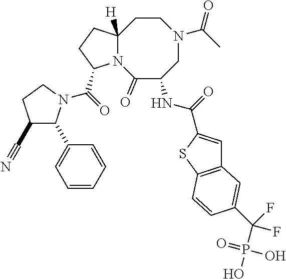 [SMILES: CC(=O)N1CC[C@H]2CC[C@@H](C(=O)N3CC[C@H](C#N)[C@H]3c3ccccc3)N2C(=O)[C@@H](NC(=O)c2cc3cc(C(F)(F)P(=O)(O)O)ccc3s2)C1] | 714.1 [M + H]⁺ | ¹H NMR (400 MHz, METHANOL-d₄) δ 8.26-8.17 (m, 1H), 8.16-8.07 (m, 1H), 7.99-7.88 (m, 1H), 7.77 (d, J = 8.0 Hz, 1H), 7.50-7.21 (m, 5H), 5.77-5.27 (m, 1H), 5.17-5.00 (m, 1H), 4.46-4.16 (m, 2H), 4.13-3.84 (m, 2H), 3.84-3.61 (m, 2H), 3.59-3.33 (m, 2H), 3.30-3.15 (m, 1H), 2.42 (s, 1H), 2.39- 2.12 (m, 5H), 2.10- 1.66 (m, 5H) |
| | phosphonic | | | |
| | acid | | | |
| 208 | ((2- (((5S,8S,10a R)-3-acetyl- 8-(3-(3- cyanophenyl) azetidine-1- carbonyl)-6- oxodeca- hydropyrrolo[1,2- a][1,5]diazocin- 5-yl) carbamoyl) benzo[b]thio- phen-5-yl) difluoromethyl) | 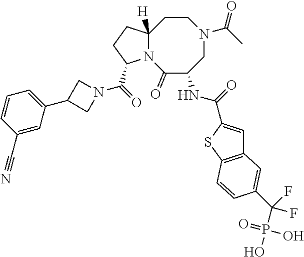 [SMILES: CC(=O)N1CC[C@H]2CC[C@@H](C(=O)N3CC(c4cccc(C#N)c4)C3)N2C(=O)[C@@H](NC(=O)c2cc3cc(C(F)(F)P(=O)(O)O)ccc3s2)C1] | 700.1 [M + H]⁺ | ¹H NMR (400 MHz, DMSO-d₆) δ 8.39- 8.28 (m, 1H), 8.25- 8.09 (m, 2H), 7.94- 7.81 (m, 1H), 7.80- 7.69 (m, 2H), 7.66- 7.55 (m, 2H), 5.02 (s, 1H), 4.95-4.87 (m, 1H), 4.83 (s, 1H), 4.64 (d, J = 8.4 Hz, 1H), 4.45-4.40 (m, 2H), 4.31-4.25 (m, 2H), 3.99 (dd, J = 6.8, 14.8 Hz, 1H), 3.92-3.82 (m, 2H), 3.69 (dd, J = |
| | phosphonic | | | 4.0, 7.2 Hz, 2H), 3.40- |
| | acid | | | 3.29 (m, 1H), 2.24 (s, |
| | | | | 1H), 2.19-2.09 |
| | | | | (m,3H), 2.07-1.72 (m, |
| | | | | 5H) |

### Prepared According to General Scheme 3: Phosphonic Acids

The following compounds in Table 16 were prepared according to the representative procedure described above for the synthesis of (difluoro(2-(((5S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonic acid (3) utilizing the appropriate starting materials and modifications.

TABLE 16

| Comp- | | | | |
|---|---|---|---|---|
| ound | Name | Structure | LCMS | NMR |
| 209 | ((7- (((5S,8S,10a R)-2- benzhydryl- 8-((rel- trans)-3- cyano-4- phenyl- pyrrolidine-1- carbonyl)-6- oxodeca- hydropyrrolo [1,2-a][1,4] diazocin-5- yl)carbamoyl) naphthalen- 2-yl) difluoromethyl) phosphonic acid | 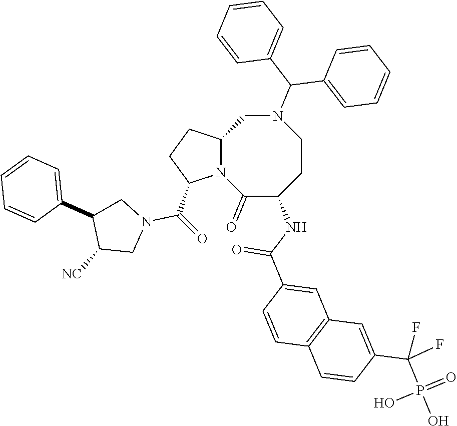 [SMILES: N#C[C@H]1CN(C(=O)[C@@H]2CC[C@@H]3CN(C(c4ccccc4)c4ccccc4)CC[C@H](NC(=O)c4ccc5ccc(C(F)(F)P(=O)(O)O)cc5c4)C(=O)N32)C[C@@H]1c1ccccc1] | 832.3 [M + H]⁺ | ¹H NMR (400 MHz, DMSO-d₆) δ 9.28- 8.51 (m, 2H), 8.32- 7.89 (m, 4H), 7.85- 7.16 (m, 16H), 5.62- 5.42 (m, 1H), 5.00- 3.45 (m, 11H), 2.99- 2.64 (m, 2H), 2.40- 1.90 (m, 3H), 1.84- 1.43 (m, 3H). |
| 210 | ((2- (((5S,8S,10a R)-2- benzhydryl- 6-oxo-8-(3- (pyridin-3- yl)azetidine- 1-carbonyl) decahydro- pyrrolo [1,2-a][1,4] diazocin- 5- yl)carbamoyl) benzo[b]thio- phen-5-yl) difluoromethyl) phosphonic acid | 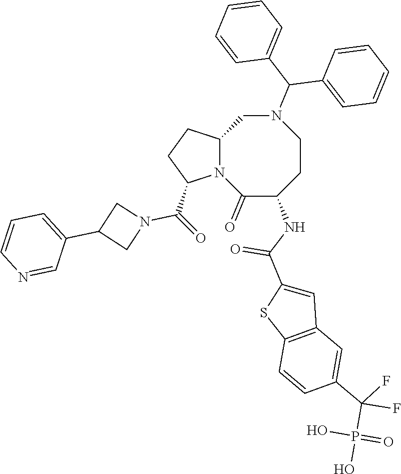 [SMILES: O=C(N[C@H]1CCN(C(c2ccccc2)c2ccccc2)C[C@H]2CC[C@@H](C(=O)N3CC(c4cccnc4)C3)N2C1=O)c1cc2cc(C(F)(F)P(=O)(O)O)ccc2s1] | 800.2 [M + H]⁺ | ¹H NMR (400 MHz, DMSO-d₆) δ 9.04- 8.85 (m, 1H), 8.69- 8.25 (m, 3H), 8.21- 8.00 (m, 2H), 7.98 - 7.77 (m, 1H), 7.59 (d, J = 8.6 Hz, 1H), 7.52- 7.39 (m, 5H), 7.36- 7.17 (m, 6H), 5.50- 5.36 (m, 1H), 5.03- 4.74 (m, 2H), 4.66- 4.49 (m, 1H), 4.47- 4.13 (m, 5H), 3.99- 3.73 (m, 3H), 3.27- 3.11 (m, 1H), 2.14- 1.94 (m, 2H), 1.84- 1.65 (m, 2H), 1.61- 1.37 (m, 2H). |
| 211 | ((7- (((5S,8S,10a R)-8-((rel- trans)-3- cyano-4- phenylpyrrolidine- 1-carbonyl)-6- oxodeca- hydropyrrolo [1,2- a][1,4]diazocin- 5- yl)carbamoyl) naphthalen-2- yl)difluorom ethyl)phosphonic acid | 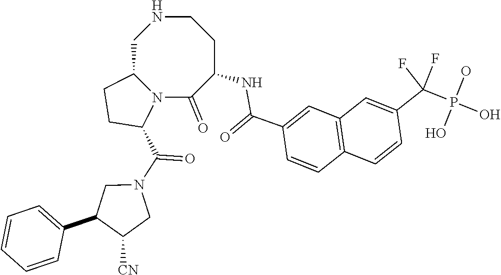 | 666.2 [M + H]⁺ | ¹H NMR (400 MHz, DMSO-d₆) δ 9.40- 9.30 (m, 1H), 9.09- 8.80 (m, 1H), 8.72- 8.57 (m, 1H), 8.28- 8.16 (m, 1H), 8.08- 7.91 (m, 3H), 7.77- 7.68 (m, 1H), 7.45- 7.29 (m, 5H), 5.38- 5.10 (m, 1H), 4.86- 4.64 (m, 2H), 4.32- 3.86 (m, 4H), 3.70- 3.62 (m, 2H), 3.53- 3.44 (m, 2H), 3.23- 3.07 (m, 2H), 2.43- 2.25 (m, 2H), 2.14- 2.00 (m, 2H), 1.91- |
| | | | | 1.65 (m, 2H). |
| 212 | (difluoro(2- (((5S,8S,10a R)-6-oxo-8- (3-(pyridin- 3- yl)azetidine- 1- carbonyl) decahydropyrrolo [1,2- a][1,4]diazocin- 5- yl)carbamoyl) benzo[b]thio- phen-5- yl)methyl) phosphonic acid | 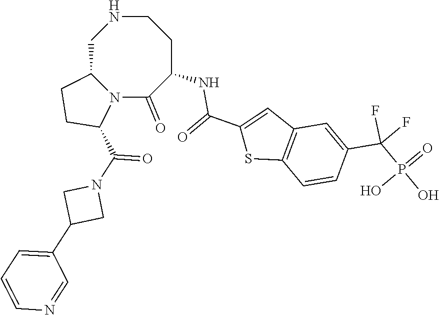 [SMILES: O=C(N[C@H]1CCNC[C@H]2CC[C@@H](C(=O)N3CC(c4cccnc4)C3)N2C1=O)c1cc2cc(C(F)(F)P(=O)(O)O)ccc2s1] | 634.1 [M + H]⁺ | ¹H NMR (400 MHz, DMSO-d₆) δ 9.23- 9.08 (m, 2H), 8.72- 8.43 (m, 2H), 8.32 (s, 1H), 8.17-8.06 (m, 2H), 8.05-7.84 (m, 1H), 7.64-7.41 (m, 2H), 5.24-5.08 (m, 1H), 4.87-4.59 (m, 2H), 4.58-4.47 (m, 1H), 4.45-4.23 (m, 2H), 4.02-3.91 (m, 2H), 3.47-3.33 (m, 2H), 3.32-3.23 (m, 1H), 3.19-3.09 (m, 1H), 2.39-2.23 (m, 2H), 2.22-2.04 (m, 2H), 2.02-1.72 (m, 2H). |
| 213 | ((7- (((5S,8S,10a R)-2- ((benzyloxy) carbonyl)-8- ((rel-trans)- 3-cyano-4- phenylpyrrolidine- 1- carbonyl)-6- oxodeca- hydropyrrolo [1,2- a][1,4]diazocin-5- yl)carbamoyl) naphthalen- 2-yl) difluoromethyl) phosphonic acid | 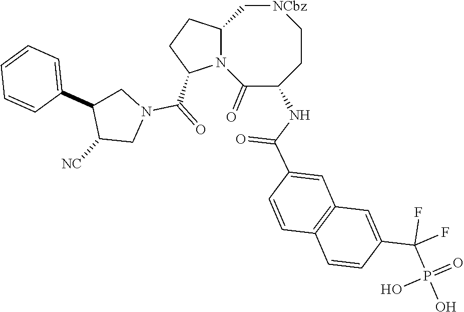 [SMILES: N#C[C@H]1CN(C(=O)[C@@H]2CC[C@@H]3CN(C(=O)OCc4ccccc4)CC[C@H](NC(=O)c4ccc5ccc(C(F)(F)P(=O)(O)O)cc5c4)C(=O)N32)C[C@@H]1c1ccccc1] | 800 [M + H]⁺ | ¹H NMR (400 MHz, DMSO-d₆) δ 8.96- 8.76 (m, 1H), 8.69- 8.58 (m, 1H), 8.20 (s, 1H), 8.15-7.94 (m, 3H), 7.73 (d, J = 8.6 Hz, 1H), 7.51-7.19 (m, 10H), 5.30-5.05 (m, 2H), 4.85-4.65 (m, 2H), 4.38-4.27 (m, 2H), 4.09 3.96 (m, 4H), 3.80-3.67 (m, 4H), 3.35-3.25 (m, 1H), 2.40-1.66 (m, 6H). (TFA salt) |
| 214 | ((2- (((5S,8S,10a R)-2- ((benzyloxy) carbonyl)-6- oxo-8-(3- (pyridin-3- yl)azetidine- 1- carbonyl) decahydropyrrolo [1,2- a][1,4]diazocin- 5- yl)carbamoyl) benzo[b]thio- phen-5- yl)difluoromethyl) phosphonic acid | 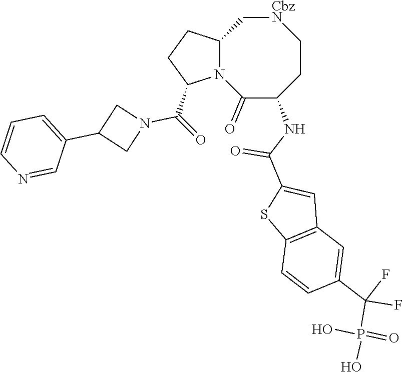 [SMILES: O=C(N[C@H]1CCN(C(=O)OCc2ccccc2)C[C@H]2CC[C@@H](C(=O)N3CC(c4cccnc4)C3)N2C1=O)c1cc2cc(C(F)(F)P(=O)(O)O)ccc2s1] | 768.2 [M + H]⁺ | ¹H NMR (400 MHz, DMSO-d₆) δ 9.24- 8.95 (m, 1H), 8.60 (s, 1H), 8.55-8.43 (m, 1H), 8.39-8.24 (m, 1H), 8.21-8.03 (m, 2H), 8.01-7.82 (m, 1H), 7.60 (d, J = 8.3 Hz, 1H), 7.51-7.42 (m, 1H), 7.41-7.18 (m, 5H), 5.31-5.00 (m, 2H), 4.89-4.58 (m, 2H), 4.53-4.23 (m, 4H), 4.10-3.95 (m, 3H), 3.61-3.49 (m, 2H), 3.34-3.20 (m, 1H), 2.38-2.06 (m, 3H), 1.97-1.77 (m, 3H). |
| 215 | ((2- (((5S,8S,10a R)-8-([1,1′- biphenyl]-4- ylcarbamoyl)- 6-oxo-3-(3- phenylpropanoyl) decahydropyrrolo [1,2a][1,5]diazocin- 5- yl)carbamoyl)- 1H-indol-5- yl)difluorom ethyl)phosphonic acid | 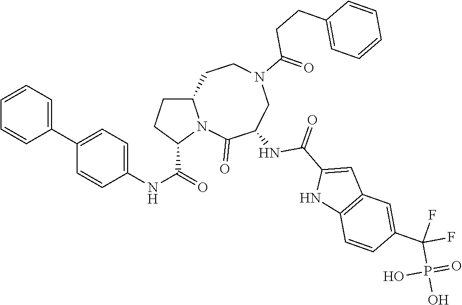 [SMILES: O=C(N[C@H]1CN(C(=O)CCc2ccccc2)CC[C@H]2CC[C@@H](C(=O)Nc3ccc(-c4ccccc4)cc3)N2C1=O)c1cc2cc(C(F)(F)P(=O)(O)O)ccc2[nH]1] | 784.3 [M + H]⁺ | ¹H NMR (400 MHz, DMSO-d₆) δ 11.84- 11.69 (m, 1H), 10.33- 10.18 (m, 1H), 8.85- 8.34 (m, 1H), 7.83- 7.15 (m, 18H), 5.12- 4.88 (m, 1H), 4.59 - 4.41 (m, 1H), 4.38 - 4.10 (m, 1H), 4.09 - 3.93 (m, 1H), 3.83- 3.69 (m, 1H), 3.53- 3.43 (m, 1H), 2.98- 2.56 (m, 4H), 2.31- 2.14 (m, 1H), 2.10- 1.83 (m, 3H), 1.82- 1.64 (m, 2H), 1.48- 1.41 (m, 1H). |
| 216 | (difluoro(2- (((5S,8S,10a R)-8- (methyl(phenyl) carbamoyl) -6-oxo-3- ((S)-2- phenylpropanoyl) decahydro- pyrrolo[1,2 a][1,5]diazocin- 5- yl)carbamoyl)- 1H-indol-5- yl)methyl) phosphonic acid | 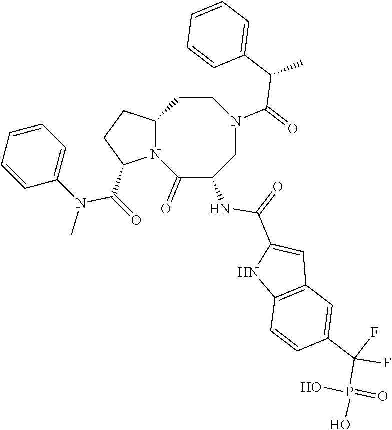 [SMILES: C[C@H](C(=O)N1CC[C@H]2CC[C@@H](C(=O)N(C)c3ccccc3)N2C(=O)[C@@H](NC(=O)c2cc3cc(C(F)(F)P(=O)(O)O)ccc3[nH]2)C1)c1ccccc1] | 722.1 [M + H]⁺ | ¹H NMR (400 MHz, DMSO-d₆) δ 11.87- 11.71 (m, 1H), 8.73- 8.47 (m, 1H), 7.89- 7.78 (m, 1H), 7.54- 7.16 (m, 13H), 4.91- 4.66 (m, 1H), 4.50- 4.07 (m, 4H), 3.99- 3.79 (m, 3H), 3.15 (s, 3H), 2.02-1.54 (m, 6H), 1.44-1.27 (m, 3H). |
| 217 | (difluoro(2- (((5S,8S,10a R)-8- (methyl (phenyl) carbamoyl)- 6-oxo-3- ((R)-2- phenylpropanoyl) decahydropyrrolo [1,2 a][1,5]diazocin- 5- yl)carbamoyl)- 1H-indol-5- yl)methyl) phosphonic acid | 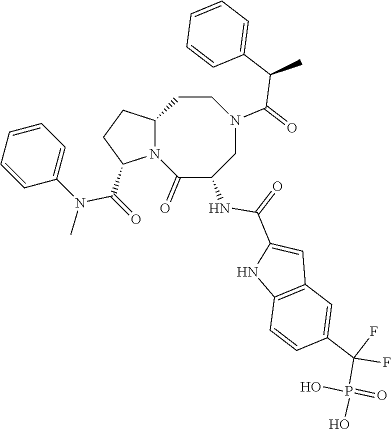 [SMILES: C[C@@H](C(=O)N1CC[C@H]2CC[C@@H](C(=O)N(C)c3ccccc3)N2C(=O)[C@@H](NC(=O)c2cc3cc(C(F)(F)P(=O)(O)O)ccc3[nH]2)C1)c1ccccc1] | 722.1 [M + H]⁺ | ¹H NMR (400 MHz, DMSO-d₆) δ 12.11 (s, 1H), 8.32-8.11 (m, 1H), 7.86 (s, 1H), 7.56- 7.16 (m, 13H), 5.03 - 4.91 (m, 1H), 4.74- 4.63 (m, 1H), 4.32- 4.15 (m, 2H), 4.00- 3.90 (m, 2H), 3.24- 3.11 (m, 4H), 2.98- 2.84 (m, 1H), 2.23- 2.09 (m, 1H), 1.96- 1.77 (m, 3H), 1.70- 1.50 (m, 2H), 1.40 (d, J = 4.0 Hz , 3H). |
| 218 | ((2- (((5S,8S,10a R)-3-(2,2- difluoroethyl)- 8- (methyl(phenyl) carbamoyl)-6- oxodeca- hydropyrrolo [1,2- a][1,5]diazocin- 5-yl)carbamoyl)- 1H-indol-5- yl) difluoromethyl) phosphonic acid | 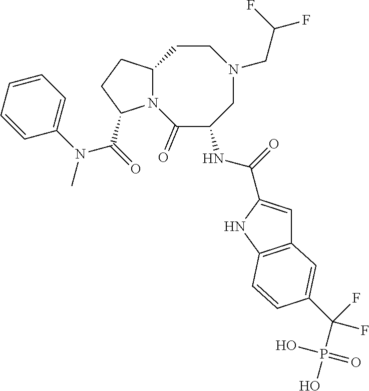 [SMILES: CN(C(=O)[C@@H]1CC[C@@H]2CCN(CC(F)F)C[C@H](NC(=O)c3cc4cc(C(F)(F)P(=O)(O)O)ccc4[nH]3)C(=O)N21)c1ccccc1] | 654.2 [M + H]⁺ | ¹H NMR (400 MHz, DMSO-d₆) δ 11.86 (s, 1H), 8.33 (s, 1H), 7.83 (s, 1H), 7.50-7.26 (m, 8H), 6.17 (t, J = 55.5 Hz, 1H), 4.97-4.85 (m, 1H), 4.28-4.22 (m, 2H), 3.23-2.99 (m, 8H), 1.95-1.62 (m, 6H). |
| 219 | (difluoro(2- (((5S,8S,10a R)-8- (methyl (phenyl) carbamoyl)- 6-oxo-3- (2,2,2- trifluoroethyl) decahydropyrrolo [1,2- a][1,5]diazocin-5- yl)carbamoyl)- 1H-indol-5- yl)methyl) phosphonic acid | 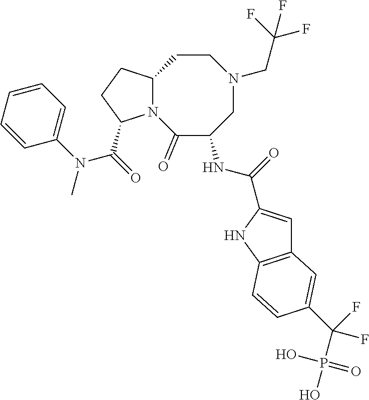 [SMILES: CN(C(=O)[C@@H]1CC[C@@H]2CCN(CC(F)(F)F)C[C@H](NC(=O)c3cc4cc(C(F)(F)P(=O)(O)O)ccc4[nH]3)C(=O)N21)c1ccccc1] | 672.2 [M + H]⁺ | ¹H NMR (400 MHz, DMSO-d₆) δ 11.84 (s, 1H), 8.29 (d, J = 7.4 Hz, 1H), 7.83 (s, 1H), 7.51-7.30 (m, 8H), 4.89-4.83 (m, 1H), 4.27-4.19 (m, 2H), 3.60-3.53 (m, 2H), 3.33-3.20 (m, 2H), 3.17 (s, 3H), 3.11- 2.98 (m, 2H), 1.92- 1.64 (m, 6H). |
| 220 | ((2- (((5S,8S,10a R)-3- benzoyl-8- (methyl(phenyl) carbamoyl)-6- oxodeca- hydropyrrolo [1,2- a][1,5]diazocin-5- yl)carbamoyl)- 1H-indol-5- yl)difluoromethyl) phosphonic acid | 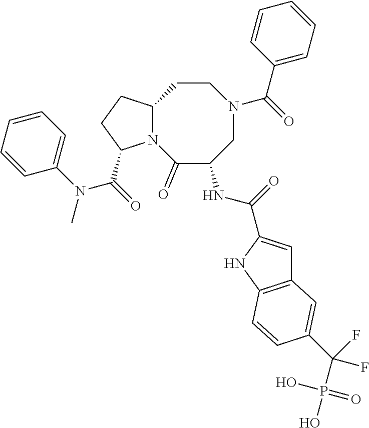 [SMILES: CN(C(=O)[C@@H]1CC[C@@H]2CCN(C(=O)c3ccccc3)C[C@H](NC(=O)c3cc4cc(C(F)(F)P(=O)(O)O)ccc4[nH]3)C(=O)N21)c1ccccc1] | 694.2 [M + H]⁺ | ¹H NMR (400 MHz, DMSO-d₆) δ 11.81- 11.67 (m, 1H), 8.80- 8.49 (m, 1H), 7.88- 7.76 (m, 1H), 7.57- 7.30 (m, 13H), 5.26- 4.98 (m, 1H), 4.47- 4.35 (m, 1H), 4.24- 4.18 (m, 1H), 3.94- 3.87 (m, 1H), 3.66 - 3.57 (m, 3H), 3.20 (s, 3H), 2.23-1.74 (m, 6H). |
| 221 | (difluoro(2- (((5S,8S,10a R)-3- isobutyryl-8- (methyl(phenyl) carbamoyl)-6- oxodeca- hydropyrrolo [1,2- a][1,5]diazocin-5- yl)carbamoyl)- 1H-indol-5- yl)methyl) phosphonic acid | 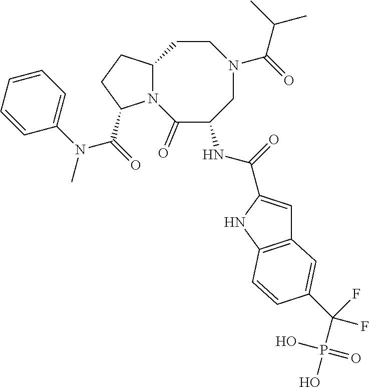 [SMILES: CC(C)C(=O)N1CC[C@H]2CC[C@@H](C(=O)N(C)c3ccccc3)N2C(=O)[C@@H](NC(=O)c2cc3cc(C(F)(F)P(=O)(O)O)ccc3[nH]2)C1] | 660.1 [M + H]⁺ | ¹H NMR (400 MHz, DMSO-d₆) δ 12.06 - 11.68 (m, 1H), 8.69- 8.32 (m, 1H), 7.85 (s, 1H), 7.51-7.32 (m, 8H), 5.00-4.70 (m, 1H), 4.29-3.75 (m, 5H), 3.18-3.13 (m, 4H), 2.03-1.51 (m, 7H), 1.15-1.03 (m, 6H). |

### Prepared According to General Scheme 4: Phosphonic Acids

The following compounds in Table 17 were prepared according to the representative procedure described above for the synthesis of ((7-(((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamoyl)naphthalen-2-yl)difluoromethyl)phosphonic acid (4) and utilizing the appropriate starting materials and modifications.

TABLE 17

| Com- | | | | |
|---|---|---|---|---|
| pound | Name | Structure | LCMS | NMR |
| 222 | ((2-(((5S, 8S,10aR)- 8-((3S,4R or 3R,4S)-3- cyano-4- phenyl- pyrrolidine- 1- carbonyl)-3- ethyl-6-oxo- decahydro- pyrrolo[1,2- a][1,5] diazocin-5- yl) carbamoyl) benzo[b] thiophen-5- yl)difluoro- methyl) phosphonic | 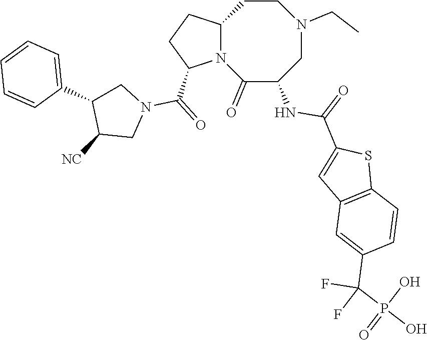 [SMILES: CCN1CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3s2)C1] | 700.2 [M + H]⁺ | ¹H NMR (400 MHz, METHANOL-d₄) δ 8.18 (s, 1H), 8.07 (d, J = 2.9 Hz, 1H), 7.90 (d, J = 8.6 Hz, 1H), 7.77 (d, J = 8.3 Hz, 1H), 7.46-7.39 (m, 4H), 7.39-7.31 (m, 1H), 5.63-5.53 (m, 1H), 5.12-5.02 (m, 1H), 4.79-4.72 (m, 1H), 4.44-4.29 (m, 1H), 4.19-4.03 (m, 1H), 3.97-3.37 (m, 10H), 2.73-2.52 (m, 1H), 2.41-2.18 (m, 2H), 2.09-1.90 (m, 3H), 1.52-1.44 (m, 3H). |
| | acid | or | | |
| | | 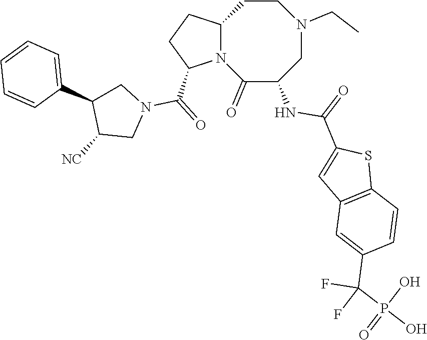 [SMILES: CCN1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(O)O)ccc3s2)C1] | | |
| | | SFC peak 2 was used for | | |
| | | biological testing | | |
| 223 | ((2-(((5S, 8S,10aR)- 8-(dimethyl- carbamoyl)- 6-oxo-3- phenyldeca- hydropyrrolo [1,2-a][1,5] diazocin-5- yl) carbamoyl)- 1H-indol-5- yl)difluoro- methyl) phosphonic acid | 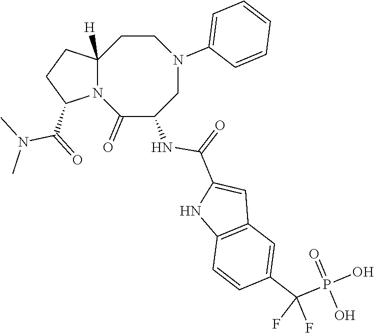 [SMILES: CN(C)C(=O)[C@@H]1CC[C@@H]2CCN(c3ccccc3)C[C@H](NC(=O)c3cc4cc(C(F)(F)P(=O)(O)O)ccc4[nH]3)C(=O)N21] | 604.2 [M + H]⁺ | ¹H NMR (400 MHz, METHANOL-d₄) δ 7.94 (s, 1H), 7.59-7.44 (m, 2H), 7.33-7.21 (m, 3H), 7.16-7.02 (m, 2H), 6.77-6.64 (m, 1H), 5.16-5.06 (m, 1H), 4.93 (t, J = 9.0 Hz, 1H), 4.16-3.95 (m, 3H), 3.82-3.67 (m, 1H), 3.51 (dd, J = 14.8, 10.4 Hz, 1H), 3.19 (s, 3H), 2.99 (s, 3H), 2.48- 2.36 (m, 1H), 2.36- 2.23 (m, 1H), 2.13- 1.99 (m, 1H), 1.99- 1.85 (m, 1H), 1.82- 1.70 (m, 2H). |
| 224 | ((2-(((5S, 8S,10aR)- 8- (dimethyl- carbamoyl)- 3-isobutyl- 6-oxo- decahydro- pyrrolo[1,2- a][1,5] diazocin-5- yl) carbamoyl)- 1H-indol-5- yl)difluoro- methyl) phosphonic acid | 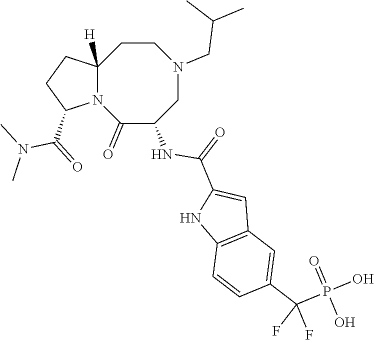 [SMILES: CC(C)CN1CC[C@H]2CC[C@@H](C(=O)N(C)C)N2C(=O)[C@@H](NC(=O)c2cc3cc(C(F)(F)P(=O)(O)O)ccc3[nH]2)C1] | 584.3 [M + H]⁺ | ¹H NMR (400 MHz, METHANOL-d₄) δ 7.98 (s, 1H), 7.58 (d, J = 9.0 Hz, 1H), 7.44 (d, J = 8.8 Hz, 1H), 7.24 (s, 1H), 5.71-5.60 (m, 1H), 5.18-5.07 (m, 1H), 4.79-4.67 (m, 1H), 4.01-3.86 (m, 1H), 3.74-3.52 (m, 3H), 3.27-3.11 (m, 5H), 2.98 (s, 3H), 2.68- 2.54 (m, 1H), 2.46- 2.13 (m, 3H), 2.02- 1.84 (m, 3H), 1.20- 1.03 (m, 6H). |
| 225 | ((2-(((5S, 8S,10aR)- 3-benzyl- 8- (dimethyl- carbamoyl)- 6-oxo- decahydro- pyrrolo[1,2- a][1,5] diazocin-5- yl) carbamoyl)- 1H-indol-5- yl)difluoro- methyl) phosphonic acid | 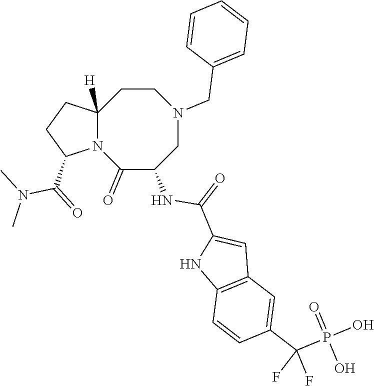 [SMILES: CN(C)C(=O)[C@@H]1CC[C@@H]2CCN(Cc3ccccc3)C[C@H](NC(=O)c3cc4cc(C(F)(F)P(=O)(O)O)ccc4[nH]3)C(=O)N21] | 618.2 [M + H]⁺ | ¹H NMR (400 MHz, METHANOL-d₄) δ 7.97 (s, 1H), 7.72-7.70 (m, 2H), 7.58-7.56 (m, 1H), 7.50-7.40 (m, 4H), 7.19 (s, 1H), 5.57 (br. s., 1H), 5.04 (br. s., 1H), 4.69-4.59 (m, 2H), 4.48-4.45 (m, 1H), 3.88-3.56 (m, 3H), 3.42-3.36 (m, 1H), 3.20-2.98 (m, 6H), 2.54 (br. s., 1H), 2.29-2.05 (m, 2H), 1.89-1.75 (m, 3H). |
| 226 | ((2-(((5S, 8S,10aR)- 2-ethyl-6- oxo-8-(3- (pyridin-3- yl)azetidine- 1-carbonyl) decahydro- pyrrolo[1,2- a][1,4] diazocin-5- yl) carbamoyl) benzo[b] thiophen-5- yl)difluoro- methyl) phosphonic acid | 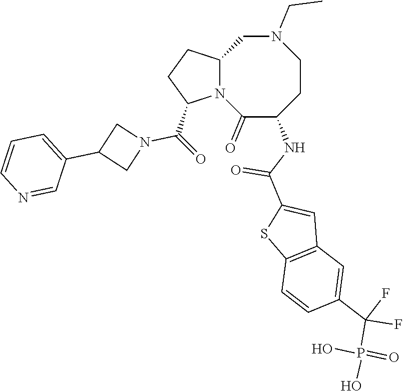 [SMILES: CCN1CC[C@H](NC(=O)c2cc3cc(C(F)(F)P(=O)(O)O)ccc3s2)C(=O)N2[C@H](CC[C@H]2C(=O)N2CC(c3cccnc3)C2)C1] | 661.6 [M + H]⁺ | ¹H NMR (400 MHz, DMSO-d₆) δ 8.59- 8.14 (m, 3H), 8.14- 7.12 (m, 6H), 5.30- 5.13 (m, 1H), 4.96- 4.19 (m, 6H), 4.02- 3.86 (m, 3H), 3.26- 3.11 (m, 4H), 2.36- 2.18 (m, 3H), 2.05- 1.75 (m, 3H), 1.35- 1.26 (m, 3H). (TFA salt) |
| 227 | ((7-(((5S, 8S,10aR)- 8-((rel- trans)-3- cyano-4- phenyl- pyrrolidine- 1-carbonyl)- 2-ethyl-6- oxo- decahydro- pyrrolo[1,2- a][1,4] diazocin-5- yl) carbamoyl) naphthalen- 2- yl)difluoro- methyl) phosphonic acid | 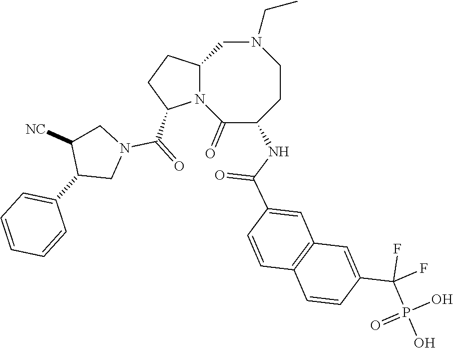 [SMILES: CCN1CC[C@H](NC(=O)c2ccc3ccc(C(F)(F)P(=O)(O)O)cc3c2)C(=O)N2[C@H](CC[C@H]2C(=O)N2C[C@H](c3ccccc3)[C@@H](C#N)C2)C1] | 694 [M + H]⁺ | ¹H NMR (400 MHz, DMSO-d₆) δ 9.03- 8.75 (m, 2H), 8.19- 7.88 (m, 4H), 7.66 (d, J = 8.4 Hz, 1H), 7.47- 7.27 (m, 5H), 5.58- 5.31 (m, 1H), 5.07- 4.66 (m, 2H), 4.35- 3.48 (m, 12H), 2.32- 1.68 (m, 5H), 1.36- 1.19 (m, 4H). (TFA salt) |
| 228 | ((7-(((5S, 8S,10aR)- 8-((rel- trans)-3- cyano-4- phenyl- pyrrolidine- 1- carbonyl)-2- cyclobutyl- 6-oxo- decahydro- pyrrolo[1,2- a][1,4] diazocin-5- yl) carbamoyl) naphthalen- 2- yl)difluoro- methyl) phosphonic acid | 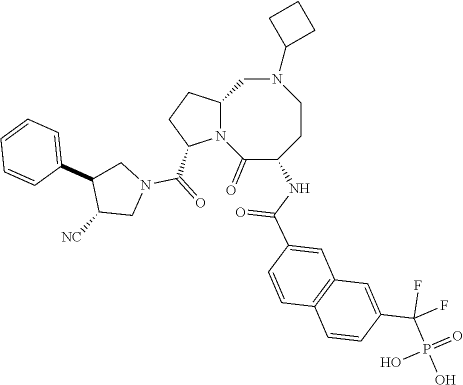 [SMILES: N#C[C@H]1CN(C(=O)[C@@H]2CC[C@@H]3CN(C4CCC4)CC[C@H](NC(=O)c4ccc5ccc(C(F)(F)P(=O)(O)O)cc5c4)C(=O)N32)C[C@@H]1c1ccccc1] | 720.0 [M + H]⁺ | ¹H NMR (400 MHz, DMSO-d₆) δ 8.85-8.83 (m, 2H), 8.22-8.10 (m, 1H), 8.11-7.86 (m, 3H), 7.70 (d, J = 8.4 Hz, 1H), 7.49- 7.24 (m, 5H), 5.53- 5.21 (m, 1H), 4.98- 4.64 (m, 2H), 4.37- 3.59 (m, 9H), 3.32- 3.15 (m, 2H), 2.46- 1.18 (m, 12H). (TFA salt) |
| 229 | (difluoro(7- (((5S,8S, 10aR)-8- (((R)-1-(4- methoxy- phenyl)-4- (methyl- amino)-4- oxobutan-2- yl) carbamoyl)- 3-(5- methyl-1H- indazole-3- carbonyl)- 6-oxo- decahydro- pyrrolo[1,2- a][1,5] diazocin-5- yl) carbamoyl) naphthalen- 2- | 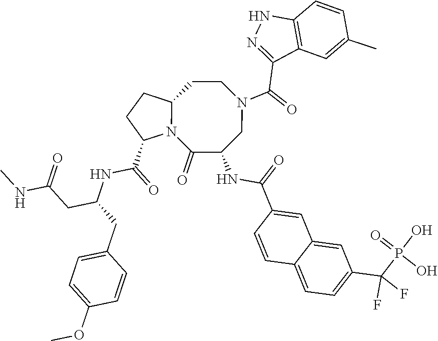 [SMILES: CNC(=O)C[C@@H](Cc1ccc(OC)cc1)NC(=O)[C@@H]1CC[C@@H]2CCN(C(=O)c3n[nH]c4ccc(C)cc34)C[C@H](NC(=O)c3ccc4ccc(C(F)(F)P(=O)(O)O)cc4c3)C(=O)N21] | 874.3 [M + H]⁺ | ¹H NMR (400 MHz, DMSO-d₆) δ 13.68- 13.14 (m, 1H), 9.05- 8.80 (m, 1H), 8.67- 8.45 (m, 1H), 8.24- 8.16 (m, 1H), 8.14- 8.02 (m, 4H), 7.95- 7.85 (m, 1H), 7.75- 7.71 (m, 1H), 7.63- 7.58 (m, 1H), 7.54- 7.38 (m, 1H), 7.31- 7.19 (m, 1H), 7.11- 6.99 (m, 2H), 6.82- 6.74 (m, 2H), 5.22- 5.08 (m, 1H), 4.82- 4.75 (m, 1H), 4.33- 4.18 (m, 5H), 3.84- 3.81 (m, 1H), 3.68- 3.65 (m, 3H), 3.55- 3.47 (m, 2H), 2.73- 2.65 (m, 1H), 2.62- 2.57 (m, 1H), 2.55- 2.52 (m, 3H), 2.46- 2.36 (m, 3H), 2.19- |
| | yl)methyl) | | | 2.05 (m, 4H), 1.88- |
| | phosphonic | | | 1.81 (m, 1H), 1.79- |
| | acid | | | 1.73 (m, 1H). |
| 230 | ((2-(((5S, 8S,10aR)- 8-(dimethyl- carbamoyl)- 3-(5-methyl- 1H-indazole- 3-carbonyl)- 6-oxo- decahydro- pyrrolo[1,2- a][1,5] diazocin-5- yl) carbamoyl) benzo[b] thiophen-5- yl)methyl) phosphonic acid | 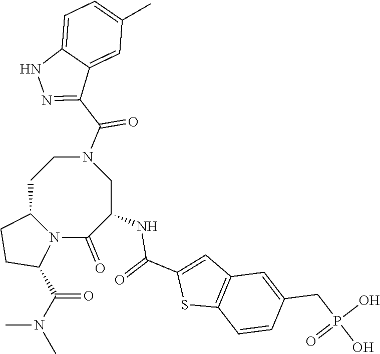 [SMILES: Cc1ccc2[nH]nc(C(=O)N3CC[C@H]4CC[C@@H](C(=O)N(C)C)N4C(=O)[C@@H](NC(=O)c4cc5cc(CP(=O)(O)O)ccc5s4)C3)c2c1] | 667.1 [M + H]⁺ | ¹H NMR (400 MHz, DMSO-d₆) δ 9.01- 8.79 (m, 1H), 8.19- 8.07 (m, 1H), 7.98- 7.77 (m, 2H), 7.77- 7.66 (m, 1H), 7.53- 7.39 (m, 1H), 7.38- 7.14 (m, 2H), 5.18- 5.04 (m, 1H), 4.84- 4.73 (m, 2H), 4.28- 4.15 (m, 2H), 3.61- 3.57 (m, 1H), 3.35- 3.30 (m, 1H), 3.05 (s, 3H), 2.94-2.72 (m, 5H), 2.47-2.33 (m, 3H), 2.31-1.90 (m, 2H), 1.91-1.56 (m, 3H), 1.09-1.00 (m, 1H). |
| 231 | ((2-(((5S, 8S,10aR)- 8- (dimethyl- carbamoyl)- 3-(5-methyl- 1H-indazole- 3-carbonyl)- 6-oxo- decahydro- pyrrolo[1,2- a][1,5] diazocin-5- yl) carbamoyl) benzo[b] thiophen-5- yl)difluoro- methyl) phosphonic acid | 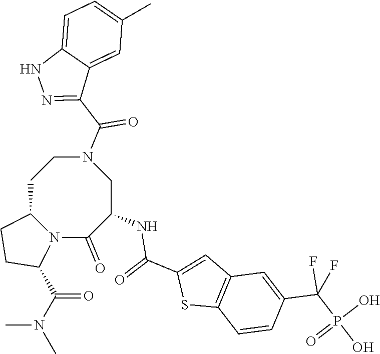 [SMILES: Cc1ccc2[nH]nc(C(=O)N3CC[C@H]4CC[C@@H](C(=O)N(C)C)N4C(=O)[C@@H](NC(=O)c4cc5cc(C(F)(F)P(=O)(O)O)ccc5s4)C3)c2c1] | 703.1 [M + H]⁺ | ¹H NMR (400 MHz, DMSO-d₆) δ 13.53- 13.30 (m, 1H), 9.11- 8.88 (m, 1H), 8.34- 8.22 (m, 1H), 8.16- 8.03 (m, 2H), 7.96- 7.83 (m, 1H), 7.63- 7.55 (m, 1H), 7.53- 7.39 (m, 1H), 7.28- 7.20 (m, 1H), 5.42- 5.07 (m, 1H), 4.85- 4.70 (m, 2H), 4.40- 4.22 (m, 1H), 4.21- 4.13 (m, 1H), 3.66- 3.55 (m, 2H), 3.05 (s, 3H), 2.81 (s, 3H), 2.37-2.24 (m, 1H), 2.14-1.95 (m, 2H), 1.87-1.63 (m, 3H), 1.27-1.22 (m, 3H). |
| 232 | ((R or S)-(2- (((5S,8S, 10aR)-8- ((3S,4R or 3R,4R)-3- cyano-4- phenyl- pyrrolidine- 1-carbonyl)- 3-ethyl- 6-oxo- decahydro- pyrrolo[1,2- a][1,5] diazocin-5- yl) carbamoyl) benzo[b] | 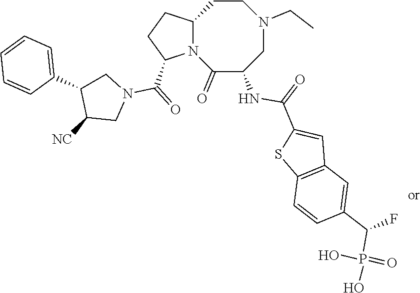 [SMILES: CCN1CC[C@H]2CC[C@@H](C(=O)N3C[C@@H](C#N)[C@H](c4ccccc4)C3)N2C(=O)[C@@H](NC(=O)c2cc3cc([C@H](F)P(=O)(O)O)ccc3s2)C1] | 682.1 [M + H]⁺ | ¹H NMR (400 MHz, DMSO-d₆) δ 8.92 (s, 1H), 8.32-8.07 (m, 1H), 8.03-7.86 (m, 2H), 7.58-7.19 (m, 6H), 5.56 (dd, J = 45.1, 7.6 Hz, 1H), 5.26- 4.93 (m, 1H), 4.77- 4.44 (m, 2H), 4.21- 4.06 (m, 1H), 4.05- 3.91 (m, 2H), 3.86- 3.78 (m, 1H), 3.71- 3.54 (m, 3H), 3.38- 3.29 (m, 1H), 3.26- 3.12 (m, 2H), 3.09- 2.93 (m, 2H), 2.45- 2.34 (m, 1H), 2.15- 2.00 (m, 1H), 1.92- |
| | thiophen-5- yl)fluoro- methyl) phosphonic acid | 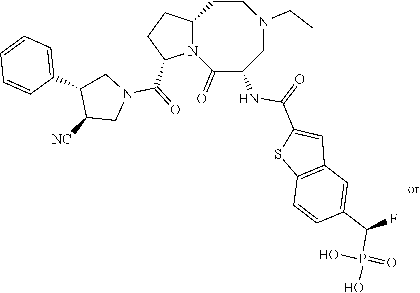 [SMILES: CCN1CC[C@H]2CC[C@@H](C(=O)N3C[C@@H](C#N)[C@H](c4ccccc4)C3)N2C(=O)[C@@H](NC(=O)c2cc3cc([C@@H](F)P(=O)(O)O)ccc3s2)C1] | | 1.60 (m, 4H), 1.29- 1.07 (m, 3H). |
| | | 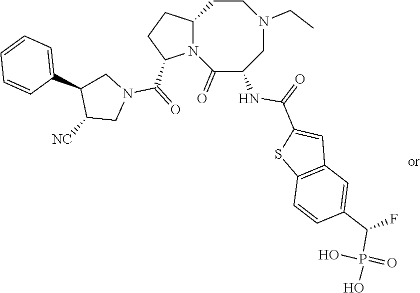 [SMILES: CCN1CC[C@H]2CC[C@@H](C(=O)N3C[C@H](c4ccccc4)[C@@H](C#N)C3)N2C(=O)[C@@H](NC(=O)c2cc3cc([C@H](F)P(=O)(O)O)ccc3s2)C1] | | |
| | | 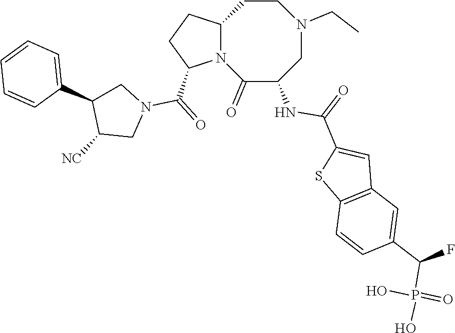 [SMILES: CCN1CC[C@H]2CC[C@@H](C(=O)N3C[C@H](c4ccccc4)[C@@H](C#N)C3)N2C(=O)[C@@H](NC(=O)c2cc3cc([C@@H](F)P(=O)(O)O)ccc3s2)C1] | | |
| | | SFC peak 2 was used for | | |
| | | biological testing, | | |
| | | Benzothiophene peak 1 was | | |
| | | used for synthesis | | |
| 233 | ((7-(((5S, 8S,10aR)- 8-(rel- tran)-3- cyano-4- phenyl- pyrrolidine- 1- carbonyl)-2- (5-methyl- 1H-indazole- 3-carbonyl)- 6-oxo- decahydro- pyrrolo[1,2- a][1,4] diazocin-5- yl) carbamoyl) naphthalen- 2- yl)difluoro- methyl) phosphonic acid | 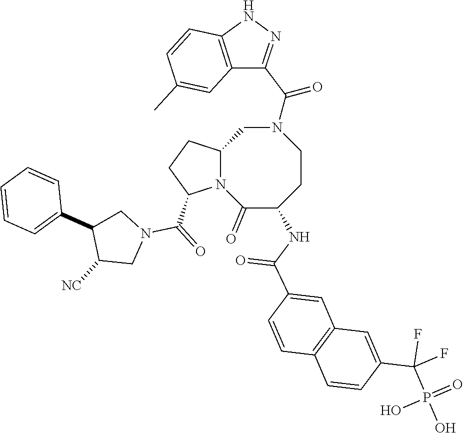 [SMILES: Cc1ccc2[nH]nc(C(=O)N3CC[C@H](NC(=O)c4ccc5ccc(C(F)(F)P(=O)(O)O)cc5c4)C(=O)N4[C@H](CC[C@H]4C(=O)N4C[C@H](c5ccccc5)[C@@H](C#N)C4)C3)c2c1] | 824.0 [M + H]⁺ | ¹H NMR(400 MHz, DMSO-d₆) δ 13.83- 13.30 (m, 1H), 9.01- 8.45 (m, 2H), 8.24- 7.95 (m, 4H), 7.87- 7.65 (m, 2H), 7.55- 7.20 (m, 7H), 5.07- 4.53 (m, 5H), 4.34- 3.65 (m, 8H), 2.76- 2.61 (m, 1H), 2.44- 1.62 (m, 8H). (TFA salt) |
| 234 | ((7-(((5S, 8S,10aR)- 2-(2,2- difluoro- ethyl)- 6-oxo-8-(6- (2-oxo-1,2- dihydro- pyridin-4- yl)-4- azaspiro[2.4] heptane-4- carbonyl) decahydro- pyrrolo[1,2- a][1,4] diazocin-5- yl) carbamoyl) naphthalen- 2- yl)difluoro- methyl) phosphonic | 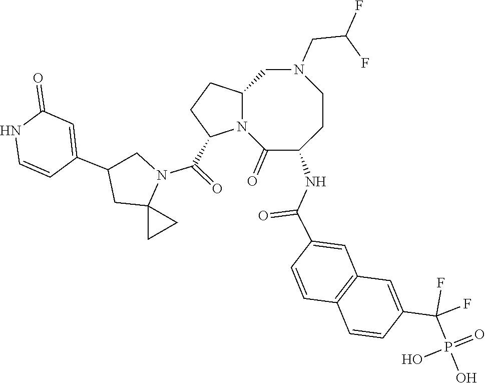 [SMILES: O=C(N[C@H]1CCN(CC(F)F)C[C@H]2CC[C@@H](C(=O)N3CC(c4cc[nH]c(=O)c4)CC34CC4)N2C1=O)c1ccc2ccc(C(F)(F)P(=O)(O)O)cc2c1] | 748.2 [M + H]⁺ | ¹H NMR (400 MHz, DMSO-d₆) δ 8.77 (d, J = 7.3 Hz, 1H), 8.66 (s, 1H), 8.19 (s, 1H), 8.09 (d, J = 8.6 Hz, 1H), 8.06 (s, 2H), 7.72 (d, J = 8.3 Hz, 1H), 7.30 (d, J = 6.9 Hz, 1H), 7.14-6.90 (m, 1H), 6.25 (s, 1H), 6.19 (d, J = 6.9 Hz, 1H), 5.21-5.08 (m, 1H), 4.65-4.56 (m, 1H), 4.34-4.23 (m, 3H), 3.63-3.52 (m, 4H), 3.34-3.24 (m, 2H), 2.92-2.74 (m, 2H), 2.22-2.13 (m, 2H), 2.05-1.91 (m, 5H), 1.74-1.64 (m, 3H), 1.59-1.52 (m, 1H), 0.90-0.81 (m, 1H). (TFA salt) |
| | acid | | | |
| 235 | ((2-(((5S, 8S,10aR)- 3- (imidazo [1,2-a] pyridin-7- ylmethyl)-6- oxo-8-(3- (pyridin-3- yl)azetidine- 1-carbonyl) decahydro- pyrrolo[1,2- a][1,5] diazocin-5- yl) carbamoyl) benzo[b] thiophen-5- yl)methyl) phosphonic acid | 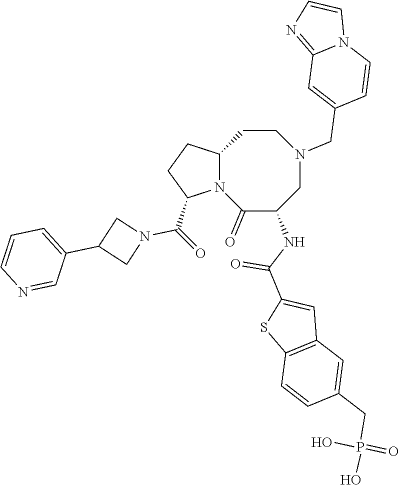 [SMILES: O=C(N[C@H]1CN(Cc2ccn3ccnc3c2)CC[C@H]2CC[C@@H](C(=O)N3CC(c4cccnc4)C3)N2C1=O)c1cc2cc(CP(=O)(O)O)ccc2s1] | 728.3 [M + H]⁺ | ¹H NMR (400 MHz, DMSO-d₆) δ 8.88- 8.79 (m, 1H), 8.76- 8.64 (m, 2H), 8.64- 8.54 (m, 1H), 8.34- 8.24 (m, 1H), 8.17- 8.10 (m, 2H), 8.07- 7.94 (m, 2H), 7.92- 7.85 (m, 1H), 7.83- 7.72 (m, 2H), 7.67- 7.52 (m, 1H), 7.48- 7.33 (m, 1H), 5.18- 5.08 (m, 1H), 4.95- 4.89 (m, 1H), 4.69- 4.64 (m, 1H), 4.53- 4.47 (m, 2H), 4.43- 4.41 (m, 1H), 4.31- 4.28 (m, 1H),4.04- 4.02 (m, 2H), 3.32- 3.22 (m, 1H), 3.17- 2.91 (m, 6H), 2.31- 2.02 (m, 2H), 2.01- 1.73 (m, 3H), 1.72- 1.52 (m, 1H). |
| 236 | ((7-(((5S, 8S,10aR)- 8-((rel- trans)-3- cyano-4- phenyl- pyrrolidine- 1- carbonyl)-3- (5-methyl- 1H-indazole- 3-carbonyl)- 6-oxo- decahydro- pyrrolo[1,2- a][1,5] diazocin-5- yl) carbamoyl) naphthalen- 2-yl)di- fluoro- | 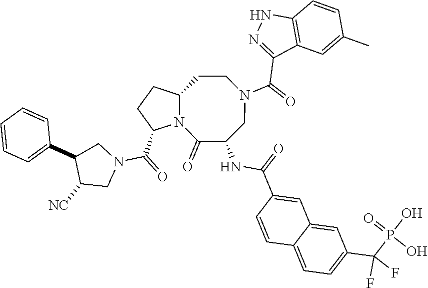 [SMILES: Cc1ccc2[nH]nc(C(=O)N3CC[C@H]4CC[C@@H](C(=O)N5C[C@H](c6ccccc6)[C@@H](C#N)C5)N4C(=O)[C@@H](NC(=O)c4ccc5ccc(C(F)(F)P(=O)(O)O)cc5c4)C3)c2c1] | 824.0 [M + H]⁺ | ¹H NMR (400 MHz, DMSO-d₆) δ 13.73- 12.93 (m, 1H), 9.02- 8.33 (m, 2H), 8.32- 8.00 (m, 4H), 8.00- 7.85 (m, 1H), 7.79- 7.66 (m, 1H), 7.65- 7.15 (m, 6H), 5.30- 5.03 (m, 1H), 4.83- 4.57 (m, 2H), 4.43- 4.35 (m, 1H), 4.29- 4.24 (m, 1H), 4.18- 4.09 (m, 2H), 3.96- 3.85 (m, 3H), 3.71- 3.67 (m, 1H), 3.53- 3.47 (m, 1H), 3.33- 3.13 (m, 1H), 2.56- 2.51 (m, 3H), 2.46- 2.42 (m, 1H), 2.41- 2.31 (m, 1H), 2.18- 1.61 (m, 4H). |
| | methyl) | | | |
| | phosphonic | | | |
| | acid | | | |
| 237 | (difluoro(7- (((5S,8S, 10aR)-3-(5- methyl-1H- indazole-3- carbonyl)-8- (methyl- carbamoyl)- 6-oxo- decahydro- pyrrolo[1,2- a][1,5] diazocin-5- yl) carbamoyl) naphthalen- 2- yl)methyl) phosphonic acid | 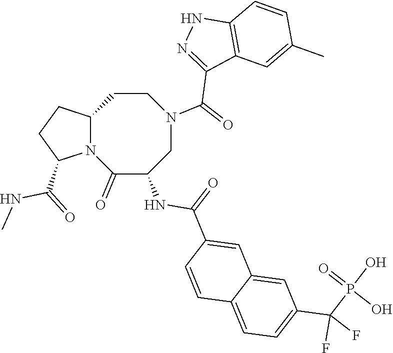 [SMILES: CNC(=O)[C@@H]1CC[C@@H]2CCN(C(=O)c3n[nH]c4ccc(C)cc34)C[C@H](NC(=O)c3ccc4ccc(C(F)(F)P(=O)(O)O)cc4c3)C(=O)N21] | 683.0 [M + H]⁺ | ¹H NMR (400 MHz, DMSO-d₆) δ 13.54- 13.34 (m, 1H), 8.97- 8.73 (m, 1H), 8.72- 8.58 (m, 1H), 8.27- 8.15 (m, 1H), 8.14- 8.00 (m, 3H), 7.87- 7.79 (m, 1H), 7.76- 7.68 (m, 1H), 7.55- 7.39 (m, 1H), 7.30- 7.17 (m, 1H), 5.21- 5.01 (m, 1H), 4.76- 4.66 (m, 1H), 4.35- 4.29 (m, 2H), 4.15- 4.11 (m, 1H), 3.75- 3.69 (m, 1H), 3.57- 3.50 (m, 1H), 2.54 (s, 3H), 2.42 (s, 3H), 2.17-1.87 (m, 4H), 1.82-1.54 (m, 2H). |
| 238 | ((7-(((5S, 8S,10aR)- 8-((3R,4S or 3S,4R)-3- cyano-4-(2- oxo-1,2- dihydro- pyridin-4-yl) pyrrolidine- 1-carbonyl)- 3-ethyl- 6-oxo- decahydro- pyrrolo[1,2- a][1,5] diazocin-5- yl) carbamoyl) naphthalen- 2-yl) difluoro- methyl) | 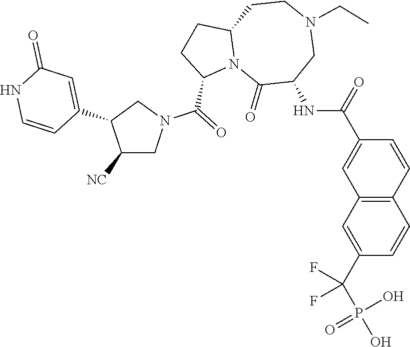 [SMILES: CCN1CC[C@H]2CC[C@@H](C(=O)N3C[C@@H](C#N)[C@H](c4cc[nH]c(=O)c4)C3)N2C(=O)[C@@H](NC(=O)c2ccc3ccc(C(F)(F)P(=O)(O)O)cc3c2)C1] | 711.0 [M + H]⁺ | ¹H NMR (411 MHz, DMSO-d₆) δ 9.04 (s, 1H), 8.66-8.56 (m, 1H), 8.28-8.17 (m, 1H), 8.07-7.95 (m, 3H), 7.80-7.69 (m, 1H), 7.44-7.34 (m, 1H), 6.42-6.34 (m, 1H), 6.31-6.19 (m, 1H), 5.65-5.40 (m, 1H), 4.93-4.68 (m, 2H), 4.22-3.71 (m, 12H), 3.69-3.64 (m, 3H), 2.30-1.59 (m, 6H), 1.40-1.27 (m, 3H). |
| | phosphonic | or | | |
| | acid | 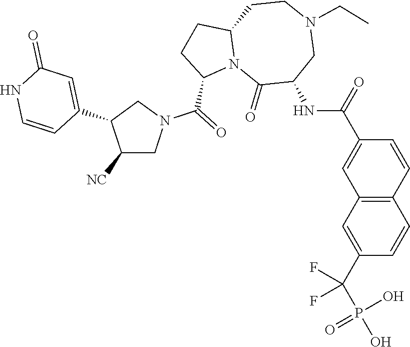 [SMILES: CCN1CC[C@H]2CC[C@@H](C(=O)N3C[C@@H](C#N)[C@H](c4cc[nH]c(=O)c4)C3)N2C(=O)[C@@H](NC(=O)c2ccc3ccc(C(F)(F)P(=O)(O)O)cc3c2)C1] | | |
| | | SFC peak 2 was used for | | |
| | | biological testing | | |
| 239 | (difluoro(7- (((5S,8S, 10aR)-8- (((R)-1-(4- methoxy- phenyl)-4- (methyl- amino)-4- oxobutan-2- yl) carbamoyl)- 3-(5- methyl-1H- indazole-3- carbonyl)- 6-oxo- decahydro- pyrrolo[1,2- a][1,5] diazocin-5- yl) carbamoyl) naphthalen- 2- | 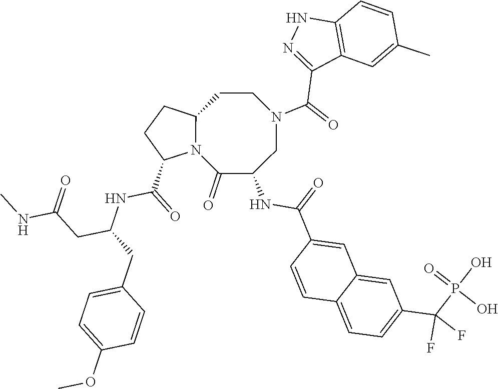 | 999.4 [M + H]⁺ | ¹H NMR (400 MHz, CDCl₃) δ 8.56-8.43 (m, 1H), 8.38-8.19 (m, 1H), 8.08-8.06 (m, 1H), 8.02-7.86 (m, 3H), 7.78-7.68 (m, 1H), 7.40-7.30 (m, 1H), 6.92-6.81 (m, 1H), 6.33-6.15 (m, 1H), 5.16-4.99 (m, 1H), 4.64-4.48 (m, 2H), 4.35-4.11 (m, 6H), 4.03-3.88 (m, 1H), 3.81-3.67 (m, 1H), 3.67-3.56 (m, 1H), 3.43-3.26 (m, 2H), 3.21-3.03 (m, 6H), 2.96-2.81 (m, 4H), 2.44-2.36 (m, 4H), 2.35-2.22 (m, 1H), 2.21-2.05 (m, 4H), 1.96-1.89 (m, 1H), 1.87-1.82 (m, 1H), 1.20-1.08 (m, |
| | yl)methyl) | | | 3H), 0.97-0.88 (m, |
| | phosphonic | | | 12H). |
| | acid | | | |
| 240 | ((7-(((5S, 8S,10aR)- 8-((rel- trans)-3- cyano-4-(2- oxo-1,2- dihydro- pyridin-4-yl) pyrrolidine- 1-carbonyl)- 3-ethyl- 6-oxo- decahydro- pyrrolo[1,2- a][1,5] diazocin-5- yl) carbamoyl) | 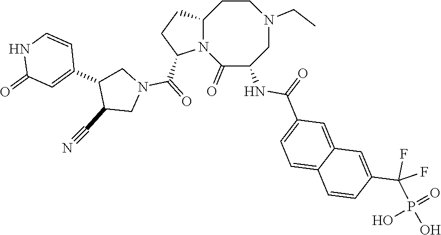 [SMILES: CCN1CC[C@H]2CC[C@@H](C(=O)N3C[C@@H](C#N)[C@H](c4cc[nH]c(=O)c4)C3)N2C(=O)[C@@H](NC(=O)c2ccc3ccc(C(F)(F)P(=O)(O)O)cc3c2)C1] | 711.3 [M + H]⁺ | ¹H NMR (400 MHz, DMSO-d₆) δ 8.70- 8.54 (m, 1H), 8.39- 8.19 (m, 1H), 8.04- 7.92 (m, 3H), 7.82- 7.72 (m, 1H), 7.45- 7.35 (m, 1H), 6.50- 6.34 (m, 1H), 6.31- 6.20 (m, 1H), 5.72- 5.15 (m, 1H), 5.10- 4.62 (m, 2H), 4.35- 3.71 (m, 9H), 3.72- 3.66 (m, 3H), 2.20- 1.98 (m, 2H), 1.94- 1.79 (m, 3H), 1.39- 1.11 (m, 4H). |
| | naphthalen- | | | |
| | 2- | | | |
| | yl)difluoro- | | | |
| | methyl) | | | |
| | phosphonic | | | |
| | acid | | | |
| 241 | ((7-(((5S, 8S,10aR)- 8-((rel- trans)-3- cyano-4- phenyl- piperidine-1- carbonyl)-3- (2,2- difluoro- ethyl)- 6-oxo- decahydro- pyrrolo[1,2- a][1,5] diazocin-5- yl) carbamoyl) naphthalen- | 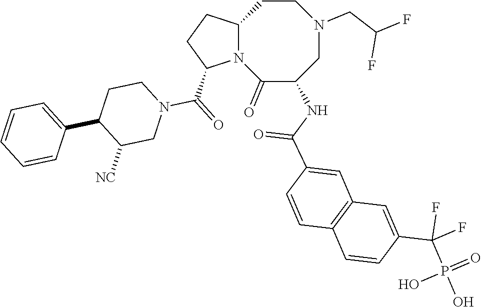 [SMILES: N#C[C@H]1CN(C(=O)[C@@H]2CC[C@@H]3CCN(CC(F)F)C[C@H](NC(=O)c4ccc5ccc(C(F)(F)P(=O)(O)O)cc5c4)C(=O)N32)CC[C@@H]1c1ccccc1] | 744 [M + H]⁺ | ¹H NMR (400 MHz, DMSO-d₆) δ 8.79- 7.87 (m, 6H), 7.82- 7.64 (m, 1H), 7.54- 7.19 (m, 5H), 6.36- 6.08 (m, 1H), 5.09- 4.12 (m, 15H), 2.16- 1.48 (m, 8H). (TFA salt). |
| | 2-yl)di- | | | |
| | fluoro- | | | |
| | methyl) | | | |
| | phosphonic | | | |
| | acid | | | |
| 242 | ((2-(((5S, 8S,10aR)- 3-(2,2- difluoro- ethyl)- 8-((rel- trans)-3- (difluoro- methyl)-4- phenyl- pyrrolidine- 1-carbonyl)- 6-oxo- decahydro- pyrrolo[1,2- a][1,5] diazocin-5- yl) carbamoyl) benzo[b] thiophen-5- yl)difluoro- methyl) phosphonic acid | 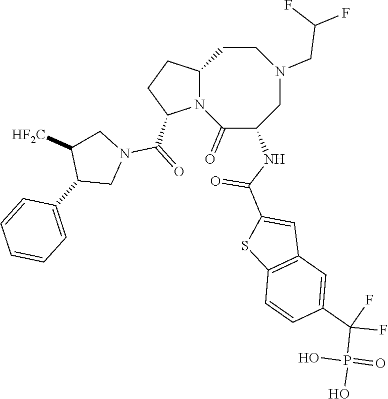 | 761.2 [M + H]⁺ | ¹H NMR (400 MHz, DMSO-d₆) δ 8.68 (s, 1H), 8.31 (d, J = 3.3 Hz, 1H), 8.15 (dd, J = 8.3, 2.9 Hz, 1H), 8.10 (s, 1H), 7.60 (d, J = 8.5 Hz, 1H), 7.61-7.26 (m, 5H), 6.31-5.97 (m, 2H), 4.97-4.94 (m, 1H), 4.70-4.59 (m, 1H), 4.40-4.28 (m, 1H), 4.20-4.02 (m, 1H), 3.95-3.82 (m, 1H), 3.75-3.65 (m, 1H), 3.56-3.42 (m, 1H), 3.31-3.08 (m, 8H), 2.37-2.23 (m, 2H), 1.88-1.71 (m, 4H). |
| 243 | ((7-(((5S, 8S,10aR)- 8-((3S,4R or 3R,4S)-3- cyano-4- phenyl- pyrrolidine- 1-carbonyl)- 3-ethyl- 6-oxo- decahydro- pyrrolo[1,2- a][1,5] diazocin-5- yl) carbamoyl) naphthalen- 2- yl)difluoro- methyl) phosphonic | 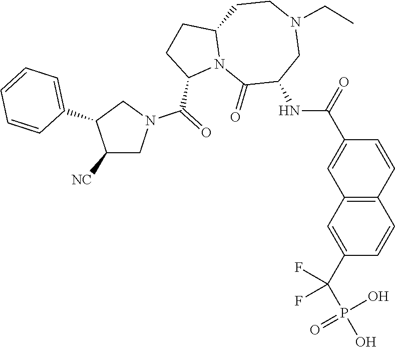 [SMILES: CCN1CC[C@H]2CC[C@@H](C(=O)N3C[C@@H](C#N)[C@H](c4ccccc4)C3)N2C(=O)[C@@H](NC(=O)c2ccc3ccc(C(F)(F)P(=O)(O)O)cc3c2)C1] | 694 [M + H]⁺ | ¹H NMR (400 MHz, DMSO-d₆) δ 9.79- 8.89 (m, 1H), 8.63 (s, 1H), 8.29-7.95 (m, 4H), 7.75 (d, J = 8.5 Hz, 1H), 7.54-7.25 (m, 5H), 5.66-5.48 (m, 1H), 4.96-4.72 (m, 2H), 4.21-3.82 (m, 9H), 3.49-3.35 (m, 3H), 2.22-1.78 (m, 5H), 1.50-1.16 (m, 4H).(TFA salt). |
| | acid | or | | |
| | | 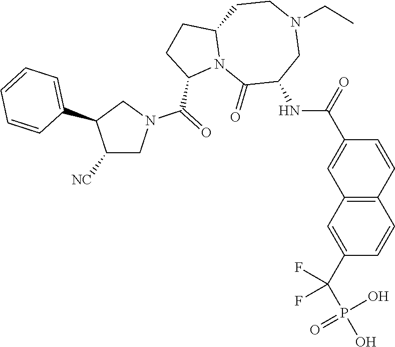 [SMILES: CCN1CC[C@H]2CC[C@@H](C(=O)N3C[C@H](c4ccccc4)[C@@H](C#N)C3)N2C(=O)[C@@H](NC(=O)c2ccc3ccc(C(F)(F)P(=O)(O)O)cc3c2)C1] | | |
| | | SFC peak 2 was used for | | |
| | | biological testing | | |
| 244 | ((7-(((5S, 8S,10aR)- 3-(2,2- difluoro- ethyl)- 8-(methyl(1- (methyl- sulfonyl) piperidin-4- yl) carbamoyl)- 6-oxo- decahydro- pyrrolo[1,2- a][1,5] diazocin-5- yl) carbamoyl) naphthalen- | 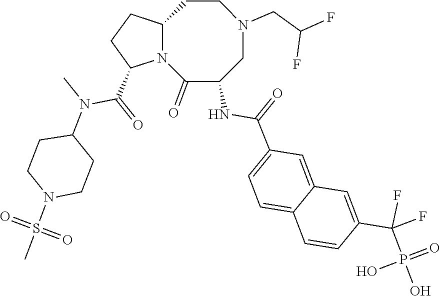 [SMILES: CN(C(=O)[C@@H]1CC[C@@H]2CCN(CC(F)F)C[C@H](NC(=O)c3ccc4ccc(C(F)(F)P(=O)(O)O)cc4c3)C(=O)N21)C1CCN(S(C)(=O)=O)CC1] | 750 [M + H]⁺ | ¹H NMR (400 MHz, DMSO-d₆) δ 8.73- 8.58 (m, 1H), 8.57- 8.30 (m, 1H), 8.21 (s, 1H), 8.13-7.95 (m, 3H), 7.80-7.67 (m, 1H), 6.29-5.96 (m, 1H), 5.02-4.90 (m, 1H), 4.89-4.72 (m, 1H), 4.42-4.31 (m, 2H), 3.68-3.56 (m, 3H), 3.30-3.12 (m, 4H), 3.10-2.99 (m, 2H), 2.93-2.71 (m, 7H), 2.38-2.27 (m, 1H), 2.15-2.04 (m, 1H), 1.94-1.60 (m, 7H), 1.58-1.50 (m, 1H). |
| | 2- | | | |
| | yl)difluoro- | | | |
| | methyl) | | | |
| | phosphonic | | | |
| | acid | | | |
| 245 | ((2-(((5S, 8S,10aR)- 8-((3S,4R or 3R,4S)-3- carbamoyl- 4-phenyl- pyrrolidine- 1-carbonyl)- 3-(2,2- difluoro- ethyl)- 6-oxo- decahydro- pyrrolo[1,2- a][1,5] diazocin-5- yl) carbamoyl) benzo[b] thiophen-5- yl)difluoro- | 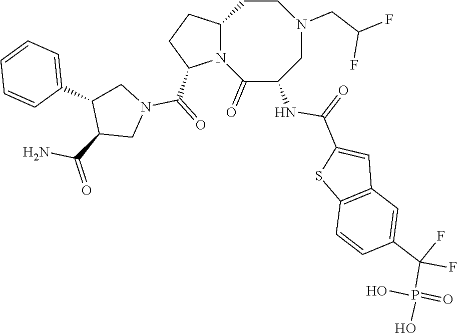 [SMILES: NC(=O)[C@@H]1CN(C(=O)[C@@H]2CC[C@@H]3CCN(CC(F)F)C[C@H](NC(=O)c4cc5cc(C(F)(F)P(=O)(O)O)ccc5s4)C(=O)N32)C[C@H]1c1ccccc1] | 754 [M + H]⁺ | ¹H NMR (400 MHz, DMSO-d₆) δ 8.84- 7.90 (m, 4H), 7.79- 6.84 (m, 8H), 6.31- 5.93 (m, 1H), 4.99- 4.82 (m, 1H), 4.64- 4.50 (m, 1H), 4.40- 4.27 (m, 1H), 3.99- 3.60 (m, 7H), 3.38- 3.12 (m, 5H), 2.38- 1.61 (m, 6H). (TFA salt) |
| | methyl) | | | |
| | phosphonic | or | | |
| | acid | 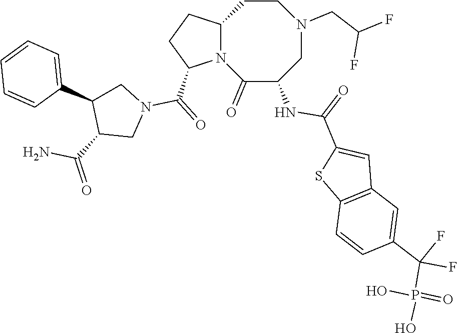 [SMILES: NC(=O)[C@H]1CN(C(=O)[C@@H]2CC[C@@H]3CCN(CC(F)F)C[C@H](NC(=O)c4cc5cc(C(F)(F)P(=O)(O)O)ccc5s4)C(=O)N32)C[C@@H]1c1ccccc1] | | |
| | | SFC peak 2 used for | | |
| | | biological testing | | |
| 246 | ((7-(((5S, 8S,10aR)- 8-((rel- trans)-3- cyano-4- (3,5- difluoro- phenyl) pyrrolidine- 1-carbonyl)- 3-(2,2-di- fluoroethyl)- 6-oxo- decahydro- pyrrolo[1,2- a][1,5] diazocin-5- yl) carbamoyl) | 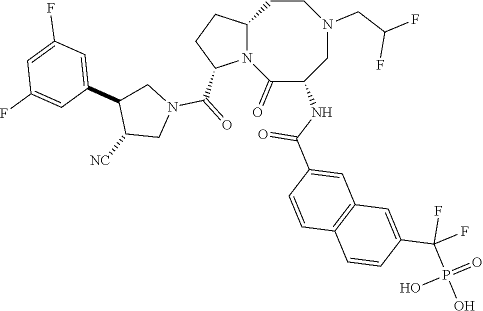 [SMILES: N#C[C@H]1CN(C(=O)[C@@H]2CC[C@@H]3CCN(CC(F)F)C[C@H](NC(=O)c4ccc5ccc(C(F)(F)P(=O)(O)O)cc5c4)C(=O)N32)C[C@@H]1c1cc(F)cc(F)c1] | 766 [M + H]⁺ | ¹H NMR (400 MHz, DMSO-d₆) δ 8.67- 8.56 (m, 1H), 8.55- 8.36 (m, 1H), 8.29- 8.18 (m, 1H), 8.12- 7.92 (m, 3H), 7.73 (d, J = 8.4 Hz, 1H), 7.39- 7.07 (m, 3H), 6.16 (t, J = 55.0 Hz, 1H), 5.06- 4.88 (m, 1H), 4.67- 4.56 (m, 1H), 4.44- 4.37 (m, 1H), 4.32- 4.23 (m, 1H), 4.15- 4.10 (m, 1H), 4.00- 3.93 (m, 2H), 3.79- 3.67 (m, 2H), 3.58- 3.45 (m, 1H), 3.27- 3.04 (m, 5H), 2.36- 2.29 (m, 1H), 2.15- |
| | naphthalen- | | | 2.02 (m, 1H), 1.89- |
| | 2- | | | 1.66 (m, 4H).(TFA |
| | yl)difluoro- | | | salt). |
| | methyl) | | | |
| | phosphonic | | | |
| | acid | | | |
| 247 | ((2-(((5S, 8S,10aR)- 3-(2,2-di- fluoroethyl)- 8-((3S,4R or 3R,4S)-3- (methoxy- carbonyl)-4- phenyl- pyrrolidine- 1-carbonyl)- 6-oxo- decahydro- pyrrolo[1,2- a][1,5] diazocin-5- yl) carbamoyl) benzo[b] thiophen-5- yl)difluoro- | 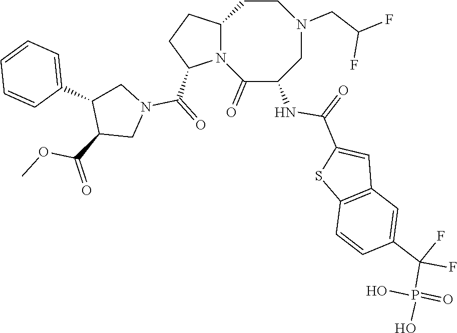 [SMILES: COC(=O)[C@@H]1CN(C(=O)[C@@H]2CC[C@@H]3CCN(CC(F)F)C[C@H](NC(=O)c4cc5cc(C(F)(F)P(=O)(O)O)ccc5s4)C(=O)N32)C[C@H]1c1ccccc1] | 769 [M + H]⁺ | ¹H NMR (400 MHz, DMSO-d₆) δ 8.66- 8.58 (m, 1H), 8.34- 8.28 (m, 1H), 8.16- 8.10 (m, 1H), 8.08 (s, 1H), 7.61-7.56 (m, 1H), 7.34 (s, 3H), 7.29-7.23 (m, 1H), 6.27-5.94 (m, 1H), 4.92-4.85 (m, 1H), 4.62-4.53 (m, 1H), 4.36-4.26 (m, 1H), 4.11-4.04 (m, 1H), 4.01-3.91 (m, 1H), 3.90-3.83 (m, 1H), 3.73-3.63 (m, 2H), 3.56-3.53 (m, 3H), 3.50-3.45 (m, 2H), 3.31-3.25 (m, 2H), 3.16-3.10 (m, 3H), 2.35-2.29 (m, 1H), |
| | methyl) | or | | 2.09-2.00 (m, 1H), |
| | phosphonic acid | 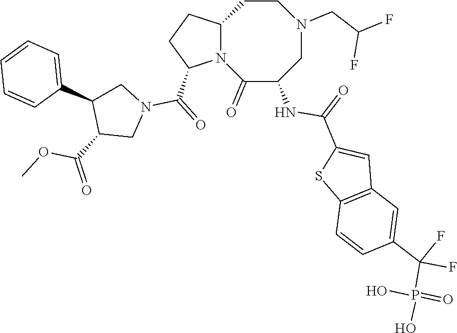 [SMILES: COC(=O)[C@H]1CN(C(=O)[C@@H]2CC[C@@H]3CCN(CC(F)F)C[C@H](NC(=O)c4cc5cc(C(F)(F)P(=O)(O)O)ccc5s4)C(=O)N32)C[C@@H]1c1ccccc1] | | 1.86-1.66 (m, 4H). |
| | | SFC peak 2 was used for | | |
| | | biological testing | | |
| 248 | ((7-(((5S, 8S,10aR)- 8-((3S,4R or 3R,4S)-3- cyano-4- phenyl- pyrrolidine- 1- carbonyl)-3- (2,2-di- fluoroethyl)- 6-oxo- decahydro- pyrrolo[1,2- a][1,5] diazocin-5- yl) carbamoyl) naphthalen- 2- yl)difluoro- methyl) | 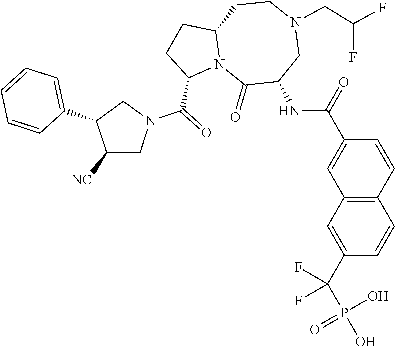 [SMILES: N#C[C@@H]1CN(C(=O)[C@@H]2CC[C@@H]3CCN(CC(F)F)C[C@H](NC(=O)c4ccc5ccc(C(F)(F)P(=O)(O)O)cc5c4)C(=O)N32)C[C@H]1c1ccccc1] | 730 [M + H]⁺ | ¹H NMR (400 MHz, DMSO-d₆) δ 8.70- 8.58 (m, 1H), 8.50- 8.40 (m, 1H), 8.26- 8.18 (m, 1H), 8.14- 7.98 (m, 3H), 7.78- 7.68 (m, 1H), 7.51- 7.30 (m, 5H), 6.33- 5.95 (m, 1H), 5.04- 4.88 (m, 1H), 4.63- 4.55 (m, 1H), 4.41- 4.36 (m, 1H), 4.21- 4.17 (m, 1H), 4.02- 4.01 (m, 1H), 3.85- 3.81 (m, 1H), 3.69- 3.64 (m, 1H), 3.61- 3.44 (m, 3H), 3.36- 3.25 (m, 1H), 3.24- 3.06 (m, 4H), 2.39- 2.30 (m, 1H), 2.17- 1.98 (m, 1H), 1.92- 1.66 (m, 4H). |
| | phosphonic | or | | |
| | acid | 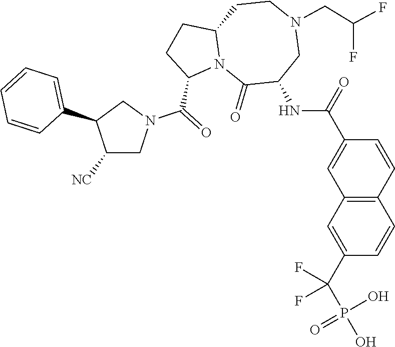 [SMILES: N#C[C@H]1CN(C(=O)[C@@H]2CC[C@@H]3CCN(CC(F)F)C[C@H](NC(=O)c4ccc5ccc(C(F)(F)P(=O)(O)O)cc5c4)C(=O)N32)C[C@@H]1c1ccccc1] | | |
| | | SFC peak 2 was used for | | |
| | | biological testing | | |
| 249 | ((2-(((5S, 8S,10aR)- 8-((3S,4R or 3R,4S)-3- cyano-4- phenyl- pyrrolidine- 1- carbonyl)-3- (2,2-di- fluoroethyl)- 6-oxo- decahydro- pyrrolo[1,2- a][1,5] diazocin-5- yl) carbamoyl) benzo[b] thiophen-5- yl)difluoro- | 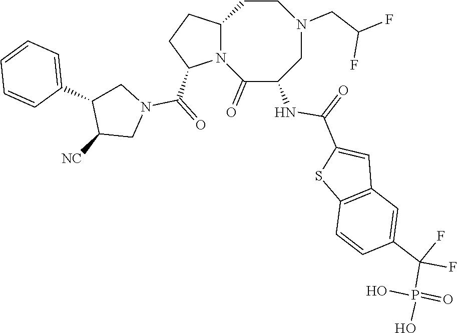 [SMILES: N#C[C@@H]1CN(C(=O)[C@@H]2CC[C@@H]3CCN(CC(F)F)C[C@H](NC(=O)c4cc5cc(C(F)(F)P(=O)(O)O)ccc5s4)C(=O)N32)C[C@H]1c1ccccc1] | 964 [M + H]⁺ | ¹H NMR (400 MHz, DMSO-d₆) δ 8.70- 8.51 (m, 1H), 8.32- 8.28 (m, 1H), 8.18- 8.04 (m, 2H), 7.60- 7.58 (m, 1H), 7.50- 7.31 (m, 5H), 6.30- 5.92 (m, 1H), 4.95- 4.83 (m, 1H), 4.64- 4.53 (m, 1H), 4.39- 4.27 (m, 1H), 4.24- 3.91 (m, 3H), 3.85- 3.79 (m, 1H), 3.70- 3.65 (m, 1H), 3.20- 3.00 (m, 7H), 2.37- 2.28 (m, 1H), 2.13- 1.98 (m, 1H), 1.92- 1.70 (m, 4H). |
| | methyl) | or | | |
| | phosphonic acid | 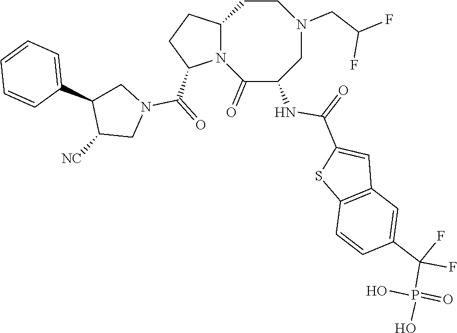 [SMILES: N#C[C@H]1CN(C(=O)[C@@H]2CC[C@@H]3CCN(CC(F)F)C[C@H](NC(=O)c4cc5cc(C(F)(F)P(=O)(O)O)ccc5s4)C(=O)N32)C[C@@H]1c1ccccc1] | | |
| | | SFC peak 2 was used for | | |
| | | biological testing | | |
| 250 | ((2-(((5S, 8S,10aR)- 8-((rel- trans)-3- cyano-4- phenyl- pyrrolidine- 1- carbonyl)-3- (methoxy- carbonyl)- 6-oxo- decahydro- pyrrolo[1,2- a][1,5] diazocin-5- yl) carbamoyl) benzo[b] thiophen-5- yl)difluoro- methyl) phosphonic acid | 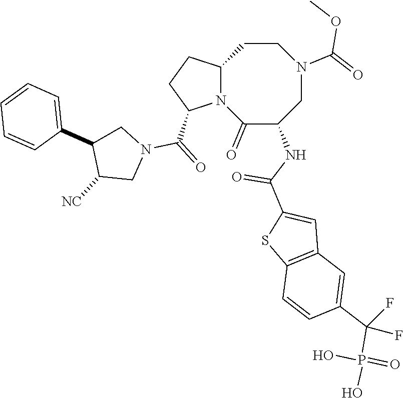 [SMILES: COC(=O)N1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(O)O)ccc3s2)C1] | 730 [M + H]⁺ | ¹H NMR (400 MHz, DMSO-d₆) δ 9.10- 8.77 (m, 1H), 8.38- 8.27 (m, 1H), 8.20- 8.03 (m, 2H), 7.60 (d, J = 8.3 Hz, 1H), 7.48- 7.29 (m, 5H), 5.01- 4.84 (m, 1H), 4.68- 4.50 (m, 1H), 4.42- 4.04 (m, 3H), 3.98- 3.61 (m, 9H), 3.37- 3.06 (m, 2H), 2.35- 2.20 (m, 1H), 2.13- 2.00 (m, 1H), 1.93- 1.70 (m, 4H). |
| 251 | ((2-(((5S, 8S,10aR)- 8-([1,1′- biphenyl]-4- ylcarba- moyl)- 3-(benzyl- carbamoyl)- 6-oxo- decahydro- pyrrolo[1,2- a][1,5] diazocin-5- yl) carbamoyl) benzo[b] thiophen-5- yl)difluoro- methyl) phosphonic acid | 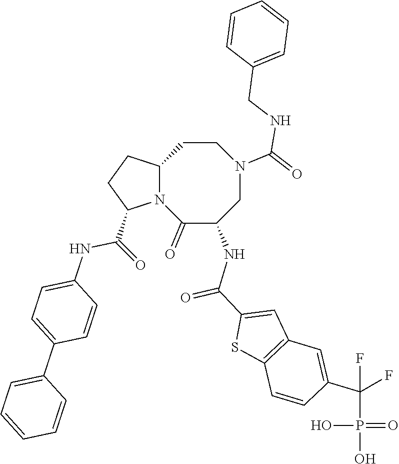 [SMILES: O=C(N[C@H]1CN(C(=O)NCc2ccccc2)CC[C@H]2CC[C@@H](C(=O)Nc3ccc(-c4ccccc4)cc3)N2C1=O)c1cc2cc(C(F)(F)P(=O)(O)O)ccc2s1] | 802 [M + H]⁺ | 1H NMR (400 MHz, DMSO-d₆) δ 10.27 (s, 1H), 8.83-8.75 (m, 1H), 8.31 (s, 1H), 8.18- 8.06 (m, 2H), 7.74- 7.57 (m, 7H), 7.49- 7.40 (m, 2H), 7.35- 7.28 (m, 5H), 7.27- 7.18 (m, 1H), 7.18- 7.10 (m, 1H), 4.90- 4.78 (m, 1H), 4.62- 4.51 (m, 1H), 4.45- 4.34 (m, 1H), 4.34- 4.25 (m, 1H), 4.24- 4.07 (m, 2H), 3.93- 3.83 (m, 1H), 3.37- 3.31 (m, 1H), 3.21- 3.13 (m, 1H), 2.38- 2.26 (m, 1H), 2.18- 1.93 (m, 3H), 1.82- 1.63 (m, 2H). |
| 252 | ((2-(((5S, 8S,10aR)- 8-([1,1′- biphenyl]-4- ylcarba- moyl)-3- ((benzyloxy) carbonyl)- 6-oxo- decahydro- pyrrolo[1,2- a][1,5] diazocin-5- yl) carbamoyl) benzo[b] thiophen-5- yl)difluoro- methyl) phosphonic acid | 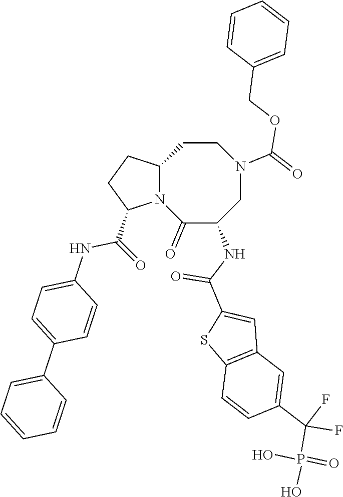 [SMILES: O=C(N[C@H]1CN(C(=O)OCc2ccccc2)CC[C@H]2CC[C@@H](C(=O)Nc3ccc(-c4ccccc4)cc3)N2C1=O)c1cc2cc(C(F)(F)P(=O)(O)O)ccc2s1] | 803 [M + H]⁺ | ¹H NMR (400 MHz, DMSO-d₆) δ 10.26- 10.16 (m, 1H), 9.06- 8.96 (m, 1H), 8.36- 8.25 (m, 1H), 8.18- 8.04 (m, 2H), 7.72- 7.57 (m, 7H), 7.47- 7.25 (m, 8H), 5.20- 4.92 (m, 3H), 4.58- 4.46 (m, 1H), 4.40- 4.27 (m, 1H), 3.88- 3.78 (m, 1H), 3.74- 3.68 (m, 1H), 3.61- 3.54 (m, 2H), 2.35- 2.20 (m, 1H), 2.19- 1.90 (m, 3H), 1.90- 1.72 (m, 2H). |
| 253 | (difluoro(2- (((5S,8S, 10aR)- 8-(methyl (phenyl) carbamoyl)- 6-oxo-3-(3- phenyl- propanoyl) decahydro- pyrrolo[1,2- a][1,5] diazocin-5- yl) carbamoyl)- 1H-indol-5- yl)methyl) phosphonic acid | 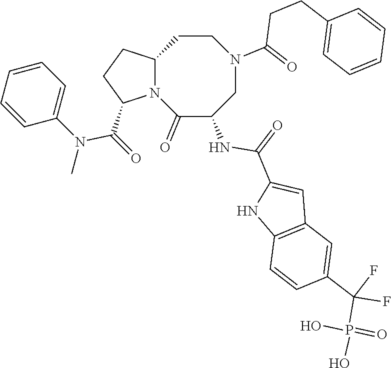 [SMILES: CN(C(=O)[C@@H]1CC[C@@H]2CCN(C(=O)CCc3ccccc3)C[C@H](NC(=O)c3cc4cc(C(F)(F)P(=O)(O)O)ccc4[nH]3)C(=O)N21)c1ccccc1] | 722.1 [M + H]⁺ | ¹H NMR (400 MHz, DMSO-d₆) δ 12.00- 11.72 (m, 1H), 8.81- 8.34 (m, 1H), 7.84 (s, 1H), 7.59-7.11 (m, 13H), 4.94-4.78 (m, 1H), 4.38-4.08 (m, 2H), 3.92-3.68 (m, 3H), 3.48-3.31 (m, 2H), 3.20 (s, 3H), 3.02-2.83 (m, 3H), 2.77-2.64 (m, 1H), 1.87-1.58 (m, 5H). |
| 254 | ((2-(((5S, 8S,10aR)- 8-([1,1′- biphenyl]-4- ylcarba- moyl)- 6-oxo-3- ((R)-2- phenyl- propanoyl) decahydro- pyrrolo[1,2- a][1,5] diazocin-5- yl) carbamoyl)- 1H-indol-5- yl)difluoro- methyl) phosphonic acid | 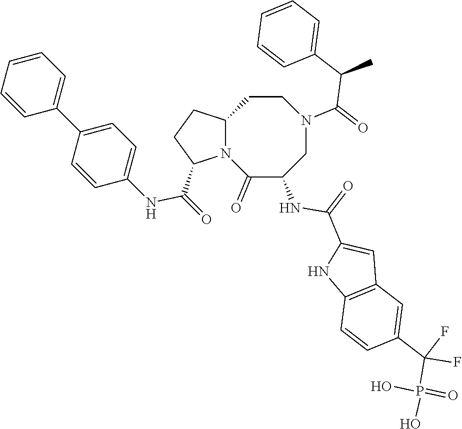 [SMILES: C[C@@H](C(=O)N1CC[C@H]2CC[C@@H](C(=O)Nc3ccc(-c4ccccc4)cc3)N2C(=O)[C@@H](NC(=O)c2cc3cc(C(F)(F)P(=O)(O)O)ccc3[nH]2)C1)c1ccccc1] | 784.3 [M + H]⁺ | ¹H NMR (400 MHz, DMSO-d₆) δ 12.14- 11.71 (m, 1H), 10.68- 10.08 (m, 1H), 8.84- 8.49 (m, 1H), 7.86- 7.17 (m, 18H), 4.88- 4.76 (m, 1H), 4.59- 4.36 (m, 2H), 4.21- 4.06 (m, 2H), 3.75- 3.54 (m, 3H), 2.29- 1.70 (m, 6H), 1.41 (d, J = 6.7 Hz, 3H). |
| 255 | ((2-(((5S, 8S,10aR)- 8-([1,1′- biphenyl]-4- ylcarba- moyl)- 6-oxo-3- ((S)-2- phenyl- propanoyl) decahydro- pyrrolo[1,2- a][1,5] diazocin-5- yl) carbamoyl)- 1H-indol-5- yl)difluoro- methyl) phosphonic acid | 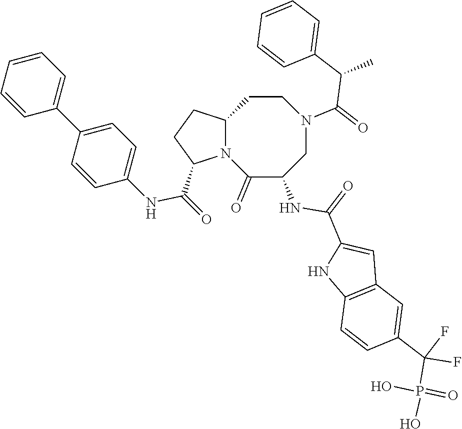 [SMILES: C[C@H](C(=O)N1CC[C@H]2CC[C@@H](C(=O)Nc3ccc(-c4ccccc4)cc3)N2C(=O)[C@@H](NC(=O)c2cc3cc(C(F)(F)P(=O)(O)O)ccc3[nH]2)C1)c1ccccc1] | 784.3 [M + H]⁺ | ¹H NMR (400 MHz, DMSO-d₆) δ 12.41- 11.72 (m, 1H), 10.42- 10.17 (m, 1H), 8.86- 8.17 (m, 1H), 7.99- 7.14 (m, 18H), 5.14- 4.98 (m, 1H), 4.78- 4.66 (m, 1H), 4.64- 4.48 (m, 1H), 4.38- 4.17 (m, 1H), 4.16- 3.95 (m, 2H), 3.18- 3.05 (m, 1H), 3.02- 2.88 (m, 1H), 2.40- 2.30 (m, 1H), 2.29- 2.17 (m, 1H), 2.16- 1.91 (m, 2H), 1.86- 1.57 (m, 2H), 1.50- 1.36 (d, J = 8.0 Hz, 3H). |
| 256 | (difluoro(2- (((5S,8S, 10aR)-8-(3- (morpholine- 4- carbonyl) azetidine-1- carbonyl)- 6-oxo- decahydro- pyrrolo[1,2- a][1,5] diazocin-5- yl) carbamoyl) benzo[b] thiophen-5- yl)methyl) phosphonic acid | 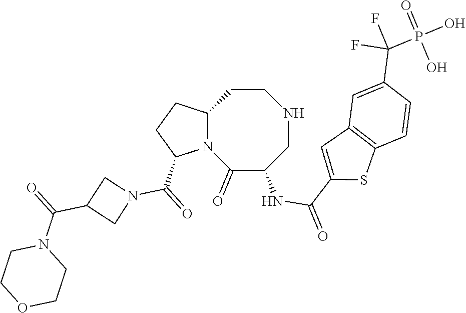 [SMILES: O=C(N[C@H]1CNCC[C@H]2CC[C@@H](C(=O)N3CC(C(=O)N4CCOCC4)C3)N2C1=O)c1cc2cc(C(F)(F)P(=O)(O)O)ccc2s1] | 668.2 [M − H]⁻ | ¹H NMR (600 MHz, DMSO-d₆) δ 8.83 (s, 1H), 8.74 (s, 1H), 8.02- 7.93 (m, 1H), 7.60 (d, J = 20.0 Hz, 1H), 7.43- 7.27 (m, 2H), 5.08 (d, J = 8.0 Hz, 1H), 4.49 (s, 2H), 4.32 (d, J = 30.7 Hz, 2H), 4.03 (s, 2H), 3.81-3.71 (m, 1H), 3.65-3.46 (m, 4H), 3.45 (s, 4H), 3.01 (s, 1H), 2.84 (s, 1H), 2.04 (s, 2H), 1.76 (d, J = 24.3 Hz, 4H), 1.22 (s, 1H), 1.14 (dt, J = 11.8, 7.3 Hz, 1H). |
| 257 | (difluoro(2- (((5S,8S, 10aR)-3- isobutyl-8- (3- (morpholine- 4- carbonyl) azetidine-1- carbonyl)- 6-oxo- decahydro- pyrrolo[1,2- a][1,5] diazocin-5- yl) carbamoyl) benzo[b] thiophen-5- yl)methyl) phosphonic acid | 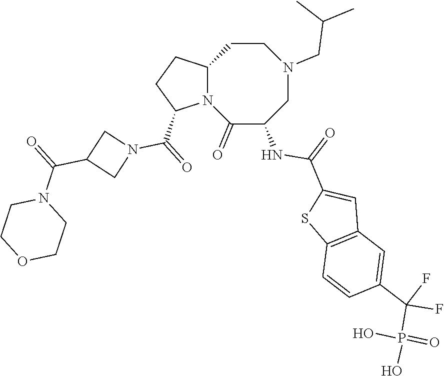 [SMILES: CC(C)CN1CC[C@H]2CC[C@@H](C(=O)N3CC(C(=O)N4CCOCC4)C3)N2C(=O)[C@@H](NC(=O)c2cc3cc(C(F)(F)P(=O)(O)O)ccc3s2)C1] | 724.0 [M − H]⁻ | ¹H NMR (400 MHz, DMSO-d₆) δ 8.90 (s, 1H), 8.25 (s, 1H), 8.04 (s, 2H), 7.58 (d, J = 8.7 Hz, 1H), 5.11 (s, 1H), 4.54 (m, 1H), 4.52- 4.41 (m, 2H), 4.33 (m, 2H), 4.10-4.04 (m, 1H), 3.99 (s, 2H), 3.91 (m, 2H), 3.78-3.70 (m, 2H), 3.45 (m, 4H), 3.33-3.15 (m, 4H), 2.26 (s, 1H), 2.05 (m, 1H), 1.79 (m, 5H), 0.92 (m, 6H). |
| 258 | (difluoro(2- (((5S,8S, 10aR)-3- ((S)-2- methyl- 3-phenyl- propanoyl)- 6-oxo-8- (pyrrolidine- 1-carbonyl) decahydro- pyrrolo[1,2- a][1,5] diazocin-5- yl) carbamoyl) benzo[b] thiophen-5- yl)methyl) phosphonic acid | 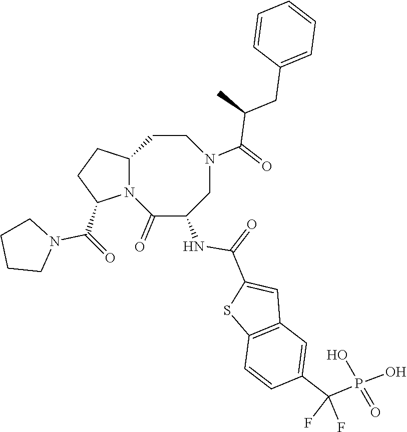 | 715.0 [M − H]⁻ | ¹H NMR (400 MHz, DMSO-d₆) δ 8.50 (m, 1H), 8.30 (m, 1H), 8.14 (m, 1H), 8.09 (s, 1H), 7.60 (m, 1H), 7.36 (m, 2H), 7.26 m, 2H), 7.18 (m, 1H), 4.48 (m, 1H), 4.35 (s, 1H), 3.89 (m, 2H), 3.81 (m, 2H), 3.59 (m, 2H), 3.47 (s, 1H), 3.40 (m, 2H), 3.21 (m, 1H), 3.15-3.01 (m, 2H), 2.87 (m, 1H), 2.74 (m, 1H), 1.92-1.83 (m, 2H), 1.80-1.33 (m, 6H), 1.14 (m, 3H). |
| 259 | (difluoro(2- (((5S,8S, 10aR)-3- ((R)- 2-methyl- 3-phenyl- propanoyl)- 6-oxo-8- (pyrrolidine- 1-carbonyl) decahydro- pyrrolo[1,2- a][1,5] diazocin-5- yl) carbamoyl) benzo[b] thiophen-5- yl)methyl) phosphonic acid | 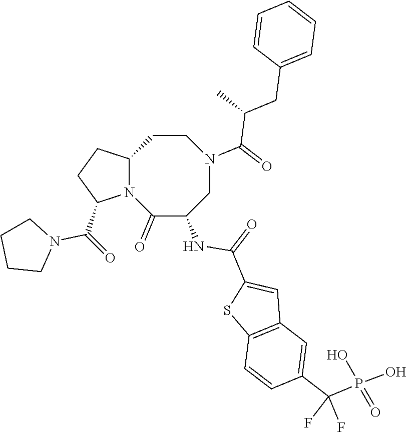 | 715.0 [M − H]⁻ | ¹H NMR (400 MHz, DMSO-d₆) δ 8.78 (m, 1H), 8.29 (m, 1H), 8.19-8.12 (m, 1H), 8.09 (s, 1H), 7.65-7.55 (m, 1H), 7.30 (m, 1H), 7.19 (m, 4H), 4.96 (s, 1H), 4.58 (m, 1H), 4.44 (m, 2H), 4.37 (m, 1H), 4.19 (s, 1H), 3.83-3.56 (m, 3H), 3.11-2.91 (m, 2H), 2.84 m, 1H), 2.31- 2.17 (m, 1H), 2.05 (m, 1H), 1.86 (m, 3H), 1.71 (m, 3H), 1.59 (m, 2H), 1.27-1.09 (m, 3H), 1.04 (m, 2H). |
| 260 | (difluoro(2- (((5S,8S, 10aR)-6- oxo-3-((R)- 3-phenyl- butanoyl)-8- (pyrrolidine- 1- carbonyl) decahydro- pyrrolo[1,2- a][1,5] diazocin-5- yl) carbamoyl) benzo[b] thiophen-5- yl)methyl) phosphonic acid | 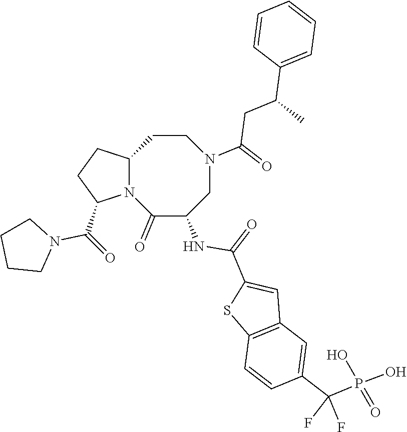 | 717.2 [M + H]⁺ | ¹H NMR (600 MHz, DMSO-d₆) δ 8.62 (m, 1H), 8.29 (m, 1H), 8.13 (m, 1H), 8.07 (s, 1H), 7.58 (m, 1H), 7.33 (m, 1H), 7.30-7.18 (m, 3H), 7.16 (m, 1H), 4.96 (m, 1H), 4.53 (m, 1H), 4.26-4.11 (m, 1H), 3.86-3.67 (m, 2H), 3.62 (m, 2H), 3.47- 3.41 (m, 1H), 3.35 (m, 2H), 3.21 (m, 1H), 3.08 (m, 1H), 2.88 (m, 1H), 2.67-2.61 (m, 1H), 2.31-2.18 (m, 1H), 2.06-1.94 (m, 1H), 1.87 (m, 3H), 1.80- 1.68 (m, 4H), 1.60- 1.44 (m, 1H), 1.26 (m, 3H). |
| 261 | (difluoro(2- (((5S,8S, 10aR)-6- oxo-3-((S)- 3-phenyl- butanoyl)-8- (pyrrolidine- 1- carbonyl) decahydro- pyrrolo[1,2- a][1,5] diazocin-5- yl) carbamoyl) benzo[b] thiophen-5- yl)methyl) phosphonic acid | 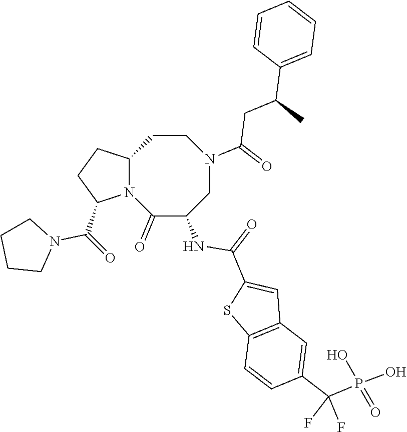 | 717.0 [M + H]⁺ | ¹H NMR (600 MHz, DMSO-d₆) δ 8.30 (m, 1H), 8.17-8.12 (m, 1H), 8.08 (m, 1H), 7.58 (m, 1H), 7.38 (m, 2H), 7.31-7.23 (m, 3H), 7.16 (m, 1H), 4.68 (m, 1H), 4.49 (m, 1H), 4.21-4.05 (m, 1H), 3.92 (m, 1H), 3.73 (m, 1H), 3.60 (m, 2H), 3.40 (m, 2H), 3.23-3.17 (m, 2H), 2.63-2.55 (m, 2H), 2.21 (m, 1H), 1.92-1.82 (m, 3H), 1.74 (m, 4H), 1.63-1.57 (m, 1H), 1.53-1.43 (m, 2H), 1.30-1.23 (m, 3H). |
| 262 | (difluoro(2- (((5S,8S, 10aR)- 3-(methoxy- carbonyl)-6- oxo-8- (pyrrolidine- 1-carbonyl) decahydro- pyrrolo[1,2- a][1,5] diazocin-5- yl) carbamoyl) benzo[b] thiophen-5- yl)methyl) phosphonic acid | 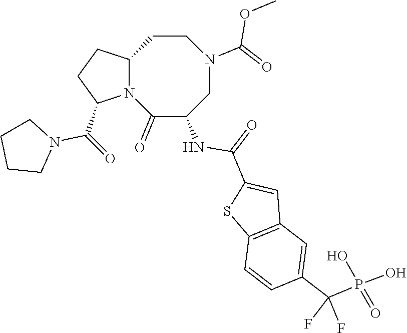 | 627.0 [M − H]⁻ | ¹H NMR (400 MHz, DMSO-d₆) δ 8.88 (m, 1H), 8.35-8.25 (m, 1H), 8.16-8.03 (m, 2H), 7.59 (m, 1H), 4.92-4.80 (m, 1H), 4.62-4.50 (m, 1H), 3.66 (s, 3H), 3.62 (s, 2H), 3.50-3.37 (m, 4H), 3.33-3.28 (m, 1H), 3.24 (m, 1H), 3.04 (m, 1H), 2.32-2.18 (m, 1H), 2.05 (m, 1H), 1.86 (m, 4H), 1.81-1.61 (m, 4H). |
| 263 | ((2-(((5S, 8S,10aR)- 3-(2,2- difluoro- ethyl)-8-(3- (morpholine- 4-carbonyl) azetidine-1- carbonyl)- 6-oxo- decahydro- pyrrolo[1,2- a][1,5] diazocin-5- yl) carbamoyl) benzo[b] thiophen-5- yl)difluoro- methyl) phosphonic acid | 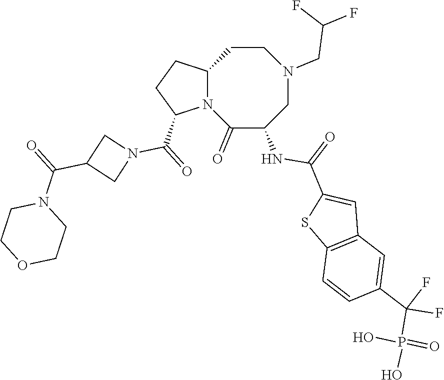 [SMILES: O=C(N[C@H]1CN(CC(F)F)CC[C@H]2CC[C@@H](C(=O)N3CC(C(=O)N4CCOCC4)C3)N2C1=O)c1cc2cc(C(F)(F)P(=O)(O)O)ccc2s1] | 732.0 [M − H]⁻ | ¹H NMR (500 MHz, DMSO-d₆) δ 8.64 (m, 1H), 8.29 (m, 1H), 8.11 (m, 1H), 8.06 (s, 1H), 7.57 (m, 1H), 6.08 (m, 1H), 4.87 (m, 1H), 4.52 (m, 1H), 4.42-4.17 (m, 3H), 4.12-3.79 (m, 3H), 3.77-3.59 (m, 2H), 3.60-3.48 (m, 4H), 3.44 (m, 2H), 3.09 (m, 4H), 2.97 (m, 2H), 2.15 (m, 1H), 2.02 (m, 1H), 1.81 (m, 1H), 1.73 (m, 1H), 1.68 (m, 2H). |
| 264 | ((2-(((5S, 8S,10aR)- 8-((2- cyano-2- methyl- propyl) (methyl) carbamoyl)- 3-(2,2-di- fluoroethyl)- 6-oxo- decahydro- pyrrolo[1,2- a][1,5] diazocin-5- yl) carbamoyl) benzo[b] thiophen-5- yl)difluoro- methyl) | 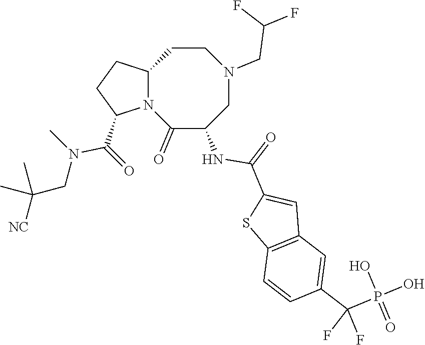 [SMILES: CN(CC(C)(C)C#N)C(=O)[C@@H]1CC[C@@H]2CCN(CC(F)F)C[C@H](NC(=O)c3cc4cc(C(F)(F)P(=O)(O)O)ccc4s3)C(=O)N21] | 674.2 [M − H]⁻ | ¹H NMR (600 MHz, DMSO-d₆) δ 8.61 (m, 1H), 8.30 (s, 1H), 8.11 (m, 1H), 8.06 (s, 1H), 7.57 (m, 1H), 6.07 (m, 1H), 4.87 (s, 1H), 4.79 (m, 1H), 4.31 (m, 1H), 3.17-3.06 (m, 7H), 3.04-2.94 (m, 4H), 2.34 (m, 1H), 2.07 (m, 1H), 1.79-1.62 (m, 4H), 1.37 (m, 1H), 1.28 (s, 3H), 1.25 (s, 3H), 1.23-1.09 (m, 1H). |
| | phosphonic | | | |
| | acid | | | |
| 265 | ((2-(((5S, 8S,10aR)- 8-([1,1′- biphenyl]-2- ylcarba- moyl)- 6-oxo-3-(2- phenyl- acetyl) decahydro- pyrrolo[1,2- a][1,5] diazocin-5- yl) carbamoyl)- 1H-indol-5- yl)difluoro- methyl) phosphonic acid | 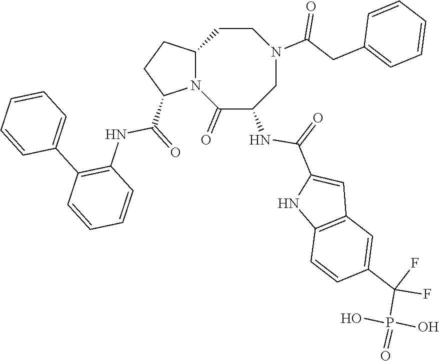 [SMILES: O=C(N[C@H]1CN(C(=O)Cc2ccccc2)CC[C@H]2CC[C@@H](C(=O)Nc3ccccc3-c3ccccc3)N2C1=O)c1cc2cc(C(F)(F)P(=O)(O)O)ccc2[nH]1] | 770.2 [M + H]⁺ | ¹H NMR (500 MHz, Methanol-d₄) δ 12.66 (m, 1H), 10.23 (s, 1H), 9.64-9.17 (m, 1H), 8.64 (m, 1H), 8.31 (m, 2H), 8.26-8.23 (m, 3H), 8.22-8.09 (m, 7H), 8.06 (m, 4H), 7.99 (m, 1H), 5.85 (m, 1H), 5.25 (m, 1H), 5.09 (s, 1H), 4.84-4.68 (m, 4H), 2.94-2.86 (m, 1H), 2.82 (m, 1H), 2.70 (s, 1H), 2.64-2.56 (m, 1H), 2.55-2.45 (m, 2H). |
| 266 | ((2-(((5S, 8S,10aR)- 8-((2′- cyano-[1,1′- biphenyl]-4- yl) carbamoyl)- 6-oxo-3-(2- phenyl- acetyl) decahydro- pyrrolo[1,2- a][1,5] diazocin-5- yl) carbamoyl)- 1H-indol-5- yl)difluoro- methyl) phosphonic acid | 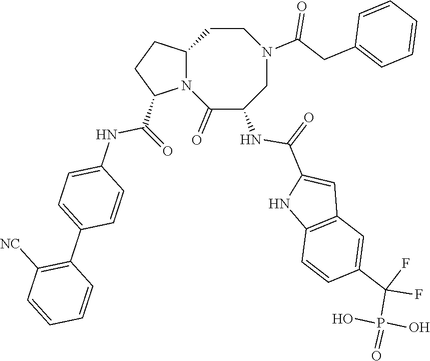 [SMILES: N#Cc1ccccc1-c1ccc(NC(=O)[C@@H]2CC[C@@H]3CCN(C(=O)Cc4ccccc4)C[C@H](NC(=O)c4cc5cc(C(F)(F)P(=O)(O)O)ccc5[nH]4)C(=O)N32)cc1] | 795.2 [M + H]⁺ | ¹H NMR (500 MHz, Methanol-d₄) δ 12.66 (m, 1H), 11.18 (m, 1H), 9.38 (m, 1H), 8.72 (m, 1H), 8.68-8.54 (m, 4H), 8.41 (m, 1H), 8.35 (m, 3H), 8.29 (m, 1H), 8.19-8.07 (m, 6H), 8.04-7.97 (m, 1H), 5.94 (m, 1H), 5.36 (m, 1H), 5.27-5.15 (m, 1H), 4.80 (m, 1H), 4.72 (m, 2H), 4.61 (m, 3H), 3.15-3.08 (m, 1H), 2.91 (m, 1H), 2.86-2.39 (m, 4H). |
| 267 | ((2-(((5S, 8S,10aR)- 8-([1,1′- biphenyl]-4- ylcarba- moyl)- 6-oxo-3-(3- (pyridin-3- yl) propanoyl) decahydro- pyrrolo[1,2- a][1,5] diazocin-5- yl) carbamoyl) benzo[b] thiophen-5- yl)difluoro- methyl) phosphonic acid | 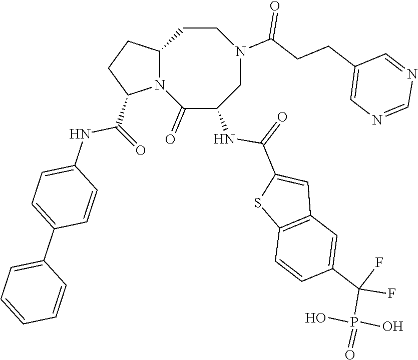 [SMILES: O=C(N[C@H]1CN(C(=O)CCc2cncnc2)CC[C@H]2CC[C@@H](C(=O)Nc3ccc(-c4ccccc4)cc3)N2C1=O)c1cc2cc(C(F)(F)P(=O)(O)O)ccc2s1] | 801.0 [M − H]⁻ | ¹H NMR (600 MHz, DMSO-d₆) δ 10.22 (m, 1H), 9.05-8.97 (m, 1H), 8.74 (s, 1H), 8.70 (s, 1H), 8.28 (m, 1H), 8.13 (m, 1H), 8.06 (m, 1H), 7.67 (m, 2H), 7.62-7.51 (m, 5H), 7.42 (m, 2H), 7.30 (m, 1H), 5.05 (s, 1H), 4.53- 4.45 (m, 1H), 4.23 (m, 1H), 3.88 (s, 1H), 3.74 (m, 2H), 3.57 (s, 2H), 2.99-2.69 (m, 4H), 2.28-2.23 (m, 1H), 2.09-2.04 (m, 1H), 1.97-1.92 (m, 1H), 1.89 (s, 1H), 1.81- 1.72 (m, 2H). |
| 268 | ((2-(((5S, 8S,10aR)- 8-([1,1′- biphenyl]-4- ylcarba- moyl)- 6-oxo-3- (phenethyl- sulfonyl) decahydro- pyrrolo[1,2- a][1,5] diazocin-5- yl) carbamoyl) benzo[b] thiophen-5- yl)difluoro- methyl) phosphonic acid | 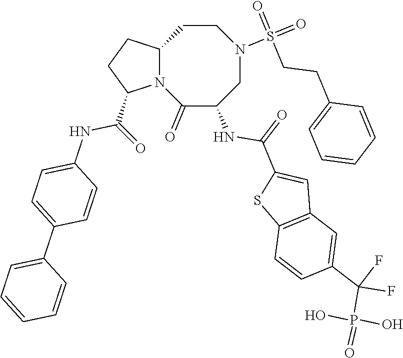 [SMILES: O=C(N[C@H]1CN(S(=O)(=O)CCc2ccccc2)CC[C@H]2CC[C@@H](C(=O)Nc3ccc(-c4ccccc4)cc3)N2C1=O)c1cc2cc(C(F)(F)P(=O)(O)O)ccc2s1] | 837.0 [M + H]⁺ | ¹H NMR (600 MHz, DMSO-d₆) δ 10.25 (s, 1H), 8.84-8.73 (m, 1H), 8.26 (s, 1H), 8.11-8.01 (m, 2H), 7.67 (m, 2H), 7.60 (m, 5H), 7.42 (m, 2H), 7.31 (m, 3H), 7.27 (m, 2H), 7.19 (m, 1H), 4.93 (m, 1H), 4.54 (m, 1H), 4.44 (m, 1H), 3.83 (m, 1H), 3.75 (m, 1H), 3.55 (m, 1H), 3.49-3.44 (m, 1H), 3.39 (s, 1H), 3.26- 3.21 (m, 1H), 3.00 (m, 2H), 2.29 m, 1H), 2.15 (m, 1H), 1.97 (m, 1H), 1.94-1.87 (m, 1H), 1.82 (m, 2H). |
| 269 | ((2-(((5S, 8S,10aR)- 3-(3- cyclohexyl- 3-methyl- butanoyl)- 6-oxo-8- (3-(pyridin- 3- yl)azetidine- 1-carbonyl) decahydro- pyrrolo[1,2- a][1,5] diazocin-5- yl) carbamoyl) benzo[b] thiophen-5- yl)difluoro- methyl) phosphonic acid | 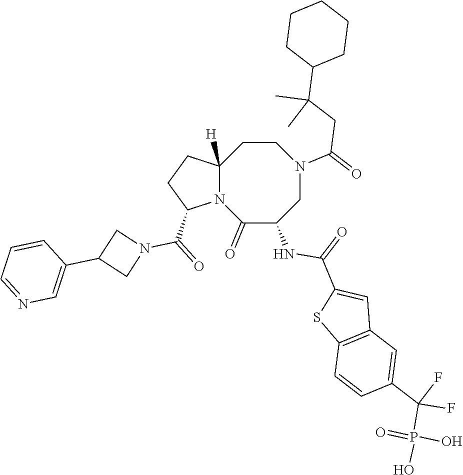 [SMILES: CC(C)(CC(=O)N1CC[C@H]2CC[C@@H](C(=O)N3CC(c4cccnc4)C3)N2C(=O)[C@@H](NC(=O)c2cc3cc(C(F)(F)P(=O)(O)O)ccc3s2)C1)C1CCCCC1] | | ¹H NMR (400 MHz, METHANOL-d₄) δ 8.81-8.47 (m, 2H), 8.47-8.24 (m, 1H), 8.24-8.16 (m, 1H), 8.13-8.05 (m, 1H), 7.97-7.87 (m, 1H), 7.82-7.65 (m, 2H), 5.08-4.98 (m, 1H), 4.76-4.65 (m, 2H), 4.52 (t, J = 8.8 Hz, 1H), 4.47-4.39 (m, 1H), 4.34-4.26 (m, 1H), 4.11-3.99 (m, 3H), 3.91-3.82 (m, 1H), 3.63-3.37 (m, 2H), 2.78-2.67 (m, 1H), 2.49-2.40 (m, 1H), 2.37-2.27 (m, 1H), 2.22-2.13 (m, 1H), 2.12-2.01 (m, 2H), 1.96-1.71 (m, 6H), 1.70-1.63 (m, 1H), 1.41-1.14 (m, 6H), 1.05 (s, 2H), 1.03-0.96 (m, 4H) |
| 270 | ((7-(((5S, 8S,10aR)- 8- (cyclopropyl (methyl) carbamoyl)- 3-(5- methyl-1H- indazole-3- carbonyl)- 6-oxo- decahydro- pyrrolo[1,2- a][1,5] diazocin-5- yl) carbamoyl) naphthalen- 2- yl)difluoro- methyl) phosphonic acid | 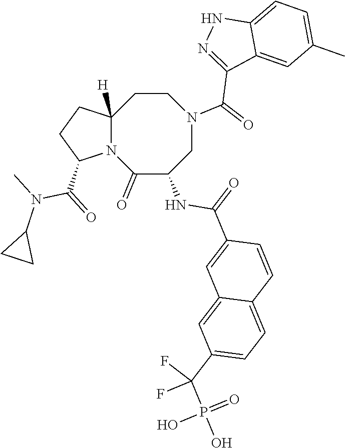 [SMILES: Cc1ccc2[nH]nc(C(=O)N3CC[C@H]4CC[C@@H](C(=O)N(C)C5CC5)N4C(=O)[C@@H](NC(=O)c4ccc5ccc(C(F)(F)P(=O)(O)O)cc5c4)C3)c2c1] | | ¹H NMR (400 MHz, METHANOL-d₄) δ ppm 8.48-8.54 (m, 1H) 8.18-8.27 (m, 1H) 8.00-8.06 (m, 1H) 7.98 (m, 1H) 7.91- 7.95 (m, 1H) 7.89 (s, 1H) 7.77-7.84 (m, 1H) 7.41 (d, J = 8.0 Hz, 1H) 7.25 (d, J = 8.0 Hz, 1H) 5.43-5.51 (m, 1H) 5.33-5.41 (m, 1H) 4.52-4.59 (m, 1H) 4.38-4.47 (m, 1H) 3.62-3.74 (m, 2H) 2.85-2.94 (m, 4H) 2.44-2.48 (m, 4H) 2.26-2.39 (m, 2H) 1.97-2.09 (m, 2H) 1.93 (m, 1H) 1.68- 1.88 (m, 1H) 1.01 (m, 1H) 0.90 (m, 2H) 0.73-0.81 (m, 1H) |
| 271 | ((7-(((5S, 8S,10aR)- 8-(dimethyl- carbamoyl)- 3-(5-methyl- 1H-indazole- 3-carbonyl)- 6-oxo- decahydro- pyrrolo[1,2- a][1,5] diazocin-5- yl) carbamoyl) naphthalen- 2- yl)difluoro- methyl) phosphonic acid | 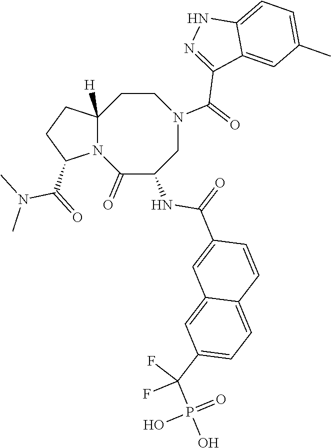 | | ¹H NMR (400 MHz, METHANOL-d₄) δ 8.51 (s, 1H) 8.25 (s, 1H) 7.92-7.99 (m, 3H) 7.83-7.90 (m, 2H) 7.41 (d, J = 12.0 Hz, 1H) 7.25 (d, J = 8.0 Hz, 1H) 5.42-5.50 (m, 1H) 4.95 (m, 1H) 4.51- 4.61 (m, 1H) 4.38- 4.47 (m, 1H) 3.64- 3.75 (m, 2H) 3.09- 3.18 (m, 4H) 2.93 (s, 3H) 2.43-2.49 (m, 4H) 2.24-2.39 (m, 2H) 1.89-2.06 (m, 3H) |
| 272 | (difluoro(2- (((5S,8S, 10aR)-3- (imidazo [1,2-a] pyridin-7- ylmethyl)-8- (methyl- carbamoyl)- 6-oxo- decahydro- pyrrolo[1,2- a][1,5] diazocin-5- yl) carbamoyl) benzo[b] thiophen-5- yl)methyl) phosphonic acid | 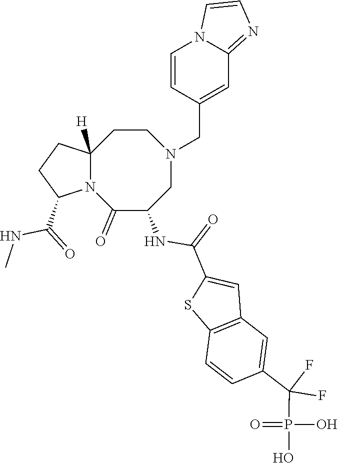 [SMILES: CNC(=O)[C@@H]1CC[C@@H]2CCN(Cc3ccn4ccnc4c3)C[C@H](NC(=O)c3cc4cc(C(F)(F)P(=O)(O)O)ccc4s3)C(=O)N21] | 661.1 [M + H]⁺ | ¹H NMR (400 MHz, METHANOL-d₄) δ 8.54 (d, J = 7.2 Hz, 1H), 8.16 (s, 1H), 8.03- 7.98 (m, 2H), 7.89 (d, J = 8.8 Hz, 1H), 7.77- 7.70 (m, 3H), 7.39 (d, J = 7.2 Hz, 1H), 5.24- 5.14 (m, 1H), 4.57- 4.46 (m, 2H), 4.11- 3.92 (m, 2H), 3.16- 3.10 (m, 1H), 3.06 (m, 2H), 2.82 (s, 3H), 2.32-2.20 (m, 2H), 2.13-1.97 (m, 2H), 1.89-1.83 (m, 1H), 1.75-1.66 (m, 1H), 1.43-1.43 (m, 1H) |
| 273 | (difluoro(2- (((5S,8S, 10aR)-3- (imidazo [1,2-a] pyridin-7- ylmethyl)-6- oxo-8-(3- (pyridin-3- yl)azetidine- 1- carbonyl) decahydro- pyrrolo[1,2- a][1,5] diazocin-5- yl) carbamoyl) benzo[b] thiophen-5- yl)methyl) phosphonic acid | 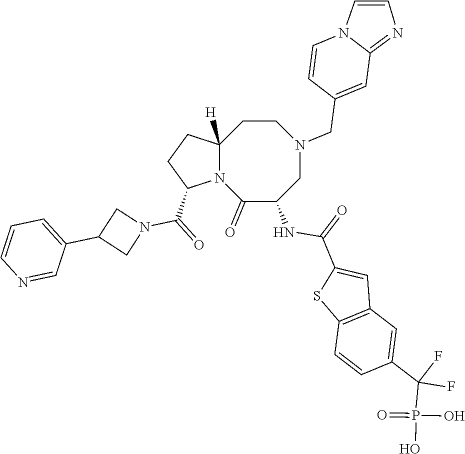 [SMILES: O=C(N[C@H]1CN(Cc2ccn3ccnc3c2)CC[C@H]2CC[C@@H](C(=O)N3CC(c4cccnc4)C3)N2C1=O)c1cc2cc(C(F)(F)P(=O)(O)O)ccc2s1] | 764.2 [M + H]⁺ | ¹H NMR (400 MHz, METHANOL-d₄) δ 8.59 (m, 1H), 8.55- 8.40 (m, 2H), 8.14 (d, J = 5.6 Hz, 1H), 8.07- 7.98 (m, 2H), 7.98- 7.93 (m, 1H), 7.91- 7.85 (m, 1H), 7.79- 7.69 (m, 3H), 7.55 (d, J = 7.2 Hz, 1H), 7.51- 7.36 (m, 1H), 5.24- 4.97 (m, 2H), 4.75- 4.70 (m, 1H), 4.57- 4.31 (m, 3H), 4.19- 3.94 (m, 4H), 3.06 (m, 4H), 2.35-2.22 (m, 2H), 2.15-1.97 (m, 2H), 1.89 (m, 1H), 1.71-1.61 (m, 1H) |
| 274 | (difluoro(2- (((5S,8S, 10aR)-3-(2- (4-methyl- piperazin-1- yl)benzyl)-6- oxo-8-(3- (pyridin-3- yl)azetidine- 1- carbonyl) decahydro- pyrrolo[1,2- a][1,5] diazocin-5- yl) carbamoyl) benzo[b] thiophen-5- yl)methyl) phosphonic acid | 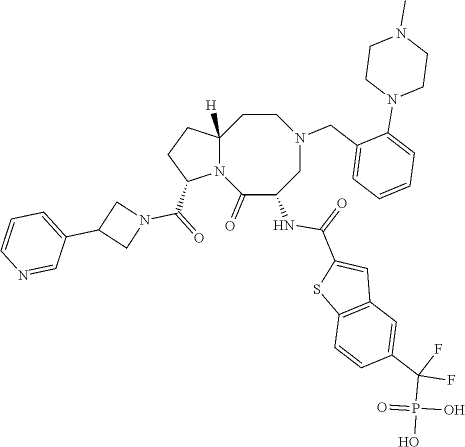 [SMILES: CN1CCN(c2ccccc2CN2CC[C@H]3CC[C@@H](C(=O)N4CC(c5cccnc5)C4)N3C(=O)[C@@H](NC(=O)c3cc4cc(C(F)(F)P(=O)(O)O)ccc4s3)C2)CC1] | 822.6 [M + H]⁺ | ¹H NMR (400 MHz, DMSO-d₆) δ 8.82- 8.67 (m, 1H), 8.61- 8.42 (m, 2H), 8.33- 8.20 (m, 1H), 8.13- 7.94 (m, 2H), 7.86- 7.80 (m, 1H), 7.70- 7.48 (m, 2H), 7.45- 7.31 (m, 1H), 7.24- 7.10 (m, 1H), 7.08- 6.86 (m, 2H), 5.02 (d, J = 7.2 Hz, 1H), 4.94- 4.58 (m, 1H), 4.54- 4.21 (m, 4H), 3.96- 3.79 (m, 4H), 3.43 (s, 2H), 2.99-2.78 (m, 11H), 2.21-2.07 (m, 2H), 1.95-1.84 (m, 1H), 1.82-1.68 (m, 2H), 1.65-1.56 (m, 1H) |
| 275 | (difluoro(2- (((5S,8S, 10aR)-3-(3- methoxy-4- (4-methyl- 1H-imidazol- 1-yl)benzyl)- 6-oxo-8-(3- (pyridin-3- yl)azetidine- 1- carbonyl) decahydro- pyrrolo[1,2- a][1,5] diazocin-5- yl) carbamoyl) benzo[b] thiophen-5- yl)methyl) phosphonic acid | 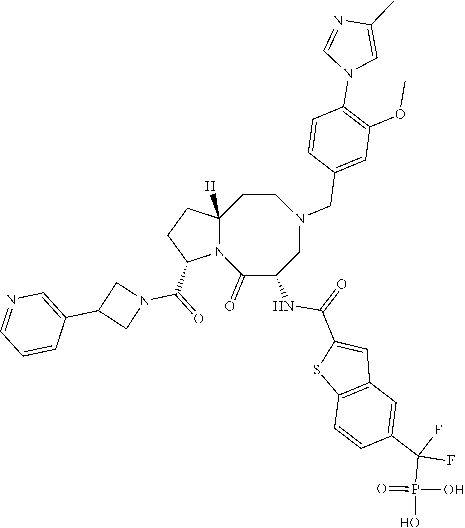 [SMILES: COc1cc(CN2CC[C@H]3CC[C@@H](C(=O)N4CC(c5cccnc5)C4)N3C(=O)[C@@H](NC(=O)c3cc4cc(C(F)(F)P(=O)(O)O)ccc4s3)C2)ccc1-n1cnc(C)c1] | 834.2 [M + H]⁺ | ¹H NMR (400 MHz, METHANOL-d₄) δ 8.62-8.53 (m, 1H), 8.48-8.42 (m, 1H), 8.30-8.22 (m, 1H), 8.16 (s, 1H), 8.09-8.00 (m, 2H), 7.95-7.88 (m, 1H), 7.73 (d, J = 8.4 Hz, 1H), 7.50-7.38 (m, 2H), 7.36-7.28 (m, 1H), 7.25-7.16 (m, 2H), 5.24-5.10 (m, 1H), 4.72-4.66 (m, 1H), 4.54 (s, 2H), 4.47-4.40 (m, 1H), 4.09-3.86 (m, 8H), 3.14-2.96 (m, 4H), 2.33-2.24 (m, 5H), 2.11-1.98 (m, 2H), 1.94-1.84 (m, 1H), 1.77-1.65 (m, 1H) |
| 276 | ((2-(((5S, 8S,10aR)- 8-((2′- cyano-3- fluoro-[1,1′- biphenyl]-4- yl) carbamoyl)- 6-oxo-3-(2- phenyl- acetyl) decahydro- pyrrolo[1,2- a][1,5] diazocin-5- yl) carbamoyl)- 1H-indol-5- yl)difluoro- methyl) phosphonic acid | 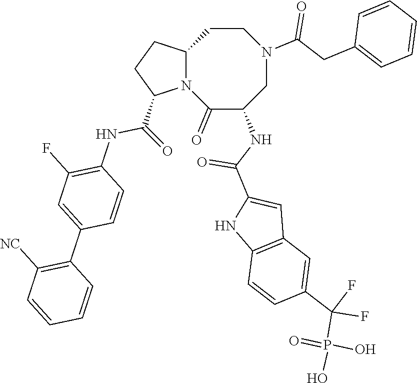 [SMILES: N#Cc1ccccc1-c1ccc(NC(=O)[C@@H]2CC[C@@H]3CCN(C(=O)Cc4ccccc4)C[C@H](NC(=O)c4cc5cc(C(F)(F)P(=O)(O)O)ccc5[nH]4)C(=O)N32)c(F)c1] | 814.0 [M + H]⁺ | |
| 277 | (difluoro(2- (((5S,8S, 10aR)-8-(3- (morpholine- 4- carbonyl) azetidine-1- carbonyl)-6- oxo-3-(2,2,2- trifluoro- ethyl) decahydro- pyrrolo[1,2- a][1,5] diazocin-5- yl) carbamoyl) benzo[b] thiophen-5- yl)methyl) phosphonic acid | 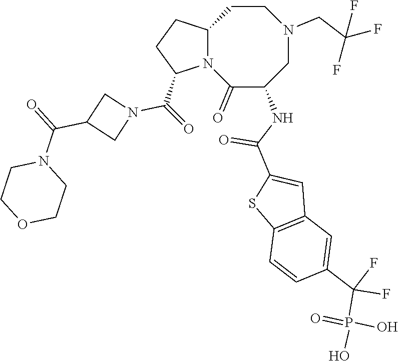 [SMILES: O=C(N[C@H]1CN(CC(F)(F)F)CC[C@H]2CC[C@@H](C(=O)N3CC(C(=O)N4CCOCC4)C3)N2C1=O)c1cc2cc(C(F)(F)P(=O)(O)O)ccc2s1] | 752.0 [M + H]⁺ | ¹H NMR (600 MHz, DMSO-d₆) δ 8.63 (m, 1H), 8.28 (m, 1H), 8.11 (m, 1H), 8.06 (s, 1H), 7.57 (m, 1H), 4.87 (m, 1H), 4.51 (m, 1H), 4.37-4.13 (m, 3H), 4.07-3.86 (m, 2H), 3.75-3.55 (m, 2H), 3.53 (m, 4H), 3.47-3.42 (m, 2H), 3.25 (m, 2H), 3.23-3.17 (m, 1H), 3.10-2.71 (m, 4H), 2.16 (m, 1H), 2.06-1.99 (m, 1H), 1.85-1.60 (m, 4H). |
| 278 | ((2-(((5S, 8S,10aR)- 3-ethyl-8- (3- (morpholine- 4-carbonyl) azetidine-1- carbonyl)-6- oxo- decahydro- pyrrolo[1,2- a][1,5] diazocin-5- yl) carbamoyl) benzo[b] thiophen-5- yl)difluoro- methyl) phosphonic acid | 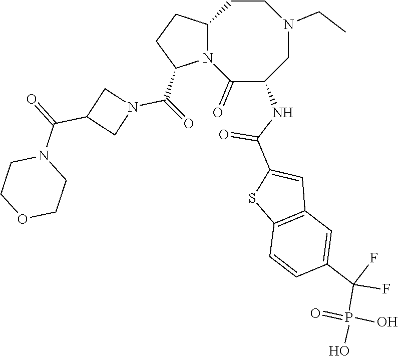 [SMILES: CCN1CC[C@H]2CC[C@@H](C(=O)N3CC(C(=O)N4CCOCC4)C3)N2C(=O)[C@@H](NC(=O)c2cc3cc(C(F)(F)P(=O)(O)O)ccc3s2)C1] | 698.2 [M + H]⁺ | ¹H NMR (400 MHz, DMSO-d₆) δ 9.77 (s, 1H), 8.20 (s, 1H), 7.97 (s, 1H), 7.81 (s, 1H), 7.64 (s, 1H), 5.29 (s, 1H), 4.53 (m, 3H), 4.36 (m, 2H), 4.06 (m, 3H), 3.77 (s, 2H), 3.53 (m, 8H), 3.27 (s, 4H), 2.06 (m, 1H), 1.82 (s, 4H), 1.29 (s, 3H). |
| 279 | ((2-(((5S, 8S,10aR)- 8-((rel- trans)-3- cyano-4- phenyl- pyrrolidine- 1- carbonyl)-3- isobutyl-6- oxo- decahydro- pyrrolo[1,2- a][1,5] diazocin-5- yl) carbamoyl) benzo[b] thiophen-5- yl)difluoro- methyl) phosphonic | 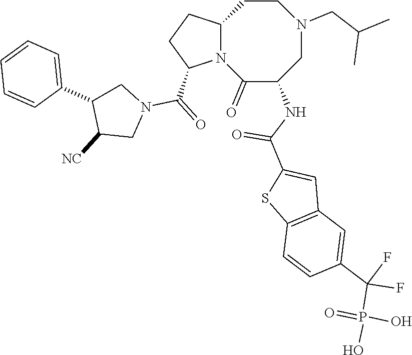 [SMILES: CC(C)CN1CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3s2)C1] | 728.2 [M + H]⁺ | ¹H NMR (600 MHz, DMSO-d₆) δ 9.20 (s, 1H), 8.24 (m, 1H), 8.13 (m, 2H), 7.60 (m, 1H), 7.51-7.36 (m, 4H), 7.33 (m, 1H), 5.51 (s, 1H), 4.89 (m, 1H), 4.68 (s, 1H), 4.37-4.05 (m, 2H), 4.04-3.77 (m, 4H), 3.67 (m, 3H), 3.51 (m, 1H), 3.27 (s, 2H), 2.08 (s, 3H), 1.85 (m, 3H), 1.09-0.90 (m, 6H). |
| | acid | | | |

### Phosphonate Esters and Amides DATA TABLES:

### General Scheme 1: Phosphonate Esters or Amides

The following compounds in Table 18 were prepared according to the representative procedure described above for the synthesis of S,S′-(((((7-(((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate) (1) utilizing the appropriate starting materials and modifications.

TABLE 18

| Com- | | | | |
|---|---|---|---|---|
| pound | Name | Structure | LCMS | NMR |
| 280 | S,S′-(((((2- (((5S,8S, 10aR)- 3-acetyl-8- (methyl (phenyl) carbamoyl)- 6- oxodecahydro- pyrrolo[1,2- a][1,5] diazocin-5- yl)carbamoyl) benzo[b] thiophen-5- yl)difluoro- methyl)phos- phoryl)bis (oxy))bis (ethane- | 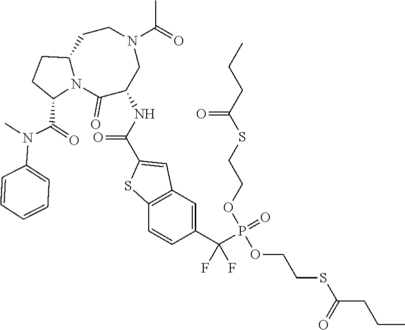 [SMILES: CCCC(=O)SCCOP(=O)(OCCSC(=O)CCC)C(F)(F)c1ccc2sc(C(=O)N[C@H]3CN(C(C)=O)CC[C@H]4CC[C@@H](C(=O)N(C)c5ccccc5)N4C3=O)cc2c1] | 909.3 [M + H]⁺ | ¹H NMR (400 MHz, METHANOL--d₄) δ 8.27-8.01 (m, 3H), 7.70-7.61 (m, 1H), 7.51-7.34 (m, 5H), 5.08-4.92 (m, 1H), 4.47-4.36 (m, 1H), 4.33-4.15 (m, 5H), 4.06-3.79 (m, 2H), 3.71-3.46 (m, 2H), 3.28-3.22 (m, 3H), 3.22-3.12 (m, 4H), 2.59-2.48 (m, 4H), 2.34-2.25 (m, 3H), 2.08-1.87 (m, 5H), 1.84-1.72 (m, 1H), 1.70-1.58 (m, 4H), 0.97-0.87 (m, 6H). |
| | 2,1-diyl))di- | | | |
| | butanethioate | | | |
| 281 | ((((2- (((5S,8S, 10aR)- 8-((3S,4R or 3R,4S)-3- cyano-4- phenyl- pyrrolidine-1- carbonyl)-3- ethyl-6- oxodecahydro- pyrrolo[1,2- a][1,5] diazocin-5- yl)carbamoyl) benzo[b] thiophen-5- yl)difluoro- methyl)phos- phoryl) | 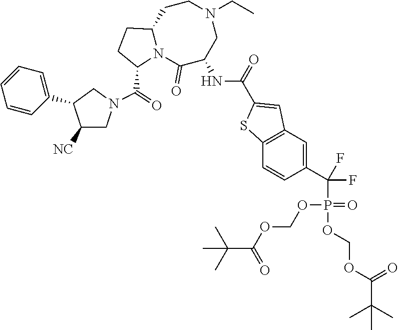 [SMILES: CCN1CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(OCOC(=O)C(C)(C)C)OCOC(=O)C(C)(C)C)ccc3s2)C1] | 928.4 [M + H]⁺ | ¹H NMR (400 MHz, ACETONITRILE-d₃) δ 8.11 (d, J = 6.8 Hz, 1H), 8.08-8.02 (m, 1H), 7.98 (d, J = 5.1 Hz, 1H), 7.82 (d, J = 6.8 Hz, 1H), 7.75 (d, J = 7.1 Hz, 1H), 7.63- 7.56 (m, 1H), 7.46- 7.30 (m, 5H), 5.70- 5.58 (m, 4H), 4.94- 4.85 (m, 1H), 4.65- 4.56 (m, 1H), 4.45- 4.30 (m, 1.4H), 4.29- 4.21 (m, 0.6H), 4.07 (dd, J = 11.7, 8.1 Hz, 0.6H), 4.00 (dd, J = 11.6, 7.7 Hz, 0.4H), 3.79 (t, J = 9.5 Hz, 0.4H), 3.73-3.33 (m, |
| | bis(oxy)) | or | | 3.6H), 3.20-3.12 (m, |
| | bis(methylene) bis(2,2- dimethyl- propanoate) | 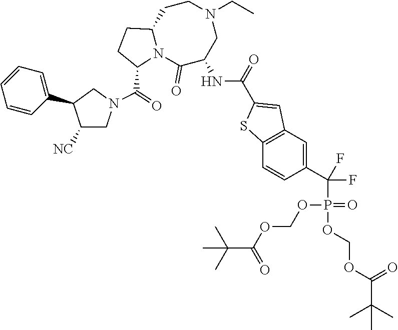 [SMILES: CCN1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(OCOC(=O)C(C)(C)C)OCOC(=O)C(C)(C)C)ccc3s2)C1] | | 1H), 3.03-2.84 (m, 2H), 2.82-2.69 (m, 3H), 2.42-2.25 (m, 1H), 2.13-2.01 (m, 1H), 1.92-1.58 (m, 4H), 1.16 (s, 18H), 1.12-1.06 (m, 3H). |
| | | SFC peak 2 was used for | | |
| | | biological testing | | |
| 282 | ((((2- (((5S,8S, 10aR)- 3-acetyl- 8-((3S,4R or 3R,4S)-3- cyano-4- phenyl- pyrrolidine-1- carbonyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) | 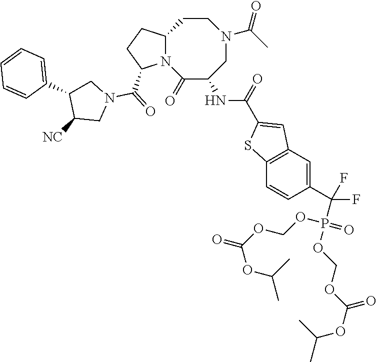 [SMILES: CC(=O)N1CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(OCOC(=O)OC(C)C)OCOC(=O)OC(C)C)ccc3s2)C1] | 946.3 [M + H]⁺ | ¹H NMR (400 MHz, CD₃CN) δ 8.16-8.08 (m, 1H), 8.07-7.87 (m, 2H), 7.63-7.55 (m, 1H), 7.45-7.29 (m, 5H), 5.69-5.57 (m, 4H), 5.01-4.78 (m, 3H), 4.72-4.62 (m, 1H), 4.37-4.15 (m, 2H), 4.13-3.85 (m, 3H), 3.80-3.68 (m, 1H), 3.67-3.56 (m, 1H), 3.55-3.29 (m, 3H), 3.25-3.16 (m, 1H), 2.47-2.30 (m, 1H), 2.28 (s, 2H), 2.14-1.97 (m, 3H), 1.92-1.61 (m, 3H), 1.26 (d, J = 6.1 Hz, 12H). |
| | diisopropyl | or | | |
| | bis(carbonate) | 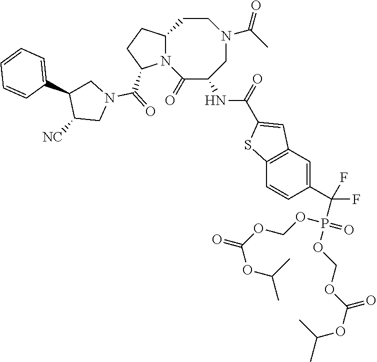 [SMILES: CC(=O)N1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(OCOC(=O)OC(C)C)OCOC(=O)OC(C)C)ccc3s2)C1] | | |
| | | SFC peak 2 was used for | | |
| | | biological testing | | |
| 283 | ((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2- | 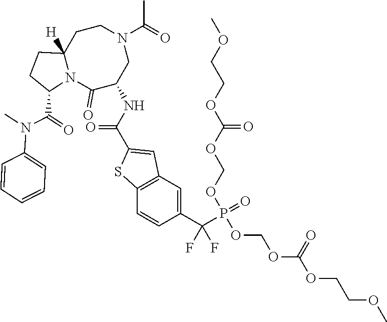 [SMILES: COCCOC(=O)OCOP(=O)(OCOC(=O)OCCOC)C(F)(F)c1ccc2sc(C(=O)N[C@H]3CN(C(C)=O)CC[C@H]4CC[C@@H](C(=O)N(C)c5ccccc5)N4C3=O)cc2c1] | 913.3 [M + H]⁺ | ¹H NMR (400 MHz, ACETONITRILE-d₃) δ 8.19-7.97 (m, 3H), 7.86-7.71 (m, 1H), 7.68-7.56 (m, 1H), 7.54-7.32 (m, 5H), 5.76-5.60 (m, 4H), 4.95-4.75 (m, 1H), 4.40-4.31 (m, 1H), 4.29-4.21 (m, 4H), 4.19-3.73 (m, 3H), 3.63-3.53 (m, 4H), 3.46-3.34 (m, 1H), 3.33-3.29 (m, 6H), 3.29-3.12 (m, 4H), 2.28 (s, 2H), 2.24-2.14 (m, 1H), 2.12-2.03 (m, 1H), 1.89-1.62 (m, 4H). |
| | methoxyethyl) | | | |
| | bis(carbonate) | | | |
| 284 | ((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) diisobutyl bis(carbonate) | 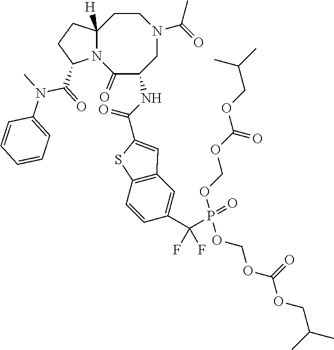 [SMILES: CC(=O)N1CC[C@H]2CC[C@@H](C(=O)N(C)c3ccccc3)N2C(=O)[C@@H](NC(=O)c2cc3cc(C(F)(F)P(=O)(OCOC(=O)OCC(C)C)OCOC(=O)OCC(C)C)ccc3s2)C1] | 909.3 [M + H]⁺ | ¹H NMR (400 MHz, ACETONITRILE-d₃) δ 8.13 (s, 1H), 8.07 (d, J = 8.8 Hz, 1H), 8.03- 7.94 (m, 2H), 7.86 (d, J = 6.4 Hz, 1H), 7.64- 7.55 (m, 1H), 7.50- 7.30 (m, 5H), 5.72- 5.61 (m, 4H), 4.93- 4.79 (m, 1H), 4.37- 4.30 (m, 1H), 4.30- 4.20 (m, 0.5H), 4.20- 4.11 (m, 1H), 4.05 (dd, J = 14.4, 2.9 Hz, 1H), 3.92 (d, J = 6.6 Hz, 5H), 3.81-3.71 (m, 0.5H), 3.40 (t, J = 10.9 Hz, 1H), 3.34-3.16 (m, 4H), 2.26 (s, 3H), 2.13-2.00 (m, 1.5H), 1.90-1.61 (m, 4.5H), |
| | | | | 0.90 (d, J = 6.6 Hz, |
| | | | | 12H). |
| 285 | ((((2- (((5S,8S, 10aR)- 3-acetyl- 8-((3R,4S or 3S,4R)-3- cyano-4- phenyl- pyrrolidine-1- carbonyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) | 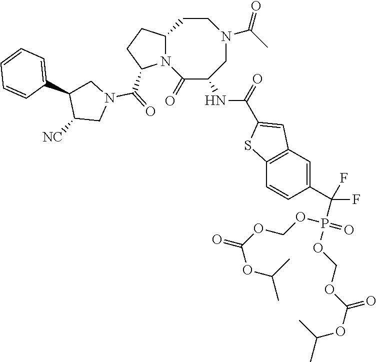 [SMILES: CC(=O)N1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(OCOC(=O)OC(C)C)OCOC(=O)OC(C)C)ccc3s2)C1] | 946.3 [M + H]⁺ | ¹H NMR (400 MHz, CD₃CN) δ 8.16-7.99 (m, 2H), 7.97-7.83 (m, 1H), 7.63-7.51 (m, 1H), 7.46-7.22 (m, 5H), 5.70-5.56 (m, 4H), 4.99-4.77 (m, 3H), 4.72-4.56 (m, 1H), 4.39-4.20 (m, 1H), 4.14-3.84 (m, 4H), 3.81-3.15 (m, 6H), 2.44-2.32 (m, 1H), 2.27 (s, 2H), 2.14-1.96 (m, 4H), 1.92-1.66 (m, 2H), 1.26 (d, J = 6.1 Hz, 12H). |
| | diisopropyl | or | | |
| | bis(carbonate) | 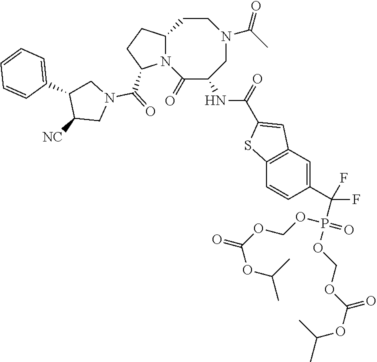 [SMILES: CC(=O)N1CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(OCOC(=O)OC(C)C)OCOC(=O)OC(C)C)ccc3s2)C1] | | |
| | | SFC peak 1 was used for | | |
| | | biological testing | | |
| 286 | ((((2-(((5S, 8S,10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl) phosphoryl) | 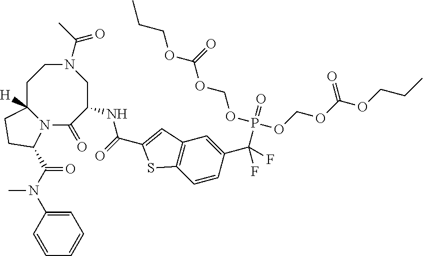 [SMILES: CCCOC(=O)OCOP(=O)(OCOC(=O)OCCC)C(F)(F)c1ccc2sc(C(=O)N[C@H]3CN(C(C)=O)CC[C@H]4CC[C@@H](C(=O)N(C)c5ccccc5)N4C3=O)cc2c1] | 881.3 [M + H]⁺ | ¹H NMR (400 MHz, CD₃CN) δ 8.14-8.09 (m, 1H), 8.07-7.98 (m, 1H), 7.81-7.79 (m, 1H), 7.65-7.57 (m, 1H), 7.51-7.32 (m, 5H), 5.72-5.59 (m, 4H), 4.90-4.78 (m, 1H), 4.37-3.68 (m, 9H), 3.42-3.30 (m, 1H), 3.26-3.10 (m, 4H), 2.27 (s, 2H), 2.12-1.98 (m, 2H), 1.91-1.72 (m, 3H), 1.69-1.60 (m, 5H), 0.95-0.86 (m, 6H). |
| | bis(oxy)) | | | |
| | bis(methylene) | | | |
| | dipropyl | | | |
| | bis(carbonate) | | | |
| 287 | ((((2-(((5S, 8S,10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl) phosphoryl) bis(oxy)) bis(methylene) bis(1- methoxy-2- methylpropan- 2-yl) bis(carbonate) | 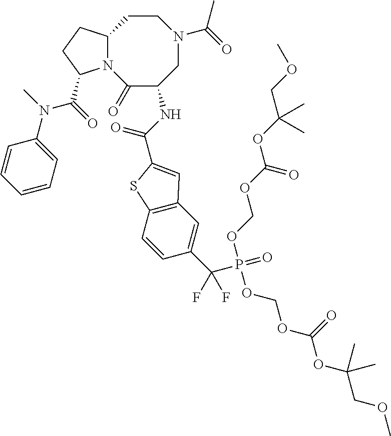 [SMILES: COCC(C)(C)OC(=O)OCOP(=O)(OCOC(=O)OC(C)(C)COC)C(F)(F)c1ccc2sc(C(=O)N[C@H]3CN(C(C)=O)CC[C@H]4CC[C@@H](C(=O)N(C)c5ccccc5)N4C3=O)cc2c1] | 969.3 [M + H]⁺ | ¹H NMR (400 MHz, CD₃CN) δ 8.16-7.97 (m, 3H), 7.85-7.81 (m, 1H), 7.65-7.55 (m, 1H), 7.50-7.45 (m, 1H), 7.43-7.40 (m, 3H), 5.67-5.56 (m, 4H), 4.91-4.78 (m, 1H), 4.36-4.12 (m, 2H), 4.09-4.02 (m, 1H), 3.96-3.72 (m, 1H), 3.48 (s, 4H), 3.44- 3.34 (m, 1H), 3.31 (s, 6H), 3.25-3.17 (m, 3H), 2.27 (s, 2H), 2.12- 1.99 (m, 1H), 1.96 (s, 2H), 1.91-1.56 (m, 5H), 1.43 (s, 12H). |
| 288 | O′1,O1- (((((2-(((5S, 8S,10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy))bis (methylene)) 2,2′- dimethyl (2S,2′S)-bis (pyrrolidine- 1,2- dicarboxylate) | 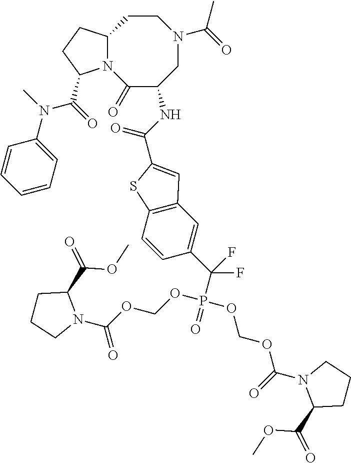 [SMILES: COC(=O)[C@@H]1CCCN1C(=O)OCOP(=O)(OCOC(=O)N1CCC[C@H]1C(=O)OC)C(F)(F)c1ccc2sc(C(=O)N[C@H]3CN(C(C)=O)CC[C@H]4CC[C@@H](C(=O)N(C)c5ccccc5)N4C3=O)cc2c1] | 1019.3 [M + H]⁺ | ¹H NMR (400 MHz, ACETONITRILE-d₃) δ 8.17-7.97 (m, 3H), 7.92-7.76 (m, 1H), 7.66-7.55 (m, 1H), 7.53-7.30 (m, 5H), 5.81-5.46 (m, 4H), 4.92-4.78 (m, 1H), 4.38-4.20 (m, 3H), 4.19-4.10 (m, 1H), 4.10-4.00 (m, 1H), 3.97-3.83 (m, 1H), 3.79-3.73 (m, 0.5H), 3.73-3.69 (m, 1.5H), 3.69-3.62 (m, 4H), 3.54-3.14 (m, 9H), 2.27 (s, 2.5H), 2.24- 2.16 (m, 1.5H), 2.12- 1.99 (m, 2H), 1.96 (s, 3H), 1.91-1.61 (m, 8H). |
| 289 | S-(2-((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- | 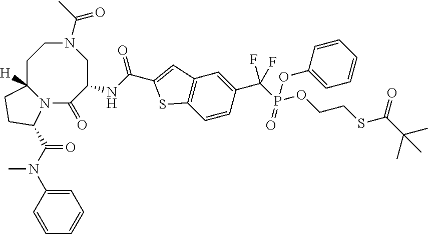 [SMILES: CC(=O)N1CC[C@H]2CC[C@@H](C(=O)N(C)c3ccccc3)N2C(=O)[C@@H](NC(=O)c2cc3cc(C(F)(F)P(=O)(OCCSC(=O)C(C)(C)C)Oc4ccccc4)ccc3s2)C1] | 869.3 [M + H]⁺ | ¹H NMR (400 MHz, METHANOL-d₄) δ 8.29-8.02 (m, 3H), 7.77-7.65 (m, 1H), 7.54-7.29 (m, 7H), 7.28-7.20 (m, 1H), 7.20-7.11 (m, 2H), 5.08-4.91 (m, 1H), 4.50-4.12 (m, 4H), 4.10-3.78 (m, 2H), 3.75-3.43 (m, 2H), 3.29-3.21 (m, 3H), 3.16-3.04 (m, 2H), 2.37-2.23 (m, 3H), 2.22-1.86 (m, 5H), |
| | methyl) | | | 1.85-1.65 (m, 1H), |
| | (phenoxy) | | | 1.16 (s, 9H). |
| | phosphoryl) | | | |
| | oxy)ethyl)2,2- | | | |
| | dimethylpro- | | | |
| | panethioate | | | |
| 290 | ((((2- (((5S,8S, 10aR)- 3-acetyl- 8-(cinnolin-6- yl(methyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- | 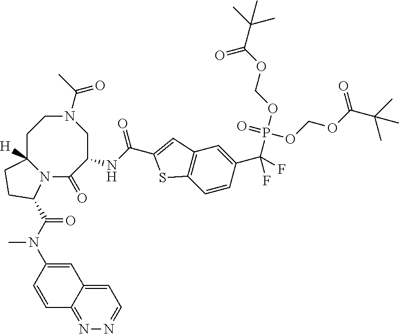 [SMILES: CC(=O)N1CC[C@H]2CC[C@@H](C(=O)N(C)c3ccc4nnccc4c3)N2C(=O)[C@@H](NC(=O)c2cc3cc(C(F)(F)P(=O)(OCOC(=O)C(C)(C)C)OCOC(=O)C(C)(C)C)ccc3s2)C1] | 929.3 [M + H]⁺ | ¹H NMR (400 MHz, ACETONITRILE-d₃) δ 9.32-9.15 (m, 1H), 8.56-8.38 (m, 1H), 8.13 (s, 1H), 8.07 (d, J = 8.6 Hz, 1H), 8.02- 7.86 (m, 4H), 7.65- 7.54 (m, 1H), 5.73- 5.61 (m, 4H), 4.97- 4.82 (m, 1H), 4.47 (br. s., 1H), 4.34-4.13 (m, 1H), 4.05 (dd, J = 14.7, 2.9 Hz, 1H), 3.95-3.81 (m, 1H), 3.80-3.66 (m, 1H), 3.50-3.16 (m, 4H), 2.31-2.15 (m, 3H), 2.12-1.97 (m, 3H), 1.92-1.62 (m, 3H), 1.19-1.13 (m, 18H). |
| | panoate) | | | |
| 291 | S,S′-(((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) | 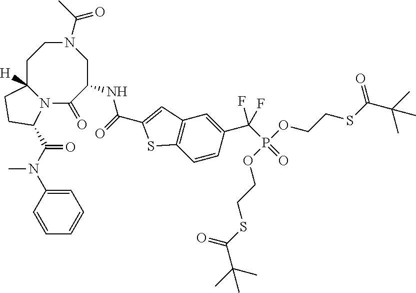 [SMILES: CC(=O)N1CC[C@H]2CC[C@@H](C(=O)N(C)c3ccccc3)N2C(=O)[C@@H](NC(=O)c2cc3cc(C(F)(F)P(=O)(OCCSC(=O)C(C)(C)C)OCCSC(=O)C(C)(C)C)ccc3s2)C1] | 937.3 [M + H]⁺ | ¹H NMR (400 MHz, METHANOL--d₄) δ 8.28-8.02 (m, 3H), 7.74-7.63 (m, 1H), 7.56-7.30 (m, 5H), 5.09-4.93 (m, 1H), 4.49-4.32 (m, 1H), 4.32-4.13 (m, 5H), 4.09-3.98 (m, 1H), 3.93-3.80 (m, 1H), 3.73-3.47 (m, 2H), 3.30-3.22 (m, 3H), 3.21-3.08 (m, 4H), 2.37-2.24 (m, 3H), 2.23-2.05 (m, 1H), 2.04-1.87 (m, 4H), 1.86-1.69 (m, 1H), 1.21 (s, 18H). |
| | bis(ethane- | | | |
| | 2,1-diyl)) | | | |
| | bis(2,2- | | | |
| | dimethylpro- | | | |
| | panethioate) | | | |
| 292 | ((((2- (((5R,8R, 10aS)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) | 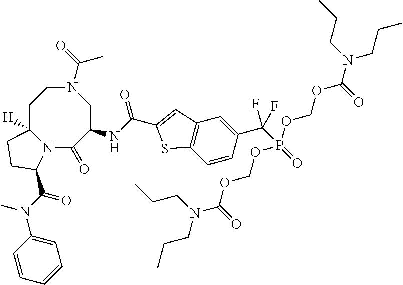 [SMILES: CCCN(CCC)C(=O)OCOP(=O)(OCOC(=O)N(CCC)CCC)C(F)(F)c1ccc2sc(C(=O)N[C@@H]3CN(C(C)=O)CC[C@@H]4CC[C@H](C(=O)N(C)c5ccccc5)N4C3=O)cc2c1] | 963.3 [M + H]⁺ | ¹H NMR (400 MHz, METHANOL-d₄) δ 8.18-8.00 (m, 3H), 7.75-7.57 (m, 1H), 7.51-7.38 (m, 4H), 7.23-7.12 (m, 1H), 5.84-5.68 (m, 4H), 5.07-4.92 (m, 1H), 4.45-4.36 (m, 1H), 4.31-4.16 (m, 1H), 4.04-4.00 (m, 1H), 3.87-3.83 (m, 1H), 3.73-3.47 (m, 2H), 3.28-3.24 (m, 3H), 3.23-3.06 (m, 8H), 2.33-2.26 (m, 3H), 2.07-1.89 (m, 5H), 1.88-1.65 (m, 1H), |
| | bis(methylene) | | | 1.63-1.45 (m, 8H), |
| | bis(dipropyl- | | | 0.93-0.78 (m, 12H). |
| | carbamate) | | | |
| 293 | ((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) | 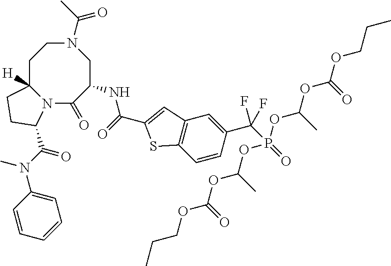 [SMILES: CCCOC(=O)OC(C)OP(=O)(OC(C)OC(=O)OCCC)C(F)(F)c1ccc2sc(C(=O)N[C@H]3CN(C(C)=O)CC[C@H]4CC[C@@H](C(=O)N(C)c5ccccc5)N4C3=O)cc2c1] | 909.3 [M + H]⁺ | ¹H NMR (400 MHz, CD₃CN) δ 8.15-7.98 (m, 3H), 7.87-7.77 (m, 1H), 7.63-7.61 (m, 1H), 7.49-7.37 (m, 4H), 6.55-6.38 (m, 2H), 4.91-4.78 (m, 1H), 4.37-3.65 (m, 9H), 3.44-3.31 (m, 1H), 3.31-3.14 (m, 4H), 2.27 (s, 2H), 2.12-1.98 (m, 2H), 1.90-1.71 (m, 3H), 1.71-1.52 (m, 8H), 1.50-1.43 (m, 3H), 0.95-0.84 (m, 6H). |
| | bis(oxy)) | | | |
| | bis(ethane- | | | |
| | 1,1-diyl) | | | |
| | dipropyl | | | |
| | bis(carbonate) | | | |
| 294 | ((((2- (((5S,8S, 10aR)- 3-acetyl- 8-(cinnolin-6- ylcarbamoyl)- 6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- | 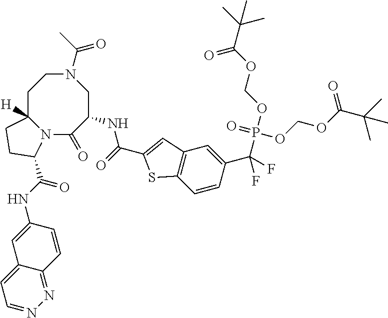 [SMILES: CC(=O)N1CC[C@H]2CC[C@@H](C(=O)Nc3ccc4nnccc4c3)N2C(=O)[C@@H](NC(=O)c2cc3cc(C(F)(F)P(=O)(OCOC(=O)C(C)(C)C)OCOC(=O)C(C)(C)C)ccc3s2)C1] | 915.3 [M + H]⁺ | ¹H NMR (400 MHz, ACETONITRILE-d₃) δ 9.41 (s, 0.3H), 9.35- 9.23 (m, 0.7H), 9.21- 9.11 (m, 1H), 8.42 (s, 0.8H), 8.40-8.27 (m, 1.2H), 8.15-7.97 (m, 3.2H), 7.96-7.76 (m, 2.8H), 7.64-7.52 (m, 1H), 5.71-5.58 (m, 4H), 5.12-4.94 (m, 1H), 4.70-4.54 (m, 1H), 4.44-4.28 (m, 1H), 4.01 (d, J = 14.9 Hz, 1H), 3.85 (d, J = 12.7 Hz, 1H), 3.75- 3.58 (m, 1H), 3.48- 3.24 (m, 2H), 2.45- 2.33 (m, 1H), 2.22 (s, 3H), 2.11-2.04 (m, |
| | dimethylpro- | | | 2H), 1.86-1.74 (m, |
| | panoate) | | | 2H), 1.17-1.13 (m, |
| | | | | 18H). |
| 295 | ((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)fluoro- methyl)phos- phoryl) bis(oxy))bis | 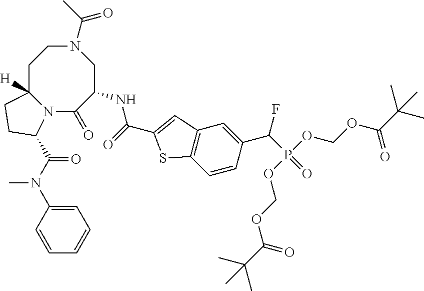 [SMILES: CC(=O)N1CC[C@H]2CC[C@@H](C(=O)N(C)c3ccccc3)N2C(=O)[C@@H](NC(=O)c2cc3cc(C(F)P(=O)(OCOC(=O)C(C)(C)C)OCOC(=O)C(C)(C)C)ccc3s2)C1] | 859.4 [M + H]⁺ | ¹H NMR (400 MHz, METHANOL-d₄) δ 8.12 (s, 1H), 8.09-7.95 (m, 2H), 7.64-7.54 (m, 1H), 7.51-7.37 (m, 5H), 6.23-6.01 (m, 1H), 5.75-5.61 (m, 4H), 5.09-4.92 (m, 1H), 4.47-4.22 (m, 2H), 4.20-3.97 (m, 1H), 3.91-3.80 (m, 1H), 3.78-3.60 (m, 1H), 3.60-3.45 (m, 1H), 3.28-3.23 (m, 3H), 2.39-2.25 (m, 3H), 2.25-2.11 (m, 1H), 2.03-1.89 (m, 4H), 1.83-1.69 (m, |
| | (methylene) | | | 1H), 1.22-1.16 (m, |
| | bis(2,2- | | | 18H). |
| | dimethylpro- | | | |
| | panoate) | | | |
| 296 | ((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) | 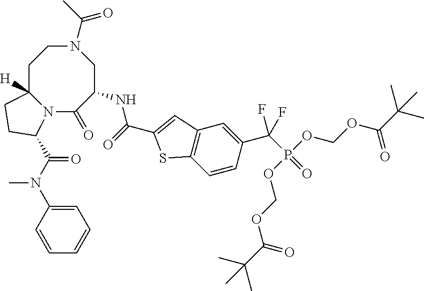 [SMILES: CC(=O)N1CC[C@H]2CC[C@@H](C(=O)N(C)c3ccccc3)N2C(=O)[C@@H](NC(=O)c2cc3cc(C(F)(F)P(=O)(OCOC(=O)C(C)(C)C)OCOC(=O)C(C)(C)C)ccc3s2)C1] | 877.3 [M + H]⁺ | ¹H NMR (400 MHz, METHANOL-d₄) δ 8.18-7.98 (m, 3H), 7.67-7.58 (m, 1H), 7.51-7.32 (m, 5H), 5.81-5.68 (m, 4H), 5.08-4.90 (m, 1H), 4.48-4.36 (m, 1H), 4.32-4.19 (m, 1H), 4.06-3.77 (m, 2H), 3.71-3.46 (m, 2H), 3.28-3.20 (m, 3H), 2.34-2.21 (m, 3H), 2.03 (s, 2H), 2.01-1.74 (m, 4H), 1.23-1.18 (m, 18H). |
| | bis(methylene) | | | |
| | bis(2,2- | | | |
| | dimethylpro- | | | |
| | panoate) | | | |
| 297 | ((((2- (((5S,8S, 10aR)- 3-acetyl- 8-((4- methoxy- phenyl) (methyl) carbamoyl)-6- oxodecahydro- pyrrolo[1,2- a][1,5] diazocin-5-yl) carbamoyl)- 1H-indol-5- yl)difluoro- methyl)phos- phoryl) bis(oxy)) | 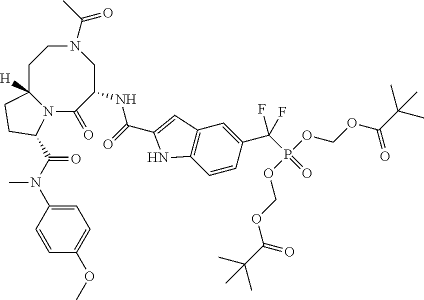 [SMILES: COc1ccc(N(C)C(=O)[C@@H]2CC[C@@H]3CCN(C(C)=O)C[C@H](NC(=O)c4cc5cc(C(F)(F)P(=O)(OCOC(=O)C(C)(C)C)OCOC(=O)C(C)(C)C)ccc5[nH]4)C(=O)N32)cc1] | 890.4 [M + H]⁺ | ¹H NMR (400 MHz, METHANOL-d₄) δ 7.94-7.84 (m, 1H), 7.59-7.24 (m, 5H), 7.01-6.87 (m, 2H), 5.77-5.62 (m, 4H), 5.09-4.94 (m, 1H), 4.46-4.19 (m, 2H), 4.03-3.98 (m, 1H), 3.89-3.83 (m, 1H), 3.81-3.71 (m, 3H), 3.68-3.46 (m, 2H), 3.23-3.19 (m, 2H), 2.35-2.14 (m, 3H), 2.10-1.71 (m, 7H), 1.19 (s, 18H). |
| | bis(methylene) | | | |
| | bis(2,2- | | | |
| | dimethylpro- | | | |
| | panoate) | | | |
| 298 | ((((2- (((5S,8S, 10aR)- 3-acetyl-8-((1- acetylazetidin- 3-yl) carbamoyl)-6- oxodecahydro- pyrrolo[1,2- a][1,5] diazocin-5- yl)carbamoyl)- 1H-indol-5- yl)difluoro- methyl)phos- phoryl) bis(oxy)) bis(methylene) | 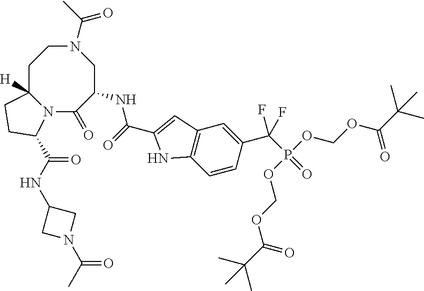 [SMILES: CC(=O)N1CC(NC(=O)[C@@H]2CC[C@@H]3CCN(C(C)=O)C[C@H](NC(=O)c4cc5cc(C(F)(F)P(=O)(OCOC(=O)C(C)(C)C)OCOC(=O)C(C)(C)C)ccc5[nH]4)C(=O)N32)C1] | 867.3 [M + H]⁺ | ¹H NMR (400 MHz, METHANOL-d₄) δ 7.91 (br. s., 1H), 7.59- 7.56 (m, 1H), 7.45- 7.42 (m, 1H), 7.34- 7.23 (m, 1H), 5.78- 5.62 (m, 4H), 5.19 5.08 (m, 1H), 4.59-4.34 (m, 4H), 4.27-4.24 (m, 1H), 4.14-3.54 (m, 6H), 2.35-2.10 (m, 6H), 2.08-1.95 (m, 2H), 1.90-1.86 (m, 4H), 1.19 (br. s., 18H). |
| | bis(2,2- | | | |
| | dimethylpro- | | | |
| | panoate) | | | |
| 299 | ((((2-(((5S, 8S,10aR)- 3-acetyl- 8-((R)-3-(N- methylpro- pionamido) pyrrolidine-1- carbonyl)-6- oxodecahydro- pyrrolo[1,2- a][1,5] diazocin-5- yl)carbamoyl)- 1H-indol-5- yl)difluoro- methyl)phos- phoryl) bis(oxy)) | 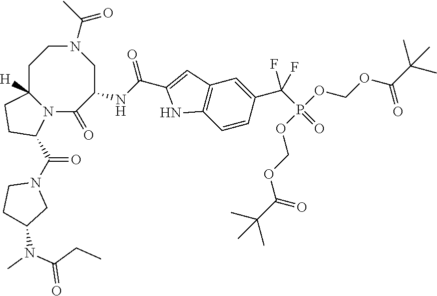 [SMILES: CCC(=O)N(C)[C@@H]1CCN(C(=O)[C@@H]2CC[C@@H]3CCN(C(C)=O)C[C@H](NC(=O)c4cc5cc(C(F)(F)P(=O)(OCOC(=O)C(C)(C)C)OCOC(=O)C(C)(C)C)ccc5[nH]4)C(=O)N32)C1] | 909.4 [M + H]⁺ | ¹H NMR (400 MHz, METHANOL-d₄) δ 7.91-7.89 (m, 1H), 7.60-7.51 (m, 1H), 7.46-7.38 (m, 1H), 7.33-7.22 (m, 1H), 5.76-5.63 (m, 4H), 5.27-4.99 (m, 2H), 4.79-4.64 (m, 1H), 4.49-4.32 (m, 1H), 4.20-3.95 (m, 1H), 3.89-3.33 (m, 7H), 3.03-2.81 (m, 3H), 2.56-2.34 (m, 3H), 2.31 (s, 2H), 2.28-2.13 (m, 3H), 2.12-1.82 (m, 5H), 1.20-1.16 (m, 18H), 1.15-1.03 (m, |
| | bis(methylene) | | | 3H). |
| | bis(2,2- | | | |
| | dimethylpro- | | | |
| | panoate) | | | |
| 300 | ((((2-(((5S, 8S,10aR)- 3-acetyl-8- (methyl (phenyl) carbamoyl)-6- oxodecahydro- pyrrolo[1,2- a][1,5] diazocin-5- yl)carbamoyl)- 1H-indol-5- yl)difluoro- methyl)phos- phoryl) bis(oxy)) | 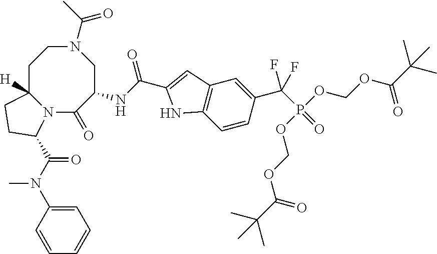 [SMILES: CC(=O)N1CC[C@H]2CC[C@@H](C(=O)N(C)c3ccccc3)N2C(=O)[C@@H](NC(=O)c2cc3cc(C(F)(F)P(=O)(OCOC(=O)C(C)(C)C)OCOC(=O)C(C)(C)C)ccc3[nH]2)C1] | 860.4 [M + H]⁺ | ¹H NMR (400 MHz, METHANOL-d₄) δ 7.94-7.86 (m, 1H), 7.59-7.34 (m, 7H), 7.32-7.25 (m, 1H), 5.77-5.63 (m, 4H), 5.10-4.94 (m, 1H), 4.44-4.14 (m, 2H), 4.05-3.79 (m, 2H), 3.72-3.43 (m, 2H), 3.27-3.24 (m, 2H), 2.36-2.25 (m, 3H), 2.07-1.86 (m, 6H), 1.83-1.69 (m, 1H), 1.21-1.17 (m, 18H). |
| | bis(methylene) | | | |
| | bis(2,2- | | | |
| | dimethylpro- | | | |
| | panoate) | | | |
| 301 | ((((2-(((5S, 8S,10aR)- 3-acetyl- 8-(methyl(3- propionamido- propyl) carbamoyl)-6- oxodecahydro- pyrrolo[1,2- a][1,5] diazocin-5- yl)carbamoyl)- 1H-indol-5- yl)difluoro- methyl)phos- phoryl)bis(oxy)) bis(methylene) bis(2,2- dimethylpro- panoate) | 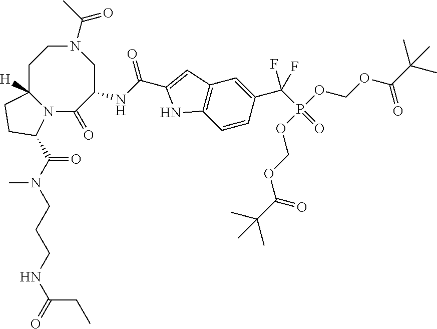 [SMILES: CCC(=O)NCCCN(C)C(=O)[C@@H]1CC[C@@H]2CCN(C(C)=O)C[C@H](NC(=O)c3cc4cc(C(F)(F)P(=O)(OCOC(=O)C(C)(C)C)OCOC(=O)C(C)(C)C)ccc4[nH]3)C(=O)N21] | 897.5 [M + H]⁺ | ¹H NMR (400 MHz, METHANOL-d₄) δ 7.90 (br. s., 1H), 7.59- 7.53 (m, 1H), 7.44- 7.40 (m, 1H), 7.33- 7.23 (m, 1H), 5.75- 5.63 (m, 4H), 5.16 4.91 (m, 1H), 4.51-4.34 (m, 1H), 4.05-3.78 (m, 2H), 3.75-3.37 (m, 3H), 3.27-3.04 (m, 4H), 2.96-2.90 (m, 1H), 2.46-2.36 (m, 1H), 2.35 2.29 (m, 2H), 2.28-2.12 (m, 4H), 2.11-1.98 (m, 3H), 1.98-1.68 (m, 5H), 1.19 (s, 18H), 1.14- 1.03 (m, 3H). |
| 302 | S-(2-((((7- (((5S,8S, 10aR)- 3-acetyl- 8-((3R,4S or 3S,4R)-3- cyano-4-(2- oxo-1,2- dihydro- pyridin-4-yl) pyrrolidine-1- carbonyl)-6- oxodecahydro- pyrrolo[1,2- a][1,5] diazocin-5- yl)carbamoyl) naphthalen-2- yl)difluoro- methyl)(((iso- propoxy- | 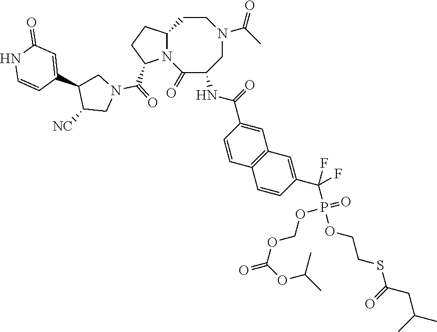 [SMILES: CC(=O)N1CC[C@H]2CC[C@@H](C(=O)N3C[C@H](c4cc[nH]c(=O)c4)[C@@H](C#N)C3)N2C(=O)[C@@H](NC(=O)c2ccc3ccc(C(F)(F)P(=O)(OCCSC(=O)CC(C)C)OCOC(=O)OC(C)C)cc3c2)C1] | 985.3 [M + H]⁺ | ¹H NMR (400 MHz, CDCl₃) δ 8.94-8.62 (m, 1H), 8.49-7.34 (m, 7H), 6.83-6.09 (m, 2H), 5.83-5.52 (m, 2H), 5.02-4.82 (m, 2H), 4.54-4.38 (m, 1H), 4.26-4.10 (m, 5H), 3.92-3.76 (m, 2H), 3.60-3.49 (m, 1H), 3.39-3.28 (m, 1H), 3.26-3.19 (m, 1H), 3.15-3.04 (m, 3H), 2.48-2.40 (m, 5H), 2.31-2.21 (m, 3H), 2.15-2.09 (m, 2H), 1.98-1.90 (m, 2H), 1.42 (t, J = 7.3 Hz, 2H), 1.35-1.27 (m, 6H), 0.95-0.88 (m, 6H). |
| | carbonyl)oxy) | or | | |
| | methoxy)phos- phoryl)oxy) ethyl)3- methylbutane- thioate | 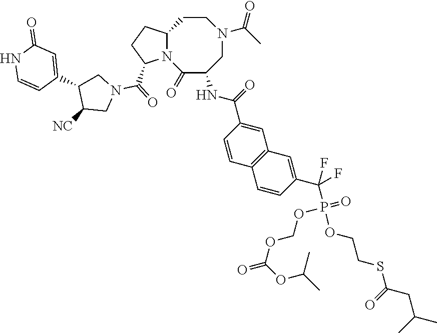 [SMILES: CC(=O)N1CC[C@H]2CC[C@@H](C(=O)N3C[C@@H](C#N)[C@H](c4cc[nH]c(=O)c4)C3)N2C(=O)[C@@H](NC(=O)c2ccc3ccc(C(F)(F)P(=O)(OCCSC(=O)CC(C)C)OCOC(=O)OC(C)C)cc3c2)C1] | | |
| | | SFC peak 1 was used for | | |
| | | biological testing | | |
| 303 | S-(2-((((7- (((5S,8S, 10aR)- 3-acetyl- 8-((3R,4S or 3S,4R)-3- cyano-4-(2- oxo-1,2- dihydro- pyridin- 4-yl) pyrrolidine-1- carbonyl)-6- oxodecahydro- pyrrolo[1,2- a][1,5] diazocin-5- yl)carbamoyl) naphthalen- 2-yl)difluoro- | 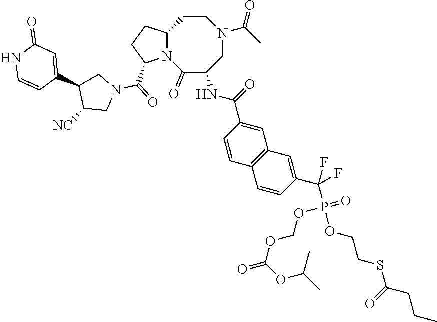 [SMILES: CCCC(=O)SCCOP(=O)(OCOC(=O)OC(C)C)C(F)(F)c1ccc2ccc(C(=O)N[C@H]3CN(C(C)=O)CC[C@H]4CC[C@@H](C(=O)N5C[C@H](c6cc[nH]c(=O)c6)[C@@H](C#N)C5)N4C3=O)cc2c1] | 971.3 [M + H]⁺ | ¹H NMR (400 MHz, DMSO-d₆) δ 8.85- 8.47 (m, 1H), 8.35- 8.24 (m, 1H), 8.19- 8.06 (m, 3H), 7.71 (d, J = 8.4 Hz, 1H), 7.43- 7.34 (m, 1H), 6.42- 6.33 (m, 1H), 6.30- 6.19 (m, 1H), 5.73- 5.64 (m, 2H), 5.12- 4.87 (m, 1H), 4.86- 4.73 (m, 1H), 4.67- 4.55 (m, 1H), 4.38- 4.13 (m, 4H), 4.04- 3.86 (m, 2H), 3.83- 3.67 (m, 2H), 3.67- 3.35 (m, 4H), 3.13 (t, J = 6.2 Hz, 2H), 2.53 (s, 3H), 2.36-2.25 (m, 1H), 2.23-2.16 (m, |
| | methyl)(((iso- | or | | 2H), 2.14-2.08 (m, |
| | propoxy- carbonyl) oxy)methoxy) phosphoryl) oxy)ethyl) butanethioate | 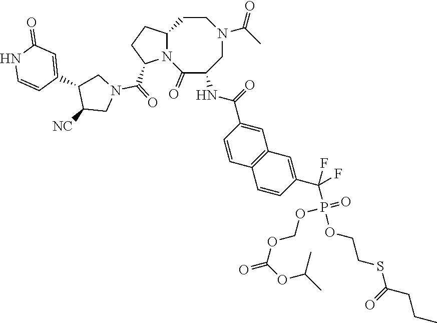 [SMILES: CCCC(=O)SCCOP(=O)(OCOC(=O)OC(C)C)C(F)(F)c1ccc2ccc(C(=O)N[C@H]3CN(C(C)=O)CC[C@H]4CC[C@@H](C(=O)N5C[C@@H](C#N)[C@H](c6cc[nH]c(=O)c6)C5)N4C3=O)cc2c1] | | 1H), 2.07-1.88 (m, 2H), 1.88-1.68 (m, 3H), 1.57-1.48 (m, 2H), 1.25-1.20 (m, 6H), 0.84 (t, J = 7.4 Hz, 3H). |
| | | SFC peak 1 was used for | | |
| | | biological testing | | |
| 304 | S,S′-(((((7- (((5S,8S, 10aR)- 3-acetyl- 8-((3R,4S or 3S,4R)-3- cyano-4-(2- oxo-1,2- dihydro- pyridin-4-yl) pyrrolidine-1- carbonyl)-6- oxodecahydro- pyrrolo[1,2- a][1,5] diazocin-5- | 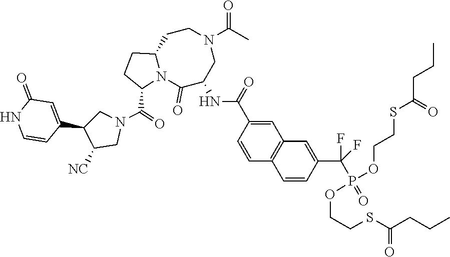 [SMILES: CCCC(=O)SCCOP(=O)(OCCSC(=O)CCC)C(F)(F)c1ccc2ccc(C(=O)N[C@H]3CN(C(C)=O)CC[C@H]4CC[C@@H](C(=O)N5C[C@H](c6cc[nH]c(=O)c6)[C@@H](C#N)C5)N4C3=O)cc2c1] | 985.4 [M + H]⁺ | ¹H NMR (400 MHz, DMSO) δ 11.62 (s, 1H), 8.84-8.49 (m, 2H), 8.36-8.26 (m, 1H), 8.20-8.04 (m, 3H), 7.77-7.69 (m, 1H), 7.44-7.35 (m, 1H), 6.42-6.32 (m, 1H), 6.32-6.19 (m, 1H), 5.10-4.89 (m, 1H), 4.70-4.57 (m, 1H), 4.38-4.12 (m, 6H), 4.01-3.85 (m, 2H), 3.84-3.69 (m, 2H), 3.68-3.36 (m, 4H), 3.23-3.09 (m, |
| | yl)carbamoyl) | or | | 5H), 2.56-2.51 (m, |
| | naphthalen-2- yl)difluoro- methyl)phos- phoryl) bis(oxy)) bis(ethane- 2,1-diyl))di- butanethioate | 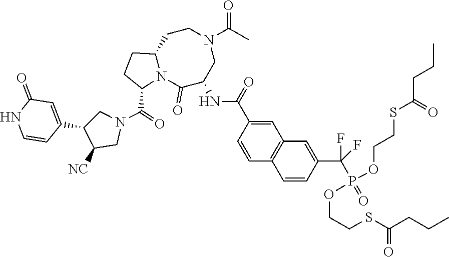 [SMILES: CCCC(=O)SCCOP(=O)(OCCSC(=O)CCC)C(F)(F)c1ccc2ccc(C(=O)N[C@H]3CN(C(C)=O)CC[C@H]4CC[C@@H](C(=O)N5C[C@@H](C#N)[C@H](c6cc[nH]c(=O)c6)C5)N4C3=O)cc2c1] | | 4H), 2.49-2.24 (m, 2H), 2.22-2.16 (m, 2H), 2.13-2.07 (m, 1H), 2.07-1.87 (m, 2H), 1.87-1.68 (m, 3H), 1.59-1.49 (m, 4H), 0.85 (t, J = 7.4 Hz, 6H). |
| | | SFC peak 1 was used for | | |
| | | biological testing | | |
| 305 | S,S′-(((((7- (((5S,8S, 10aR)- 3-acetyl- 8-((3S,4S)-3- hydroxy-4- (1H-1,2,3- triazol-1-yl) pyrrolidine-1- carbonyl)-6- oxodecahydro- pyrrolo[1,2- a][1,5] diazocin-5- yl)carbamoyl) naphthalen-2- yl)difluoro- methyl)phos- phoryl) bis(oxy)) bis(ethane- 2,1-diyl)) bis(3- methylbutane- thioate) | 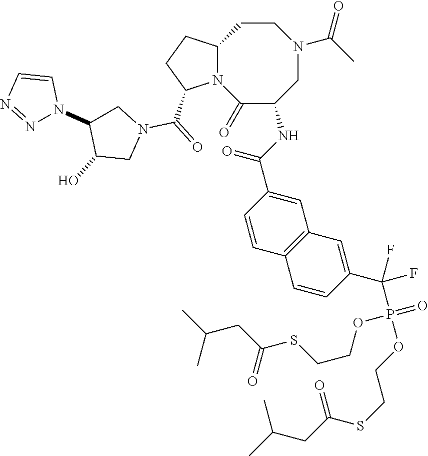 [SMILES: CC(=O)N1CC[C@H]2CC[C@@H](C(=O)N3C[C@H](O)[C@@H](n4ccnn4)C3)N2C(=O)[C@@H](NC(=O)c2ccc3ccc(C(F)(F)P(=O)(OCCSC(=O)CC(C)C)OCCSC(=O)CC(C)C)cc3c2)C1] | 978.3 [M + H]⁺ | ¹H NMR (400 MHz, DMSO) δ 8.74-8.68 (m, 1H), 8.58-8.48 (m, 1H), 8.37-8.27 (m, 1H), 8.22-8.14 (m, 2H), 8.14-8.07 (m, 2H), 7.81-7.75 (m, 1H), 7.75-7.70 (m, 1H), 6.16-5.69 (m, 1H), 5.15-4.90 (m, 2H), 4.69-4.44 (m, 2H), 4.35-4.27 (m, 1H), 4.23-4.13 (m, 4H), 4.04-3.85 (m, 2H), 3.83-3.59 (m, 3H), 3.54-3.47 (m, 3H), 3.17-3.10 (m, 4H), 2.45-2.39 (m, 4H), 2.38-2.28 (m, 1H), 2.23-2.18 (m, 2H), 2.14-2.10 (m, 1H), 2.07-1.90 (m, 4H), 1.89-1.67 (m, 3H), 0.86 (d, J = 6.6 Hz, 12H). |
| 306 | S,S′-(((((7- (((5S,8S, 10aR)- 3-acetyl- 8-((3S,4R or 3r,4S)-3- cyano-4-(2- oxo-1,2- dihydro- pyridin- 4-yl) pyrrolidine-1- carbonyl)-6- oxodecahydro- pyrrolo[1,2- a][1,5] | 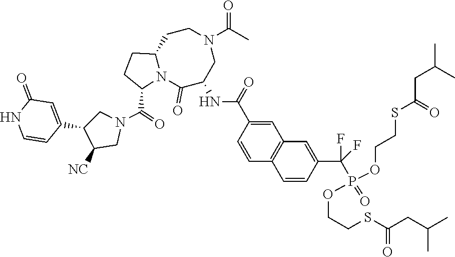 [SMILES: CC(=O)N1CC[C@H]2CC[C@@H](C(=O)N3C[C@@H](C#N)[C@H](c4cc[nH]c(=O)c4)C3)N2C(=O)[C@@H](NC(=O)c2ccc3ccc(C(F)(F)P(=O)(OCCSC(=O)CC(C)C)OCCSC(=O)CC(C)C)cc3c2)C1] | 1013 [M + H]⁺ | ¹H NMR (400 MHz, CDCl₃) δ 8.72-8.63 (m, 1H), 8.57-8.45 (m, 1H), 8.25-8.08 (m, 2H), 8.02-7.69 (m, 4H), 7.59-7.41 (m, 1H), 7.27-7.13 (m, 1H), 6.50-6.35 (m, 1H), 4.98-4.81 (m, 1H), 4.74-4.60 (m, 1H), 4.54-4.40 (m, 1H), 4.27-4.13 (m, 7H), 4.08-3.98 (m, 1H), 3.87-3.66 (m, 2H), 3.37-3.24 (m, 2H), 3.22-3.05 |
| | diazocin-5- | or | | (m, 6H), 2.47 (s, 3H), |
| | yl)carbamoyl) naphthalen-2- yl)difluoro- methyl)phos- phoryl) bis(oxy)) bis(ethane- 2,1-diyl)) bis(3- methylbutane- thioate) | 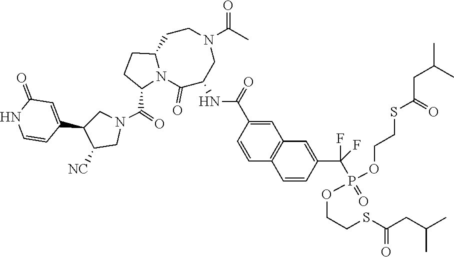 [SMILES: CC(=O)N1CC[C@H]2CC[C@@H](C(=O)N3C[C@H](c4cc[nH]c(=O)c4)[C@@H](C#N)C3)N2C(=O)[C@@H](NC(=O)c2ccc3ccc(C(F)(F)P(=O)(OCCSC(=O)CC(C)C)OCCSC(=O)CC(C)C)cc3c2)C1] | | 2.45-2.40 (m, 4H), 2.30-2.07 (m, 6H), 1.99-1.87 (m, 2H), 0.99-0.89 (m, 12H). |
| | | SFC peak 2 was used for | | |
| | | biological testing | | |
| 307 | (((2-(((5S, 8S,10aR)- 3-acetyl-8- (methyl (phenyl) carbamoyl)- 6-oxo- decahydro- pyrrolo[1,2- a][1,5] diazocin-5- yl)carbamoyl) benzo[b] thiophene-5- carbonyl) phosphoryl) bis(oxy))bis (methylene) bis(2,2- dimethylpro- panoate) | 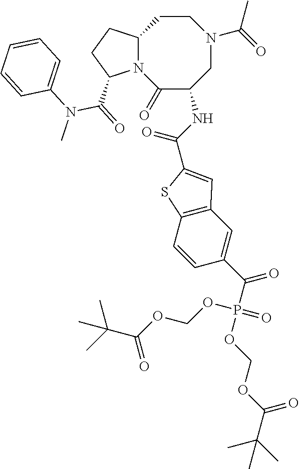 [SMILES: CC(=O)N1CC[C@H]2CC[C@@H](C(=O)N(C)c3ccccc3)N2C(=O)[C@@H](NC(=O)c2cc3cc(C(=O)P(=O)(OCOC(=O)C(C)(C)C)OCOC(=O)C(C)(C)C)ccc3s2)C1] | 855.2 [M + H]⁺ | ¹H NMR (400 MHz, CDCl₃) δ 8.92 (s, 1H), 8.17-8.08 (m, 1H), 7.93-7.86 (m, 2H), 7.75-7.72 (m, 1H), 7.44-7.39 (m, 2H), 7.36-7.30 (m, 3H), 5.83-5.67 (m, 4H), 4.68-4.62 (m, 1H), 4.39-4.33 (m, 1H), 4.16-4.08 (m, 2H), 4.03 (s, 1H), 3.34- 3.20 (m, 5H), 2.38 (s, 3H), 2.01-1.92 (m, 2H), 1.84-1.70 (m, 4H), 1.07 (s, 18H). |
| 308 | ((((2-(((5S, 8S,10aR)- 8-([1,1′- biphenyl]-4- ylcarbamoyl)- 3-acetyl-6- oxodecahydro- pyrrolo[1,2- a][1,5] diazocin-5- yl)carbamoyl)- 1H-indol-5- yl)difluoro- methyl)phos- phoryl) bis(oxy)) bis(methylene) bis(2,2- dimethylpro- panoate) | 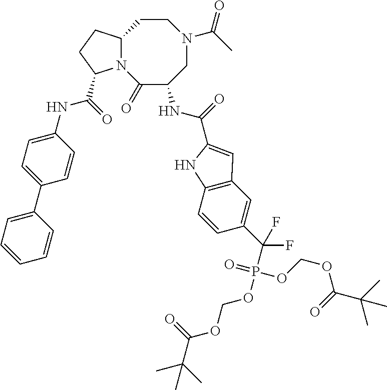 [SMILES: CC(=O)N1CC[C@H]2CC[C@@H](C(=O)Nc3ccc(-c4ccccc4)cc3)N2C(=O)[C@@H](NC(=O)c2cc3cc(C(F)(F)P(=O)(OCOC(=O)C(C)(C)C)OCOC(=O)C(C)(C)C)ccc3[nH]2)C1] | 922.4 [M + H]⁺ | ¹H NMR (400 MHz, CDCl₃) δ 9.85-9.48 (m, 1H), 8.85-8.44 (m, 1H), 8.16-7.75 (m, 2H), 7.67-7.30 (m, 11H), 7.09-6.88 (m, 1H), 5.81-5.59 (m, 4H), 5.27-4.92 (m, 1H), 4.82-4.60 (m, 1H), 4.37-3.80 (m, 3H), 3.61-3.02 (m, 2H), 2.53-2.41 (m, 1H), 2.29-2.21 (m, 3H), 1.92-1.81 (m, 4H), 1.21 (s, 18H). |
| 309 | ((((2-(((5S, 8S,10aR)- 3-acetyl- 8-((2- (methyl- sulfonyl) pyrimidin-5- yl)carbamoyl)- 6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- panoate) | 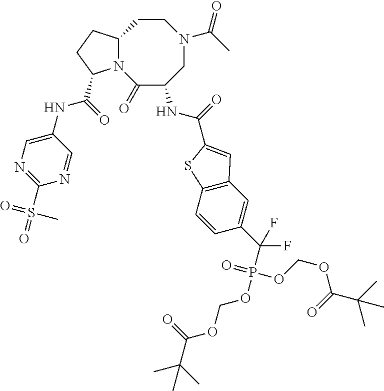 [SMILES: CC(=O)N1CC[C@H]2CC[C@@H](C(=O)Nc3cnc(S(C)(=O)=O)nc3)N2C(=O)[C@@H](NC(=O)c2cc3cc(C(F)(F)P(=O)(OCOC(=O)C(C)(C)C)OCOC(=O)C(C)(C)C)ccc3s2)C1] | 942.0 [M + H]⁺ | ¹H NMR (400 MHz, CDCl₃) δ 9.74 (s, 1H), 9.17 (s, 2H), 8.09 (s, 1H), 7.93 (m, 1H), 7.88 (s, 1H), 7.62 (m, 2H), 5.77-5.61 (m, 4H), 4.97 (s, 1H), 4.82 (s, 1H), 4.74 (s, 1H), 4.42 (s, 1H), 4.25 (s, 1H), 4.13 (m, 1H), 3.95 (s, 1H), 3.47 (s, 1H), 3.31 (m, 4H), 3.01 (s, 1H), 2.33 (s, 3H), 2.14 (s, 1H), 1.99 (s, 2H), 1.18 (s, 18H). |
| 310 | ((((2-(((5S, 8S,10aR)- 8-([1,1′- biphenyl]-4- yl(methyl) carbamoyl)-3- acetyl-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- panoate) | 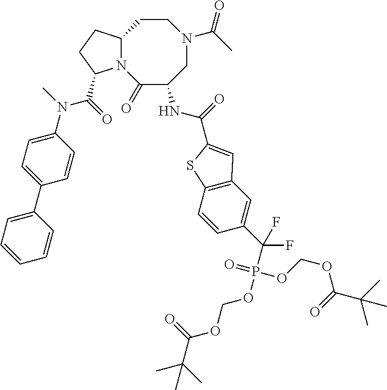 [SMILES: CC(=O)N1CC[C@H]2CC[C@@H](C(=O)N(C)c3ccc(-c4ccccc4)cc3)N2C(=O)[C@@H](NC(=O)c2cc3cc(C(F)(F)P(=O)(OCOC(=O)C(C)(C)C)OCOC(=O)C(C)(C)C)ccc3s2)C1] | 953.0 [M + H]⁺ | ¹H NMR (400 MHz, CDCl₃) δ 8.10 (s, 1H), 7.94 (d, J = 8.6 Hz, 1H), 7.85 (s, 1H), 7.75 (d, J = 5.4 Hz, 1H), 7.65 (dd, J = 14.6, 8.4 Hz, 3H), 7.57 (d, J = 7.7 Hz, 2H), 7.51-7.39 (m, 4H), 7.35 (t, J = 7.1 Hz, 1H), 5.72 (dt, J = 12.6, 4.4 Hz, 2H), 5.64 (dt, J = 11.8, 5.7 Hz, 2H), 4.71 (s, 1H), 4.50 (t, J = 8.9 Hz, 1H), 4.19 (d, J = 14.5 Hz, 2H), 4.09 (s, 1H), 3.51- 3.39 (m, 1H), 3.35 (s, 3H), 3.25 (t, J = 12.5 Hz, 1H), 2.43 (s, 3H), 2.30 (m, 1H), 2.03 (s, 1H), 2.01-1.93 (m, 1H), 1.83 (m, 2H), 1.76 (s, 1H), 1.19 (s, 18H). |
| 311 | ((((2-(((5S, 8S,10aR)- 3-acetyl- 8-(methyl(4- morpholino- phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- panoate) | 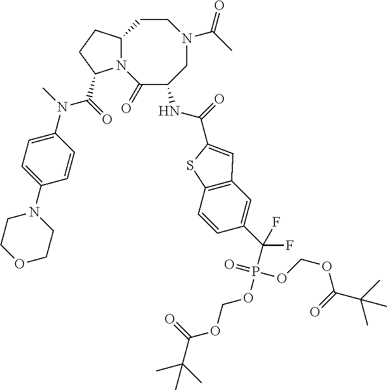 [SMILES: CC(=O)N1CC[C@H]2CC[C@@H](C(=O)N(C)c3ccc(N4CCOCC4)cc3)N2C(=O)[C@@H](NC(=O)c2cc3cc(C(F)(F)P(=O)(OCOC(=O)C(C)(C)C)OCOC(=O)C(C)(C)C)ccc3s2)C1] | 963.0 [M + H]⁺ | ¹H NMR (400 MHz, CDCl₃) δ 8.10 (s, 1H), 7.94 (d, J = 8.5 Hz, 1H), 7.84 (s, 1H), 7.74 (d, J = 5.8 Hz, 1H), 7.63 (d, J = 8.5 Hz, 1H), 7.30 (s, 2H), 7.03 (s, 2H), 5.76-5.69 (m, 2H), 5.65 (t, J = 13.4 Hz, 2H), 4.69 (s, 1H), 4.42 (d, J = 8.9 Hz, 1H), 4.18 (d, J = 13.8 Hz, 2H), 4.06 (s, 1H), 3.88 (s, 4H), 3.38 (s, 1H), 3.28 (s, 3H), 3.20 (s, 5H), 2.42 (d, J = 1.8 Hz, 3H), 1.98 (s, 2H), 1.81 (d, J = 14.2 Hz, 2H), 1.74 (s, 2H), 1.19 (s, 18H). |
| 312 | ((((2-(((5S, 8S,10aR)- 3-acetyl- 8-(methyl(1- (methyl- sulfonyl) piperidin-4- yl)carbamoyl)- 6- oxodecahydro- pyrrolo[1,2- a][1,5] diazocin-5- yl)carbamoyl) benzo[b] thiophen-5- yl)difluoro- methyl)phos- phoryl) bis(oxy))bis (methylene) bis(2,2- dimethylpro- panoate) | 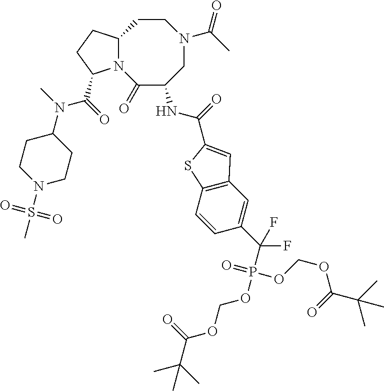 [SMILES: CC(=O)N1CC[C@H]2CC[C@@H](C(=O)N(C)C3CCN(S(C)(=O)=O)CC3)N2C(=O)[C@@H](NC(=O)c2cc3cc(C(F)(F)P(=O)(OCOC(=O)C(C)(C)C)OCOC(=O)C(C)(C)C)ccc3s2)C1] | 962.2 [M + H]⁺ | ¹H NMR (600 MHz, CDCl₃) δ 8.08 (s, 1H), 7.95 (d, J = 8.7 Hz, 1H), 7.84 (s, 1H), 7.70 (d, J = 5.8 Hz, 1H), 7.65 (d, J = 8.6 Hz, 1H), 5.73 (m, 2H), 5.68-5.64 (m, 2H), 4.79 (m, 2H), 4.61 (m, 1H), 4.23-4.13 (m, 3H), 3.94 (m, 2H), 3.36 (m, 1H), 3.25 (m, 1H), 3.01 (s, 3H), 2.80 (s, 3H), 2.79-2.72 (m, 2H), 2.46 (s, 3H), 2.41- 2.33 (m, 2H), 2.13- 2.07 (m, 1H), 2.03- 1.96 (m, 1H), 1.90- 1.79 (m, 8H), 1.20 (s, 18H). |
| 313 | ((((2-(((5S, 8S,10aR)- 3-acetyl- 8-((1- (methyl- sulfonyl) piperidin-4- yl)carbamoyl)- 6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- panoate) | 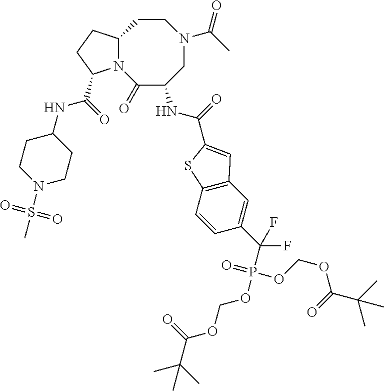 [SMILES: CC(=O)N1CC[C@H]2CC[C@@H](C(=O)NC3CCN(S(C)(=O)=O)CC3)N2C(=O)[C@@H](NC(=O)c2cc3cc(C(F)(F)P(=O)(OCOC(=O)C(C)(C)C)OCOC(=O)C(C)(C)C)ccc3s2)C1] | 948.0 [M + H]⁺ | ¹H NMR (400 MHz, CDCl₃) δ 8.09 (s, 1H), 7.94 (d, J = 8.5 Hz, 1H), 7.86 (s, 1H), 7.64 (d, J = 9.0 Hz, 2H), 6.35 (d, J = 8.0 Hz, 1H), 5.72 (dd, J = 12.7, 5.0 Hz, 2H), 5.68- 5.62 (m, 2H), 4.83 (s, 1H), 4.42 (t, J = 8.4 Hz, 1H), 4.22-4.06 (m, 3H), 3.90 (s, 1H), 3.81 (d, J = 12.1 Hz, 2H), 3.22-3.05 (m, 1H), 2.79 (s, 6H), 2.42 (s, 3H), 2.40-2.26 (m, 2H), 2.19 (d, J = 9.0 Hz, 1H), 2.07 (t, J = 15.6 Hz, 4H), 1.90- 1.82 (m, 1H), 1.61 (s, 2H), 1.19 (s, 18H). |
| 314 | ((((2-(((5S, 8S,10aR)- 3-acetyl- 8-((4- (methyl- sulfonyl) phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy))bis (methylene) bis(2,2- dimethylpro- panoate) | 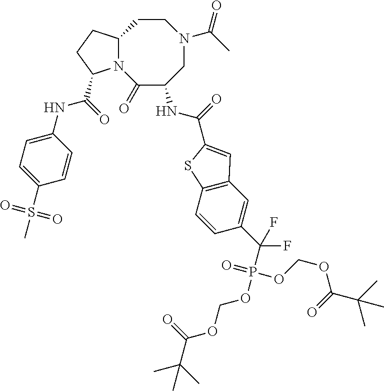 [SMILES: CC(=O)N1CC[C@H]2CC[C@@H](C(=O)Nc3ccc(S(C)(=O)=O)cc3)N2C(=O)[C@@H](NC(=O)c2cc3cc(C(F)(F)P(=O)(OCOC(=O)C(C)(C)C)OCOC(=O)C(C)(C)C)ccc3s2)C1] | 939.0 [M − H]⁻ | ¹H NMR (600 MHz, DMSO-d₆) δ 10.61 (m, 1H), 9.13-8.64 (m, 1H), 8.31-8.23 (m, 1H), 8.22-7.97 (m, 2H), 7.86-7.77 (m, 4H), 7.63-7.53 (m, 1H), 5.71-5.38 (m, 2H), 5.07-4.85 (m, 1H), 4.58 (s, 1H), 4.53- 4.47 (m, 1H), 4.30 (s, 1H), 3.80 (m, 1H), 3.69-3.63 (m, 1H), 3.22-3.17 (m, 2H), 3.14 (m, 3H), 3.13 (s, 1H), 2.27 (m, 1H), 2.11 (s, 3H), 2.08 (m, 1H), 1.97 (s, 2H), 1.84-1.73 (m, 2H), 1.12 (s, 9H), 1.09 (s, 9H). |
| 315 | ((((4-((E)-3- (((5S,8S, 10aR)- 3-acetyl-8- (methyl (phenyl) carbamoyl)-6- oxodecahydro- pyrrolo[1,2- a][1,5] diazocin-5- yl)amino)-3- oxoprop-1- en-1- yl)phenyl) difluoro- methyl) phosphoryl) bis(oxy))bis (methylene) bis(2,2- dimethylpro- panoate) | 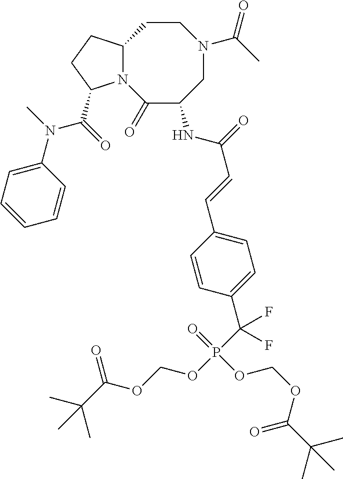 [SMILES: CC(=O)N1CC[C@H]2CC[C@@H](C(=O)N(C)c3ccccc3)N2C(=O)[C@@H](NC(=O)/C=C/c2ccc(C(F)(F)P(=O)(OCOC(=O)C(C)(C)C)OCOC(=O)C(C)(C)C)cc2)C1] | | ¹H NMR (600 MHz, CDCl₃) δ 7.71-7.56 (m, 5H), 7.47 (m, 2H), 7.39 (m, 3H), 7.16 (d, J = 5.8 Hz, 1H), 6.51 (m, 1H), 5.78-5.71 (m, 2H), 5.65 (m, 2H), 4.67 (s, 1H), 4.40 (m, 1H), 4.23-4.02 (m, 3H), 3.39 (m, 1H), 3.32 (s, 3H), 3.18 (m, 1H), 2.44 (s, 3H), 2.32 (m, 1H), 2.03 (m, 1H), 1.91- 1.87 (m, 3H), 1.73 (m, 1H), 1.23-1.20 (s, 18H). |
| 316 | ((((2- (((5S,8S, 10aR)- 3-acetyl- 8-((4- cyanophenyl) (methyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- panoate) | 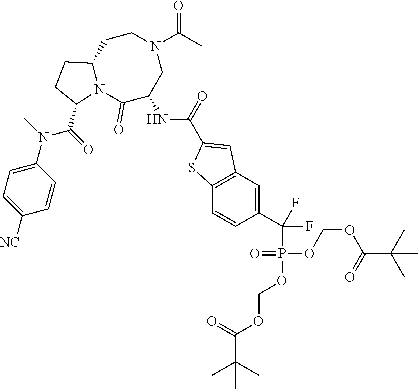 [SMILES: CC(=O)N1CC[C@H]2CC[C@@H](C(=O)N(C)c3ccc(C#N)cc3)N2C(=O)[C@@H](NC(=O)c2cc3cc(C(F)(F)P(=O)(OCOC(=O)C(C)(C)C)OCOC(=O)C(C)(C)C)ccc3s2)C1] | 903.0 [M + H]⁺ | ¹H NMR (400 MHz, CDCl₃) δ 8.10 (s, 1H), 7.94 (d, J = 8.5 Hz, 1H), 7.85 (s, 1H), 7.78 (d, J = 8.1 Hz, 2H), 7.64 (m, 1H), 7.57 (m, 2H), 7.49 (s, 1H), 5.73 (m, 1H), 5.70 (m, 1H), 5.66 (m, 1H), 5.64- 5.61 (m, 1H), 4.97 (s, 1H), 4.30 (m, 2H), 4.12 (m, 1H), 3.76 (s, 1H), 3.28 (s, 3H), 3.20 (s, 1H), 3.00 (s, 1H), 2.50- 2.43 (m, 1H), 2.27 (s, 3H), 2.05 (s, 3H), 1.88 (s, 1H), 1.57 (s, 1H), 1.18 (s, 18H). |
| 317 | diethyl ((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phonate | 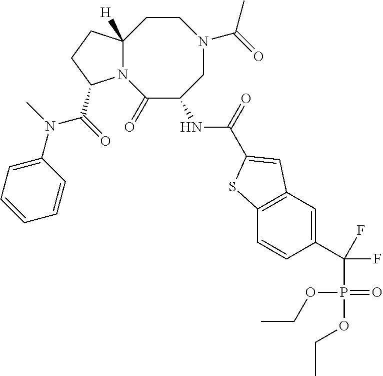 [SMILES: CCOP(=O)(OCC)C(F)(F)c1ccc2sc(C(=O)N[C@H]3CN(C(C)=O)CC[C@H]4CC[C@@H](C(=O)N(C)c5ccccc5)N4C3=O)cc2c1] | 705.4 [M + H]⁺ | ¹HNMR (400 MHz, METHANOL-d₄) δ 8.15 (s, 1H), 8.09-8.03 (m, 1H), 8.01-7.92 (m, 1H), 7.67-7.60 (m, 1H), 7.48-7.32 (m, 5H), 5.06 (dd, J = 3.6, 10.8 Hz, 1H), 4.31- 4.16 (m, 5H), 4.01 (dd, J = 3.6, 14 Hz, 1H), 3.92-3.80 (m, 1H), 3.69-3.52 (m, 2H), 3.26 (s, 2H), 3.23 (s, 1H), 2.32 (s, 2H), 2.26 (s, 1H), 2.15 (s, 1H), 2.08-1.87 (m, 5H), 1.86-1.68 (m, 1H), 1.35-1.29 (m, 6H) |
| 318 | ((((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) diisopropyl bis(carbonate) | 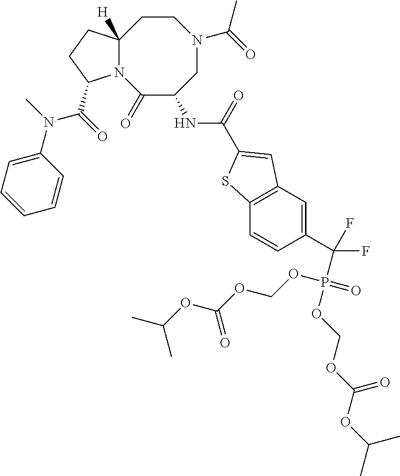 [SMILES: CC(=O)N1CC[C@H]2CC[C@@H](C(=O)N(C)c3ccccc3)N2C(=O)[C@@H](NC(=O)c2cc3cc(C(F)(F)P(=O)(OCOC(=O)OC(C)C)OCOC(=O)OC(C)C)ccc3s2)C1] | 881.2 [M + H]⁺ | ¹H NMR (400 MHz, METHANOL-d₄) δ 8.20-7.97 (m, 3H), 7.67-7.60 (m, 1H), 7.52-7.35 (m, 5H), 5.78-5.67 (m, 4H), 5.08-5.01 (m, 1H), 4.98-4.86 (m, 2H), 4.49-4.33 (m, 1H), 4.33-4.19 (m, 1H), 4.06-3.97 (m, 1H), 3.92-3.80 (m, 1H), 3.70-3.60 (m, 1H), 3.59-3.50 (m, 1H), 3.28-3.23 (m, 3H), 2.34-2.24 (m, 3H), 2.09-1.89 (m, 5H), 1.79-1.69 (m, 1H), 1.30-1.27 (m, 12H) |
| 319 | ((((2- (((5S,8S, 10aR)- 3-acetyl- 8-((4-(tert- butyl)phenyl) (methyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- panoate) | 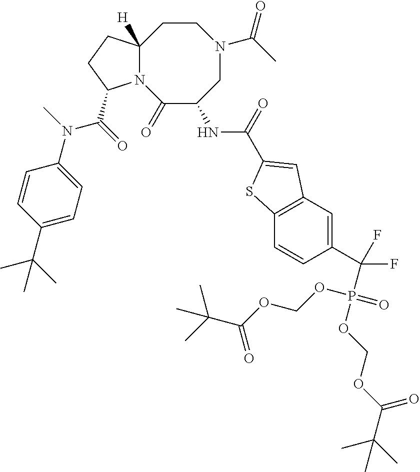 [SMILES: CC(=O)N1CC[C@H]2CC[C@@H](C(=O)N(C)c3ccc(C(C)(C)C)cc3)N2C(=O)[C@@H](NC(=O)c2cc3cc(C(F)(F)P(=O)(OCOC(=O)C(C)(C)C)OCOC(=O)C(C)(C)C)ccc3s2)C1] | 933.7 [M + H]⁺ | ¹H NMR (400 MHz, CHLOROFORM-d) δ 8.13 (s, 1H), 7.97 (d, J = 8.8 Hz, 1H), 7.87 (s, 1H), 7.78 (d, J = 4.8 Hz, 1H), 7.71-7.65 (m, 1H), 7.48 (d, J = 8.8 Hz, 2H), 7.37-7.29 (m, 2H), 5.78-5.65 (m, 4H), 4.80-4.63 (m, 1H), 4.53-4.37 (m, 1H), 4.28-4.07 (m, 3H), 3.44-3.22 (m, 5H), 2.45 (s, 3H), 2.11- 1.77 (m, 6H), 1.34 (s, 9H), 1.22 (d, J = 2.0 Hz, 18H) |
| 320 | ((((2- (((5S,8S, 10aR)- 3-acetyl- 8-((S)-2- benzyl- pyrrolidine-1- carbonyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) bis(2,2- dimethylpro- panoate) | 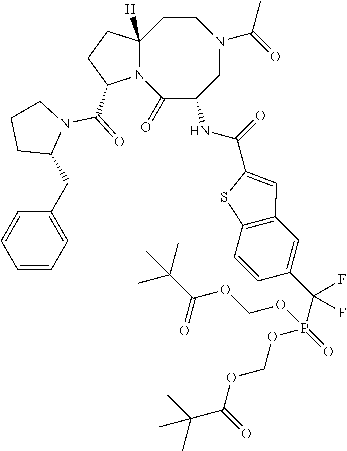 [SMILES: CC(=O)N1CC[C@H]2CC[C@@H](C(=O)N3CCC[C@H]3Cc3ccccc3)N2C(=O)[C@@H](NC(=O)c2cc3cc(C(F)(F)P(=O)(OCOC(=O)C(C)(C)C)OCOC(=O)C(C)(C)C)ccc3s2)C1] | 931.3 [M + H]⁺ | ¹H NMR (400 MHz, CHLOROFORM-d) δ 8.10 (s, 1H), 7.97 (d, J = 8.4 Hz, 1H), 7.87- 7.84 (m, 1H), 7.78 (d, J = 6.0 Hz, 1H), 7.67 (d, J = 8.8 Hz, 1H), 7.37-7.30 (m, 1H), 7.24 (t, J = 8.4 Hz, 3H), 5.78-5.64 (m, 4H), 4.86-4.76 (m, 1H), 4.69 (t, J = 8.4 Hz, 1H), 4.60-4.49 (m, 1H), 4.31-4.16 (m, 3H), 3.82 (d, J = 7.6 Hz, 1H), 3.57-3.35 (m, 2H), 3.30 (dd, J = 10.4, 14.4 Hz, 1H), 3.12 (dd, J = 3.6, 13.2 Hz, 1H), 2.69 (dd, J = 8.8, 13.2 Hz, 1H), 2.49 (s, 3H), 2.45-2.33 (m, 2H), 2.22-2.05 (m, 2H), 1.99-1.79 (m, 6H), 1.24-1.19 (m, 18H) |
| 321 | ((((7- (((5S,8S, 10aR)- 3-acetyl-8- (methyl (phenyl) carbamoyl)-6- oxodecahydro- pyrrolo[1,2- a][1,5] diazocin-5- yl)carbamoyl) naphthalen- 2- yl)difluoro- methyl)phos- phoryl) bis(oxy)) bis(methylene) bis(2,2- dimethylpro- panoate) | 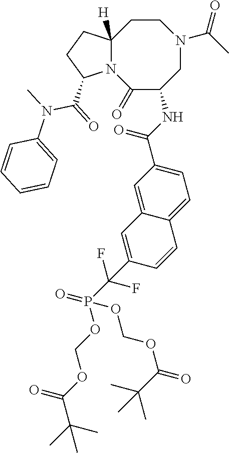 [SMILES: CC(=O)N1CC[C@H]2CC[C@@H](C(=O)N(C)c3ccccc3)N2C(=O)[C@@H](NC(=O)c2ccc3ccc(C(F)(F)P(=O)(OCOC(=O)C(C)(C)C)OCOC(=O)C(C)(C)C)cc3c2)C1] | 871.6 [M + H]⁺ | ¹H NMR (400 MHz, METHANOL-d₄) δ 8.69-8.50 (m, 1H), 8.34-8.16 (m, 1H), 8.16-8.00 (m, 3H), 7.82-7.70 (m, 1H), 7.54-7.36 (m, 5H), 5.85-5.68 (m,4H), 5.16-4.98 (m, 1H), 4.51-4.35 (m, 1H), 4.34-4.16 (m, 1H), 4.05 (dd, J = 3.6, 14.0 Hz, 1H), 3.93-3.82 (m, 1H), 3.78-3.47 (m, 2H), 3.29-3.24 (m, 3H),2.41-2.25 (m, 3H), 2.08-1.90 (m, 4H), 1.75 (d, J = 4.3 Hz, 1H), 1.39-1.26 (m, 1H), 1.18 (s, 18H) |
| 322 | ((((6- (((5S,8S, 10aR)- 3-acetyl-8- (methyl (phenyl) carbamoyl)-6- oxodecahydro- pyrrolo[1,2- a][1,5] diazocin-5- yl)carbamoyl) naphthalen-2- yl)difluoro- methyl)phos- phoryl) bis(oxy)) bis(methylene) bis(2,2- dimethylpro- panoate) | 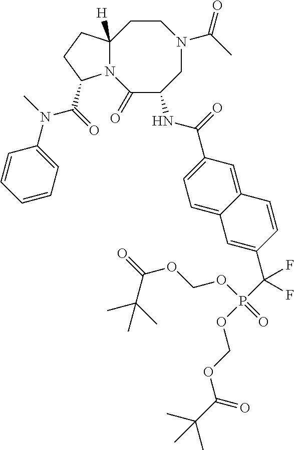 [SMILES: CC(=O)N1CC[C@H]2CC[C@@H](C(=O)N(C)c3ccccc3)N2C(=O)[C@@H](NC(=O)c2ccc3cc(C(F)(F)P(=O)(OCOC(=O)C(C)(C)C)OCOC(=O)C(C)(C)C)ccc3c2)C1] | 871.3 [M + H]⁺ | ¹H NMR (400 MHz, CHLOROFORM-d) δ 8.40 (s, 1H), 8.18 (s, 1H), 8.06-8.00 (m, 2H), 7.99-7.93 (m, 1H), 7.90 (d, J = 5.6 Hz, 1H), 7.75 (d, J = 8.4 Hz, 1H), 7.51-7.44 (m, 2H), 7.44-7.36 (m, 3H), 5.80-5.72 (m, 2H), 5.71-5.64 (m, 2H), 4.86-4.76 (m, 1H), 4.45 (t, J = 8.8 Hz, 1H), 4.28-4.09 (m, 3H), 3.51-3.40 (m, 1H), 3.35 (s, 3H), 3.33-3.24 (m, 1H), 2.49 (s, 3H), 2.41-2.30 (m, 1H), 2.11-1.97 (m, 1H), 1.96-1.71 (m, 4H), 1.21 (d, J = 1.3 Hz, 18H) |
| 323 | ((((7- (((5S,8S, 10aR)- 3-acetyl- 8-((rel- trans)-3- cyano-4- phenyl- pyrrolidine-1- carbonyl)-6- oxodecahydro- pyrrolo[1,2- a][1,5] diazocin-5- yl)carbamoyl) naphthalen-2- yl)difluoro- methyl)phos- phoryl) bis(oxy)) bis(methylene) bis(2,2- dimethylpro- panoate) | 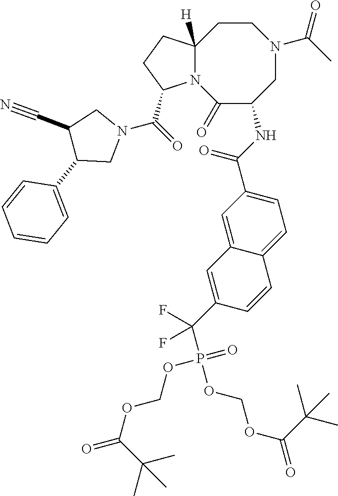 [SMILES: CC(=O)N1CC[C@H]2CC[C@@H](C(=O)N3C[C@H](c4ccccc4)[C@@H](C#N)C3)N2C(=O)[C@@H](NC(=O)c2ccc3ccc(C(F)(F)P(=O)(OCOC(=O)C(C)(C)C)OCOC(=O)C(C)(C)C)cc3c2)C1] | 936.3 [M + H]⁺ | ¹H NMR (400 MHz, METHANOL-d₄) δ 8.58 (s, 1H), 8.29 (s, 1H), 8.14-8.04 (m, 3H), 7.81-7.68 (m, 1H), 7.50-7.35 (m, 5H), 5.83-5.66 (m, 4H), 5.20-5.08 (m, 1H), 4.72 (dd, J = 7.8, 16.4 Hz, 1H), 4.58- 4.36 (m, 2H), 4.14- 4.02 (m, 2H), 3.90- 3.74 (m, 3H), 3.69- 3.51 (m, 4H), 2.43 (s, 4H), 2.26 (d, J = 6.8 Hz, 1H), 2.11-1.90 (m, 4H), 1.21-1.15 (m, 18H) |
| 324 | ((((4-((E)-3- (((5S,8S, 10aR)- 8-([1,1′- biphenyl]-4- ylcarbamoyl)- 3-acetyl-6- oxodecahydro- pyrrolo[1,2- a][1,5] diazocin-5- yl)amino)-3- oxoprop-1- en-1- yl)phenyl) difluoro- methyl) phosphoryl) bis(oxy))bis (methylene) bis(2,2- dimethylpro- panoate) | 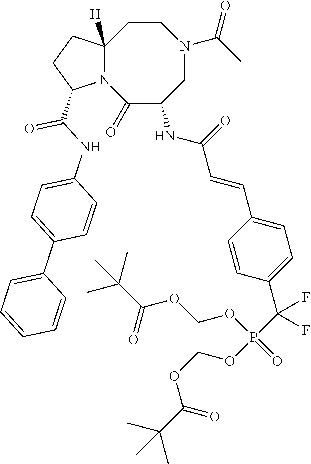 [SMILES: CC(=O)N1CC[C@H]2CC[C@@H](C(=O)Nc3ccc(-c4ccccc4)cc3)N2C(=O)[C@@H](NC(=O)/C=C/c2ccc(C(F)(F)P(=O)(OCOC(=O)C(C)(C)C)OCOC(=O)C(C)(C)C)cc2)C1] | | ¹H NMR (400 MHz, CHLOROFORM-d) δ 8.61 (s, 1H), 7.67-7.56 (m, 10H), 7.44 (t, J = 7.6 Hz, 2H), 7.39-7.32 (m, 1H), 7.12 (d, J = 6.0 Hz, 1H), 6.55 (d, J = 15.4 Hz, 1H), 5.75 (m, 2H), 5.67 (m, 2H), 4.94-4.82 (m, 1H), 4.70 (m, 1H), 4.30- 4.18 (m, 1H), 4.17- 4.03 (m, 2H), 3.12 (m, 1H), 2.95-2.80 (m, 1H), 2.76-2.59 (m, 1H), 2.40 (s, 3H), 2.20 (s, 2H), 2.14 (d, J = 7.6 Hz, 1H), 2.00-1.91 (m, 1H), 1.90-1.81 (m, 1H), 1.23 (s, 18H) |
| 325 | S,S′-(((((4- ((E)-3- (((5S,8S, 10aR)- 8-([1,1′- biphenyl]-4- ylcarbamoyl)- 3-acetyl-6- oxodecahydro- pyrrolo[1,2- a][1,5] diazocin-5- yl)amino)-3- oxoprop-1- en-1- yl)phenyl) difluoro- methyl) phosphoryl) bis(oxy))bis (ethane-2,1- diyl)) bis(3- methylbutane- thioate) | 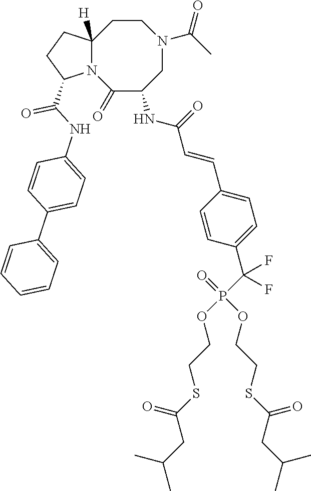 [SMILES: CC(=O)N1CC[C@H]2CC[C@@H](C(=O)Nc3ccc(-c4ccccc4)cc3)N2C(=O)[C@@H](NC(=O)/C=C/c2ccc(C(F)(F)P(=O)(OCCSC(=O)CC(C)C)OCCSC(=O)CC(C)C)cc2)C1] | | ¹H NMR (400 MHz, CHLOROFORM-d) δ 7.65-7.71 (m, 3H) 7.60 (m, 8H) 7.40- 7.46 (m, 2H) 7.31- 7.37 (m, 1H) 7.16 (d, J = 8.0 Hz, 1H) 6.53- 6.62 (m, 1H) 4.84- 4.92 (m, 1H) 4.68 (t, J = 8.0 Hz, 1H) 4.16- 4.30 (m, 5H) 4.04- 4.15 (m, 2H) 3.02- 3.19 (m, 5H) 2.85- 2.97 (m, 1H) 2.55- 2.68 (m, 1H) 2.24- 2.42 (m, 5H) 2.20 (d, J = 4.0 Hz, 4H) 2.02- 2.16 (m, 3H) 1.80- 1.97 (m, 2H) 0.96 (d, J = 8.0 Hz, 12H) |
| 326 | isopropyl (((2- (((5S,8S, 10aR)- 3-acetyl-8- (methyl (phenyl) carbamoyl)-6- oxodecahydro- pyrrolo[1,2- a][1,5] diazocin-5- yl)carbamoyl) benzo[b] thiophen-5- yl)difluoro- methyl) (phenoxy) phosphoryl)- D-alaninate | 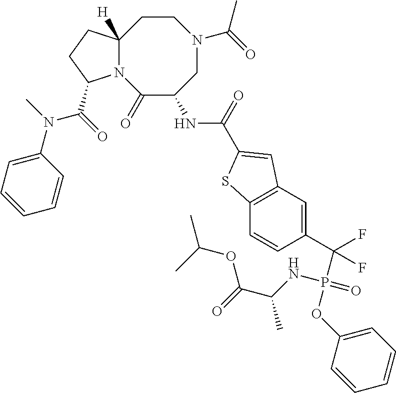 [SMILES: CC(=O)N1CC[C@H]2CC[C@@H](C(=O)N(C)c3ccccc3)N2C(=O)[C@@H](NC(=O)c2cc3cc(C(F)(F)P(=O)(N[C@H](C)C(=O)OC(C)C)Oc4ccccc4)ccc3s2)C1] | 838.2 [M + H]⁺ | ¹H NMR (400 MHz, METHANOL-d₄) δ 8.27-8.13 (m, 2H), 8.12-8.00 (m, 1H), 7.77-7.70 (m, 1H), 7.50-7.29 (m, 7H), 7.23-7.11 (m, 3H), 5.09-5.00 (m, 1H), 4.97-4.87 (m, 2H), 4.46-4.36 (m, 1H), 4.32-4.15 (m, 1H), 4.06-3.98 (m, 1H), 3.93-3.81 (m, 2H), 3.73-3.60 (m, 1H), 3.60-3.47 (m, 1H), 3.28-3.23 (m, 3H), 2.36-2.24 (m, 3H), 2.04-1.98 (m, 2H), 1.97-1.90 (m, 2H), 1.84-1.69 (m, 1H), 1.29-1.26 (m, 3H), 1.21-1.13 (m, 6H) |
| 327 | ((((2- (((5S,8S, 10aR)- 3-acetyl- 8-((4-(2- (dimethyl- amino) ethoxy)phenyl) (methyl) carbamoyl)-6- oxodecahydro- pyrrolo[1,2- a][1,5] diazocin-5- yl)carbamoyl) benzo[b] thiophen-5- yl)difluoro- methyl)phos- phoryl) bis(oxy)) bis(methylene) | 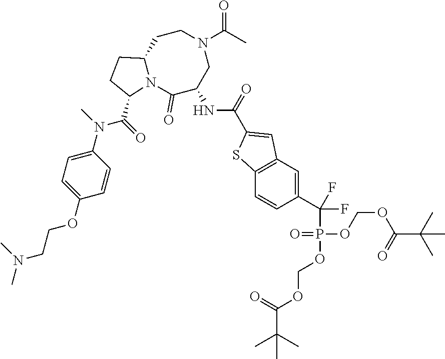 [SMILES: CC(=O)N1CC[C@H]2CC[C@@H](C(=O)N(C)c3ccc(OCCN(C)C)cc3)N2C(=O)[C@@H](NC(=O)c2cc3cc(C(F)(F)P(=O)(OCOC(=O)C(C)(C)C)OCOC(=O)C(C)(C)C)ccc3s2)C1] | 964.0 [M + H]⁺ | |
| | bis(2,2- | | | |
| | dimethylpro- | | | |
| | panoate) | | | |

### Prepared According to General Scheme 4: Phosphonate Esters or Amides

The following compounds in Table 19 were prepared according to the representative procedure described above for the synthesis of ((7-(((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamoyl)naphthalen-2-yl)difluoromethyl)phosphonic acid (4) utilizing the appropriate starting materials and modifications.

TABLE 19

| Compound | Name | Structure | LCMS | NMR |
|---|---|---|---|---|
| 328 | (((difluoro(7-(((5S,8S,10aR)-3-(5-methyl-1H- indazole-3-carbonyl)-8-(methylcarbamoyl)- 6-oxodecahydro-pyrrolo[1,2- a][1,5]diazocin-5-yl)carbamoyl) naphthalen-2-yl)methyl) phosphoryl)bis(oxy))bis(methylene) diisopropylbis(carbonate) | 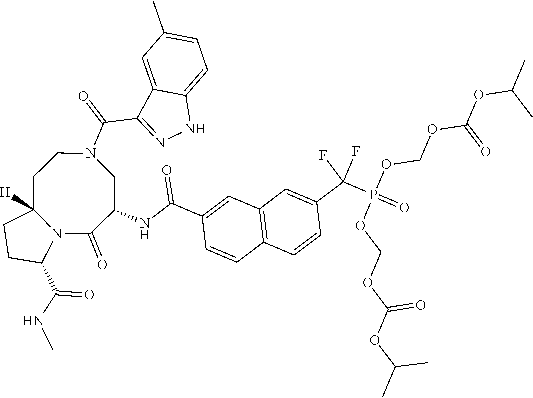 [SMILES: CNC(=O)[C@@H]1CC[C@@H]2CCN(C(=O)c3n[nH]c4ccc(C)cc34)C[C@H](NC(=O)c3ccc4ccc(C(F)(F)P(=O)(OCOC(=O)OC(C)C)OCOC(=O)OC(C)C)cc4c3)C(=O)N21] | 915.1 [M + H]⁺ | ¹H NMR (400 MHZ, METHANOL-d₄) δ 8.61-8.50 (m, 1H), 8.29-8.19 (m, 1H), 8.13-7.47 (m, 5H), 7.45-6.75 (m, 2H), 5.81-5.65 (m, 4H), 5.56-5.20 (m, 1H), 4.82-4.71 (m, 1H), 4.64-4.18 (m, 3H), 4.12-3.55 (m, 3H), 2.64 (s, 3H), 2.46 (s, 3H), 2.38-2.18 (m, 3H), 2.17-1.83 (m, 3H), 1.73-1.55 (m, 1H), 1.32-1.19 (m, 12H). |
| 329 | S,S′-((((difluoro(7-(((5S,8S,10aR)-3-(5-methyl- 1H-indazole-3-carbonyl)-8-(methylcarbamoyl)- 6-oxodecahydropyrrolo[1,2-a][1,5] diazocin-5-yl)carbamoyl) naphthalen-2-yl)methyl)phosphoryl) bis(oxy))bis(ethane-2,1-diyl)) bis(3-methylbutanethioate) | 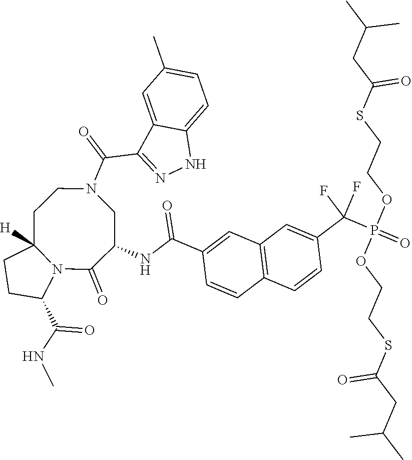 [SMILES: CNC(=O)[C@@H]1CC[C@@H]2CCN(C(=O)c3n[nH]c4ccc(C)cc34)C[C@H](NC(=O)c3ccc4ccc(C(F)(F)P(=O)(OCCSC(=O)CC(C)C)OCCSC(=O)CC(C)C)cc4c3)C(=O)N21] | 971.1 [M + H]⁺ | ¹H NMR (400 MHZ, METHANOL-d₄) δ 8.67-8.54 (m, 1H), 8.34-8.22 (m, 1H), 8.14-7.98 (m, 3H), 7.96-7.84 (m, 1H), 7.76 (d, J = 8.6 Hz, 1H), 7.53-7.21 (m, 2H), 5.55-5.21 (m, 1H), 4.83-4.72 (m, 1H), 4.57-4.16 (m, 7H), 3.95-3.61 (m, 2H), 3.23-3.11 (m, 4H), 2.66 (s, 3H), 2.47 (s, 3H), 2.41 (d, J = 7.1 Hz, 4H), 2.38-2.20 (m, 3H), 2.16-1.84 (m, 5H), 0.90 (d, J = 6.6 Hz, 12H). |
| 330 | S,S′-(((((2-(((5S,8S,10aR)-8-((3S,4R or 3R,4S)- 3-cyano-4-phenylpyrrolidine-1-carbonyl)-3- ethyl-6-oxodecahydropyrrolo[1,2- a][1,5]diazocin-5-yl)carbamoyl) benzo[b]thiophen-5-yl) difluoromethyl)phosphoryl) bis(oxy))bis(ethane-2,1-diyl)) bis(2,2-dimethylpropanethioate) | 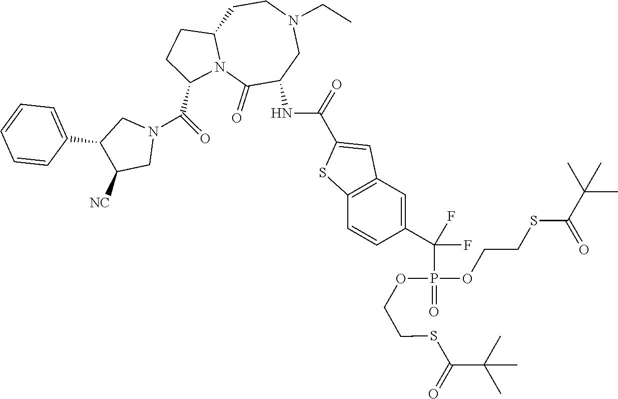 | 988.3 [M + H]⁺ | ¹H NMR (400 MHZ, METHANOL-d₄) δ 8.17 (s, 2H), 8.11-8.08 (m, 1H), 7.67-7.65 (m, 1H), 7.44-7.30 (m, 5H), 5.10-4.97 (m, 1H), 4.77-4.70 (m, 1H), 4.61-4.42 (m, 2H), 4.29-4.12 (m, 4H), 4.12-3.93 (m, 1H), 3.88-3.52 (m, 4H), 3.24-3.05 (m, 6H), 3.05-2.73 (m, 4H), 2.47-2.14 (m, 2H), 2.11-1.97 (m, 1H), 1.96-1.75 (m, 3H), 1.23-1.19 (m, 21H). |
| | | or | | |
| | | 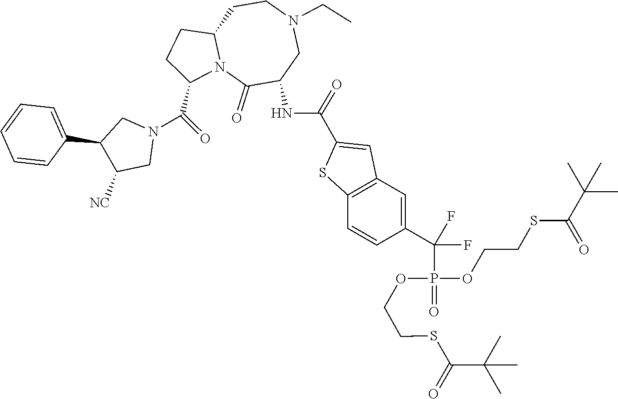 | | |
| | | SFC peak 2 was used for biological testing | | |
| 331 | S,S′-(((((7-(((5S,8S,10aR)-8- (dimethylcarbamoyl)- 3-(5-methyl-1H-indazole-3-carbonyl)-6- oxodecahydropyrrolo[1,2- a][1,5]diazocin-5-yl)carbamoyl) naphthalen-2-yl)difluoromethyl) phosphoryl)bis(oxy))bis(ethane- 2,1-diyl))bis(3-methylbutanethioate) | 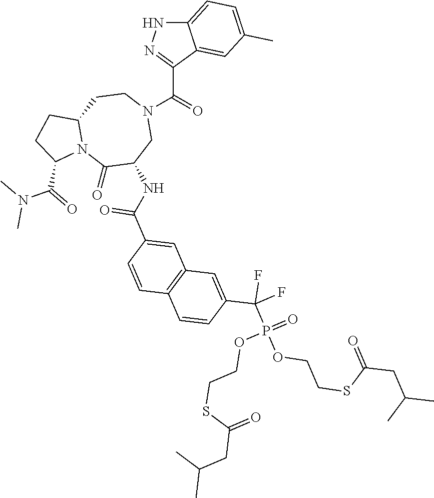 [SMILES: Cc1ccc2[nH]nc(C(=O)N3CC[C@H]4CC[C@@H](C(=O)N(C)C)N4C(=O)[C@@H](NC(=O)c4ccc5ccc(C(F)(F)P(=O)(OCCSC(=O)CC(C)C)OCCSC(=O)CC(C)C)cc5c4)C3)c2c1] | 985.3 [M + H]⁺ | ¹H NMR (400 MHZ, CDCl₃) δ 8.57-8.37 (m, 1H), 8.30-7.69 (m, 7H), 7.12-7.00 (m, 1H), 5.69-5.55 (m, 1H), 4.88 4.80 (m, 1H), 4.57-4.42 (m, 1H), 4.32-4.17 (m, 4H), 3.84-3.70 (m, 1H), 3.26-2.98 (m, 11H), 2.49-2.42 (m, 7H), 2.41-1.99 (m, 8H), 1.95-1.89 (m, 1H), 1.39-1.25 (m, 1H), 0.97-0.92 (m, 12H). |
| 332 | S,S′-(((((7-(((5S,8S,10aR)-3-acetyl- 6-oxo-8-(6-(2-oxo-1,2-dihydropyridin- 4-yl)-4-azaspiro[2.4]heptane-4- carbonyl)decahydropyrrolo[1,2- a][1,5]diazocin-5-yl)carbamoyl) naphthalen-2-yl)difluoromethyl) phosphoryl)bis(oxy))bis(ethane- 2,1-diyl))dibutanethioate | 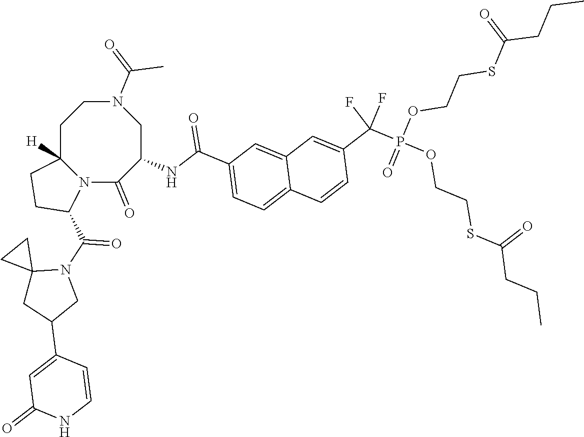 [SMILES: CCCC(=O)SCCOP(=O)(OCCSC(=O)CCC)C(F)(F)c1ccc2ccc(C(=O)N[C@H]3CN(C(C)=O)CC[C@H]4CC[C@@H](C(=O)N5CC(c6cc[nH]c(=O)c6)CC56CC6)N4C3=O)cc2c1] | 986.2 [M + H]⁺ | ¹H NMR (400 MHZ, CDCl₃) δ 8.57-8.43 (m, 1H), 8.41-8.18 (m, 1H), 8.10-8.00 (m, 1H), 7.97 (d, J = 7.7 Hz, 2H), 7.95- 7.83 (m, 1H), 7.79- 7.72 (m, 1H), 7.60- 7.41 (m, 1H), 7.14- 6.65 (m, 1H), 6.64- 6.35 (m, 1H), 4.95- 4.75 (m, 1H), 4.60- 4.41 (m, 1H), 4.31- 3.91 (m, 8H), 3.65- 3.50 (m, 1H), 3.47- 3.22 (m, 2H), 3.20- 3.08 (m, 4H), 2.55- 2.49 (m, 4H), 2.48- 2.44 (m, 3H), 2.41- 2.21 (m, 4H), 2.20- 2.10 (m, 2H), 2.08- 2.00 (m, 2H), 1.96- 1.78 (m, 3H), 1.71- 1.62 (m, 4H), 0.96- 0.90 (m, 6H), 0.62- 0.45 (m, 2H). |
| 333 | isopropyl((benzylamino)((2- (((5S,8S,10aR)-3-(imidazo[1,2- alpyridin-7-ylmethyl)-6-oxo-8-(3- (pyridin-3-yl)azetidine-1-carbonyl) decahydropyrrolo[1,2- a][1,5]diazocin-5-yl)carbamoyl) benzo[b]thiophen-5-yl)methyl) phosphoryl)-L-alaninate | 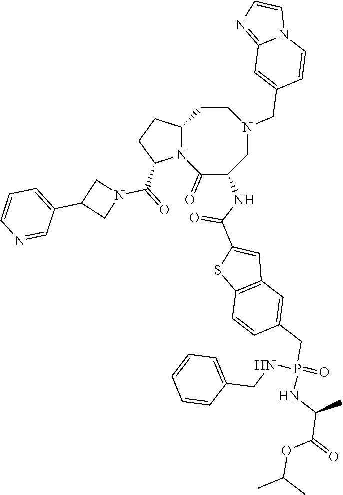 [SMILES: CC(C)OC(=O)[C@H](C)NP(=O)(Cc1ccc2sc(C(=O)N[C@H]3CN(Cc4ccn5ccnc5c4)CC[C@H]4CC[C@@H](C(=O)N5CC(c6cccnc6)C5)N4C3=O)cc2c1)NCc1ccccc1] | 930.0 [M + H]⁺ | ¹H NMR (400 MHZ, CDCl₃) δ 9.01-8.78 (m, 1H), 8.75-8.45 (m, 2H), 8.36-8.24 (m, 1H), 8.22-8.12 (m, 1H), 8.10-8.02 (m, 1H), 7.98-7.90 (m, 1H), 7.89-7.60 (m, 7H), 7.40-7.30 (m, 3H), 7.30-7.29 (m, 1H), 7.23-7.20 (m, 1H), 5.20-5.09 (m, 1H), 5.02-4.74 (m, 2H), 4.70-4.59 (m, 1H), 4.57-4.35 (m, 3H), 4.27-4.18 (m, 1H), 4.13-4.02 (m, 4H), 4.01-3.92 (m, 2H), 3.30-3.22 (m, 2H), 3.09-2.93 (m, 4H), 2.24-2.19 (m, 2H), 1.95-1.88 (m, 4H), 1.32 (d, J = 6.5 Hz, 3H), 1.21- 1.16 (m, 6H). |
| 334 | S-(2-((((7-(((5S,8S,10aR)-2-(2,2- difluoroethyl)-6-oxo-8-(6- (2-oxo-1,2-dihydropyridin-4- yl)-4-azaspiro[2.4]heptane-4- carbonyl)decahydropyrrolo[1,2- a][1,4]diazocin-5-yl)carbamoyl) naphthalen-2-yl)difluoromethyl) (((isopropoxycarbonyl)oxy)methoxy) phosphoryl)oxy)ethyl)butanethioate | 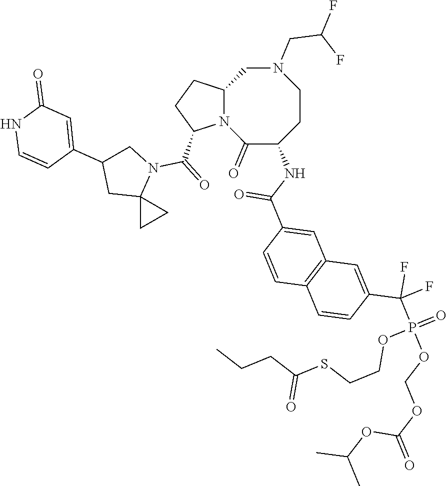 [SMILES: CCCC(=O)SCCOP(=O)(OCOC(=O)OC(C)C)C(F)(F)c1ccc2ccc(C(=O)N[C@H]3CCN(CC(F)F)C[C@H]4CC[C@@H](C(=O)N5CC(c6cc[nH]c(=O)c6)CC56CC6)N4C3=O)cc2c1] | 994.0 [M + H]⁺ | ¹H NMR (400 MHZ, CDCl₃) δ 8.48-8.35 (m, 1H), 8.28-8.15 (m, 1H), 8.09-7.30 (m, 7H), 7.22-6.60 (m, 3H), 6.32-6.08 (m, 1H), 5.75-5.60 (m, 4H), 5.51-5.41 (m, 1H), 4.98-4.61 (m, 3H), 4.30-3.95 (m, 3H), 3.85-3.23 (m, 6H), 3.18-3.07 (m, 3H), 2.53-2.47 (m, 2H), 2.31-2.20 (m, 2H), 2.15-2.09 (m, 1H), 1.97-1.80 (m, 3H), 1.72-1.54 (m, 3H), 1.39-1.30 (m, 6H), 0.98-0.88 (m, 4H), 0.81-0.42 (m, 2H). |
| 335 | S,S′-(((((7-(((5S,8S,10aR)-2-(2,2- difluoroethyl)-6-oxo-8-(6-(2-oxo-1,2- dihydropyridin-4-yl)-4-azaspiro[2.4] heptane-4-carbonyl)decahydropyrrolo [1,2-a][1,4]diazocin-5-yl)carbamoyl) naphthalen-2-yl)difluoromethyl) phosphoryl)bis(oxy))bis(ethane- 2,1-diyl))dibutanethioate | 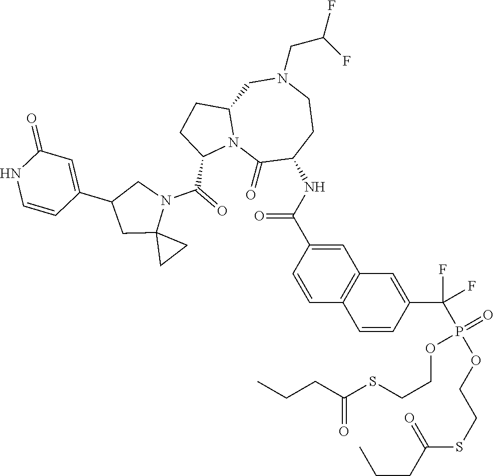 [SMILES: CCCC(=O)SCCOP(=O)(OCCSC(=O)CCC)C(F)(F)c1ccc2ccc(C(=O)N[C@H]3CCN(CC(F)F)C[C@H]4CC[C@@H](C(=O)N5CC(c6cc[nH]c(=O)c6)CC56CC6)N4C3=O)cc2c1] | 505.1 [M/2 + H] | ¹H NMR (400 MHZ, DMSO-d6) δ 8.82- 8.63 (m, 2H), 8.27 (s, 1H), 8.18-8.04 (m, 3H), 7.71 (d, J = 8.6 Hz, 1H), 7.35-7.18 (m, 1H), 6.28-6.16 (m, 2H), 5.24-5.13 (m, 1H), 4.71-4.50 (m, 3H), 4.31-4.02 (m, 10H), 3.39-3.00 (m, 9H), 2.57-2.52 (m, 3H), 2.11-1.90 (m, 6H), 1.74-1.65 (m, 2H), 1.60-1.45 (m, 6H), 0.86-0.81 (m, 6H), 0.53-0.42 (m, 2H). |
| 336 | S,S′-(((((7-(((5S,8S,10aR)-8-((rel-trans)-3- cyano-4-phenylpyrrolidine-1-carbonyl)-3- (5-methyl-1H-indazole-3-carbonyl)-6- oxodecahydropyrrolo[1,2-a][1,5]diazocin- 5-yl)carbamoyl)naphthalen- 2-yl)difluoromethyl) phosphoryl)bis(oxy))bis(ethane-2,1-diyl)) dibutanethioate | 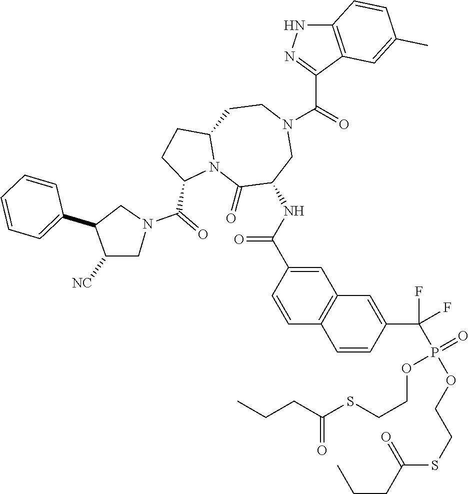 | 1084.2 [M + H]⁺ | ¹H NMR (400 MHZ, CDCl₃) δ 8.51-8.37 (m, 1H), 8.16-8.10 (m, 1H), 8.08-7.99 (m, 1H), 7.97-7.80 (m, 3H), 7.77-7.71 (m, 1H), 7.45-7.27 (m, 6H), 7.26-7.20 (m, 1H), 7.15-7.04 (m, 1H), 5.73-5.54 (m, 1H), 4.71-3.44 (m, 17H), 3.35-3.19 (m, 1H), 3.19-3.08 (m, 4H), 2.54 2.46 (m, 5H), 2.41-2.30 (m, 2H), 2.30-2.13 (m, 3H), 2.00-1.89 (m, 1H), 1.71-1.60 (m, 4H), 0.98-0.86 (m, 6H). |
| 337 | isopropyl(((2-(((5S,8S,10aR)-8- (dimethylcarbamoyl)-3-(5-methyl-1H-indazole- 3-carbonyl)-6-oxodecahydropyrrolo[1,2- a][1,5]diazocin-5-yl)carbamoyl)benzo [b]thiophen-5-yl)methyl)(phenoxy)phosphoryl)- L-alaninate | 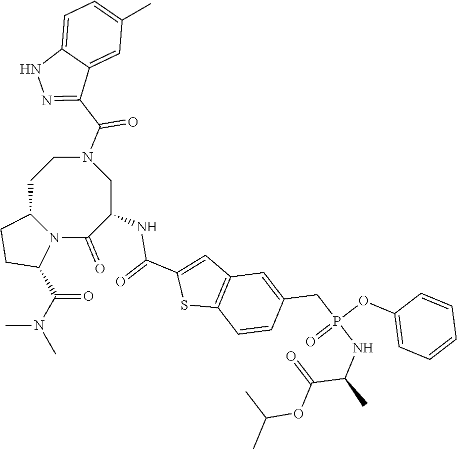 [SMILES: Cc1ccc2[nH]nc(C(=O)N3CC[C@H]4CC[C@@H](C(=O)N(C)C)N4C(=O)[C@@H](NC(=O)c4cc5cc(CP(=O)(N[C@@H](C)C(=O)OC(C)C)Oc6ccccc6)ccc5s4)C3)c2c1] | 856.4 [M + H]⁺ | ¹H NMR (400 MHZ, DMSO) δ 13.53- 13.36 (m, 1H), 9.05- 8.83 (m, 1H), 8.25- 8.12 (m, 1H), 8.00- 7.82 (m, 3H), 7.53- 7.39 (m, 2H), 7.36- 7.28 (m, 2H), 7.27- 7.20 (m, 1H), 7.18- 7.08 (m, 3H), 5.70- 5.50 (m, 1H), 5.19- 4.90 (m, 1H), 4.86- 4.71 (m, 3H), 4.55- 4.14 (m, 2H), 3.87- 3.70 (m, 1H), 3.65- 3.56 (m, 1H), 3.51- 3.39 (m, 2H), 3.05 (s, 3H), 2.81 (s, 3H), 2.43-2.39 (m, 3H), 2.37- 2.23 (m, 1H), 2.18- 1.92 (m, 2H), 1.89- 1.74 (m, 2H), 1.73- 1.63 (m, 1H), 1.14- 1.07 (m, 6H), 1.07- 1.02 (m, 3H). |
| 338 | ((((2-(((5S,8S,10aR)-3-(imidazo[1,2-a]pyridin-7- ylmethyl)-6-oxo-8-(3-(pyridin-3-yl)azetidine- 1-carbonyl)decahydropyrrolo[1,2- a][1,5]diazocin-5-yl)carbamoyl)benzo[b] thiophen-5-yl)methyl)phosphoryl)bis (oxy))bis(methylene)diisopropylbis(carbonate) | 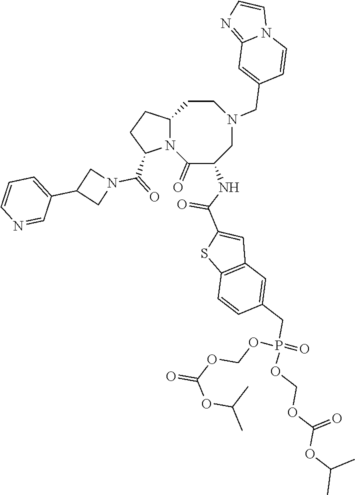 [SMILES: CC(C)OC(=O)OCOP(=O)(Cc1ccc2sc(C(=O)N[C@H]3CN(Cc4ccn5ccnc5c4)CC[C@H]4CC[C@@H](C(=O)N5CC(c6cccnc6)C5)N4C3=O)cc2c1)OCOC(=O)OC(C)C] | 960.4 [M + H]⁺ | ¹H NMR (400 MHZ, CDCl₃) δ 8.85-8.59 (m, 1H), 8.58-8.50 (m, 1H), 8.36-8.21 (m, 1H), 8.16-8.04 (m, 1H), 7.95-7.81 (m, 1H), 7.81-7.55 (m, 6H), 7.41-7.29 (m, 2H), 7.23-7.15 (m, 1H), 5.65-5.55 (m, 4H), 5.19-5.05 (m, 1H), 4.95-4.85 (m, 2H), 4.77-4.62 (m, 1H), 4.52-4.39 (m, 2H), 4.28-4.17 (m, 1H), 4.14-3.88 (m, 3H), 3.44-3.32 (m, 2H), 3.30-3.04 (m, 2H), 3.03-2.90 (m, 2H), 2.26-2.19 (m, 2H), 2.06-1.97 (m, 2H), 1.64-1.55 (m, 2H), 1.54-1.40 (m, 2H), 1.30 (d, J = 6.2 Hz, 12H). |
| 339 | S,S′-(((((R or S)-(2-(((5S,8S,10aR)-8-((3S,4R or 3R,4S)-3-cyano-4-phenylpyrrolidine-1- carbonyl)-3-ethyl-6-oxodecahydropyrrolo[1,2- a][1,5]diazocin-5-yl)carbamoyl)benzo[b] thiophen-5-yl)fluoromethyl)phosphoryl)bis(oxy)) bis(ethane-2,1-diyl))bis(3-methylbutane thioate) | 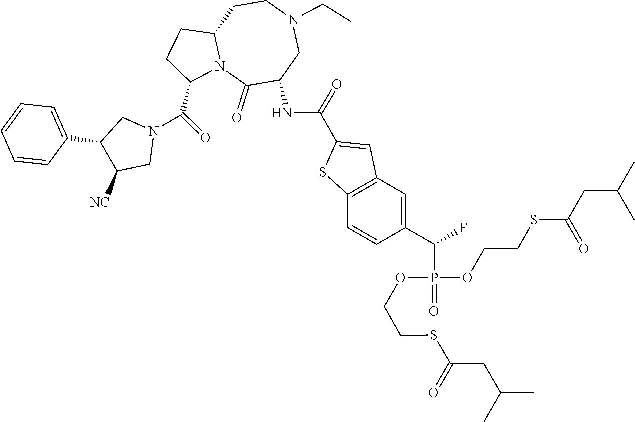 | 970 [M + H]⁺ | ¹H NMR (400 MHZ, CDCl₃) δ 7.95 (s, 1H), 7.92-7.77 (m, 2H), 7.58-7.29 (m, 7H), 5.83 (dd, J = 44.4, 7.4 Hz, 1H), 5.14-4.93 (m, 1H), 4.67-4.49 (m, 2H), 4.36-4.23 (m, 1H), 4.20-3.94 (m, 6H), 3.83-3.64 (m, 2H), 3.55-3.17 (m, 3H), 3.16-2.99 (m, 6H), 2.94-2.85 (m, 2H), 2.46-2.38 (m, 4H), 2.35-2.26 (m, 1H), 2.19-2.08 (m, 4H), 2.02-1.71 (m, 5H), 1.36-1.22 (m, 1H), 0.94 (d, J = 6.7 Hz, 12H). |
| | | or | | |
| | | 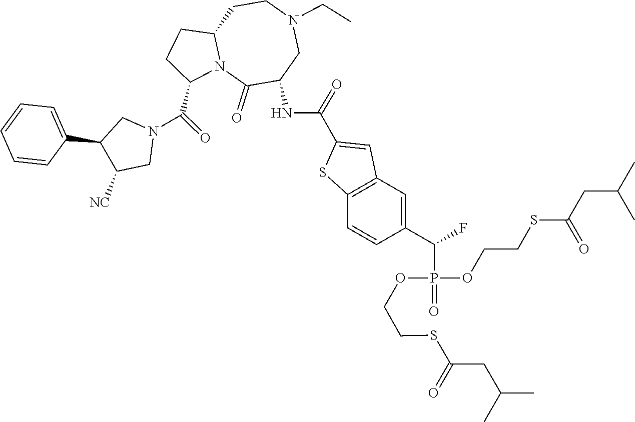 | | |
| | | or | | |
| | | 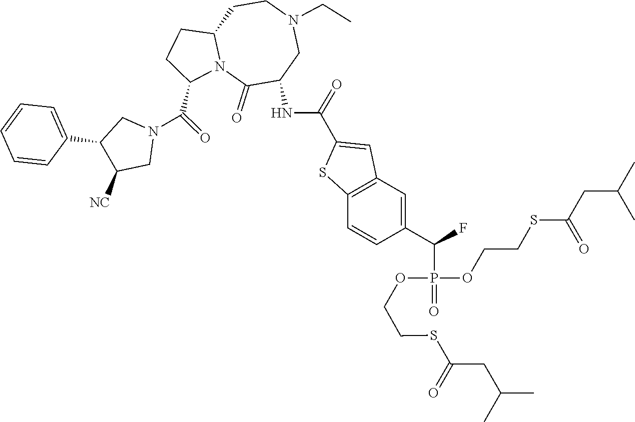 | | |
| | | or | | |
| | | 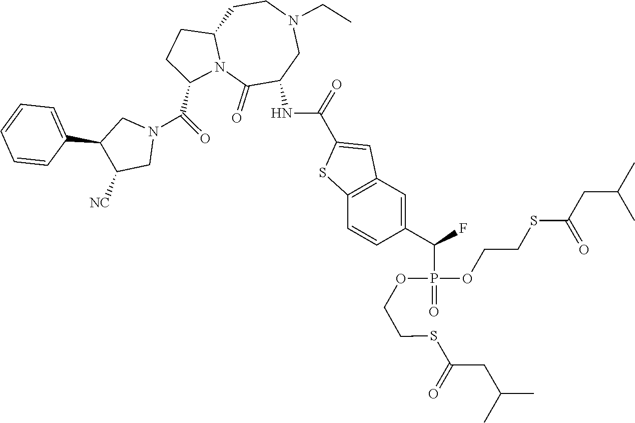 | | |
| | | SFC peak 2 was used for biological testing, Benzothiophene peak 1 was used for synthesis | | |
| 340 | S,S′-(((((7-(((5S,8S,10aR)-8-((rel-trans)-3- cyano-4-phenylpiperidine-1-carbonyl)-3- (2,2-difluoroethyl)-6-oxodecahydropyrrolo[1,2- a][1,5]diazocin-5-yl)carbamoyl)naphthalen- 2-yl)difluoromethyl)phosphoryl)bis(oxy))bis (ethane-2,1-diyl))bis(2,2-dimethylpropanethioate) | 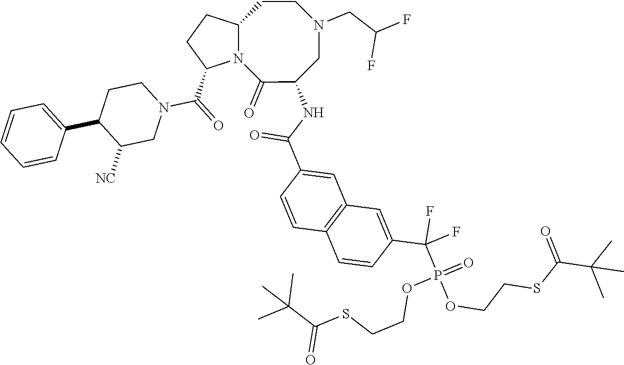 [SMILES: CC(C)(C)C(=O)SCCOP(=O)(OCCSC(=O)C(C)(C)C)C(F)(F)c1ccc2ccc(C(=O)N[C@H]3CN(CC(F)F)CC[C@H]4CC[C@@H](C(=O)N5CC[C@H](c6ccccc6)[C@@H](C#N)C5)N4C3=O)cc2c1] | 1032 [M + H]⁺ | ¹H NMR (400 MHZ, DMSO) δ 8.79-8.59 (m, 1H), 8.55-8.25 (m, 2H), 8.22-7.97 (m, 3H), 7.79-7.67 (m, 1H), 7.47-7.15 (m, 5H), 6.35-5.90 (m, 1H), 5.39-4.04 (m, 10H), 3.25-2.81 (m, 13H), 2.14-1.90 (m, 2H), 1.88-1.30 (m, 6H), 1.19-1.05 (m, 18H). |
| 341 | S,S′-(((((7-(((5S,8S,10aR)-8-((rel-trans)-3- cyano-4-(3,5-difluorophenyl)pyrrolidine-1- carbonyl)-3-(2,2-difluoroethyl)-6- oxodecahydropyrrolo[1,2-a][1,5]diazocin-5- yl)carbamoyl)naphthalen-2-yl) difluoromethyl)phosphoryl)bis(oxy))bis(ethane- 2,1-diyl))bis(3-methylbutanethioate) | 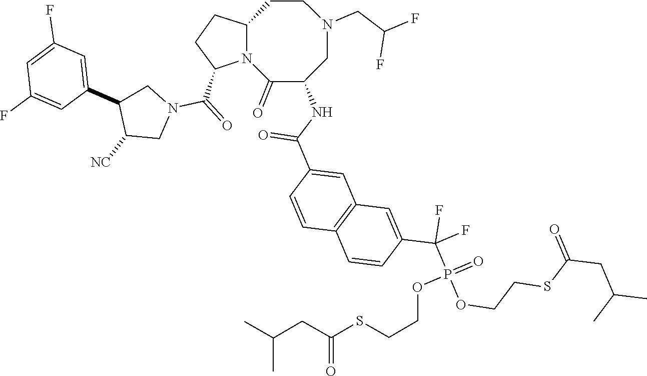 [SMILES: CC(C)CC(=O)SCCOP(=O)(OCCSC(=O)CC(C)C)C(F)(F)c1ccc2ccc(C(=O)N[C@H]3CN(CC(F)F)CC[C@H]4CC[C@@H](C(=O)N5C[C@H](c6cc(F)cc(F)c6)[C@@H](C#N)C5)N4C3=O)cc2c1] | 1054 [M + H]⁺ | ¹H NMR (400 MHZ, CDCl₃) δ 8.55-8.34 (m, 1H), 8.22 (s, 1H), 8.09-7.89 (m, 3H), 7.77 (d, J = 8.0 Hz, 1H), 7.73-7.46 (m, 1H), 7.14-6.75 (m, 3H), 6.13 (t, J = 55.8 Hz, 1H), 5.18-4.99 (m, 1H), 4.68-4.51 (m, 2H), 4.37-3.96 (m, 6H), 3.94-3.37 (m, 4H), 3.36-2.97 (m, 10H), 2.41 (d, J = 7.0 Hz, 4H), 2.35-2.25 (m, 1H), 2.23-2.07 (m, 4H), 1.93-1.86 (m, 1H), 1.56-1.51 (m, 1H), 1.45 (d, J = 6.7 Hz, 1H), 0.93 (d, J = 6.6 Hz, 12H). |
| 342 | S,S′-(((((7-(((5S,8S,10aR)-8-((3S,4R or 3R,4S)-3-cyano-4-phenylpyrrolidine-1- carbonyl)-3-(2,2-difluoroethyl)-6- oxodecahydropyrrolo[1,2-a][1,5]diazocin-5- yl)carbamoyl)naphthalen-2-yl) difluoromethyl)phosphoryl)bis(oxy))bis (ethane-2,1-diyl))bis(2,2-dimethylprop anethioate) | 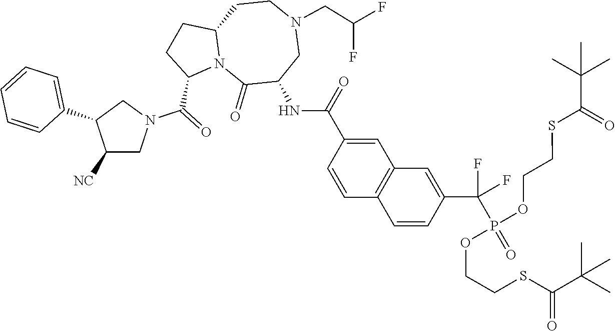 [SMILES: CC(C)(C)C(=O)SCCOP(=O)(OCCSC(=O)C(C)(C)C)C(F)(F)c1ccc2ccc(C(=O)N[C@H]3CN(CC(F)F)CC[C@H]4CC[C@@H](C(=O)N5C[C@@H](C#N)[C@H](c6ccccc6)C5)N4C3=O)cc2c1] | 1018 [M + H]⁺ | ¹H NMR (400 MHZ, DMSO) δ 8.76-8.62 (m, 1H), 8.49-8.26 (m, 2H), 8.20-8.00 (m, 3H), 7.79-7.66 (m, 1H), 7.53-7.27 (m, 5H), 6.30-5.94 (m, 1H), 5.05-4.87 (m, 1H), 4.65-4.49 (m, 1H), 4.43-4.30 (m, 1H), 4.27-3.46 (m, 10H), 3.24-2.93 (m, 10H), 2.39-2.26 (m, 1H), 2.17-1.98 (m, 1H), 1.95-1.61 (m, 4H), 1.14 (s, 18H). |
| | | or | | |
| | | 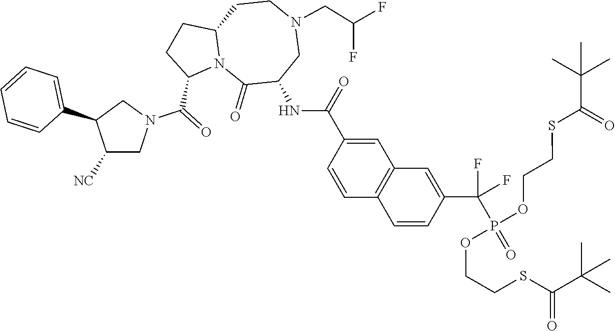 [SMILES: CC(C)(C)C(=O)SCCOP(=O)(OCCSC(=O)C(C)(C)C)C(F)(F)c1ccc2ccc(C(=O)N[C@H]3CN(CC(F)F)CC[C@H]4CC[C@@H](C(=O)N5C[C@H](c6ccccc6)[C@@H](C#N)C5)N4C3=O)cc2c1] | | |
| 343 | S,S′-(((((7-(((5S,8S,10aR)-8-((3S,4R or 3R,4S)-3-cyano-4-phenylpyrrolidine-1- carbonyl)-3-ethyl-6-oxodecahydropyrrolo[1,2- a][1,5]diazocin-5-yl)carbamoyl)naphthalen- 2-yl)difluoromethyl)phosphoryl)bis(oxy)) bis(ethane-2,1-diyl))bis(2,2- dimethylpropanethioate) | 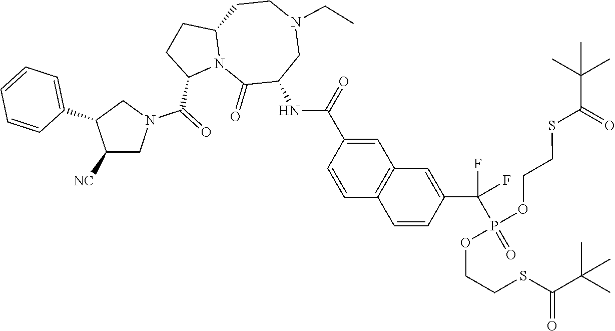 [SMILES: CCN1CC[C@H]2CC[C@@H](C(=O)N3C[C@@H](C#N)[C@H](c4ccccc4)C3)N2C(=O)[C@@H](NC(=O)c2ccc3ccc(C(F)(F)P(=O)(OCCSC(=O)C(C)(C)C)OCCSC(=O)C(C)(C)C)cc3c2)C1] | 982 [M + H]⁺ | ¹H NMR (400 MHZ, DMSO-d₆) δ 9.75- 8.94 (m, 1H), 8.67 (s, 1H), 8.30 (s, 1H), 8.21- 8.13 (m, 2H), 8.10- 8.02 (m, 1H), 7.81- 7.69 (m, 1H), 7.48- 7.29 (m, 5H), 5.62- 5.49 (m, 1H), 4.96- 4.66 (m, 2H), 4.27- 4.06 (m, 6H), 4.04- 3.78 (m, 5H), 3.78- 3.63 (m, 5H), 3.18- 3.04 (m, 4H), 2.21- 2.04 (m, 2H), 1.98- 1.83 (m, 3H), 1.41- 1.28 (m, 4H), 1.13 (s, 18H). (TFA salt). |
| | | or | | |
| | | 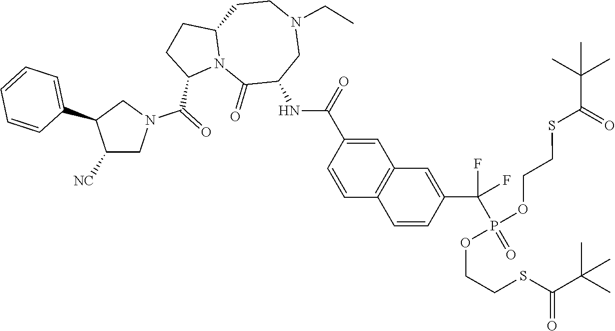 [SMILES: CCN1CC[C@H]2CC[C@@H](C(=O)N3C[C@H](c4ccccc4)[C@@H](C#N)C3)N2C(=O)[C@@H](NC(=O)c2ccc3ccc(C(F)(F)P(=O)(OCCSC(=O)C(C)(C)C)OCCSC(=O)C(C)(C)C)cc3c2)C1] | | |
| | | SFC peak 2 was used for biolical testing | | |
| 344 | ((((7-(((5S,8S,10aR)-8-((rel-trans)-3- cyano-4-(3,5-difluorophenyl)pyrrolidine-1- carbonyl)-3-(2,2-difluoroethyl)-6- oxodecahydropyrrolo[1,2-a][1,5]diazocin-5- yl)carbamoyl)naphthalen-2-yl) difluoromethyl)phosphoryl)bis(oxy)) bis(methylene) bis(2,2-dimethylpropanoate) | 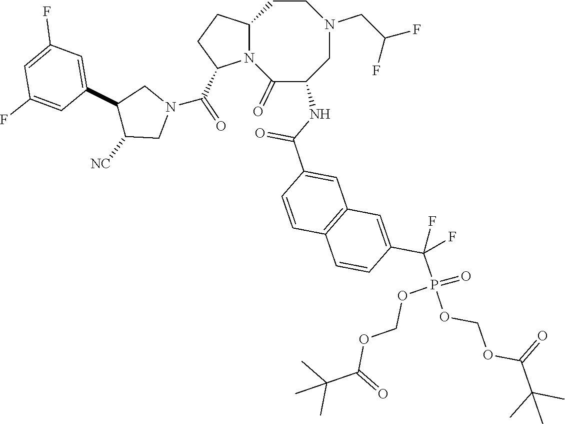 | 994 [M + H]⁺ | ¹H NMR (400 MHZ, CDCl₃) δ 8.45-8.35 (m, 1H), 8.28-8.16 (m, 1H), 8.07-7.90 (m, 3H), 7.78-7.72 (m, 1H), 7.71-7.56 (m, 1H), 7.15-6.78 (m, 3H), 6.04 (t, J = 55.6 Hz, 1H), 5.80- 5.61 (m, 4H), 5.08- 4.94 (m, 1H), 4.72- 3.98 (m, 5H), 3.91- 3.64 (m, 2H), 3.56- 3.31 (m, 2H), 3.30- 3.13 (m, 4H), 3.09- 2.94 (m, 1H), 2.33- 2.12 (m, 3H), 2.00- 1.85 (m, 3H), 1.22 (s, 18H). |
| 345 | ((((7-(((5S,8S,10aR)-8-((rel-trans)-3- cyano-4-phenylpiperidine-1-carbonyl)-3- (2,2-difluoroethyl)-6-oxodecahydropyrrolo[1,2- a][1,5]diazocin-5-yl)carbamoyl)naphthalen- 2-yl)difluoromethyl)phosphoryl)bis(oxy)) bis(methylene) bis(2,2-dimethylpropanoate) | 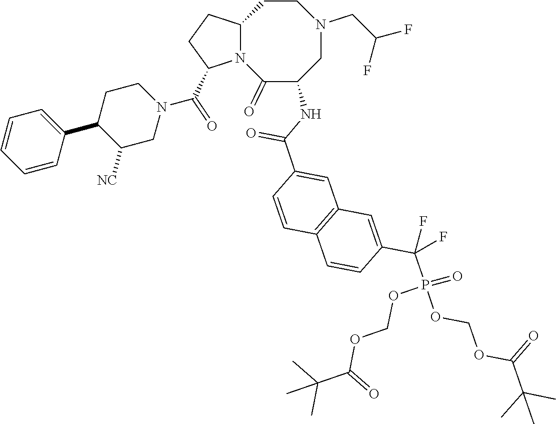 | 972 [M + H]⁺ | ¹H NMR (400 MHZ, CDCl₃) δ 8.51-8.36 (m, 1H), 8.28-8.13 (m, 1H), 8.03-7.89 (m, 3H), 7.82-7.60 (m, 2H), 7.46-7.28 (m, 5H), 6.23-5.82 (m, 1H), 5.78-5.62 (m, 4H), 5.03-4.81 (m, 2H), 4.66-4.03 (m, 2H), 3.51-2.64 (m, 11H), 2.37-1.88 (m, 8H), 1.20 (s, 18H). |
| 346 | ((((7-(((5S,8S,10aR)-8-((3S,4Ror 3R,4S)-3- cyano-4-phenylpyrrolidine-1-carbonyl)-3- (2,2-difluoroethyl)-6-oxodecahydropyrrolo[1,2- a][1,5]diazocin-5-yl)carbamoyl)naphthalen- 2-yl)difluoromethyl)phosphoryl)bis(oxy)) bis(methylene) bis(2,2-dimethylpropanoate) | 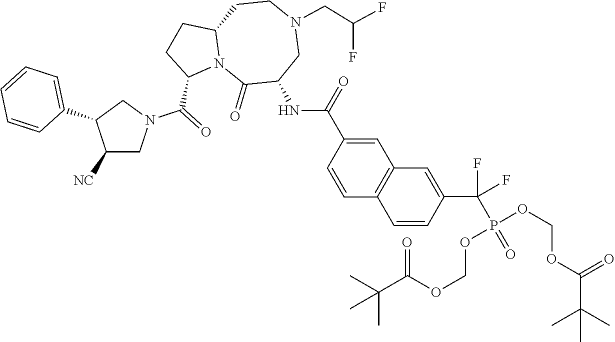 [SMILES: CC(C)(C)C(=O)OCOP(=O)(OCOC(=O)C(C)(C)C)C(F)(F)c1ccc2ccc(C(=O)N[C@H]3CN(CC(F)F)CC[C@H]4CC[C@@H](C(=O)N5C[C@@H](C#N)[C@H](c6ccccc6)C5)N4C3=O)cc2c1] | 958 [M + H]⁺ | ¹H NMR (400 MHZ, CDCl₃) δ 8.46-8.34 (m, 1H), 8.25-8.16 (m, 1H), 8.03-7.90 (m, 3H), 7.79-7.71 (m, 1H), 7.67-7.59 (m, 1H), 7.47-7.29 (m, 5H), 6.15-5.80 (m, 1H), 5.79-5.61 (m, 4H), 5.03-4.90 (m, 1H), 4.64-4.42 (m, 2H), 4.37-4.00 (m, 3H), 3.83-3.61 (m, 2H), 3.57-3.27 (m, 2H), 3.26-3.13 (m, 4H), 3.09-2.97 (m, 1H), 2.38-2.26 (m, 1H), 2.21-2.08 (m, 2H), 1.97-1.81 (m, 3H), 1.20 (s, 18H). |
| 347 | S,S′-(((((7-(((5S,8S,10aR)-3-(2,2- difluoroethyl)-8-(methyl(1-(methylsulfonyl) piperidin-4-yl)carbamoyl)-6-oxodeca hydropyrrolo[1,2-a][1,5]diazocin-5-yl)carbamoyl) naphthalen-2-yl)difluoromethyl)phosphoryl) bis(oxy))bis(ethane-2,1-diyl))dibutanethioate | 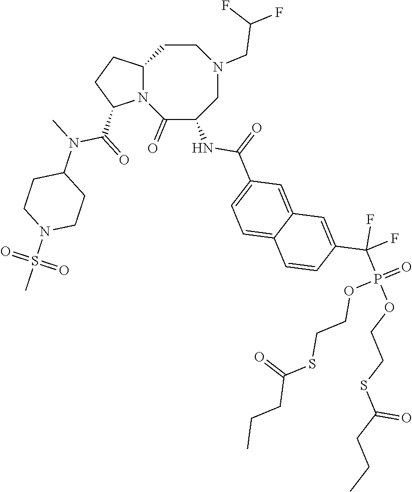 [SMILES: CCCC(=O)SCCOP(=O)(OCCSC(=O)CCC)C(F)(F)c1ccc2ccc(C(=O)N[C@H]3CN(CC(F)F)CC[C@H]4CC[C@@H](C(=O)N(C)C5CCN(S(C)(=O)=O)CC5)N4C3=O)cc2c1] | 1010.3 [M + H]⁺ | ¹H NMR (400 MHZ, CDCl₃) δ 8.41 (s, 1H), 8.19 (s, 1H), 8.07- 7.84 (m, 3H), 7.81- 7.63 (m, 2H), 6.13- 5.80 (m, 1H), 4.99- 4.89 (m, 1H), 4.78 (t, J = 8.6 Hz, 1H), 4.70- 4.56 (m, 1H), 4.54- 4.42 (m, 1H), 4.32- 4.12 (m, 4H), 4.01- 3.86 (m, 2H), 3.40- 3.25 (m, 2H), 3.18- 3.11 (m, 4H), 3.02 (s, 3H), 2.83-2.73 (m, 4H), 2.52 (t, J = 7.4 Hz, 4H), 2.40-2.30 (m, 1H), 2.26-2.08 (m, 2H), 2.08-1.95 (m, 2H), 1.94-1.79 (m, 5H), 1.79-1.70 (m, 5H), 1.70-1.64 (m, 4H), 0.94 (t, J = 7.4 Hz, 6H). |
| 348 | ((((2-(((5S,8S,10aR)-8-((3S,4R or 3R,4S)-3- cyano-4-phenylpyrrolidine-1-carbonyl)-3- (2,2-difluoroethyl)-6-oxodecahydropyrrolo[1,2- a][1,5]diazocin-5-yl)carbamoyl)benzo[b] thiophen-5-yl)difluoromethyl)phosphoryl)bis (oxy)) bis(methylene) bis(2,2- dimethylpropanoate) | 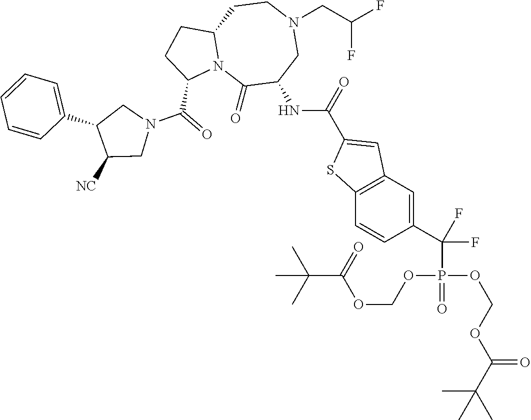 [SMILES: CC(C)(C)C(=O)OCOP(=O)(OCOC(=O)C(C)(C)C)C(F)(F)c1ccc2sc(C(=O)N[C@H]3CN(CC(F)F)CC[C@H]4CC[C@@H](C(=O)N5C[C@@H](C#N)[C@H](c6ccccc6)C5)N4C3=O)cc2c1] | 964 [M + H]⁺ | ¹H NMR (400 MHZ, CDCl₃) δ 8.05-7.98 (m, 1H), 7.87 (d, J = 8.6 Hz, 1H), 7.82- 7.73 (m, 1H), 7.57 (d, J = 8.5 Hz, 1H), 7.49- 7.40 (m, 1H), 7.39- 7.23 (m, 5H), 6.01- 5.79 (m, 1H), 5.70- 5.52 (m, 4H), 4.87- 4.73 (m, 1H), 4.58- 4.47 (m, 1H), 4.41- 4.32 (m, 1H), 4.28- 3.92 (m, 3H), 3.76- 3.57 (m, 2H), 3.31- 2.91 (m, 7H), 2.11- 2.01 (m, 2H), 1.98- 1.73 (m, 4H), 1.13 (s, 18H). |
| | | or | | |
| | | 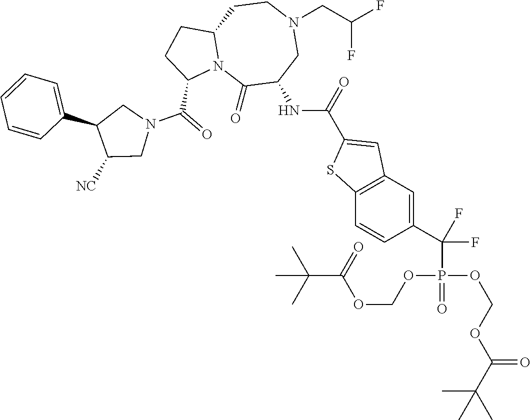 [SMILES: CC(C)(C)C(=O)OCOP(=O)(OCOC(=O)C(C)(C)C)C(F)(F)c1ccc2sc(C(=O)N[C@H]3CN(CC(F)F)CC[C@H]4CC[C@@H](C(=O)N5C[C@H](c6ccccc6)[C@@H](C#N)C5)N4C3=O)cc2c1] | | |
| | | SFC peak 2 was used for bilogical testing | | |
| 349 | ((((2-(((5S,8S,10aR)-8-((rel-trans)-3- cyano-4-phenylpyrrolidine-1- carbonyl)-3-(methoxycarbonyl)-6- oxodecahydropyrrolo[1,2-a][1,5]diazocin-5- yl)carbamoyl)benzo[b]thiophen-5- yl)difluoromethyl)phosphoryl)bis(oxy)) bis(methylene) bis(2,2-dimethylpropanoate) | 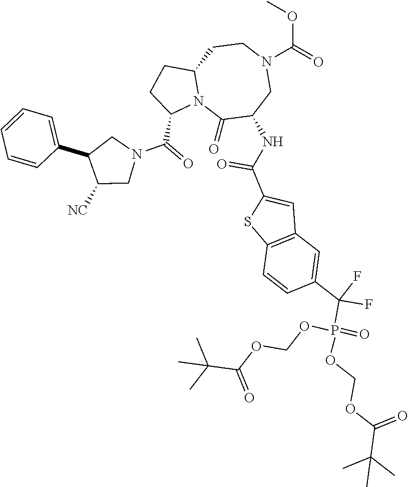 [SMILES: COC(=O)N1CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(OCOC(=O)C(C)(C)C)OCOC(=O)C(C)(C)C)ccc3s2)C1] | 958 [M + H]⁺ | ¹H NMR (400 MHZ, CDCl₃) δ 8.16-8.06 (m, 1H), 8.00-7.87 (m, 2H), 7.68-7.28 (m, 6H), 5.78-5.70 (m, 2H), 5.70-5.62 (m, 2H), 5.11-4.99 (m, 1H), 4.68-4.17 (m, 4H), 4.09-3.96 (m, 1H), 3.97-3.63 (m, 6H), 3.61-3.48 (m, 1H), 3.45-3.00 (m, 2H), 2.40-2.29 (m, 1H), 2.25-2.05 (m, 3H), 2.03-1.84 (m, 2H), 1.28-1.26 (m, 1H), 1.26-1.16 (m, 18H). |
| 350 | ((((2-(((5S,8S,10aR)-3-(2,2-difluoroethyl)- 8-(3-(morpholine-4-carbonyl)azetidine-1- carbonyl)-6-oxodecahydropyrrolo[1,2- a][1,5]diazocin-5-yl)carbamoyl)benzo[b] thiophen-5-yl)difluoromethyl)phosphoryl)bis (oxy))bis(methylene) bis(2,2- dimethylpropanoate) | 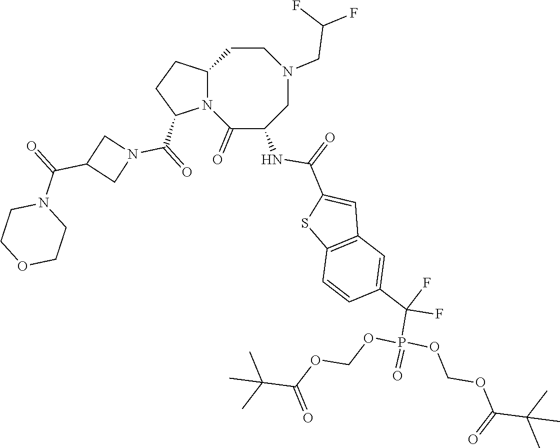 [SMILES: CC(C)(C)C(=O)OCOP(=O)(OCOC(=O)C(C)(C)C)C(F)(F)c1ccc2sc(C(=O)N[C@H]3CN(CC(F)F)CC[C@H]4CC[C@@H](C(=O)N5CC(C(=O)N6CCOCC6)C5)N4C3=O)cc2c1] | 962.2 [M + H]⁺ | ¹H NMR (400 MHZ, CDCl₃) δ 8.05 (m, 1H), 7.89 (m, 1H), 7.82 (m, 1H), 7.60 (m, 2H), 6.02 (m, 1H), 5.71 (m, 2H), 5.67-5.57 (m, 2H), 4.96 (m, 1H), 4.75- 4.53 (m, 2H), 4.49 (s, 1H), 4.39 (m, 2H), 4.27 (s, 1H), 4.09 (m, 1H), 3.67 (m, 7H), 3.47 (s, 1H), 3.27 (m, 6H), 3.06 (m, 1H), 2.26 (s, 1H), 2.08 (s, 2H), 1.85 (s, 2H), 1.18 (s, 18H). |
| 351 | ((((2-(((5S,8S,10aR)-3-(2,2-difluoroethyl)- 8-(3-(morpholine-4-carbonyl)azetidine-1- carbonyl)-6-oxodecahydropyrrolo[1,2- a][1,5]diazocin-5-yl)carbamoyl)benzo[b] thiophen-5-yl)difluoromethyl)phosphoryl)bis (oxy))bis(methylene) bis(2,2- dimethylpropanoate) | 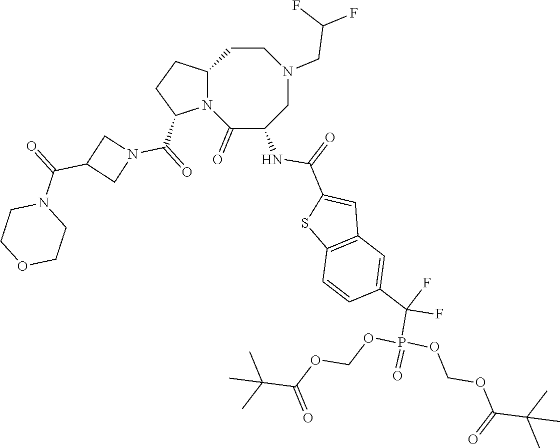 [SMILES: CC(C)(C)C(=O)OCOP(=O)(OCOC(=O)C(C)(C)C)C(F)(F)c1ccc2sc(C(=O)N[C@H]3CN(CC(F)F)CC[C@H]4CC[C@@H](C(=O)N5CC(C(=O)N6CCOCC6)C5)N4C3=O)cc2c1] | 980.0 [M + H]⁺ | ¹H NMR (400 MHZ, CDCl₃) δ 8.09 (s, 1H), 7.95 (m, 1H), 7.85 (m, 1H), 7.65 (m, 1H), 7.50 (s, 1H), 5.76 (m, 1H), 5.73 (m, 1H), 5.69 (m, 1H), 5.65 (m, 1H), 4.93 (s, 1H), 4.71 (s, 1H), 4.59 (s, 1H), 4.45- 4.27 (m, 3H), 4.10 (s, 1H), 3.70 (m, 7H), 3.47 (m, 3H), 3.32 (s, 2H), 3.20 (s, 2H), 3.12- 3.01 (m, 1H), 2.27 (s, 1H), 2.10 (s, 2H), 1.91 (s, 1H), 1.85 (s, 2H), 1.21 (s, 18H). |
| 352 | ((((2-(((5S,8S,10aR)-8-((2-cyano-2- methylpropyl)(methyl)carbamoyl)-3- (2,2-difluoroethyl)-6-oxodecahydropyrrolo[1,2- a][1,5]diazocin-5-yl)carbamoyl)benzo[b] thiophen-5-yl)difluoromethyl)phosphoryl)bis (oxy))bis(methylene) bis(2,2- dimethylpropanoate) | 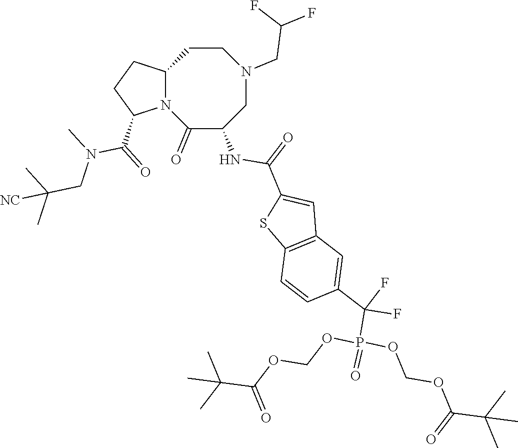 [SMILES: CN(CC(C)(C)C#N)C(=O)[C@@H]1CC[C@@H]2CCN(CC(F)F)C[C@H](NC(=O)c3cc4cc(C(F)(F)P(=O)(OCOC(=O)C(C)(C)C)OCOC(=O)C(C)(C)C)ccc4s3)C(=O)N21] | 904.0 [M − H]⁻ | |
| 353 | ((((2-(((5S,8S,10aR)-3-ethyl-8-(3-(morpholine- 4-carbonyl)azetidine-1-carbonyl)-6- oxodecahydropyrrolo[1,2-a][1,5]diazocin-5- yl)carbamoyl)benzo[b]thiophen-5- yl)difluoromethyl)phosphoryl)bis(oxy)) bis(methylene) bis(2,2-dimethylpropanoate) | 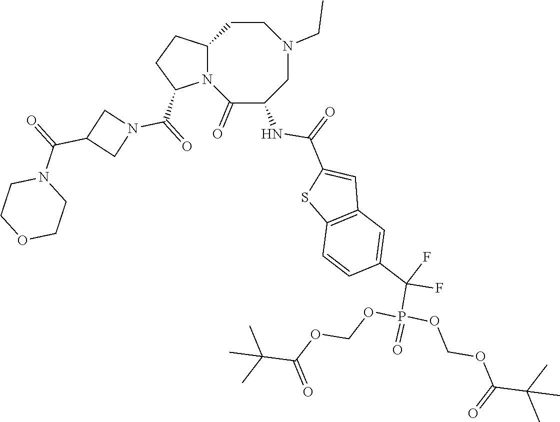 [SMILES: CCN1CC[C@H]2CC[C@@H](C(=O)N3CC(C(=O)N4CCOCC4)C3)N2C(=O)[C@@H](NC(=O)c2cc3cc(C(F)(F)P(=O)(OCOC(=O)C(C)(C)C)OCOC(=O)C(C)(C)C)ccc3s2)C1] | 926.2 [M + H]⁺ | ¹H NMR (600 MHZ, CDCl₃) δ 8.36 (m, 1H), 8.12-7.95 (m, 1H), 7.93-7.80 (m, 2H), 7.58 (m, 1H), 5.89- 5.54 (m, 6H), 4.82- 4.59 (m, 1H), 4.53 (m, 1H), 4.40-4.31 (m, 1H), 4.14-4.03 (m, 1H), 3.68 (m, 8H), 3.27 (m, 4H), 2.28 (m, 2H), 2.11-1.71 (m, 6H), 1.43-1.38 (m, 2H), 1.21 (m, 3H), 1.19 (s Hz, 18H). |

### Additional Compounds

### Synthesis of Cores

### Synthesis of (5R,8S,10aR)-5-((tert-butoxycarbonyl)amino)-6-oxooctahydro-4H-pyrrolo[2,1-d][1,5]thiazocine-8-carboxylic acid 3,3-dioxide

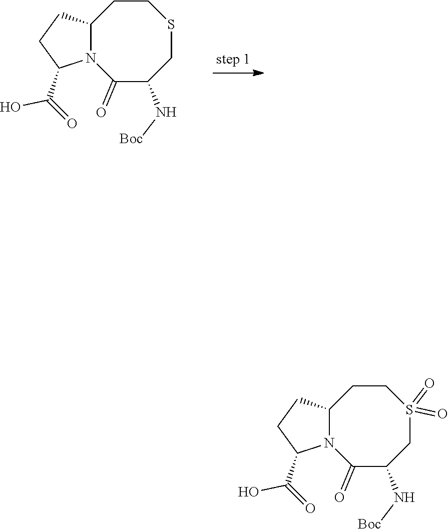 [SMILES: CC(C)(C)OC(=O)N[C@H]1CSCC[C@H]2CC[C@@H](C(=O)O)N2C1=O ; CC(C)(C)OC(=O)N[C@H]1CS(=O)(=O)CC[C@H]2CC[C@@H](C(=O)O)N2C1=O]

### Step 1: Preparation of (5R,8S,10aR)-5-((tert-butoxycarbonyl)amino)-6-oxooctahydro-1H-pyrrolo[2,1-d][1,5]thiazocine-8-carboxylic acid 3,3-dioxide

To a solution of (5R,8S,10aR)-5-((tert-butoxycarbonyl)amino)-6-oxooctahydro-4H-pyrrolo[2,1-d][1,5]thiazocine-8-carboxylic acid (0.20 g, 0.58 mmol, 1.0 eq) in MeOH (2.0 mL) and H₂O (2.0 mL) was added sodium periodate (0.40 g, 0.58 mol, 1.0 eq), the mixture was stirred at 70° C. for 16 hours to give a colorless clear solution. The reaction mixture was filtered and the filtrate was concentrated to afford (5R,8S,10aR)-5-((tert-butoxycarbonyl)amino)-6-oxooctahydro-1H-pyrrolo[2,1-d][1,5]thiazocine-8-carboxylic acid 3,3-dioxide (0.12 g, crude) as a white solid. LCMS (ESI) m/z=321.0

### Synthesis of methyl (4S,7S,9aR)-4-((tert-butoxycarbonyl)amino)-5-oxooctahydro-1H-pyrrolo[1,2-a][1,4]diazepine-7-carboxylate

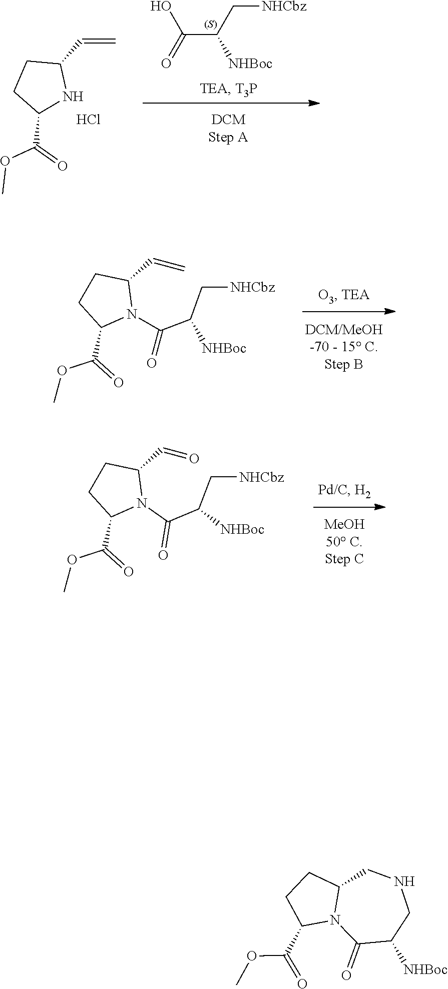

### Step A: methyl (2S,5R)-1-((S)-3-(((benzyloxy)carbonyl)amino)-2-((tert-butoxycarbonyl)amino)propanoyl)-4-vinylpyrrolidine-2-carboxylate

To a solution of methyl (2S,5R)-5-vinylpyrrolidine-2-carboxylate hydrochloride (25.0 g, 130 mmol, 1.00 eq) and (S)-3-(((benzyloxy)carbonyl)amino)-2-((tert-butoxycarbonyl)amino)propanoic acid (66.2 g, 195 mmol, 1.50 eq) in DCM (250 mL) was added TEA (52.8 g, 521 mmol, 72.6 mL, 4.00 eq) and 50% T3P (166 g, 260 mmol, 155 mL, 2.00 eq) drop-wise at 5° C. under N₂. The mixture was warmed to 20° C. and stirred at 20° C. for 12 hrs under N₂. The reaction mixture was diluted with sat. NaHCO₃ (250 mL) and the aqueous phase was extracted with DCM (250 mL*2). The combined organic layers were dried over anhydrous Na₂SO₄, filtered and concentrated under reduced pressure to give a residue. The residue was purified by column chromatography (SiO₂, Petroleum ether:Ethyl acetate=5:1 to 0:1) to afford methyl (2S,5R)-1-((S)-3-(((benzyloxy)carbonyl)amino)-2-((tert-butoxycarbonyl)amino)propanoyl)-5-vinylpyrrolidine-2-carboxylate (55.0 g, 116 mmol, 89% yield) as a yellow oil.

### Step B: methyl (2S,5R)-1-((S)-3-(((benzyloxy)carbonyl)amino)-2-((tert-butoxycarbonyl)amino)propanoyl)-5-formylpyrrolidine-2-carboxylate

O₃ was bubbled through a solution of methyl (2S,5R)-1-((S)-3-(((benzyloxy)carbonyl)amino)-2-((tert-butoxycarbonyl)amino)propanoyl)-5-vinylpyrrolidine-2-carboxylate (27.5 g, 57.8 mmol, 1.00 eq) in DCM (275 mL) and MeOH (27.5 mL) at −70° C. until the color turned to pale blue for 1 hr. 03 was bubbled for 30 mins more before air was bubbled to get rid of excessive 03. After adding TEA (11.7 g, 115 mmol, 16.1 mL, 2.00 eq), the mixture was stirred at 15° C. for 12 hrs. The mixture was concentrated under reduced pressure to give a residue. The residue was purified by column chromatography (SiO₂, Petroleum ether:Ethyl acetate=5:1 to 0:1) to afford methyl (2S,5R)-1-((S)-3-(((benzyloxy)carbonyl)amino)-2-((tert-butoxycarbonyl)amino)propanoyl)-5-formylpyrrolidine-2-carboxylate (35.0 g, 73.3 mmol, 63.3% yield, 2 Batches) as a yellow oil.

### Step C: methyl (4S,7S,9aR)-4-((tert-butoxycarbonyl)amino)-5-oxooctahydro-1H-pyrrolo[1,2-a][1,4]diazepine-7-carboxylate

To a mixture of Pd/C (5.00 g, 10.4 mmol, 10 wt %) in MeOH (300 mL) was added methyl (2S,5R)-1-((S)-3-(((benzyloxy)carbonyl)amino)-2-((tert-butoxycarbonyl)amino)propanoyl)-5-formylpyrrolidine-2-carboxylate (5.00 g, 10.4 mmol, 1.00 eq) at 15° C. The mixture was stirred at 50° C. for 12 hrs under H₂ (15.0 psi) atmosphere. The reaction mixture was filtered and concentrated under reduced pressure to give a residue. The residue was purified by prep-HPLC (column: Welch Xtimate C18 250*70 mm #10 um; mobile phase: [water (NH₄HCO₃)-ACN]; B %: 10%-40%, 20 mins) to afford methyl (4S,7S,9aR)-4-((tert-butoxycarbonyl)amino)-5-oxooctahydro-1H-pyrrolo[1,2-a][1,4]diazepine-7-carboxylate (11.0 g, 33.0 mmol, 45.0% yield, 7 Batches) was obtained as a white solid. ¹H NMR (400 MHz, CDCl₃) δ 5.73 (br d, J=5.1 Hz, 1H), 4.63 (t, J=5.3 Hz, 1H), 4.31-4.14 (m, 1H), 3.83 (br d, J=8.6 Hz, 1H), 3.68 (s, 3H), 3.17 (dd, J=1.5, 13.3 Hz, 1H), 3.00 (d, J=13.9 Hz, 1H), 2.81-2.62 (m, 2H), 2.22-2.03 (m, 2H), 2.01-1.94 (m, 2H), 1.62 (br dd, J=9.5, 12.3 Hz, 1H), 1.36 (s, 9H). LCMS (ESI): m/z=328.2 [M+H]⁺.

### Synthesis of Linkers

### Procedures for Syntheses of Linkers:

The following intermediates in Table 19B were prepared using the described above for synthesis of 5-(((((S)-1-isopropoxy-1-oxopropan-2-yl)amino)(phenoxy)phosphoryl)methyl)benzo[b]thiophene-2-carboxylic acid and utilizing the appropriate utilizing the appropriate starting materials and modifications.

TABLE 19B

| Name | Structure | LCMS |
|---|---|---|
| 5-(fluoro((((S)-1-oxo- 1-propoxypropan-2- yl)amino)(phenoxy) phosphoryl)methyl) benzo[b]thiophene-2- carboxylic acid | 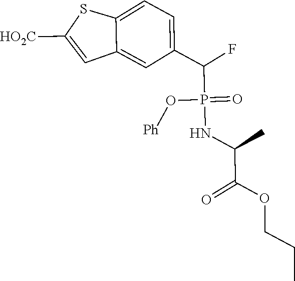 [SMILES: CCCOC(=O)[C@H](C)NP(=O)(Oc1ccccc1)C(F)c1ccc2sc(C(=O)O)cc2c1] | 480.1 [M + H]⁺ |

### Synthesis of ((7-((perfluorophenoxy)carbonyl)quinolin-2-yl)methyl)phosphonic Acid

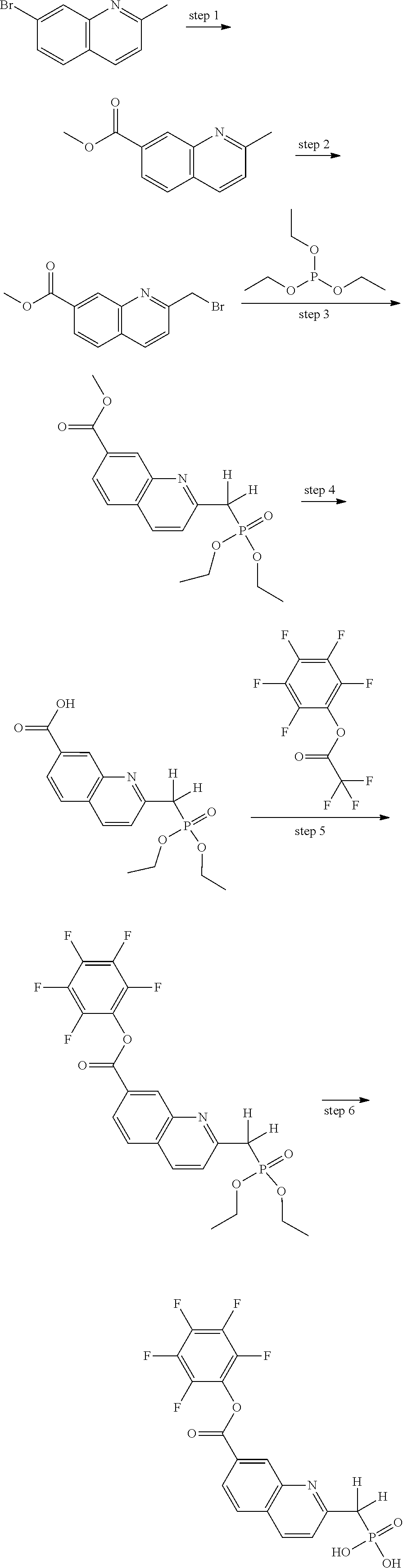

### Step 1: Preparation of methyl 2-methylquinoline-7-carboxylate

To a solution of 7-bromo-2-methylquinoline (1.0 g, 4.5 mmol, 1.0 eq) in MeOH (100 mL) was added TEA (0.91 g, 9.0 mmol, 2.0 eq) and Pd(dppf)Cl₂ (0.33 g, 0.45 mmol, 0.1 eq), and the reaction was placed under an atmosphere of CO (g) at 50 psi. The resulting reaction was stirred for 16 hours at 70° C. The reaction solution was concentrated directly to give a residue. The residue was purified by column chromatography (SiO₂, petroleum ether/ethyl acetate=20:1 to 3:1) was collected and concentrated in vacuo to give methyl 2-methylquinoline-7-carboxylate (0.85 g, 4.2 mmol, 94%) as a white solid. LCMS (ESI) m/z=202.0; ¹H NMR (400 MHz, METHANOL-d₄) δ 8.57 (s, 1H), 8.25 (d, J=8.4 Hz, 1H), 8.04 (d, J=8.4 Hz, 1H), 7.92 (d, J=8.4 Hz, 1H), 7.50 (d, J=8.4 Hz, 1H), 3.98 (s, 3H), 2.73 (s, 3H)

### Step 2: Preparation of methyl 2-(bromomethyl)quinoline-7-carboxylate

To a solution of methyl 2-methylquinoline-7-carboxylate (0.85 g, 4.2 mmol, 1.0 eq) in chloroform (10 mL) was added NBS (0.83 g, 4.6 mmol, 1.1 eq) and AIBN (0.83 g, 4.6 mmol, 0.1 eq), the mixture was stirred at 70° C. for 12 hours. The reaction mixture was quenched by water (30 mL) and extracted with DCM 90 mL (30 mL×3). The combined organic layer was washed with water 30 mL, dried over Na₂SO₄, filtered and concentrated under reduced pressure to give a residue. The residue was purified by column chromatography (SiO₂, Petroleum ether/Ethyl acetate=20:1 to 5:1) was collected and concentrated in vacuo to give methyl 2-(bromomethyl)quinoline-7-carboxylate (0.32 g, 1.1 mmol, 27%) as a white solid. LCMS (ESI) m/z=279.8 ¹H NMR (400 MHz, METHANOL-d₄) δ 8.66 (s, 1H), 8.40 (d, J=8.4 Hz, 1H), 8.14 (d, J=8.4 Hz, 1H), 8.01 (d, J=8.4 Hz, 1H), 7.77 (d, J=8.4 Hz, 1H), 4.77 (s, 2H), 4.00 (s, 3H).

### Step 3: Preparation of methyl 2-((diethoxyphosphoryl)methyl)quinoline-7-carboxylate

A mixture of methyl 2-(bromomethyl)quinoline-7-carboxylate (0.32 g, 1.1 mmol, 1.0 eq) in triethyl phosphite (0.5 mL), the mixture was stirred at 120° C. for 2 hours. The mixture was purified by prep-HPLC (column: Welch Xtimate C18 150×25 mm×5 um; mobile phase: [water (TFA)-ACN]; B %: 20%-50%, 10 min) then lyophilization to give methyl 2-((diethoxyphosphoryl)methyl)quinoline-7-carboxylate (0.35 g, 1.0 mmol, 91%) as a white solid. LCMS (ESI) m/z=338.1 ¹H NMR (400 MHz, DMSO-d₆) δ 8.52 (s, 1H), 8.44 (d, J=8.8 Hz, 1H), 8.14-8.02 (m, 2H), 7.65 (d, J=8.4 Hz, 1H), 4.05-3.99 (m, 4H), 3.94 (s, 3H), 3.78-3.63 (m, 2H), 1.19 (t, J=7.2 Hz, 6H)

### Step 4: Preparation of 2-((diethoxyphosphoryl)methyl)quinoline-7-carboxylic Acid

To a solution of methyl 2-((diethoxyphosphoryl)methyl)quinoline-7-carboxylate (0.35 g, 1.0 mmol, 1.0 eq) in THF (4.0 mL) was added 1M aqueous solution of lithium hydrate hydroxide (2 mL 2.0 mmol, 2 eq), the mixture was stirred at 25° C. for 1 hour. The mixture was adjusted to pH=2 by the addition of 1M aqueous solution of HCl and extracted with ethyl acetate 40 mL. The organic layer was washed with water 120 mL (40 mL×3), dried over Na₂SO₄, filtered and concentrated under reduced pressure to give 2-((diethoxyphosphoryl)methyl)quinoline-7-carboxylic acid (0.3 g, crude) as a white solid. LCMS (ESI) m/z=324.2; ¹H NMR (400 MHz, DMSO-d₆) δ 8.50 (s, 1H), 8.43 (d, J=8.4 Hz, 1H), 8.10-8.02 (m, 2H), 7.68-7.58 (m, 1H), 4.03-3.96 (m, 4H), 3.73-3.66 (m, 2H), 1.19 (t, J=7.2 Hz, 6H)

### Step 5: Preparation of perfluorophenyl 2-((diethoxyphosphoryl)methyl)quinoline-7-carboxylate

To a solution of 2-((diethoxyphosphoryl)methyl)quinoline-7-carboxylic acid (0.3 g, 0.93 mmol, 1.0 eq) in DMA (3.0 mL) was added pyridine (0.37 g, 4.6 mmol, 5 eq), then perfluorophenyl 2,2,2-trifluoroacetate (1.3 g, 4.6 mmol, 5.0 eq) was added dropwise at 0° C. The mixture was stirred at 25° C. for 2 hours to give a yellow solution. The mixture solution was purified by prep-HPLC (column: Phenomenex Luna C18 200×40 mm×10 um; mobile phase: [water (TFA)-ACN]; B %: 46%-76%, 10 min) then lyophilization to give perfluorophenyl 2-((diethoxyphosphoryl)methyl)quinoline-7-carboxylate (0.40 g, 0.82 mmol, 88%.) as a white solid. LCMS (ESI) m/z=490.2; ¹H NMR (400 MHz, DMSO-d₆) δ 8.77 (s, 1H), 8.52 (d, J=8.8 Hz, 1H), 8.30-8.10 (m, 2H), 7.73 (d, J=8.4 Hz, 1H), 4.08-4.01 (m, 4H), 3.83-3.63 (m, 2H), 1.36-1.07 (m, 6H)

### Step 6: Preparation of ((7-((perfluorophenoxy)carbonyl)quinolin-2-yl)methyl)phosphonic Acid

To a solution of perfluorophenyl 2-((diethoxyphosphoryl)methyl)quinoline-7-carboxylate (0.2 g, 0.40 mmol, 1.0 eq) in DCM (5 mL) was added iodotrimethylsilane (0.33 g, 1.6 mmol, 4.0 eq), and the mixture was stirred at 0° C. for 0.5 hour. The mixture was concentrated directly to give a residue which was purified by prep-HPLC (column: Welch Xtimate C18 150×25 mm×5 um; mobile phase: [water (TFA)-ACN]; B %: 20%-50%, 8 min) then lyophilization to give ((7-((perfluorophenoxy)carbonyl) quinolin-2-yl)methyl)phosphonic acid (70 mg, 0.16 mmol, 40%) as a yellow solid. LCMS (ESI) m/z=433.7; ¹H NMR (400 MHz, METHANOL-d₄) δ 8.96-8.80 (m, 1H), 8.72-8.58 (m, 1H), 8.38-8.19 (m, 2H), 8.06-7.87 (m, 1H), 3.33 (d, J=1.2 Hz, 2H).

### Synthesis of ((6-((perfluorophenoxy)carbonyl)isoquinolin-3-yl)methyl)phosphonic Acid

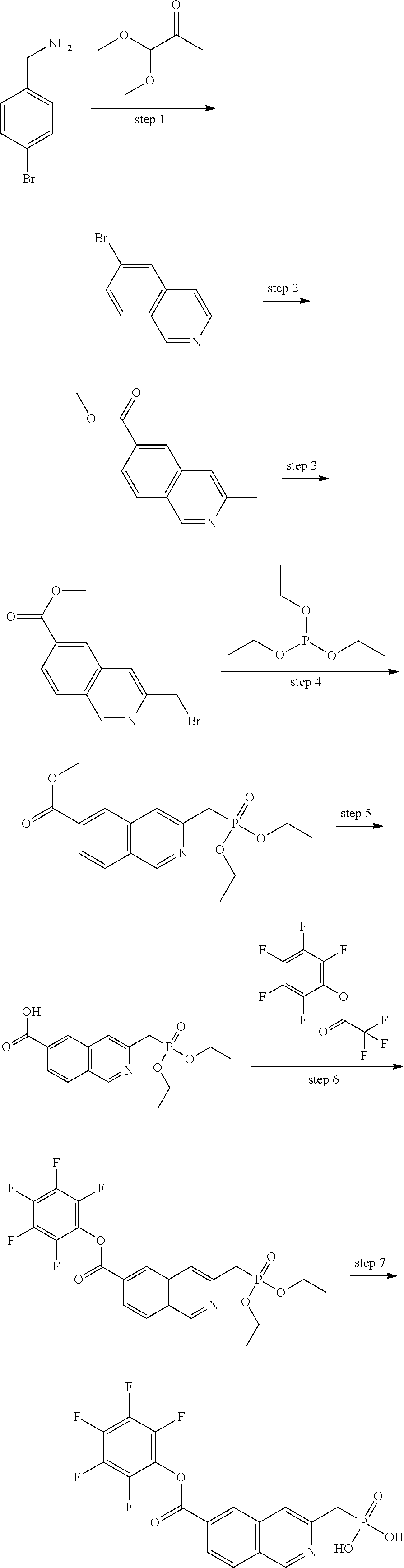

### Step 1: Preparation of 6-bromo-3-methylisoquinoline

To a solution of (4-bromophenyl)methanamine (10 g, 54 mmol, 1.0 eq) in 1,1-dichloroethane (100 mL) was added 1,1-dimethoxypropan-2-one (7.0 g, 59 mmol, 1.1 eq) and magnesium sulfate (10 g, 83 mmol, 1.6 eq), the mixture was stirred at 40° C. for 12 hours. Then sodium (boranylidenemethylidene)azanide (3.9 g, 64 mmol, 1.2 eq) was added, the mixture was stirred at 25° C. for 5 hours. The mixture was filtered. The filtrate was concentrated to give a yellow oil. Then sulfurochloridic acid (6.0 mL) was added dropwise at 0° C. slowly, the reaction mixture was heated to 100° C. for 1 hour, then the reaction mixture was cooled to room temperature and poured into ice water. The mixture was neutralized with 2M NaOH and extracted with ethyl acetate (100 mL×2), the combined organic layers were washed with saturated brine (100 mL×2), dried over Na₂SO₄, filtered and concentrated under reduced pressure to give a residue. The residue was purified by column chromatography (SiO₂, petroleum ether/ethyl acetate=10:1 to 3:1) to give 6-bromo-3-methylisoquinoline (1.8 g, 8.1 mmol, 15% yield) as a white solid. ¹H NMR (400 MHz, CHLOROFORM-d) δ 9.15 (s, 1H), 7.91 (s, 1H), 7.80 (d, J=8.4 Hz, 1H), 7.61 (d, J=8.8 Hz, 1H), 7.39 (s, 1H), 2.71 (s, 3H)

### Step 2: Preparation of methyl 3-methylisoquinoline-6-carboxylate

To a solution of 6-bromo-3-methylisoquinoline (1.5 g, 6.8 mmol, 1.0 eq) in methanol (15 mL) was added triethylamine (1.4 g, 14 mmol, 2 eq) and Pd(dppf)Cl₂ (0.49 g, 0.68 mmol, 0.1 eq) under N₂, the mixture was stirred at 70° C. for 16 hours under CO (50 Psi). The reaction mixture was concentrated under reduced pressure to give a residue. The residue was purified by column chromatography (SiO₂, petroleum ether/ethyl acetate=10:1 to 3:1) to give methyl 3-methylisoquinoline-6-carboxylate (1.4 g, 6.9 mmol, 95% yield) as a white solid. ¹H NMR (400 MHz, CHLOROFORM-d) δ 9.26 (s, 1H), 8.49 (s, 1H), 8.14-8.07 (m, 1H), 8.00 (d, J=8.4 Hz, 1H), 7.59 (s, 1H), 4.01 (s, 3H), 2.74 (s, 3H)

### Step 3: Preparation of methyl 3-(bromomethyl)isoquinoline-6-carboxylate

To a solution of methyl 3-methylisoquinoline-6-carboxylate (0.40 g, 2.0 mmol, 1.0 eq) in CCl₄ (6 mL) was added 1-bromopyrrolidine-2,5-dione (35 mg, 0.20 mmol, 0.1 eq) and 2-[(1E)-2-(1-cyano-1-methylethyl)diazen-1-yl]-2-methylpropanenitrile (0.33 g, 2.0 mmol, 1.0 eq), the mixture was stirred at 65° C. for 12 hours. The reaction mixture was concentrated under reduced pressure to give a residue. The residue was purified by column chromatography (SiO₂, petroleum ether/ethyl acetate=100:1 to 10:1) to give methyl 3-(bromomethyl)isoquinoline-6-carboxylate (0.33 g, 1.20 mmol, 60.0% yield) as a white solid. ¹H NMR (400 MHz, CHLOROFORM-d) δ 9.35 (s, 1H), 8.59 (s, 1H), 8.27-8.20 (m, 1H), 8.12-8.02 (m, 1H), 7.89 (s, 1H), 4.78 (s, 2H), 4.04 (s, 3H)

### Step 4: Preparation of methyl 3-((diethoxyphosphoryl)methyl)isoquinoline-6-carboxylate

A solution of methyl 3-(bromomethyl)isoquinoline-6-carboxylate (0.3 g, 1.1 mmol, 1.0 eq) in triethyl phosphite (3 mL) was stirred at 120° C. for 12 hours. The reaction mixture was purified by reversed phase (TFA) then lyophilization to give methyl 3-((diethoxyphosphoryl)methyl)isoquinoline-6-carboxylate (0.35 g, 1.04 mmol, 97% yield) as a yellow oil. LCMS (ESI) m/z=338.3.

### Step 5: Preparation of 3-((diethoxyphosphoryl)methyl)isoquinoline-6-carboxylic Acid

To a solution of methyl 3-((diethoxyphosphoryl)methyl)isoquinoline-6-carboxylate (0.13 g, 0.39 mmol, 1.0 eq) in THF (0.5 mL) was added sodium hydroxide (31 mg, 0.78 mmol, 2.0 eq) in water (0.5 mL), the mixture was stirred at 25° C. for 2 hours. The mixture was adjusted to pH=2 by 1M aqueous solution of HCl and extracted with ethyl acetate 40 mL. The organic layer was washed with water 120 mL (40 mL×3), dried over Na₂SO₄, filtered and concentrated under reduced pressure to give 3-((diethoxyphosphoryl)methyl)isoquinoline-6-carboxylic acid (0.13 g, crude) as a yellow solid. LCMS (ESI) m/z=324.0.

### Step 6: Preparation of perfluorophenyl 3-((diethoxyphosphoryl)methyl)isoquinoline-6-carboxylate

To a solution of 3-((diethoxyphosphoryl)methyl)isoquinoline-6-carboxylic acid (0.13 g, 0.40 mol, 1.0 eq) in DMF (1.0 mL) was added pyridine (0.13 g, 1.6 mmol, 4.0 eq), then 2,3,4,5,6-pentafluorophenyl 2,2,2-trifluoroacetate (2.3 g, 8.0 mmol, 20 eq) was added at 0° C., and the mixture was stirred at 25° C. for 1 hour to give a yellow solution. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by column chromatography (SiO₂, Petroleum ether/Ethyl acetate=10:1 to 0:1) to give perfluorophenyl 3-((diethoxyphosphoryl)methyl)isoquinoline-6-carboxylate (80 mg, 0.16 mmol, 40% yield) as a yellow solid. LCMS (ESI) m/z=490.2.

### Step 7: Preparation of ((6-((perfluorophenoxy)carbonyl)isoquinolin-3-yl)methyl)phosphonic Acid

To a solution of perfluorophenyl 3-((diethoxyphosphoryl)methyl)isoquinoline-6-carboxylate (80 mg, 0.16 mmol, 1.0 eq) in DCM (1.0 mL) was added iodotrimethylsilane (0.13 g, 0.64 mmol, 4.0 eq) at 0° C. and the mixture was stirred at 0° C. for 10 minutes. The reaction mixture was concentrated under reduced pressure to remove DCM, then was immediately purified by reversed phase HPLC (TFA-modified) then lyophilized to give ((6-((perfluorophenoxy)carbonyl)isoquinolin-3-yl)methyl)phosphonic acid (30 mg, 0.07 mmol, 42% yield) as a red solid. ¹H NMR (400 MHz, METHANOL-d₄) δ 9.69-9.61 (m, 1H), 9.03 (s, 1H), 8.52-8.47 (m, 1H), 8.46-8.42 (m, 1H), 8.35-8.30 (m, 1H), 3.72-3.59 (m, 2H)

### Synthesis of 7-((diethoxyphosphoryl)methyl)-2-naphthoic Acid

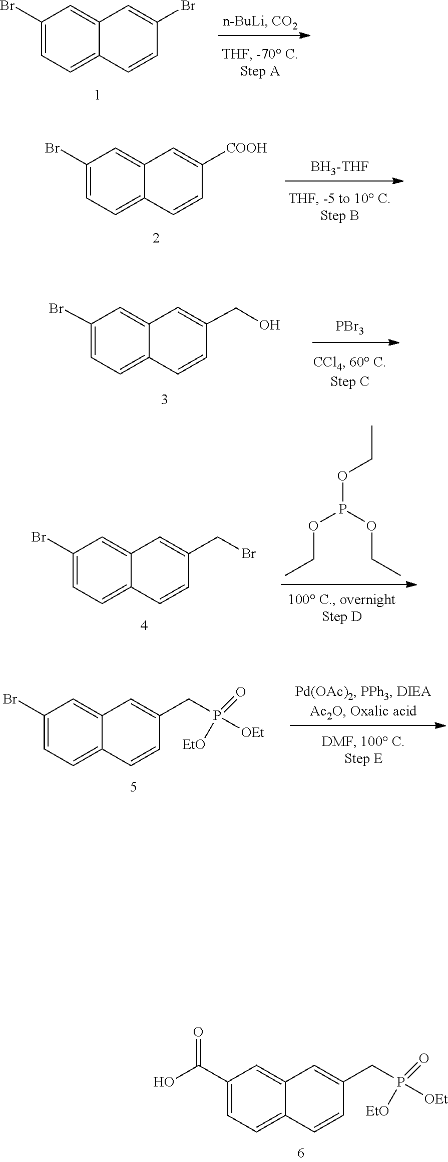

### Step A: Preparation of 7-bromo-2-naphthoic Acid

To a solution of 2,7-dibromonaphthalene (1, 400 g, 1.4 mol, 1.0 eq.) in THF (6000 mL) was added n-BuLi (560 mL, 1.4 mol, 2.5 M in hexane, 1.0 eq.) over a period of 2.5 hrs with a dropwise addition funnel at −70° C. under N₂. Then the reaction mixture was stirred at −70° C. for an additional 1 hr. The reaction was monitored by pipetting out a small amount of crude sample and quenching it with acetone to ensure 1 was consumed completely (observed by TLC). After completion, CO₂(g) was bubbled into the reaction mixture at −70° C. to −60° C. for 30 minutes (3 L/min). The reaction progress was monitored by TLC (DCM:EA=1:1, Rf=0.2). After completion, the reaction mixture was quenched by adding H₂O (5000 mL), the suspension was filtered through a pad of celite and the filter cake was washed with H₂O (1000 mL). The combined filtrates were extracted with PE (3000 mL). The aqueous phase was acidified with 6 N HCl until the pH was adjusted to pH=3. The mixture was filtered and the filter cake was washed with H₂O (600 mL), then dried in vacuum to afford 7-bromo-2-naphthoic acid (2,240 g, 0.96 mol, 68%) as a white solid. LCMS (ESI): m/z=251 [M+H]⁺.

### Step B: Preparation of (7-bromonaphthalen-2-yl)methanol

To a solution of 7-bromo-2-naphthoic acid (2,480 g, 1.92 mol, 1.0 eq.) in THF (1000 mL) was added BH₃/THF (4800 mL, 4.8 mol, 1 M in THF, 2.5 eq.) over a period of 3 hrs at −20° C. to −5° C. using a dropwise addition funnel under N₂. Then the reaction mixture was stirred at −5° C. to 10° C. overnight. The reaction progress was monitored by TLC (DCM:EA=5:1, Rf=0.3). After completion, the reaction mixture was quenched by adding HCl aqueous solution (1N, 400 mL) at 0° C. The mixture was diluted with H₂O (2000 mL) and extracted with EtOAc (2000 mL×2). The combined organic layers were washed with water (3000 mL), brine (2000 mL) and dried over anhydrous Na₂SO₄. The organic layers were concentrated under reduced pressure. The residue was purified by re-crystallization from DCE (2000 mL). The mixture was filtered and the filter cake was washed with PE (200 mL), then dried in vacuum to afford (7-bromonaphthalen-2-yl)methanol (3,405 g, 1.70 mol, 89%) as a white solid.

### Step C: Preparation of 2-bromo-7-(bromomethyl)naphthalene

To a solution of (7-bromonaphthalen-2-yl)methanol (3, 40.0 g, 0.17 mol, 1.0 eq.) in CCl₄ (400 mL) was added PBr₃ (16.1 g, 0.06 mol, 0.35 eq.) at 60° C. After addition, the reaction mixture was stirred at 60° C. for 3 hrs. After completion, the reaction mixture was concentrated under reduced pressure to give a solid. The solid was triturated with PE/EA=8/1 (400 mL) to afford 2-bromo-7-(bromomethyl)naphthalene (4, 48.5 g, 0.16 mol, 96%) as a white solid. ¹H NMR (400 MHz, DMSO-d₆) δ 8.26 (d, J=1.7 Hz, 1H), 8.08-7.90 (m, 3H), 7.75-7.56 (m, 2H), 4.93 (s, 2H).

### Step D: Preparation of diethyl ((7-bromonaphthalen-2-yl)methyl)phosphonate

A solution of 2-bromo-7-(bromomethyl)naphthalene (4, 48.5 g, 0.16 mol, 1.0 eq.) in triethyl phosphite (100 mL) was stirred at 100° C. overnight under nitrogen atmosphere. After completion, the reaction mixture was concentrated under reduced pressure to remove triethyl phosphite. The residue was triturated with IPA (100 mL) to afford diethyl ((7-bromonaphthalen-2-yl)methyl)phosphonate (5, 40.0 g, 0.11 mol, 69%) as a white solid. LCMS (ESI): m/z=357.1 [M+H]⁺. ¹H NMR (400 MHz, CDCl₃) δ 7.95 (s, 1H), 7.76 (d, J=8.4 Hz, 1H), 7.69-7.65 (m, 2H), 7.57-7.39 (m, 2H), 4.13-3.90 (m, 4H), 3.31 (d, J=21.8 Hz, 2H), 1.24 (t, J=7.1 Hz, 6H).

### Step E: Preparation of 7-((diethoxyphosphoryl)methyl)-2-naphthoic Acid

To a solution of diethyl ((7-bromonaphthalen-2-yl)methyl)phosphonate (5, 10.0 g, 27.9 mmol, 1.0 eq.), oxalic acid (10.9 g, 122.0 mmol, 4.4 eq.), Ac₂O (10.5 g, 103.0 mmol, 3.7 eq.) and DIEA (19.3 g, 150.0 mmol, 5.4 eq.) in DMF (60.0 mL) was added Pd(OAc)₂ (625 mg, 2.79 mmol, 0.1 eq.) and PPh₃ (2.2 g, 8.37 mmol, 0.3 eq.). The resulting mixture was stirred at 100° C. overnight under nitrogen atmosphere. After completion, the reaction mixture was cooled to room temperature and acidified with 1 N HCl aqueous solution until the pH was adjusted to pH=3. The mixture was filtered. The filtrate was diluted with H₂O (40 mL) and extracted with EtOAc (200 mL×3). The organic layers were combined, washed with brine (50 mL), dried over anhydrous Na₂SO₄, and concentrated under reduced pressure to afford 7-((diethoxyphosphoryl)methyl)-2-naphthoic acid (6, 8.0 g, 24.8 mmol, 89%) as a yellow solid. LCMS (ESI): m/z=323.2 [M+H]⁺.

### Synthesis of ((7-((allyloxy)carbonyl)naphthalen-2-yl)methyl)phosphonic Acid and ((7-((allyloxy)carbonyl)naphthalen-2-yl)fluoromethyl)phosphonic Acid

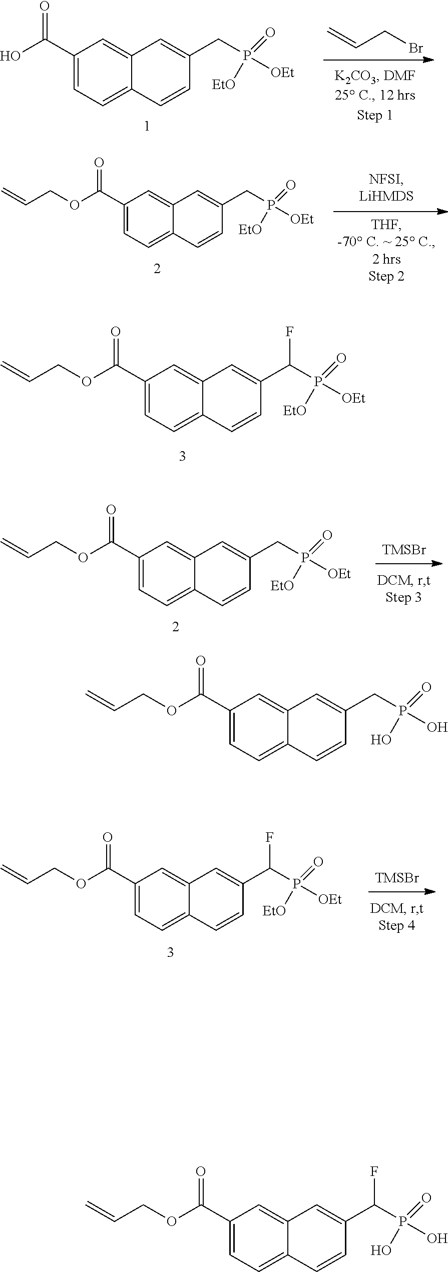

### Step 1: Preparation of allyl 7-((diethoxyphosphoryl)methyl)-2-naphthoate

Four batches of the reactions were carried out in parallel. To a solution of 7-((diethoxyphosphoryl)methyl)-2-naphthoic acid (150 g, 465 mmol, 1.00 eq) and K₂CO₃ (129 g, 931 mmol, 2.00 eq) in DMF (1.50 L) was added allyl bromide (67.6 g, 559 mmol, 1.20 eq) at 25° C. After addition, the reaction mixture was stirred at 25° C. for 12 hrs, at which time LCMS indicated reaction was complete. Four batches of reaction mixture were combined for work-up. The reaction mixture was poured into water (18.0 L) and extracted with ethyl acetate (6.00 L×2). The organic layers were combined, washed with water (6.00 L) and brine (6.00 L), dried over anhydrous Na₂SO₄, and concentrated under reduced pressure to give a crude product. The crude product was purified by silica gel column chromatography (Petroleum ether:Ethyl acetate=50:1 to 0:1, Petroleum ether: Ethyl acetate=0:1, R_{f}=0.50) to give allyl 7-((diethoxyphosphoryl)methyl)-2-naphthoate (568 g, 1.54 mol, 82.7% yield) as a light yellow solid. LCMS (ESI): m/z=363.1 [M+H]⁺. ¹H NMR (400 MHz, CDCl₃) δ 8.59 (s, 1H), 8.07-8.06 (d, J=7.60 Hz, 1H), 7.88-7.84 (m, 3H), 7.58-7.56 (m, 1H), 6.13-6.06 (m, 1H), 5.49-5.44 (m, 1H), 5.35-5.32 (m, 1H), 4.91-4.89 (m, 2H), 4.06-4.02 (m, 4H), 3.36-3.31 (d, J=22.0 Hz 1H), 1.27-1.23 (m, 6H).

### Step 2: Preparation of allyl 7-((diethoxyphosphoryl)fluoromethyl)-2-naphthoate

Eight batches of the reactions were carried out in parallel.

To a solution of allyl 7-((diethoxyphosphoryl)methyl)-2-naphthoate (100 g, 276 mmol, 1.00 eq) in THF (1.50 L) was added NFSI (87.0 g, 276 mmol, 1.00 eq) at 25° C. slowly. The mixture was cooled for −70° C. A solution of LiHMDS (1.00 M, 276 mL, 1.00 eq) was added to the above mixture drop-wise under N₂. After addition, the reaction mixture was stirred at −70° C. for 2 hrs under N₂. The reaction mixture was poured into saturated NH₄Cl aqueous solution (2.00 L) slowly at 0° C. The mixture was extracted with ethyl acetate (1.00 L*2). The organic layers were combined, washed with brine (4.00 L), dried over anhydrous Na₂SO₄ and concentrated under reduced pressure to give a residue. The residue was diluted with ethyl acetate (4.00 L) and filtered. The mother liquor was concentrated under reduced pressure to give a crude yellow oil (779 g). The product was purified by silica gel column chromatography (Petroleum ether:Ethyl acetate=50:1 to 0:1) to give allyl 7-((diethoxyphosphoryl)fluoromethyl)-2-naphthoate (418 g, 1.07 mol, 52.3% yield, 97.4% purity) as yellow oil. LCMS (ESI): m/z=381.1 [M+H]⁺. ¹H NMR (400 MHz, DMSO) δ 8.71 (s, 1H), 8.23 (s, 1H), 8.10-8.04 (m, 3H), 7.74-7.72 (d, J=8.80 Hz, 1H), 6.39-6.26 (m, 1H), 6.14-6.10 (m, 1H), 5.49-5.44 (m, 1H), 5.33-5.30 (m, 1H), 4.89-4.87 (m, 2H), 4.07-4.01 (m, 4H), 1.22-1.15 (m, 6H).

### Step 3: Preparation of ((7-((allyloxy)carbonyl)naphthalen-2-yl)methyl)phosphonic Acid

To a solution of allyl 7-((diethoxyphosphoryl)methyl)-2-naphthoate (4 g, 11.0 mmol, 1.0 eq.) in DCM (40 mL) was added TMSBr (8 mL) at room temperature. After addition, the reaction mixture was stirred at room temperature for 12 hrs. After completion, the reaction mixture was poured into H₂O (30 mL) and the suspension was filtered. The filter cake was dried under vacuum to afford ((7-((allyloxy)carbonyl)naphthalen-2-yl)methyl)phosphonic acid (3.0 g, 9.79 mmol, 89%) as a yellow solid. LCMS (ESI): m/z=307 [M+H]⁺. ¹H NMR (400 MHz, DMSO-d₆) δ 8.56 (s, 1H), 8.04-7.87 (m, 4H), 7.64-7.56 (m, 1H), 6.21-5.96 (m, 1H), 5.53-5.23 (m, 2H), 4.93-4.82 (m, 2H), 3.17 (d, J=21.5 Hz, 2H).

### Step 4: Preparation of ((7-((allyloxy)carbonyl)naphthalen-2-yl)fluoromethyl)phosphonic Acid

To a solution of allyl 7-((diethoxyphosphoryl)fluoromethyl)-2-naphthoate (27 g, 71.1 mmol, 1.0 eq.) in DCM (300 mL) was added TMSBr (50 mL) at room temperature. After addition, the reaction mixture was stirred at room temperature for 12 hrs. After completion, the reaction mixture was concentrated under reduced pressure. The residue was purified by Biotage® C18 column chromatography to afford ((7-((allyloxy)carbonyl)naphthalen-2-yl)fluoromethyl)phosphonic acid (17.6 g, 54.2 mmol, 76%) as a white solid. ¹H NMR (400 MHz, DMSO-d₆) δ 8.67 (s, 1H), 8.16 (s, 1H), 8.10-7.96 (m, 3H), 7.73 (d, J=8.5 Hz, 1H), 6.18-6.04 (m, 1H), 5.89 (dd, J=44.5, 8.6 Hz, 1H), 5.52-5.42 (m, 1H), 5.36-5.26 (m, 1H), 4.88 (d, J=5.4 Hz, 2H). LCMS (ESI): m/z=325.1 [M+H]⁺.

### Chiral Separation of allyl 7-((diethoxyphosphoryl)fluoromethyl)-2-naphthoate

Rac-allyl 7-((diethoxyphosphoryl)fluoromethyl)-2-naphthoate (617 g, 1.62 mol, 1.00 eq) was purified by SFC to give allyl (S)-7-((diethoxyphosphoryl)fluoromethyl)-2-naphthoate (Peak 1) and allyl (R)-7-((diethoxyphosphoryl)fluoromethyl)-2-naphthoate (Peak 2).

Preparative SFC method: Instrument: Waters 350 Preparative SFC. Column: REGIS (S,S) WHELK-O1, 250×50 mm I.D., 10 pm. Mobile phase: A for CO₂ and B for MeOH (Neu). Gradient: B 15% Flow rate: 220 g/min. Back pressure: 100 bar. Column temperature: 35° C. Wavelength: 254 nm. Cycle-time: 7.3 min.

Analytical SFC method: Column: Kromasil (S,S) WHELK-O1, 50×4.6 mm I.D., 3.5 pm. Mobile phase: A for CO₂ and B for MEOH (0.05% DEA). Gradient: B 5 to 40% Flow rate: 3 mL/min. Back pressure: 100 bar. Column temperature: 35° C. Wavelength: 220 nm.

allyl (S)-7-((diethoxyphosphoryl)fluoromethyl)-2-naphthoate (Peak 1, 286 g, 745 mmol, 45.9% yield, >99% ee, RT=1.31 min) was obtained as yellow oil. LCMS (ESI): m/z=381.1 [M+H]⁺.

allyl (R)-7-((diethoxyphosphoryl)fluoromethyl)-2-naphthoate (Peak 2, 288 g, 748 mmol, 46.1% yield, >99% ee, RT=1.52 min) was obtained as yellow oil. LCMS (ESI): m/z=381.1 [M+H]⁺.

Assignment of absolute stereochemical configuration was made by comparison of experimental vibrational circular dichroism (VCD) spectra with theoretical VCD spectra obtained from DFT calculations.

The following intermediates in Table 20 were prepared using the method described above in step 4 for the preparation of ((7-((allyloxy)carbonyl)naphthalen-2-yl)fluoromethyl)phosphonic acid and utilizing the appropriate starting materials and modifications.

TABLE 20

| Name | Structure | LCMS |
|---|---|---|
| (S)-((7-((allyloxy) carbonyl) naphthalen-2- yl)fluoromethyl) phosphonic acid | 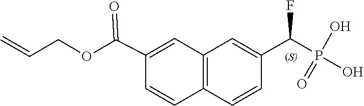 | 325.1 [M + H]⁺ |
| (R)-((7-((allyloxy) carbonyl) naphthalen-2- yl)fluoromethyl) phosphonic acid | 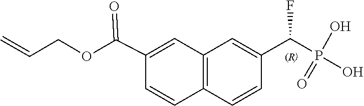 | 325.1 [M + H]⁺ |

### Synthesis of 7-(fluoro((((S)-1-oxo-1-propoxypropan-2-yl)amino)(phenoxy)phosphoryl)methyl)-2-naphthoic Acid

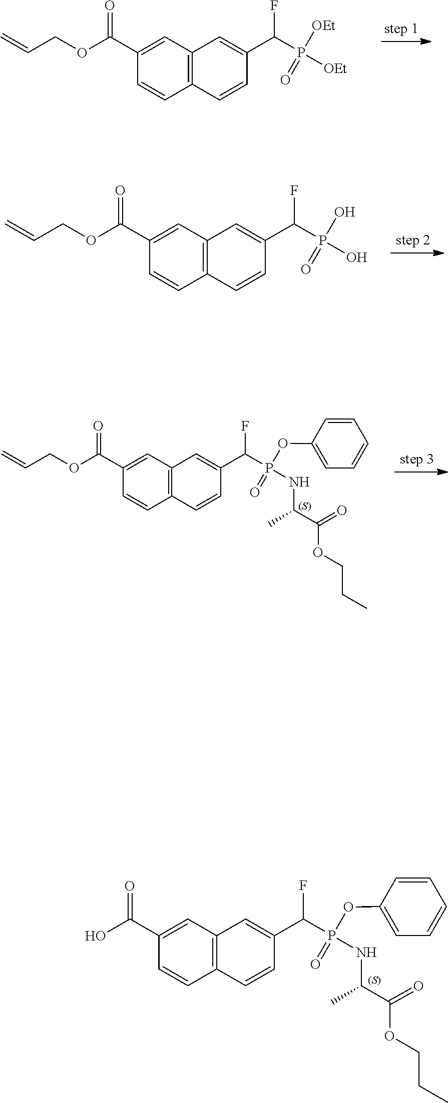

### Step 1: Preparation of ((7-((allyloxy)carbonyl)naphthalen-2-yl)fluromethyl)phosphonic Acid

To a solution of allyl 7-((diethoxyphosphoryl)fluoromethyl)-2-naphthoate (1 g, 2.63 mmol, 1.0 eq.) in DCM (10 mL) were added TMSBr (4.0 g, 26.3 mmol, 10.0 eq.). The mixture was stirred at room temperature for 16 h. After completion, the reaction mixture was concentrated under reduced pressure. The residue was purified by flash column chromatography on silica gel to afford ((7-((allyloxy)carbonyl)naphthalen-2-yl)fluoromethyl)phosphonic acid (680 mg, 2.10 mmol, 80%) as a colorless oil. LCMS (ESI): m/z=325 [M+H]⁺.

### Step 2: Preparation of allyl 7-(fluoro((((S)-1-oxo-1-propoxypropan-2-yl)amino)(phenoxy)phosphoryl)methyl)-2-naphthoate

Oxalyl chloride (1.32 g, 10.5 mmol, 5 eq.) was added dropwise to the solution of afford ((7-((allyloxy)carbonyl)naphthalen-2-yl)fluoromethyl)phosphonic acid (680 mg, 2.10 mmol, 1 eq.) in dry DCM (20 mL) and DMF (0.1 mL) at 25° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (20 mL), then added to a mixture of phenol (197 mg, 2.10 mmol, 1.0 eq.) and TEA (2.12 g, 21.0 mmol, 10 eq.) in anhydrous DCM (20 mL) at −20° C. The reaction was stirred at −20° C. for an additional 1 hr. Then, propyl L-alaninate (550 mg, 4.2 mmol, 2.0 eq.) was added into the above mixture at 0° C. The reaction was stirred at 25° C. for an additional 1 hr. After completion, the reaction was poured into ice-water (10 mL) and extracted with DCM (10 mL×2). The organic layers were combined and washed with brine (10 mL), dried over anhydrous Na₂SO₄, and concentrated under reduced pressure. The residue was purified by Biotage® C18 column chromatography to afford allyl 7-(fluoro((((S)-1-oxo-1-propoxypropan-2-yl)amino)(phenoxy)phosphoryl)methyl)-2-naphthoate (323 mg, 0.63 mmol, 30%) as a brown solid. LCMS (ESI): m/z=514 [M+H]⁺.

### Step 3: Preparation of 7-(fluor((((S)-1-oxo-1-propoxypropan-2-yl)amino)(phenoxy)phosphoryl)methyl)-2-naphthoic Acid

To a solution of allyl 7-(fluoro((((S)-1-oxo-F-propoxypropan-2-yl)amino)(phenoxy)phosphoryl)methyl)-2-naphthoate (323 mg, 0.63 mmol, 1.0 eq) in DCM (10 mL) were added Pd(PPh₃)₄(69 mg, 0.06 mmol, 0.1 eq.) and pyrrolidine (45 mg, 0.63 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 7-(fluoro((((S)-1-oxo-1-propoxypropan-2-yl)amino)(phenoxy)phosphoryl)methyl)-2-naphthoic acid (228 mg, 0.48 mmol, 76%) as a yellow solid. LCMS (ESI): m/z=474 [M+H]⁺.

The following intermediates in Table 21 were prepared using a similar protocol described above for synthesis of (fluoro(6-((perfluorophenoxy)carbonyl)quinolin-3-yl)methyl)phosphonic acid and utilizing the appropriate advanced intermediate(s) as starting material(s).

TABLE 21

| Name | Structure | LCMS |
|---|---|---|
| 7-((1S)-fluoro((((S)-1-oxo-1- propoxypropan-2- yl)amino)(phenoxy)phosphoryl) methyl)-2-naphthoic acid | 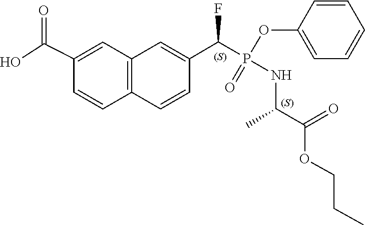 | 474 [M + H]⁺ |
| 7-((1R)-fluoro((((S)-1-oxo-1- propoxypropan-2- yl)amino)(phenoxy)phosphoryl) methyl)-2-naphthoic acid | 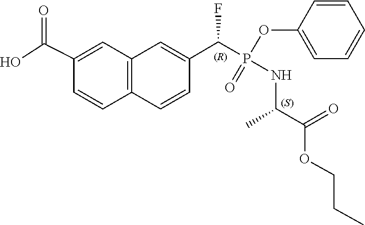 | 474 [M + H]⁺ |
| 7-(((((S)-1-oxo-1-propoxypropan- 2- yl)amino)(phenoxy)phosphoryl) methyl)-2-naphthoic acid | 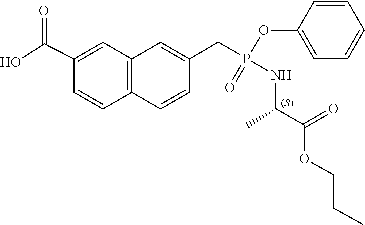 | 456 [M + H]⁺ |

### Synthesis of 3-((diethoxyphosphoryl)methyl)quinoline-6-carboxylic Acid

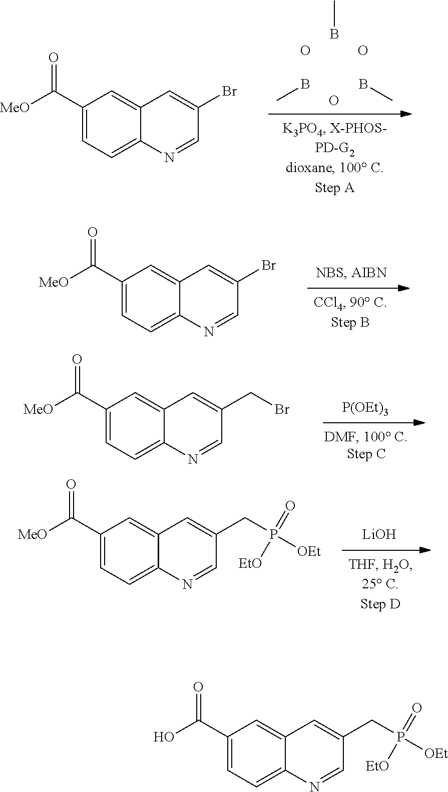

### Step A: Preparation of methyl 3-methylquinoline-6-carboxylate

To a solution of methyl 3-bromoquinoline-6-carboxylate (15.0 g, 56.4 mmol, 1.0 eq) in dioxane (275 mL) was added K₃PO₄ (18.0 g, 84.6 mmol, 1.50 eq) and trimethylboroxine (3.50 M, 32.2 mL, 2.00 eq), and the solution was degassed under vacuum and purged 3× with N₂. XPhos Pd G2 (4.44 g, 5.64 mmol, 0.100 eq) was added and the solution was again degassed under vacuum and purged 3× with N₂. The reaction mixture was heated to 100° C. and stirred for 24 hrs under N₂ at which time LCMS indicated reaction was complete. The mixture was cooled to 25° C. and diluted with EtOAc (275 mL). The suspension was filtered and the cake washed with EtOAc (275 mL), and the combined organics were concentrated under reduced pressure. The resulting crude product was purified by silica gel chromatography (PE:EtOAc=5:1 to 1:1) to afford methyl 3-methylquinoline-6-carboxylate (8.50 g, 40.4 mmol, 71.7% yield) as a yellow solid. ¹H NMR (400 MHz, CDCl₃) δ 8.86 (d, J=2.0 Hz, 1H), 8.52 (d, J=1.6 Hz, 1H), 8.23 (dd, J=1.8, 8.8 Hz, 1H), 8.10 (d, J=9.0 Hz, 1H), 8.01 (s, 1H), 3.99 (s, 3H), 2.55 (s, 3H). LCMS (ESI): m/z=202.1 [M+H]⁺.

### Step B: Preparation of methyl 3-(bromomethyl)quinoline-6-carboxylate

To a solution of methyl 3-methylquinoline-6-carboxylate (2.00 g, 9.51 mmol, 1.0 eq) in CCl₄ (50 mL) was added NBS (2.54 g, 14.3 mmol, 1.50 eq) and AIBN (156 mg, 951 umol, 0.10 eq). The reaction mixture was heated to 90° C. and stirred for 1 h, at which time LCMS indicated formation of the desired product. The mixture was cooled to 25° C., vacuum filtered, and the cake was washed with CCl₄ (50 mL). The combined organics were washed with brine (3×20 mL), dried over Na₂SO₄, and concentrated under reduced pressure. The crude product was purified by silica gel chromatography (PE:EtOAc=10:1 to 1:1) to afford a solid. The product was triturated with petroleum ether (100 mL) for 10 min, and then dried under reduced pressure at 40° C., to afford methyl 3-(bromomethyl)quinoline-6-carboxylate (5.00 g, 16.7 mmol, 41.3% yield) as a yellow solid. ¹H NMR (400 MHz, CDCl₃) δ 9.04 (d, J=2.4 Hz, 1H), 8.59 (d, J=1.8 Hz, 1H), 8.32 (dd, J=1.9, 8.9 Hz, 1H), 8.25 (d, J=2.1 Hz, 1H), 8.16 (d, J=8.9 Hz, 1H), 4.68 (s, 2H), 4.01 (s, 3H). LCMS (ESI): m/z=281.8 [M+H]⁺.

### Step C: Preparation of methyl 3-((diethoxyphosphoryl)methyl)quinoline-6-carboxylate

To a solution of methyl 3-(bromomethyl)quinoline-6-carboxylate (3.00 g, 10.3 mmol, 1.0 eq) in DMF (15 mL) was added P(OEt)₃ (4.59 g, 27.6 mmol, 4.74 mL, 2.7 eq). The mixture was heated to 100° C. and stirred for 3 h at which time LCMS indicated reaction was complete. The mixture was cooled to 25° C., diluted with H₂O (45 mL), and extracted with EtOAc (2×45 mL). The combined organics were washed with brine (2×45 mL), dried over Na₂SO₄, and concentrated under reduced pressure. The crude product was purified by silica gel chromatography (PE:EtOAc=5:1 to 1:5) to afford methyl 3-((diethoxyphosphoryl)methyl)quinoline-6-carboxylate (2.30 g, 6.40 mmol, 62.5% yield) as a white solid. ¹H NMR (400 MHz, CDCl₃) δ 8.90 (s, 1H), 8.55 (d, J=1.3 Hz, 1H), 8.27 (dd, J=0.8, 8.8 Hz, 1H), 8.20 (t, J=2.4 Hz, 1H), 8.11 (d, J=8.8 Hz, 1H), 4.02-4.11 (m, 4H), 3.99 (s, 3H), 3.28-3.37 (m, 2H), 1.26 (t, J=7.1 Hz, 6H). LCMS (ESI): m/z=338.5 [M+H]⁺.

### Step D: Preparation of 3-((diethoxyphosphoryl)methyl)quinoline-6-carboxylic Acid

To a solution of methyl 3-((diethoxyphosphoryl)methyl)quinoline-6-carboxylate (2.30 g, 6.67 mmol, 1.0 eq) in THF (25 mL) was added H₂O (2.5 mL) and LiOH (420 mg, 10.0 mmol, 1.5 eq). The mixture was stirred at ambient temperature for 4 h, at which time LCMS indicated formation of the desired product. The mixture was acidified with 2M HCl to pH 6, and concentrated under reduced pressure. The mixture was then acidified with 1M HCl to pH 5 and filtered. The filter cake was washed with H₂O (10 mL), and then lyophilized to afford 3-((diethoxyphosphoryl)methyl)quinoline-6-carboxylic acid (1.10 g, 3.37 mmol, 50.5% yield) as a white solid. ¹H NMR (400 MHz, DMSO-d₆) δ 13.06-13.36 (m, 1H), 8.91 (t, J=1.8 Hz, 1H), 8.61 (d, J=1.5 Hz, 1H), 8.40 (d, J=2.4 Hz, 1H), 8.18 (dd, J=1.0, 8.8 Hz, 1H), 8.03-8.10 (m, 1H), 3.93-4.07 (m, 4H), 3.47-3.55 (m, 2H), 1.18 (t, J=7.0 Hz, 6H). LCMS (ESI): m/z=324.2 [M+H]⁺.

### Synthesis of (fluoro(6-((perfluorophenoxy)carbonyl)quinolin-3-yl)methyl)phosphonic Acid

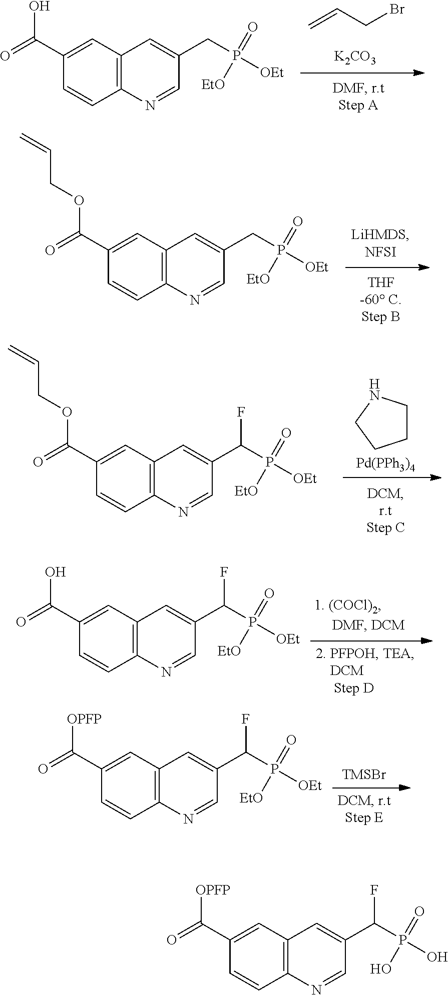

### Step A: Preparation of allyl 3-((diethoxyphosphoryl)methyl)quinoline-6-carboxylate

To a solution of 3-((diethoxyphosphoryl)methyl)quinoline-6-carboxylic acid (900 mg, 2.78 mmol, 1 eq.) and K₂CO₃ (767 mg, 5.56 mmol, 2 eq.) in DMF (5 mL) were added 3-bromoprop-1-ene (402 mg, 3.33 mmol, 1.2 eq.). The resulting mixture was stirred at room temperature for 4 hrs. After completion, the reaction mixture was diluted with H₂O (10 mL), extracted with EtOAc (10 mL×3). The organic layers were combined and washed with brine (10 mL), dried over anhydrous Na₂SO₄, and concentrated under reduced pressure. The residue was purified by flash column chromatography on silica gel to afford allyl 3-((diethoxyphosphoryl)methyl)quinoline-6-carboxylate (912 mg, 2.50 mmol, 90%) as a colorless oil. LCMS (ESI): m/z=364 [M+H]⁺.

### Step B: Preparation of allyl 3-((diethoxyphosphoryl)fluoromethyl)quinoline-6-carboxylate

To a solution of allyl 3-((diethoxyphosphoryl)methyl)quinoline-6-carboxylate (300 mg, 825 μmol, 1 eq.), and NFSI (286 mg, 907 μmol, 1.1 eq.) in THF (10 mL) was added LiHMDS (151 mg, 907 μmol, 1.1 eq.) at −60° C. And the resulting mixture was stirred at −60° C. for 40 min. After completion, the reaction mixture was quenched with NH₄Cl (10 mL, aq. sat.) and extracted with EtOAc (10 mL×2). The organic layers were combined and washed with brine (10 mL), dried over anhydrous Na₂SO₄, and concentrated under reduced pressure. The residue was purified by flash column chromatography on silica gel to afford allyl 3-((diethoxyphosphoryl)fluoromethyl)quinoline-6-carboxylate (50 mg, 131 μmol, 16%) as a yellow oil. LCMS (ESI): m/z=382 [M+H]⁺.

### Step C: Preparation of 3-((diethoxyphosphoryl)fluoromethyl)quinoline-6-carboxylic Acid

To a solution of allyl 3-((diethoxyphosphoryl)fluoromethyl)quinoline-6-carboxylate (50 mg, 131 μmol, 1 eq.) and pyrrolidine (9.31 mg, 131 μmol, 1.0 eq.) in DCM (5 mL) was added Pd(PPh₃)₄(15.1 mg, 13.1 μmol, 0.1 eq.). And the resulting mixture was stirred at room temperature for 30 min. After completion, the reaction mixture was quenched with HCl (10 mL, aq. 1 N) and extracted with DCM (10 mL×2). The organic layers were combined and washed with brine (10 mL), dried over anhydrous Na₂SO₄, and concentrated under reduced pressure to afford 3-((diethoxyphosphoryl)fluoromethyl)quinoline-6-carboxylic acid (40 mg, 117 μmol, 89%) as a yellow solid, which was used in next step directly without further purification.

### Step D: Preparation of perfluorophenyl 3-((diethoxyphosphoryl)fluoromethyl)quinoline-6-carboxylate

Oxalyl chloride (22.2 mg, 175 μmol, 1.5 eq.) was added dropwise to the solution of 3-((diethoxyphosphoryl)fluoromethyl)quinoline-6-carboxylic acid (40 mg, 117 μmol, 1 eq.) in dry DCM (10 mL) and DMF (2 drops) at 25° C. The reaction mixture was stirred at 35° C. for an additional 1 hr. After completion, the excess oxalyl chloride and solvent were removed under reduced pressure. The residue was re-dissolved in anhydrous DCM (10 mL), then added to a mixture of PFPOH (32 mg, 175 μmol, 1.5 eq.) and TEA (59 mg, 585 μmol, 5 eq.) in anhydrous DCM (10 mL) at room temperature. The reaction was stirred at room temperature for an additional 0.5 hrs. After completion, the reaction was diluted with water (10 mL) and extracted with DCM (10 mL×2). The organic layers were combined and washed with brine (10 mL), dried over anhydrous Na₂SO₄, and concentrated under reduced pressure. The residue was purified by flash column chromatography on silica gel to afford perfluorophenyl 3-((diethoxyphosphoryl)fluoromethyl)quinoline-6-carboxylate (46 mg, 91 μmol, 78%) as a yellow oil. LCMS (ESI): m/z=508 [M+H]⁺.

### Step E: Preparation of (fluoro(6-((perfluorophenoxy)carbonyl)quinolin-3-yl)methyl)phosphonic Acid

To a solution of perfluorophenyl 3-((diethoxyphosphoryl)fluoromethyl)quinoline-6-carboxylate (45 mg, 88.7 μmol, 1 eq.) in DCM (5 mL) was added TMSBr (1 mL). The reaction mixture was stirred at room temperature overnight. After completion, the reaction mixture was concentrated under reduced pressure to give (fluoro(6-((perfluorophenoxy)carbonyl)quinolin-3-yl)methyl)phosphonic acid (25.0 mg, 55.4 μmol, 63%) as white solid, which was used in next step directly without further purification. LCMS (ESI): m/z=452 [M+H]⁺.

The following intermediates in Table 22 were prepared using a similar protocol described above for synthesis of (fluoro(6-((perfluorophenoxy)carbonyl)quinolin-3-yl)methyl)phosphonic acid and utilizing the appropriate advanced intermediate(s) as starting material(s).

TABLE 22

| Name | Structure | LCMS |
|---|---|---|
| ((7- ((perfluorophenoxy)carbonyl) naphthalen-2-yl)methyl) phosphonic acid | 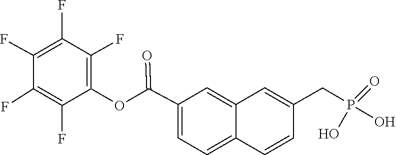 [SMILES: O=C(Oc1c(F)c(F)c(F)c(F)c1F)c1ccc2ccc(CP(=O)(O)O)cc2c1] | 433 [M + H]⁺ |
| (fluoro(7- ((perfluorophenoxy)carbonyl) naphthalen-2-yl)methyl) phosphonic acid | 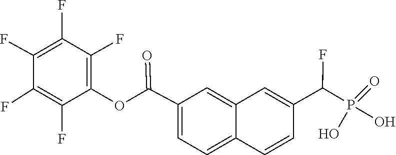 [SMILES: O=C(Oc1c(F)c(F)c(F)c(F)c1F)c1ccc2ccc(C(F)P(=O)(O)O)cc2c1] | 451 [M + H]⁺ |
| (S)-(fluoro(7- ((perfluorophenoxy)carbonyl) naphthalen-2-yl)methyl) phosphonic acid | 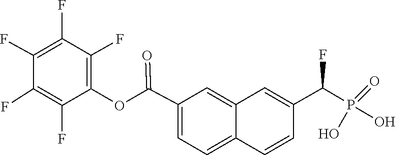 [SMILES: O=C(Oc1c(F)c(F)c(F)c(F)c1F)c1ccc2ccc([C@@H](F)P(=O)(O)O)cc2c1] | 451 [M + H]⁺ |
| (R)-(fluoro(7- ((perfluorophenoxy)carbonyl) naphthalen-2-yl)methyl) phosphonic acid | 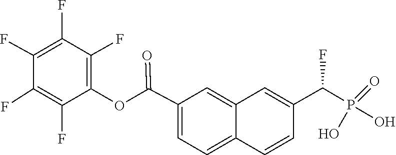 [SMILES: O=C(Oc1c(F)c(F)c(F)c(F)c1F)c1ccc2ccc([C@H](F)P(=O)(O)O)cc2c1] | 451 [M + H]⁺ |

### Synthesis of 4-cyano-7-((diethoxyphosphoryl)methyl)-2-naphthoic Acid

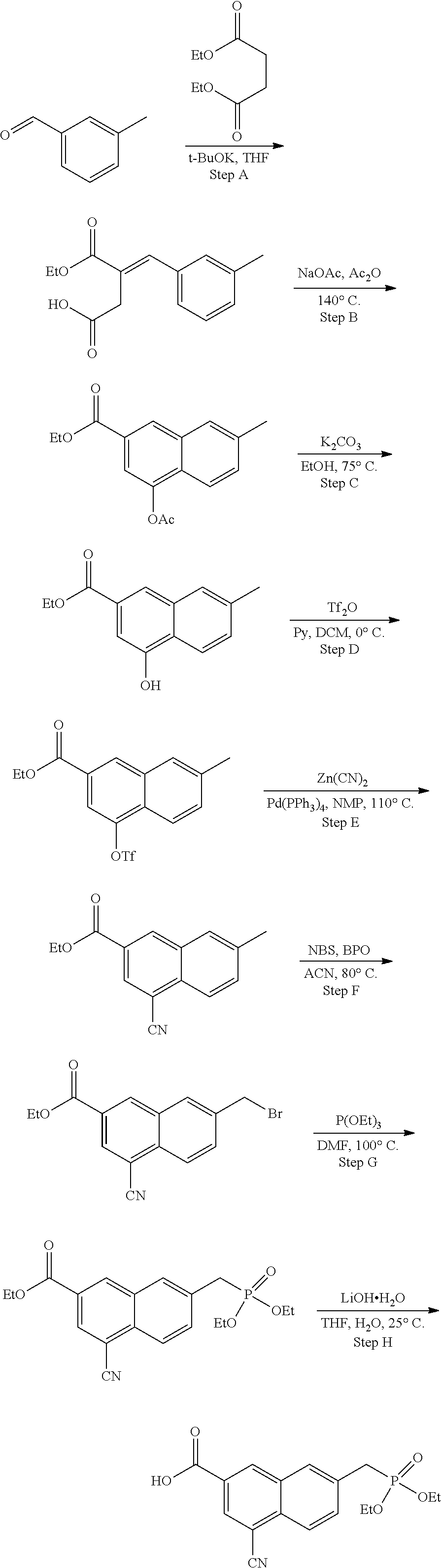

### Step A: Preparation of (E)-3-(ethoxycarbonyl)-4-(m-tolyl)but-3-enoic Acid

To a solution of 3-methylbenzaldehyde (100 g, 832 mmol, 98.0 mL, 1.00 eq) and diethyl succinate (144 g, 832 mmol, 139 mL, 1.00 eq) in THF (2.00 L) was added KOtBu (102 g, 915 mmol, 1.10 eq) at 25° C. The reaction mixture was stirred at 25° C. for 2 hrs, at which time LCMS showed reaction completion. The reaction mixture was poured into water (2.50 L), and then was added i-Pr₂O (1.00 L). The aqueous layer was adjusted to pH=7 with 2N HCl and extracted with Ethyl acetate (1.50 L*3), dried over Na₂SO₄, filtered and concentrated. The crude product was used for next step directly. The (E)-3-(ethoxycarbonyl)-4-(m-tolyl)but-3-enoic acid (168 g, 188 mmol, 22.6% yield) was obtained as a yellow oil. LCMS (ESI): m/z=249.1 [M+H]⁺.

### Step B: Preparation of ethyl 4-acetoxy-7-methyl-2-naphthoate

To a solution of (E)-3-(ethoxycarbonyl)-4-(m-tolyl)but-3-enoic acid (168 g, 178 mmol, 1.00 eq) in Ac₂O (364 g, 3.57 mol, 334 mL, 20.0 eq) was added NaOAc (14.6 g, 178 mmol, 1.00 eq) at 25° C. The solution was degassed and purged with N₂ for 3 times. The reaction mixture was stirred at 140° C. for 2 hrs, at which time LCMS indicated reaction completion. The reaction mixture was poured into water (1.00 L) and extracted with Ethyl acetate (800 mL*3). The combined organic layers were washed with brine (800 mL), dried over Na₂SO₄, filtered and concentrated. The residue was purified by column chromatography (SiO₂, Petroleum ether/Ethyl acetate=1/0 to 20/1, Petroleum ether/Ethyl acetate=5:1, Rf=0.5) to afford ethyl 4-acetoxy-7-methyl-2-naphthoate (54.0 g, 177 mmol, 99.5% yield) as a yellow oil. LCMS (ESI): m/z=273.1 [M+H]⁺.

### Step C: Preparation of ethyl 4-hydroxy-7-methyl-2-naphthoate

To a solution of ethyl 4-acetoxy-7-methyl-2-naphthoate (54.0 g, 177 mmol, 1.00 eq) in EtOH (800 mL) was added K₂CO₃ (122 g, 889 mmol, 5.00 eq) at 25° C. The solution was degassed and purged with N₂ 3 times. The reaction mixture was stirred at 75° C. for 30 mins, at which time LCMS showed reaction was complete. The reaction mixture was cooled 25° C., poured into water (1.50 L), and extracted with Ethyl acetate (1.00 L*3). The combined organic layers were washed with brine (1.00 L), dried over Na₂SO₄, filtered and concentrated. The residue was purified by column chromatography (SiO₂, Petroleum ether/Ethyl acetate=1/0 to 20/1, Petroleum ether/Ethyl acetate=5:1, Rf=0.45), and then purified by prep-HPLC (column: YMC Triart C18 250*50 mm*7 um; mobile phase: [water (NH₄HCO₃)-ACN]; B %: 40%-70%, 20 min) to afford ethyl 4-hydroxy-7-methyl-2-naphthoate (16.0 g, 69.0 mmol, 38.8% yield) as a white solid. LCMS (ESI): m/z=231.1 [M+H]⁺.

### Step D: Preparation of ethyl 7-methyl-4-(((trifluoromethyl)sulfonyl)oxy)-2-naphthoate

To a solution of ethyl 4-hydroxy-7-methyl-2-naphthoate (7.40 g, 31.9 mmol, 1.00 eq) in DCM (148 mL) was added pyridine (5.05 g, 63.8 mmol, 5.15 mL, 2.00 eq) at 25° C. The solution was degassed and purged with N₂ 3 times. A solution of Tf₂O (13.5 g, 47.8 mmol, 7.90 mL, 1.50 eq) in DCM (37.0 mL) was added dropwise at 0° C. and the reaction mixture was stirred for 1 hr, at which time LCMS indicated reaction was complete. The reaction mixture was poured into water (200 mL), and extracted with DCM (200 mL*3). The combined organic layers were washed with brine (200 mL), dried over Na₂SO₄, filtered and concentrated under reduced pressure to afford ethyl 7-methyl-4-(((trifluoromethyl)sulfonyl)oxy)-2-naphthoate (11.6 g, 31.1 mmol, 97.6% yield) was obtained as a yellow solid and was used in the next step without further purification. LCMS (ESI): m/z=363.0 [M+H]⁺.

### Step E: Preparation of ethyl 4-cyano-7-methyl-2-naphthoate

To a solution of ethyl 7-methyl-4-(((trifluoromethyl)sulfonyl)oxy)-2-naphthoate (7.80 g, 21.3 mmol, 1.00 eq) in NMP (78.0 mL) was added Zn(CN)₂ (3.97 g, 33.8 mmol, 2.15 mL, 1.58 eq) and Pd(PPh₃)₄(1.23 g, 1.07 mmol, 0.05 eq) at 25° C. The solution was degassed and purged with N₂ for 3 times. The reaction mixture was stirred at 110° C. for 1 hr, at which time LCMS indicated reaction was complete. The mixture was cooled 25° C., and water (200 mL) and DCM (200 mL) were added at 25-30° C. The insoluble material was removed by filtration through a celite pad. The aqueous layer was separated, and extracted with DCM (200 mL*3). The combined organic layers were washed with brine (200 mL), dried over Na₂SO₄, filtered and concentrated. The residue was purified by column chromatography (SiO₂, Petroleum ether/Ethyl acetate=1/0 to 20/1, Petroleum ether/Ethyl acetate=5:1, Rf=0.5) to afford ethyl 4-cyano-7-methyl-2-naphthoate (4.46 g, 18.4 mmol, 86.3% yield) as a white solid. ¹H NMR (400 MHz, DMSO-d₆) δ 8.73 (s, 1H), 8.44 (d, J=1.6 Hz, 1H), 8.18 (d, J=8.8 Hz, 1H), 7.83 (s, 1H), 7.65 (dd, J=1.6, 8.8 Hz, 1H), 4.48 (q, J=7.2 Hz, 2H), 2.59 (s, 3H), 1.47 (t, J=7.2 Hz, 3H). LCMS (ESI): m/z=240.1 [M+H]⁺.

### Step F. Preparation of ethyl 7-(bromomethyl)-4-cyano-2-naphthoate

To a solution of ethyl 4-cyano-7-methyl-2-naphthoate (4.32 g, 17.8 mmol, 1.00 eq) in ACN (90.0 mL) was added NBS (3.49 g, 19.6 mmol, 1.10 eq) and BPO (432 mg, 1.78 mmol, 0.10 eq) at 25° C. The reaction mixture was stirred at 80° C. for 2 hrs, at which time LCMS indicated reaction was complete. The reaction mixture was cooled 25° C., poured into water (100 mL), and extracted with Ethyl acetate (100 mL*3). The combined organic layers were washed with brine (100 mL), dried over Na₂SO₄, filtered and concentrated to afford ethyl 7-(bromomethyl)-4-cyano-2-naphthoate (2.50 g, 7.43 mmol, 41.6% yield) as a yellow solid, which was used directly in the next step without further purification. LCMS (ESI): m/z=318.0 [M+H]⁺.

### Step G: Preparation of ethyl 4-cyano-7-((diethoxyphosphoryl)methyl)-2-naphthoate

To a solution of ethyl 7-(bromomethyl)-4-cyano-2-naphthoate (2.40 g, 7.13 mmol, 1.00 eq) in DMF (24.0 mL) was added P(OEt)₃ (1.30 g, 7.84 mmol, 1.34 mL, 1.10 eq) at 25° C. The solution was degassed and purged with N₂ three times. The reaction mixture was stirred at 100° C. for 12 hrs, at which time LCMS indicated reaction was complete. The reaction mixture was cooled 25° C., poured into water (100 mL), and extracted with ethyl acetate (100 mL*3). The combined organic layers were washed with brine (100 mL), dried over Na₂SO₄, filtered and concentrated under reduced pressure to afford ethyl 4-cyano-7-((diethoxyphosphoryl)methyl)-2-naphthoate (2.57 g, 5.88 mmol, 82.5% yield) as a yellow solid, which was used directly in the next step without further purification. ¹H NMR (400 MHz, CDCl₃) δ 8.78 (s, 1H), 8.49 (s, 1H), 8.24 (d, J=8.8 Hz, 1H), 7.99 (br s, 1H), 7.77 (br d, J=8.8 Hz, 1H), 4.48 (q, J=7.2 Hz, 2H), 4.06 (t, J=7.2 Hz, 4H), 3.42-3.31 (m, 2H), 1.47 (t, J=7.2 Hz, 3H), 1.27 (t, J=7.2 Hz, 6H). LCMS (ESI): m/z=376.1 [M+H]⁺.

### Step H. Preparation of 4-cyano-7-((diethoxyphosphoryl)methyl)-2-naphthoic Acid

To a solution of ethyl 4-cyano-7-((diethoxyphosphoryl)methyl)-2-naphthoate (2.44 g, 5.58 mmol, 1.00 eq) in THF (25.0 mL) and H₂O (6.50 mL) was added LiOH·H₂O (246 mg, 5.86 mmol, 1.05 eq) at 25° C. The reaction mixture was stirred at 25° C. for 12 hrs, at which time LCMS indicated reaction was complete. The mixture was adjusted pH=6 with 1M HCl, and extracted with DCM (100 mL*3). The combined organic layers were dried over Na₂SO₄, filtered and concentrated under reduced pressure to afford 4-cyano-7-((diethoxyphosphoryl)methyl)-2-naphthoic acid (1.82 g, 4.81 mmol, 86.0% yield) as an off-white solid. ¹H NMR (400 MHz, DMSO-d₆) δ 8.86 (s, 1H), 8.46 (s, 1H), 8.19 (br d, J=1.6 Hz, 1H), 8.14 (d, J=8.8 Hz, 1H), 7.91-7.82 (m, 1H), 4.03-3.93 (m, 4H), 3.55-3.46 (m, 2H), 1.17 (t, J=7.2 Hz, 6H). LCMS (ESI): m/z=348.1 [M+H]⁺.

### Synthesis of 7-((diethoxyphosphoryl)methyl)-4-methoxy-2-naphthoic Acid

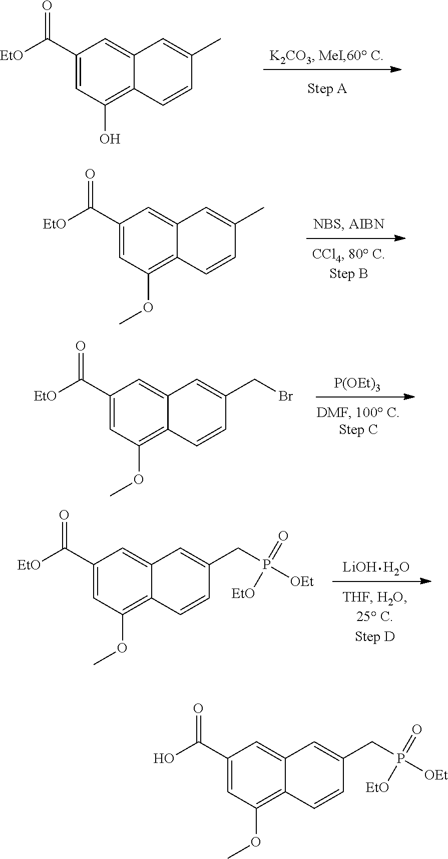

### Step A: Preparation of ethyl 4-methoxy-7-methyl-2-naphthoate

To a solution of ethyl 4-hydroxy-7-methyl-2-naphthoate (4.00 g, 17.2 mmol, 1.00 eq) in ACN (80.0 mL) was added K₂CO₃ (3.58 g, 25.8 mmol, 1.50 eq) at 25° C. The solution was degassed and purged with N₂ for 3 times. MeI (7.35 g, 51.7 mmol, 3.22 mL, 3.00 eq) was added drop-wise to above solution at 20-25° C. The reaction mixture was stirred at 60° C. for 12 hrs, at which time LCMS indicated reaction was complete. The reaction mixture was cooled 20-25° C., poured into saturated KOAc aqueous solution (150 mL), and extracted with DCM (150 mL*3). The combined organic layers were washed with brine (150 mL), dried over Na₂SO₄, filtered and concentrated to afford ethyl 4-methoxy-7-methyl-2-naphthoate (4.21 g, 17.2 mmol, 99.8% yield) as a yellow solid which was used in the next step directly without further purification. ¹H NMR (400 MHz, CDCl₃) δ 8.17 (d, J=8.8 Hz, 1H), 8.14 (s, 1H), 7.68 (s, 1H), 7.42 (dd, J=1.6, 8.4 Hz, 1H), 7.34 (d, J=1.2 Hz, 1H), 4.45 (q, J=7.2 Hz, 2H), 4.06 (s, 3H), 2.53 (s, 3H), 1.46 (t, J=7.2 Hz, 3H). LCMS (ESI): m/z=245.1 [M+H]⁺.

### Step B: Preparation of ethyl 7-(bromomethyl)-4-methoxy-2-naphthoate

To a solution of ethyl 4-methoxy-7-methyl-2-naphthoate (4.12 g, 16.8 mmol, 1.00 eq) in CCl₄ (40.0 mL) was added AIBN (276 mg, 1.69 mmol, 0.10 eq) at 25° C., and the mixture was heated to 80° C. NBS (3.30 g, 18.5 mmol, 1.10 eq) was slowly added at 80-85° C. under N₂. The reaction mixture was stirred at 80° C. for 12 hrs at which time LCMS indicated reaction was complete. The mixture was cooled 25° C., poured into water (100 mL), and extracted with DCM (100 mL*3). The combined organic layers were washed with brine (100 mL), dried over Na₂SO₄, filtered and concentrated under reduced pressure to afford ethyl 7-(bromomethyl)-4-methoxy-2-naphthoate (6.50 g, 16.7 mmol, 99.3% yield) as a yellow solid, which was directly used in the next step without further purification. ¹H NMR (400 MHz, DMSO-d₆) δ 8.27 (d, J=8.8 Hz, 1H), 8.18 (s, 1H), 7.89 (d, J=1.2 Hz, 1H), 7.60 (dd, J=1.6, 8.6 Hz, 1H), 7.42 (d, J=1.2 Hz, 1H), 4.66 (s, 2H), 4.48-4.43 (m, 2H), 4.07-4.04 (m, 3H), 1.46 (t, J=7.2 Hz, 4H). LCMS (ESI): m/z=325.0 [M+H]⁺.

### Step C: Preparation of ethyl 7-((diethoxyphosphoryl)methyl)-4-methoxy-2-naphthoate

To a solution of ethyl 7-(bromomethyl)-4-methoxy-2-naphthoate (6.00 g, 15.4 mmol, 1.00 eq) in DMF (120 mL) was added P(OEt)₃ (2.83 g, 17.0 mmol, 2.92 mL, 1.10 eq) at 25° C. The solution was degassed and purged with N₂ 3 times. The reaction mixture was stirred at 100° C. for 12 hrs at which time LCMS indicated reaction was complete. The mixture was cooled at 25° C., poured into water (600 mL), and extracted with ethyl acetate (400 mL*3). The combined organic layers were washed with brine (500 mL), dried over Na₂SO₄, filtered and concentrated. The residue was purified by column chromatography (SiO₂, Petroleum ether/Ethyl acetate=1/0 to 20/1, Petroleum ether/Ethyl acetate=1:1, Rf=0.4) to afford ethyl 7-(bromomethyl)-4-methoxy-2-naphthoate (3.30 g, 8.62 mmol, 55.7% yield) as a yellow oil. LCMS (ESI): m/z=381.1 [M+H]⁺.

### Step D: Preparation of 7-((diethoxyphosphoryl)methyl)-4-methoxy-2-naphthoic Acid

To a solution of ethyl 7-(bromomethyl)-4-methoxy-2-naphthoate (2.94 g, 7.68 mmol, 1.00 eq) in THF (60.0 mL) and H₂O (15.0 mL) was added LiOH H₂O (338 mg, 8.07 mmol, 1.05 eq) at 25° C. The reaction mixture was stirred at 25° C. for 12 hrs, at which time LCMS indicated formation of the desired product. The mixture was adjusted to pH=5 with 1M HCl and extracted with ethyl acetate (100 mL*3). Combined organic layers were dried over Na₂SO₄, filtered and concentrated to afford 7-((diethoxyphosphoryl)methyl)-4-methoxy-2-naphthoic acid (1.91 g, 5.26 mmol, 68.4% yield) as a yellow solid. ¹H NMR (400 MHz, DMSO-d₆) δ 8.15-8.08 (m, 2H), 7.92 (br d, J=1.6 Hz, 1H), 7.56 (br d, J=8.8 Hz, 1H), 7.33 (s, 1H), 4.02 (s, 3H), 4.00-3.91 (m, 4H), 3.47-3.36 (m, 2H), 1.16 (t, J=7.2 Hz, 6H). LCMS (ESI): m/z=353.1 [M+H]⁺.

### Synthesis of 7-((diethoxyphosphoryl)methyl)-5-fluoro-2-naphthoic Acid

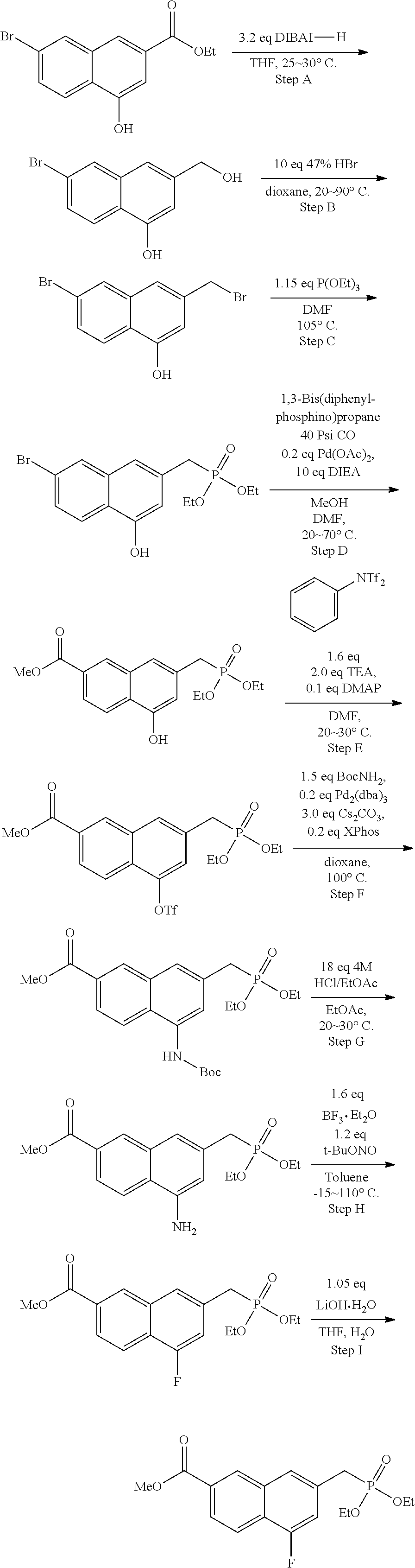

### Step A: Preparation of 6-bromo-3-(hydroxymethyl)naphthalen-1-ol

To a solution of ethyl 7-bromo-4-hydroxy-2-naphthoate (25.0 g, 84.7 mmol, 1.00 eq) in THF (250 mL) was slowly added DIBAl-H (1.00 M, 271 mL, 3.20 eq) under N₂ at 25° C. The reaction mixture was stirred at 25° C. for 4 hrs. The solution was cooled to 0° C. and water (100 mL) was slowly added, and the solution was adjusted to pH=5 by slow addition of 1M HCl (600 mL) at 0° C. The mixture was extracted with ethyl acetate (250 mL*3). The combined organic layers were washed with brine (50 mL), dried over Na₂SO₄, filtered and concentrated under reduced pressure to afford 6-bromo-3-(hydroxymethyl)naphthalen-1-ol (21.0 g, 82.9 mmol, 97.9% yield) as a brown solid, which was used in the next step without further purification. LCMS (ESI): m/z=469.1 [2M−H₂O]⁺.

### Step B: Preparation of 6-bromo-3-(bromomethyl)naphthalen-1-ol

To a solution of 6-bromo-3-(hydroxymethyl)naphthalen-1-ol (21.0 g, 82.9 mmol, 1.00 eq) in dioxane (210 mL) under N₂ was added HBr (47%, 139 g, 829 mmol, 93.8 mL, 10.0 eq) at 25° C. The reaction mixture was heated to 90° C. and stirred for 7 hrs. The solution was then cooled to 25° C. and the mixture was extracted with ethyl acetate (350 mL*3). The combined organic layers were dried over Na₂SO₄, filtered and concentrated under reduced pressure to afford 6-bromo-3-(bromomethyl)naphthalen-1-ol (32.6 g, 67.7 mmol, 81.6% yield) as a brown solid, which was used in the next step without further purification. LCMS (ESI): m/z=301.3 [M-14]+.

### Step C: Preparation of diethyl ((7-bromo-4-hydroxynaphthalen-2-yl)methyl)phosphonate

To a solution of 6-bromo-3-(bromomethyl)naphthalen-1-ol (32.6 g, 67.7 mmol, 1.00 eq) in DMF (100 mL) under N₂ was added triethyl phosphite (12.9 g, 78.1 mmol, 13.4 mL, 1.15 eq) at 25° C. The reaction mixture was slowly heated to 105° C. and stirred for 5 hrs, at which time LCMS indicated the reaction was complete. The solution was then cooled to 25° C., H₂O (270 mL) was added, and the mixture was extracted with ethyl acetate (120 mL*2). The combined organic layers were washed with brine (180 mL), dried over Na₂SO₄, filtered and concentrated under reduced pressure to afford a crude solid. The crude product was triturated with DCM (30 mL) at 25° C., filtered, and washed with DCM (20 mL). The filter cake was then dried under vacuum at 45° C. to afford diethyl ((7-bromo-4-hydroxynaphthalen-2-yl)methyl)phosphonate (9.30 g, 24.0 mmol, 35.5% yield) as a solid. LCMS (ESI): m/z=372.9 [M+H]⁺.

### Step D: Preparation of methyl 7-((diethoxyphosphoryl)methyl)-5-hydroxy-2-naphthoate

To a solution of diethyl ((7-bromo-4-hydroxynaphthalen-2-yl)methyl)phosphonate (9.30 g, 24.0 mmol, 1.00 eq) in DMF (93 mL) and MeOH (93 mL) was added 1,3-Bis(diphenylphosphino)propane (1.99 g, 4.81 mmol, 0.20 eq), DIEA (24.3 g, 240 mmol, 33.5 mL, 10.0 eq), and Pd(OAc)₂ (1.08 g, 4.81 mmol, 0.20 eq) under Ar at 25° C. The mixture was degassed and purged with CO 3 times, and placed under CO atmosphere (40 psi). The reaction mixture was heated to 70° C. and stirred for 20 hrs. After cooling, LCMS indicated the reaction was complete. The mixture was filtered, the filter cake washed with MeOH (100 mL), and the filtrate was concentrated under reduced pressure. The resulting residue was partitioned with water (400 mL) and EtOAc (300 mL*3). The combined organic layers were washed with brine (300 mL), dried over Na₂SO₄, filtered and concentrated under reduced pressure to afford methyl 7-((diethoxyphosphoryl)methyl)-5-hydroxy-2-naphthoate (8.30 g, 23.3 mmol, 96.8% yield) as a brown solid, which was used in the next step without further purification. LCMS (ESI): m/z=352.9 [M+H]⁺.

### Step E: Preparation of methyl 7-((diethoxyphosphoryl)methyl)-5-(((trifluoromethyl)sulfonyl)oxy)-2-naphthoate

To a solution of methyl 7-((diethoxyphosphoryl)methyl)-5-hydroxy-2-naphthoate (8.30 g, 23.3 mmol, 1.00 eq) in DCM (180 mL) was added triethylamine (4.72 g, 46.6 mmol, 6.49 mL, 2.0 eq), 4-DMAP (284 mg, 2.33 mmol, 0.10 eq), and N-Phenyltriflamide (13.3 g, 37.3 mmol, 1.60 eq) under N₂ atmosphere at 25° C. The reaction mixture was stirred for 5 hrs, at which time LCMS indicated the reaction was complete. The mixture was diluted with DCM (200 mL) and water (300 mL). The layers were separated and extracted with DCM (200 mL). The combined organic layers were washed with brine (200 mL), dried over Na₂SO₄, filtered and concentrated under reduced pressure to afford methyl 7-((diethoxyphosphoryl)methyl)-5-(((trifluoromethyl)sulfonyl)oxy)-2-naphthoate (14.6 g, 21.2 mmol, 90.9% yield) as a brown oil, which was used in the next step without further purification. LCMS (ESI): m/z=485.0 [M+H]⁺.

### Step F. Preparation of methyl 5-((tert-butoxycarbonyl)amino)-7-((diethoxyphosphoryl)methyl)-2-naphthoate

To a solution of methyl 7-((diethoxyphosphoryl)methyl)-5-(((trifluoromethyl)sulfonyl)oxy)-2-naphthoate (14.6 g, 21.2 mmol, 1.00 eq) and BocNH₂ (3.73 g, 31.8 mmol, 1.50 eq) in dioxane (200 mL) was added Cs₂CO₃ (20.7 g, 63.6 mmol, 3.00 eq), XPhos (2.02 g, 4.24 mmol, 0.20 eq) and Pd₂(dba)₃ (3.89 g, 4.24 mmol, 0.20 eq) at 25° C. The solution was degassed and purged with N₂ for 3 times. The mixture was heated to 100° C. and stirred for 4 hrs, at which time LCMS indicated reaction was complete. The mixture was cooled to 25° C., filtered through celite, and concentrated under reduced pressure. The residue was purified by silica gel chromatography (PE/EtOAc 2:1 to 1:2) to afford methyl 5-((tert-butoxycarbonyl)amino)-7-((diethoxyphosphoryl)methyl)-2-naphthoate (6.23 g, 10.1 mmol, 48.0% yield) as a red oil. LCMS (ESI): m/z=452.1 [M+H]⁺.

### Step G: Preparation of methyl 5-amino-7-((diethoxyphosphoryl)methyl)-2-naphthoate

To a solution of methyl 5-((tert-butoxycarbonyl)amino)-7-((diethoxyphosphoryl)methyl)-2-naphthoate (6.23 g, 10.1 mmol, 1.00 eq) in EtOAc (25 mL) was added HCl/EtOAc (4.00 M, 45.8 mL, 18.0 eq) at 10-15° C. The reaction mixture was stirred at 25° C. for 1 hr, at which time LCMS indicated reaction was complete. The mixture was adjusted to pH=8 by addition of sat. aq. NaHCO₃, and was then extracted with ethyl acetate (60 mL*3). The combined organic layer were washed with brine (30 mL), dried over Na₂SO₄, filtered and concentrated to afford methyl 5-amino-7-((diethoxyphosphoryl)methyl)-2-naphthoate (3.23 g, 6.50 mmol, 63.8% yield) as a brown solid, which was used directly in the next step without further purification. LCMS (ESI): m/z=351.9 [M+H]⁺.

### Step H. Preparation of methyl 7-((diethoxyphosphoryl)methyl)-5-fluoro-2-naphthoate

To a solution of methyl 5-amino-7-((diethoxyphosphoryl)methyl)-2-naphthoate (3.23 g, 6.50 mmol, 1.00 eq) in DCM (64 mL) was slowly added t-BuONO (804 mg, 7.80 mmol, 0.927 mL, 1.20 eq) at −15˜−10° C. The mixture was stirred for 30 min, and a solution of BF₃·Et₂O (1.48 g, 10.4 mmol, 1.28 mL, 1.60 eq) in DCM (16 mL) was slowly added at −15˜−10° C., and the mixture was stirred for 1 hour at 0° C. The mixture was concentrated at 20° C. and then PhMe (64 mL) was added at 20-25° C. under N₂. The mixture was heated to 110° C. and stirred for 12 hrs. LCMS indicated reaction completion and the mixture was cooled to 25° C. and concentrated under reduced pressure at 50° C. The residue was purified by prep HPLC (column: Phenomenex luna C18 250*50 mm*10 um; mobile phase: [water(HCl)-ACN]; B %: 40%-70%, 20 min) to afford methyl 7-((diethoxyphosphoryl)methyl)-5-fluoro-2-naphthoate (1.20 g, 2.96 mmol, 45.5% yield) as a colorless oil. ¹H NMR (400 MHz, CDCl₃) δ 8.55 (s, 1H) 8.10 (t, J=9.6 Hz, 2H) 7.64 (d, J=2.8 Hz, 1H) 8.10 (t, J=9.6 Hz, 2H) 7.36-7.32 (m, 1H) 4.10-4.03 (m, 4H) 4.00 (s, 3H) 3.34 (d, J=22.0 Hz, 2H) 1.27 ((t, J=7.2 Hz, 6H). LCMS (ESI): m/z=354.9 [M+H]⁺.

### Step I: Preparation of 7-((diethoxyphosphoryl)methyl)-5-fluoro-2-naphthoic Acid

To a solution of methyl 7-((diethoxyphosphoryl)methyl)-5-fluoro-2-naphthoate (1.20 g, 2.96 mmol, 1.00 eq) in THF (25.0 mL) and H₂O (2.5 mL) was added LiOH·H₂O (130 mg, 3.11 mmol, 1.05 eq). The reaction mixture was stirred at 25° C. for 16 hrs, at which time LCMS indicated formation of desired product. The solution was cooled to 0° C., adjusted to pH=5 with 1M HCl (3.3 mL), and was concentrated under reduced pressure at 45° C. The resulting residue was partitioned with water (20 mL) and 2-Me-THF (30 mL*3). The combined organic layers were washed with brine (20 mL), dried over Na₂SO₄, filtered and concentrated. The crude product was triturated with 2-Me-THF/PE (1:20, 20 mL), filtered, and washed with PE (10 mL). The filter cake was dried under vacuum at 45° C. to afford 7-((diethoxyphosphoryl)methyl)-5-fluoro-2-naphthoic acid (1.18 g, 3.40 mmol, 82.0% yield) as a yellow solid. ¹H NMR (400 MHz, CDCl₃) δ 8.58 (s, 1H) 8.04 (q, J=8.8, 22.8 Hz, 2H) 7.75 (d, J=3.2 Hz, 1H) 7.22 (d, J=11.2 Hz, 1H) 4.18-4.12 (m, 1H) 3.38 (d, J=22.0 Hz, 2H) 1.43-1.29 (m, 6H). LCMS (ESI): m/z=341.0 [M+H]⁺.

### Synthesis of 5-cyano-7-((diethoxyphosphoryl)methyl)-2-naphthoic Acid

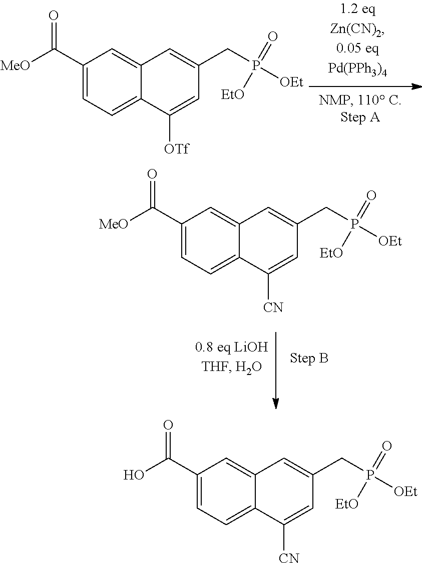

### Step A: Preparation of methyl 5-cyano-7-((diethoxyphosphoryl)methyl)-2-naphthoate

To a solution of methyl 7-((diethoxyphosphoryl)methyl)-5-(((trifluoromethyl)sulfonyl)oxy)-2-naphthoate (4.26 g, 6.44 mmol, 1.00 eq) in NMP (45 mL) was added Zn(CN)₂ (0.960 g, 8.18 mmol, 0.518 mL, 1.27 eq) and Pd(PPh₃)₄(0.371 g, 0.321 mmol, 0.05 eq) at 25° C. The solution was degassed and purged with N₂ for 3 times. The reaction mixture was stirred at 110° C. for 2 hr, at which time LCMS indicated reaction was complete. The mixture was cooled 25° C., and water (150 mL) was added. The mixture was extracted with EtOAc (150 mL*3). The combined organic layers were washed with brine (150 mL), dried over Na₂SO₄, filtered and concentrated under reduced pressure. The residue was purified by column chromatography (SiO₂, Petroleum ether/Ethyl acetate=2:1 to 1:2, Petroleum ether/Ethyl acetate=1:2, R_{f}=0.3) to afford methyl 5-cyano-7-((diethoxyphosphoryl)methyl)-2-naphthoate (2.40 g, 5.89 mmol, 91.5% yield) as a brown oil. LCMS (ESI): m/z=362.1 [M+H]⁺.

### Step B: Preparation of 5-cyano-7-((diethoxyphosphoryl)methyl)-2-naphthoic Acid

To a solution of methyl 5-cyano-7-((diethoxyphosphoryl)methyl)-2-naphthoate (2.40 g, 5.89 mmol, 1.00 eq) in THF (24.0 mL) and H₂O (2.4 mL) was added LiOH·H₂O (197 mg, 4.71 mmol, 0.80 eq). The reaction mixture was stirred at 25° C. for 13 hrs, at which time LCMS indicated formation of desired product. The solution was cooled to 0° C., adjusted to pH=5 with 4M HCl/EtOAc (0.25 mL), and was concentrated under reduced pressure at 40° C. The resulting residue was purified by prep HPLC (column: Phenomenex luna C18 250*50 mm*10 um; mobile phase: [water(HCl)-ACN]; B %: 25%-55%, 20 min) to afford 5-cyano-7-((diethoxyphosphoryl)methyl)-2-naphthoic acid (1.01 g, 2.86 mmol, 38.8% yield) as a white solid. ¹H NMR (400 MHz, CDCl₃) δ 8.64 (s, 1H) 8.24 (d, J=2.2 Hz, 1H) 8.16-8.20 (m, 1H) 8.06-8.10 (m, 1H) 7.88-7.95 (m, 1H) 4.12-4.26 (m, 4H) 3.29-3.49 (m, 2H) 1.35 (t, J=7.2 Hz, 6H). LCMS (ESI): m/z=348.1 [M+H]⁺.

### Synthesis of ((5-cyano-7-((perfluorophenoxy)carbonyl)naphthalen-2-yl)methyl)phosphonic Acid

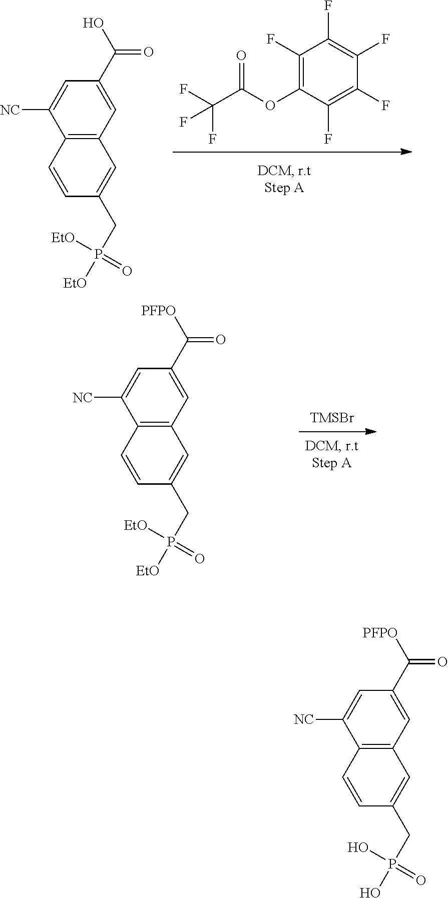

### Step A: Preparation of perfluorophenyl 4-cyano-7-((diethoxyphosphoryl)methyl)-2-naphthoate

To a solution of 4-cyano-7-((diethoxyphosphoryl)methyl)-2-naphthoic acid (500 mg, 1.43 mmol, 1 eq.) and pyridine (565 mg, 7.15 mmol, 5 eq.) in DCM (10 mL) was added perfluorophenyl 2,2,2-trifluoroacetate (599 mg, 2.14 mmol, 1.5 eq.) dropwise at 5° C. The reaction mixture was warmed up to room temperature and stirred overnight. After completion, the reaction mixture was concentrated under reduced pressure. The residue was purified by flash column chromatography on silica gel to afford perfluorophenyl 4-cyano-7-((diethoxyphosphoryl)methyl)-2-naphthoate (600 mg, 1.16 mmol, 82%) as a colorless oil. LCMS (ESI): m/z=514 [M+H]⁺.

### Step B: Preparation of ((5-cyano-7-((perfluorophenoxy)carbonyl)naphthalen-2-yl)methyl)phosphonic Acid

To a solution of perfluorophenyl 4-cyano-7-((diethoxyphosphoryl)methyl)-2-naphthoate (600 mg, 1.16 mmol, 1 eq.) in DCM (10 mL) was added TMSBr (2 mL). The reaction mixture was stirred at room temperature overnight. After completion, the reaction mixture was concentrated under reduced pressure to give ((5-cyano-7-((perfluorophenoxy)carbonyl)naphthalen-2-yl)methyl)phosphonic acid (500 mg, quant.) as a white solid, which was used in next step directly without further purification. LCMS (ESI): m/z=458 [M+H]⁺.

The following intermediates in Table 23 were prepared using a similar protocol described above for synthesis of ((5-cyano-7-((perfluorophenoxy)carbonyl)naphthalen-2-yl)methyl)phosphonic acid and utilizing the appropriate advanced intermediate(s) as starting material(s).

TABLE 23

| Name | Structure | LCMS |
|---|---|---|
| ((4-cyano-7- ((perfluorophenoxy)carbonyl) naphthalen-2-yl)methyl) phosphonic acid | 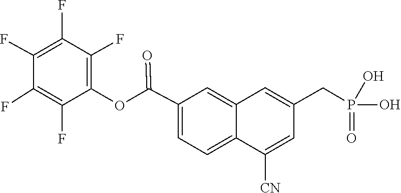 [SMILES: N#Cc1cc(CP(=O)(O)O)cc2cc(C(=O)Oc3c(F)c(F)c(F)c(F)c3F)ccc12] | 458.0 [M + H]⁺ |
| ((5-methoxy-7- ((perfluorophenoxy)carbonyl) naphthalen-2-yl)methyl) phosphonic acid | 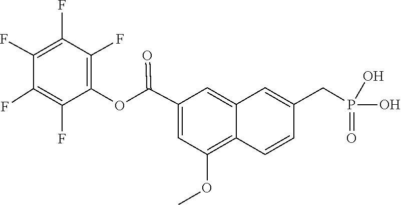 [SMILES: COc1cc(C(=O)Oc2c(F)c(F)c(F)c(F)c2F)cc2cc(CP(=O)(O)O)ccc12] | 463.0 [M + H]⁺ |

### Synthesis of ((4-fluoro-7-((perfluorophenoxy)carbonyl)naphthalen-2-yl)methyl)phosphonic Acid

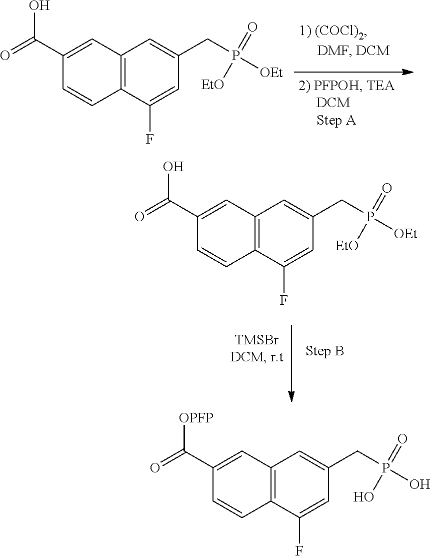

### Step A: Preparation of perfluorophenyl 7-((diethoxyphosphoryl)methyl)-5-fluoro-2-naphthoate

Oxalyl chloride (926 mg, 7.35 mmol, 5 eq.) was added dropwise to the solution of 7-((diethoxyphosphoryl)methyl)-5-fluoro-2-naphthoic acid (500 mg, 1.47 mmol, 1 eq.) in dry DCM (10 mL) and DMF (2 drops) at 25° C. The reaction mixture was stirred at 35° C. for an additional 1 hr. After completion, the excess oxalyl chloride and solvent were removed under reduced pressure. The residue was re-dissolved in anhydrous DCM (10 mL), then added to a mixture of PFPOH (407 mg, 2.21 mmol, 1.5 eq.) and TEA (742 mg, 7.35 mmol, 5 eq.) in anhydrous DCM (10 mL) at room temperature. The reaction was stirred at room temperature for an additional 0.5 hrs. After completion, the reaction was diluted with water (10 mL) and extracted with DCM (10 mL×2). The organic layers were combined and washed with brine (10 mL), dried over anhydrous Na₂SO₄, and concentrated under reduced pressure. The residue was purified by flash column chromatography on silica gel to afford perfluorophenyl 7-((diethoxyphosphoryl)methyl)-5-fluoro-2-naphthoate (655 mg, 1.29 mmol, 88%) as a yellow oil. LCMS (ESI): m/z=507 [M+H]⁺.

### Step B: Preparation of ((4-fluoro-7-((perfluorophenoxy)carbonyl)naphthalen-2-yl)methyl)phosphonic Acid

To a solution of perfluorophenyl 7-((diethoxyphosphoryl)methyl)-5-fluoro-2-naphthoate (655 mg, 1.29 mmol, 1 eq.) in DCM (15 mL) was added TMSBr (3 mL). The reaction mixture was stirred at room temperature overnight. After completion, the reaction mixture was concentrated under reduced pressure to give ((4-fluoro-7-((perfluorophenoxy)carbonyl)naphthalen-2-yl)methyl)phosphonic acid (520 mg, quant.) as yellow solid, which was used in next step directly without further purification. LCMS (ESI): m/z=449.3 [M−H]⁻.

### Synthesis of Building Blocks

### Synthesis of 4-(4-azaspiro[2.4]heptan-6-yl)pyridin-2(1H)-one and 1-methyl-4-(4-azaspiro[2.4]heptan-6-yl)pyridin-2(1H)-one

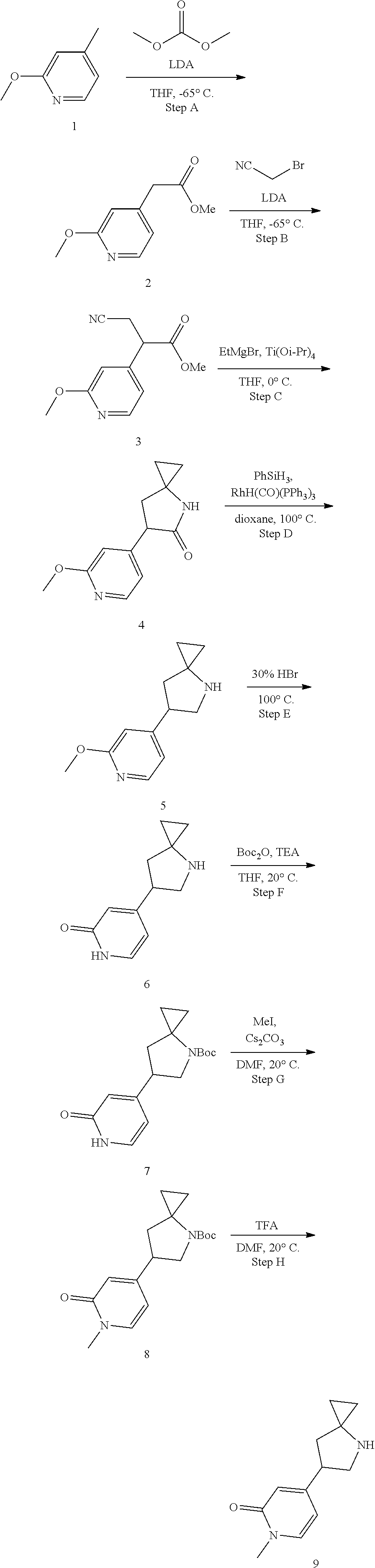

### Step A: Preparation of methyl 2-(2-methoxypyridin-4-yl)acetate

To a solution of 2-methoxy-4-methylpyridine (1, 50 g, 406 mmol, 1.0 eq.) in THF (500 mL) was added dropwise LDA (243 mL, 485 mmol, 2M, 1.2 eq.) at −65° C. The reaction solution was stirred at −65° C. for 1 hr and dimethyl carbonate (40 g, 445 mmol, 1.1 eq.) was added into the reaction mixture. After addition, the mixture was stirred at −65° C. for an additional 1 hr. After completion, the reaction mixture was quenched with saturated NH₄Cl (aq.) solution (500 mL) and extracted with EtOAc (500 mL×3). The organic layers were combined and washed with brine (500 mL), dried over anhydrous Na₂SO₄ and concentrated under reduced pressure. The residue was purified by flash column chromatography on silica gel to afford methyl 2-(2-methoxypyridin-4-yl)acetate (2, 35 g, 192 mmol, 47%) as a yellow oil. LCMS (ESI): m/z=182.2 [M+H]⁺. ¹H NMR (400 MHz, CDCl₃) δ 8.14-8.04 (m, 1H), 6.84-6.77 (m, 1H), 6.67 (s, 1H), 3.96-3.90 (m, 3H), 3.73-3.68 (m, 3H), 3.58 (s, 2H).

### Step B: Preparation of methyl 3-cyano-2-(2-methoxypyridin-4-yl)propanoate

To a solution of methyl 2-(2-methoxypyridin-4-yl)acetate (2, 35 g, 192 mmol, 1.0 eq.) in THF (500 mL) was added dropwise LDA (148 mL, 296 mmol, 2M, 1.5 eq.) at −65° C. under N₂. The reaction solution was stirred at −65° C. for 1 hr and 2-bromoacetonitrile (28.4 g, 239 mmol, 1.2 eq.) was added into the reaction mixture. After addition, the mixture was stirred at −65° C. for additional 1 hr. After completion, the reaction mixture was quenched with saturated NH₄Cl (aq.) solution (500 mL) and extracted with EtOAc (300 mL×3). The organic layers were combined and washed with brine (500 mL), dried over anhydrous Na₂SO₄ and concentrated under reduced pressure. The residue was purified by flash column chromatography on silica gel to afford methyl 3-cyano-2-(2-methoxypyridin-4-yl)propanoate (3.39 g, 177 mmol, 92%) as a yellow oil. LCMS (ESI): m/z=221.1 [M+H]⁺. ¹H NMR (400 MHz, CDCl₃) δ 8.18 (d, J=5.3 Hz, 1H), 6.79 (dd, J=5.3, 1.5 Hz, 1H), 6.66 (d, J=1.4 Hz, 1H), 3.94 (s, 3H), 3.88 (t, J=7.6 Hz, 1H), 3.75 (s, 3H), 3.06-2.97 (m, 1H), 2.87-2.77 (m, 1H).

### Step C: Preparation of 6-(2-methoxypyridin-4-yl)-4-azaspiro[2.4]heptan-5-one

To a solution of methyl 3-cyano-2-(2-methoxypyridin-4-yl)propanoate (3, 39 g, 177 mmol, 1.0 eq.) and Titanium tetraisopropanolate (23.3 g, 82 mmol, 0.5 eq.) in THF (800 mL) was added dropwise EtMgBr (134 mL, 402 mmol, 3M, 2.3 eq.) at 0° C. After addition, the reaction mixture was stirred at 0° C. for 1 hr. After completion, the reaction mixture was quenched with HCl aqueous solution (250 mL, 2N) and filtered, The filtrate was extracted with EtOAc (500 mL×3). The organic layers were combined, dried over anhydrous Na₂SO₄, and concentrated under reduced pressure. The residue was purified by flash column chromatography on silica gel to afford 6-(2-methoxypyridin-4-yl)-4-azaspiro[2.4]heptan-5-one (4, 12 g, 54.8 mmol, 31%) as a white solid. LCMS (ESI): m/z=219.2 [M+H]⁺. ¹H NMR (400 MHz, CDCl₃) δ 8.14 (d, J=5.3 Hz, 1H), 6.88 (dd, J=5.3, 1.4 Hz, 1H), 6.81-6.65 (m, 2H), 3.93 (s, 3H), 3.87-3.78 (m, 1H), 2.58-2.48 (m, 1H), 2.38-2.27 (m, 1H), 0.98-0.92 (m, 1H), 0.91-0.85 (m, 1H), 0.81-0.72 (m, 2H).

### Step D: Preparation of 6-(2-methoxypyridin-4-yl)-4-azaspiro[2.4]heptane

To a solution of 6-(2-methoxypyridin-4-yl)-4-azaspiro[2.4]heptan-5-one (4, 11 g, 50.2 mmol, 1.0 eq.) and methanidylidyneoxidanium tris(triphenylphosphane) hydrogen rhodium (2.31 g, 2.52 mmol, 0.05 eq.) in dioxane (200 mL) was added phenylsilane (33 g, 306 mmol, 6.0 eq.). The reaction solution was stirred at 100° C. for 12 hrs. After completion, the reaction mixture was cooled to 20° C. and poured into HCl aqueous solution (100 mL, 2N). The resulting solution was stirred at 20° C. for 1 hr and filtered. The filtrate was concentrated under reduced pressure to afford 6-(2-methoxypyridin-4-yl)-4-azaspiro[2.4]heptane (5, 11 g, 53.7 mmol, quant.) as a brown solid, which was used in next step directly without further purification. LCMS (ESI): m/z=205.2[M+H]⁺. ¹H NMR (400 MHz, CDCl₃) δ 8.08 (d, J=5.3 Hz, 1H), 6.79 (dd, J=5.3, 1.4 Hz, 1H), 6.64 (s, 1H), 3.93 (s, 3H), 3.49-3.36 (m, 2H), 3.03-2.91 (m, 1H), 2.19-2.15 (m, 1H), 1.97-1.87 (m, 1H), 0.85-0.80 (m, 2H), 0.66-0.58 (m, 2H).

### Step E: Preparation of 4-(4-azaspiro[2.4]heptan-6-yl)pyridin-2(1H)-one (HBr Salt)

A solution of 6-(2-methoxypyridin-4-yl)-4-azaspiro[2.4]heptane (5, 11 g, 53.7 mmol, 1.0 eq.) in a mixture of 30% HBr/AcOH (50 mL) and 30% HBr/H₂O (100 mL) was stirred at 100° C. for 12 hrs. After completion, the reaction mixture was concentrated under reduced pressure. The residue was neutralized carefully with NaHCO₃(aq.) until the pH was adjusted to pH=7. The mixture was concentrated under reduced pressure to afford crude 4-(4-azaspiro[2.4]heptan-6-yl)pyridin-2(1H)-one (6, 11 g, 57.6 mmol, quant.) as a yellow solid, which was used in next step directly without further purification. LCMS (ESI): m/z=191.2[M+H]⁺. ¹H NMR (400 MHz, DMSO-d₆) δ 11.48 (s, 1H), 7.37 (d, J=6.8 Hz, 1H), 6.29 (d, J=0.6 Hz, 1H), 6.22 (dd, J=6.8, 1.7 Hz, 1H), 3.71-3.62 (m, 1H), 3.56-3.41 (m, 1H), 3.26-3.19 (m, 1H), 2.24-2.03 (m, 2H), 1.21-1.02 (m, 2H), 0.94-0.78 (m, 2H).

### Step F. Preparation of tert-butyl 6-(2-oxo-1,2-dihydropyridin-4-yl)-4-azaspiro[2.4]heptane-4-carboxylate

To a mixture of 4-(4-azaspiro[2.4]heptan-6-yl)pyridin-2(1H)-one (6, 11 g, 57.9 mmol, 1.0 eq.) and TEA (15 mL, 108 mmol, 1.9 eq.) in THF (200 mL) was added Boc₂O (20 g, 91.7 mmol, 1.6 eq.). The resulting mixture was stirred at 20° C. for 12 hrs. After completion, the reaction mixture was concentrated under reduced pressure. The residue was purified by flash column chromatography on silica gel to afford tert-butyl 6-(2-oxo-1,2-dihydropyridin-4-yl)-4-azaspiro[2.4]heptane-4-carboxylate (7.4 g, 13.7 mmol, 24%) as a yellow solid. LCMS (ESI): m/z=291 [M+H]⁺.

### Step G: Preparation of tert-butyl 6-(1-methyl-2-oxo-1,2-dihydropyridin-4-yl)-4-azaspiro[2.4]heptane-4-carboxylate

To a solution of tert-butyl 6-(2-oxo-1,2-dihydropyridin-4-yl)-4-azaspiro[2.4]heptane-4-carboxylate (7, 4 g, 13.7 mmol, 1.0 eq.) and Cs₂CO₃ (14 g, 42.9 mmol, 3.1 eq.) in DMF (100 mL) was added MeI (2 mL, 32.1 mmol, 2.3 eq.). The resulting mixture was stirred at 20° C. for 12 hrs. After completion, the mixture was diluted with water (100 mL) and extracted with EtOAc (150 mL×3). The organic layers were combined and washed with brine (100 mL×2), dried over anhydrous Na₂SO₄, and concentrated under reduced pressure. The residue was purified by flash column chromatography on silica gel to afford tert-butyl 6-(1-methyl-2-oxo-1,2-dihydropyridin-4-yl)-4-azaspiro[2.4]heptane-4-carboxylate (8, 3.0 g, 9.84 mmol, 72%) as a yellow oil. LCMS (ESI) m/z=305 [M+H]⁺.

### Step H. Preparation of 1-methyl-4-(4-azaspiro[2.4]heptan-6-yl)pyridin-2(1H)-one

To a solution of tert-butyl 6-(1-methyl-2-oxo-1,2-dihydropyridin-4-yl)-4-azaspiro[2.4]heptane-4-carboxylate (8, 3.0 g, 9.84 mmol, 1.0 eq.) in DCM (30 mL) was added TFA (6 mL) and the resulting mixture was stirred at 20° C. for 1 hr. After completion, the mixture was concentrated under reduced pressure to give crude 1-methyl-4-(4-azaspiro[2.4]heptan-6-yl)pyridin-2(1H)-one (9, 2 g, 9.76 mmol, quant.) as a red oil, which was used in next step directly without further purification. LCMS (ESI): m/z=205 [M+H]⁺. tert-butyl 6-(1-methyl-2-oxo-1,2-dihydropyridin-4-yl)-4-azaspiro[2.4]heptane-4-carboxylate was prepared as described above to afford a racemic mixture. The individual stereoisomers were purified by chiral SFC conditions and absolute configuration was assigned as drawn below, based on the comparison of experimental vibrational circular dichroism (VCD) spectra with theoretical VCD spectra obtained from DFT calculations.

Preparative separation method for tert-butyl 6-(1-methyl-2-oxo-1,2-dihydropyridin-4-yl)-4-azaspiro[2.4]heptane-4-carboxylate:

Instrument: Waters Thar 80 preparative SFC; Column: ChiralPak AD, 250×21.2 mm I.D., 5 μm; Mobile phase: A for CO₂ and B for methanol (0.1% NH₃H₂O); Gradient: B 40%; Flow rate: 40 mL/min; Back pressure: 100 bar; Column temperature: 35° C.; Wavelength: 220 nm; Cycle-time: 10 min.

(S)-1-methyl-4-(4-azaspiro[2.4]heptan-6-yl)pyridin-2(1H)-one and (R)-1-methyl-4-(4-azaspiro[2.4]heptan-6-yl)pyridin-2(1H)-one were each prepared with the procedure described for racemic 1-methyl-4-(4-azaspiro[2.4]heptan-6-yl)pyridin-2(1H)-one utilizing the appropriate starting materials.

tert-butyl 6-(2-oxo-1,2-dihydropyridin-4-yl)-4-azaspiro[2.4]heptane-4-carboxylate was prepared as described above to afford a racemic mixture. The individual stereoisomers were purified by chiral SFC conditions and absolute configuration was assigned as drawn below. Assignment of absolute configuration was verified by independent synthesis of enantiopure tert-butyl (S)-6-(1-methyl-2-oxo-1,2-dihydropyridin-4-yl)-4-azaspiro[2.4]heptane-4-carboxylate via alkylation of tert-butyl (S)-6-(2-oxo-1,2-dihydropyridin-4-yl)-4-azaspiro[2.4]heptane-4-carboxylate, and of enantiopure tert-butyl (R)-6-(1-methyl-2-oxo-1,2-dihydropyridin-4-yl)-4-azaspiro[2.4]heptane-4-carboxylate via alkylation of tert-butyl (R)-6-(2-oxo-1,2-dihydropyridin-4-yl)-4-azaspiro[2.4]heptane-4-carboxylate, with the procedures described for racemic tert-butyl 6-(1-methyl-2-oxo-1,2-dihydropyridin-4-yl)-4-azaspiro[2.4]heptane-4-carboxylate.

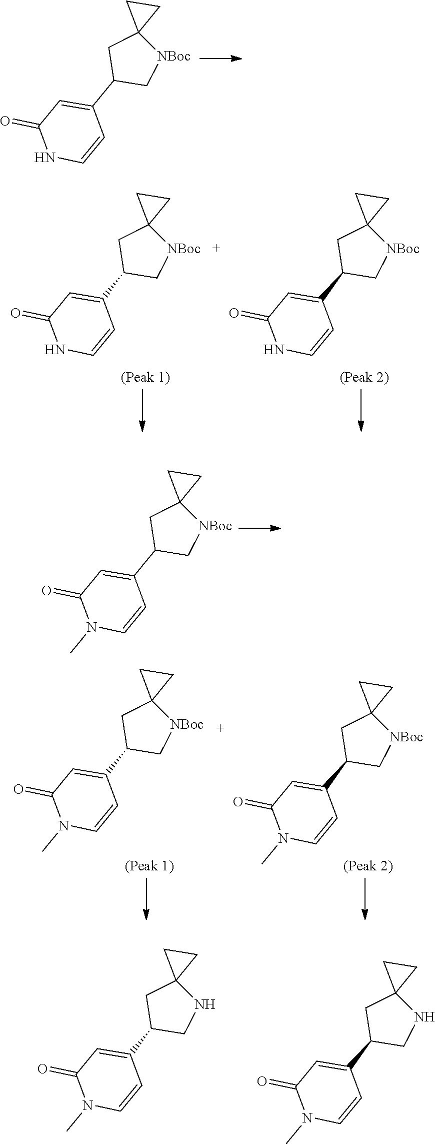

Preparative separation method for tert-butyl 6-(2-oxo-1,2-dihydropyridin-4-yl)-4-azaspiro[2.4]heptane-4-carboxylate:

Instrument: SHIMADZU PREP SOLUTION SFC; Column: ChiralPak AD, 250×30 mm I.D., 5 μm; Mobile phase: A for CO₂ and B for methanol (0.1% NH₃H₂O); Gradient: B 40%; Flow rate: 60 mL/min; Back pressure: 100 bar; Column temperature: 35° C.; Wavelength: 220 nm; Cycle-time: 7 min.

TABLE 24

| Name | Structure | LCMS |
|---|---|---|
| tert-butyl (S)-6-(1-methyl-2- oxo-1,2-dihydropyridin- 4-yl)-4-azaspiro[2.4]heptane- 4-carboxylate (Peak 1) | 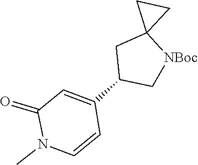 [SMILES: Cn1ccc([C@H]2CN(C(=O)OC(C)(C)C)C3(CC3)C2)cc1=O] | 305 [M + H]⁺ |
| tert-butyl (R)-6-(1-methyl-2- oxo-1,2-dihydropyridin- 4-yl)-4-azaspiro[2.4]heptane- 4-carboxylate (Peak 2) | 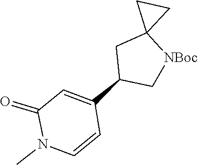 [SMILES: Cn1ccc([C@@H]2CN(C(=O)OC(C)(C)C)C3(CC3)C2)cc1=O] | 305 [M + H]⁺ |
| tert-butyl (S)-6-(2-oxo-1,2- dihydropyridin-4-yl)-4- azaspiro[2.4]heptane-4- carboxylate (Peak 1) | 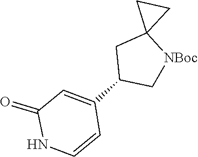 [SMILES: CC(C)(C)OC(=O)N1C[C@H](c2cc[nH]c(=O)c2)CC12CC2] | 291 [M + H]⁺ |
| tert-butyl (R)-6-(2-oxo-1,2- dihydropyridin-4-yl)-4- azaspiro[2.4]heptane-4- carboxylate (Peak 2) | 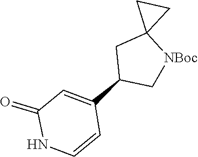 [SMILES: CC(C)(C)OC(=O)N1C[C@@H](c2cc[nH]c(=O)c2)CC12CC2] | 291 [M + H]⁺ |

(S)-4-(4-azaspiro[2.4]heptan-6-yl)pyridin-2(1H)-one and (R)-4-(4-azaspiro[2.4]heptan-6-yl)pyridin-2(1H)-one were each prepared with the procedure described for 1-methyl-4-(4-azaspiro[2.4]heptan-6-yl)pyridin-2(1H)-one utilizing the appropriate starting materials.

### Synthesis of 6-(pyridin-3-yl)-4,6-diazaspiro[2.4]heptane-5,7-dione

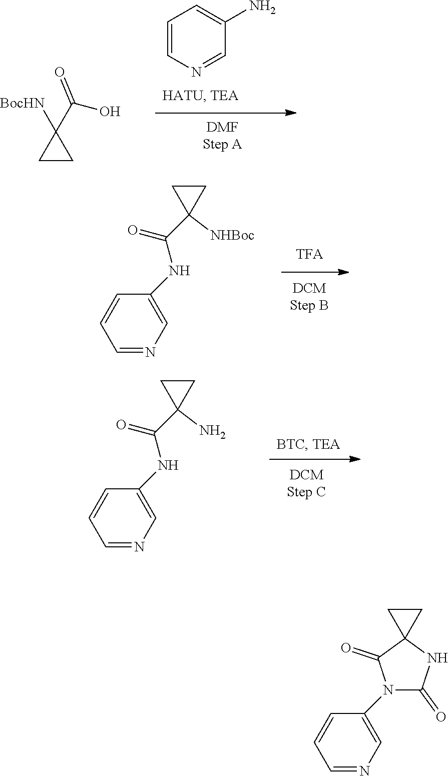

### Step A: Preparation of tert-butyl (1-(pyridin-3-ylcarbamoyl)cyclopropyl)carbamate

To a solution of 1-((tert-butoxycarbonyl)amino)cyclopropane-1-carboxylic acid (4 g, 19.8 mmol, 1 eq.) in DMF (50 mL) were added HATU (8.24 g, 21.7 mmol, 1.1 eq.), TEA (3.99 g, 39.6 mmol, 2 eq.) and pyridin-3-amine (1.86 g, 19.8 mmol, 1 eq.). The solution was stirred at room temperature for 2 hrs. After completion, the reaction mixture was concentrated under reduced pressure. The residue was purified by Biotage® C18 column chromatography to afford tert-butyl (1-(pyridin-3-ylcarbamoyl)cyclopropyl)carbamate (4.20 g, 15.1 mmol, 77%) as a yellow solid. LC-MS (ESI) m/z=278 [M+H]⁺.

### Step B: Preparation of 1-amino-N-(pyridin-3-yl)cyclopropane-1-carboxamide

To a solution of tert-butyl (1-(pyridin-3-ylcarbamoyl)cyclopropyl)carbamate (4 g, 14.4 mmol, 1 eq.) in DCM (20 mL) was added TFA (10 mL). The solution was stirred at room temperature for 2 hrs. After completion, the reaction mixture was concentrated under reduced pressure. The residue was cooled down in an ice bath, then neutralized carefully with NaHCO₃(aq.) until the pH was adjusted to pH=8-9. The resulting mixture was extracted with DCM (40 mL×3), and the combined organic layers were washed with brine (10 mL×2), dried over with anhydrous Na₂SO₄, then concentrated under reduced pressure to give crude 1-amino-N-(pyridin-3-yl)cyclopropane-1-carboxamide (2.5 g, quant.) as a yellow oil, which was used in next step directly without further purification. LCMS (ESI): m/z=178 [M+H]⁺.

### Step C: Preparation of 6-(pyridin-3-yl)-4,6-diazaspiro[2.4]heptane-5,7-dione

To a solution of 1-amino-N-(pyridin-3-yl)cyclopropane-1-carboxamide (2.2 g, 12.4 mmol, 1 eq.) and TEA (2.50 g, 24.8 mmol, 2 eq) in DCM (200 mL) was added BTC (1.46 g, 4.96 mmol, 0.4 eq.). The solution was stirred at room temperature for 1 hrs. After completion, the reaction mixture was concentrated under reduced pressure. The residue was purified by flash column chromatography on silica gel to afford 6-(pyridin-3-yl)-4,6-diazaspiro[2.4]heptane-5,7-dione (2.00 g, 9.84 mmol, 80% yield) as a white solid. LC-MS (ESI) m/z=204 [M+H]⁺.

### Synthesis of 5-chlorobenzo[d]isoxazole-3-carboxylic Acid

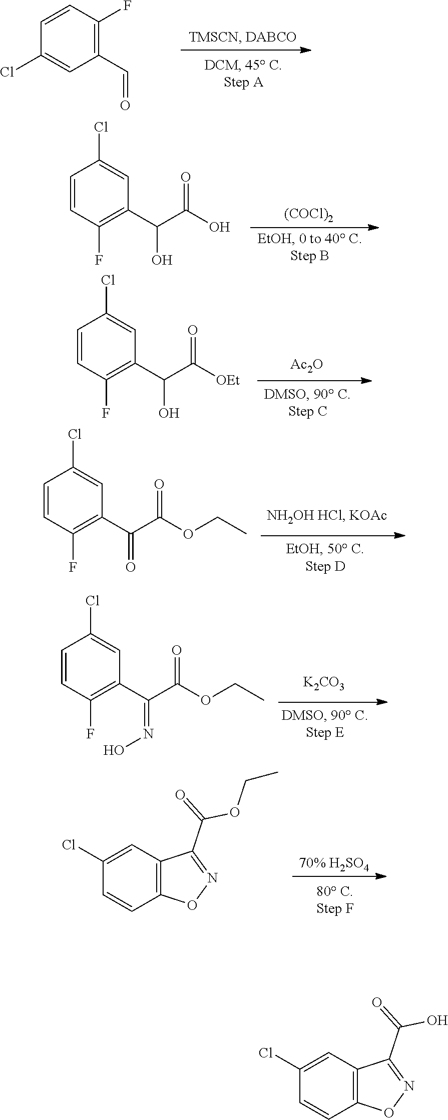

### Step A: Preparation of 2-(5-chloro-2-fluorophenyl)-2-hydroxyacetic Acid

To a solution of 5-chloro-2-fluorobenzaldehyde (12.4 g, 78.2 mmol, 1 eq.) and 1,4-Diazabicyclo[2.2.2]octane (613 mg, 5.47 mmol, 0.07 eq.) in DCM (40 mL) was added trimethylsilanecarbonitrile (8.29 g, 83.6 mmol, 1.07 eq.) at room temperature. After addition, the reaction mixture was heated at 45° C. for 30 min. The reaction mixture was cooled to room temperature, diluted with H₂O (100 mL) and extracted with DCM (100 mL×3). The organic layers were combined and washed with brine (100 mL), dried over anhydrous Na₂SO₄, and concentrated under reduced pressure to give 2-(5-chloro-2-fluorophenyl)-2-hydroxyacetic acid (14.3 g, quant.) as a white solid, which was used in next step directly without further purification. LCMS (ESI): m/z=203.0 [M−H]⁻.

### Step B: Preparation of ethyl 2-(5-chloro-2-fluorophenyl)-2-hydroxyacetate

To a solution of 2-(5-chloro-2-fluorophenyl)-2-hydroxyacetic acid (14.3 g, 69.8 mmol, 1 eq.) in EtOH (150 mL) was added (COCl)₂ (24.8 g, 209 mmol, 3.0 eq.) dropwise at 0° C. After addition, the reaction mixture was stirred at 40° C. for 2 hrs. After completion, the reaction mixture was concentrated under reduced pressure. The residue was dissolved in DCM (200 mL) and washed with water (50 mL), brine (50 mL), dried over anhydrous Na₂SO₄ and concentrated under reduced pressure to give ethyl 2-(5-chloro-2-fluorophenyl)-2-hydroxyacetate (15.0 g, quant.) as a colorless oil, which was used in next step directly without further purification. ¹H NMR (400 MHz, CDCl₃) δ 7.42-7.39 (m, 1H), 7.31-7.28 (m, 1H), 7.04 (t, J=9.1 Hz, 1H), 5.37 (s, 1H), 4.36-4.18 (m, 2H), 3.61 (s, 1H), 1.25 (t, J=7.1 Hz, 3H).

### Step C: Preparation of ethyl 2-(5-chloro-2-fluorophenyl)-2-oxoacetate

To a solution of ethyl 2-(5-chloro-2-fluorophenyl)-2-hydroxyacetate (15.0 g, 64.4 mmol, 1 eq.) in DMSO (30 mL) was added Ac₂O (8.08 g, 79.2 mmol, 1.23 eq.) dropwise at 90° C. After addition, the reaction mixture was stirred at 90° C. for 3 hrs. After completion, the reaction mixture was cooled to room temperature and diluted with water (100 mL) and extracted with DCM (100 mL×3). The organic layers were combined and washed with brine (100 mL), dried over anhydrous Na₂SO₄, and concentrated under reduced pressure to give ethyl 2-(5-chloro-2-fluorophenyl)-2-oxoacetate (12.2 g, 52.9 mmol, 82%) as a colorless oil, which was used in next step directly without further purification. ¹H NMR (400 MHz, CDCl₃) δ 7.88 (dd, J=5.9, 2.7 Hz, 1H), 7.64-7.54 (m, 1H), 7.15 (t, J=9.3 Hz, 1H), 4.44 (q, J=7.1 Hz, 2H), 1.41 (t, J=7.2 Hz, 3H).

### Step D: ethyl (E)-2-(5-chloro-2-fluorophenyl)-2-(hydroxyimino)acetate

To a solution of ethyl 2-(5-chloro-2-fluorophenyl)-2-oxoacetate (1.0 g, 4.33 mmol, 1 eq.) in EtOH (100 mL) were added hydroxylamine hydrochloride (450 mg, 6.49 mmol, 1.5 eq.) and KOAc (509 mg, 5.19 mmol, 1.2 eq.). The solution was stirred at 60° C. overnight. After completion, the reaction mixture was concentrated under reduced pressure. The residue was diluted with H₂O (10 mL) and extracted with EtOAc (10 mL×3). The organic layers were combined and washed with brine (10 mL), dried over anhydrous Na₂SO₄ and concentrated under reduced pressure to afford ethyl (E)-2-(5-chloro-2-fluorophenyl)-2-(hydroxyimino)acetate (1.0 g, quant.) as a yellow oil, which was used in next step directly without further purification. LCMS (ESI): m/z=246 [M+H]⁺.

### Step E: Preparation of ethyl 5-chlorobenzo[d]isoxazole-3-carboxylate

To a solution of ethyl (E)-2-(5-chloro-2-fluorophenyl)-2-(hydroxyimino)acetate (1.1 g, 4.47 mmol, 1 eq.) in DMSO (10 mL) was added K₂CO₃ (740 mg, 5.36 mmol, 1.2 eq.). The solution was stirred at 90° C. overnight. After completion, the solution was cooled to room temperature, diluted with H₂O (10 mL) and extracted with EtOAc (10 mL×3). The organic layers were combined and washed with brine (10 mL), dried over anhydrous Na₂SO₄ and concentrated under reduced pressure. The residue was purified by flash column chromatography on silica gel to afford ethyl 5-chlorobenzo[d]isoxazole-3-carboxylate (600 mg, 2.65 mmol, 60%) as a white solid. LCMS (ESI): m/z=226 [M+H]⁺.

### Step F. Preparation of 5-chlorobenzo[d]isoxazole-3-carboxylic Acid

A solution of ethyl 5-chloro-1,2-benzoxazole-3-carboxylate (300 mg, 1.32 mmol, 1 eq.) in H₂SO₄ (10 mL, 70 wt %, aq.) was heated to 80° C. and stirred at 80° C. for 2 hrs. After completion, the reaction mixture was cooled to 25° C. and poured into ice-water (10 mL). The suspension was filtered and the filter cake was dried under vacuum to afford 5-chlorobenzo[d]isoxazole-3-carboxylic acid (250 mg, 1.26 mmol, 96%) as a white solid, which was used in next step directly without further purification. LCMS (ESI): m/z=198 [M+H]⁺.

### Synthesis of 5-methylbenzo[d]isoxazole-3-carboxylic Acid

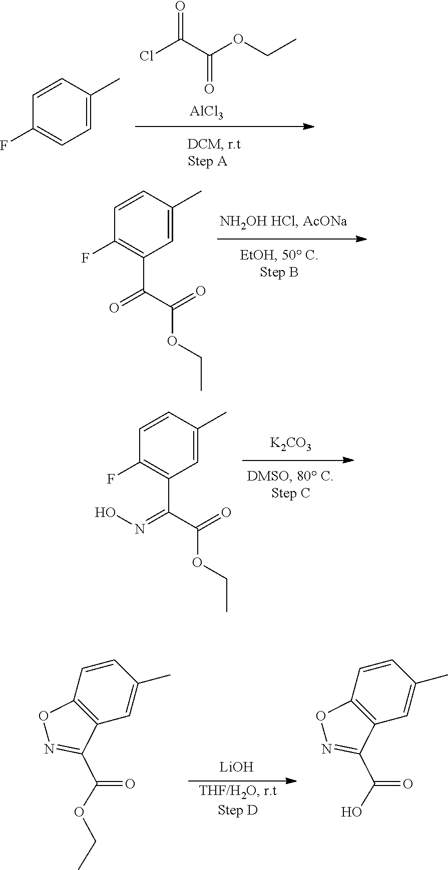

### Step A: Preparation of ethyl 2-(2-fluoro-5-methylphenyl)-2-oxoacetate

To a solution of AlCl₃ (17.2 g, 130 mmol, 1.2 eq.) in DCM (200 mL) were added ethyl 2-chloro-2-oxoacetate (15 g, 109 mmol, 1 eq.) and 1-fluoro-4-methylbenzene (12.0 g, 109 mmol, 1 eq.). The solution was stirred at room temperature for 2 hrs. After completion, the solution was poured into H₂O (200 mL), then extracted with DCM (100 mL×2). The combined organic layers was washed with brine (100 mL), dried over anhydrous Na₂SO₄, and concentrated under reduced pressure to afford ethyl 2-(2-fluoro-5-methylphenyl)-2-oxoacetate (15.0 g, quant.) as a yellow oil, which was used in next step directly without further purification.

### Step B: Preparation of ethyl (E)-2-(2-fluoro-5-methylphenyl)-2-(hydroxyimino)acetate

To a solution of ethyl 2-(2-fluoro-5-methylphenyl)-2-oxoacetate (15 g, 71.3 mmol, 1 eq.) in EtOH (200 mL) were added hydroxylamine hydrochloride (7.36 g, 106 mmol, 1.5 eq.) and AcONa (8.69 g, 106 mmol, 1.5 eq.). The solution was stirred at 50° C. overnight. After completion, the reaction mixture was concentrated under reduced pressure. The residue was diluted with H₂O (100 mL), extracted with EtOAc (100 mL×3). The organic layers were combined and washed with brine (100 mL), dried over anhydrous Na₂SO₄, and concentrated under reduced pressure. The residue was purified by flash column chromatography on silica gel to afford ethyl (E)-2-(2-fluoro-5-methylphenyl)-2-(hydroxyimino)acetate (15.0 g, 66.6 mmol, 94%) as a yellow oil.

### Step C: Preparation of ethyl 5-methylbenzo[d]isoxazole-3-carboxylate

To a solution of ethyl (E)-2-(2-fluoro-5-methylphenyl)-2-(hydroxyimino)acetate (15 g, 66.6 mmol, 1 eq.) in DMSO (30 mL) was added K₂CO₃ (18.3 g, 133 mmol, 2 eq.). The solution was stirred at 80° C. overnight. After completion, the solution was cooled to room temperature, diluted with H₂O (100 mL) and extracted with EtOAc (100 mL×3). The organic layers were combined and washed with brine (100 mL), dried over anhydrous Na₂SO₄, and concentrated under reduced pressure to afford ethyl 5-methylbenzo[d]isoxazole-3-carboxylate (7.8 g, 38 mmol, 57%) as a yellow solid, which was used in next step without further purification.

### Step D: Preparation of 5-methylbenzo[d]isoxazole-3-carboxylic Acid

To a solution of ethyl 5-methylbenzo[d]isoxazole-3-carboxylate (7.8 g, 38.0 mmol, 1 eq.) in THF (30 mL) and H₂O (30 mL) was added lithium(1+) hydrate hydroxide (4.74 g, 113 mmol, 3 eq.). The solution was stirred at room temperature for 2 hrs. After completion, the reaction mixture was quenched with HCl (1N, aq.) until the pH was adjusted to pH=5, then extracted with EtOAc (100 mL×2). The organic layers were combined and washed with brine (100 mL), dried over anhydrous Na₂SO₄, and concentrated under reduced pressure to afford 5-methylbenzo[d]isoxazole-3-carboxylic acid (6.0 g, 33.8 mmol, 89%) as a white solid, which was used in next step directly without further purification. LCMS (ESI): m/z=178 [M+H]⁺.

### Synthesis of 8-chloro-2-methylquinoline-6-carboxylic Acid

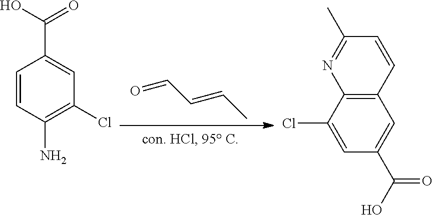

A solution of 4-amino-3-chlorobenzoic acid (3.7 g, 21.5 mmol, 1.0 eq.) and (E)-but-2-enal (3.01 g, 43.0 mmol, 2.0 eq.) in conc. HCl (10 mL) was stirred at 95° C. for 2 hrs under N₂. After completion, the reaction was concentrated under reduced pressure. The residue was purified by flash chromatography on C18 (10-40% ACN in water) to afford 8-chloro-2-methylquinoline-6-carboxylic acid (2.80 g, 12.6 mmol, 59%) as a brown solid. LCMS (ESI): m/z=222 [M+H]⁺. ¹H NMR (400 MHz, DMSO) δ 13.47 (s, 1H), 8.60 (d, J=1.4 Hz, 1H), 8.53 (d, J=8.5 Hz, 1H), 8.25 (d, J=1.6 Hz, 1H), 7.63 (d, J=8.4 Hz, 1H), 2.75 (s, 3H).

### Preparation of Intermediate: methyl (4S,7S,9aR)-4-((tert-butoxycarbonyl)amino)-5-oxo-2-(pyridin-2-yl)octahydro-1H-pyrrolo[1,2-a][1,4]diazepine-7-carboxylate

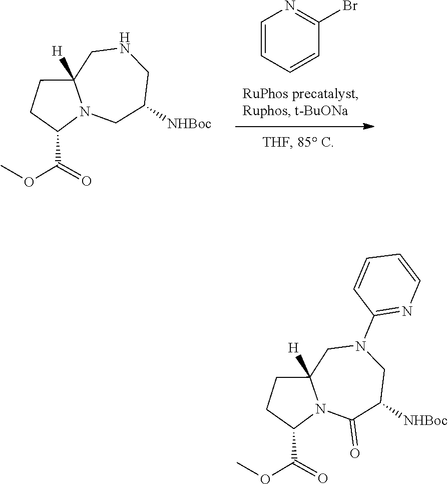 [SMILES: COC(=O)[C@@H]1CC[C@@H]2CNC[C@H](NC(=O)OC(C)(C)C)CN21 ; Brc1ccccn1 ; COC(=O)[C@@H]1CC[C@@H]2CN(c3ccccn3)C[C@H](NC(=O)OC(C)(C)C)C(=O)N21]

To a solution of methyl (4S,7S,9aR)-4-((tert-butoxycarbonyl)amino)-5-oxooctahydro-1H-pyrrolo[1,2-a][1,4]diazepine-7-carboxylate (618 mg, 1.89 mmol, 1 eq.) and 2-bromopyridine (300 mg, 1.89 mmol, 1.0 eq.) in THF (10 ml) were added RuPhos precatalyst (154 mg, 0.19 mmol, 0.1 eq.), RuPhos (88 mg, 0.19 mmol, 0.1 eq.) and t-BuONa (544 mg, 5.67 mmol, 3 eq.). The reaction mixture was stirred at 80° C. for 16 hrs under N₂ atmosphere. After completion, the reaction mixture was diluted with H₂O (10 mL), extracted with EtOAc (10 mL×3). The organic layers were combined and washed with brine (10 mL), dried over anhydrous Na₂SO₄, and concentrated under reduced pressure to give (5S,8S,10aR)-5-((tert-butoxycarbonyl)amino)-3-(isoquinolin-3-yl)-6-oxodecahydropyrrolo[1,2-a][1,5]diazocine-8-carboxylic acid (300 mg, quant.) as a yellow oil, used without further purification. LCMS (ESI): m/z=405 [M+H]⁺.

### Phosphonate Acids Data Tables

The following compounds in Table 25 were prepared according to the representative procedure described above for the synthesis of S,S′-(((((7-(((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate) (1) and utilizing the appropriate starting materials and modifications.

TABLE 25

| Compound | Name | Structure | LCMS | NMR |
|---|---|---|---|---|
| 354 | ((7- (((5S,8S,10a R)-3-acetyl- 8-(6-(1- methyl-2- oxo-1,2- dihydropyridin- 4-yl)-4- azaspiro[2.4] heptane-4- carbonyl)-6- oxodeca- hydropyrrolo [1,2-a][1,5] diazocin-5- yl)carbamoyl) naphthalen- 2- yl) fluoromethyl) phosphonic acid | 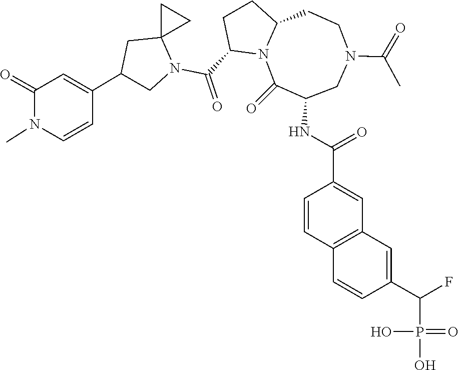 [SMILES: CC(=O)N1CC[C@H]2CC[C@@H](C(=O)N3CC(c4ccn(C)c(=O)c4)CC34CC4)N2C(=O)[C@@H](NC(=O)c2ccc3ccc(C(F)P(=O)(O)O)cc3c2)C1] | 722.1 [M + H]⁺ | ¹H NMR (400 MHZ, DMSO) δ 8.93-8.48 (m, 2H), 8.12-7.93 (m, 4H), 7.72-7.55 (m, 2H), 6.39-6.30 (m, 1H), 6.27-6.19 (m, 1H), 5.97-5.81 (m, 1H), 5.06 4.91 (m, 1H), 4.60-4.51 (m, 1H), 4.36-4.21 (m, 2H), 4.11 4.01 (m, 1H), 3.85-3.79 (m, 1H), 3.74 3.56 (m, 4H), 3.38-3.36 (m, 3H), 2.35 2.25 (m, 1H), 2.21-2.10 (m, 3H), 2.10-2.07 (m, 2H), 2.06 1.91 (m, 3H), 1.86-1.65 (m, 3H), 1.62-1.51 (m, 1H), 0.55-0.37 (m, 2H) |
| 355 | ((7- (((5S,8S,10a R)-3-acetyl- 6-oxo-8-(3- (pyridin-3- yl)pyrrolidine- 1-carbonyl) decahydro- pyrrolo [1,2-a][1,5] diazocin-5- yl)carbamoyl) naphthalen- 2-yl) fluoromethyl) phosphonic acid | 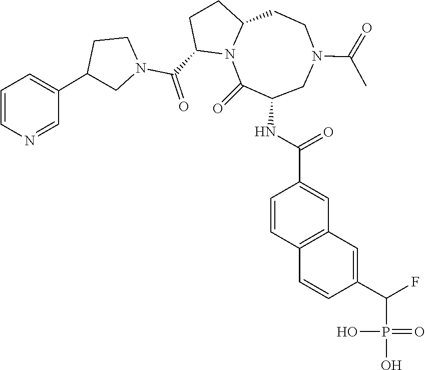 [SMILES: CC(=O)N1CC[C@H]2CC[C@@H](C(=O)N3CCC(c4cccnc4)C3)N2C(=O)[C@@H](NC(=O)c2ccc3ccc(C(F)P(=O)(O)O)cc3c2)C1] | 666.3 [M+H]+ | ¹H NMR (400 MHZ, DMSO-d₆) δ 8.85- 7.31 (m, 11H), 5.98- 5.78 (m, 1H), 5.08- 4.89 (m, 1H), 4.71 4.58 (m, 1H), 4.38- 4.22 (m, 2H), 4.09 3.82 (m, 3H), 3.80 3.73 (m, 1H), 3.64 3.54 (m, 1H), 3.49 3.27 (m, 2H), 2.43- 1.67 (m, 11H) |
| 356 | ((7- (((5S,8S,10a R)-3-acetyl- 8-(6-(1- methyl-2- oxo-1,2- dihydropyrid in- 4-yl)-4- azaspiro[2.4] heptane-4- carbonyl)-6- oxodeca- hydropyrrolo [1,2-a][1,5] diazocin-5- yl)carbamoyl) naphthalen- 2- yl)methyl) phosphonic acid | 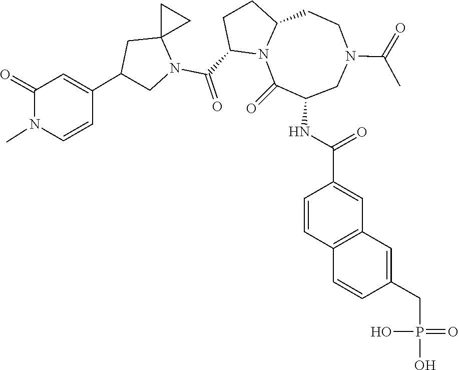 [SMILES: CC(=O)N1CC[C@H]2CC[C@@H](C(=O)N3CC(c4ccn(C)c(=O)c4)CC34CC4)N2C(=O)[C@@H](NC(=O)c2ccc3ccc(CP(=O)(O)O)cc3c2)C1] | 704.4 [M + H]⁺ | ¹H NMR (400 MHZ, CDCl₃) δ 8.62-8.13 (m, 1H), 8.11-7.84 (m, 1H), 7.77-7.21 (m, 4H), 7.16-6.91 (m, 1H), 6.35-5.64 (m, 2H), 4.88-4.01 (m, 9H), 3.41-2.93 (m, 6H), 2.35-1.54 (m, 10H), 1.37-1.07 (m, 3H), 0.86-0.10 (m, 2H) |

The following compounds in Table 26 were prepared according to the representative procedure described above for the synthesis of (difluoro(2-(((5S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonic acid (3) utilizing the appropriate starting materials and modifications.

TABLE 26

| Com- | | | | |
|---|---|---|---|---|
| pound | Name | Structure | LCMS | NMR |
| 357 | (fluoro(7-(((5S,8S,10aR)- 3-(imidazo[1,5-a]pyridine-1- carbonyl)-8-(morpholine- 4-carbonyl)-6-oxodecahydro- pyrrolo[1,2-a][1,5]diazocin- 5-yl)carbamoyl)naphthalen- 2-yl)methyl)phosphonic acid | 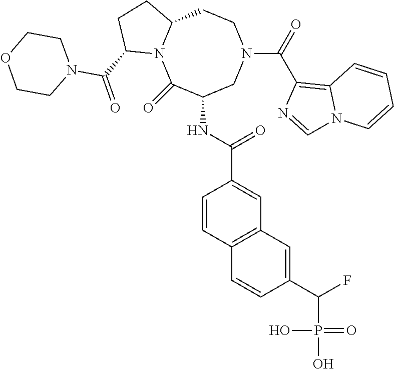 [SMILES: O=C(N[C@H]1CN(C(=O)c2ncn3ccccc23)CC[C@H]2CC[C@@H](C(=O)N3CCOCC3)N2C1=O)c1ccc2ccc(C(F)P(=O)(O)O)cc2c1] | 707.3 [M + H]⁺ | ¹H NMR (400 MHz, DMSO) δ 8.95-8.70 (m, 1H), 8.64-8.46 (m, 2H), 8.44-8.34 (m, 1H), 8.26-7.94 (m, 5H), 7.71-7.65 (m, 1H), 7.17-7.07 (m, 1H), 6.93-6.83 (m, 1H), 6.03-5.82 (m, 1H), 5.44-4.91 (m, 2H), 4.88-4.11 (m, 3H), 3.94-3.10 (m, 10H), 2.39-1.53 (m, 6H). (TFA salt) |
| 358 | (fluoro(7-(((5S,8S,10aR)- 8-(morpholine-4-carbonyl)- 6-oxo-3-(4,5,6,7-tetra- hydrobenzo[d]isoxazole- 3-carbonyl)decahydropyrrolo [1,2-a][1,5]diazocin-5- yl)carbamoyl)naphthalen-2- yl)methyl)phosphonic acid | 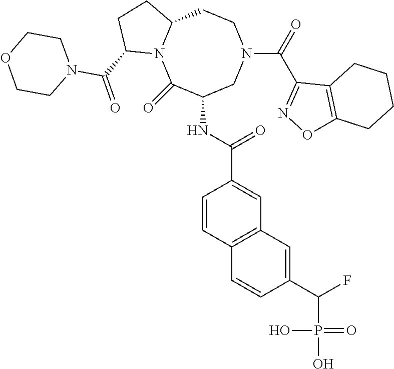 [SMILES: O=C(N[C@H]1CN(C(=O)c2noc3c2CCCC3)CC[C@H]2CC[C@@H](C(=O)N3CCOCC3)N2C1=O)c1ccc2ccc(C(F)P(=O)(O)O)cc2c1] | 712.2 [M + H]⁺ | ¹H NMR (400 MHz, DMSO) δ 8.91-8.69 (m, 1H), 8.61-8.46 (m, 1H), 8.09-7.98 (m, 3H), 7.98-7.89 (m, 1H), 7.71-7.64 (m, 1H), 6.01-5.83 (m, 1H), 5.17-4.89 (m, 2H), 4.85-4.78 (m, 1H), 4.42-4.22 (m, 2H), 4.04-3.86 (m, 2H), 3.83-3.63 (m, 2H), 3.63-3.59 (m, 1H), 3.58-3.49 (m, 5H), 3.46-3.10 (m, 2H), 2.79-2.62 (m, 2H), 2.34-2.21 (m, 1H), 2.19-2.03 (m, 1H), 1.97 (s, 1H), 1.88-1.60 (m, 6H), 1.28-1.23 (m, 3H) |
| 359 | (fluoro(7-(((5S,8S,10aR)-8- (morpholine-4-carbonyl)- 6-oxo-3-(pyrazolo[1,5-a] pyrimidine-2-carbonyl) decahydropyrrolo[1,2-a] [1,5]diazocin-5-yl) carbamoyl)naphthalen-2- yl)methyl)phosphonic acid | 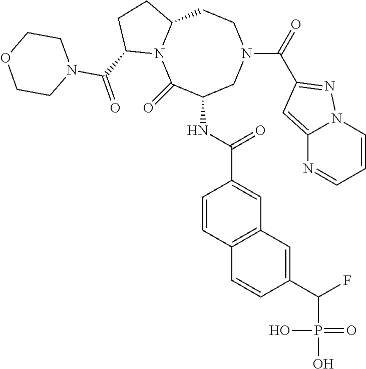 [SMILES: O=C(N[C@H]1CN(C(=O)c2cc3ncccn3n2)CC[C@H]2CC[C@@H](C(=O)N3CCOCC3)N2C1=O)c1ccc2ccc(C(F)P(=O)(O)O)cc2c1] | 708.2 [M + H]⁺ | ¹H NMR (400 MHz, DMSO) δ 9.21-8.87 (m, 1H), 8.68-8.61 (m, 1H), 8.61-8.56 (m, 1H), 8.44 (s, 1H), 8.13-7.94 (m, 3H), 7.90 (d, J = 8.6 Hz, 1H), 7.67 (dd, J = 14.5, 8.6 Hz, 1H), 7.23- 7.06 (m, 1H), 6.95 (s, 1H), 6.03-5.80 (m, 1H), 5.43-5.29 (m, 1H), 5.14-4.77 (m, 2H), 4.52-4.26 (m, 2H), 3.99-3.80 (m, 3H), 3.72-3.60 (m, 3H), 3.55-3.39 (m, 4H), 2.31-2.19 (m, 1H), 2.19-1.78 (m, 4H), 1.75-1.42 (m, 1H) |
| 360 | ((7-(((5S,8S,10aR)-3-(6,7- dihydro-4H-pyrano[3,4-d] isoxazole-3-carbonyl)-8- (morpholine-4-carbonyl)- 6-oxodecahydropyrrolo [1,2-a][1,5]diazocin-5-yl) carbamoyl)naphthalen-2- yl)fluoromethyl) phosphonic acid | 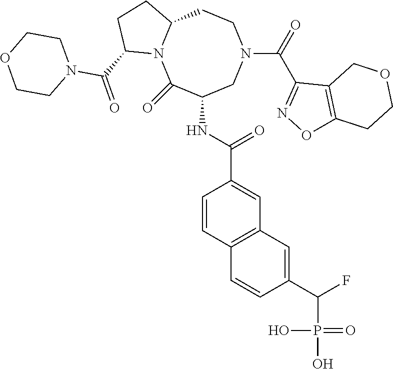 [SMILES: O=C(N[C@H]1CN(C(=O)c2noc3c2COCC3)CC[C@H]2CC[C@@H](C(=O)N3CCOCC3)N2C1=O)c1ccc2ccc(C(F)P(=O)(O)O)cc2c1] | 714.4 [M + H]⁺ | ¹H NMR (400 MHz, DMSO) δ 8.91-8.78 (m, 1H), 8.59-8.49 (m, 1H), 8.05-7.93 (m, 4H), 7.71-7.61 (m, 1H), 5.96-5.74 (m, 1H), 5.29-5.06 (m, 1H), 4.84-4.71 (m, 2H), 4.66-4.55 (m, 1H), 4.45-4.20 (m, 2H), 4.13-4.00 (m, 2H), 3.97-3.74 (m, 5H), 3.65-3.56 (m, 3H), 3.15-3.11 (m, 1H), 2.92-2.83 (m, 2H), 2.35-2.17 (m, 2H), 2.14-2.04 (m, 2H), 1.90-1.63 (m, 4H) |
| 361 | (fluoro(7-(((5S,8S,10aR)- 8-(morpholine-4- carbonyl)-6-oxo-3- (propylsulfonyl) decahydropyrrolo[1,2-a] [1,5]diazocin-5-yl) carbamoyl)naphthalen-2- yl)methyl)phosphonic acid | 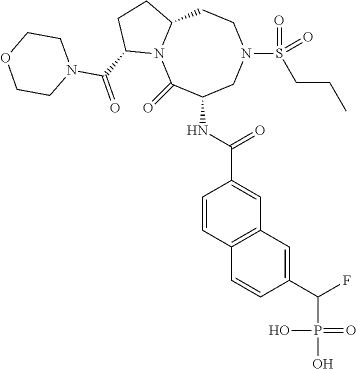 [SMILES: CCCS(=O)(=O)N1CC[C@H]2CC[C@@H](C(=O)N3CCOCC3)N2C(=O)[C@@H](NC(=O)c2ccc3ccc(C(F)P(=O)(O)O)cc3c2)C1] | 667.2 [M − H]⁻ | ¹H NMR (400 MHz, DMSO) δ 8.72-8.39 (m, 2H), 8.14-7.46 (m, 5H), 5.81-5.62 (m, 1H), 4.99-4.75 (m, 2H), 4.51-4.40 (m, 1H), 3.87-3.81 (m, 1H), 3.72-3.63 (m, 2H), 3.59-3.47 (m, 7H), 3.41-3.35 (m, 1H), 3.27-3.10 (m, 3H), 2.39-2.25 (m, 1H), 2.17-2.00 (m, 1H), 1.90-1.62 (m, 6H), 1.04-0.95 (m, 3H) |
| 362 | (fluoro(7-(((5S,8S,10aR)-3- (5-methoxybenzo[d] isoxazole-3-carbonyl)-8- (morpholine-4-carbonyl)- 6-oxodecahydropyrrolo [1,2-a][1,5]diazocin-5- yl)carbamoyl)naphthalen- 2-yl)methyl)phosphonic acid | 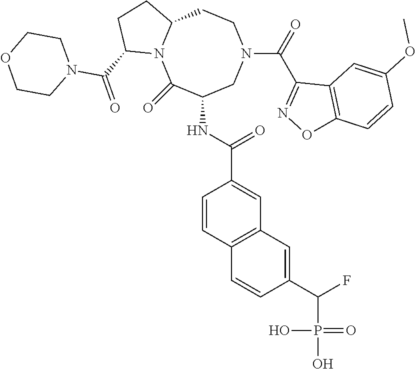 [SMILES: COc1ccc2onc(C(=O)N3CC[C@H]4CC[C@@H](C(=O)N5CCOCC5)N4C(=O)[C@@H](NC(=O)c4ccc5ccc(C(F)P(=O)(O)O)cc5c4)C3)c2c1] | 738.3 [M + H]⁺ | ¹H NMR (400 MHz, DMSO) δ 8.98-8.71 (m, 1H), 8.62-8.43 (m, 1H), 8.11-7.87 (m, 4H), 7.79-7.59 (m, 2H), 7.58-7.43 (m, 1H), 7.37-7.25 (m, 1H), 5.98-5.75 (m, 1H), 5.31-5.15 (m, 1H), 4.85-4.79 (m, 1H), 4.54-4.40 (m, 1H), 4.12-4.00 (m, 3H), 3.84-3.82 (m, 3H), 3.57-3.51 (m, 5H), 3.50-3.42 (m, 4H), 2.35-1.76 (m, 5H), 1.75-1.54 (m, 1H) |
| 363 | (fluoro(7-(((5S,8S,10aR)-3- (6-methoxybenzoyl[d] isoxazole-3-carbonyl)-8- (morpholine-4-carbonyl)- 6-oxodecahydropyrrolo [1,2-a][1,5]diazocin-5- yl)carbamoyl) naphthalen-2-yl) methyl)phosphonic acid | 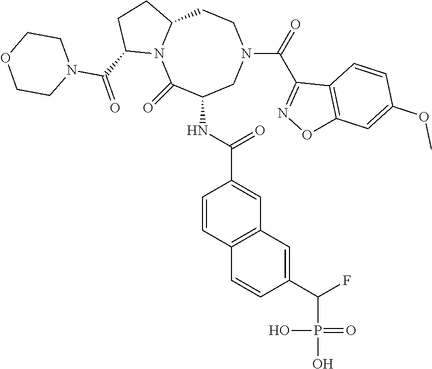 [SMILES: COc1ccc2c(C(=O)N3CC[C@H]4CC[C@@H](C(=O)N5CCOCC5)N4C(=O)[C@@H](NC(=O)c4ccc5ccc(C(F)P(=O)(O)O)cc5c4)C3)noc2c1] | 736.2 [M − H]⁻ | ¹H NMR (400 MHz, DMSO) δ 8.96-8.70 (m, 1H), 8.62-8.41 (m, 1H), 8.09-7.89 (m, 5H), 7.72-7.60 (m, 1H), 7.43-7.29 (m, 1H), 7.11-6.94 (m, 1H), 5.98-5.80 (m, 1H), 5.30-5.12 (m, 1H), 4.86-4.74 (m, 1H), 4.53-4.41 (m, 1H), 4.31-4.10 (m, 1H), 4.10-3.90 (m, 3H), 3.90-3.86 (m, 3H), 3.84-3.70 (m, 1H), 3.62-3.44 (m, 7H), 2.34-2.16 (m, 2H), 2.16-1.96 (m, 1H), 1.96-1.84 (m, 1H), 1.84-1.56 (m, 2H) |
| 364 | (fluoro(7-(((5S,8S,10aR)- 8-(morpholine-4-carbonyl)- 6-oxo-3-(5,6,7,8- tetrahydroimidazo[1,5- a]pyridine-1-carbonyl) decahydropyrrolo[1,2- a][1,5]diazocin-5-yl) carbamoyl)naphthalen- 2-yl)methyl)phosphonic acid | 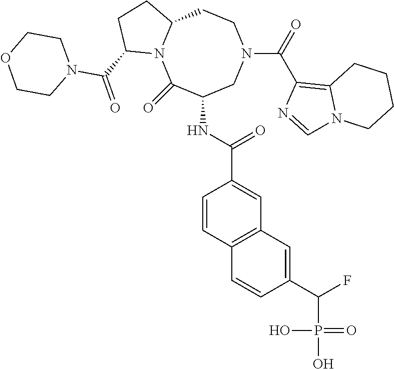 [SMILES: O=C(N[C@H]1CN(C(=O)c2ncn3c2CCCC3)CC[C@H]2CC[C@@H](C(=O)N3CCOCC3)N2C1=O)c1ccc2ccc(C(F)P(=O)(O)O)cc2c1] | 711.3 [M + H]⁺ | ¹H NMR (400 MHz, DMSO) δ 8.88-8.64 (m, 1H), 8.58-8.44 (m, 1H), 8.18-7.84 (m, 4H), 7.69-7.42 (m, 2H), 5.75-5.51 (m, 1H), 5.21-4.87 (m, 2H), 4.85-4.75 (m, 1H), 4.46-4.29 (m, 1H), 4.30-4.22 (m, 1H), 3.58-3.37 (m, 9H), 3.20-2.88 (m, 3H), 2.39-1.54 (m, 11H) (TFA salt) |
| 365 | ((7-(((5S,8S,10aR)-3-(5- chlorobenzo[d]isoxazole- 3-carbonyl)-8-(morpholine- 4-carbonyl)-6-oxodecahydro- pyrrolo[1,2-a][1,5]diazocin- 5-yl)carbamoyl)naphthalen- 2-yl)fluoromethyl) phosphonic acid | 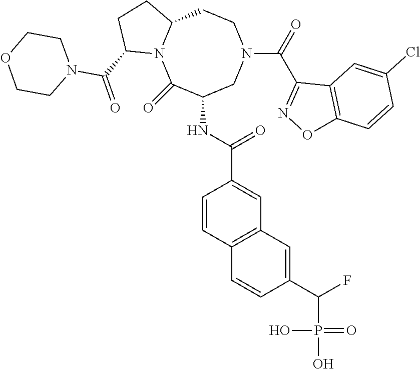 [SMILES: O=C(N[C@H]1CN(C(=O)c2noc3ccc(Cl)cc23)CC[C@H]2CC[C@@H](C(=O)N3CCOCC3)N2C1=O)c1ccc2ccc(C(F)P(=O)(O)O)cc2c1] | 742.2 [M + H]⁺ | ¹H NMR (400 MHz, DMSO) δ 9.02-8.76 (m, 1H), 8.64-8.43 (m, 1H), 8.34-8.21 (m, 1H), 8.09-7.96 (m, 3H), 7.96-7.86 (m, 2H), 7.80-7.61 (m, 2H), 6.04-5.76 (m, 1H), 5.42-5.24 (m, 1H), 4.90-4.70 (m, 1H), 4.58-4.46 (m, 1H), 4.21-4.14 (m, 3H), 3.79-3.70 (m, 1H), 3.65-3.26 (m, 8H), 2.48-2.35 (m, 1H), 2.30-2.01 (m, 2H), 1.92-1.49 (m, 3H) |

The following compounds in Table 27 were prepared according to the representative procedure described above for the synthesis of ((7-(((S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamoyl)naphthalen-2-yl)difluoromethyl)phosphonic acid (4) and utilizing the appropriate starting materials and modifications.

TABLE 27

| Com- | | | | |
|---|---|---|---|---|
| pound | Name | Structure | LCMS | NMR |
| 366 | ((7-(((5S,8S,10aR)-3-(8- chloro-2-methyl- quinoline-6-carbonyl)-8- (morpholine-4-carbonyl)- 6-oxodecahydropyrrolo [1,2-a][1,5]diazocin-5- yl)carbamoyl)naphthalen- 2-yl)fluoromethyl) phosphonic acid | 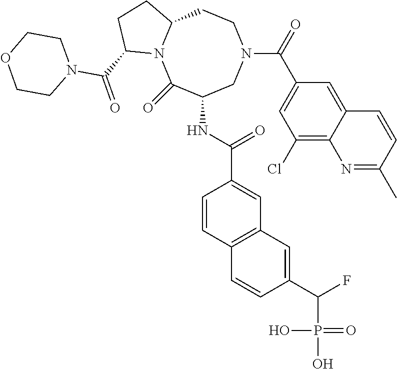 [SMILES: Cc1ccc2cc(C(=O)N3CC[C@H]4CC[C@@H](C(=O)N5CCOCC5)N4C(=O)[C@@H](NC(=O)c4ccc5ccc(C(F)P(=O)(O)O)cc5c4)C3)cc(Cl)c2n1] | 766.4 [M + H]⁺ | ¹H NMR (400 MHz, DMSO-d₆) δ 8.71- 8.58 (m, 3H), 8.44- 8.23 (m, 2H), 8.16- 7.83 (m, 6H), 7.67- 7.51 (m, 2H), 5.89- 5.68 (m, 1H), 5.44- 5.29 (m, 1H), 4.91- 4.76 (m, 1H), 4.63- 4.48 (m, 1H), 4.21- 4.03 (m, 1H), 3.89- 3.73 (m, 1H), 3.70- 3.49 (m, 10H), 2.72 (s, 3H), 2.42-2.11 (m, 3H), 1.89-1.67 (m, 3H) |
| 367 | (difluoro(7-(((5S,8S, 10aR)-3-(5-methyl- 1H-indazole-3-carbonyl)- 6-oxo-8-(6-(2-oxo-1,2- dihydropyridin-4- yl)-4-azaspiro[2.4] heptane-4-carbonyl) decahydropyrrolo[1,2- a][1,5]diazocin-5- yl)carbamoyl) naphthalen-2-yl)methyl) phosphonic acid | 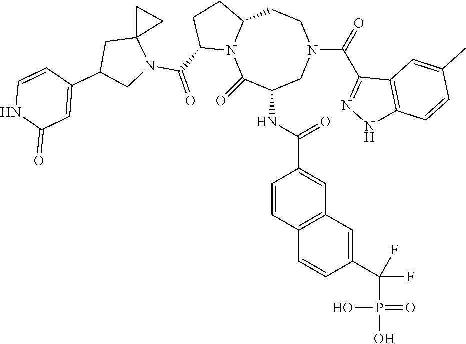 [SMILES: Cc1ccc2[nH]nc(C(=O)N3CC[C@H]4CC[C@@H](C(=O)N5CC(c6cc[nH]c(=O)c6)CC56CC6)N4C(=O)[C@@H](NC(=O)c4ccc5ccc(C(F)(F)P(=O)(O)O)cc5c4)C3)c2c1] | 842.1 [M + H]⁺ | ¹H NMR (400 MHz, DMSO-d₆) δ 13.57- 13.28 (m, 1H), 9.11- 8.74 (m, 1H), 8.66 (d, J = 25.3 Hz, 1H), 8.32- 8.13 (m, 1H), 8.10- 7.99 (m, 3H), 7.89 (d, J = 49.5 Hz, 1H), 7.78- 7.64 (m, 1H), 7.57- 7.34 (m, 1H), 7.34- 7.15 (m, 2H), 6.32- 6.02 (m, 2H), 5.26- 5.10 (m, 1H), 4.76- 4.44 (m, 2H), 4.39- 4.17 (m, 2H), 4.14- 3.98 (m, 1H), 3.88- 3.66 (m, 2H), 3.37- 3.25 (m, 2H), 2.42 (s, 3H), 2.24-1.96 (m, 4H), 1.96-1.41 (m, 6H), 0.55-0.24 (m, 2H) |
| 368 | (fluoro(7-(((5S,8S,10aR)- 3-(5-methylbenzo[d] isoxazole-3-carbonyl)- 8-(4-(methylsulfonyl) piperazine-1-carbonyl)- 6-oxodecahydropyrrolo [1,2-a][1,5]diazocin-5- yl)carbamoyl)naphthalen- 2-yl)methyl)phosphonic acid | 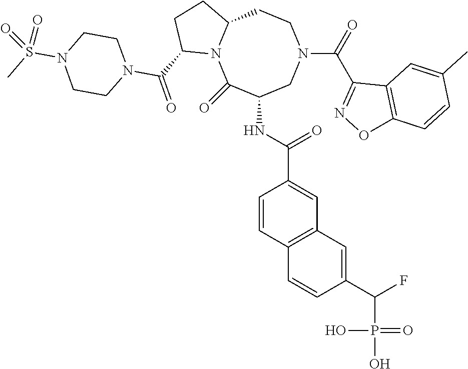 [SMILES: Cc1ccc2onc(C(=O)N3CC[C@H]4CC[C@@H](C(=O)N5CCN(S(C)(=O)=O)CC5)N4C(=O)[C@@H](NC(=O)c4ccc5ccc(C(F)P(=O)(O)O)cc5c4)C3)c2c1] | 799.2 [M + H]⁺ | ¹H NMR (400 MHz, DMSO-d₆) δ 8.98- 8.72 (m, 1H), 8.65- 8.44 (m, 1H), 8.13- 7.87 (m, 5H), 7.77- 7.62 (m, 2H), 7.59- 7.49 (m, 1H), 6.03- 5.81 (m, 1H), 5.32- 5.17 (m, 1H), 4.88- 4.80 (m, 1H), 4.52- 4.48 (m, 1H), 4.23- 4.21 (m, 1H), 4.08- 3.89 (m, 6H), 3.68- 3.60 (m, 2H), 3.51- 3.35 (m, 1H), 3.14- 3.03 (m, 2H), 2.92- 2.83 (m, 3H), 2.48- 2.42 (m, 3H), 2.36- 1.54 (m, 6H) |
| 369 | ((7-(((5S,8S,10aR)-8- (4-(benzo[d]isothiazol- 3-yl)piperazine-1- carbonyl)-3-(5- methylbenzo[d] isoxazole-3-carbonyl)-6- oxodecahydropyrrolo [1,2-a][1,5]diazocin-5- yl)carbamoyl) naphthalen-2-yl) fluoromethyl) phosphonic acid | 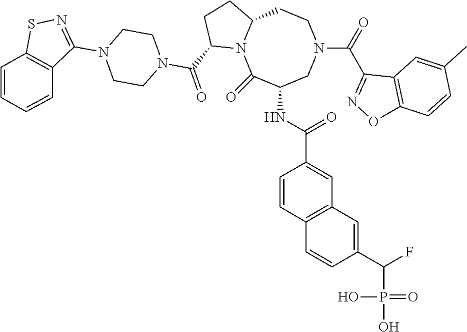 [SMILES: Cc1ccc2onc(C(=O)N3CC[C@H]4CC[C@@H](C(=O)N5CCN(c6nsc7ccccc67)CC5)N4C(=O)[C@@H](NC(=O)c4ccc5ccc(C(F)P(=O)(O)O)cc5c4)C3)c2c1] | 854.3 [M + H]⁺ | ¹H NMR (400 MHz, DMSO-d₆) δ 9.01- 8.72 (m, 1H), 8.65- 8.44 (m, 1H), 8.14- 7.87 (m, 7H), 7.76- 7.62 (m, 2H), 7.59- 7.50 (m, 2H), 7.47- 7.41 (m, 1H), 5.98- 5.84 (m, 1H), 5.36- 5.17 (m, 2H), 4.94- 4.88 (m, 1H), 4.55- 4.48 (m, 1H), 4.14- 3.45 (m, 11H), 2.48- 2.44 (m, 3H), 2.37- 1.79 (m, 6H) |
| 370 | (fluoro(7-(((5S,8S, 10aR)-3-(5- methylbenzo[d] isoxazole-3- carbonyl)-6-oxo-((S)- 3-phenylmorpholine- 4-carbonyl)decahydro- pyrrolo[1,2-a] [1,5]diazocin-5- yl)carbamoyl) naphthalen-2-yl) methyl)phosphonic acid | 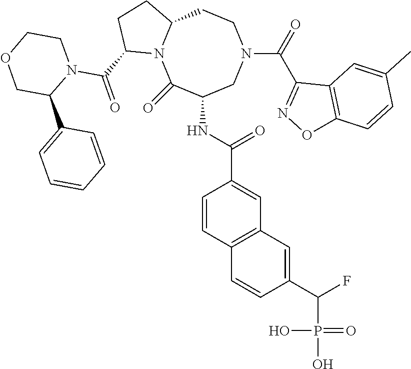 [SMILES: Cc1ccc2onc(C(=O)N3CC[C@H]4CC[C@@H](C(=O)N5CCOC[C@@H]5c5ccccc5)N4C(=O)[C@@H](NC(=O)c4ccc5ccc(C(F)P(=O)(O)O)cc5c4)C3)c2c1] | 798.3 [M + H]⁺ | ¹H NMR (400 MHz, DMSO-d₆) δ 9.15- 8.83 (m, 1H), 8.68- 8.47 (m, 1H), 8.32- 7.80 (m, 5H), 7.77- 7.08 (m, 8H), 6.03- 5.84 (m, 1H), 5.43- 5.33 (m, 1H), 4.85- 4.76 (m, 1H), 4.60- 4.46 (m, 2H), 4.28- 3.71 (m, 7H), (TFA salt) |
| 371 | (fluoro(7-(((5S,8S,10aR)- 3-(5-methylbenzo[d] isoxazole-3-carbonyl)- 6-oxo-8-((R)-3- phenylmorpholine-4- carbonyl)decahydro- pyrrolo[1,2-a][1,5] diazocin-5-yl) carbamoyl)naphthalen- 2-yl)methyl)phosphonic acid | 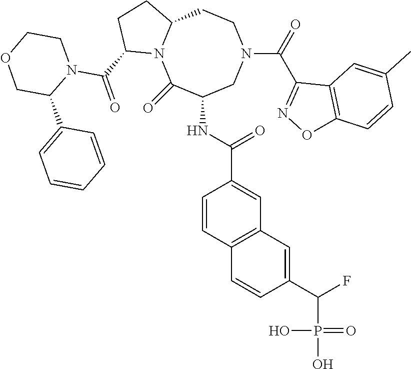 [SMILES: Cc1ccc2onc(C(=O)N3CC[C@H]4CC[C@@H](C(=O)N5CCOC[C@H]5c5ccccc5)N4C(=O)[C@@H](NC(=O)c4ccc5ccc(C(F)P(=O)(O)O)cc5c4)C3)c2c1] | 797.8 [M + H]⁺ | ¹H NMR (400 MHz, DMSO-d₆) δ 9.07- 8.80 (m, 1H), 8.66- 8.45 (m, 1H), 8.12- 7.86 (m, 5H), 7.79- 7.05 (m, 8H), 6.03- 5.83 (m, 1H), 5.40- 5.12 (m, 2H), 5.00- 4.78 (m, 1H), 4.62- 4.48 (m, 2H), 4.44- 4.34 (m, 1H), 4.15- 4.09 (m, 1H), 3.94- 3.64 (m, 4H), 3.62- 2.99 (m, 3H), 2.46- 2.30 (m, 3H), 2.29- 2.03 (m, 3H), 1.98- 1.55 (m, 3H) (TFA salt) |
| 372 | ((7-(((5S,8S,10aR)-8- ((1S,4S)-2-oxa-5- azabicyclo[2.2.1]heptane- 5-carbonyl)-3-(5- methylbenzo[d] isoxazole-3-carbonyl)-6- oxodecahydropyrrolo [1,2-a][1,5]diazocin-5- yl)carbamoyl) naphthalen-2-yl) fluoromethyl)phosphonic acid | 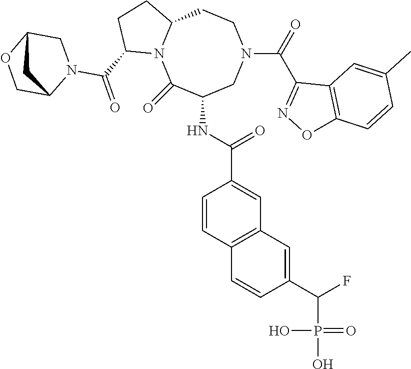 [SMILES: Cc1ccc2onc(C(=O)N3CC[C@H]4CC[C@@H](C(=O)N5C[C@@H]6C[C@H]5CO6)N4C(=O)[C@@H](NC(=O)c4ccc5ccc(C(F)P(=O)(O)O)cc5c4)C3)c2c1] | 734.4 [M + H]⁺ | ¹H NMR (400 MHz, DMSO-d₆) δ 9.04- 8.57 (m, 1H), 8.53- 8.41 (m, 1H), 8.10- 7.82 (m, 5H), 7.77- 7.62 (m, 2H), 7.59- 7.47 (m, 1H), 6.02- 5.76 (m, 1H), 5.35- 5.16 (m, 1H), 4.81- 4.71 (m, 1H), 4.66- 4.60 (m, 1H), 4.55- 4.44 (m, 2H), 4.38- 4.33 (m, 1H), 4.27- 4.21 (m, 1H), 4.01- 3.96 (m, 2H), 3.69- 3.51 (m, 2H), 3.38- 3.02 (m, 2H), 2.47- 2.40 (m, 3H), 2.39- 2.19 (m, 2H), 2.19- 1.50 (m, 6H) |
| 373 | ((7-(((5S,8S,10aR)-8- (2-benzylmorpholine-4- carbonyl)-3-(5- methylbenzo[d] isoxazole-3-carbonyl)- 6-oxodecahydropyrrolo [1,2-a][1,5]diazocin-5- yl)carbamoyl)naphthalen- 2-yl)fluoromethyl) phosphonic acid | 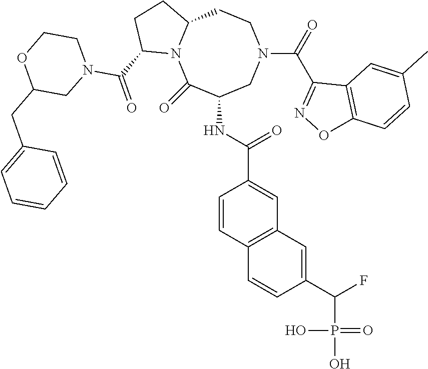 [SMILES: Cc1ccc2onc(C(=O)N3CC[C@H]4CC[C@@H](C(=O)N5CCOC(Cc6ccccc6)C5)N4C(=O)[C@@H](NC(=O)c4ccc5ccc(C(F)P(=O)(O)O)cc5c4)C3)c2c1] | 812.3 [M + H]⁺ | ¹H NMR (400 MHz, DMSO-d₆) δ 9.04- 8.69 (m, 1H), 8.64- 8.43 (m, 1H), 8.14- 7.84 (m, 5H), 7.76- 7.62 (m, 2H), 7.59- 7.47 (m, 1H), 7.32- 7.08 (m, 5H), 6.01- 5.82 (m, 1H), 5.30- 5.14 (m, 1H), 4.89- 4.77 (m, 1H), 4.71- 4.63 (m, 1H), 4.53- 4.46 (m, 1H), 4.34- 4.18 (m, 1H), 4.08- 4.00 (m, 2H), 3.92- 3.81 (m, 2H), 3.69- 2.86 (m, 4H), 2.82- 2.55 (m, 3H), 2.47- 2.40 (m, 3H), 2.39- 2.02 (m, 3H), 1.99- 1.78 (m, 2H), 1.72- 1.48 (m, 1H) |
| 374 | ((7-(((5S,8S,10aR)-3-(5- ethylbenzo[d]isoxazole- 3-carbonyl)-6-oxo-8-(7- oxa-4-azaspiro[2.5] octane-4-carbonyl) decahydropyrrolo[1,2- a][1,5]diazocin-5-yl) carbamoyl)naphthalen- 2-yl)fluoromethyl) phosphonic acid | 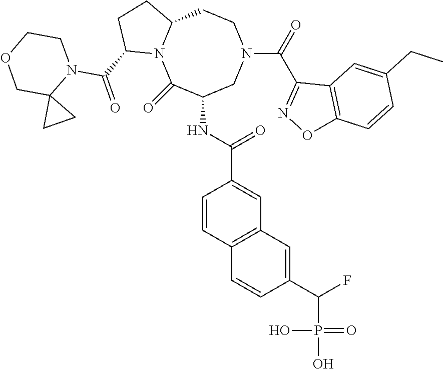 [SMILES: CCc1ccc2onc(C(=O)N3CC[C@H]4CC[C@@H](C(=O)N5CCOCC56CC6)N4C(=O)[C@@H](NC(=O)c4ccc5ccc(C(F)P(=O)(O)O)cc5c4)C3)c2c1] | 762.2 [M + H]⁺ | ¹H NMR (400 MHz, DMSO-d₆) δ 9.11- 8.46 (m, 2H), 8.12- 7.82 (m, 5H), 7.80- 7.64 (m, 2H), 7.62- 7.51 (m, 1H), 6.05- 5.83 (m, 1H), 5.38- 5.25 (m, 1H), 4.99- 4.90 (m, 1H), 4.59- 3.55 (m, 9H), 3.41- 3.09 (m, 2H), 2.80- 2.69 (m, 2H), 2.36- 1.50 (m, 6H), 1.31- 0.47 (m, 7H) |
| 375 | ((7- (((5S,8S,10aR)- 8- ((3S,4R)- 3- cyano-4- phenylpyrrolidine- 1- carbonyl)-3- (5-methyl- benzo[d] isoxazole- 3- carbonyl)- 6- oxodecahydropyrrolo [1,2-a] [1,5] diazocin- 5-yl) carbamoyl) naphthalen- 2-yl) difluoromethyl) phosphonic acid or | 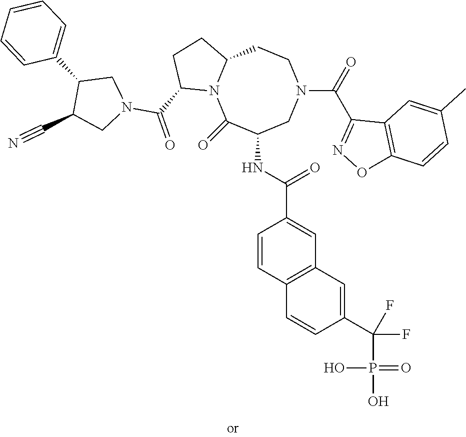 [SMILES: Cc1ccc2onc(C(=O)N3CC[C@H]4CC[C@@H](C(=O)N5C[C@@H](C#N)[C@H](c6ccccc6)C5)N4C(=O)[C@@H](NC(=O)c4ccc5ccc(C(F)(F)P(=O)(O)O)cc5c4)C3)c2c1] | 825.3 [M + H]⁺ | ¹H NMR (400 MHz, DMSO-d₆) δ 9.14- 8.70 (m, 1H), 8.62- 8.42 (m, 1H), 8.30- 8.11 (m, 2H), 8.10- 8.03 (m, 2H), 8.01- 7.92 (m, 1H), 7.79- 7.60 (m, 2H), 7.58- 7.43 (m, 1H), 7.43- 7.35 (m, 3H), 7.35- 6.97 (m, 2H), 5.38- 5.17 (m, 1H), 4.90- 4.40 (m, 3H), 4.19- 4.14 (m, 2H), 3.65- 3.47 (m, 5H), 3.46- 3.36 (m, 1H), 3.35- 3.17 (m, 1H), 2.48- 2.29 (m, 4H), 2.21- 1.98 (m, 2H), 1.96- 1.53 (m, 3H) |
| | ((7-(((5S,8S,10aR)-8- ((3R,4S)-3-cyano-4- phenylpyrrolidine- 1-carbonyl)-3-(5- methylbenzo[d] isoxazole-3- carbonyl)-6- oxodecahydropyrrolo [1,2-a][1,5] diazocin-5- yl)carbamoyl) naphthalen-2- yl) difluoromethyl) phosphonic acid | 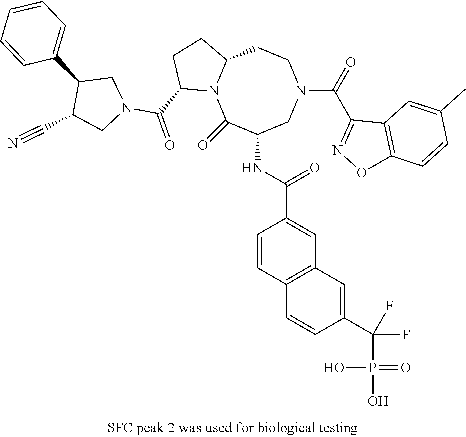 [SMILES: Cc1ccc2onc(C(=O)N3CC[C@H]4CC[C@@H](C(=O)N5C[C@H](c6ccccc6)[C@@H](C#N)C5)N4C(=O)[C@@H](NC(=O)c4ccc5ccc(C(F)(F)P(=O)(O)O)cc5c4)C3)c2c1] | | |
| 376 | (fluoro(6-(((5S,8S, 10aR)-3-(5- methylbenzo[d] isoxazole-3-carbonyl)- 8-(morpholine-4- carbonyl)-6- oxodecahydropyrrolo [1,2-a][1,5]diazocin-5- yl)carbamoyl)quinolin- 3-yl)methyl)phosphonic acid | 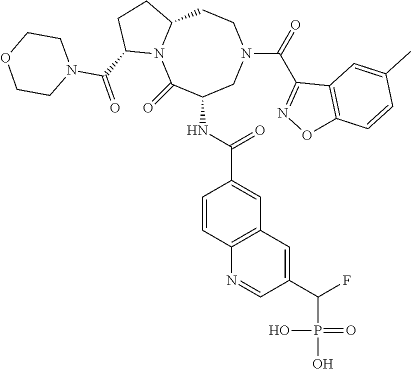 [SMILES: Cc1ccc2onc(C(=O)N3CC[C@H]4CC[C@@H](C(=O)N5CCOCC5)N4C(=O)[C@@H](NC(=O)c4ccc5ncc(C(F)P(=O)(O)O)cc5c4)C3)c2c1] | 723.5 [M + H]⁺ | ¹H NMR (400 MHz, DMSO-d₆) δ 9.06- 8.81 (m, 2H), 8.70- 8.53 (m, 1H), 8.53- 8.42 (m, 1H), 8.30- 8.07 (m, 2H), 7.92 (s, 1H), 7.77-7.67 (m, 1H), 7.58-7.49 (m, 1H), 6.19-5.96 (m, 1H), 5.32-5.16 (m, 1H), 4.87-4.76 (m, 1H), 4.51-4.43 (m, 1H), 4.26-4.21 (m, 1H), 4.04-3.99 (m, 2H), 3.89-3.83 (m, 2H), 3.58-3.44 (m, 6H), 3.32-3.21 (m, 1H), 2.46-2.41 (m, 3H) 2.31-1.97 (m, 3H), 1.95-1.59 (m, 3H) |
| 377 | ((1R)-(7-(((5S,8S,10aR)- 8-(8-oxa-3- azabicyclo[3.2.1]octane- 3-carbonyl)-3-(5- methylbenzo[d] isoxazole-3-carbonyl)-6- oxodecahydropyrrolo [1,2-a][1,5]diazocin-5- yl)carbamoyl) naphthalen-2-yl) fluoromethyl)phosphonic acid | 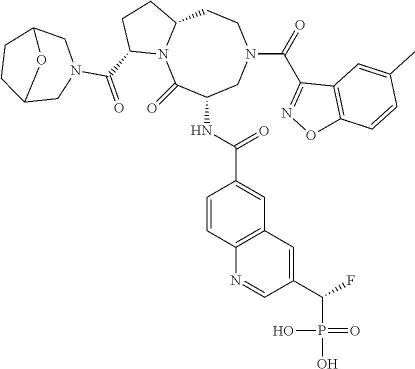 [SMILES: Cc1ccc2onc(C(=O)N3CC[C@H]4CC[C@@H](C(=O)N5CC6CCC(C5)O6)N4C(=O)[C@@H](NC(=O)c4ccc5ncc([C@H](F)P(=O)(O)O)cc5c4)C3)c2c1] | 748.3 [M + H]⁺ | ¹H NMR (400 MHz, DMSO-d₆) δ 9.09- 8.68 (m, 1H), 8.65- 8.45 (m, 1H), 8.11- 7.88 (m, 5H), 7.76- 7.63 (m, 2H), 7.58- 7.48 (m, 1H), 6.02- 5.79 (m, 1H), 5.38- 5.18 (m, 1H), 4.83- 4.63 (m, 1H), 4.51- 4.44 (m, 1H), 4.32- 4.27 (m, 1H), 4.23- 4.15 (m, 2H), 4.01- 3.93 (m, 2H), 3.89- 3.78 (m, 2H), 3.71- 3.57 (m, 1H), 3.26- 3.15 (m, 1H), 2.87- 2.64 (m, 1H), 2.47- 2.42 (m, 3H), 2.37- 2.05 (m, 3H), 2.00- 1.79 (m, 3H), 1.78- 1.46 (m, 4H) (TFA Salt) |
| 378 | ((5-cyano-7-(((5S,8S, 10aR)-3-(5- methylbenzo[d] isoxazole-3- carbonyl)-8-(morpholine- 4-carbonyl)-6- oxodecahydropyrrolo [1,2-a][1,5]diazocin-5- yl)carbamoyl) naphthalen-2-yl)methyl) phosphonic acid | 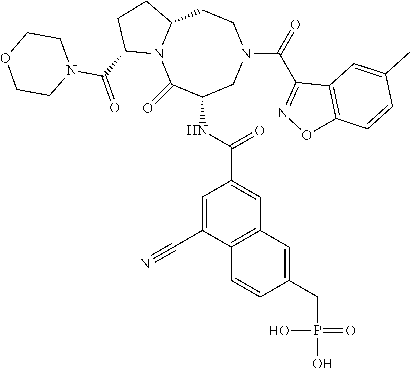 [SMILES: Cc1ccc2onc(C(=O)N3CC[C@H]4CC[C@@H](C(=O)N5CCOCC5)N4C(=O)[C@@H](NC(=O)c4cc(C#N)c5ccc(CP(=O)(O)O)cc5c4)C3)c2c1] | 729.3 [M + H]⁺ | ¹H NMR (400 MHz, DMSO-d₆) δ 9.21- 8.90 (m, 1H), 8.89- 8.68 (m, 1H), 8.59- 8.40 (m, 1H), 8.15- 8.00 (m, 2H), 7.93 (s, 1H), 7.86-7.76 (m, 1H), 7.76-7.64 (m, 1H), 7.58-7.46 (m, 1H), 5.31-5.18 (m, 1H), 4.84-4.77 (m, 1H), 4.53-4.46 (m, 1H), 4.21-3.85 (m, 4H), 3.62-3.27 (m, 8H), 3.27-3.16 (m, 2H), 2.47-2.39 (m, 3H), 2.35-1.84 (m, 4H), 1.83-1.54 (m, 2H) |
| 379 | ((4-fluoro-7-(((5S,8S, 10aR)-3-(5- methylbenzo[d] isoxazole-3-carbonyl)- 8-(morpholine-4- carbonyl)-6- oxodecahydropyrrolo [1,2-a][1,5]diazocin-5- yl)carbamoyl) naphthalen-2-yl) methyl)phosphonic acid | 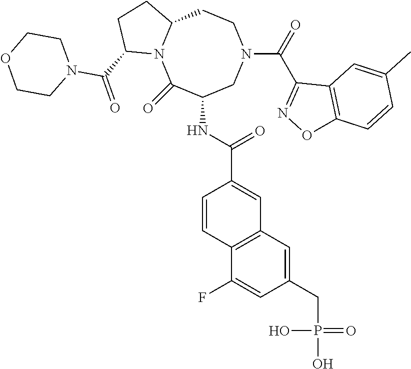 [SMILES: Cc1ccc2onc(C(=O)N3CC[C@H]4CC[C@@H](C(=O)N5CCOCC5)N4C(=O)[C@@H](NC(=O)c4ccc5c(F)cc(CP(=O)(O)O)cc5c4)C3)c2c1] | 722.3 [M + H]⁺ | ¹H NMR (400 MHz, DMSO-d₆) δ 9.05- 8.72 (m, 1H), 8.51 (d, J = 51.3 Hz, 1H), 8.14- 8.02 (m, 1H), 7.94 (d, J = 10.2 Hz, 2H), 7.78-7.62 (m, 2H), 7.59-7.45 (m, 1H), 7.41-7.27 (m, 1H), 5.22 (d, J = 31.2 Hz, 1H), 4.86-4.77 (m, 1H), 4.51-4.44 (m, 1H), 4.36-3.77 (m, 7H), 3.56-3.47 (m, 4H), 3.35-3.08 (m, 3H), 2.44 (d, J = 11.6 Hz, 3H), 2.35-1.99 (m, 3H), 1.96-1.55 (m, 3H) |
| 380 | ((S)-fluoro(7-(((5S,8S, 10aR)-8-((R)-6-(1- methyl-2-oxo-1,2- dihydropyridin-4-yl)- 4-azaspiro[2.4]heptane- 4-carbonyl)-3-(5- methylbenzo[d] isoxazole-3-carbonyl)- 6-oxodecahydropyrrolo [1,2-a][1,5] diazocin-5-yl) carbamoyl)naphthalen- 2-yl)methyl)phosphonic acid | 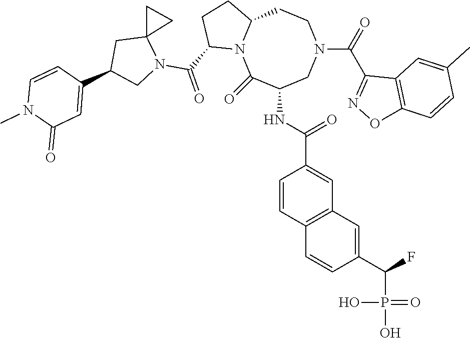 | 839.4 [M + H]⁺ | ¹H NMR (400 MHz, DMSO-d₆) δ 9.08- 8.75 (m, 1H), 8.68- 8.44 (m, 1H), 8.11- 7.45 (m, 9H), 6.34- 6.15 (m, 2H), 6.01- 5.83 (m, 1H), 5.33- 5.20 (m, 1H), 4.57- 4.40 (m, 2H), 4.31- 4.17 (m, 2H), 4.07- 3.86 (m, 3H), 3.57- 3.50 (m, 1H), 3.39- 3.32 (m, 3H), 3.28- 3.17 (m, 1H), 2.49- 2.44 (m, 3H), 2.35- 2.01 (m, 4H), 2.00- 1.52 (m, 6H), 0.52- 0.12 (m, 2H) (TFA salt) |
| 381 | (difluoro(7-(((5S,8S, 10aR)-3-(5- methylbenzo[d] isoxazole-3-carbonyl)- 6-oxo-8-((S)-6-(2-oxo- 1,2-dihydropyridin-4- yl)-4-azaspiro[2.4] heptane-4-carbonyl) decahydropyrrolo [1,2-a][1,5]diazocin-5- yl)carbamoyl) naphthalen-2-yl) methyl)phosphonic acid | 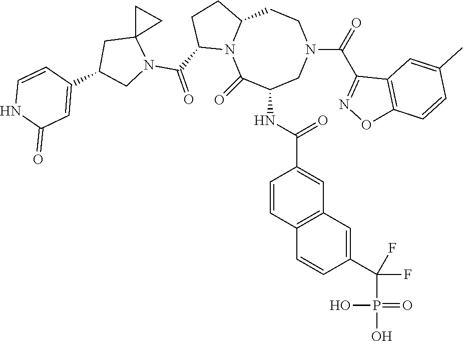 [SMILES: Cc1ccc2onc(C(=O)N3CC[C@H]4CC[C@@H](C(=O)N5C[C@H](c6cc[nH]c(=O)c6)CC56CC6)N4C(=O)[C@@H](NC(=O)c4ccc5ccc(C(F)(F)P(=O)(O)O)cc5c4)C3)c2c1] | 843.3 [M + H]⁺ | ¹H NMR (400 MHz, DMSO-d₆) δ 9.05- 8.79 (m, 1H), 8.75- 8.58 (m, 1H), 8.26- 8.17 (m, 1H), 8.17- 7.88 (m, 4H), 7.82- 7.67 (m, 3H), 7.57- 7.49 (m, 1H), 7.34- 7.27 (m, 1H), 6.27- 6.22 (m, 1H), 6.21- 6.15 (m, 1H), 5.31- 5.23 (m, 1H), 4.57- 4.52 (m, 1H), 4.45- 4.39 (m, 1H), 4.32- 4.24 (m, 1H), 4.22- 4.17 (m, 1H), 4.05- 3.84 (m, 3H), 3.59- 3.48 (m, 1H), 3.33- 3.18 (m, 1H), 2.47 (s, 3H), 2.36-2.06 (m, 4H), 1.97-1.79 (m, |
| | | | | 4H), 1.69-1.53 (m, |
| | | | | 1H), 1.20-1.11 (m, |
| | | | | 1H), 0.49-0.13 (m, |
| | | | | 2H) |
| 382 | (difluoro(7-(((5S,8S, 10aR)-8-((S)-6-(1- methyl-2-oxo-1,2- dihydropyridin-4-yl)- 4-azaspiro[2.4]heptane- 4-carbonyl)-3-(5- methylbenzo[d] isoxazole-3-carbonyl)-6- oxodecahydropyrrolo [1,2-a][1,5]diazocin-5- yl)carbamoyl) naphthalen-2-yl) methyl)phosphonic acid | 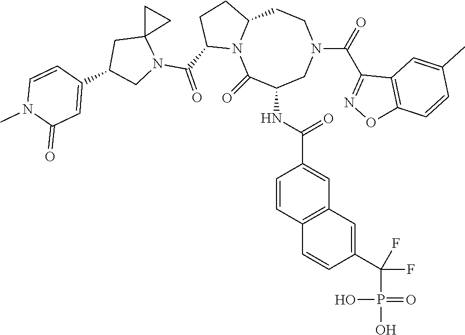 | 857.2 [M + H]⁺ | ¹H NMR (400 MHz, DMSO-d₆) δ 9.05- 8.78 (m, 1H), 8.76- 8.58 (m, 1H), 8.30- 8.16 (m, 1H), 8.14- 8.08 (m, 1H), 8.08- 7.95 (m, 2H), 7.82- 7.75 (m, 1H), 7.74- 7.65 (m, 2H), 7.61- 7.48 (m, 2H), 6.35- 6.27 (m, 1H), 6.23- 6.14 (m, 1H), 5.31- 5.23 (m, 1H), 4.53- 4.49 (m, 1H), 4.44- 4.39 (m, 1H), 4.18- 4.08 (m, 1H), 4.04- 3.97 (m, 2H), 3.96- 3.79 (m, 2H), 3.71- 3.57 (m, 1H), 3.39- 3.26 (m, 4H), 2.47 (s, 3H), 2.39-2.05 (m, |
| | | | | 3H), 2.05-1.89 (m, |
| | | | | 3H), 1.87-1.67 (m, |
| | | | | 2H), 1.66-1.49 (m, |
| | | | | 2H), 0.51-0.11 (m, |
| | | | | 2H) |
| 383 | (difluoro(7-(((5S,8S, 10aR)-8-((R)-6-(1- methyl-2-oxo-1,2- dihydropyridin-4-yl)-4- azaspiro[2.4]heptane-4- carbonyl)-3-(5- methylbenzo[d] isoxazole-3-carbonyl)-6- oxodecahydropyrrolo [1,2-a][1,5]diazocin-5- yl)carbamoyl) naphthalen-2-yl) methyl)phosphonic acid | 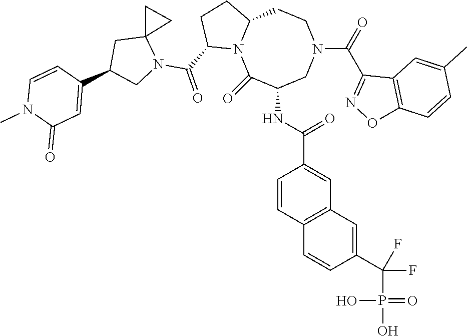 | 857.3 [M + H]⁺ | ¹H NMR (400 MHz, DMSO-d₆) δ 9.05- 8.78 (m, 1H), 8.74- 8.59 (m, 1H), 8.25- 8.16 (m, 1H), 8.12- 7.96 (m, 3H), 7.81- 7.49 (m, 5H), 6.32- 6.26 (m, 1H), 6.23- 6.18 (m, 1H), 5.30- 5.22 (m, 1H), 4.55- 4.40 (m, 2H), 4.28- 4.17 (m, 2H), 4.04- 3.88 (m, 3H), 3.56- 3.51 (m, 1H), 3.38- 3.33 (m, 3H), 3.26- 3.18 (m, 1H), 2.49- 2.45 (m, 3H), 2.33- 2.04 (m, 4H), 1.98- 1.57 (m, 6H), 0.51- 0.15 (m, 2H) |
| 384 | ((4-cyano-7-(((5S,8S, 10aR)-3-(5- methylbenzo[d] isoxazole-3-carbonyl)- 8-(morpholine-4- carbonyl)-6- oxodecahydropyrrolo [1,2-a][1,5]diazocin-5- yl)carbamoyl) naphthalen-2-yl)methyl) phosphonic acid | 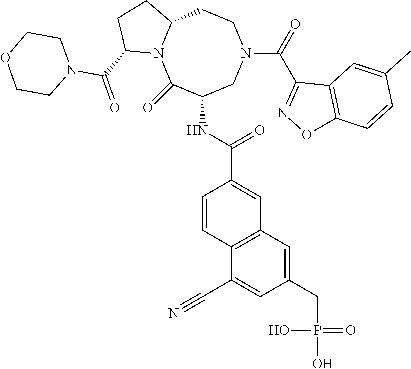 [SMILES: Cc1ccc2onc(C(=O)N3CC[C@H]4CC[C@@H](C(=O)N5CCOCC5)N4C(=O)[C@@H](NC(=O)c4ccc5c(C#N)cc(CP(=O)(O)O)cc5c4)C3)c2c1] | 729.1 [M + H]⁺ | ¹H NMR (400 MHz, DMSO-d₆) δ 9.13- 8.84 (m, 1H), 8.71- 8.49 (m, 1H), 8.30- 8.01 (m, 4H), 7.92 (s, 1H), 7.78-7.65 (m, 1H), 7.59-7.45 (m, 1H), 5.36-5.15 (m, 1H), 4.89-4.76 (m, 1H), 4.54-4.42 (m, 1H), 4.29-3.84 (m, 4H), 3.59-3.18 (m, 10H) 2.47-2.39 (m, 3H), 2.34-1.86 (m, 4H), 1.83-1.55 (m, 2H) |
| 385 | (difluoro(7-(((5S,8S, 10aR)-3-(5-methyl- benzo[d]isoxazole-3- carbonyl)-6-oxo-8-((R)- 6-(2-oxo-1,2-dihydro- pyridin-4-yl)-4-azaspiro [2.4]heptane-4- carbonyl)decahydro- pyrrolo[1,2-a][1,5] diazocin-5-yl) carbamoyl)naphthalen- 2-yl)methyl) phosphonic acid | 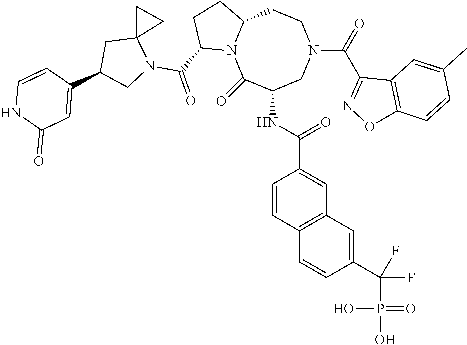 [SMILES: Cc1ccc2onc(C(=O)N3CC[C@H]4CC[C@@H](C(=O)N5C[C@@H](c6cc[nH]c(=O)c6)CC56CC6)N4C(=O)[C@@H](NC(=O)c4ccc5ccc(C(F)(F)P(=O)(O)O)cc5c4)C3)c2c1] | 841.3 [M − H]⁻ | ¹H NMR (400 MHz, DMSO-d₆) δ 9.04- 8.80 (m, 1H), 8.75- 8.60 (m, 1H), 8.26- 8.15 (m, 1H), 8.13- 7.95 (m, 3H), 7.84- 7.66 (m, 3H), 7.58- 7.49 (m, 1H), 7.30- 7.18 (m, 1H), 6.28- 6.21 (m, 1H), 6.21- 6.12 (m, 1H), 5.34- 5.21 (m, 1H), 4.52- 4.42 (m, 2H), 4.19- 4.10 (m, 2H), 4.04- 3.99 (m, 2H), 3.93- 3.87 (m, 1H), 3.66- 3.58 (m, 1H), 3.36- 3.27 (m, 1H), 2.47 (s, 3H), 2.37-1.91 (m, 6H), 1.84-1.43 (m, 4H), 0.50-0.12 (m, 2H) (TFA salt) |
| 386 | ((R)-(7-(((5S,8S, 10aR)-8-(5,7-dioxo-6- (pyridin-3-yl)-4,6- diazaspiro[2.4]heptane- 4-carbonyl)-3-(5- methylbenzo[d] isoxazole-3-carbonyl)- 6-oxodecahydropyrrolo [1,2-a][1,5]diazocin-5- yl)carbamoyl) naphthalen-2-yl) fluoromethyl)phosphonic acid | 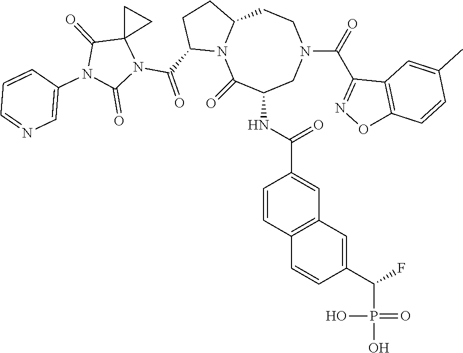 [SMILES: Cc1ccc2onc(C(=O)N3CC[C@H]4CC[C@@H](C(=O)N5C(=O)N(c6cccnc6)C(=O)C56CC6)N4C(=O)[C@@H](NC(=O)c4ccc5ccc([C@H](F)P(=O)(O)O)cc5c4)C3)c2c1] | 838.3 [M + H]⁺ | ¹H NMR (400 MHz, DMSO-d₆) δ 9.12- 8.88 (m, 1H), 8.71- 8.42 (m, 3H), 8.09- 7.50 (m, 10H), 6.00- 5.74 (m, 1H), 5.42- 5.27 (m, 2H), 4.70- 4.44 (m, 2H), 4.10- 4.01 (m, 1H), 3.92- 3.86 (m, 1H), 3.81- 3.73 (m, 1H), 2.43- 0.80 (m, 13H) |
| 387 | ((R)-fluoro(7-(((5S, 8S,10aR)-8-((R)-6- (1-methyl-2-oxo-1,2- dihydropyridin-4-yl)-4- azaspiro[2.4]heptane-4- carbonyl)-3-(5- methylbenzo[d] isoxazole-3-carbonyl)- 6-oxodecahydro- pyrrolo[1,2-a][1,5] diazocin-5-yl) carbamoyl)naphthalen- 2-yl)methyl) phosphonic acid | 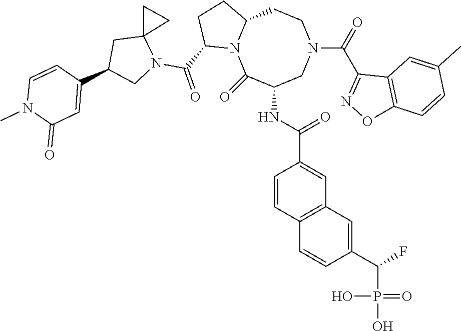 | 839.3 [M + H]⁺ | ¹H NMR (400 MHz, DMSO-d₆) δ 9.07- 8.78 (m, 1H), 8.66- 8.47 (m, 1H), 8.12- 7.48 (m, 9H), 6.32- 6.27 (m, 1H), 6.24- 6.18 (m, 1H), 5.98- 5.86 (m, 1H), 5.29- 5.24 (m, 1H), 4.59- 4.52 (m, 1H), 4.47- 4.39 (m, 1H), 4.31- 3.85 (m, 5H), 3.59- 3.48 (m, 1H), 3.40- 3.33 (m, 3H), 3.32- 3.18 (m, 1H), 2.50- 2.43 (m, 3H), 2.40- 1.43 (m, 10H), 0.52- 0.14 (m, 2H). |
| 388 | ((5-methoxy-7-(((5S, 8S,10aR)-3-(5- methylbenzo[d] isoxazole-3-carbonyl)- 8-(morpholine-4- carbonyl)-6- oxodecahydropyrrolo [1,2-a][1,5]diazocin-5- yl)carbamoyl) naphthalen-2-yl)methyl) phosphonic acid | 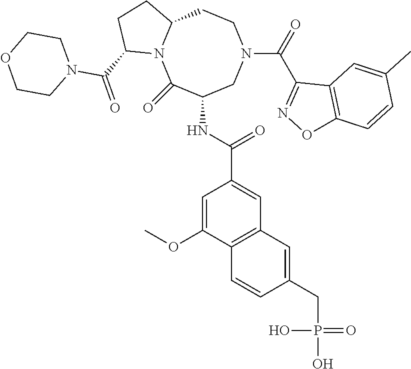 [SMILES: COc1cc(C(=O)N[C@H]2CN(C(=O)c3noc4ccc(C)cc34)CC[C@H]3CC[C@@H](C(=O)N4CCOCC4)N3C2=O)cc2cc(CP(=O)(O)O)ccc12] | 734.3 [M + H]⁺ | ¹H NMR (400 MHz, DMSO-d₆) δ 8.97- 8.64 (m, 1H), 8.14- 7.87 (m, 3H), 7.86- 7.75 (m, 1H), 7.75- 7.66 (m, 1H), 7.60- 7.42 (m, 2H), 7.39- 7.23 (m, 1H), 5.33- 5.17 (m, 1H), 4.85- 4.77 (m, 1H), 4.52- 4.45 (m, 1H), 4.25- 4.12 (m, 2H), 3.98 (s, 3H), 3.92-3.82 (m, 2H), 3.63-3.39 (m, 8H), 3.19-3.08 (m, 2H), 2.48-2.33 (m, 3H), 2.33-1.85 (m, 4H), 1.84-1.57 (m, 2H) |
| 389 | ((7-(((5S,8S,10aR)-2- (3-chloro-5-methyl- benzoyl)-8-(morpholine- 4-carbonyl)-6- oxodecahydropyrrolo [1,2-a][1,4]diazocin-5- yl)carbamoyl)naphthalen- 2-yl)fluoromethyl) phosphonic acid | 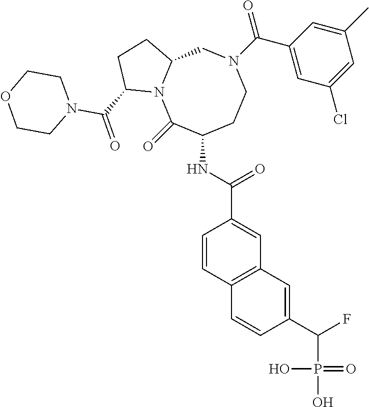 [SMILES: Cc1cc(Cl)cc(C(=O)N2CC[C@H](NC(=O)c3ccc4ccc(C(F)P(=O)(O)O)cc4c3)C(=O)N3[C@H](CC[C@H]3C(=O)N3CCOCC3)C2)c1] | 715.3 [M + H]⁺ | ¹H NMR (400 MHz, DMSO-d₆) δ 8.88- 8.62 (m, 1H), 8.58- 8.47 (m, 1H), 8.08- 7.93 (m, 4H), 7.71- 7.64 (m, 1H), 7.42- 7.21 (m, 3H), 6.00- 5.85 (m, 1H), 5.08- 4.91 (m, 3H), 4.52- 4.48 (m, 1H), 4.41- 4.37 (m, 1H), 4.06- 3.98 (m, 1H), 3.62- 3.43 (m, 8H), 3.40- 3.20 (m, 1H), 2.42- 1.41 (m, 9H) |
| 390 | ((R)-fluoro(7-(((5S, 8S,10aR)-8-((S)-6- (1-methyl-2-oxo-1,2- dihydropyridin-4-yl)-4- azaspiro[2.4]heptane-4- carbonyl)-3-(5- methylbenzo[d] isoxazole-3-carbonyl)-6- oxodecahydropyrrolo [1,2-a][1,5]diazocin-5- yl)carbamoyl) naphthalen-2-yl) methyl)phosphonic acid | 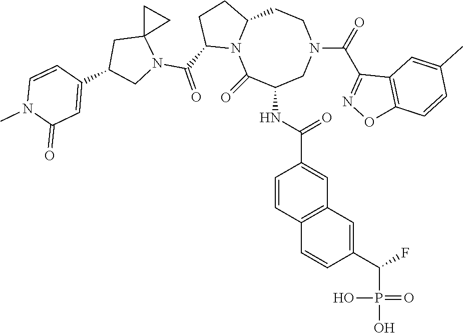 | 839.2 [M + H]⁺ | ¹H NMR (400 MHz, DMSO-d₆) δ 9.05- 8.78 (m, 1H), 8.66- 8.48 (m, 1H), 8.09- 7.93 (m, 4H), 7.82- 7.62 (m, 3H), 7.61- 7.49 (m, 2H), 6.38- 6.26 (m, 1H), 6.26- 6.13 (m, 1H), 6.00- 5.82 (m, 1H), 5.31- 5.18 (m, 1H), 4.54- 4.48 (m, 1H), 4.45- 4.38 (m, 1H), 4.21- 4.09 (m, 2H), 4.01- 3.99 (m, 1H), 3.89- 3.86 (m, 1H), 3.61- 3.60 (m, 1H), 3.34 (s, 1H), 3.32 (s, 3H), 2.46 (s, 3H), 2.41-2.27 (m, 1H), 2.23-2.06 (m, 2H), 2.06-1.92 (m, |
| | | | | 3H), 1.85-1.71 (m, |
| | | | | 2H), 1.65-1.50 (m, |
| | | | | 2H), 1.45-1.23 (m, |
| | | | | 1H), 0.53-0.14 (m, |
| | | | | 2H) |
| 391 | ((S)-fluoro(7-(((5S, 8S,10aR)-8-((S)-6- (1-methyl-2-oxo-1,2- dihydropyridin-4-yl)-4- azaspiro[2.4]heptane-4- carbonyl)-3-(5- methylbenzo[d] isoxazole-3-carbonyl)-6- oxodecahydropyrrolo [1,2-a][1,5]diazocin-5- yl)carbamoyl) naphthalen-2-yl)methyl) phosphonic acid | 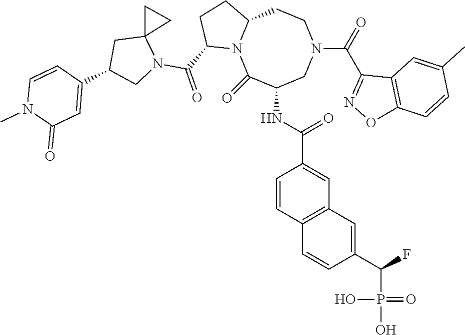 | 839.2 [M + H]⁺ | ¹H NMR (400 MHz, DMSO-d₆) δ 9.06- 8.76 (m, 1H), 8.65- 8.48 (m, 1H), 8.09- 7.92 (m, 4H), 7.82- 7.63 (m, 3H), 7.60- 7.50 (m, 2H), 6.37- 6.27 (m, 1H), 6.24- 6.15 (m, 1H), 6.01- 5.83 (m, 1H), 5.32- 5.18 (m, 1H), 4.55- 4.48 (m, 1H), 4.45- 4.38 (m, 1H), 4.19- 4.10 (m, 2H), 4.02- 4.00 (m, 1H), 3.62- 3.60 (m, 1H), 3.41- 3.36 (m, 1H), 3.34 (s, 1H), 3.32 (s, 3H), 2.46 (s, 3H), 2.39-2.25 (m, 1H), 2.23-2.08 (m, 2H), 2.04-1.90 (m, |
| | | | | 3H), 1.86-1.71 (m, |
| | | | | 2H), 1.69-1.38 (m, |
| | | | | 3H), 0.49-0.12 (m, |
| | | | | 2H) |
| 392 | (fluoro(7-(((5S,8S, 10aR)-3-(isoquinolin- 3-yl)-8-(morpholine- 4-carbonyl)-6- oxodecahydropyrrolo [1,2-a][1,5]diazocin-5- yl)carbamoyl)naphthalen- 2-yl)methyl)phosphonic acid | 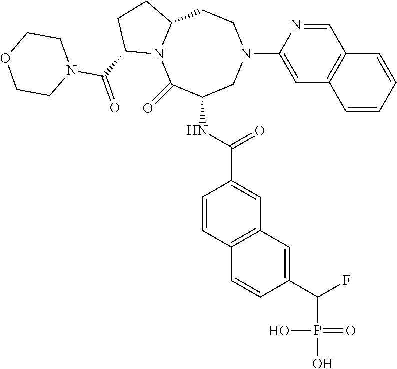 [SMILES: O=C(N[C@H]1CN(c2cc3ccccc3cn2)CC[C@H]2CC[C@@H](C(=O)N3CCOCC3)N2C1=O)c1ccc2ccc(C(F)P(=O)(O)O)cc2c1] | 690.3 [M + H]⁺ | ¹H NMR (400 MHz, DMSO-d₆) δ 8.99 (s, 1H), 8.72-8.56 (m, 2H), 8.19-7.98 (m, 4H), 7.92-7.82 (m, 1H), 7.78-7.62 (m, 2H), 7.59-7.50 (m, 1H), 7.42-7.33 (m, 1H), 7.28-7.19 (m, 1H), 6.03-5.79 (m, 1H), 5.10-5.00 (m, 1H), 4.93-4.77 (m, 1H), 4.47-4.21 (m, 2H), 4.07-3.96 (m, 1H), 3.82-3.69 (m, 1H), 3.68-3.34 (m, 9H), 2.39-2.21 (m, 2H), 2.01-1.59 (m, 4H) |
| 393 | (fluoro(7-(((4S,7S,9aR)- 7-(morpholine-4- carbonyl)-5-oxo-2- (pyridin-2-yl)octahydro- 1H-pyrrolo[1,2-a][1,4] diazepin-4- yl)carbamoyl) naphthalen-2-yl)methyl) phosphonic acid | 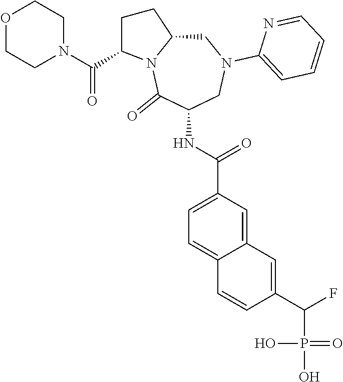 [SMILES: O=C(N[C@H]1CN(c2ccccn2)C[C@H]2CC[C@@H](C(=O)N3CCOCC3)N2C1=O)c1ccc2ccc(C(F)P(=O)(O)O)cc2c1] | 626.3 [M + H]⁺ | ¹H NMR (400 MHz, DMSO-d₆) δ 9.15- 8.97 (m, 1H), 8.13- 8.08 (m, 1H), 8.07- 7.97 (m, 2H), 7.97- 7.92 (m, 1H), 7.90- 7.72 (m, 1H), 7.71- 7.60 (m, 1H), 7.58- 7.45 (m, 1H), 6.77 (t, J = 7.2 Hz, 1H), 6.67- 6.54 (m, 1H), 5.90 (dd, J = 44.5, 8.3 Hz, 1H), 5.02-4.89 (m, 1H), 4.86-4.76 (m, 1H), 4.54-4.43 (m, 1H), 4.33-4.10 (m, 2H), 4.10-4.00 (m, 1H), 3.90-3.82 (m, 1H), 3.63-3.51 (m, 6H), 3.46-3.33 (m, 2H), 2.35-2.25 (m, 1H), 2.22-2.03 (m, 1H), 1.90-1.67 (m, 2H) |
| 394 | ((6-(((5S,8S,10aR)- 3-(5-methylbenzo [d]isoxazole-3- carbonyl)-8- (morpholine-4-carbonyl)- 6-oxodecahydropyrrolo [1,2-a][1,5]diazocin-5- yl)carbamoyl) isoquinolin-3-yl)methyl) phosphonic acid | 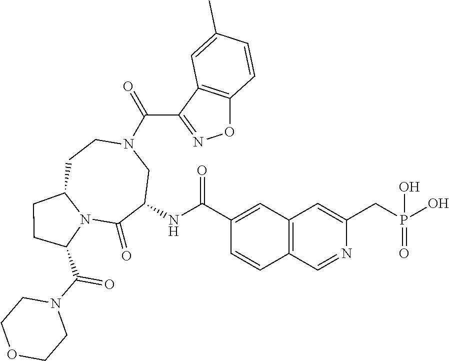 [SMILES: Cc1ccc2onc(C(=O)N3CC[C@H]4CC[C@@H](C(=O)N5CCOCC5)N4C(=O)[C@@H](NC(=O)c4ccc5cnc(CP(=O)(O)O)cc5c4)C3)c2c1] | 705.4 [M + H]⁺ | ¹H NMR (400 MHz, DMSO-d₆) δ 9.30- 9.11 (m, 1H), 9.01- 8.82 (m, 1H), 8.50- 8.30 (m, 1H), 8.13- 7.98 (m, 1H), 7.94- 7.89 (m, 1H), 7.86- 7.80 (m, 1H), 7.76- 7.64 (m, 1H), 7.57- 7.47 (m, 1H), 5.33- 5.11 (m, 1H), 4.90- 4.75 (m, 1H), 4.56- 4.55 (m, 1H), 4.12- 3.92 (m, 4H), 3.91- 3.82 (m, 2H), 3.34- 3.14 (m, 8H), 2.45- 2.40 (m, 3H), 2.30- 2.21 (m, 2H), 2.19- 2.10 (m, 1H), 1.96- 1.76 (m, 3H) |
| 395 | ((2-(((5S,8S,10aR)-8- ((3S,4R)-3- cyano-4-phenyl- pyrrolidine-1-carbonyl)- 3-(2,2-difluoroethyl)- 6-oxodecahydropyrrolo [1,2-a][1,5]diazocin-5- yl)carbamoyl)benzo[b] thiophen-5-yl) fluoromethyl) phosphonic acid | 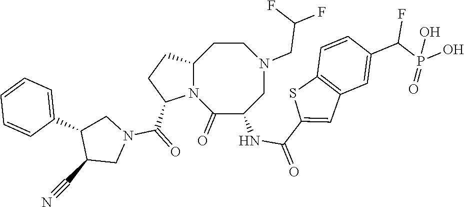 [SMILES: N#C[C@@H]1CN(C(=O)[C@@H]2CC[C@@H]3CCN(CC(F)F)C[C@H](NC(=O)c4cc5cc(C(F)P(=O)(O)O)ccc5s4)C(=O)N32)C[C@H]1c1ccccc1] | 718.1 [M + H]⁺ | ¹H NMR (400 MHz, DMSO-d₆) δ 8.62- 8.57 (m, 1H), 8.29- 8.23 (m, 1H), 8.08- 7.96 (m, 2H), 7.54 (d, J = 8.4 Hz, 1H), 7.49- 7.38 (m, 4H), 7.37- 7.32 (m, 1H), 6.12 (d, J = 3.6 Hz, 1H), 5.91- 5.78 (m, 1H), 4.95- 4.83 (m, 1H), 4.65- 4.53 (m, 1H), 4.41- 4.27 (m, 2H), 4.16- |
| | | | | 4.10 (m, 1H), 3.98 (s, |
| | | | | 1H), 3.84-3.82 (m, |
| | | | | 1H), 3.74 (s, 1H), |
| | | | | 3.67-3.67 (m, 1H), |
| | | | | 3.56 (s, 1H), 3.32- |
| | | | | 3.26 (m, 1H), 3.18 |
| | | | | (s, 1H), 3.15 (s, 1H), |
| | | | | 3.11 (s, 1H), 3.07 |
| | | | | (d, J = 2.4 Hz, 2H), |
| | | | | 2.33 (s, 1H), 2.10- |
| | | | | 2.02 (m, 1H), 1.84 (d, |
| | | | | J = 11.2 Hz, 1H), 1.80- |
| | | | | 1.66 (m, 3H) |
| 396 | ((7-(((5S,8S,10aR)-3- (5-methylbenzo[d] isoxazole-3-carbonyl)- 8-(morpholine-4- carbonyl)-6- oxodecahydropyrrolo [1,2-a][1,5]diazocin-5- yl)carbamoyl)quinolin- 2-yl)methyl)phosphonic acid | 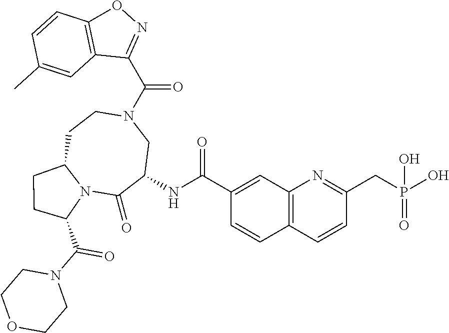 [SMILES: Cc1ccc2onc(C(=O)N3CC[C@H]4CC[C@@H](C(=O)N5CCOCC5)N4C(=O)[C@@H](NC(=O)c4ccc5ccc(CP(=O)(O)O)nc5c4)C3)c2c1] | 705.3 [M + H]⁺ | ¹H NMR (400 MHz, METHANOL-d₄) δ 8.97-8.67 (m, 1H), 8.52-7.82 (m, 5H), 7.75-7.44 (m, 2H), 5.49-5.31 (m, 3H), 4.65-4.55 (m, 1H), 4.42-4.02 (m, 4H), 3.89-3.48 (m, 9H), 2.65-2.17 (m, 7H), 2.12-1.73 (m, 3H) |
| 397 | ((7-(((5S,8S,10aR)-8- ((2S,6R)-2,6- dimethylmorpholine-4- carbonyl)-3-(5- methylbenzo[d] isoxazole-3-carbonyl)-6- oxodecahydropyrrolo [1,2-a][1,5]diazocin-5- yl)carbamoyl) naphthalen-2-yl) fluoromethyl)phosphonic acid | 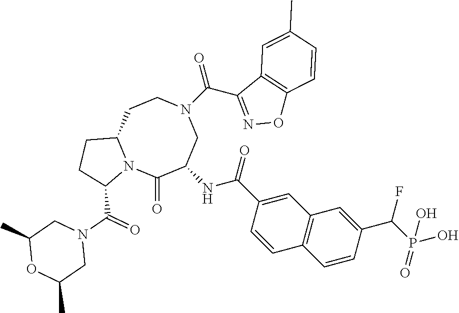 [SMILES: Cc1ccc2onc(C(=O)N3CC[C@H]4CC[C@@H](C(=O)N5C[C@@H](C)O[C@@H](C)C5)N4C(=O)[C@@H](NC(=O)c4ccc5ccc(C(F)P(=O)(O)O)cc5c4)C3)c2c1] | 750.4 [M + H]⁺ | ¹H NMR (400 MHz, DMSO-d₆) δ 8.96- 8.78 (m, 1H), 8.77- 8.73 (m, 1H), 7.98- 7.93 (m, 5H), 7.71- 7.67 (m, 2H), 7.67- 7.50 (m, 1H), 5.92- 5.77 (m, 1H), 5.30- 4.89 (m, 1H), 4.89- 4.48 (m, 2H), 4.18 (dd, J = 8.8, 10.0 Hz, 1H), 3.97-3.83 (m, 2H), 3.82-3.80 (m, 2H), 3.80-3.79 (m, 2H), 3.18-3.12 (m, 2H), 2.68-2.65 (m, 1H), 2.50-2.44 (m, 3H), 2.27-2.22 (m, 2H), 2.12-2.02 (m, 1H), 1.85-1.78 (m, 2H), |
| | | | | 1.25-1.07 (m, 1H), |
| | | | | 1.07-0.92 (m, 6H) |
| 398 | ((2-(((5S,8S,10aR)-8- (3-(6-cyanopyridin-2- yl)azetidine-1-carbonyl)- 6-oxo-3-(pyrimidin-2- ylmethyl) decahydropyrrolo[1,2- a][1,5]diazocin-5-yl) carbamoyl)benzo[b] thiophen-5-yl) fluoromethyl)phosphonic acid | 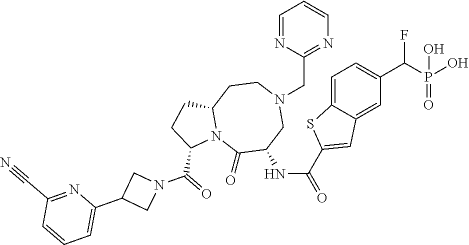 [SMILES: N#Cc1cccc(C2CN(C(=O)[C@@H]3CC[C@@H]4CCN(Cc5ncccn5)C[C@H](NC(=O)c5cc6cc(C(F)P(=O)(O)O)ccc6s5)C(=O)N43)C2)n1] | 733.1 [M + H]⁺ | ¹H NMR (400 MHz, DMSO-d₆) δ 8.80- 8.74 (m, 2H), 8.59 (t, J = 7.2 Hz, 1H), 8.23 (d, J = 14.8 Hz, 1H), 8.09-7.89 (m, 4H), 7.74 (t, J = 7.2 Hz, 1H), 7.50 (d, J = 6.0 Hz, 1H), 7.39 (m, 1H), 5.80-5.59 (m, 1H), 4.98-4.88 (m, 1H), 4.62-4.49 (m, 1H), 4.46-4.32 (m, 3H), 4.31-4.24 (m, 1H), 4.23-4.03 (m, 5H), |
| | | | | 4.00-3.93 (m, 1H), |
| | | | | 3.04-2.98 (m, 2H), |
| | | | | 2.33 (s, 1H), 2.27- |
| | | | | 2.14 (m, 1H), 2.14- |
| | | | | 1.99 (m, 1H), 1.95- |
| | | | | 1.82 (m, 1H), 1.81- |
| | | | | 1.72 (m, 1H), 1.71- |
| | | | | 1.60 (m, 2H) |
| 399 | ((2-(((5S,8S,10aR)-8- (3-(6-cyanopyridin-2- yl)azetidine-1-carbonyl)- 6-oxo-3-(pyrimidin-2- ylmethyl)decahydro- pyrrolo[1,2-a][1,5] diazocin-5-yl) carbamoyl)benzo[b] thiophen-5-yl)methyl) phosphonic acid | 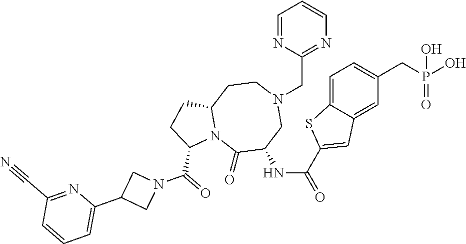 [SMILES: N#Cc1cccc(C2CN(C(=O)[C@@H]3CC[C@@H]4CCN(Cc5ncccn5)C[C@H](NC(=O)c5cc6cc(CP(=O)(O)O)ccc6s5)C(=O)N43)C2)n1] | 715.4 [M + H]⁺ | ¹H NMR (400 MHz, METHANOL-d₄) δ 8.85-8.76 (m, 2H), 8.03-7.90 (m, 2H), 7.85 (d, J = 7.2 Hz, 1H), 7.82-7.74 (m, 2H), 7.73-7.57 (m, 1H), 7.50-7.36 (m, 2H), 5.33-5.19 (m, 1H), 4.74-4.58 (m, 3H), 4.55-4.37 (m, 3H), 4.36-4.26 (m, 1H), 4.25-4.09 (m, 2H), 3.70-3.38 (m, 4H), 3.21-3.08 (m, 2H), 2.49-2.36 (m, |
| | | | | 1H), 2.32-2.18 (m, |
| | | | | 1H), 2.17-2.01 (m, |
| | | | | 2H), 1.97-1.84 (m, |
| | | | | 2H) |
| 400 | ((2-(((5S,8S,10aR)-8- (((3S,4S)-1-acetyl-4- fluoropyrrolidin-3- yl)carbamoyl)-6-oxo-3- (pyrimidin-2-ylmethyl) decahydropyrrolo[1,2- a][1,5]diazocin-5-yl) carbamoyl)benzo[b] thiophen-5-yl) fluoromethyl)phosphonic acid | 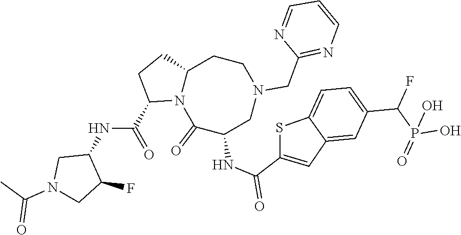 [SMILES: CC(=O)N1C[C@H](NC(=O)[C@@H]2CC[C@@H]3CCN(Cc4ncccn4)C[C@H](NC(=O)c4cc5cc(C(F)P(=O)(O)O)ccc5s4)C(=O)N32)[C@@H](F)C1] | 720.3 [M + H]⁺ | ¹H NMR (400 MHz, METHANOL-d₄) δ 8.79 (t, J = 5.2 Hz, 2H), 8.08-8.00 (m, 2H), 7.86 (d, J = 8.4 Hz, 1H), 7.63 (d, J = 3.6 Hz, 1H), 7.40 (q, J = 5.2 Hz, 1H), 5.75- 5.55 (m, 1H), 5.27- 4.97 (m, 3H), 4.57- 4.38 (m, 3H), 4.27- 4.06 (m, 2H), 3.94- 3.63 (m, 4H), 3.22- 3.03 (m, 3H), 2.34- 2.18 (m, 2H), 2.13- |
| | | | | 1.97 (m, 4H), 1.94- |
| | | | | 1.79 (m, 2H), 1.75- |
| | | | | 1.63 (m, 1H) |
| 401 | (difluoro(2-(((5S,8S, 10aR)-6-oxo-8-(3- (pyridin-3-yl)azetidine- 1-carbonyl)-3- ((quinuclidin-4- ylmethyl)carbamoyl) decahydropyrrolo [1,2-a][1,5]diazocin-5- yl)carbamoyl)benzo[b] thiophen-5-yl)methyl) phosphonic acid | 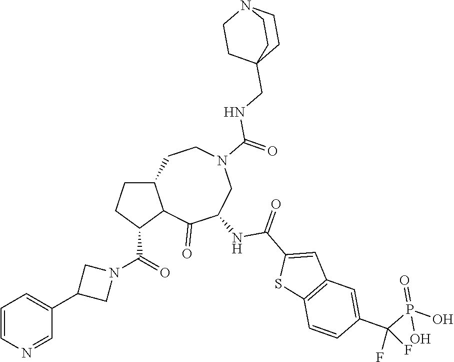 | 800 [M + H]⁺ | ¹H NMR (400 MHz, METHANOL-d₄) δ 8.86 (s, 1H) 8.69 (s, 2H) 8.19 (s, 1H) 8.06- 8.10 (m, 1H) 7.97- 8.03 (m, 1H) 7.91- 7.95 (m, 1H) 7.71- 7.75 (m, 1H) 4.63- 4.70 (m, 1H) 4.59 (m, 1H) 4.46-4.54 (m, 1H) 4.20-4.38 (m, 2H) 4.01-4.19 (m, 5H) 3.34-3.35 (m, 2H) 3.19 (s, 2H) 1.99-2.43 (m, 7H) 1.69-1.95 (m, 11H) |
| 402 | ((2-(((5S,8S,10aR)-3- (2-(1H-1,2,4-triazol-1- yl)benzyl)-6-oxo-8-(3- (pyridin-3-yl)azetidine- 1-carbonyl)decahydro- pyrrolo[1,2-a][1,5] diazocin-5-yl) carbamoyl)benzo[b] thiophen-5-yl) difluoromethyl) phosphonic acid | 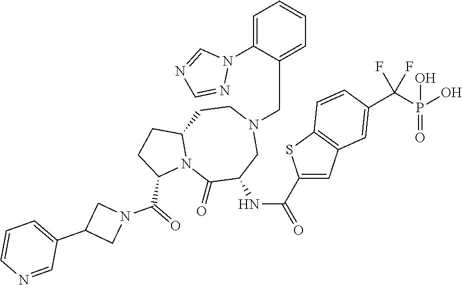 [SMILES: O=C(N[C@H]1CN(Cc2ccccc2-n2cncn2)CC[C@H]2CC[C@@H](C(=O)N3CC(c4cccnc4)C3)N2C1=O)c1cc2cc(C(F)(F)P(=O)(O)O)ccc2s1] | 791.1 [M + H]⁺ | ¹H NMR (400 MHz, METHANOL-d₄) δ 8.90 (s, 1H), 8.70-8.58 (m, 1H), 8.50 (d, J = 5.2 Hz, 1H), 8.29-8.25 (m, 1H), 8.19 (s, 1H), 8.09 (s, 1H), 8.03-7.95 (m, 1H), 7.94-7.88 (m, 1H), 7.76 (d, J = 8.4 Hz, 2H), 7.61-7.47 (m, 4H), 5.09-4.93 (m, 2H), 4.75-4.66 (m, 1H), 4.61-4.50 (m, 1H), 4.46-4.27 (m, 2H), 4.12-3.97 (m, 3H), 3.95-3.78 (m, 1H), 3.44-3.37 (m, 1H), 3.23-2.73 (m, |
| | | | | 3H), 2.48-2.15 (m, |
| | | | | 2H), 2.09-2.00 (m, |
| | | | | 1H), 1.93-1.66 (m, |
| | | | | 2H), 1.64-1.32 (m, |
| | | | | 1H) |
| 403 | ((2-(((5S,8S,10aR)-3- (2-chloro-4-(4- methylpiperazin-1- yl)benzoyl)-6-oxo-8- (3-(pyridin-3-yl) azetidine-1-carbonyl) decahydropyrrolo[1,2- a][1,5]diazocin-5- yl)carbamoyl)benzo[b] thiophen-5-yl) difluoromethyl) phophonic acid | 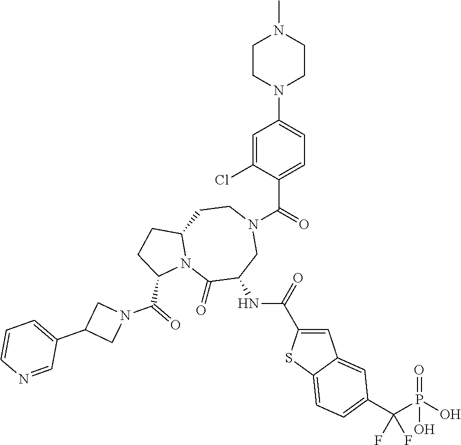 [SMILES: CN1CCN(c2ccc(C(=O)N3CC[C@H]4CC[C@@H](C(=O)N5CC(c6cccnc6)C5)N4C(=O)[C@@H](NC(=O)c4cc5cc(C(F)(F)P(=O)(O)O)ccc5s4)C3)c(Cl)c2)CC1] | 870.3 [M + H]⁺ | ¹H NMR (400 MHz, METHANOL-d₄) δ 8.71-8.55 (m, 1H), 8.51-8.43 (m, 1H), 8.23-8.15 (m, 1H), 8.07-7.90 (m, 3H), 7.73 (d, J = 9.2 Hz, 1H), 7.54-7.48 (m, 1H), 7.45-7.37 (m, 1H), 7.03 (s, 1H), 6.80 (d, J = 6.0 Hz, 1H), 5.35-5.16 (m, 1H), 5.05-4.96 (m, 1H), 4.73 (d, J = 7.2 Hz, 1H), 4.62-4.46 (m, 3H), 4.38-4.25 (m, 1H), 4.13-3.97 (m, 3H), 3.79-3.64 (m, 2H), 3.55-3.46 (m, 1H), 3.28 (s, 2H), 3.11-2.88 (m, 5H), 2.69 (d, J = 2.4 Hz, 3H), 2.50-2.27 (m, 3H), 2.17-1.94 (m, 3H) |
| 404 | ((2-(((5R,8S,10aR)- 3,3-dioxido-6-oxo-8-(3- (pyridin-3-yl)azetidine- 1-carbonyl)octahydro- 4H-pyrrolo[2,1-d][1,5] thiazocin-5-yl) carbamoyl)benzo[b] thiophen-5-yl) difluoromethyl) phosphonic acid | 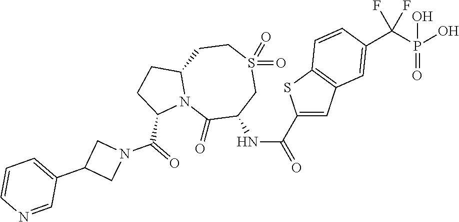 [SMILES: O=C(N[C@H]1CS(=O)(=O)CC[C@H]2CC[C@@H](C(=O)N3CC(c4cccnc4)C3)N2C1=O)c1cc2cc(C(F)(F)P(=O)(O)O)ccc2s1] | 683 [M + H]⁺ | ¹H NMR (400 MHz, METHANOL-d₄) δ 8.56 (m, 1H), 8.49- 8.44 (m, 1H), 8.20 (s, 1H), 8.12 (d, J = 2.0 Hz, 1H), 8.08-7.90 (m, 2H), 7.75 (d, J = 8.4 Hz, 1H), 7.46 (m, 1H), 5.31 (m, 1H), 5.00-4.94 (m, 1H), 4.75-4.61 (m, 2H), 4.60-4.29 (m, 2H), 4.12-3.95 (m, 3H), 3.78 (m, 1H), 3.50 (d, |
| | | | | J = 14.8 Hz, 1H), 3.34 |
| | | | | (d, J = 2.4 Hz, 1H), |
| | | | | 2.47-2.34 (m, 1H), |
| | | | | 2.29-2.18 (m, 2H), |
| | | | | 2.11-1.91 (m, 3H) |
| 405 | (difluoro(2-(((5R,8S, 10aR)-6-oxo-8-(3- (pyridin-3-yl)azetidine-1- carbonyl)octahydro-4H- pyrrolo[2,1-d][1,5] thiazocin-5-yl) carbamoyl)benzo[b] thiophen-5-yl)methyl) phosphonic acid | 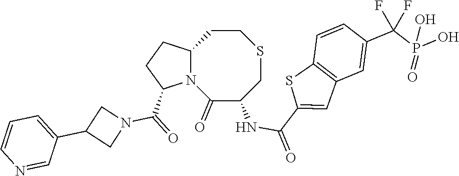 [SMILES: O=C(N[C@H]1CSCC[C@H]2CC[C@@H](C(=O)N3CC(c4cccnc4)C3)N2C1=O)c1cc2cc(C(F)(F)P(=O)(O)O)ccc2s1] | 651.1 [M + H]⁺ | ¹H NMR (400 MHz, METHANOL-d₄) δ 8.66-8.65 (m, 1H), 8.53-8.45 (m, 1H), 8.24-8.16 (m, 2H), 8.12-8.09 (m, 1H), 7.98-7.90 (m, 1H), 7.77-7.71 (m, 1H), 7.61-7.55 (m, 1H), 5.05-5.05 (m, 1H), 5.07-5.02 (m, 1H), |
| | | | | 4.71 (d, J = 8.4 Hz, |
| | | | | 2H), 4.58-4.52 (m, |
| | | | | 1H), 4.10-3.94 (m, |
| | | | | 2H), 3.06-2.81 (m, |
| | | | | 4H), 2.42-2.30 (m, |
| | | | | 1H), 2.25-2.15 (m, |
| | | | | 2H), 2.13-1.99 (m, |
| | | | | 1H), 1.95-1.73 (m, |
| | | | | 2H), 1.39-1.34 (m, |
| | | | | 1H) |

### Phosphonate Esters and Amides Data Tables:

The following compounds in Table 28 were prepared according to the representative procedure described above for the synthesis of S,S′-(((((7-(((5S,8S,10aR)-3-acetyl-6-oxo-8-(6-phenyl-4-azaspiro[2.4]heptane-4-carbonyl)decahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate) (1) utilizing the appropriate starting materials and modifications.

TABLE 28

| Com- | Name | Structure | LCMS | NMR |
|---|---|---|---|---|
| pound | | | | |
| 406 | propyl (((7- (((5S,8S,10aR)- 3-acetyl- 8-(6-(1- methyl-2- oxo-1,2- dihydro- pyridin- 4-yl)-4- azaspiro[2.4] heptane-4- carbonyl)-6- oxodeca- hydropyrrolo [1,2-a] [1,5]diazocin- 5-yl)carbamoyl) naphthalen- 2-yl)fluoromethyl) (phenoxy) phosphoryl)- L-alaninate | 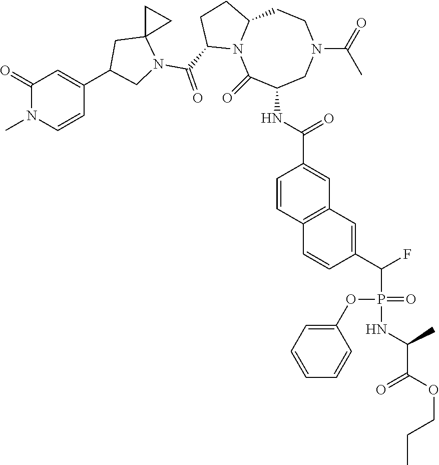 [SMILES: CCCOC(=O)[C@H](C)NP(=O)(Oc1ccccc1)C(F)c1ccc2ccc(C(=O)N[C@H]3CN(C(C)=O)CC[C@H]4CC[C@@H](C(=O)N5CC(c6ccn(C)c(=O)c6)CC56CC6)N4C3=O)cc2c1] | 911.4 [M + H]⁺ | ¹H NMR (400 MHZ, CDCl₃) δ 8.44-8.35 (m, 1H), 8.11-8.01 (m, 1H), 7.98-7.81 (m, 4H), 7.79-7.69 (m, 1H), 7.39-7.32 (m, 1H), 7.31-7.23 (m, 4H), 7.23-7.17 (m, 1H), 7.10-7.06 (m, 1H), 6.67-6.45 (m, 1H), 6.26-5.91 (m, 2H), 4.94-4.78 (m, 1H), 4.60-4.40 (m, 1H), 4.23-3.92 (m, 6H), 3.88-3.58 (m, 2H), 3.52 (s, 3H), 3.49-3.18 (m, 3H), 2.50 (s, 3H), 2.35- 2.16 (m, 4H), 2.09- 1.98 (m, 3H), 1.83- 1.69 (m, 2H), 1.64- 1.42 (m, 2H), 1.36- 0.75 (m, 7H), 0.65- 0.46 (m, 2H) |
| 407 | propyl (((7- (((5S,8S,10aR)- 3-(8- chloro-2- methylquinoline- 6-carbonyl)-8- (morpholine- 4-carbonyl)-6- oxodeca- hydropyrrolo [1,2-a][1,5] diazocin-5- yl)carbamoyl) naphthalen- 2-yl) fluoromethyl) (phenoxy) phosphoryl)- L-alaninate | 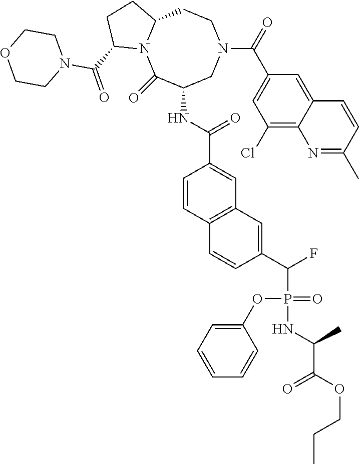 [SMILES: CCCOC(=O)[C@H](C)NP(=O)(Oc1ccccc1)C(F)c1ccc2ccc(C(=O)N[C@H]3CN(C(=O)c4cc(Cl)c5nc(C)ccc5c4)CC[C@H]4CC[C@@H](C(=O)N5CCOCC5)N4C3=O)cc2c1] | 955.3 [M + H]⁺ | ¹H NMR (400 MHZ, DMSO-d₆) δ 8.73- 8.58 (m, 1H), 8.45- 8.26 (m, 2H), 8.18- 8.10 (m, 1H), 8.09- 7.86 (m, 5H), 7.77- 7.55 (m, 2H), 7.39- 7.28 (m, 2H), 7.22- 7.07 (m, 3H), 6.35- 6.07 (m, 2H), 5.44- 5.32 (m, 1H), 4.92- 4.79 (m, 1H), 4.64- 4.52 (m, 1H), 4.20- 4.04 (m, 1H), 3.96- 3.73 (m, 4H), 3.70- 3.51 (m, 10H), 2.71 (s, 3H), 2.41-2.10 (m, 3H), 1.90-1.69 (m, 3H), 1.52-1.32 (m, 2H), 1.18-0.91 (m, 3H), 0.85-0.69 (m, 3H) |
| 408 | propyl (((7- (((5S,8S,10aR)- 3-acetyl- 8-(6-(1- methyl-2- oxo-1,2- dihydropyridin- 4-yl)-4- azaspiro [2.4] heptane-4- carbonyl)-6- oxodeca- hydropyrrolo [1,2-a] [1,5]diazocin- 5-yl)carbamoyl) naphthalen- 2-yl)methyl) (phenoxy) phosphoryl)-L- alaninate | 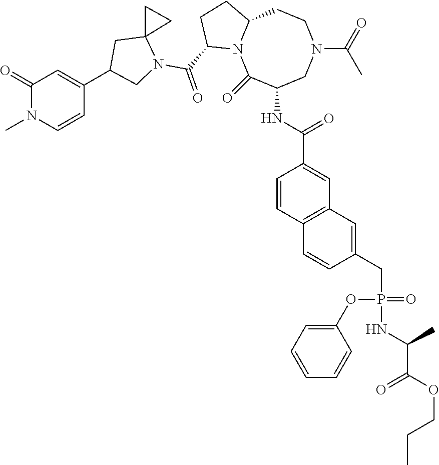 [SMILES: CCCOC(=O)[C@H](C)NP(=O)(Cc1ccc2ccc(C(=O)N[C@H]3CN(C(C)=O)CC[C@H]4CC[C@@H](C(=O)N5CC(c6ccn(C)c(=O)c6)CC56CC6)N4C3=O)cc2c1)Oc1ccccc1] | 893.6 [M + H]⁺ | ¹H NMR (400 MHZ, CDCl₃) δ 8.39-8.29 (m, 1H), 7.97-7.80 (m, 5H), 7.67-7.54 (m, 1H), 7.32-7.27 (m, 2H), 7.26-7.24 (m, 1H), 7.19-7.07 (m, 3H), 6.59-6.49 (m, 1H), 6.21-6.12 (m,, 1H), 4.92-4.81 (m, 1H), 4.63-4.39 (m, 1H), 4.23-4.07 (m, 3H), 4.06-3.85 (m, 4H), 3.58-3.45 (m, 6H), 3.44-3.11 (m, 4H), 2.49 (s, 3H), 2.38- 2.18 (m, 4H), 2.07- 1.87 (m, 6H), 1.62- 1.47 (m, 2H), 1.22- 1.09 (m, 3H), 0.92- 0.78 (m, 3H), 0.65- 0.45 (m, 2H) |
| 409 | propyl (((7- (((5S,8S,10aR)- 3-acetyl- 6-oxo-8-(3- (pyridin-3- yl)pyrrolidine- 1-carbonyl) decahydropyrrolo [1,2-a] [1,5]diazocin-5- yl)carbamoyl) naphthalen- 2- yl)fluoromethyl) (phenoxy) phosphoryl)- L-alaninate | 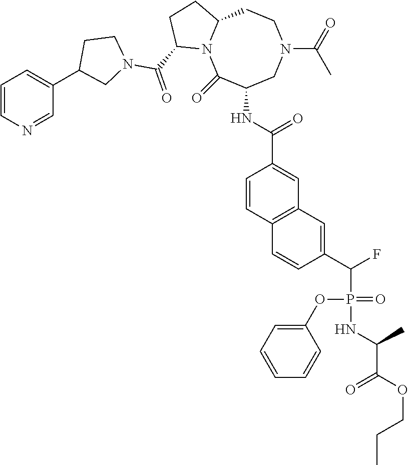 [SMILES: CCCOC(=O)[C@H](C)NP(=O)(Oc1ccccc1)C(F)c1ccc2ccc(C(=O)N[C@H]3CN(C(C)=O)CC[C@H]4CC[C@@H](C(=O)N5CCC(c6cccnc6)C5)N4C3=O)cc2c1] | 855.2 [M + H]⁺ | ¹H NMR (400 MHZ, DMSO-d6) δ 8.84- 8.42 (m, 4H), 8.21- 7.71 (m, 6H), 7.50- 7.07 (m, 6H), 6.42- 6.08 (m, 2H), 5.09- 4.87 (m, 1H), 4.70- 4.58 (m, 1H), 4.38- 4.22 (m, 1H), 4.10- 3.63 (m, 7H), 3.63- 3.43 (m, 3H), 3.37- 3.30 (m, 2H), 2.43- 1.69 (m, 11H), 1.54- 1.36 (m, 2H), 1.22- 0.93 (m, 3H), 0.88- 0.64 (m, 3H) |
| 410 | propyl ((fluoro(7- (((5S,8S,10aR)- 3-(5- methylbenzo [d]isoxazole- 3-carbonyl)- 8-(4- (methylsulfonyl) piperazine- 1-carbonyl)-6- oxodeca- hydropyrrolo [1,2-a][1,5] diazocin-5- yl)carbamoyl) naphthalen- 2-yl)methyl) (phenoxy) phosphoryl)-L- alaninate | 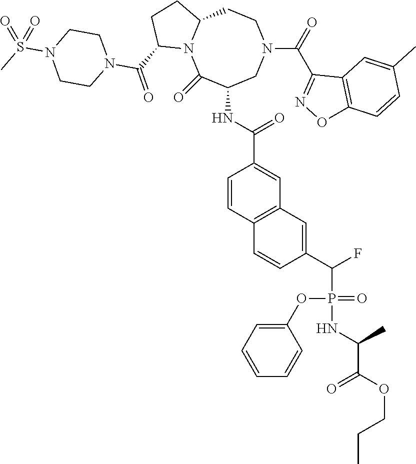 | 988.5 [M + H]⁺ | ¹H NMR (400 MHZ, CDCl₃) δ 8.47-8.28 (m, 1H), 8.06-7.28 (m, 10H), 7.25-7.02 (m, 3H), 6.13-5.91 (m, 1H), 5.49-5.27 (m, 1H), 4.90-4.70 (m, 1H), 4.55-4.34 (m, 2H), 4.29-3.55 (m, 9H), 3.54-3.01 (m, 5H), 2.77-2.64 (m, 3H), 2.47 (s, 3H), 2.29-2.18 (m, 2H), 2.09-1.94 (m, 2H), 1.90-1.82 (m, 2H), 1.66-1.45 (m, 2H), 1.33-1.06 (m, 3H), 0.95-0.75 (m, 3H) (TFA salt) |
| 411 | propyl (((7- (((5S,8S,10aR)- 8-(4-(benzo[d] isothiazol-3- yl)piperazine-1- carbonyl)- 3-(5- methylbenzo [d]isoxazole- 3-carbonyl)-6- oxodeca- hydropyrrolo [1,2-a][1,5] diazocin-5- yl)carbamoyl) naphthalen- 2-yl) fluoromethyl) (phenoxy) phosphoryl)- L-alaninate | 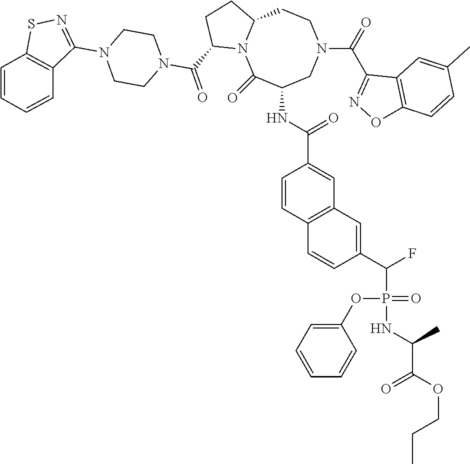 [SMILES: CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N5CCN(c6nsc7ccccc67)CC5)N4C3=O)cc2c1] | 1043.6 [M + H]⁺ | ¹H NMR (400 MHZ, CDCl₃) δ 8.46-8.26 (m, 1H), 8.08-7.29 (m, 14H), 7.25-7.16 (m, 2H), 7.09-7.04 (m, 2H), 6.13-5.89 (m, 1H), 5.42-5.27 (m, 1H), 4.96-4.89 (m, 1H), 4.71-4.40 (m, 2H), 4.30-3.36 (m, 15H), 2.51-2.44 (m, 3H), 2.41-2.25 (m, 2H), 2.24-2.11 (m, 2H), 1.97-1.86 (m, 2H), 1.68-1.51 (m, 2H), 1.34-1.19 (m, 3H), 0.95-0.78 (m, 3H) |
| 412 | propyl ((fluoro(7- (((5S,8S,10aR)- 3-(5- methylbenzo [d]isoxazole- 3-carbonyl)- 6-oxo-8-((S)-3- phenyl- morpholine- 4-carbonyl) decahydro- pyrrolo [1,2-a][1,5] diazocin-5- yl)carbamoyl )naphthalen- 2- yl)methyl) (phenoxy) phosphoryl)-L- alaninate | 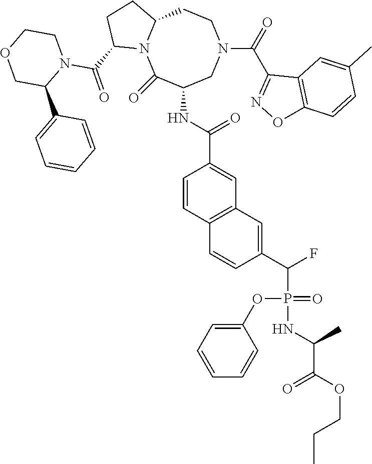 [SMILES: CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N5CCOC[C@@H]5c5ccccc5)N4C3=O)cc2c1] | 987.3 [M + H]⁺ | ¹H NMR (400 MHZ, DMSO-d₆) δ 9.20- 8.82 (m, 1H), 8.71- 8.48 (m, 1H), 8.22- 7.10 (m, 18H), 6.40- 6.09 (m, 2H), 5.56- 5.18 (m, 2H), 4.99- 4.71 (m, 1H), 4.64- 3.39 (m, 13H), 3.18- 2.85 (m, 1H), 2.48- 2.13 (m, 5H), 2.10- 1.33 (m, 6H), 1.28- 0.94 (m, 3H), 0.86- 0.71 (m, 3H) |
| 413 | propyl ((fluoro(7- (((5S,8S,10aR)- 3-(5- methylbenzo [d]isoxazole- 3-carbonyl)- 6-oxo-8- ((R)-3- phenyl- morpholine- 4-carbonyl) decahydro- pyrrolo [1,2-a][1,5] diazocin-5- yl)carbamoyl) naphthalen- 2-yl)methyl) (phenoxy) phosphoryl)-L- alaninate | 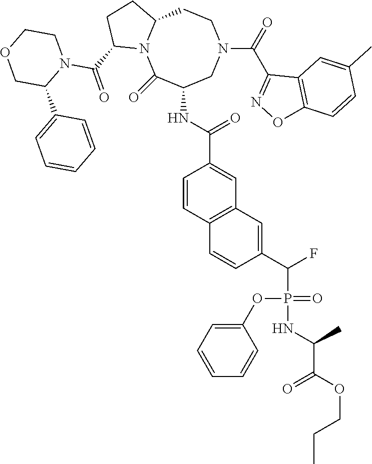 [SMILES: CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N5CCOC[C@H]5c5ccccc5)N4C3=O)cc2c1] | 987.4 [M + H]⁺ | ¹H NMR (400 MHZ, DMSO-d₆) δ 9.13- 8.66 (m, 1H), 8.65- 8.42 (m, 1H), 8.28- 7.85 (m, 5H), 7.84- 7.66 (m, 2H), 7.65- 6.94 (m, 11H), 6.45- 6.03 (m, 2H), 5.54- 3.61 (m, 16H), 3.30- 3.07 (m, 1H), 2.42- 2.27 (m, 3H), 2.26- 1.32 (m, 8H), 1.18- 1.06 (m, 2H), 1.03- 0.94 (m, 1H), 0.84- 0.71 (m, 3H) |
| 414 | propyl (((7- (((5S,8S,10aR)- 8-((1S,4S)-2- oxa-5- azabicyclo [2.2.1]heptane- 5-carbonyl)- 3-(5- methylbenzo [d]isoxazole- 3-carbonyl)- 6-oxodeca- hydropyrrolo [1,2-a][1,5] diazocin-5- yl)carbamoyl) naphthalen- 2-yl) fluoromethyl) (phenoxy) phosphoryl)-L- alaninate | 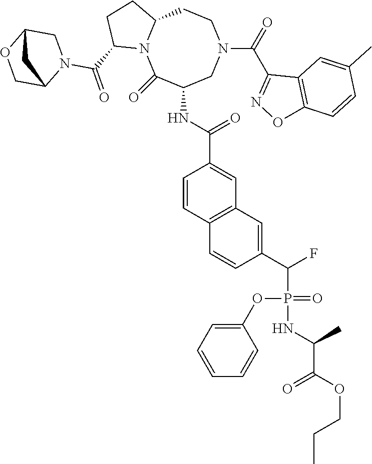 [SMILES: CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N5C[C@@H]6C[C@H]5CO6)N4C3=O)cc2c1] | 923.3 [M + H]⁺ | ¹H NMR (400 MHZ, CDCl₃) δ 8.45-8.22 (m, 1H), 8.07-7.68 (m, 6H), 7.62-7.31 (m, 3H), 7.26-7.01 (m, 5H), 6.15-5.89 (m, 1H), 5.40-5.20 (m, 1H), 4.99-4.78 (m, 1H), 4.73-4.55 (m, 2H), 4.54-4.38 (m, 2H), 4.31-4.19 (m, 1H), 4.18-3.27 (m, 10H), 2.53-2.44 (m, 3H), 2.39-1.94 (m, 6H), 1.88-1.80 (m, 2H), 1.65-1.53 (m, 2H), 1.35-1.25 (m, 3H), 0.95-0.85 (m, 3H) |
| 415 | propyl (((7- (((5S,8S,10aR)- 8-(2- benzyl- morpholine- 4-carbonyl)-3- (5-methylbenzo [d]isoxazole- 3-carbonyl)-6- oxodeca- hydropyrrolo [1,2-a][1,5] diazocin-5- yl)carbamoyl) naphthalen- 2-yl) fluoromethyl) (phenoxy) phosphoryl)- L-alaninate | 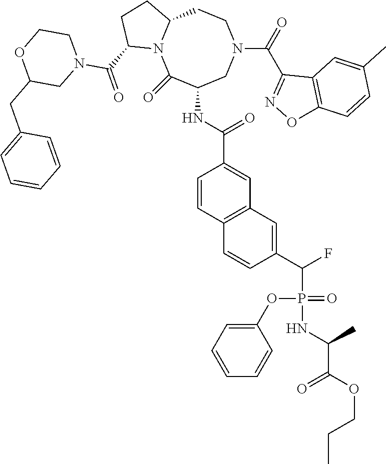 [SMILES: CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N5CCOC(Cc6ccccc6)C5)N4C3=O)cc2c1] | 1001.2 [M + H]⁺ | ¹H NMR (400 MHZ, DMSO-d₆) δ 9.03- 8.66 (m, 1H), 8.64- 8.44 (m, 1H), 8.19- 7.84 (m, 5H), 7.81- 7.65 (m, 2H), 7.58- 7.47 (m, 1H), 7.39- 7.07 (m, 10H), 6.44- 6.11 (m, 2H), 5.32- 5.09 (m, 1H), 4.92- 4.62 (m, 1H), 4.53- 3.67 (m, 11H), 3.62- 3.41 (m, 1H), 3.21- 2.54 (m, 4H), 2.48- 2.37 (m, 4H), 2.35- 1.88 (m, 4H), 1.86- 1.61 (m, 2H), 1.54- 1.33 (m, 2H), 1.21- 1.08 (m, 2H), 1.00- 0.92 (m, 1H), 0.85- 0.71 (m, 3H) |
| 416 | propyl (((1R)-(7- (((5S,8S,10aR)- 8-(8-oxa-3- azabicyclo [3.2.1]octane-3- carbonyl)-3-(5- methylbenzo [d]isoxazole- 3-carbonyl)-6- oxodeca- hydropyrrolo [1,2-a][1,5] diazocin-5- yl)carbamoyl) naphthalen- 2-yl) fluoromethyl) (phenoxy) phosphoryl)-L- alaninate | 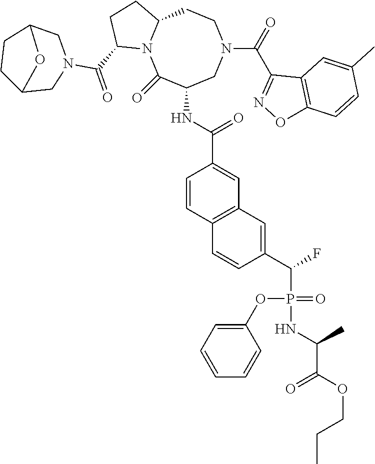 [SMILES: CCCOC(=O)[C@H](C)NP(=O)(Oc1ccccc1)[C@@H](F)c1ccc2ccc(C(=O)N[C@H]3CN(C(=O)c4noc5ccc(C)cc45)CC[C@H]4CC[C@@H](C(=O)N5CC6CCC(C5)O6)N4C3=O)cc2c1] | 937.3 [M + H]⁺ | ¹H NMR (400 MHZ, DMSO-d₆) δ 9.09- 8.57 (m, 1H), 8.54- 8.45 (m, 1H), 8.22- 8.08 (m, 1H), 8.08- 7.83 (m, 4H), 7.80- 7.62 (m, 2H), 7.58- 7.47 (m, 1H), 7.42- 7.28 (m, 2H), 7.25- 7.06 (m, 3H), 6.40- 6.27 (m, 1H), 6.27- 6.10 (m, 1H), 5.39- 5.11 (m, 1H), 4.96- 4.57 (m, 1H), 4.54- 4.39 (m, 1H), 4.35- 4.25 (m, 1H), 4.23- 4.13 (m, 1H), 4.12- 3.96 (m, 2H), 3.95- 3.83 (m, 3H), 3.81- 3.63 (m, 2H), 3.31- 3.14 (m, 2H), 2.86- 2.53 (m, 2H), 2.47- 2.42 (m, 3H), 2.37- 1.33 (m, 12H), 1.20- 1.04 (m, 3H), 0.85- 0.72 (m, 3H) |
| 417 | propyl (((S)- fluoro(7- (((5S,8S,10a R)-8-((R)-6- (1-methyl-2- oxo-1,2- dihydro- pyridin-4-yl)-4- azaspiro[2.4] heptane-4- carbonyl)-3-(5- methylbenzo [d]isoxazole- 3-carbonyl)-6- oxodeca- hydropyrrolo [1,2-a][1,5] diazocin-5- yl)carbamoyl) naphthalen-2- yl)methyl) (phenoxy) phosphoryl)-L- alaninate | 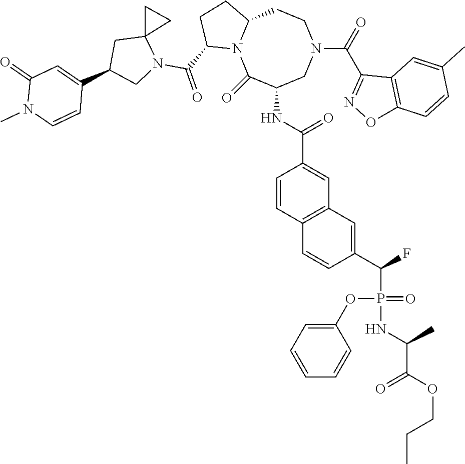 | 1028.5 [M + H]⁺ | ¹H NMR (400 MHZ, DMSO-d₆) δ 9.08- 8.75 (m, 1H), 8.68- 8.44 (m, 1H), 8.11- 7.45 (m, 9H), 6.34- 6.15 (m, 2H), 6.01- 5.83 (m, 1H), 5.33- 5.20 (m, 1H), 4.57- 4.40 (m, 2H), 4.31- 4.17 (m, 2H), 4.07- 3.86 (m, 3H), 3.57- 3.50 (m, 1H), 3.39- 3.32 (m, 3H), 3.28- 3.17 (m, 1H), 2.49- 2.44 (m, 3H), 2.35- 2.01 (m, 4H), 2.00- 1.52 (m, 6H), 0.52- 0.12 (m, 2H) (TFA salt) |
| 418 | propyl (((R)-(7- (((5S,8S,10aR)- 8-(5,7- dioxo-6- (pyridin-3- yl)-4,6- diazaspiro [2.4]heptane-4- carbonyl)-3- (5- methylbenzo [d]isoxazole- 3-carbonyl)-6- oxodeca- hydropyrrolo [1,2a][1,5] diazocin-5- yl)carbamoyl) naphthalen- 2-yl) fluoromethyl) (phenoxy) phosphoryl)-L- alaninate | 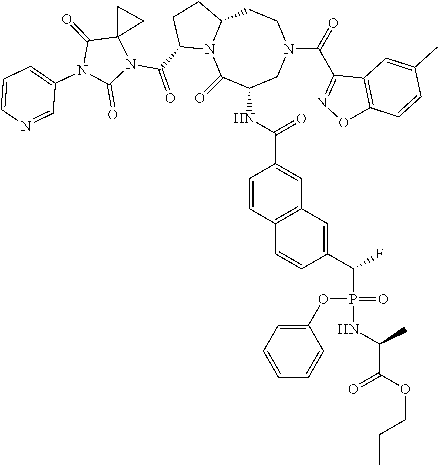 [SMILES: CCCOC(=O)[C@H](C)NP(=O)(Oc1ccccc1)[C@@H](F)c1ccc2ccc(C(=O)N[C@H]3CN(C(=O)c4noc5ccc(C)cc45)CC[C@H]4CC[C@@H](C(=O)N5C(=O)N(c6cccnc6)C(=O)C56CC6)N4C3=O)cc2c1] | 1027.4 [M + H]⁺ | ¹H NMR (400 MHZ, DMSO-d₆) δ 9.14- 8.48 (m, 4H), 8.19- 7.70 (m, 8H), 7.59- 7.52 (m, 2H), 7.39- 7.27 (m, 2H), 7.22- 7.09 (m, 3H), 6.36- 6.10 (m, 2H), 5.40- 5.29 (m, 2H), 4.63- 4.50 (m, 2H), 4.06- 3.58 (m, 6H), 2.46- 0.89 (m, 18H), 0.86-0.70 (m, 3H) |
| 419 | propyl (((S)- fluoro(7- (((5S,8S,10aR)- 8-((S)-6- (1-methyl-2- oxo-1,2- dihydropyridin- 4-yl)-4- azaspiro[2.4] heptane-4- carbonyl)-3-(5- methylbenzo [d]isoxazole- 3-carbonyl)-6- oxodeca- hydropyrrolo [1,2-a][1,5] diazocin-5- yl)carbamoyl) naphthalen- 2-yl)methyl) (phenoxy) phosphoryl)-L- alaninate | 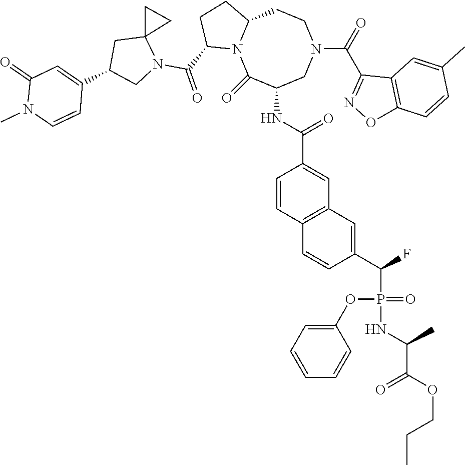 | 1028.1 [M + H]⁺ | ¹H NMR (400 MHZ, CDCl₃) δ 8.42-8.26 (m, 1H), 8.08-7.93 (m, 2H), 7.92-7.77 (m, 4H), 7.76-7.65 (m, 2H), 7.52-7.45 (m, 1H), 7.42-7.36 (m, 1H), 7.35-7.32 (m, 1H), 7.26-7.16 (m, 3H), 7.12-7.06 (m, 1H), 6.49 (s, 1H), 6.20-6.09 (m, 1H), 6.09-5.92 (m, 1H), 5.42-5.27 (m, 1H), 4.75-4.21 (m, 4H), 4.11-4.00 (m, 2H), 3.98-3.90 (m, 2H), 3.90-3.85 (m, 1H), 3.85-3.77 (m, 2H), 3.76-3.66 (m, 1H), 3.47 (s, 3H), 3.38-3.26 (m, 1H), 2.48 (s, 3H), 2.35-2.24 (m, 2H), 2.20-2.09 (m, 3H), 2.03-1.99 (m, 1H), 1.93-1.89 (m, 2H), 1.85-1.75 (m, 1H), 1.67-1.52 |
| | | | | (m, 2H), 1.49-1.41 |
| | | | | (m, 1H), 1.32-1.28 |
| | | | | (m, 1H), |
| | | | | 1.26 (d, J = 6.8 Hz, |
| | | | | 2H), 0.95-0.88 (m, |
| | | | | 1H), 0.84-0.75 (m, |
| | | | | 2H), 0.52-0.22 |
| | | | | (m, 2H) |
| 420 | propyl (((R)- fluoro(7- (((5S,8S,10aR)- 8-((R)-6- (1-methyl-2- oxo-1,2- dihydropyridin- 4-yl)-4- azaspiro[2.4] heptane-4- carbonyl)-3-(5- methylbenzo [d]isoxazole- 3-carbonyl)-6- oxodeca- hydropyrrolo [1,2-a][1,5] diazocin-5- yl)carbamoyl) naphthalen- 2- yl)methyl) (phenoxy) phosphoryl)-L- alaninate | 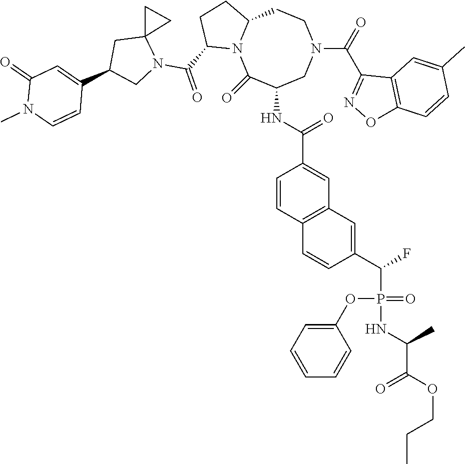 | 1028.6 [M + H]⁺ | ¹H NMR (400 MHZ, CDCl₃) δ 8.46-8.29 (m, 1H), 8.05-7.66 (m, 7H), 7.51-7.45 (m, 1H), 7.41-7.28 (m, 3H), 7.24-7.04 (m, 4H), 6.47-6.37 (m, 1H), 6.12-5.92 (m, 2H), 5.44-5.29 (m, 1H), 4.65-4.18 (m, 5H), 4.15-3.61 (m, 7H), 3.53-3.42 (m, 4H), 3.38-3.26 (m, 1H), 2.52-2.43 (m, 3H), 2.31-2.03 (m, 6H), 1.95-1.87 (m, 2H), 1.62-1.39 (m, 3H), 1.35-1.02 (m, 4H), 0.92-0.86 (m, 3H), 0.55-0.16 (m, 2H) |
| 421 | propyl (((R)- fluoro(7- (((5S,8S,10aR)- 8-((S)-6- (1-methyl-2- oxo-1,2- dihydropyridin- 4-yl)-4-azaspiro [2.4] heptane-4- carbonyl)-3-(5- methylbenzo [d]isoxazole- 3-carbonyl)-6- oxodeca- hydropyrrolo [1,2-a] [1,5]diazocin- 5-yl) carbamoyl) naphthalen- 2-yl)methyl) (phenoxy) phosphoryl)-L- alaninate | 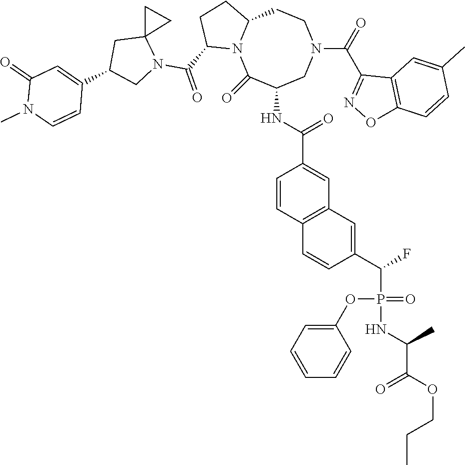 | 1028.1 [M + H]⁺ | ¹H NMR (400 MHZ, CDCl₃) δ 8.44-8.26 (m, 1H), 8.03-7.62 (m, 7H), 7.52-7.43 (m, 1H), 7.42-7.28 (m, 3H), 7.25-7.04 (m, 4H), 6.48 (s, 1H), 6.19-5.89 (m, 2H), 5.45-5.29 (m, 1H), 4.68-4.20 (m, 4H), 4.14-3.98 (m, 4H), 3.96-3.67 (m, 4H), 3.52-3.39 (m, 3H), 3.37-3.21 (m, 1H), 2.47 (s, 3H), 2.33- 2.16 (m, 4H), 2.06- 2.02 (m, 1H), 1.95- 1.79 (m, 3H), 1.66- 1.49 (m, 3H), 1.36- 1.20 (m, 2H), 1.18- 1.01 (m, 2H), 0.94- 0.84 (m, 3H), 0.58- 0.16 (m, 2H). |
| 422 | propyl (((7- (((5S,8S,10aR)- 2-(3-chloro-5- methylbenzo yl)-8- (morpholine- 4-carbonyl)-6- oxodeca- hydropyrrolo [1,2-a][1,4] diazocin-5- yl)carbamoyl) naphthalen-2- yl) fluoromethyl) (phenoxy) phosphoryl)- L-alaninate | 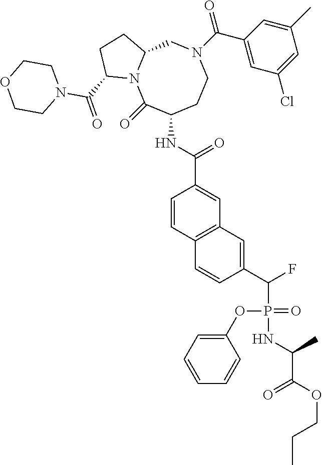 [SMILES: CCCOC(=O)[C@H](C)NP(=O)(Oc1ccccc1)C(F)c1ccc2ccc(C(=O)N[C@H]3CCN(C(=O)c4cc(C)cc(Cl)c4)C[C@H]4CC[C@@H](C(=O)N5CCOCC5)N4C3=O)cc2c1] | 904.3 [M + H]⁺ | ¹H NMR (400 MHZ, CDCl₃) δ 8.36-8.27 (m, 1H), 8.05-7.97 (m, 1H), 7.91-7.80 (m, 3H), 7.75-7.65 (m, 1H), 7.45-7.29 (m, 3H), 7.25-7.04 (m, 5H), 6.11-5.92 (m, 1H), 5.33-5.18 (m, 1H), 5.04-4.92 (m, 1H), 4.67-4.30 (m, 2H), 4.17-3.90 (m, 3H), 3.86-3.51 (m, 11H), 3.45-2.85 (m, 1H), 2.44-2.34 (m, 3H), 2.34-2.17 (m, 2H), 2.10-1.85 (m, 3H), 1.67-1.39 (m, 3H), 1.32-1.06 (m, 3H), 0.97-0.72 (m, 3H) |

The following compounds in Table 29 were prepared according to the representative procedure described above for the synthesis of (difluoro(2-(((S,8S,10aR)-8-(methyl(phenyl)carbamoyl)-6-oxo-3-(2-phenylacetyl)decahydropyrrolo[1,2-a][1,5]diazocin-5-yl)carbamoyl)-1H-indol-5-yl)methyl)phosphonic acid (3) utilizing the appropriate starting materials and modifications.

TABLE 29

| Compound | Name | Structure | LCMS | NMR |
|---|---|---|---|---|
| 423 | propyl ((fluoro(7- (((5S,8S,10aR)-3- (imidazo[1,5-a] pyridine-1- carbonyl)-8- (morpholine- 4-carbonyl)-6- oxodecahydro- pyrrolo [1,2- a][1,5]diazocin-5- yl)carbamoyl) naphthalen- 2-yl)methyl) (phenoxy) phosphoryl)- L-alaninate | 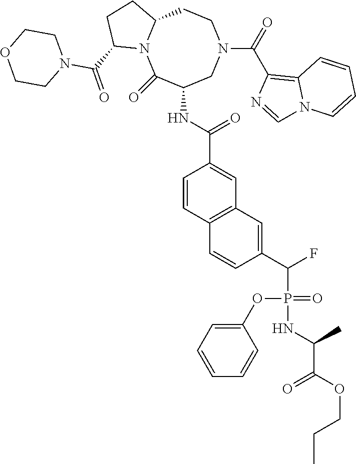 [SMILES: CCCOC(=O)[C@H](C)NP(=O)(Oc1ccccc1)C(F)c1ccc2ccc(C(=O)N[C@H]3CN(C(=O)c4ncn5ccccc45)CC[C@H]4CC[C@@H](C(=O)N5CCOCC5)N4C3=O)cc2c1] | 896.4 [M + H]⁺ | ¹H NMR (400 MHZ, CDCl₃) δ 8.48-7.41 (m, 10H), 7.39-7.18 (m, 4H), 7.13-6.98 (m, 2H), 6.82-6.68 (m, 1H), 6.15-5.87 (m, 1H), 5.72-5.12 (m, 2H), 4.98-4.75 (m, 1H), 4.68-3.90 (m, 4H), 3.88-3.46 (m, 10H), 2.71-1.77 (m, 8H), 1.69-1.38 (m, 2H), 1.36-1.03 (m, 3H), 0.97-0.71 (m, 3H) |
| 424 | propyl ((fluoro(7- (((5S,8S,10aR)-3- (6-methoxybenzo[d] isoxazole-3- carbonyl)- 8-(morpholine- 4-carbonyl)-6- oxodeca- hydropyrrolo [1,2-a][1,5] diazocin-5- yl)carbamoyl) naphthalen- 2-yl)methyl) (phenoxy) phosphoryl)-L- alaninate | 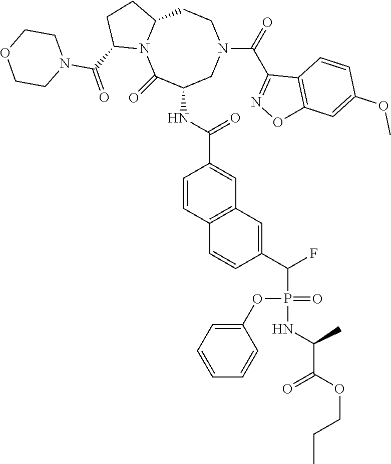 [SMILES: CCCOC(=O)[C@H](C)NP(=O)(Oc1ccccc1)C(F)c1ccc2ccc(C(=O)N[C@H]3CN(C(=O)c4noc5cc(OC)ccc45)CC[C@H]4CC[C@@H](C(=O)N5CCOCC5)N4C3=O)cc2c1] | 927.4 [M + H]⁺ | ¹H NMR (400 MHZ, CDCl₃) δ 8.48-8.23 (m, 1H), 8.14-7.78 (m, 5H), 7.77-7.46 (m, 2H), 7.39-7.28 (m, 1H), 7.26-7.15 (m, 2H), 7.13-7.00 (m, 3H), 7.00-6.92 (m, 1H), 6.14-5.90 (m, 1H), 5.40-5.23 (m, 1H), 4.89-4.75 (m, 1H), 4.66-4.35 (m, 2H), 4.24-3.38 (m, 17H), 2.38-1.86 (m, 6H), 1.59-1.56 (m, 1H), 1.50-1.41 (m, 1H), 1.34-1.19 (m, 3H), 0.96-0.73 (m, 3H) |
| 425 | propyl ((fluoro(7- (((5S,8S,10aR)-8- (morpholine- 4-carbonyl)- 6-oxo-3-(4,5,6,7- tetrahydro- benzo[d] isoxazole-3- carbonyl) decahydropyrrolo [1,2-a][1,5] diazocin- 5-yl)carbamoyl) naphthalen-2- yl)methyl) (phenoxy) phosphoryl)- L-alaninate | 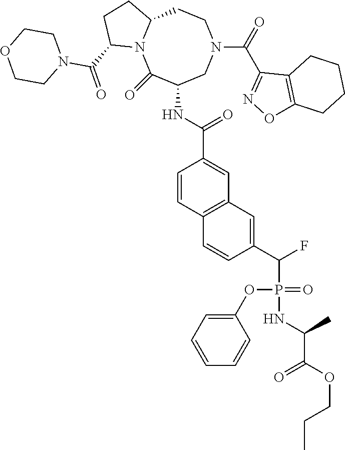 [SMILES: CCCOC(=O)[C@H](C)NP(=O)(Oc1ccccc1)C(F)c1ccc2ccc(C(=O)N[C@H]3CN(C(=O)c4noc5c4CCCC5)CC[C@H]4CC[C@@H](C(=O)N5CCOCC5)N4C3=O)cc2c1] | 901.3 [M + H]⁺ | ¹H NMR (400 MHZ, DMSO-d₆) δ 8.93- 8.71 (m, 1H), 8.59-8.49 (m, 1H), 8.18-8.10 (m, 1H), 8.10-7.95 (m, 3H), 7.80-7.72 (m, 1H), 7.39-7.30 (m, 2H), 7.23-7.11 (m, 3H), 6.40-6.10 (m, 2H), 5.17-4.96 (m, 1H), 4.87-4.77 (m, 1H), 4.40-4.28 (m, 1H), 4.00-3.89 (m, 3H), 3.88-3.58 (m, 5H), 3.57-3.38 (m, 7H), 2.78-2.66 (m, 2H), 2.65-2.52 (m, 2H), 2.29 (dd, J = 19.7, 15.0 Hz, 1H), 2.19-2.04 (m, 1H), 2.02-1.83 (m, 2H), 1.82-1.66 (m, 5H), 1.65-1.54 (m, 1H), 1.54-1.34 (m, 2H), 1.20-1.09 (m, 2H), 0.99-0.90 (m, 1H), 0.86-0.73 (m, 3H). |
| 426 | propyl ((fluoro(7- (((5S,8S,10aR)-8- (morpholine- 4-carbonyl)- 6-oxo-3- (pyrazolo[1,5-a] pyrimidine- 2-carbonyl) decahydro- pyrrolo [1,2-a][1,5] diazocin-5- yl)carbamoyl) naphthalen-2- yl)methyl) (phenoxy) phosphoryl)-L- alaninate | 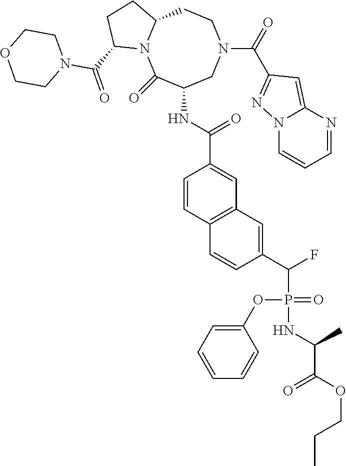 [SMILES: CCCOC(=O)[C@H](C)NP(=O)(Oc1ccccc1)C(F)c1ccc2ccc(C(=O)N[C@H]3CN(C(=O)c4cc5ncccn5n4)CC[C@H]4CC[C@@H](C(=O)N5CCOCC5)N4C3=O)cc2c1] | 897.4 [M + H]⁺ | ¹H NMR (400 MHZ, DMSO-d₆) δ 9.24- 8.86 (m, 1H), 8.70- 8.54 (m, 2H), 8.45 (s, 1H), 8.20-7.87 (m, 4H), 7.81-7.67 (m, 1H), 7.43-7.27 (m, 2H), 7.24-7.06 (m, 4H), 6.95 (s, 1H), 6.41-6.08 (m, 2H), 5.40- 5.31 (m, 1H), 4.87- 4.82 (m, 1H), 4.54- 4.21 (m, 2H), 3.97- 3.42 (m, 14H), 2.35- 1.98 (m, 3H), 1.97- 1.62 (m, 3H), 1.54- 1.30 (m, 2H), 1.22- 1.06 (m, 2H), 1.01- 0.91 (m, 1H), 0.88- 0.66 (m, 3H) |
| 427 | propyl ((fluoro(7- (((5S,8S,10aR)-8- (morpholine- 4-carbonyl)- 6-oxo-3- (propylsulfonyl) decahydro- pyrrolo [1,2-a][1,5] diazocin-5- yl)carbamoyl) naphthalen- 2-yl)methyl) (phenoxy) phosphoryl)-L- alaninate | 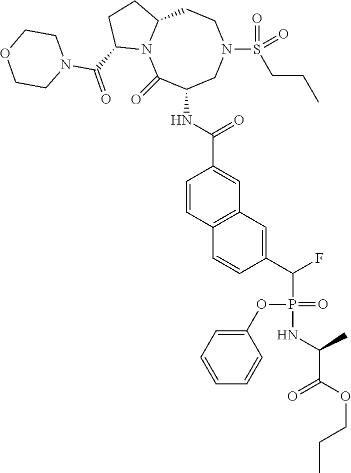 [SMILES: CCCOC(=O)[C@H](C)NP(=O)(Oc1ccccc1)C(F)c1ccc2ccc(C(=O)N[C@H]3CN(S(=O)(=O)CCC)CC[C@H]4CC[C@@H](C(=O)N5CCOCC5)N4C3=O)cc2c1] | 858.2 [M + H]⁺ | ¹H NMR (400 MHZ, DMSO-d₆) δ 8.69- 8.50 (m, 2H), 8.19- 8.10 (m, 1H), 8.09- 7.95 (m, 3H), 7.81- 7.71 (m, 1H), 7.40- 7.29 (m, 2H), 7.24- 7.10 (m, 3H), 6.38- 6.12 (m, 2H), 5.03- 4.90 (m, 1H), 4.90- 4.78 (m, 1H), 4.53- 4.39 (m, 1H), 3.97- 3.45 (m, 13H), 3.42- 3.36 (m, 1H), 3.28- 3.08 (m, 3H), 2.38- 2.26 (m, 1H), 2.19- 2.03 (m, 1H), 1.93- 1.63 (m, 6H), 1.54- 1.39 (m, 2H), 1.19- 0.96 (m, 6H), 0.86- 0.73 (m, 3H) |
| 428 | propyl ((fluoro(7- (((5S,8S,10aR)-3- (5-methoxybenzo [d]isoxazole-3- carbonyl)- 8-(morpholine- 4-carbonyl)-6- oxodecahydro- pyrrolo [1,2-a][1,5] diazocin-5- yl)carbamoyl) naphthalen-2- yl)methyl) (phenoxy) phosphoryl)-L- alaninate | 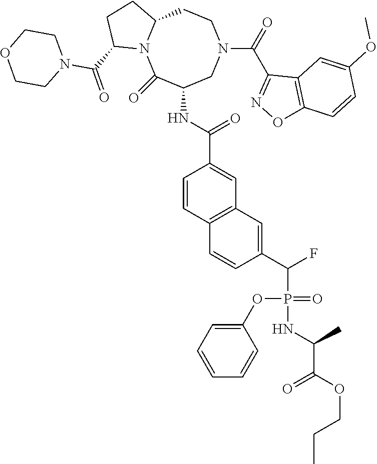 [SMILES: CCCOC(=O)[C@H](C)NP(=O)(Oc1ccccc1)C(F)c1ccc2ccc(C(=O)N[C@H]3CN(C(=O)c4noc5ccc(OC)cc45)CC[C@H]4CC[C@@H](C(=O)N5CCOCC5)N4C3=O)cc2c1] | 927.3 [M + H]⁺ | ¹H NMR (400 MHZ, DMSO-d₆) δ 8.98- 8.72 (m, 1H), 8.62- 8.45 (m, 1H), 8.19- 8.09 (m, 1H), 8.08- 7.92 (m, 3H), 7.81- 7.70 (m, 2H), 7.59- 7.46 (m, 1H), 7.39- 7.28 (m, 3H), 7.24- 7.10 (m, 3H), 6.40- 6.11 (m, 2H), 5.34- 5.15 (m, 1H), 4.86- 4.78 (m, 1H), 4.56- 4.42 (m, 1H), 4.36- 4.00 (m, 2H), 3.98- 3.88 (m, 3H), 3.87- 3.67 (m, 5H), 3.65- 3.41 (m, 7H), 3.33- 3.25 (m, 1H), 2.33- 1.58 (m, 6H), 1.53- 1.35 (m, 2H), 1.20- 0.91 (m, 3H), 0.85- 0.72 (m, 3H) |
| 429 | propyl ((fluoro(7- (((5S,8S,10aR)-8- (morpholine- 4-carbonyl)- 6-oxo-3- (5,6,7,8- tetrahydro- imidazo [1,5-a] pyridine-1- carbonyl) decahydropyrrolo [1,2-a][1,5] diazocin- 5-yl)carbamoyl) naphthalen-2- yl)methyl) (phenoxy) phosphoryl)-L- alaninate | 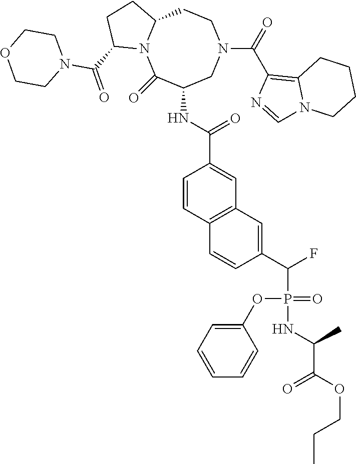 [SMILES: CCCOC(=O)[C@H](C)NP(=O)(Oc1ccccc1)C(F)c1ccc2ccc(C(=O)N[C@H]3CN(C(=O)c4ncn5c4CCCC5)CC[C@H]4CC[C@@H](C(=O)N5CCOCC5)N4C3=O)cc2c1] | 900.5 [M + H]⁺ | ¹H NMR (400 MHZ, DMSO-d₆) δ 8.93- 8.62 (m, 1H), 8.60- 8.46 (m, 1H), 8.27- 7.88 (m, 4H), 7.80- 7.67 (m, 1H), 7.63- 7.42 (m, 1H), 7.40- 7.30 (m, 2H), 7.26- 7.06 (m, 3H), 6.56- 6.09 (m, 2H), 5.35- 4.72 (m, 3H), 4.53- 4.23 (m, 1H), 4.19- 3.78 (m, 5H), 3.76- 3.66 (m, 1H), 3.65- 3.59 (m, 1H), 3.57- 3.40 (m, 6H), 3.20- 3.05 (m, 1H), 3.05- 2.84 (m, 2H), 2.36- 2.22 (m, 1H), 2.08- 1.98 (m, 1H), 1.86- 1.63 (m, 6H), 1.56- 1.36 (m, 2H), 1.32- 1.20 (m, 4H), 1.19- 1.10 (m, 2H), 1.01- 0.91 (m, 1H), 0.85- 0.73 (m, 3H) |
| 430 | propyl (((7- (((5S,8S,10aR)- 3-(5- chlorobenzo[d] isoxazole- 3-carbonyl)-8- (morpholine- 4-carbonyl)-6- oxodecahydro- pyrrolo [1,2-a][1,5] diazocin-5- yl)carbamoyl) naphthalen-2- yl)fluoromethyl) (phenoxy) phosphoryl)- L-alaninate | 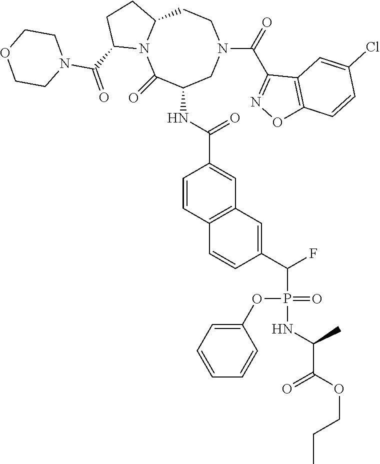 [SMILES: CCCOC(=O)[C@H](C)NP(=O)(Oc1ccccc1)C(F)c1ccc2ccc(C(=O)N[C@H]3CN(C(=O)c4noc5ccc(Cl)cc45)CC[C@H]4CC[C@@H](C(=O)N5CCOCC5)N4C3=O)cc2c1] | 931.2 [M + H]⁺ | ¹H NMR (400 MHZ, DMSO-d₆) δ 9.03- 8.76 (m, 1H), 8.67- 8.44 (m, 1H), 8.34- 8.23 (m, 1H), 8.20- 7.93 (m, 4H), 7.93- 7.86 (m, 1H), 7.81- 7.70 (m, 2H), 7.41- 7.28 (m, 2H), 7.24- 7.10 (m, 3H), 6.40- 6.09 (m, 2H), 5.45- 5.23 (m, 1H), 4.88- 4.72 (m, 1H), 4.60- 4.37 (m, 1H), 4.33- 4.05 (m, 2H), 4.04- 3.69 (m, 5H), 3.64- 3.38 (m, 8H), 2.44- 1.54 (m, 6H), 1.52- 1.34 (m, 2H), 1.21- 1.07 (m, 2H), 1.01- 0.91 (m, 1H), 0.88- 0.71 (m, 3H) |

The following compounds in Table 30 were prepared according to the representative procedure described above for the synthesis of ((7-(((5S,8S,10aR)-2-acetyl-8-((rel-trans)-3-cyano-4-phenylpyrrolidine-1-carbonyl)-6-oxodecahydropyrrolo[1,2-a][1,4]diazocin-5-yl)carbamoyl)naphthalen-2-yl)difluoromethyl)phosphonic acid (4) utilizing the appropriate starting materials and modifications.

TABLE 30

| Com- | | | | |
|---|---|---|---|---|
| pound | Name | Structure | LCMS | NMR |
| 431 | S,S′- ((((difluoro(7- (((5S,8S,10aR)- 3-(5- methyl-1H- indazole-3- carbonyl)-6- oxo-8-(6-(2- oxo-1,2- dihydropyridin- 4-y1)-4- azaspiro[2.4] heptane-4- carbonyl)dec- ahydropyrrolo [1,2-a][1,5] diazocin-5-yl) carbamoyl) naphthalen- 2-yl)methyl) phosphoryl) bis(oxy)) bis(ethane- 2,1-diyl)) | 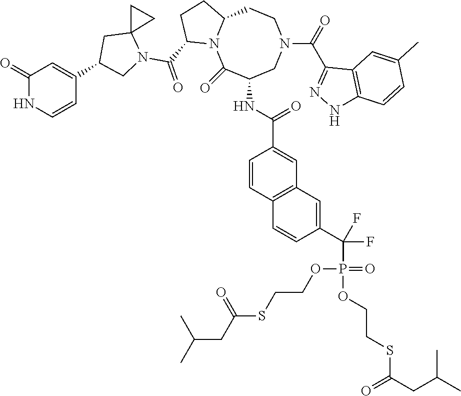 [SMILES: Cc1ccc2[nH]nc(C(=O)N3CC[C@H]4CC[C@@H](C(=O)N5C[C@H](c6cc[nH]c(=O)c6)CC56CC6)N4C(=O)[C@@H](NC(=O)c4ccc5ccc(C(F)(F)P(=O)(OCCSC(=O)CC(C)C)OCCSC(=O)CC(C)C)cc5c4)C3)c2c1] | 1130.5 [M + H]⁺ | ¹H NMR (400 MHZ, DMSO-d₆) δ 8.63 8.35 (m, 1H), 8.31- 8.11 (m, 1H), 8.10- 7.87 (m, 3H), 7.86- 7.51 (m, 2H), 7.40- 7.27 (m, 2H), 7.24- 7.09 (m, 2H), 6.84- 6.49 (m, 1H), 5.72- 5.42 (m, 1H), 4.73- 3.70 (m, 8H), 3.61- 3.25 (m, 2H), 3.25- 3.00 (m, 4H), 2.58- 2.49 (m, 2H), 2.46- 2.39 (m, 6H), 2.27- 2.01 (m, 11H), 1.94-1.82 (m, 2H), 1.67- 1.59 (m, 1H), 1.04- 0.82 (m, 12H), 0.48-0.42 (m, 2H) |
| | bis(3- | | | |
| | methylbutane | | | |
| | thioate) | | | |
| 432 | S,S′- ((((difluoro(7 (((5S,8S,10aR)- 3-(5-methyl-1H-indazole- 3-carbonyl)- 6-oxo-8-(6- (2-oxo-1,2- dihydro- pyridin-4-yl)- 4-azaspiro [2.4] heptane-4- carbonyl)dec- ahydropyrrolo [1,2a][1,5] diazocin-5- yl)carbamoyl) naphthalen-2- yl)methyl) phosphoryl) bis(oxy))bis (ethane-2,1- diyl))bis(2,2- dimethylpro- panethioate) | 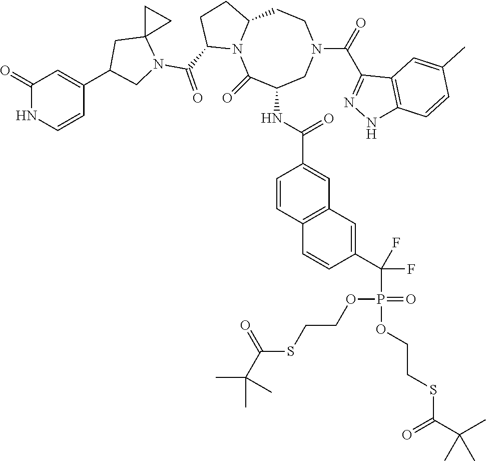 [SMILES: Cc1ccc2[nH]nc(C(=O)N3CC[C@H]4CC[C@@H](C(=O)N5CC(c6cc[nH]c(=O)c6)CC56CC6)N4C(=O)[C@@H](NC(=O)c4ccc5ccc(C(F)(F)P(=O)(OCCSC(=O)C(C)(C)C)OCCSC(=O)C(C)(C)C)cc5c4)C3)c2c1] | 1130.5 [M + H]⁺ | ¹H NMR (400 MHZ, CDCl₃) δ 8.89-7.35 (m, 8H), 7.22-6.97 (m, 2H), 6.78-5.47 (m, 2H), 4.69-2.98 (m, 16H), 2.52-2.35 (m, 4H), 2.32-1.41 (m, 11H), 1.23-1.16 (m, 18H), 0.95-0.08 (m, 2H) |
| 433 | S,S′-(((((7- (((5S,8S,10aR)- 8-((3S,4R)-3- cyano-4- phenyl- pyrrolidine-1- carbonyl)-3- (5- methylbenzo [d]isoxazole- 3-carbonyl)- 6-oxodeca- hydropyrrolo [1,2-a][1,5] diazocin-5- yl)carbamoyl) naphthalen- 2-yl) difluoromethyl) phosphoryl) bis(oxy)) bis(ethane- 2,1-diyl)) bis(3-methylbutane thioate) or | 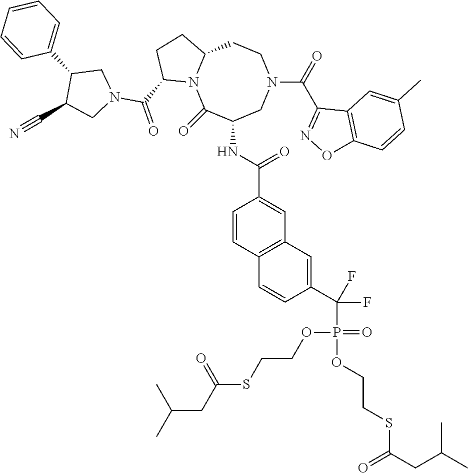 [SMILES: Cc1ccc2onc(C(=O)N3CC[C@H]4CC[C@@H](C(=O)N5C[C@@H](C#N)[C@H](c6ccccc6)C5)N4C(=O)[C@@H](NC(=O)c4ccc5ccc(C(F)(F)P(=O)(OCCSC(=O)CC(C)C)OCCSC(=O)CC(C)C)cc5c4)C3)c2c1] or | 1113.1 [M + H]⁺ | ¹H NMR (400 MHZ, DMSO-d₆) 8 9.02- 8.75 (m, 1H), 874- 8.59 (m, 1H), 8.35- 8.22 (m, 1H), 8.19- 7.96 (m, 4H), 7.74- 7.65 (m, 2H), 7.58- 7.44 (m, 1H), 7.42- 7.28 (m, 5H), 5.37- 5.22 (m, 1H), 4.61- 4.45 (m, 2H), 4.26- 3.94 (m, 9H), 3.86- 3.46 (m, 5H), 3.16- 3.10 (m, 4H), 2.48- 2.34 (m, 7H), 2.19- 1.84 (m, 7H), 1.76- 1.44 (m, 1H), 0.87- 0.83 (m, 12H) |
| | S,S'-(((((7- (((5S,8S,10aR)- 8-((3R,4S)-3- cyano-4- phenyl- pyrrolidine-1- carbonyl)-3-(5- methylbenzo [d]isoxazole- 3-carbonyl)-6- oxodeca- hydropyrrolo [1,2-a][1,5] diazocin-5- yl)carbamoyl) naphthalen- 2-yl) difluoromethyl) phosphoryl) bis(oxy)) bis(ethane- 2,1-diyl)) bis(3- methylbutane thioate) | 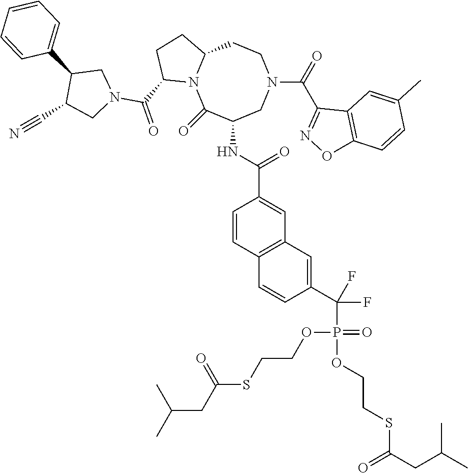 [SMILES: Cc1ccc2onc(C(=O)N3CC[C@H]4CC[C@@H](C(=O)N5C[C@H](c6ccccc6)[C@@H](C#N)C5)N4C(=O)[C@@H](NC(=O)c4ccc5ccc(C(F)(F)P(=O)(OCCSC(=O)CC(C)C)OCCSC(=O)CC(C)C)cc5c4)C3)c2c1] SFC peak 2 was used for biological testing | | |
| 434 | S,S′- ((((difluoro(7 (((5S,8S,10aR)- 3-(5- methylbenzo [d]isoxazole- 3-carbonyl)- 6-oxo-8-((S)- 6-(2-oxo-1,2- dihydropyridin- 4-yl)-4- azaspiro[2.4] heptane-4- carbonyl) decahydro-pyrrolo [1,2-a][1,5] diazocin-5- yl)carbamoyl) naphthalen-2- yl)methyl) phosphoryl)bis (oxy))bis (ethane-2,1-diyl)) bis(2,2- dimethyl- propanethioate) | 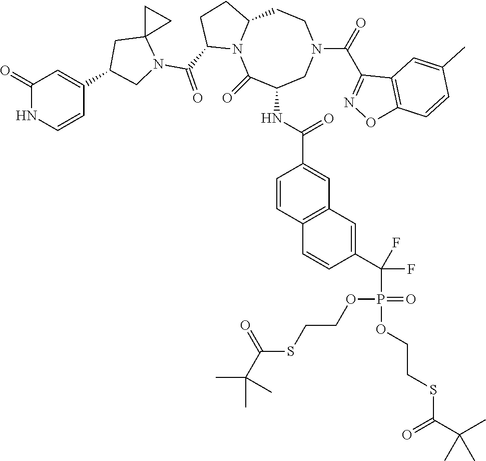 [SMILES: Cc1ccc2onc(C(=O)N3CC[C@H]4CC[C@@H](C(=O)N5C[C@H](c6cc[nH]c(=O)c6)CC56CC6)N4C(=O)[C@@H](NC(=O)c4ccc5ccc(C(F)(F)P(=O)(OCCSC(=O)C(C)(C)C)OCCSC(=O)C(C)(C)C)cc5c4)C3)c2c1] | 1131.6 [M + H]⁺ | ¹H NMR (400 MHZ, DMSO-d₆) δ 11.40(s, 1H), 9.02-8.76 (m, 1H), 8.68-8.58 (m, 1H), 8.36-7.91 (m, 5H), 7.75-7.66 (m, 2H), 7.57-7.48 (m, 1H), 7.30-7.24 (m, 1H), 6.24-6.19 (m, 1H), 6.18-6.11 (m, 1H), 5.32-5.19 (m, 1H), 4.59-4.49 (m, 1H), 4.46-4.36 (m, 1H), 4.31-3.79 (m, 10H), 3.59-3.48 (m, 1H), 3.14-3.07 (m, 4H), 2.47 (s, 3H), 2.34-1.22 (m, 12H), 1.15-1.11 (m, 18H) |
| 435 | S,S′- ((((difluoro(7 (((5S,8S,10aR)- 3-(5- methylbenzo [d]isoxazole-3-carbonyl)-6- oxo-8-((R)-6-(2-oxo-1,2- dihydro-pyridin- 4-yl)-4- azaspiro[2.4] heptane-4- carbonyl) decahydro-pyrrolo[1,2-a] [1,5]diazocin- 5-yl) carbamoyl) naphthalen-2- yl)methyl) phosphoryl) bis(oxy))bis (ethane-2,1- diyl))bis(2,2- dimethyl- propanethioate) | 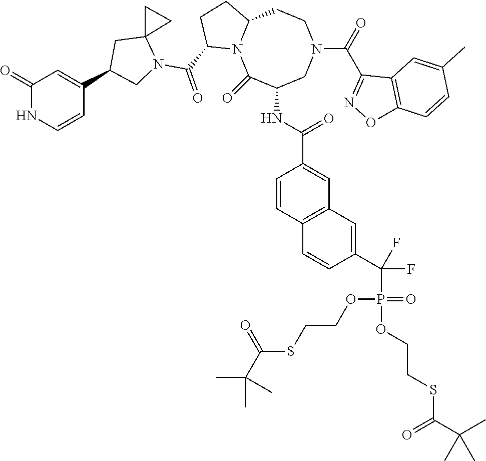 [SMILES: Cc1ccc2onc(C(=O)N3CC[C@H]4CC[C@@H](C(=O)N5C[C@@H](c6cc[nH]c(=O)c6)CC56CC6)N4C(=O)[C@@H](NC(=O)c4ccc5ccc(C(F)(F)P(=O)(OCCSC(=O)C(C)(C)C)OCCSC(=O)C(C)(C)C)cc5c4)C3)c2c1] | 1131.7 [M + H]⁺ | ¹H NMR (400 MHZ, DMSO-d₆) δ 11.38 (s,1H), 9.04-8.78 (m, 1H), 8.76-8.60 (m, 1H), 8.32-7.95 (m, 5H), 7.78-7.68 (m, 2H), 7.56-7.49 (m, 1H), 7.29-7.21 (m, 1H), 6.25-6.09 (m, 2H), 5.30-5.20 (m, 1H), 4.53-4.37 (m, 2H), 4.25-4.09 (m, 6H), 4.05-3.85 (m, 4H), 3.67-3.53 (m, 1H), 3.12-3.06 (m, 4H), 2.47 (s, 3H), 2.10-1.40 (m, 12H), 1.14- 1.11 (m, 18H), 0.50-0.14 (m, 2H) |
| 436 | S,S′- ((((difluoro(7- (((5S,8S,10aR)- 8-((R)-6- (1-methyl-2- oxo-1,2- dihydropyridin- 4-yl)-4- azaspiro[2.4] heptane-4- carbonyl)-3-(5- methylbenzo [d]isoxazole- 3-carbonyl)-6- oxodeca- hydropyrrolo [1,2-a][1,5] diazocin-5- yl)carbamoyl) naphthalen-2- yl)methyl) phosphoryl)bis (oxy))bis (ethane-2,1-diyl))bis(2,2- dimethyl- propanethioate) | 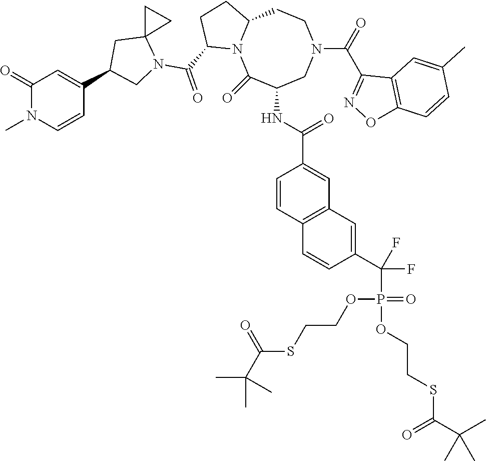 [SMILES: Cc1ccc2onc(C(=O)N3CC[C@H]4CC[C@@H](C(=O)N5C[C@@H](c6ccn(C)c(=O)c6)CC56CC6)N4C(=O)[C@@H](NC(=O)c4ccc5ccc(C(F)(F)P(=O)(OCCSC(=O)C(C)(C)C)OCCSC(=O)C(C)(C)C)cc5c4)C3)c2c1] | 1145.3 [M + H]⁺ | ¹H NMR (400 MHZ, CDCl₃) δ 8.55-8.27 (m, 1H), 8.24-8.12 (m, 1H), 8.11-7.99 (m, 1H), 7.97-7.59 (m, 5H), 7.53-7.34 (m, 2H), 7.24-7.17 (m, 1H), 6.47-6.40 (m, 1H), 6.11-6.00 (m, 1H), 5.46-5.25 (m, 1H), 4.68-3.87 (m, 11H), 3.57-3.31 (m, 5H), 3.21-3.02 (m, 4H), 2.53-2.41 (m, 3H), 2.36-1.92 (m, 10H), 1.28-1.14 (m, 18H), 0.58-0.13 (m, 2H) |
| 437 | S,S′- ((((difluoro(7 (((5S,8S,10aR)- 8-((S)-6- (1-methyl-2- oxo-1,2- dihydropyridin- 4-yl)-4- azaspiro[2.4] heptane-4- carbonyl)-3-(5- methylbenzo [d]isoxazole- 3-carbonyl)-6- oxodeca- hydropyrrolo [1,2-a][1,5] diazocin-5- yl)carbamoyl) naphthalen-2- yl)methyl) phosphoryl)bis (oxy))bis (ethane-2,1-diyl))bis(2,2- dimethyl- propanethioate) | 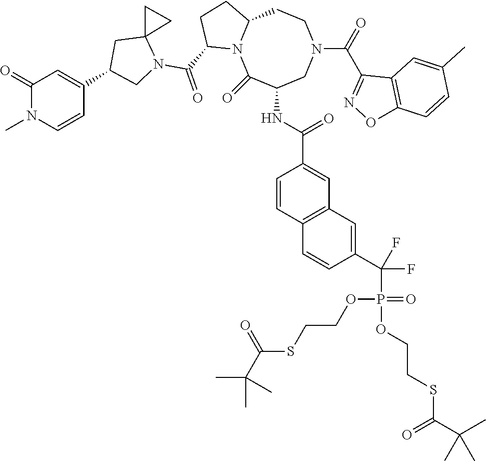 [SMILES: Cc1ccc2onc(C(=O)N3CC[C@H]4CC[C@@H](C(=O)N5C[C@H](c6ccn(C)c(=O)c6)CC56CC6)N4C(=O)[C@@H](NC(=O)c4ccc5ccc(C(F)(F)P(=O)(OCCSC(=O)C(C)(C)C)OCCSC(=O)C(C)(C)C)cc5c4)C3)c2c1] | 1145.2 [M + H]⁺ | ¹H NMR (400 MHZ, CDCl₃) δ 8.50-8.33 (m, 1H), 8.25-8.12 (m, 1H), 8.04-7.85 (m, 4H), 7.79-7.62 (m, 2H), 7.53-7.43 (m, 1H), 7.42-7.33 (m, 1H), 7.25-7.18 (m, 1H), 6.56-6.44 (m, 1H), 6.20-6.08 (m, 1H), 5.39-5.21 (m, 1H), 4.61-4.29 (m, 3H), 4.26-3.82 (m, 9H), 3.48 (s, 3H), 3.38-3.24 (m, 1H), 3.15-3.06 (m, 4H), 2.47 (s, 3H), 2.33- 2.02 (m, 7H), 1.94- 1.86 (m, 2H), 1.67- 1.48 (m, 1H), 1.21- 1.19 (m, 18H), 0.55-0.24 (m, 2H) |
| 438 | S,S′- ((((difluoro(7 (((5S,8S,10aR)- 8-((S)-6- (1-methyl-2- oxo-1,2- dihydropyridin- 4-yl)-4- azaspiro[2.4] heptane-4- carbonyl)-3-(5- methylbenzo [d]isoxazole- 3-carbonyl)-6- oxodeca- hydropyrrolo [1,2-a][1,5] diazocin-5- yl)carbamoyl) naphthalen-2- yl)methyl) phosphoryl)bis (oxy))bis (ethane-2,1- | 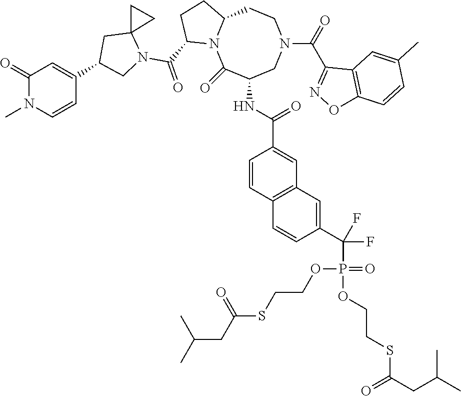 [SMILES: Cc1ccc2onc(C(=O)N3CC[C@H]4CC[C@@H](C(=O)N5C[C@H](c6ccn(C)c(=O)c6)CC56CC6)N4C(=O)[C@@H](NC(=O)c4ccc5ccc(C(F)(F)P(=O)(OCCSC(=O)CC(C)C)OCCSC(=O)CC(C)C)cc5c4)C3)c2c1] | 1145.4 [M + H]⁺ | ¹H NMR (400 MHZ, CDCl₃) δ 8.53-8.36 (m, 1H), 8.25-8.16 (m, 1H), 8.08-7.84 (m, 4H), 7.78-7.73 (m, 1H), 7.68-7.44 (m, 2H), 7.42-7.37 (m, 1H), 7.31-7.27 (m, 1H), 6.65-6.56 (m, 1H), 6.28-6.15 (m, 1H), 5.36-5.28 (m, 1H), 4.76-3.82 (m, 12H), 3.57-3.49 (m, 3H), 3.42-3.31 (m, 1H), 3.21-3.07 (m, 4H), 2.57-2.44 (m, 7H), 2.23-1.91 (m, 9H), 1.73-1.60 (m, 5H), 0.97-0.89 (m, 6H), 0.55-0.22 (m, 2H) |
| | diyl))bis(3- | | | |
| | methylbutane | | | |
| | thioate) | | | |
| 439 | S,S′- ((((difluoro(7 (((5S,8S,10aR)- 8-((S)-6- (1-methyl-2- oxo-1,2- dihydropyridin- 4-yl)-4- azaspiro[2.4] heptane-4- carbonyl)-3-(5- methylbenzo [d]isoxazole- 3-carbonyl)-6- oxodeca- hydropyrrolo [1,2-a][1,5] diazocin-5- yl)carbamoyl) naphthalen-2- yl)methyl) phosphoryl)bis (oxy))bis (ethane-2,1- | 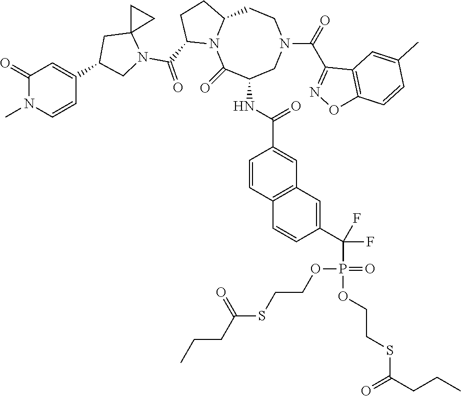 [SMILES: CCCC(=O)SCCOP(=O)(OCCSC(=O)CCC)C(F)(F)c1ccc2ccc(C(=O)N[C@H]3CN(C(=O)c4noc5ccc(C)cc45)CC[C@H]4CC[C@@H](C(=O)N5C[C@H](c6ccn(C)c(=O)c6)CC56CC6)N4C3=O)cc2c1] | 1117.3 [M + H]⁺ | ¹H NMR (400 MHZ, CDCl₃) δ 8.53-8.36 (m, 1H), 8.25-8.16 (m, 1H), 8.08-7.84 (m, 4H), 7.78-7.73 (m, 1H), 7.68-7.44 (m, 2H), 7.42-7.37 (m, 1H), 7.31-7.27 (m, 1H), 6.65-6.56 (m, 1H), 6.28-6.15 (m, 1H), 5.36-5.28 (m, 1H), 4.76-3.82 (m, 12H), 3.57-3.49 (m, 3H), 3.42-3.31 (m, 1H), 3.21-3.07 (m, 4H), 2.57-2.44 (m, 7H), 2.23-1.91 (m, 9H), 1.73-1.60 (m, 5H), 0.97-0.89 (m, 6H), 0.55-0.22 (m, 2H) |
| | diyl)) | | | |
| | dibutanethioate | | | |
| 440 | propyl (((7- (((5S,8S,10aR)- 8-((2R,6S)-2,6- dimethyl- morpholine-4- carbonyl)-3- (5-methylbenzo [d]isoxazole-3-carbonyl)-6- oxodeca- hydropyrrolo [1,2-a][1,5] diazocin-5-yl)carbamoyl) naphthalen- 2-yl) fluoromethyl) | 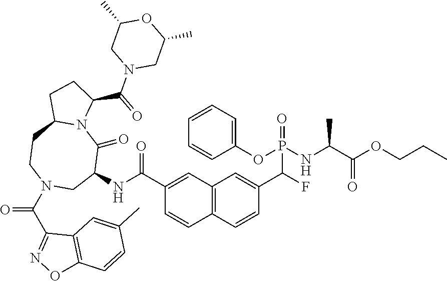 [SMILES: CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N5C[C@@H](C)O[C@@H](C)C5)N4C3=O)cc2c1] | 939.4 | ¹H NMR (400 MHZ, CDCl₃) δ 8.29 (m, 1H), 8.02-7.94 (m, 1H), 7.89-7.79 (m, 3H), 7.75-7.64 (m, 1H), 7.58-7.49 (m, 1H), 7.42-7.31 (m, 2H), 7.31-7.16 (m, 4H), 7.12-6.98 (m, 1H), 6.14-5.92 (m, 1H), 5.42-5.30 (m, 1H), 4.86 (s, 1H), 4.65-4.43 (m, 2H), 4.39- 4.24 (m, 1H), 4.19- 4.10 (m, 1H), 4.07- 3.90 (m, 3H), 3.90- |
| | (phenoxy) | | | 3.56 (m, 4H), 3.50 |
| | phosphoryl)- | | | (m, 1H), 2.85 (m, |
| | L-alaninate | | | 2H), 2.47 (s, 3H), |
| | | | | 2.37-2.13 (m, 4H), |
| | | | | 2.07-1.87 (m, 2H), |
| | | | | 1.64-1.41 (m, 2H), |
| | | | | 1.33-1.24 (m, 3H), |
| | | | | 1.23-1.15 (m, 4H), |
| | | | | 1.12-1.03 (m, 2H), |
| | | | | 0.95-0.74 (m, 3H) |
| 441 | propyl (((2- (((5S,8S,10aR)- 8-((3S,4R)-3- cyano-4- phenyl- pyrrolidine-1- carbonyl)-3- (2,2- difluoroethyl)- 6- oxodeca- hydropyrrolo [1,2-a][1,5] diazocin-5- yl)carbamoyl) benzo[b] thiophen-5-yl) fluoromethyl) (phenoxy) L-alaninate | 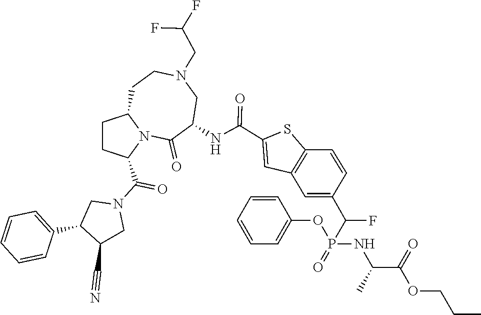 [SMILES: CCCOC(=O)[C@H](C)NP(=O)(Oc1ccccc1)C(F)c1ccc2sc(C(=O)N[C@H]3CN(CC(F)F)CC[C@H]4CC[C@@H](C(=O)N5C[C@@H](C#N)[C@H](c6ccccc6)C5)N4C3=O)cc2c1] | 907.5 | ¹H NMR (400 MHZ, CDCl₃) δ 7.99 (d, J = 10.8 Hz, 1H), 7.93-7.82 (m, 2H), 7.65-7.52 (m, 2H), 7.48-7.32 (m, 6H), 7.27-7.23 (m, 1H), 7.18-7.03 (m, 2H), 6.02 (d, J = 12.4 Hz, 2H), 5.03 (s, 1H), 4.63 (s, 1H), 4.50 (s, 1H), 4.34-3.29 (m, 1H), 4.07 (d, J = 7.2 Hz, 3H), 3.84 (s, 1H), 3.76-3.70 (m, 2H), 3.60-3.52 (m, 1H), 3.44 (d, J = 12.4 Hz, 2H), 3.25 (d, J = 8.0 Hz, 4H), 3.17-3.10 (m, 1H), 2.41-2.25 (m, 1H), |
| | | | | 2.20-2.13 (m, 1H), |
| | | | | 1.99-1.88 (m, 2H), |
| | | | | 1.64 (d, J = 7.6 |
| | | | | Hz, 1H), 1.51-1.45 |
| | | | | (m, 2H), 1.42 (d, J = |
| | | | | 6.4 Hz, 1H), |
| | | | | 1.351.14(m, 3H), |
| | | | | 0.96-0.84 (m,3H) |

### Biochemical and Cellular Assays

### STAT3 Fluorescence Polarization (FP) Assay

An FP assay was developed to determine IC₅₀ values for test substances. Recombinant STAT3 protein (STAT3(G127-I722)) at 25 nM was combined with a fluorescently labeled, phosphotyrosine peptide probe (5-FAM-GpYLPQTV-NH₂) at 2 nM in FP buffer (10 mM HEPES pH 7.4, 50 mM NaCl, 1 mM EDTA, 0.05% Tween 20.2 mM DTT). 50 μL of STAT3-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 31. For STAT3 FP assay, A=≤100 nM; B=>100-1000 nM; C=>1-10 μM; and D=>10 μM.

### STAT6 Fluorescence Polarization (FP) Assay

An FP assay was developed to determine IC₅₀ values for test substances. Recombinant STAT6 protein (STAT6(W123-T658)) at 250 nM was combined with a fluorescently labeled, phosphotyrosine peptide probe (5-FAM-ApYKPFQDLI-NH₂) at 2 nM in FP buffer (10 mM HEPES pH 7.4, 50 mM NaCl, 1 mM EDTA, 0.05% Tween 20.2 mM DTT). 50 μL of STAT6-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 31. For STAT6 FP assay, A=≤300 nM; B=>300-3000 nM; C=>3-30 μM; and D=>30 μM.

TABLE 31

| Cmp# | STAT3 | STAT6 |
|---|---|---|
| 2 | D | B |
| 3 | C | B |
| 4 | C | A |
| 5 | C | B |
| 6 | C | B |
| 7 | C | B |
| 8 | B | A |
| 9 | B | A |
| 10 | C | A |
| 11 | B | B |
| 12 | A | A |
| 13 | C | B |
| 14 | D | B |
| 15 | D | C |
| 16 | D | D |
| 17 | C | B |
| 18 | D | C |
| 19 | D | D |
| 20 | D | D |
| 21 | D | D |
| 22 | D | B |
| 23 | C | B |
| 24 | B | B |
| 25 | D | C |
| 26 | D | D |
| 27 | C | C |
| 28 | C | B |
| 29 | C | B |
| 30 | C | B |
| 31 | C | B |
| 32 | C | C |
| 33 | C | C |
| 34 | B | B |
| 35 | C | B |
| 36 | C | B |
| 37 | D | A |
| 38 | C | A |
| 39 | C | A |
| 40 | A | C |
| 41 | C | A |
| 42 | C | B |
| 43 | C | B |
| 44 | B | C |
| 45 | D | D |
| 46 | C | B |
| 47 | C | B |
| 48 | B | B |
| 49 | C | C |
| 50 | D | C |
| 51 | D | C |
| 52 | C | A |
| 53 | C | B |
| 54 | C | B |
| 55 | C | B |
| 56 | C | B |
| 57 | C | B |
| 58 | B | B |
| 59 | C | A |
| 60 | C | A |
| 61 | B | B |
| 62 | C | B |
| 63 | C | B |
| 64 | C | B |
| 65 | C | B |
| 67 | C | B |
| 68 | B | B |
| 69 | C | B |
| 70 | C | B |
| 71 | C | B |
| 72 | C | B |
| 73 | C | C |
| 75 | C | C |
| 76 | B | B |
| 77 | B | C |
| 78 | B | B |
| 79 | B | B |
| 80 | B | B |
| 81 | B | B |
| 82 | C | B |
| 83 | C | C |
| 84 | B | B |
| 85 | C | B |
| 86 | A | B |
| 87 | C | B |
| 88 | A | B |
| 89 | C | B |
| 90 | C | B |
| 91 | C | B |
| 92 | C | B |
| 93 | C | B |
| 94 | C | B |
| 95 | B | B |
| 96 | C | A |
| 97 | B | A |
| 99 | B | B |
| 100 | B | B |
| 101 | C | B |
| 102 | C | B |
| 103 | C | C |
| 104 | C | B |
| 105 | B | B |
| 106 | C | B |
| 107 | C | A |
| 108 | C | B |
| 109 | C | B |
| 110 | C | C |
| 111 | D | B |
| 112 | D | D |
| 113 | D | D |
| 114 | D | D |
| 115 | D | B |
| 116 | B | B |
| 117 | D | B |
| 118 | D | B |
| 119 | D | B |
| 120 | D | C |
| 121 | D | C |
| 122 | A | B |
| 123 | C | C |
| 124 | C | A |
| 125 | C | B |
| 126 | C | B |
| 127 | B | B |
| 128 | B | B |
| 129 | C | B |
| 130 | C | B |
| 131 | C | B |
| 132 | C | B |
| 133 | C | B |
| 134 | C | B |
| 135 | C | B |
| 136 | C | A |
| 137 | C | B |
| 138 | C | A |
| 139 | C | A |
| 140 | B | B |
| 141 | C | B |
| 142 | C | A |
| 143 | C | A |
| 144 | C | A |
| 145 | B | A |
| 146 | B | B |
| 147 | B | B |
| 148 | C | B |
| 149 | C | A |
| 150 | B | A |
| 151 | C | B |
| 152 | C | A |
| 153 | C | B |
| 154 | C | B |
| 155 | C | B |
| 156 | C | B |
| 157 | C | B |
| 158 | B | B |
| 159 | C | B |
| 160 | C | B |
| 161 | C | B |
| 162 | C | B |
| 163 | C | B |
| 164 | C | B |
| 165 | C | B |
| 166 | C | B |
| 167 | C | B |
| 168 | C | B |
| 169 | C | B |
| 170 | C | B |
| 171 | C | B |
| 172 | C | B |
| 173 | B | B |
| 174 | B | A |
| 175 | B | B |
| 176 | B | B |
| 177 | A | B |
| 178 | B | B |
| 179 | B | B |
| 180 | B | B |
| 181 | C | B |
| 182 | B | B |
| 183 | C | B |
| 184 | B | B |
| 185 | C | B |
| 186 | C | A |
| 187 | C | B |
| 188 | C | B |
| 189 | C | B |
| 190 | C | B |
| 191 | B | A |
| 192 | C | C |
| 193 | B | B |
| 194 | C | B |
| 195 | C | B |
| 196 | B | B |
| 197 | B | B |
| 198 | B | B |
| 199 | C | B |
| 200 | C | B |
| 201 | C | B |
| 202 | C | B |
| 203 | C | A |
| 204 | B | B |
| 205 | C | B |
| 206 | C | B |
| 207 | C | B |
| 208 | A | B |
| 209 | C | B |
| 210 | A | C |
| 211 | C | A |
| 212 | C | C |
| 213 | C | A |
| 214 | A | C |
| 215 | B | A |
| 216 | C | B |
| 217 | D | B |
| 218 | C | B |
| 219 | C | B |
| 220 | D | B |
| 221 | C | B |
| 222 | C | B |
| 223 | C | B |
| 224 | C | C |
| 225 | C | C |
| 226 | A | B |
| 227 | C | A |
| 228 | C | A |
| 229 | C | A |
| 230 | D | C |
| 231 | C | A |
| 232 | B | A |
| 233 | C | A |
| 234 | D | A |
| 235 | A | D |
| 236 | B | A |
| 237 | B | A |
| 238 | C | B |
| 240 | C | B |
| 241 | C | B |
| 242 | B | A |
| 243 | C | B |
| 244 | C | B |
| 245 | B | B |
| 246 | C | B |
| 247 | B | A |
| 248 | C | A |
| 249 | B | A |
| 250 | B | A |
| 251 | B | B |
| 252 | C | B |
| 253 | C | A |
| 254 | C | B |
| 255 | B | B |
| 256 | B | C |
| 257 | B | C |
| 258 | C | B |
| 259 | B | B |
| 260 | B | B |
| 261 | B | B |
| 262 | C | B |
| 263 | A | C |
| 264 | C | B |
| 265 | C | B |
| 266 | B | A |
| 267 | C | B |
| 268 | C | B |
| 269 | A | B |
| 270 | C | A |
| 271 | C | A |
| 272 | A | C |
| 273 | A | C |
| 274 | A | C |
| 275 | A | C |
| 276 | C | B |
| 277 | A | C |
| 278 | B | C |
| 279 | B | A |
| 354 | D | A |
| 355 | C | B |
| 356 | D | B |
| 357 | D | B |
| 358 | D | B |
| 359 | D | B |
| 360 | D | B |
| 361 | D | C |
| 362 | D | A |
| 363 | D | A |
| 364 | D | B |
| 365 | D | A |
| 366 | D | B |
| 367 | C | A |
| 368 | D | A |
| 369 | C | A |
| 370 | D | A |
| 371 | D | A |
| 372 | D | A |
| 373 | D | A |
| 374 | D | A |
| 375 | C | A |
| 376 | D | B |
| 377 | D | A |
| 378 | D | B |
| 379 | D | B |
| 380 | D | A |
| 381 | C | A |
| 382 | C | A |
| 383 | C | A |
| 384 | D | B |
| 385 | C | A |
| 386 | D | A |
| 387 | D | A |
| 388 | D | B |
| 389 | D | B |
| 390 | C | A |
| 391 | D | A |
| 392 | C | C |
| 393 | D | D |
| 394 | D | C |
| 395 | C | A |
| 396 | D | C |
| 397 | D | A |
| 398 | A | C |
| 399 | B | D |
| 400 | B | C |
| 401 | A | C |
| 402 | A | C |
| 403 | A | C |
| 404 | A | C |
| 405 | A | C |

### MSD-pSTAT-PBMC Assay

### Materials:

Cryopreserved Peripheral Blood Mononuclear Cells (PBMC) are from AllCells. Recombinant Human IL-4 and IL-6 are from Peprotech. Mouse monoclonal anti-STAT3 antibody, rabbit monoclonal anti-pY705-STAT3 antibody, rabbit monoclonal Anti-pY641-STAT6 antibody, and lysis buffer are from Cell Signaling Technology (CST). Mouse monoclonal anti-STAT6 antibody is from BioLegend. Assay plates, blocker, and anti-rabbit secondary antibody are from Meso Scale Discovery (MSD).

### Assay Method:

Cryopreserved PBMCs were thawed out and allowed to recover overnight in IMDM+10% heat-inactivated FBS prior to plating 50,000 (STAT3) or 25,000 (STAT6) cells per well in 96-well U-bottom tissue culture plates. Cells were treated with compound for 3 hrs, then stimulated with 10 ng/mL IL-6 (STAT3) or 1 ng/mL IL-4 (STAT6) for 10 min. Cells were then spun down and washed with ice-cold PBS prior to lysing the cell pellet with 1× lysis buffer (CST) with 1×HALT protease and phosphatase inhibitor cocktail (Thermo). Lysates were transferred to and incubated overnight at 4° C. with shaking in QuickPlex 96-well high bind assay plates (MSD) pre-coated overnight with 30 μL per well of 0.6 μg/mL mouse monoclonal anti-STAT3 antibody (STAT3) or 2 μg/mL mouse monoclonal anti-STAT6 antibody (STAT6) in 1×PBS, and pre-blocked for 1 hour with 3% Blocker-A (MSD). Captured protein in the assay plates were then washed and probed with 25 μL per well of 0.25 μg/ml rabbit monoclonal anti-pY705-STAT3 antibody (STAT3) or 0.18 pg/ml rabbit monoclonal Anti-pY641-STAT6 antibody (STAT6) in 1% Blocker-A for 1 hour at room temperature with shaking, then washed and probed with 25 ul per well of 1 ug/ml Sulfo-TAG Labeled Goat Anti-Rabbit Antibody (MSD) in 1% Blocker-A for 1 hour at room temperature with shaking. Assay plates were then washed and 150 μL of 1× Read Buffer-T (MSD) was added to each well prior to reading on an SQ120 MSD Plate Reader. Assay signal from each sample was subtracted by the signal from unstimulated control wells and normalized to DMSO control wells. IC₅₀ values were calculated using Graph Pad Prism Dose-Response Nonlinear Regression with variable slope. Results are shown in Table 32. For both pSTAT3 and pSTAT6 A=≤100 nM; B=>100-1000 nM; C=>1000-3000 nM; and D=>3 μM.

TABLE 32

| Cmp# | pSTAT3 | pSTAT6 |
|---|---|---|
| 1 | D | B |
| 239 | B | B |
| 280 | C | B |
| 281 | A | A |
| 282 | C | A |
| 283 | D | C |
| 284 | B | A |
| 285 | B | A |
| 286 | B | B |
| 287 | D | D |
| 288 | D | D |
| 289 | D | D |
| 290 | C | B |
| 291 | D | D |
| 292 | D | D |
| 293 | D | D |
| 294 | B | B |
| 295 | B | B |
| 296 | B | A |
| 300 | B | B |
| 302 | | B |
| 303 | C | B |
| 304 | D | B |
| 305 | C | D |
| 306 | D | C |
| 307 | D | D |
| 308 | B | B |
| 309 | D | C |
| 310 | B | B |
| 311 | C | B |
| 312 | D | B |
| 313 | D | C |
| 314 | C | B |
| 315 | D | B |
| 316 | D | C |
| 317 | D | D |
| 318 | B | B |
| 319 | B | A |
| 320 | B | A |
| 321 | B | A |
| 322 | D | D |
| 323 | C | A |
| 324 | D | D |
| 325 | D | D |
| 326 | D | D |
| 327 | D | D |
| 328 | D | B |
| 329 | C | A |
| 330 | C | C |
| 331 | D | A |
| 332 | D | A |
| 333 | D | |
| 334 | B | A |
| 335 | C | A |
| 336 | C | A |
| 337 | D | D |
| 338 | B | |
| 339 | C | B |
| 340 | D | D |
| 341 | D | C |
| 342 | D | B |
| 343 | C | B |
| 344 | C | B |
| 345 | C | B |
| 346 | B | A |
| 347 | B | B |
| 348 | A | A |
| 349 | B | A |
| 350 | A | B |
| 351 | A | C |
| 352 | A | A |
| 353 | A | D |
| 406 | | B |
| 407 | | C |
| 408 | | C |
| 409 | | C |
| 410 | | B |
| 411 | | D |
| 412 | | B |
| 413 | | A |
| 414 | | A |
| 415 | | B |
| 416 | | A |
| 417 | | A |
| 418 | | A |
| 419 | | A |
| 420 | | A |
| 421 | | A |
| 422 | | C |
| 423 | | D |
| 424 | | C |
| 425 | | C |
| 426 | | D |
| 427 | | D |
| 428 | | B |
| 429 | | D |
| 430 | | A |
| 431 | | A |
| 432 | | A |
| 433 | | A |
| 434 | | A |
| 435 | | A |
| 436 | | A |
| 437 | | A |
| 438 | | A |
| 439 | | A |
| 440 | | A |
| 441 | | A |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the structural formula I: 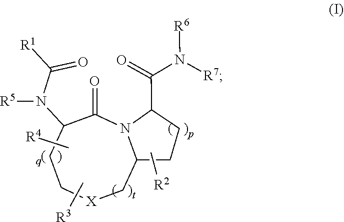
or a pharmaceutically acceptable salt thereof, wherein:
q is 0 or 1 and t is 0, 1, or 2, provided that at least one of q or t is 1;
p is 1 or 2;
X is selected from S, SO₂, —S(═O)═NH, and NR⁸;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}], wherein said aryl may be further optionally substituted with 1 or 2 groups independently selected from cyano, (C₁-C₄)alkoxy, and halo; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], —[P(O)[NH(AA)C(O)OR^{T}][NH(AA)C(O)OR^{T}], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 10-membered heteroaryl, —(C₁-C₄)[5- to 10-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 10-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, cyano, and 4- to 6-membered heterocyclyl optionally substituted with C₁-C₄alkyl;
R^{O} is selected from halo, (C₁-C₄)alkoxy, OH, phenyl, NH₂, —NH(C₁-C₁₀)alkyl, —N[(C₁-C₁₀)alkyl], (C₃₋₆cycloalkyl), 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano;
R^{Hb} is hydrogen or (C₁-C₄)alkyl;
AA is the residue of an alpha or beta natural or non-natural amino acid;
R^{T} and R^{TY} are each independently selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} and R^{U} are each independently selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R⁹, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

2. The compound of claim 1, or a pharmaceutically acceptable salt thereof, wherein
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 6-membered heteroaryl, —(C₁-C₄)[5- to 6-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 6-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano; and
R^{O} is selected from halo, (C₁-C₄)alkoxy, OH, phenyl, NH₂, —NH(C₁-C₁₀)alkyl, —N[(C₁-C₁₀)alkyl], 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano.

3. The compound of claim 1 or 2, wherein the compound is of the structural formula II: 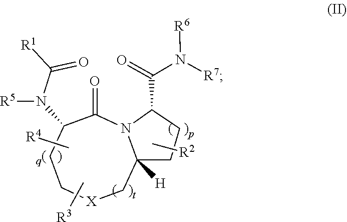
or a pharmaceutically acceptable salt thereof.

4. The compound of any one of claims 1 to 3, or a pharmaceutically acceptable salt thereof, wherein p is 1.

5. The compound of any one of claims 1 to 4, or a pharmaceutically acceptable salt thereof, wherein R² is hydrogen.

6. The compound of any one of claims 1 to 5, or a pharmaceutically acceptable salt thereof, wherein R³ is hydrogen.

7. The compound of any one of claims 1 to 6, or a pharmaceutically acceptable salt thereof, wherein R⁴ is hydrogen.

8. The compound of any one of claims 1 to 7, or a pharmaceutically acceptable salt thereof, wherein q is 1 and t is 1.

9. The compound of any one of claims 1 to 7, or a pharmaceutically acceptable salt thereof, wherein q is 0 and t is 2.

10. The compound of any one of claims 1 to 7, wherein the compound is of the structural formula III, IV, V, VI, VII, or VIII: 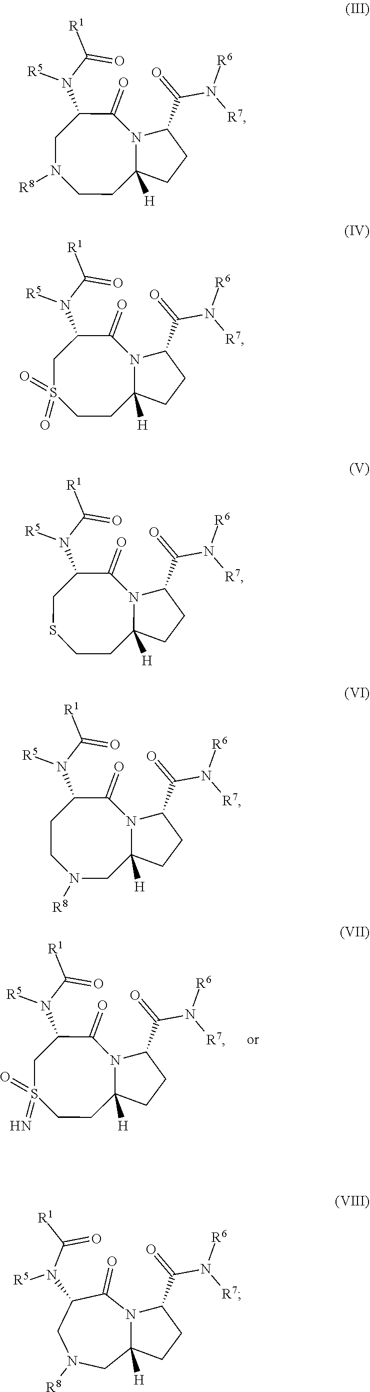
or a pharmaceutically acceptable salt thereof.

11. The compound of any one of claims 1 to 10, or a pharmaceutically acceptable salt thereof, wherein R⁵ is hydrogen.

12. The compound of any one of claims 1 to 11, or a pharmaceutically acceptable salt thereof, wherein R¹ is selected from 8- to 10-membered fused bicyclic heteroaryl and aryl, each of which are substituted with —CR^{1a}R^{2a}P(O)OR^{1b}OR^{2b}.

13. The compound of any one of claims 1 to 12, or a pharmaceutically acceptable salt thereof, wherein, R¹ is selected from benzothiophenyl, indolyl, naphthalenyl, thienopyridinyl, benzothiazoyl, quinolinyl, isoquinolinyl, 4,5,6,7-tetrahydropyrrolopyridinyl, 5,6,7,8-tetrahydroimidazopyrazinyl, each of which are substituted with —CR^{1a}R^{2a}P(O)OR^{1b}OR^{2b}.

14. The compound of any one of claims 1 to 13, or a pharmaceutically acceptable salt thereof, wherein, R¹ is selected from

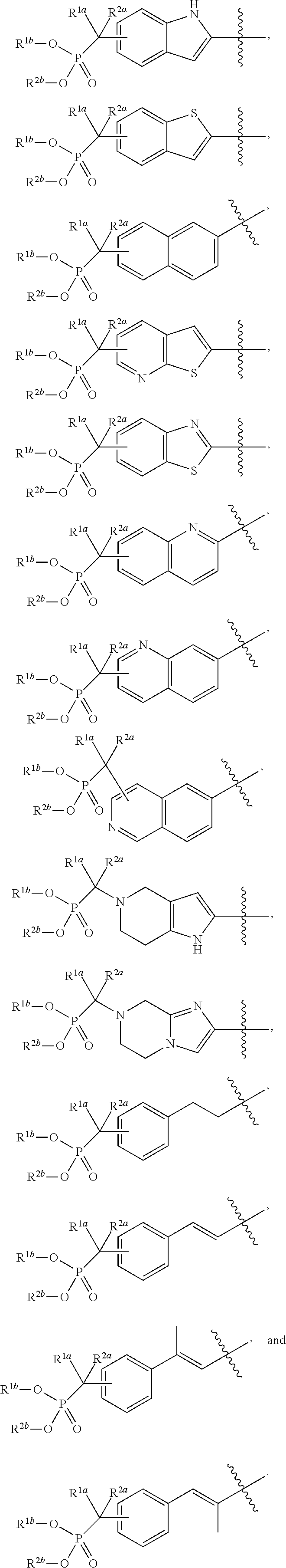

15. The compound of any one of claims 1 to 14, or a pharmaceutically acceptable salt thereof, wherein, R¹ is

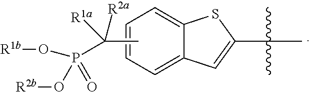

16. The compound of any one of claims 1 to 15, or a pharmaceutically acceptable salt thereof, wherein R^{1a} is hydrogen and R^{2a} is fluoro or R^{1a} is fluoro and R^{2a} is fluoro.

17. The compound of any one of claims 1 to 16, or a pharmaceutically acceptable salt thereof, wherein R^{1a} and R^{2a} are fluoro.

18. The compound of any one of claims 1 to 17, or a pharmaceutically acceptable salt thereof, wherein R^{1b} and R^{2b} are each independently selected from hydrogen, (C₁-C₄)alkyl, —[(C₁-C₄)alkyl]-OC(O)—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-OC(O)NR^{2c}R^{2d}], [(C₁-C₄)alkyl]-OC(O)O-[5- to 7-membered heterocyclyl], and phenyl, wherein the 5- to 7-membered heterocyclyl of [(C₁-C₄)alkyl]-OC(O)O-[5- to 7-membered heterocyclyl] is optionally substituted with C(O)OR^{2c}, wherein any of the (C₁-C₄)alkyl groups are optionally substituted with 1 or 2 (C₁-C₄)alkyl.

19. The compound of any one of claims 1 to 18, or a pharmaceutically acceptable salt thereof, wherein R^{1b} and R^{2b} are each —[(C₁-C₄)alkyl]-OC(O)—[(C₁-C₄)alkyl].

20. The compound of any one of claims 1 to 18, or a pharmaceutically acceptable salt thereof, wherein R^{1b} and R^{2b} are hydrogen.

21. The compound of any one of claims 1 to 15 and 18, or a pharmaceutically acceptable salt thereof, wherein —CR^{1a}R^{2a}P(O)OR^{1b}OR^{2b} is selected from 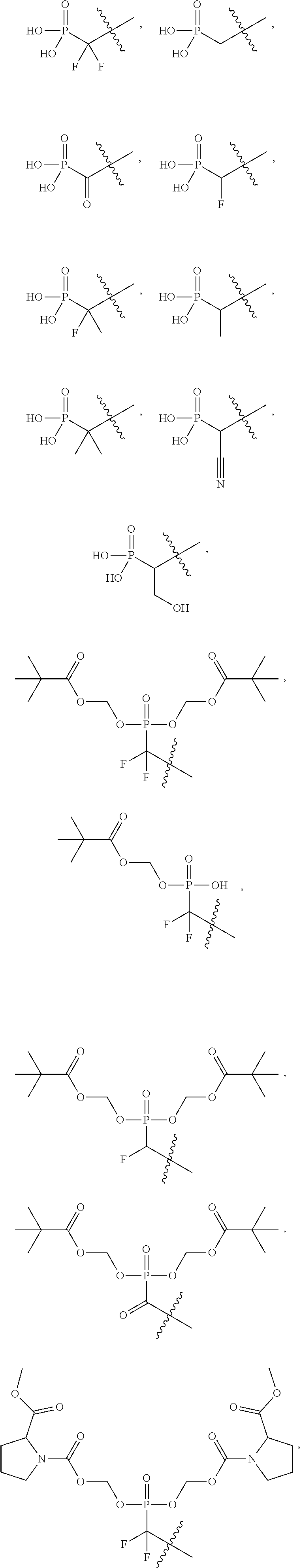 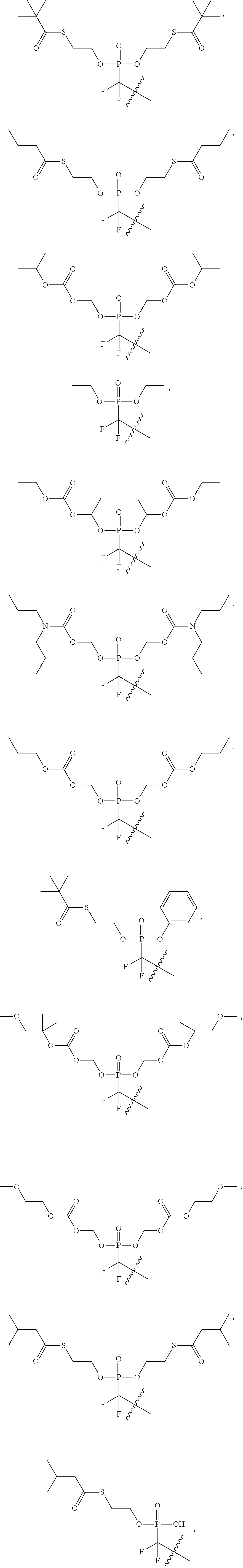 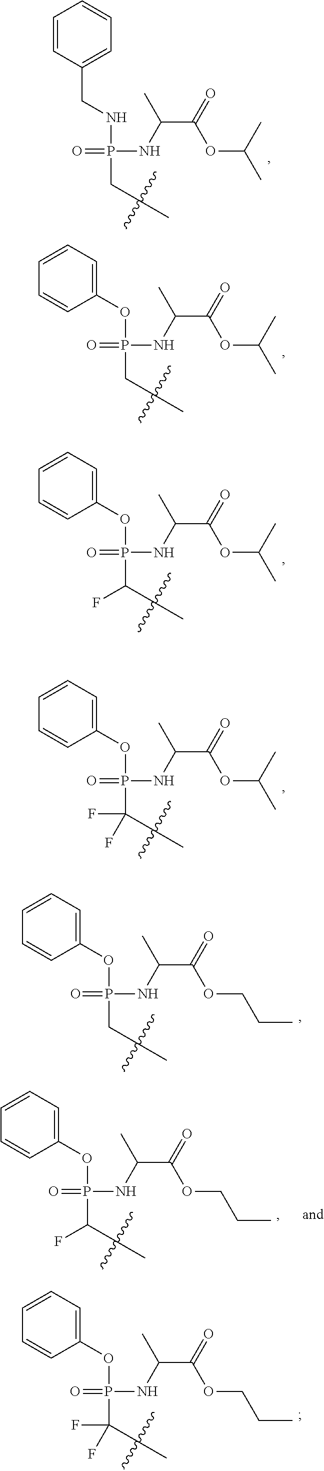
or a pharmaceutically acceptable salt thereof.

22. The compound of any one of claims 1 to 15, 18, and 21, or a pharmaceutically acceptable salt thereof, wherein —CR^{1a}R^{2a}P(O)OR^{1b}OR^{2b} is

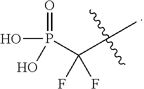 [SMILES: *C(F)(F)P(=O)(O)O]

23. The compound of any one of claims 1 to 22, or a pharmaceutically acceptable salt thereof, wherein R⁷ is selected from (C₁-C₄)alkyl, phenyl, 4- to 6-membered monocyclic heterocyclyl, 9- or 10-membered fused bicyclic heterocyclyl, 5- or 6-membered monocyclic heteroaryl, and 9- or 10-membered fused bicyclic heteroaryl, wherein said (C₁-C₄)alkyl is optionally substituted with, as valency permits, 1 to 3 groups selected from R^{Y} and said phenyl, 5- or 6-membered monocyclic heterocyclyl, 9- or 10-membered fused bicyclic heterocyclyl, 5- or 6-membered monocyclic heteroaryl, and 9- or 10-membered fused bicyclic heteroaryl are each optionally substituted with, as valency permits, 1 to 3 groups selected from R^{Z}.

24. The compound of any one of claims 1 to 23, or a pharmaceutically acceptable salt thereof, wherein R⁷ is selected from (C₁-C₄)alkyl, phenyl, pyridinyl, pyrimidinyl, naphthyl, cinnolinyl, quinoxalinyl, quinazolinyl, quinolinyl, isoquinolinyl, chromanyl, pyrazoyl, indazolyl, benzoisoxazolyl, imidazo[1,2-a]pyridinyl, pyrrolidinyl, cyclopentyl, cyclohexyl, azetidinyl, piperidinyl, and dihydropyridinyl, and wherein said (C₁-C₄)alkyl is optionally substituted with, as valency permits, 1 to 3 groups selected from R^{Y} and said phenyl, pyridinyl, pyrimidinyl, naphthyl, cinnolinyl, quinoxalinyl, quinazolinyl, quinolinyl, isoquinolinyl, chromanyl, pyrazoyl, indazolyl, benzoisoxazolyl, imidazo[1,2-a]pyridinyl, pyrrolidinyl, cyclopentyl, cyclohexyl, azetidinyl, piperidinyl, and dihydropyridinyl, are each optionally substituted with, as valency permits, 1 to 3 groups selected from R^{Z}.

25. The compound of any one of claims 1 to 24, or a pharmaceutically acceptable salt thereof, wherein R^{Y} is selected from cyano, (C₁-C₄)alkyl, —NR^{a}R^{b}, and hydroxyl.

26. The compound of any one of claims 1 to 25, or a pharmaceutically acceptable salt thereof, wherein R^{Z} is selected from halo, cyano, (C₁-C₄)alkyl, halo(C₁-C₄)alkyl, hydroxy(C₁-C₄)alkyl, —(C₁-C₄)alkylphenyl, —(C₁-C₄)alkylheteroaryl, (C₁-C₄)alkoxy, halo(C₁-C₄)alkoxy, —OR^{e}, oxo, phenyl, 4- to 6-membered heterocyclyl, —S(O)₂R^{f}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, and wherein said phenyl and said 4- to 6-membered heterocyclyl are each optionally and independently substituted with, as valency permits, 1 to 3 groups selected from cyano and oxo.

27. The compound of any one of claims 1 to 26, or a pharmaceutically acceptable salt thereof, wherein R^{Z} is selected halo, cyano, (C₁-C₄)alkyl, halo(C₁-C₄)alkyl, hydroxy(C₁-C₄)alkyl, —(C₁-C₄)alkylphenyl, —(C₁-C₄)alkylheteroaryl, (C₁-C₄)alkoxy, halo(C₁-C₄)alkoxy, —OR^{e}, oxo, —S(O)₂Rf, —(C₁-C₄alkyl)C(O)R^{g}, —C(O)R^{g}, —(C₁-C₄alkyl)C(O)NR^{c}R^{d}, —C(O)NR^{c}R^{d}, —NO₂, —NR^{a}R^{b}, phenyl, and dihydropyridinyl, wherein the (C₁-C₄)alkyl is optionally substituted with cyano, and wherein said phenyl and said dihydropyridinyl are each optionally and independently substituted with, as valency permits, 1 to 3 groups selected from cyano and oxo.

28. The compound of any one of claims 1 to 22, or a pharmaceutically acceptable salt thereof, wherein R⁶ and R⁷ together with the nitrogen atom to which they are attached form 4- to 6-membered monocyclic heterocyclyl, 7- to 13-membered spiro bicyclic heterocyclyl, or 9- to 10-membered fused bicyclic heterocyclyl, each of which being optionally substituted with, as valency permits, 1 to 3 groups selected from R^{Q}.

29. The compound of any one of claims 1 to 22 and 28, or a pharmaceutically acceptable salt thereof, wherein R⁶ and R⁷ together with the nitrogen atom to which they are attached form azetidinyl, spiro[indoline-3,3′-pyrrolidinyl], pyrrolidinyl, 3,4-dihydrobenzooxazinyl, 1,2,3,4-tetrahydroquinolinyl, octahydrothiopyranopyrroyl, octahydro-1H-thieno[3′,4′:3,4]cyclobuta[1,2-c]pyrroyl, 2,6-diazabicyclo[3.2.0]heptanyl, piperazinyl, morpholinyl, 1,4,5,6-tetrahydropyrrolopyrazoyl, 4-azaspiro[2.4]heptanyl, 2-oxa-5-azabicyclo[2.2.1]heptanyl, 7-oxa-4-azaspiro[2.5]octanyl, 8-oxa-3-azabicyclo[3.2.1]octanyl, 4,6-diazaspiro[2.4]heptanyl, 5-oxa-2,6-diazaspiro[3.4]oct-6-enyl, or piperidinyl, each of which being optionally substituted with, as valency permits, 1 to 3 groups selected from R^{Q}.

30. The compound of any one of claims 1 to 22 and 28, or a pharmaceutically acceptable salt thereof, wherein R⁶ and R⁷ together with the nitrogen atom to which they are attached form azetidinyl, spiro[indoline-3,3′-pyrrolidinyl], pyrrolidinyl, 3,4-dihydrobenzooxazinyl, 1,2,3,4-tetrahydroquinolinyl, octahydrothiopyranopyrroyl, octahydro-1H-thieno[3′,4′:3,4]cyclobuta[1,2-c]pyrroyl, 2,6-diazabicyclo[3.2.0]heptanyl, piperazinyl, 1,4,5,6-tetrahydropyrrolopyrazoyl, 5-oxa-2,6-diazaspiro[3.4]oct-6-enyl, or piperidinyl, each of which being optionally substituted with, as valency permits, 1 to 3 groups selected from R^{Q}.

31. The compound of any one of claims 1 to 22, 28, 29 and 30, or a pharmaceutically acceptable salt thereof, wherein R⁶ and R⁷ together with the nitrogen atom to which they are attached form pyrrolidinyl or azetidinyl, each of which being optionally substituted with, as valency permits, 1 to 3 groups selected from R^{Q}.

32. The compound of any one of claims 1 to 31, or a pharmaceutically acceptable salt thereof, wherein R^{Q} is selected from halo, (C₁-C₄)alkyl, halo(C₁-C₄)alkyl, —S(O)₂R^{f}, cyano, phenyl, 4- to 9-membered monocyclic or bicyclic heterocyclyl, 5- to 10-membered monocyclic or bicyclic heteroaryl, (C₃-C₆)cycloalkyl, oxo, —OR^{e}, —C(O)R^{g}, —C(O)NR^{c}R^{d}, —C(O)OR^{e}, —NR^{c}C(O)R^{e}, wherein said (C₁-C₄)alkyl are each optionally and independently substituted with, as valency permits, 1 to 3 groups selected from R^{M}, and wherein said phenyl, 4- to 6-membered heterocyclyl, 5- to 10-membered monocyclic or bicyclic heteroaryl, and (C₃-C₆)cycloalkyl are each optionally and independently substituted with, as valency permits, 1 to 3 groups selected from R^{F}.

33. The compound of any one of claims 1 to 31, or a pharmaceutically acceptable salt thereof, wherein R^{Q} is selected from halo, (C₁-C₄)alkyl, halo(C₁-C₄)alkyl, cyano, phenyl, 4- to 9-membered monocyclic or bicyclic heterocyclyl, 5- to 10-membered monocyclic or bicyclic heteroaryl, (C₃-C₆)cycloalkyl, oxo, —OR^{e}, —C(O)R^{g}, —C(O)NR^{c}R^{d}, —C(O)OR^{e}, —NR^{c}C(O)R^{e}, wherein said (C₁-C₄)alkyl are each optionally and independently substituted with, as valency permits, 1 to 3 groups selected from R^{M}, and wherein said phenyl, 4- to 6-membered heterocyclyl, 5- to 10-membered monocyclic or bicyclic heteroaryl, and (C₃-C₆)cycloalkyl are each optionally and independently substituted with, as valency permits, 1 to 3 groups selected from R^{F}.

34. The compound of any one of claims 1 to 33, or a pharmaceutically acceptable salt thereof, wherein R^{Q} is selected from cyano, (C₁-C₄)alkyl, halo(C₁-C₄)alkyl, —S(O)₂R^{f}, oxo, —C(O)R^{g}, —OR^{e}, —C(O)OR^{e}, —C(O)NR^{c}R^{d}, —NR^{c}C(O)R^{e}, cyclobutyl, cyclopropyl, cyclohexyl, phenyl, benzoisothiazoyl, pyridinyl, pyrimidinyl, dihydropyridinyl, pyrazoyl, piperidinyl, tetrahydroimidazopyridinyl, and isothiazolidinyl, wherein said (C₁-C₄)alkyl is optionally and independently substituted with, as valency permits, 1 to 3 groups selected from R^{M}, and wherein said cyclobutyl, cyclopropyl, cyclohexyl, phenyl, benzoisothiazoyl, pyridinyl, pyrimidinyl, dihydropyridinyl, pyrazoyl, piperidinyl, tetrahydroimidazopyridinyl, and isothiazolidinyl are each optionally and independently substituted with, as valency permits, 1 to 3 groups selected from R^{F}.

35. The compound of any one of claims 1 to 33, or a pharmaceutically acceptable salt thereof, wherein R^{Q} is selected from cyano, (C₁-C₄)alkyl, halo(C₁-C₄)alkyl, oxo, —C(O)R^{g}, —OR^{e}, —C(O)OR^{e}, —C(O)NR^{c}R^{d}, —NR^{c}C(O)R^{e}, cyclobutyl, cyclopropyl, cyclohexyl, phenyl, pyridinyl, pyrimidinyl, dihydropyridinyl, pyrazoyl, piperidinyl, tetrahydroimidazopyridinyl, and isothiazolidinyl, wherein said (C₁-C₄)alkyl is optionally and independently substituted with, as valency permits, 1 to 3 groups selected from R^{M}, and wherein said cyclobutyl, cyclopropyl, cyclohexyl, phenyl, pyridinyl, pyrimidinyl, dihydropyridinyl, pyrazoyl, piperidinyl, tetrahydroimidazopyridinyl, and isothiazolidinyl are each optionally and independently substituted with, as valency permits, 1 to 3 groups selected from R^{F}.

36. The compound of any one of claims 1 to 35, or a pharmaceutically acceptable salt thereof, wherein R^{M} is selected from phenyl and 5- to 6-membered monocyclic heteroaryl wherein said phenyl and 5- to 6-membered monocyclic heteroaryl, and phenyl are each optionally substituted with, as valency permits, 1 to 3 groups selected from R^{X}.

37. The compound of any one of claims 1 to 36, or a pharmaceutically acceptable salt thereof, wherein R^{M} is selected from phenyl and pyridinyl, wherein said phenyl and pyridinyl are each optionally substituted with, as valency permits, 1 to 3 groups selected from R^{X}.

38. The compound of any one of claims 1 to 37, or a pharmaceutically acceptable salt thereof, wherein R^{X} is selected from halo and (C₁-C₄)alkoxy.

39. The compound of any one of claims 1 to 38, or a pharmaceutically acceptable salt thereof, wherein R^{F} is selected from halo, cyano, (C₁-C₄)alkoxy, oxo, and —NR^{c}C(O)R^{e}.

40. The compound of any one of claims 1 to 39, or a pharmaceutically acceptable salt thereof, wherein R^{c} and R^{d} are each independently selected from, as valency permits, hydrogen and (C₁-C₄)alkyl, and wherein said (C₁-C₄)alkyl is optionally substituted with, as valency permits, 1 to 3 groups selected from R^{J}.

41. The compound of any one of claims 1 to 40, or a pharmaceutically acceptable salt thereof, wherein R^{e} is independently selected from hydrogen, phenyl, 5- to 6-membered heteroaryl, and (C₁-C₄)alkyl, and wherein said (C₁-C₄)alkyl is optionally substituted with, as valency permits, 1 to 3 groups selected from R^{J}.

42. The compound of any one of claims 1 to 41, or a pharmaceutically acceptable salt thereof, wherein R^{J} is independently selected from phenyl and 5- to 6-membered heteroaryl, and wherein the 5- to 6-membered heteroaryl is optionally substituted with a (C₃-C₆)cycloalkyl.

43. The compound of any one of claims 1 to 42, or a pharmaceutically acceptable salt thereof, wherein R^{J} is phenyl.

44. The compound of any one of claims 1 to 43, or a pharmaceutically acceptable salt thereof, wherein R⁹ is selected from piperidinyl, morpholinyl, and piperizinyl.

45. The compound of any one of claims 1 to 9 or 11 to 44, or a pharmaceutically acceptable salt thereof, wherein X is NR⁸.

46. The compound of any one of claims 1 to 45, wherein:
R⁸ is hydrogen, —C(O)R^{Ha}, —C(O)OR^{Ha}, (C₁-C₄)alkyl, —SO₂R^{Ha}, —C(O)NR^{Ha}R^{Hb}, halo(C₁-C₄)alkyl, phenyl, —(C₁-C₄)[4- to 10-membered monocyclic or bicyclic heterocyclyl], —(C₁-C₄)[5- to 10-membered monocyclic or bicyclicheteroaryl], —(C₃-C₆)cycloalkyl, or 4- to 10-membered monocyclic or bicyclic heterocyclyl, wherein the phenyl on the —(C₁-C₄)alkylphenyl is optionally substituted with 1 or 2 groups independently selected from —(C₁₋₃alkoxy) and 5- to 6-membered heteroaryl optionally substituted with —(C₁-C₄)alkyl, and wherein the (C₁-C₄)alkyl is optionally substituted with 1 to 3 phenyl;
R^{Ha} is (C₁-C₄)alkyl, —(C₁-C₄)[5- to 6-membered heteroaryl], —(C₁-C₄)[phenyl], —(C₁-C₄)[(C₃-C₆)cycloalkyl], phenyl, 4- to 10-membered monocyclic or bicyclic heterocyclyl, wherein the phenyl and 4- to 10-membered monocyclic or bicyclic heterocyclyl are each optionally substituted with 1 to 3 groups independently selected from halo, (C₁-C₄)alkyl, (C₁-C₄)alkoxy, and 4- to 6-membered monocyclic heterocyclyl optionally substituted with C₁-C₄alkyl; and
R^{Hb} is H.

47. The compound of claim 46, wherein:
R⁸ is —H, —CH₂CH₃, —CH₂CH(CH₃)₂, —CH₂CF₂, —CH₂CF₃, cyclobutyl, —CH₂— quinuclidinyl, —CH₂-imidazo[1,5-a]pyridinyl, —CH₂-pyrimidinyl, —CH₂-phenyl, phenyl, pyrimidinyl, pyridinyl, isoquinolinyl, —C(O)R^{Ha}, —C(O)OR^{Ha}, —SO₂R^{Ha}, or —C(O)NR^{Ha}R^{Hb}, or R⁸ is represented by the following structure: 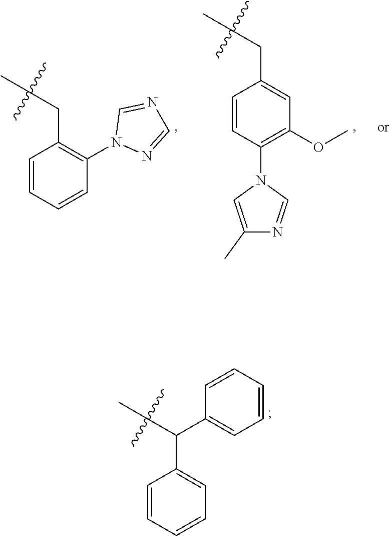
and
R^{Ha} is methyl, ethyl, propyl, isopropyl, —CH₂-quinolinyl, —CH₂-phenyl, —CH₂CH₂-phenyl, —CH(CH₃)-phenyl, —CH(CH₃)CH₂-phenyl, —CH₂CH(CH₃)-phenyl, —CH₂CH₂-pyrimidinyl, —CH₂C(CH₃)₂-cyclohexyl, quinolinyl, indazoyl, benzoisoxazolyl, imidazo[1,5-a]pyridinyl, pyrazolo[1,5-a]pyrimidinyl, 5,6,7,8-tetrahydroimidazo[1,5-a]pyridinyl, 4,5,6,7-tetrahydrobenzo[d]isoxazolyl, or 6,7-dihydro-4H-pyrano[3,4-d]isoxazolyl, and wherein the quinolinyl, indazoyl, benzoisoxazolyl, imidazo[1,5-a]pyridinyl, pyrazolo[1,5-a]pyrimidinyl, 5,6,7,8-tetrahydroimidazo[1,5-a]pyridinyl, 4,5,6,7-tetrahydrobenzo[d]isoxazolyl, 6,7-dihydro-4H-pyrano[3,4-d]isoxazolyl are each optionally substituted with 1 to 3 groups independently selected from —Cl, —CH₃, —CH₂CH₃, —OCH₃, or N-methylpiperazinyl,

48. The compound of claim 1, wherein the compound is selected any one of Compounds 1 to 441; or a pharmaceutically acceptable salt thereof.

49. A pharmaceutically acceptable composition comprising the compound of any one of claims 1 to 48, or a pharmaceutically acceptable salt thereof; and a pharmaceutically acceptable carrier.

50. A method of treating a condition responsive to the modulation of STAT3 or STAT6 in a subject comprising administering to the subject a therapeutically effective amount of the compound of any one of claims 1 to 48 or a pharmaceutically acceptable salt thereof, or the pharmaceutically acceptable composition of claim 49.
